(19) United States
(12) Patent Application Publication
(10) Pub. No.: US 2023/0016656 A1
(43) Pub. Date: Jan. 19, 2023

(54) NOVEL CRISPR DNA TARGETING ENZYMES AND SYSTEMS

(71) Applicant: ARBOR BIOTECHNOLOGIES, INC., Cambridge, MA (US)

(72) Inventors: David A. Scott, Cambridge, MA (US); David R. Cheng, Boston, MA (US); Winston X. Yan, Boston, MA (US); Tia M. Ditommaso, Waltham, MA (US)

(21) Appl. No.: 17/638,065

(22) PCT Filed: Aug. 26, 2020
(86) PCT No.: PCT/US2020/048037

§ 371 Date: Feb. 24, 2022

Related U.S. Application Data
(60) Provisional application No. 62/896,277, filed on Sep. 5, 2019.
(60) Provisional application No. 62/893,064, filed on Aug. 28, 2019.
(60) Provisional application No. 62/893,059, filed on Aug. 28, 2019.
(60) Provisional application No. 62/892,358, filed on Aug. 27, 2019.
(60) Provisional application No. 62/892,382, filed on Aug. 27, 2019.
(60) Provisional application No. 62/892,390, filed on Aug. 27, 2019.
(60) Provisional application No. 62/892,446, filed on Aug. 27, 2019.
(60) Provisional application No. 62/892,434, filed on Aug. 27, 2019.

(51) Int. Cl. C12N 9/22; C12N 15/11; C12N 15/90

(52) U.S. Cl. CPC C12N 9/22; C12N 15/11; C12N 15/907; C12N 2310/20

(57) ABSTRACT

The disclosure describes novel systems, methods, and compositions for the manipulation of nucleic acids in a targeted fashion. The disclosure describes non-naturally occurring, engineered CRISPR systems, components, and methods for targeted modification of nucleic acids. Each system includes one or more protein components and one or more nucleic acid components that together target nucleic acids.

### RELATED APPLICATIONS

This application claims priority to U.S. Provisional Application 62/892,358 filed on Aug. 27, 2019, U.S. Provisional Application 62/892,382 filed on Aug. 27, 2019, U.S. Provisional Application 62/892,390 filed on Aug. 27, 2019, U.S. Provisional Application filed on 62/892,446 filed on Aug. 27, 2019, U.S. Provisional Application 62/892,434 filed on Aug. 27, 2019, U.S. Provisional Application 62/893,064 filed on Aug. 28, 2019, U.S. Provisional Application 62/893,059 filed on Aug. 28, 2019, and U.S. Provisional Application 62/896,277 filed on Sep. 5, 2019, the entire contents of each of which are hereby incorporated by reference.

### SEQUENCE LISTING

The instant application contains a Sequence Listing which has been submitted electronically in ASCII format and is hereby incorporated by reference in its entirety. Said ASCII copy, created on Aug. 26, 2020 is named A2186-7018WO_SL.txt and is 1,301,348 bytes in size.

### FIELD OF THE INVENTION

The present disclosure relates to systems and methods for genome editing and modulation of gene expression using novel Clustered Regularly Interspaced Short Palindromic Repeats (CRISPR) and CRISPR-associated (Cas) genes.

### BACKGROUND

Recent advances in genome sequencing technologies and analyses have yielded significant insight into the genetic underpinnings of biological activities in many diverse areas of nature, ranging from prokaryotic biosynthetic pathways to human pathologies. To fully understand and evaluate the vast quantities of information yielded, equivalent increases in the scale, efficacy, and ease of sequence technologies for genome and epigenome manipulation are needed. These novel technologies will accelerate the development of novel applications in numerous areas, including biotechnology, agriculture, and human therapeutics.

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements. CRISPR-Cas systems comprise an extremely diverse group of proteins effectors, non-coding elements, and loci architectures, some examples of which have been engineered and adapted to produce important biotechnological advances.

The components of the system involved in host defense include one or more effector proteins capable of modifying a nucleic acid and an RNA guide element that is responsible for targeting the effector protein(s) to a specific sequence on a phage nucleic acid. The RNA guide is composed of a CRISPR RNA (crRNA) and may require an additional trans-activating RNA (tracrRNA) to enable targeted nucleic acid manipulation by the effector protein(s). The crRNA consists of a direct repeat responsible for protein binding to the crRNA and a spacer sequence that is complementary to the desired nucleic acid target sequence. CRISPR systems can be reprogrammed to target alternative DNA or RNA targets by modifying the spacer sequence of the crRNA.

CRISPR-Cas systems can be broadly classified into two classes: Class 1 systems are composed of multiple effector proteins that together form a complex around a crRNA, and Class 2 systems consist of one effector protein that complexes with the RNA guide to target nucleic acid substrates. The single-subunit effector composition of the Class 2 systems provides a simpler component set for engineering and application translation and have thus far been an important source of programmable effectors. Nevertheless, there remains a need for additional programmable effectors and systems for modifying nucleic acids and polynucleotides (i.e., DNA, RNA, or any hybrid, derivative, or modification) beyond the current CRISPR-Cas systems, such as smaller effectors and/or effectors having unique PAM sequence requirements, that enable novel applications through their unique properties.

### SUMMARY

This disclosure provides non-naturally-occurring, engineered systems and compositions for novel single-effector Class 2 CRISPR-Cas systems, which were first identified computationally from genomic databases and subsequently engineered and experimentally validated. In particular, identification of the components of these CRISPR-Cas systems allows for their use in non-natural environments, e.g., in bacteria other than those in which the systems were initially discovered or in eukaryotic cells, such as mammalian cells. These new effectors are divergent in sequence and function compared to orthologs and homologs of existing Class 2 CRISPR effectors.

In one aspect, the disclosure provides engineered, non-naturally occurring Clustered Regularly Interspaced Short Palindromic Repeat (CRISPR)—Cas systems of CLUST.133120 including: a CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 1-50; and an RNA guide including a direct repeat sequence and a spacer sequence capable of hybridizing to a target nucleic acid; wherein the CRISPR-associated protein is capable of binding to the RNA guide and of modifying the target nucleic acid sequence complementary to the spacer sequence. In one aspect, the disclosure provides engineered, non-naturally occurring Clustered Regularly Interspaced Short Palindromic Repeat (CRISPR)—Cas systems of CLUST.133120 including: a CRISPR-associated protein or a nucleic acid encoding the CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 1-50; and an RNA guide including a direct repeat sequence and a spacer sequence capable of hybridizing to a target nucleic acid, or a nucleic acid encoding the RNA guide; wherein the CRISPR-associated protein is capable of binding to the RNA guide and of modifying the target nucleic acid sequence complementary to the spacer sequence.

In some embodiments of any of the systems described herein, the direct repeat sequence includes a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 51-72, 85-87, 95-100, or 900-915.

In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 1, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 51, 95, or 85. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence of Table 3 and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a corresponding direct repeat nucleotide sequence listed in Table 4 (e.g., the first or second direct repeat nucleotide sequence of the corresponding row in Table 4), or to a corresponding direct repeat nucleotide sequence listed in Table 32 (e.g., a pre-crRNA direct repeat sequence or a mature crRNA direct repeat sequence of Table 32).

In some embodiments of any of the systems described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 1 (CLUST.133120 3300027740) or SEQ ID NO: 2 (CLUST.133120 3300017971).

In some embodiments of any of the systems described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 1-50 (CLUST.133120).

In some embodiments of any of the systems described herein, the CRISPR-associated protein is capable of recognizing a protospacer adjacent motif (PAM), wherein the PAM includes a nucleic acid sequence, including a nucleic acid sequence set forth as 5′-TTN-3′ or 5′-TN-3′.

In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO:1, and the PAM sequence comprises a nucleic acid sequence set forth as 5′-TTN-3′. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 2, and the PAM sequence comprises a nucleic acid sequence set forth as 5′-TN-3′.

In some embodiments of any of the systems described herein, the spacer sequence of the RNA guide includes between about 15 nucleotides to about 55 nucleotides. In some embodiments of any of the systems described herein, the spacer sequence of the RNA guide includes between 20 and 35 nucleotides.

In another aspect, the disclosure provides a cell (e.g., a genetically modified cell), wherein the cell includes: a CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 1-50; and an RNA guide including a direct repeat sequence and a spacer sequence capable of hybridizing to a target nucleic acid. In another aspect, the disclosure provides a cell (e.g., a genetically modified cell), wherein the cell includes: a CRISPR-associated protein or a nucleic acid encoding the CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 1-50; and an RNA guide including a direct repeat sequence and a spacer sequence capable of hybridizing to a target nucleic acid, or a nucleic acid encoding the RNA guide.

In some embodiments of any of the cells described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 1 or SEQ ID NO: 2.

In some embodiments of any of the cells described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 1-50.

In some embodiments of any of the cells described herein, the CRISPR-associated protein is capable of recognizing a PAM sequence including a nucleic acid sequence set forth as 5′-TTN-3′ or 5′-TN-3′.

In some embodiments of any of the cells described herein, the direct repeat sequence includes a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 51-72, 85-87, 95-100, or 900-915.

In some embodiments of any of the cells described herein, the spacer sequence includes between about 15 nucleotides to about 55 nucleotides. In some embodiments of any of the cells described herein, the spacer sequence includes between 20 and 35 nucleotides.

In another aspect, the disclosure provides methods of modifying a target nucleic acid, the method including delivering to the target nucleic acid an engineered, non-naturally occurring CRISPR-Cas system including: a CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 1-50; and an RNA guide including a direct repeat sequence and a spacer sequence capable of hybridizing to the target nucleic acid; wherein the CRISPR-associated protein is capable of binding to the RNA guide; and wherein recognition of the target nucleic acid by the CRISPR-associated protein and RNA guide results in a modification of the target nucleic acid. In another aspect, the disclosure provides methods of modifying a target nucleic acid, the method including delivering to the target nucleic acid an engineered, non-naturally occurring CRISPR-Cas system including: a CRISPR-associated protein or a nucleic acid encoding the CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 1-50; and an RNA guide including a direct repeat sequence and a spacer sequence capable of hybridizing to the target nucleic acid, or a nucleic acid encoding the RNA guide; wherein the CRISPR-associated protein is capable of binding to the RNA guide; and wherein recognition of the target nucleic acid by the CRISPR-associated protein and RNA guide results in a modification of the target nucleic acid.

In some embodiments of any of the methods described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 1 or SEQ ID NO: 2.

In some embodiments of any of the methods described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 1-50 (CLUST.133120).

In some embodiments of any of the methods described herein, the CRISPR-associated protein is capable of recognizing a PAM sequence including a nucleic acid sequence set forth as 5′-TTN-3′ or 5′-TN-3′.

In some embodiments of any of the methods described herein, the direct repeat sequence includes a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 51-72, 85-87, 95-100, or 900-915.

In some embodiments of any of the methods described herein, the spacer sequence includes between about 15 nucleotides to about 55 nucleotides. In some embodiments of any of the methods described herein, the spacer sequence includes between 20 and 35 nucleotides.

In one aspect, the disclosure provides engineered, non-naturally occurring Clustered Regularly Interspaced Short Palindromic Repeat (CRISPR)—Cas systems of CLUST.099129 including: a CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 101-145; and an RNA guide including a direct repeat sequence and a spacer sequence capable of hybridizing to a target nucleic acid; wherein the CRISPR-associated protein is capable of binding to the RNA guide and of modifying the target nucleic acid sequence complementary to the spacer sequence. In one aspect, the disclosure provides engineered, non-naturally occurring Clustered Regularly Interspaced Short Palindromic Repeat (CRISPR)—Cas systems of CLUST.099129 including: a CRISPR-associated protein or a nucleic acid encoding the CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 101-145; and an RNA guide including a direct repeat sequence and a spacer sequence capable of hybridizing to a target nucleic acid, or a nucleic acid encoding the RNA guide; wherein the CRISPR-associated protein is capable of binding to the RNA guide and of modifying the target nucleic acid sequence complementary to the spacer sequence.

In some embodiments of any of the systems described herein, the direct repeat sequence includes a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 146-162, 180-183, or 200-215.

In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 101, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 146, 181, or 200.

In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence of Table 10, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a corresponding direct repeat nucleotide sequence listed in Table 11 (e.g., the first or second direct repeat nucleotide sequence of the corresponding row in Table 11), or to a corresponding direct repeat nucleotide sequence listed in Table 7 (e.g., a pre-crRNA Direct Repeat Sequence or a Mature crRNA Direct Repeat Sequence of Table 7).

In some embodiments of any of the systems described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 101 (CLUST.099129 SRR6837557), SEQ ID NO: 102 (CLUST.099129 3300012971), or SEQ ID NO: 103 (CLUST.099129 3300005764).

In some embodiments of any of the systems described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 101-145 (CLUST.0991297).

In some embodiments of any of the systems described herein, the CRISPR-associated protein is capable of recognizing a protospacer adjacent motif (PAM), wherein the PAM includes a nucleic acid sequence, including a nucleic acid sequence set forth as 5′-GTN-3′, 5′-TG-3′, 5′-TR-3′, or 5′-RATG-3′ (SEQ ID NO: 920).

In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 101, and the PAM sequence comprises a nucleic acid sequence set forth as 5′-GTN-3′. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 102, and the PAM sequence comprises a nucleic acid sequence set forth as 5′-TG-3′. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 103, and the PAM sequence comprises a nucleic acid sequence set forth as 5′-TR-3′ or 5′-RATG-3′ (SEQ ID NO: 920).

In some embodiments of any of the systems described herein, the spacer sequence of the RNA guide includes between about 15 nucleotides to about 55 nucleotides. In some embodiments of any of the systems described herein, the spacer sequence of the RNA guide includes between 26 and 51 nucleotides.

In another aspect, the disclosure provides a cell (e.g., a genetically modified cell), wherein the cell includes: a CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 101-145; and RNA guide including a direct repeat sequence and a spacer sequence capable of hybridizing to a target nucleic acid. In another aspect, the disclosure provides a cell (e.g., a genetically modified cell), wherein the cell includes: a CRISPR-associated protein or a nucleic acid encoding the CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 101-145; and an RNA guide including a direct repeat sequence and a spacer sequence capable of hybridizing to a target nucleic acid, or a nucleic acid encoding the RNA guide.

In some embodiments of any of the cells described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 101, SEQ ID NO: 102, or SEQ ID NO: 103.

In some embodiments of any of the cells described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 101-145.

In some embodiments of any of the cells described herein, the CRISPR-associated protein is capable of recognizing a PAM sequence including a nucleic acid sequence set forth as 5′-GTN-3′, 5′-TG-3′, 5′-TR-3′, or 5′-RATG-3′ (SEQ ID NO: 920).

In some embodiments of any of the cells described herein, the direct repeat sequence includes a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 146-162, 180-183, or 200-215.

In some embodiments of any of the cells described herein, the spacer sequence includes between about 15 nucleotides to about 55 nucleotides. In some embodiments of any of the cells described herein, the spacer sequence includes between 26 and 51 nucleotides.

In another aspect, the disclosure provides methods of modifying a target nucleic acid, the method including delivering to the target nucleic acid an engineered, non-naturally occurring CRISPR-Cas system including: a CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 101-145; and an RNA guide including a direct repeat sequence and a spacer sequence capable of hybridizing to the target nucleic acid; wherein the CRISPR-associated protein is capable of binding to the RNA guide; and wherein recognition of the target nucleic acid by the CRISPR-associated protein and RNA guide results in a modification of the target nucleic acid. In another aspect, the disclosure provides methods of modifying a target nucleic acid, the method including delivering to the target nucleic acid an engineered, non-naturally occurring CRISPR-Cas system including: a CRISPR-associated protein or a nucleic acid encoding the CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 101-145; and an RNA guide including a direct repeat sequence and a spacer sequence capable of hybridizing to the target nucleic acid, or a nucleic acid encoding the RNA guide; wherein the CRISPR-associated protein is capable of binding to the RNA guide; and wherein recognition of the target nucleic acid by the CRISPR-associated protein and RNA guide results in a modification of the target nucleic acid.

In some embodiments of any of the methods described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 101, SEQ ID NO: 102, or SEQ ID NO: 103.

In some embodiments of any of the methods described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 101-145.

In some embodiments of any of the methods described herein, the CRISPR-associated protein is capable of recognizing a PAM sequence including a nucleic acid sequence set forth as 5′-GTN-3′, 5′-TG-3′, 5′-TR-3′, or 5′-RATG-3′.

In some embodiments of any of the methods described herein, the direct repeat sequence includes a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 146-162, 180-183, or 200-215.

In some embodiments of any of the methods described herein, the spacer sequence includes between about 15 nucleotides to about 55 nucleotides. In some embodiments of any of the methods described herein, the spacer sequence includes between 26 and 51 nucleotides.

In one aspect, the disclosure provides engineered, non-naturally occurring Clustered Regularly Interspaced Short Palindromic Repeat (CRISPR)—Cas systems of CLUST.342201 including: a CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 301-341; and an RNA guide including a direct repeat sequence and a spacer sequence capable of hybridizing to a target nucleic acid; wherein the CRISPR-associated protein is capable of binding to the RNA guide and of modifying the target nucleic acid sequence complementary to the spacer sequence. In one aspect, the disclosure provides engineered, non-naturally occurring Clustered Regularly Interspaced Short Palindromic Repeat (CRISPR)—Cas systems of CLUST.342201 including: a CRISPR-associated protein or a nucleic acid encoding the CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 301-341; and an RNA guide including a direct repeat sequence and a spacer sequence capable of hybridizing to a target nucleic acid, or a nucleic acid encoding the RNA guide; wherein the CRISPR-associated protein is capable of binding to the RNA guide and of modifying the target nucleic acid sequence complementary to the spacer sequence.

In some embodiments of any of the systems described herein, the direct repeat sequence includes a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 342-362, 384-402, 451, 452, or 454.

In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 301, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 342 or 451.

In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence of Table 17, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a corresponding direct repeat nucleotide sequence listed in Table 14, or to a corresponding direct repeat nucleotide sequence listed in Table 18 (e.g., the first or second direct repeat nucleotide sequence of the corresponding row in Table 18).

In some embodiments of any of the systems described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 301 (CLUST.342201 3300006417).

In some embodiments of any of the systems described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 301-341 (CLUST.342201).

In some embodiments of any of the systems described herein, the CRISPR-associated protein is capable of recognizing a protospacer adjacent motif (PAM), wherein the PAM includes a nucleic acid sequence, including a nucleic acid sequence set forth as 5′-AAG-3′, 5′-AAD-3′, 5′-AAR-3′, 5′-RAAG-3′ (SEQ ID NO: 921), 5′-RAAR-3′ (SEQ ID NO: 922), or 5′-RAAD-3′ (SEQ ID NO: 923).

In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO:301, and the PAM sequence comprises a nucleic acid sequence set forth as 5′-AAG-3′, 5′-AAD-3′, or 5′-AAR-3′.

In some embodiments of any of the systems described herein, the spacer sequence of the RNA guide includes between about 12 nucleotides to about 62 nucleotides. In some embodiments of any of the systems described herein, the spacer sequence of the RNA guide includes between 19 and 40 nucleotides.

In another aspect, the disclosure provides a cell (e.g., a genetically modified cell), wherein the cell includes: a CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 301-341; an RNA guide including a direct repeat sequence and a spacer sequence capable of hybridizing to a target nucleic acid. In another aspect, the disclosure provides a cell (e.g., a genetically modified cell), wherein the cell includes: a CRISPR-associated protein or a nucleic acid encoding the CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 301-341; and an RNA guide including a direct repeat sequence and a spacer sequence capable of hybridizing to a target nucleic acid, or a nucleic acid encoding the RNA guide.

In some embodiments of any of the cells described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 301.

In some embodiments of any of the cells described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 301-341.

In some embodiments of any of the cells described herein, the CRISPR-associated protein is capable of recognizing a PAM sequence including a nucleic acid sequence set forth as 5′-AAG-3′, 5′-AAD-3′, 5′-AAR-3′, 5′-RAAG-3′ (SEQ ID NO: 921), 5′-RAAR-3′ (SEQ ID NO: 922), 5′-RAAD-3′ (SEQ ID NO: 923).

In some embodiments of any of the cells described herein, the direct repeat sequence includes a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 342-362, 384-402, 451, 452, or 454.

In some embodiments of any of the cells described herein, the spacer sequence includes between about 12 nucleotides to about 62 nucleotides. In some embodiments of any of the cells described herein, the spacer sequence includes between 19 and 40 nucleotides.

In another aspect, the disclosure provides methods of modifying a target nucleic acid, the method including delivering to the target nucleic acid an engineered, non-naturally occurring CRISPR-Cas system including: a CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 301-341; and an RNA guide including a direct repeat sequence and a spacer sequence capable of hybridizing to the target nucleic acid; wherein the CRISPR-associated protein is capable of binding to the RNA guide; and wherein recognition of the target nucleic acid by the CRISPR-associated protein and RNA guide results in a modification of the target nucleic acid. In another aspect, the disclosure provides methods of modifying a target nucleic acid, the method including delivering to the target nucleic acid an engineered, non-naturally occurring CRISPR-Cas system including: a CRISPR-associated protein or a nucleic acid encoding the CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 301-341; and an RNA guide including a direct repeat sequence and a spacer sequence capable of hybridizing to the target nucleic acid, or a nucleic acid encoding the RNA guide; wherein the CRISPR-associated protein is capable of binding to the RNA guide; and wherein recognition of the target nucleic acid by the CRISPR-associated protein and RNA guide results in a modification of the target nucleic acid.

In some embodiments of any of the methods described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 301.

In some embodiments of any of the methods described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 301-341.

In some embodiments of any of the methods described herein, the CRISPR-associated protein is capable of recognizing a PAM sequence including a nucleic acid sequence set forth as 5′-AAG-3′, 5′-AAD-3′, 5′-AAR-3′, 5′-RAAG-3′ (SEQ ID NO: 921), 5′-RAAR-3′ (SEQ ID NO: 922), 5′-RAAD-3′ (SEQ ID NO: 923).

In some embodiments of any of the methods described herein, the direct repeat sequence includes a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 342-362, 384-402, 451, 452, or 454.

In some embodiments of any of the methods described herein, the spacer sequence includes between about 12 nucleotides to about 62 nucleotides. In some embodiments of any of the methods described herein, the spacer sequence includes between 19 and 40 nucleotides.

In one aspect, the disclosure provides engineered, non-naturally occurring Clustered Regularly Interspaced Short Palindromic Repeat (CRISPR)—Cas systems of CLUST.195009 including: a CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 501-521; and an RNA guide including a direct repeat sequence and a spacer sequence capable of hybridizing to a target nucleic acid; wherein the CRISPR-associated protein is capable of binding to the RNA guide and of modifying the target nucleic acid sequence complementary to the spacer sequence. In one aspect, the disclosure provides engineered, non-naturally occurring Clustered Regularly Interspaced Short Palindromic Repeat (CRISPR)—Cas systems of CLUST.195009 including: a CRISPR-associated protein or a nucleic acid encoding the CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 501-521; and an RNA guide including a direct repeat sequence and a spacer sequence capable of hybridizing to a target nucleic acid, or a nucleic acid encoding the RNA guide; wherein the CRISPR-associated protein is capable of binding to the RNA guide and of modifying the target nucleic acid sequence complementary to the spacer sequence.

In some embodiments of any of the systems described herein, the direct repeat sequence includes a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 522-532, 535, or 539-549.

In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 501, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 522 or 539.

In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence of Table 23, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a corresponding direct repeat nucleotide sequence listed in Table 20, or to a corresponding direct repeat nucleotide sequence listed in Table 24 (e.g., the first or second direct repeat nucleotide sequence of the corresponding row in Table 24).

In some embodiments of any of the systems described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 501 (CLUST.195009 SRR6201554).

In some embodiments of any of the systems described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 501-521 (CLUST.195009).

In some embodiments of any of the systems described herein, the CRISPR-associated protein is capable of recognizing a protospacer adjacent motif (PAM), wherein the PAM includes a nucleic acid sequence, including a nucleic acid sequence set forth as 5′-TTN-3′.

In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO:501, and the PAM sequence comprises a nucleic acid sequence set forth as 5′-TTN-3′.

In some embodiments of any of the systems described herein, the spacer sequence of the RNA guide includes between about 15 nucleotides to about 55 nucleotides. In some embodiments of any of the systems described herein, the spacer sequence of the RNA guide includes between 20 and 39 nucleotides.

In another aspect, the disclosure provides a cell (e.g., a genetically modified cell), wherein the cell includes: a CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 501-521; an RNA guide including a direct repeat sequence and a spacer sequence capable of hybridizing to a target nucleic acid. In another aspect, the disclosure provides a cell (e.g., a genetically modified cell), wherein the cell includes: a CRISPR-associated protein or a nucleic acid encoding the CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 501-521; and an RNA guide including a direct repeat sequence and a spacer sequence capable of hybridizing to a target nucleic acid, or a nucleic acid encoding the RNA guide.

In some embodiments of any of the cells described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 501.

In some embodiments of any of the cells described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 501-521.

In some embodiments of any of the cells described herein, the CRISPR-associated protein is capable of recognizing a PAM sequence including a nucleic acid sequence set forth as 5′-TTN-3′.

In some embodiments of any of the cells described herein, the direct repeat sequence includes a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 522-532, 535, or 539-549.

In some embodiments of any of the cells described herein, the spacer sequence includes between about 15 nucleotides to about 55 nucleotides. In some embodiments of any of the cells described herein, the spacer sequence includes between 20 and 39 nucleotides.

In another aspect, the disclosure provides methods of modifying a target nucleic acid, the method including delivering to the target nucleic acid an engineered, non-naturally occurring CRISPR-Cas system including: a CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 501-521; and an RNA guide including a direct repeat sequence and a spacer sequence capable of hybridizing to the target nucleic acid; wherein the CRISPR-associated protein is capable of binding to the RNA guide; and wherein recognition of the target nucleic acid by the CRISPR-associated protein and RNA guide results in a modification of the target nucleic acid. In another aspect, the disclosure provides methods of modifying a target nucleic acid, the method including delivering to the target nucleic acid an engineered, non-naturally occurring CRISPR-Cas system including: a CRISPR-associated protein or a nucleic acid encoding the CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 501-521; and an RNA guide including a direct repeat sequence and a spacer sequence capable of hybridizing to the target nucleic acid, or a nucleic acid encoding the RNA guide; wherein the CRISPR-associated protein is capable of binding to the RNA guide; and wherein recognition of the target nucleic acid by the CRISPR-associated protein and RNA guide results in a modification of the target nucleic acid.

In some embodiments of any of the methods described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 501.

In some embodiments of any of the methods described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 501-521.

In some embodiments of any of the methods described herein, the CRISPR-associated protein is capable of recognizing a PAM sequence including a nucleic acid sequence set forth as 5′-TTN-3′.

In some embodiments of any of the methods described herein, the direct repeat sequence includes a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 522-532, 535, or 539-549.

In some embodiments of any of the methods described herein, the spacer sequence includes between about 15 nucleotides to about 55 nucleotides. In some embodiments of any of the methods described herein, the spacer sequence includes between 20 and 39 nucleotides.

In one aspect, the disclosure provides engineered, non-naturally occurring Clustered Regularly Interspaced Short Palindromic Repeat (CRISPR)—Cas systems of CLUST.057059 including: a CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 601-682; and an RNA guide including a direct repeat sequence and a spacer sequence capable of hybridizing to a target nucleic acid; wherein the CRISPR-associated protein is capable of binding to the RNA guide and of modifying the target nucleic acid sequence complementary to the spacer sequence. In one aspect, the disclosure provides engineered, non-naturally occurring Clustered Regularly Interspaced Short Palindromic Repeat (CRISPR)—Cas systems of CLUST.057059 including: a CRISPR-associated protein or a nucleic acid encoding the CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 601-682; and an RNA guide including a direct repeat sequence and a spacer sequence capable of hybridizing to a target nucleic acid, or a nucleic acid encoding the RNA guide; wherein the CRISPR-associated protein is capable of binding to the RNA guide and of modifying the target nucleic acid sequence complementary to the spacer sequence.

In some embodiments of any of the systems described herein, the direct repeat sequence includes a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 683-734 or 751-802.

In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 601, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 683 or 751.

In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence of Table 29, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a corresponding direct repeat nucleotide sequence listed in Table 26, or to a corresponding direct repeat nucleotide sequence listed in Table 30 (e.g., the first or second direct repeat nucleotide sequence of the corresponding row in Table 30).

In some embodiments of any of the systems described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 601 (CLUST.057059 3300023179).

In some embodiments of any of the systems described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 601-682 (CLUST.057059).

In some embodiments of any of the systems described herein, the CRISPR-associated protein is capable of recognizing a protospacer adjacent motif (PAM), wherein the PAM includes a nucleic acid sequence, including a nucleic acid sequence set forth as 5′-GTN-3′.

In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 601, and the PAM sequence comprises a nucleic acid sequence set forth as 5′-GTN-3′.

In some embodiments of any of the systems described herein, the spacer sequence of the RNA guide includes between about 15 nucleotides to about 50 nucleotides. In some embodiments of any of the systems described herein, the spacer sequence of the RNA guide includes between 20 and 44 nucleotides.

In another aspect, the disclosure provides a cell (e.g., a genetically modified cell), wherein the cell includes: a CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 601-682; an RNA guide including a direct repeat sequence and a spacer sequence capable of hybridizing to a target nucleic acid. In another aspect, the disclosure provides a cell (e.g., a genetically modified cell), wherein the cell includes: a CRISPR-associated protein or a nucleic acid encoding the CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 601-682; and an RNA guide including a direct repeat sequence and a spacer sequence capable of hybridizing to a target nucleic acid, or a nucleic acid encoding the RNA guide.

In some embodiments of any of the cells described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 601.

In some embodiments of any of the cells described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 601-682.

In some embodiments of any of the cells described herein, the CRISPR-associated protein is capable of recognizing a PAM sequence including a nucleic acid sequence set forth as 5′-GTN-3′.

In some embodiments of any of the cells described herein, the direct repeat sequence includes a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 683-734 or 751-802.

In some embodiments of any of the cells described herein, the spacer sequence includes between about 15 nucleotides to about 50 nucleotides. In some embodiments of any of the cells described herein, the spacer sequence includes between 20 and 44 nucleotides.

In another aspect, the disclosure provides methods of modifying a target nucleic acid, the method including delivering to the target nucleic acid an engineered, non-naturally occurring CRISPR-Cas system including: a CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 601-682; and an RNA guide including a direct repeat sequence and a spacer sequence capable of hybridizing to the target nucleic acid; wherein the CRISPR-associated protein is capable of binding to the RNA guide; and wherein recognition of the target nucleic acid by the CRISPR-associated protein and RNA guide results in a modification of the target nucleic acid. In another aspect, the disclosure provides methods of modifying a target nucleic acid, the method including delivering to the target nucleic acid an engineered, non-naturally occurring CRISPR-Cas system including: a CRISPR-associated protein or a nucleic acid encoding the CRISPR-associated protein, wherein the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 601-682; and an RNA guide including a direct repeat sequence and a spacer sequence capable of hybridizing to the target nucleic acid, or a nucleic acid encoding the RNA guide; wherein the CRISPR-associated protein is capable of binding to the RNA guide; and wherein recognition of the target nucleic acid by the CRISPR-associated protein and RNA guide results in a modification of the target nucleic acid.

In some embodiments of any of the methods described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 601.

In some embodiments of any of the methods described herein, the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 601-682.

In some embodiments of any of the methods described herein, the CRISPR-associated protein is capable of recognizing a PAM sequence including a nucleic acid sequence set forth as 5′-GTN-3′.

In some embodiments of any of the methods described herein, the direct repeat sequence includes a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 683-734 or 751-802.

In some aspects, the present disclosure provides a method of introducing an insertion or deletion into a target nucleic acid in a mammalian cell, comprising a transfection of: (a) a nucleic acid sequence encoding a CRISPR-associated protein described herein, e.g., wherein the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 1-50, 101-145, 301-341, 501-521, or 601-682; and (b) an RNA guide (or a nucleic acid encoding the RNA guide) comprising a direct repeat sequence and a spacer sequence capable of hybridizing to the target nucleic acid, e.g., an RNA guide described herein; wherein the CRISPR-associated protein is capable of binding to the RNA guide; and wherein recognition of the target nucleic acid by the CRISPR-associated protein and RNA guide results in a modification of the target nucleic acid. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 1, 101, 301, 501, or 601. In some embodiments, the CRISPR-associated protein comprises an amino acid sequence of one of any one of SEQ ID NOs: 1, 101, 301, 501, or 601. In some embodiments, the transfection is a transient transfection. In some embodiments, the cell is a human cell.

In some embodiments of any of the methods described herein, the spacer sequence includes between about 15 nucleotides to about 50 nucleotides. In some embodiments of any of the methods described herein, the spacer sequence includes between 20 and 44 nucleotides.

In some embodiments of any of the systems described herein, the CRISPR-associated protein includes at least one (e.g., one, two, or three) RuvC domain or at least one split RuvC domain.

In some embodiments of any of the systems described herein, the CRISPR-associated protein comprises a catalytic residue (e.g., aspartic acid or glutamic acid). In some embodiments of any of the systems described herein, the CRISPR-associated protein cleaves the target nucleic acid. In some embodiments of any of the systems described herein, the CRISPR-associated protein further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

In some embodiments of any of the systems described herein, the nucleic acid encoding the CRISPR-associated protein is codon-optimized for expression in a cell, e.g., a eukaryotic cell, e.g., a mammalian cell, e.g., a human cell. In some embodiments of any of the systems described herein, the nucleic acid encoding the CRISPR-associated protein is operably linked to a promoter. In some embodiments of any of the systems described herein, the nucleic acid encoding the CRISPR-associated protein is in a vector. In some embodiments, the vector comprises a retroviral vector, a lentiviral vector, a phage vector, an adenoviral vector, an adeno-associated vector, or a herpes simplex vector.

In some embodiments of any of the systems described herein, the target nucleic acid is a DNA molecule. In some embodiments of any of the systems described herein, the target nucleic acid includes a PAM sequence.

In some embodiments of any of the systems described herein, the CRISPR-associated protein has non-specific nuclease activity.

In some embodiments of any of the systems described herein, recognition of the target nucleic acid by the CRISPR-associated protein and RNA guide results in a modification of the target nucleic acid. In some embodiments of any of the systems described herein, the modification of the target nucleic acid is a double-stranded cleavage event. In some embodiments of any of the systems described herein, the modification of the target nucleic acid is a single-stranded cleavage event. In some embodiments of any of the systems described herein, the modification of the target nucleic acid results in an insertion event. In some embodiments of any of the systems described herein, the modification of the target nucleic acid results in a deletion event. In some embodiments of any of the systems described herein, the modification of the target nucleic acid results in cell toxicity or cell death.

In some embodiments of any of the systems described herein, the system further includes a donor template nucleic acid. In some embodiments of any of the systems described herein, the donor template nucleic acid is a DNA molecule. In some embodiments of any of the systems described herein, wherein the donor template nucleic acid is an RNA molecule.

In some embodiments of any of the systems described herein, the system does not include a tracrRNA. In some embodiments of any of the systems described herein, the CRISPR-associated protein is self-processing.

In some embodiments of any of the systems described herein, the system further includes a tracrRNA. In some embodiments of any of the systems described herein, the system further includes a modulator RNA.

In some embodiments of any of the systems described herein, the system is present in a delivery composition comprising a nanoparticle, a liposome, an exosome, a microvesicle, or a gene-gun.

In some embodiments of any of the systems described herein, the systems are within a cell. In some embodiments, the cell is a eukaryotic cell. In some embodiments, the cell is a mammalian cell. In some embodiments, the cell is a human cell. In some embodiments, the cell is a prokaryotic cell.

In some embodiments of any of the cells described herein, the CRISPR-associated protein includes at least one (e.g., one, two, or three) RuvC domain or at least one split RuvC domain.

In some embodiments of any of the cells described herein, the CRISPR-associated protein comprises a catalytic residue (e.g., aspartic acid or glutamic acid). In some embodiments of any of the cells described herein, the CRISPR-associated protein cleaves the target nucleic acid. In some embodiments of any of the cells described herein, the CRISPR-associated protein further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

In some embodiments of any of the cells described herein, the nucleic acid encoding the CRISPR-associated protein is codon-optimized for expression in a cell, e.g., a eukaryotic cell, e.g., a mammalian cell, e.g., a human cell. In some embodiments of any of the cells described herein, the nucleic acid encoding the CRISPR-associated protein is operably linked to a promoter. In some embodiments of any of the cells described herein, the nucleic acid encoding the CRISPR-associated protein is in a vector. In some embodiments, the vector comprises a retroviral vector, a lentiviral vector, a phage vector, an adenoviral vector, an adeno-associated vector, or a herpes simplex vector.

In some embodiments of any of the cells described herein, the cell does not include a tracrRNA. In some embodiments of any of the cells described herein, the cell optionally includes a tracrRNA. In some embodiments of any of the cells described herein, the CRISPR-associated protein is self-processing.

In some embodiments of any of the cells described herein, the cell further includes a tracrRNA. In some embodiments of any of the cells described herein, the cell further includes a modulator RNA.

In some embodiments of any of the cells described herein, the cell is a eukaryotic cell. In some embodiments of any of the cells described herein, the cell is a mammalian cell. In some embodiments of any of the cells described herein, the cell is a human cell. In some embodiments of any of the cells described herein, the cell is a prokaryotic cell. In some embodiments, of any of the cells described herein, the cell is a genetically engineered cell.

In some embodiments of any of the cells described herein, the target nucleic acid is a DNA molecule. In some embodiments of any of the cells described herein, the target nucleic acid includes a PAM sequence.

In some embodiments of any of the cells described herein, the CRISPR-associated protein has non-specific nuclease activity.

In some embodiments of any of the cells described herein, recognition of the target nucleic acid by the CRISPR-associated protein and RNA guide results in a modification of the target nucleic acid. In some embodiments of any of the cells described herein, the modification of the target nucleic acid is a double-stranded cleavage event. In some embodiments of any of the cells described herein, the modification of the target nucleic acid is a single-stranded cleavage event. In some embodiments of any of the cells described herein, the modification of the target nucleic acid results in an insertion event. In some embodiments of any of the cells described herein, the modification of the target nucleic acid results in a deletion event. In some embodiments of any of the cells described herein, the modification of the target nucleic acid results in cell toxicity or cell death.

In another aspect, the disclosure provides a method of binding a system described herein to a target nucleic acid in a cell comprising: (a) providing the system; and (b) delivering the system to the cell, wherein the cell comprises the target nucleic acid, wherein the CRISPR-associated protein binds to the RNA guide, and wherein the spacer sequence binds to the target nucleic acid. In some embodiments, the cell is a eukaryotic cell, e.g., a mammalian cell, e.g., a human cell.

In some embodiments of any of the methods described herein, the CRISPR-associated protein comprises a catalytic residue (e.g., aspartic acid or glutamic acid). In some embodiments of any of the methods described herein, the CRISPR-associated protein cleaves the target nucleic acid. In some embodiments of any of the methods described herein, the CRISPR-associated protein further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

In some embodiments of any of the methods described herein, the nucleic acid encoding the CRISPR-associated protein is codon-optimized for expression in a cell, e.g., a eukaryotic cell, e.g., a mammalian cell, e.g., a human cell. In some embodiments, of any of the methods described herein, the cell is a genetically engineered cell. In some embodiments of any of the methods described herein, the nucleic acid encoding the CRISPR-associated protein is operably linked to a promoter. In some embodiments of any of the methods described herein, the nucleic acid encoding the CRISPR-associated protein is in a vector. In some embodiments, the vector comprises a retroviral vector, a lentiviral vector, a phage vector, an adenoviral vector, an adeno-associated vector, or a herpes simplex vector.

In some embodiments of any of the methods described herein, the system does not include a tracrRNA. In some embodiments of any of the methods described herein, the cell optionally includes a tracrRNA.

In some embodiments of any of the methods described herein, the RNA guide optionally includes a tracrRNA and/or a modulator RNA. In some embodiments of any of the methods described herein, the system further includes a tracrRNA. In some embodiments of any of the methods described herein, the system further includes a modulator RNA.

In some embodiments of any of the methods described herein, the target nucleic acid is a DNA molecule. In some embodiments of any of the methods described herein, the target nucleic acid includes a PAM sequence.

In some embodiments of any of the methods described herein, the CRISPR-associated protein has non-specific nuclease activity.

In some embodiments of any of the methods described herein, the modification of the target nucleic acid is a double-stranded cleavage event. In some embodiments of any of the methods described herein, the modification of the target nucleic acid is a single-stranded cleavage event. In some embodiments of any of the methods described herein, the modification of the target nucleic acid results in an insertion event. In some embodiments of any of the methods described herein, the modification of the target nucleic acid results in a deletion event. In some embodiments of any of the methods described herein, the modification of the target nucleic acid results in cell toxicity or cell death.

In another aspect, the disclosure provides a method of editing a target nucleic acid, the method comprising contacting the target nucleic acid with a system described herein. In another aspect, the disclosure provides a method of modifying expression of a target nucleic acid, the method comprising contacting the target nucleic acid with a system described herein. In another aspect, the disclosure provides a method of targeting the insertion of a payload nucleic acid at a site of a target nucleic acid, the method comprising contacting the target nucleic acid with a system described herein. In another aspect, the disclosure provides a method of targeting the excision of a payload nucleic acid from a site at a target nucleic acid, the method comprising contacting the target nucleic acid with a system described herein. In another aspect, the disclosure provides a method of non-specifically degrading single-stranded DNA upon recognition of a DNA target nucleic acid, the method comprising contacting the target nucleic acid with a system described herein.

In another aspect, the disclosure provides a method of detecting a target nucleic acid in a sample, the method comprising: (a) contacting the sample with a system described herein and a labeled reporter nucleic acid, wherein hybridization of the spacer sequence to the target nucleic acid causes cleavage of the labeled reporter nucleic acid; and (b) measuring a detectable signal produced by cleavage of the labeled reporter nucleic acid, thereby detecting the presence of the target nucleic acid in the sample.

In some embodiments of any of the systems or methods provided herein, the contacting comprises directly contacting or indirectly contacting. In some embodiments of any of the systems or methods provided herein, contacting indirectly comprises administering one or more nucleic acids encoding an RNA guide or CRISPR-associated protein described herein under conditions that allow for production of the RNA guide and/or CRISPR-related protein. In some embodiments of any of the systems or methods provided herein, contacting includes contacting in vivo or contacting in vitro. In some embodiments of any of the systems or methods provided herein, contacting a target nucleic acid with the system comprises contacting a cell comprising the nucleic acid with the system under conditions that allow the CRISPR-related protein and guide RNA to reach the target nucleic acid. In some embodiments of any of the systems or methods provided herein, contacting a cell in vivo with the system comprises administering the system to the subject that comprises the cell, under conditions that allow the CRISPR-related protein and guide RNA to reach the cell or be produced in the cell.

In another aspect, the disclosure provides a system provided herein for use in an in vitro or ex vivo method of: (a) targeting and editing a target nucleic acid; (b) non-specifically degrading a single-stranded nucleic acid upon recognition of the nucleic acid; (c) targeting and nicking a non-spacer complementary strand of a double-stranded target upon recognition of a spacer complementary strand of the double-stranded target; (d) targeting and cleaving a double-stranded target nucleic acid; (e) detecting a target nucleic acid in a sample; (f) specifically editing a double-stranded nucleic acid; (g) base editing a double-stranded nucleic acid; (h) inducing genotype-specific or transcriptional-state-specific cell death or dormancy in a cell; (i) creating an indel in a double-stranded nucleic acid target; (j) inserting a sequence into a double-stranded nucleic acid target; or (k) deleting or inverting a sequence in a double-stranded nucleic acid target.

In some aspects, the present disclosure provides a method of detecting a target nucleic acid in a sample, wherein the method comprises contacting the sample with a system described herein and a labeled reporter nucleic acid, wherein hybridization of the crRNA to the target nucleic acid causes cleavage of the labeled reporter nuclei acid, and measuring a detectable signal produced by cleavage of the labeled reporter nucleic acid, thereby detecting the presence of the target nucleic acid in the sample.

The effectors described herein provide additional features that include, but are not limited to, 1) novel nucleic acid editing properties and control mechanisms, 2) smaller size for greater versatility in delivery strategies, 3) genotype triggered cellular processes such as cell death, and 4) programmable RNA-guided DNA insertion, excision, and mobilization, and 5) differentiated profile of pre-existing immunity through a non-human commensal source. See, e.g., Examples 1-15 and FIGS. 3-44. Addition of the novel DNA-targeting systems described herein to the toolbox of techniques for genome and epigenome manipulation enables broad applications for specific, programmed perturbations.

Other features and advantages of the invention will be apparent from the following detailed description and from the claims.

### BRIEF FIGURE DESCRIPTION

The figures are a series of schematics that represent the results of analysis of a protein cluster referred to as CLUST.133120.

FIG. 1A is a schematic representation of the components of the in vivo negative selection screening assay described in Examples 2, 5, 10, 12, and 14. CRISPR array libraries were designed including non-representative spacers uniformly sampled from both strands of the pACYC184 or E. coli essential genes flanked by two DRs and expressed by J23119.

FIG. 1B is a schematic representation of the in vivo negative selection screening workflow described in Example 2. CRISPR array libraries were cloned into the effector plasmid. The effector plasmid and the non-coding plasmid were transformed into E. coli followed by outgrowth for negative selection of CRISPR arrays conferring interference against transcripts from pACYC184 or E. coli essential genes. Targeted sequencing of the effector plasmid was used to identify depleted CRISPR arrays. Small RNAseq was further performed to identify mature crRNAs and potential tracrRNA requirements.

FIG. 2 is a schematic showing the RuvC and Zn finger domains of CLUST.133120 effectors, which is based upon the consensus sequence of the sequences shown in Table 3.

FIG. 3 is a graph for CLUST.133120 3300027740 (effector set forth in SEQ ID NO: 1) showing the degree of depletion activity of the engineered compositions for spacers targeting pACYC184 and direct repeat transcriptional orientations. The degree of depletion with the direct repeat in the “forward” orientation (5′-CCAA . . . CGAC-[spacer]-3′) and with the direct repeat in the “reverse” orientation (5′-GTCG . . . TTGG-[spacer]-3′) are depicted.

FIG. 4A is a graphical representation showing the density of depleted and non-depleted targets for CLUST.133120 3300027740 by location on the pACYC184 plasmid. FIG. 4B is a graphic representation showing the density of depleted and non-depleted targets for CLUST.133120 3300027740 by location on the E. coli strain, E. Cloni. Targets on the top strand and bottom strand are shown separately and in relation to the orientation of the annotated genes. The magnitude of the bands indicates the degree of depletion, wherein the lighter bands are close to the hit threshold of 3. The gradients are heatmaps of RNA sequencing showing relative transcript abundance.

FIG. 5 is a WebLogo of the sequences flanking depleted targets in E. Cloni as a prediction of the PAM sequence for CLUST.133120 3300027740.

FIG. 6 is a graph for CLUST.133120 3300017971 (effector set forth in SEQ ID NO: 2) showing the degree of depletion activity of the engineered compositions for spacers targeting pACYC184 and direct repeat transcriptional orientations. The degree of depletion with the direct repeat in the “forward” orientation (5′-GTCG . . . TACC-[spacer]-3′) and with the direct repeat in the “reverse” orientation (5′-GGTA . . . CGAC-[spacer]-3′) are depicted.

FIG. 7A is a graphical representation showing the density of depleted and non-depleted targets for CLUST.133120 3300017971 by location on the pACYC184 plasmid. FIG. 7B is a graphic representation showing the density of depleted and non-depleted targets for CLUST.133120 3300017971 by location on the E. coli strain, E. Cloni. Targets on the top strand and bottom strand are shown separately and in relation to the orientation of the annotated genes. The magnitude of the bands indicates the degree of depletion, wherein the lighter bands are close to the hit threshold of 3. The gradients are heatmaps of RNA sequencing showing relative transcript abundance.

FIG. 8 is a WebLogo of the sequences flanking depleted targets in E. Cloni as a prediction of the PAM sequence for CLUST.133120 3300017971.

FIG. 9 is a graph for CLUST.133120 3300027740 (effector set forth in SEQ ID NO: 1) showing the degree of depletion activity of the engineered compositions for spacers targeting pACYC184 and direct repeat transcriptional orientations, without a non-coding sequence. The degree of depletion with the direct repeat in the “forward” orientation (5′-CCAA . . . CGAC-[spacer]-3′) and with the direct repeat in the “reverse” orientation (5′-GTCG . . . TTGG-[spacer]-3′) are depicted.

FIG. 10A is a graphical representation showing the density of depleted and non-depleted targets for CLUST.133120 3300027740, without a non-coding sequence, by location on the pACYC184 plasmid. FIG. 10B is a graphic representation showing the density of depleted and non-depleted targets for CLUST.133120 3300027740, without a non-coding sequence, by location on the E. coli strain E. Cloni. Targets on the top strand and bottom strand are shown separately and in relation to the orientation of the annotated genes. The magnitude of the bands indicates the degree of depletion, wherein the lighter bands are close to the hit threshold of 3. The gradients are heatmaps of RNA sequencing showing relative transcript abundance.

FIG. 11 is a WebLogo of the sequences flanking depleted targets in E. Cloni as a prediction of the PAM sequence for CLUST.133120 3300027740 (without a non-coding sequence).

FIG. 12 is a graph for CLUST.133120 3300017971 (effector set forth in SEQ ID NO: 2) showing the degree of depletion activity of the engineered compositions for spacers targeting pACYC184 and direct repeat transcriptional orientations, without a non-coding sequence. The degree of depletion with the direct repeat in the “forward” orientation (5′-GTCG . . . TACC-[spacer]-3′) and with the direct repeat in the “reverse” orientation (5′-GGTA . . . CGAC-[spacer]-3′) are depicted.

FIG. 13A is a graphical representation showing the density of depleted and non-depleted targets for CLUST.133120 3300017971, without a non-coding sequence, by location on the pACYC184 plasmid. FIG. 13B is a graphic representation showing the density of depleted and non-depleted targets for CLUST.133120 3300017971, without a non-coding sequence, by location on the E. coli strain E. Cloni. Targets on the top strand and bottom strand are shown separately and in relation to the orientation of the annotated genes. The magnitude of the bands indicates the degree of depletion, wherein the lighter bands are close to the hit threshold of 3. The gradients are heatmaps of RNA sequencing showing relative transcript abundance.

FIG. 14 is a WebLogo of the sequences flanking depleted targets in E. Cloni as a prediction of the PAM sequence for CLUST.133120 3300017971 (without a non-coding sequence).

FIG. 15A is a schematic of the fluorescence depletion assay described in Example 3 to measure CLUST.133120 effector activity. FIG. 15B shows plots of GFP Depletion Ratios (Non-target/target) for the effector of SEQ ID NO: 1 for Target 1 (SEQ ID NO: 82), Target 2 (SEQ ID NO: 83), and Target 3 (SEQ ID NO: 84).

FIG. 16 is a schematic showing the RuvC domains of CLUST.099129 effectors, which is based upon the consensus sequence of the sequences shown in Table 10.

FIG. 17 is a graph for CLUST.099129 SRR6837557 (effector set forth in SEQ ID NO: 101) showing the degree of depletion activity of the engineered compositions for spacers targeting pACYC184 and direct repeat transcriptional orientations, with a non-coding sequence. The degree of depletion with the direct repeat in the “forward” orientation (5′-GTTT . . . GACC-[spacer]-3′) and with the direct repeat in the “reverse” orientation (5′-AGTC . . . AAAC-[spacer]-3′) are depicted.

FIG. 18A is a graphical representation showing the density of depleted and non-depleted targets for CLUST.099129 SRR6837557, with a non-coding sequence, by location on the pACYC184 plasmid. FIG. 18B is a graphic representation showing the density of depleted and non-depleted targets for CLUST.099129 SRR6837557, with a non-coding sequence, by location on the E. coli strain, E. Cloni. Targets on the top strand and bottom strand are shown separately and in relation to the orientation of the annotated genes. The magnitude of the bands indicates the degree of depletion, wherein the lighter bands are close to the hit threshold of 3. The gradients are heatmaps of RNA sequencing showing relative transcript abundance.

FIG. 19 is a WebLogo of the sequences flanking depleted targets in E. Cloni as a prediction of the PAM sequence for CLUST.099129 SRR6837557 (with a non-coding sequence).

FIG. 20 is a graph for CLUST.099129 SRR6837557 (effector set forth in SEQ ID NO: 101) showing the degree of depletion activity of the engineered compositions for spacers targeting pACYC184 and direct repeat transcriptional orientations, without a non-coding sequence. The degree of depletion with the direct repeat in the “forward” orientation (5′-GTT . . . GACC-[spacer]-3′) and with the direct repeat in the “reverse” orientation (0.5′-AGTC . . . AAAC-[spacer]-3′) are depicted.

FIG. 21A is a graphical representation showing the density of depleted and non-depleted targets for CLUST.099129 SRR6837557, without a non-coding sequence, by location on the pACYC184 plasmid. FIG. 21B is a graphic representation showing the density of depleted and non-depleted targets for CLUST.099129 SRR6837557, without a non-coding sequence. by location on the E. coli strain, E. Cloni. Targets on the top strand and bottom strand are shown separately and in relation to the orientation of the annotated genes. The magnitude of the bands indicates the degree of depletion. wherein the lighter bands are close to the hit threshold of 3. The gradients are heatmaps of RNA sequencing showing relative transcript abundance.

FIG. 22 is a WebLogo of the sequences flanking depleted targets in E. Cloni as a prediction of the PAM sequence for CLUST.099129 SRR6837557 (without a non-coding sequence).

FIG. 23 is a graph for CLUST.099129 3300012971 (effector set forth in SEQ ID NO: 102) showing the degree of depletion activity of the engineered compositions for spacers targeting pACYC184 and direct repeat transcriptional orientations, with a non-coding sequence. The degree of depletion with the direct repeat in the “forward” orientation 5′-GTGC . . . TCAC-[spacer]-3′) and with the direct repeat in the “reverse” orientation (5′-GTGA . . . GCAC-[spacer]-3′) are depicted.

FIG. 24A is a graphical representation showing the density of depleted and non-depleted targets for CLUST.099129 3300012971, with a non-coding sequence, by location on the pACYC184 plasmid. FIG. 24B is a graphic representation showing the density of depleted and non-depleted targets for CLUST.099129 3300012971, with a non-coding sequence, by location on the E. coli strain E. Cloni. Targets on the top strand and bottom strand are shown separately and in relation to the orientation of the annotated genes. The magnitude of the bands indicates the degree of depletion, wherein the lighter bands are close to the hit threshold of 3. The gradients are heatmaps of RNA sequencing showing relative transcript abundance.

FIG. 25 is a WebLogo of the sequences flanking depleted targets in E. Cloni as a prediction of the PAM sequence for CLUST.099129 3300012971 (with a non-coding sequence).

FIG. 26 is a graph for CLUST.099129 3300005764 (effector set forth in SEQ ID NO: 103) showing the degree of depletion activity of the engineered compositions for spacers targeting pACYC184 and direct repeat transcriptional orientations, with a non-coding sequence. The degree of depletion with the direct repeat in the “forward” orientation (5′-GTGC . . . TACT-[spacer]-3′) and with the direct repeat in the “reverse” orientation (5′-AGTA . . . GCAC-[spacer]-3′) are depicted.

FIG. 27A is a graphical representation showing the density of depleted and non-depleted targets for CLUST.099129 3300005764, with a non-coding sequence, by location on the pACYC184 plasmid. FIG. 27B is a graphic representation showing the density of depleted and non-depleted targets for CLUST.099129 3300005764, with a non-coding sequence, by location on the E. coli strain E. Cloni. Targets on the top strand and bottom strand are shown separately and in relation to the orientation of the annotated genes. The magnitude of the bands indicates the degree of depletion, wherein the lighter bands are close to the hit threshold of 3. The gradients are heatmaps of RNA sequencing showing relative transcript abundance.

FIG. 28 is a WebLogo of the sequences flanking depleted targets in E. Cloni as a prediction of the PAM sequence for CLUST.099129 3300005764 (with a non-coding sequence).

FIG. 29A is a schematic of the fluorescence depletion assay (FDA) described in Example 6 to measure CLUST.099129 effector activity. FIG. 29B shows plots of GFP Depletion Ratios (Non-target/target) for the effector of SEQ ID NO: 101 for Target 1 (SEQ ID NO: 175), Target 2 (SEQ ID NO: 176), Target 3 (SEQ ID NO: 177), Target 4 (SEQ ID NO: 178), and Target 5 (SEQ ID NO: 179).

FIG. 30A, FIG. 30B, and FIG. 30C are schematics showing the RuvC and Zn finger domains of CLUST.342201 effectors, which are based upon the consensus sequence of the sequences shown in Table 17.

FIG. 31 is a graph for CLUST.342201 3300006417 (effector set forth in SEQ ID NO: 301) showing the degree of depletion activity of the engineered compositions for spacers targeting pACYC184 and direct repeat transcriptional orientations, with a non-coding sequence. The degree of depletion with the direct repeat in the “forward” orientation (5′-CCAT . . . GAAC-[spacer]-3′) and with the direct repeat in the “reverse” orientation (5′-GTTC . . . ATGG-[spacer]-3′) are depicted.

FIG. 32A is a graphical representation showing the density of depleted and non-depleted targets for CLUST.342201 3300006417, with a non-coding sequence, by location on the pACYC184 plasmid. FIG. 32B is a graphic representation showing the density of depleted and non-depleted targets for CLUST.342201 3300006417, with a non-coding sequence, by location on the E. coli strain, E. Cloni. Targets on the top strand and bottom strand are shown separately and in relation to the orientation of the annotated genes. The magnitude of the bands indicates the degree of depletion, wherein the lighter bands are close to the hit threshold of 3. The gradients are heatmaps of RNA sequencing showing relative transcript abundance.

FIG. 33 is a WebLogo of the sequences flanking depleted targets in E. Cloni as a prediction of the PAM sequence for CLUST.342201 3300006417 (with a non-coding sequence).

FIG. 34 is a schematic showing the RuvC and Zn finger domains of CLUST.195009 effectors, which is based upon the consensus sequence of the sequences shown in TABLE 23.

FIG. 35 is a graph for CLUST.195009 SRR6201554 (effector set forth in SEQ ID NO: 501) showing the degree of depletion activity of the engineered compositions for spacers targeting pACYC184 and direct repeat transcriptional orientations, with a non-coding sequence. The degree of depletion with the direct repeat in the “forward” orientation (5′-CCAG . . . CGAC-[spacer]-3′) and with the direct repeat in the “reverse” orientation (5′-GTCG . . . CTGG-[spacer]-3′) are depicted.

FIG. 36A is a graphical representation showing the density of depleted and non-depleted targets for CLUST.195009 SRR6201554, with a non-coding sequence, by location on the pACYC184 plasmid. FIG. 36B is a graphic representation showing the density of depleted and non-depleted targets for CLUST.195009 SRR6201554, with a non-coding sequence, by location on the E. coli strain, E. Cloni. Targets on the top strand and bottom strand are shown separately and in relation to the orientation of the annotated genes. The magnitude of the bands indicates the degree of depletion, wherein the lighter bands are close to the hit threshold of 3. The gradients are heatmaps of RNA sequencing showing relative transcript abundance.

FIG. 37 is a WebLogo of the sequences flanking depleted targets in E. Cloni as a prediction of the PAM sequence for CLUST.195009 SRR6201554 (with a non-coding sequence).

FIG. 38 is a graph for CLUST.195009 SRR6201554 (effector set forth in SEQ ID NO: 501) showing the degree of depletion activity of the engineered compositions for spacers targeting pACYC184 and direct repeat transcriptional orientations, without a non-coding sequence. The degree of depletion with the direct repeat in the “forward” orientation (5′-CCAG . . . CGAC-[spacer]-3′) and with the direct repeat in the “reverse” orientation (5′-GTCG . . . CTGG-[spacer]-3′) are depicted.

FIG. 39A is a graphical representation showing the density of depleted and non-depleted targets for CLUST.195009 SRR6201554, without a non-coding sequence, by location on the pACYC184 plasmid. FIG. 39B is a graphic representation showing the density of depleted and non-depleted targets for CLUST.195009 SRR6201554, without a non-coding sequence, by location on the E. coli strain, E. Cloni. Targets on the top strand and bottom strand are shown separately and in relation to the orientation of the annotated genes. The magnitude of the bands indicates the degree of depletion, wherein the lighter bands are close to the hit threshold of 3. The gradients are heatmaps of RNA sequencing showing relative transcript abundance.

FIG. 40 is a WebLogo of the sequences flanking depleted targets in E. Cloni as a prediction of the PAM sequence for CLUST.195009 SRR6201554 (without a non-coding sequence).

FIG. 41 is a schematic representation showing the RuvC and Zn finger domains of CLUST.057059 effectors, which are based upon the consensus sequence of the sequences shown in Table 29.

FIG. 42 is a graph for CLUST.057059 3300023179 (effector set forth in SEQ ID NO: 601) showing the degree of depletion activity of the engineered compositions for spacers targeting pACYC184 and direct repeat transcriptional orientations, with a non-coding sequence. The degree of depletion with the direct repeat in the “forward” orientation (5′-CTTG . . . AAAC-[spacer]-3′) and with the direct repeat in the “reverse” orientation (5′-GTTT . . . CAAG-[spacer]-3′) are depicted.

FIG. 43A is a graphical representation showing the density of depleted and non-depleted targets for CLUST.057059 3300023179, with a non-coding sequence, by location on the pACYC184 plasmid. FIG. 43B is a graphic representation showing the density of depleted and non-depleted targets for CLUST.057059 3300023179, with a non-coding sequence, by location on the E. coli strain, E. Cloni. Targets on the top strand and bottom strand are shown separately and in relation to the orientation of the annotated genes. The magnitude of the bands indicates the degree of depletion, wherein the lighter bands are close to the hit threshold of 3. The gradients are heatmaps of RNA sequencing showing relative transcript abundance.

FIG. 44 is a WebLogo of the sequences flanking depleted targets in E. Cloni as a prediction of the PAM sequence for CLUST.057059 3300023179 (with a non-coding sequence).

### DETAILED DESCRIPTION

CRISPR-Cas systems, which are naturally diverse, comprise a wide range of activity mechanisms and functional elements that can be harnessed for programmable biotechnologies. In nature, these systems enable efficient defense against foreign DNA and viruses while providing self versus non-self discrimination to avoid self-targeting. In an engineered setting, these systems provide a diverse toolbox of molecular technologies and define the boundaries of the targeting space. The methods described herein have been used to discover additional mechanisms and parameters within single subunit Class 2 effector systems, which expand the capabilities of RNA-programmable nucleic acid manipulation.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. Applicant reserves the right to alternatively claim any disclosed invention using the transitional phrase “comprising,” “consisting essentially of,” or “consisting of,” according to standard practice in patent law.

As used herein, the singular forms “a,” “an,” and “the” include plural referents unless the context clearly dictates otherwise. For example, reference to “a nucleic acid” means one or more nucleic acids.

It is noted that terms like “preferably,” “suitably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention, it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

The term “CRISPR-Cas system,” as used herein, refers to nucleic acids and/or proteins involved in the expression of, or directing the activity of, CRISPR effectors, including sequences encoding CRISPR effectors, RNA guides, and other sequences and transcripts from a CRISPR locus.

The terms “CRISPR-associated protein,” “CRISPR-Cas effector,” “CRISPR effector,” “effector,” “effector protein,” “CRISPR enzyme,” or the like, as used interchangeably herein, refer to a protein that carries out an enzymatic activity or that binds to a target site on a nucleic acid specified by an RNA guide. In some embodiments, a CRISPR effector has endonuclease activity, nickase activity, and/or exonuclease activity.

The terms “RNA guide,” “guide RNA,” “gRNA,” and “guide sequence,” as used herein, refer to any RNA molecule that facilitates the targeting of an effector described herein to a target nucleic acid, such as DNA and/or RNA. Exemplary “RNA guides” include, but are not limited to, crRNAs, as well as crRNAs hybridized to or fused to either tracrRNAs and/or modulator RNAs. In some embodiments, an RNA guide includes both a crRNA and a tracrRNA, either fused into a single RNA molecule or as separate RNA molecules. In some embodiments, an RNA guide includes a crRNA and a modulator RNA, either fused into a single RNA molecule or as separate RNA molecules. In some embodiments, an RNA guide includes a crRNA, a tracrRNA, and a modulator RNA, either fused into a single RNA molecule or as separate RNA molecules.

The terms “CRISPR effector complex,” “effector complex,” or “surveillance complex,” as used herein, refer to a complex containing a CRISPR effector and an RNA guide. A CRISPR effector complex may further comprise one or more accessory proteins. The one or more accessory proteins may be non-catalytic and/or non-target binding.

The term “CRISPR RNA” or “crRNA” as used herein refers to an RNA molecule comprising a guide sequence used by a CRISPR effector to specifically recognize a nucleic acid sequence. Typically, crRNAs contain a sequence that mediates target recognition and a sequence that forms a duplex with a tracrRNA. A crRNA may comprise a sequence that hybridizes to a tracrRNA. In turn, the crRNA: tracrRNA duplex may bind to a CRISPR effector. As used herein, the term “pre-crRNA” refers to an unprocessed RNA molecule comprising a DR-spacer-DR sequence. As used herein, the term “mature crRNA” refers to a processed form of a pre-crRNA; a mature crRNA may comprise a DR-spacer sequence, wherein the DR is a truncated form of the DR of a pre-crRNA and/or the spacer is a truncated form of the spacer of a pre-crRNA. A crRNA “spacer” sequence is complementary to and capable of partially or completely binding to a nucleic acid target sequence.

The terms “trans-activating crRNA” or “tracrRNA,” as used herein, refer to an RNA molecule comprising a sequence that forms a structure and/or sequence motif required for a CRISPR effector to bind to a specified target nucleic acid.

The term “CRISPR array,” as used herein, refers to a nucleic acid (e.g., DNA) segment that comprises CRISPR repeats and spacers, starting with the first nucleotide of the first CRISPR repeat and ending with the last nucleotide of the final (terminal) CRISPR repeat. Typically, each spacer in a CRISPR array is located between two repeats. The terms “CRISPR repeat,” “CRISPR direct repeat,” and “direct repeat,” as used herein, refer to multiple short direct repeating sequences, which show very little or no sequence variation within a CRISPR array.

The term “modulator RNA” as described herein refers to any RNA molecule that modulates (e.g., increases or decreases) an activity of a CRISPR effector or a nucleoprotein complex that includes a CRISPR effector. In some embodiments, a modulator RNA modulates a nuclease activity of a CRISPR effector or a nucleoprotein complex that includes a CRISPR effector.

As used herein, the term “target nucleic acid” refers to a nucleic acid that comprises a nucleotide sequence complementary to the entirety or a part of the spacer in an RNA guide. In some embodiments, the target nucleic acid comprises a gene. In some embodiments, the target nucleic acid comprises a non-coding region (e.g., a promoter). In some embodiments, the target nucleic acid is single-stranded. In some embodiments, the target nucleic acid is double-stranded. A “transcriptionally-active site,” as used herein, refers to a site in a nucleic acid sequence being actively transcribed.

The terms “activated CRISPR effector complex,” “activated CRISPR complex,” and “activated complex,” as used herein, refer to a CRISPR effector complex capable of modifying a target nucleic acid. In some embodiments, an activated CRISPR complex is capable of modifying a target nucleic acid following binding of the activated CRISPR complex to the target nucleic acid. In some embodiments, binding of an activated CRISPR complex to a target nucleic acid results in an additional cleavage event, such as collateral cleavage.

The term “cleavage event,” as used herein, refers to a break in a nucleic acid, such as DNA and/or RNA. In some embodiments, a cleavage event refers to a break in a target nucleic acid created by a nuclease of a CRISPR system described herein. In some embodiments, the cleavage event is a double-stranded DNA break. In some embodiments, the cleavage event is a single-stranded DNA break. In some embodiments, a cleavage event refers to a break in a collateral nucleic acid.

The term “collateral nucleic acid,” as used herein, refers to a nucleic acid substrate that is cleaved non-specifically by an activated CRISPR complex. The term “collateral DNase activity,” as used herein in reference to a CRISPR effector, refers to non-specific DNase activity of an activated CRISPR complex. The term “collateral RNase activity,” as used herein in reference to a CRISPR effector, refers to non-specific RNase activity of an activated CRISPR complex.

The term “donor template nucleic acid,” as used herein, refers to a nucleic acid molecule that can be used to make a templated change to a target sequence or target-proximal sequence after a CRISPR effector described herein has modified the target nucleic acid. In some embodiments, the donor template nucleic acid is a double-stranded nucleic acid. In some embodiments, the donor template nucleic acid is a single-stranded nucleic acid. In some embodiments, the donor template nucleic acid is linear. In some embodiments, the donor template nucleic acid is circular (e.g., a plasmid). In some embodiments, the donor template nucleic acid is an exogenous nucleic acid molecule. In some embodiments, the donor template nucleic acid is an endogenous nucleic acid molecule (e.g., a chromosome).

As used herein, the terms “polynucleotide,” “nucleotide,” “oligonucleotide,” and “nucleic acid” can be used interchangeably to refer to nucleic acid comprising DNA, RNA, derivatives thereof, or combinations thereof. Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Maniatis et al., 1989, MOLECULAR CLONING: A LABORATORY MANUAL, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.)

The term “genetic modification” or “genetic engineering” broadly refers to manipulation of the genome or nucleic acids of a cell. Likewise, the terms “genetically engineered” and “engineered” refer to a cell comprising a manipulated genome or nucleic acids. Methods of genetic modification of include, for example, heterologous gene expression, gene or promoter insertion or deletion, nucleic acid mutation, altered gene expression or inactivation, enzyme engineering, directed evolution, knowledge-based design, random mutagenesis methods, gene shuffling, and codon optimization.

The term “recombinant” indicates that a nucleic acid, protein, or cell is the product of genetic modification, engineering, or recombination. Generally, the term “recombinant” refers to a nucleic acid, protein, or cell that contains or is encoded by genetic material derived from multiple sources. As used herein, the term “recombinant” may also be used to describe a cell that comprises a mutated nucleic acid or protein, including a mutated form of an endogenous nucleic acid or protein. The terms “recombinant cell” and “recombinant host” can be used interchangeably. In some embodiments, a recombinant cell comprises a CRISPR effector disclosed herein. In some embodiments, the CRISPR effector disclosed herein is self-processing. The CRISPR effector can be codon-optimized for expression in the recombinant cell. In some embodiments, a recombinant cell disclosed herein further comprises an RNA guide. In some embodiments, an RNA guide of a recombinant cell disclosed herein comprises a tracrRNA. In some embodiments, an RNA guide of a recombinant cell disclosed herein does not comprise a tracrRNA. In some embodiments, the recombinant cell is a prokaryotic cell, such as an E. coli cell. In some embodiments, the recombinant cell is a eukaryotic cell, such as a mammalian cell, including a human cell.

As used herein, the term “protospacer adjacent motif” or “PAM” refers to a DNA sequence adjacent to a target sequence to which a complex comprising an effector and an RNA guide binds. In some embodiments, a PAM is required for enzyme activity. As used herein, the term “adjacent” includes instances in which an RNA guide of the complex specifically binds, interacts, or associates with a target sequence that is immediately adjacent to a PAM. In such instances, there are no nucleotides between the target sequence and the PAM. The term “adjacent” also includes instances in which there are a small number (e.g., 1, 2, 3, 4, or 5) of nucleotides between the target sequence, to which the targeting moiety binds, and the PAM.

### Identification of CLUST.133120, CLUST.099129, CLUST.342201, CLUST.195009, and CLUST.057059

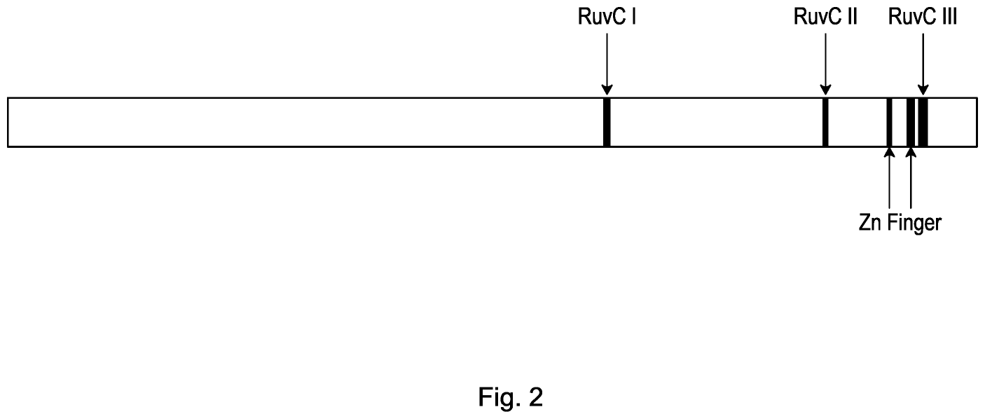

This application relates to the identification, engineering, and use of novel protein families referred to herein as “CLUST.133120”, “CLUST.099129”, “CLUST.342201”, “CLUST.195009”, and “CLUST.057059.” As shown in FIG. 2, the proteins of CLUST.133120 comprise three RuvC domains (denoted RuvC I, RuvC II, and RuvC III) and a Zn finger domain. As shown in TABLE 2, effectors of CLUST.133120 range in size from about 400 amino acids to about 800 amino acids.

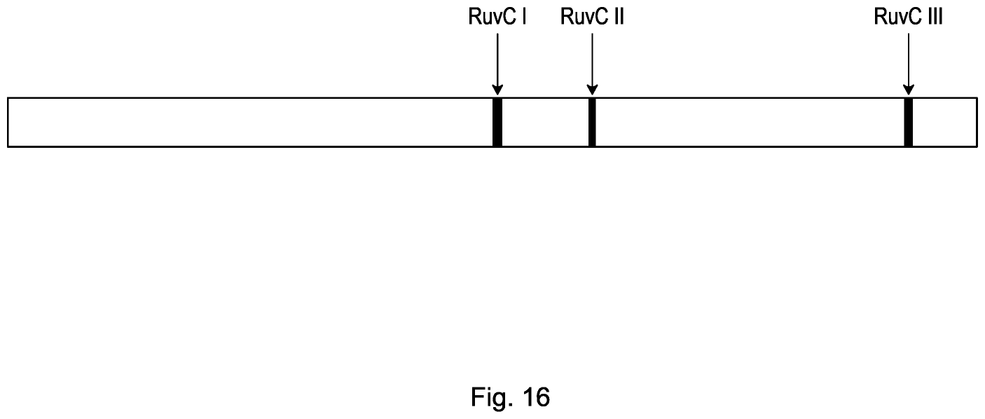

As shown in FIG. 16, the proteins of CLUST.099129 comprise three RuvC domains (denoted RuvC I, RuvC II, and RuvC III). As shown in TABLE 9, effectors of CLUST.099129 range in size from about 500 amino acids to about 700 amino acids.

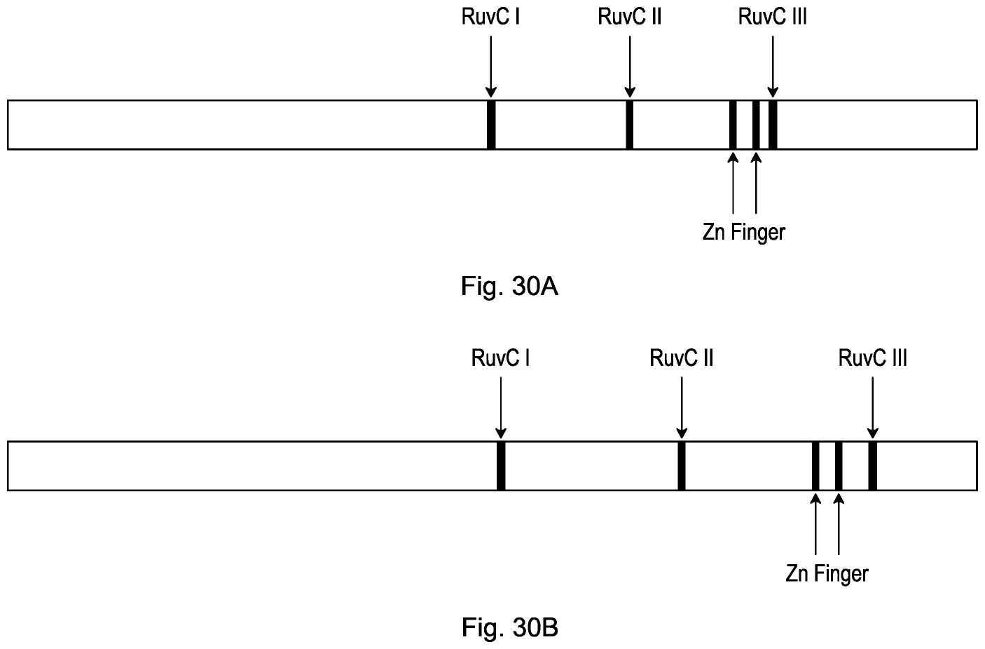

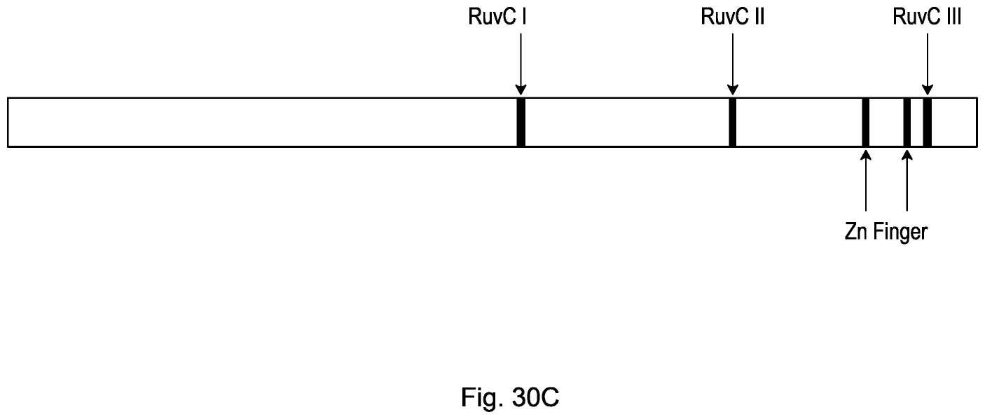

As shown in FIG. 30A, FIG. 30B, and FIG. 30C, the proteins of CLUST.342201 comprise a RuvC domain (denoted RuvC I, RuvC II, and RuvC III) and a Zn finger domain. As shown in TABLE 16. effectors of CLUST.342201 range in size from about 300 to about 650 amino acids. In some embodiments. CLUST.342201 effectors of about 600 amino acids (e.g., effectors having amino acid sequences set forth in SEQ ID NOs: 334-336, 338, or 339) have an architecture as depicted in FIG. 30A. In some embodiments, CLUST.342201 effectors of about 400 amino acids or less than about 400 amino acids have an architecture as depicted in FIG. 30B or FIG. 30C. For example, effectors having sequences set forth in SEQ ID NOs: 302, 303, 308, 309, 310, 311, 316, 324, 325, 330, 331, and 337 may have an architecture as depicted in FIG. 30B, and effectors having sequences set forth in SEQ ID NOs: 301, 304, 306, 307, 312-315, 317-319, 326-329, 332, 333, 340, or 341 may have an architecture as depicted in FIG. 30C. Thus, CLUST.342201 effectors of about 400 amino acids or less than about 400 amino acids have a RuvC III domain at the C-terminus of the effector.

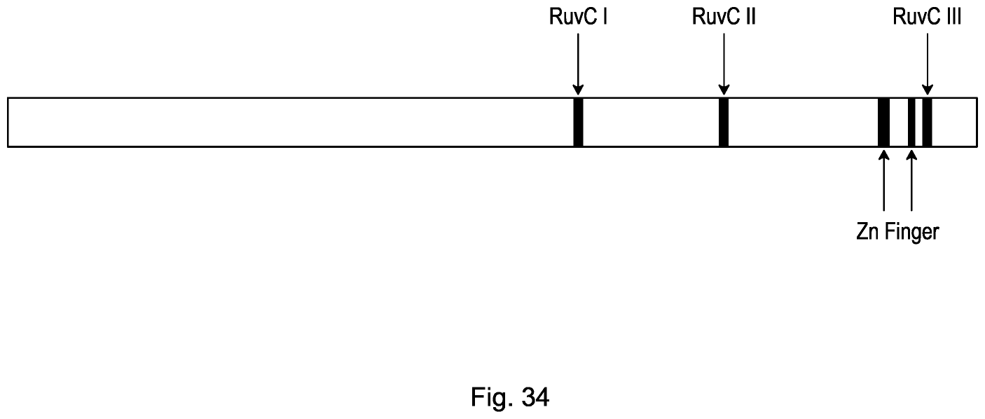

As shown in FIG. 34, the proteins of CLUST.195009 comprise a RuvC domain (denoted RuvC I, RuvC II, and RuvC III) and a Zn finger domain. As shown in TABLE 22, effectors of CLUST.195009 range in size from about 450 amino acids to about 600 amino acids.

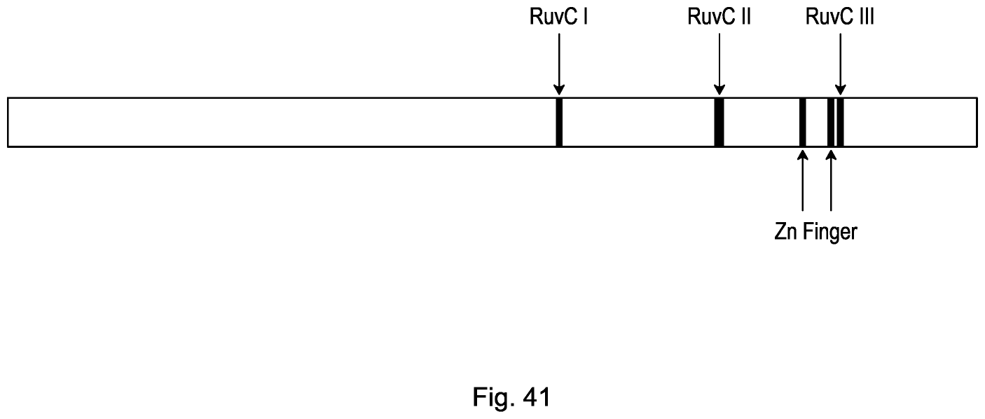

As shown in FIG. 41, the proteins of CLUST.057059 comprise a RuvC domain (denoted RuvC I, RuvC II, and RuvC III) and a Zn finger domain. As shown in TABLE 28, effectors of CLUST.057059 range in size from about 350 to about 700 amino acids.

Therefore, the effectors of CLUST.133120, CLUST.099129, CLUST.342201, CLUST.195009, and CLUST.057059 are significantly smaller than effectors known in the art, as shown below. See, e.g., TABLE 1.

TABLE 1
Sizes of known CRISPR-Cas system effectors.

| Effector | Size (aa) |
|---|---|
| StCas9 | 1128 |
| SpCas9 | 1368 |
| SaCas9 | 1053 |
| FnCpf1 | 1300 |
| AsCpf1 | 1307 |
| LbCpf1 | 1246 |
| C2c1 | 1127 (average) |
| CasX | 982 (average) |
| CasY | 1189 (average) |
| C2c2 | 1232 (average) |

The effectors of CLUST.133120, CLUST.099129, CLUST.342201, CLUST.195009, and CLUST.057059 were identified using computational methods and algorithms to search for and identify proteins exhibiting a strong co-occurrence pattern with certain other features. In certain embodiments, these computational methods were directed to identifying proteins that co-occurred in close proximity to CRISPR arrays. The methods disclosed herein are also useful in identifying proteins that naturally occur within close proximity to other features, both non-coding and protein-coding (e.g., fragments of phage sequences in non-coding areas of bacterial loci or CRISPR Cas1 proteins). It is understood that the methods and calculations described herein may be performed on one or more computing devices.

Sets of genomic sequences were obtained from genomic or metagenomic databases. The databases comprised short reads, or contig level data, or assembled scaffolds, or complete genomic sequences of organisms. Likewise, the databases may comprise genomic sequence data from prokaryotic organisms, or eukaryotic organisms, or may include data from metagenomic environmental samples. Examples of database repositories include the National Center for Biotechnology Information (NCBI) RefSeq, NCBI GenBank, NCBI Whole Genome Shotgun (WGS), and the Joint Genome Institute (JGI) Integrated Microbial Genomes (IMG).

In some embodiments, a minimum size requirement is imposed to select genome sequence data of a specified minimum length. In certain exemplary embodiments, the minimum contig length may be 100 nucleotides, 500 nt, 1 kb, 1.5 kb, 2 kb, 3 kb, 4 kb, 5 kb, 10 kb, 20 kb, 40 kb, or 50 kb.

In some embodiments, known or predicted proteins are extracted from the complete or a selected set of genome sequence data. In some embodiments, known or predicted proteins are taken from extracting coding sequence (CDS) annotations provided by the source database. In some embodiments, predicted proteins are determined by applying a computational method to identify proteins from nucleotide sequences. In some embodiments, the GeneMark Suite is used to predict proteins from genome sequences. In some embodiments, Prodigal is used to predict proteins from genome sequences. In some embodiments, multiple protein prediction algorithms may be used over the same set of sequence data with the resulting set of proteins de-duplicated.

In some embodiments, CRISPR arrays are identified from the genome sequence data. In some embodiments, PILER-CR is used to identify CRISPR arrays. In some embodiments, CRISPR Recognition Tool (CRT) is used to identify CRISPR arrays. In some embodiments, CRISPR arrays are identified by a heuristic that identifies nucleotide motifs repeated a minimum number of times (e.g., 2, 3, or 4 times), where the spacing between consecutive occurrences of a repeated motif does not exceed a specified length (e.g., 50, 100, or 150 nucleotides). In some embodiments, multiple CRISPR array identification tools may be used over the same set of sequence data with the resulting set of CRISPR arrays de-duplicated.

In some embodiments, proteins in close proximity to CRISPR arrays (referred to herein as “CRISPR-proximal protein clusters”) are identified. In some embodiments, proximity is defined as a nucleotide distance, and may be within 20 kb, 15 kb, or 5 kb. In some embodiments, proximity is defined as the number of open reading frames (ORFs) between a protein and a CRISPR array, and certain exemplary distances may be 10, 5, 4, 3, 2, 1, or 0 ORFs. The proteins identified as being within close proximity to a CRISPR array are then grouped into clusters of homologous proteins. In some embodiments, blastclust is used to form CRISPR-proximal protein clusters. In certain other embodiments, mmseqs2 is used to form CRISPR-proximal protein clusters.

To establish a pattern of strong co-occurrence between the members of a CRISPR-proximal protein cluster, a BLAST search of each member of the protein cluster may be performed over the complete set of known and predicted proteins previously compiled. In some embodiments, UBLAST or mmseqs2 may be used to search for similar proteins. In some embodiments, a search may be performed only for a representative subset of proteins in the family.

In some embodiments, the CRISPR-proximal protein clusters are ranked or filtered by a metric to determine co-occurrence. One exemplary metric is the ratio of the number of elements in a protein cluster against the number of BLAST matches up to a certain E value threshold. In some embodiments, a constant E value threshold may be used. In other embodiments, the E value threshold may be determined by the most distant members of the protein cluster. In some embodiments, the global set of proteins is clustered and the co-occurrence metric is the ratio of the number of elements of the CRISPR-proximal protein cluster against the number of elements of the containing global cluster(s).

In some embodiments, a manual review process is used to evaluate the potential functionality and the minimal set of components of an engineered system based on the naturally occurring locus structure of the proteins in the cluster. In some embodiments, a graphical representation of the protein cluster may assist in the manual review and may contain information including pairwise sequence similarity, phylogenetic tree, source organisms/environments, predicted functional domains, and a graphical depiction of locus structures. In some embodiments, the graphical depiction of locus structures may filter for nearby protein families that have a high representation. In some embodiments, representation may be calculated by the ratio of the number of related nearby proteins against the size(s) of the containing global cluster(s). In certain exemplary embodiments, the graphical representation of the protein cluster may contain a depiction of the CRISPR array structures of the naturally occurring loci. In some embodiments, the graphical representation of the protein cluster may contain a depiction of the number of conserved direct repeats versus the length of the putative CRISPR array or the number of unique spacer sequences versus the length of the putative CRISPR array. In some embodiments, the graphical representation of the protein cluster may contain a depiction of various metrics of co-occurrence of the putative effector with CRISPR arrays predict new CRISPR-Cas systems and identify their components.

### Pooled-Screening of CLUST.133120, CLUST.099129, CLUST.342201, CLUST.195009, and CLUST.057059

To efficiently validate the activity, mechanisms, and functional parameters of the engineered CLUST.133120 CRISPR-Cas systems identified herein, a pooled-screening approach in E. coli was used, as described in Example 2.

To efficiently validate the activity, mechanisms, and functional parameters of the engineered CLUST.099129 CRISPR-Cas systems identified herein, a pooled-screening approach in E. coli was used, as described in Example 5.

To efficiently validate the activity, mechanisms, and functional parameters of the engineered CLUST.342201 CRISPR-Cas systems identified herein, a pooled-screening approach in E. coli was used, as described in Example 10.

To efficiently validate the activity, mechanisms, and functional parameters of the engineered CLUST.195009 CRISPR-Cas systems identified herein, a pooled-screening approach in E. coli was used, as described in Example 12.

To efficiently validate the activity, mechanisms, and functional parameters of the engineered CLUST.057059 CRISPR-Cas systems identified herein, a pooled-screening approach in E. coli was used, as described in Example 14.

First, from the computational identification of the conserved protein and noncoding elements of the CLUST.133120, CLUST.099129, CLUST.342201, CLUST.195009, and CLUST.057059 CRISPR-Cas systems, DNA synthesis and molecular cloning were used to assemble the separate components into a single artificial expression vector, which in one embodiment is based on a pET-28a+ backbone. In a second embodiment, the effectors and noncoding elements are transcribed on an mRNA transcript, and different ribosomal binding sites are used to translate individual effectors.

Second, the natural crRNA and targeting spacers were replaced with a library of unprocessed crRNAs containing non-natural spacers targeting a second plasmid, pACYC184. This crRNA library was cloned into the vector backbone comprising the effectors and noncoding elements (e.g., pET-28a+), and the library was subsequently transformed into E. coli along with the pACYC184 plasmid target. Consequently, each resulting E. coli cell contains no more than one targeting array. In an alternate embodiment, the library of unprocessed crRNAs containing non-natural spacers additionally target E. coli essential genes, drawn from resources such as those described in Baba et al. (2006) Mol. Syst. Biol. 2: 2006.0008; and Gerdes et al. (2003) J. Bacteriol. 185(19): 5673-84, the entire contents of each of which are incorporated herein by reference. In this embodiment, positive, targeted activity of the novel CRISPR-Cas systems that disrupts essential gene function results in cell death or growth arrest. In some embodiments, the essential gene targeting spacers can be combined with the pACYC184 targets.

Third, the E. coli were grown under antibiotic selection. In one embodiment, triple antibiotic selection is used: kanamycin for ensuring successful transformation of the pET-28a+ vector containing the engineered CRISPR effector system and chloramphenicol and tetracycline for ensuring successful co-transformation of the pACYC184 target vector. Since pACYC184 normally confers resistance to chloramphenicol and tetracycline, under antibiotic selection, positive activity of the novel CRISPR-Cas system targeting the plasmid will eliminate cells that actively express the effectors, noncoding elements, and specific active elements of the crRNA library. Typically, populations of surviving cells are analyzed 12-14 h post-transformation. In some embodiments, analysis of surviving cells is conducted 6-8 h post-transformation, 8-12 h post-transformation, up to 24 h post-transformation, or more than 24 h post-transformation. Examining the population of surviving cells at a later time point compared to an earlier time point results in a depleted signal compared to the inactive crRNAs.

In some embodiments, double antibiotic selection is used. Withdrawal of either chloramphenicol or tetracycline to remove selective pressure can provide novel information about the targeting substrate, sequence specificity, and potency. For example, cleavage of dsDNA in a selected or unselected gene can result in negative selection in E. coli, wherein depletion of both selected and unselected genes is observed. If the CRISPR-Cas system interferes with transcription or translation (e.g., by binding or by transcript cleavage), then selection will only be observed for targets in the selected resistance gene, rather than in the unselected resistance gene.

In some embodiments, only kanamycin is used to ensure successful transformation of the pET-28a+ vector comprising the engineered CRISPR-Cas system. This embodiment is suitable for libraries containing spacers targeting E. coli essential genes, as no additional selection beyond kanamycin is needed to observe growth alterations. In this embodiment, chloramphenicol and tetracycline dependence is removed, and their targets (if any) in the library provide an additional source of negative or positive information about the targeting substrate, sequence specificity, and potency.

Since the pACYC184 plasmid contains a diverse set of features and sequences that may affect the activity of a CRISPR-Cas system, mapping the active crRNAs from the pooled screen onto pACYC184 provides patterns of activity that can be suggestive of different activity mechanisms and functional parameters. In this way, the features required for reconstituting the novel CRISPR-Cas system in a heterologous prokaryotic species can be more comprehensively tested and studied.

The key advantages of the in vivo pooled-screen described herein include:

(1) Versatility—Plasmid design allows multiple effectors and/or noncoding elements to be expressed; library cloning strategy enables both transcriptional directions of the computationally predicted crRNA to be expressed;

(2) Comprehensive tests of activity mechanisms & functional parameters—Evaluates diverse interference mechanisms, including nucleic acid cleavage; examines co-occurrence of features such as transcription, plasmid DNA replication; and flanking sequences for crRNA library can be used to reliably determine PAMs with complexity equivalence of 4N's;

(3) Sensitivity—pACYC184 is a low copy plasmid, enabling high sensitivity for CRISPR-Cas activity since even modest interference rates can eliminate the antibiotic resistance encoded by the plasmid; and

(4) Efficiency—Optimized molecular biology steps to enable greater speed and throughput RNA-sequencing and protein expression samples can be directly harvested from the surviving cells in the screen.

The novel CLUST.133120, CLUST.099129, CLUST.342201, CLUST.195009, and CLUST.057059 CRISPR-Cas families described herein were evaluated using this in vivo pooled-screen to evaluate their operational elements, mechanisms, and parameters, as well as their ability to be active and reprogrammed in an engineered system outside of its endogenous cellular environment.

### CRISPR Effector Activity and Modifications

In some embodiments, a CRISPR effector of CLUST.133120, CLUST.099129, CLUST.342201, CLUST.195009, or CLUST.057059 and an RNA guide form a “binary” complex that may include other components. The binary complex is activated upon binding to a nucleic acid substrate that is complementary to a spacer sequence in the RNA guide (i.e., a sequence-specific substrate or target nucleic acid). In some embodiments, the sequence-specific substrate is a double-stranded DNA. In some embodiments, the sequence-specific substrate is a single-stranded DNA. In some embodiments, the sequence-specific substrate is a single-stranded RNA. In some embodiments, the sequence-specific substrate is a double-stranded RNA. In some embodiments, the sequence-specificity requires a complete match of the spacer sequence in the RNA guide (e.g., crRNA) to the target substrate. In other embodiments, the sequence specificity requires a partial (contiguous or non-contiguous) match of the spacer sequence in the RNA guide (e.g., crRNA) to the target substrate.

In some embodiments, the binary complex becomes activated upon binding to the target substrate. In some embodiments, the activated complex exhibits “multiple turnover” activity, whereby upon acting on (e.g., cleaving) the target substrate the activated complex remains in an activated state. In some embodiments, the activated binary complex exhibits “single turnover” activity, whereby upon acting on the target substrate the binary complex reverts to an inactive state. In some embodiments, the activated binary complex exhibits non-specific (i.e., “collateral”) cleavage activity whereby the complex cleaves non-target nucleic acids. In some embodiments, the non-target nucleic acid is a DNA molecule (e.g., a single-stranded or a double-stranded DNA). In some embodiments, the non-target nucleic acid is an RNA molecule (e.g., a single-stranded or a double-stranded RNA).

In some embodiments, a CRISPR effector described herein can be fused to one or more peptide tags, including a His-tag, GST-tag, FLAG-tag, or myc-tag. In some embodiments, a CRISPR effector described herein can be fused to a detectable moiety such as a fluorescent protein (e.g., green fluorescent protein or yellow fluorescent protein). In some embodiments, a CRISPR effector and/or accessory protein of this disclosure is fused to a peptide or non-peptide moiety that allows the protein to enter or localize to a tissue, a cell, or a region of a cell. For instance, a CRISPR effector of this disclosure may comprise a nuclear localization sequence (NLS) such as an SV40 (simian virus 40) NLS, c-Myc NLS, or other suitable monopartite NLS. The NLS may be fused to the N-terminus and/or C-terminus of the CRISPR effector, and may be fused singly (i.e., a single NLS) or concatenated (e.g., a chain of 2, 3, 4, etc. NLS).

In some embodiments, at least one Nuclear Export Signal (NES) is attached to a nucleic acid sequences encoding the CRISPR effector. In some embodiments, a C-terminal and/or N-terminal NLS or NES is attached for optimal expression and nuclear targeting in eukaryotic cells, e.g., human cells.

In those embodiments where a tag is fused to a CRISPR effector, such tag may facilitate affinity-based or charge-based purification of the CRISPR effector, e.g., by liquid chromatography or bead separation utilizing an immobilized affinity or ion-exchange reagent. As a non-limiting example, a recombinant CRISPR effector of this disclosure comprises a polyhistidine (His) tag, and for purification is loaded onto a chromatography column comprising an immobilized metal ion (e.g. a Zn²⁺, Ni²⁺, Cu²⁺ ion chelated by a chelating ligand immobilized on the resin, which resin may be an individually prepared resin or a commercially available resin or ready to use column such as the HisTrap FF column commercialized by GE Healthcare Life Sciences, Marlborough, Mass. Following the loading step, the column is optionally rinsed, e.g., using one or more suitable buffer solutions, and the His-tagged protein is then eluted using a suitable elution buffer. Alternatively, or additionally, if the recombinant CRISPR effector of this disclosure utilizes a FLAG-tag, such protein may be purified using immunoprecipitation methods known in the industry. Other suitable purification methods for tagged CRISPR effectors or accessory proteins of this disclosure will be evident to those of skill in the art.

The proteins described herein (e.g., CRISPR effectors or accessory proteins) can be delivered or used as either nucleic acid molecules or polypeptides. When nucleic acid molecules are used, the nucleic acid molecule encoding the CRISPR effector can be codon-optimized. The nucleic acid can be codon optimized for use in any organism of interest, in particular human cells or bacteria. For example, the nucleic acid can be codon-optimized for any non-human eukaryote including mice, rats, rabbits, dogs, livestock, or non-human primates. Codon usage tables are readily available, for example, at the “Codon Usage Database” available at www.kazusa.orjp/codon/and these tables can be adapted in a number of ways. See Nakamura et al. Nucl. Acids Res. 28:292 (2000), which is incorporated herein by reference in its entirety. Computer algorithms for codon optimizing a particular sequence for expression in a particular host cell are also available, such as Gene Forge (Aptagen; Jacobus, Pa.).

In some instances, nucleic acids of this disclosure which encode CRISPR effectors for expression in eukaryotic (e.g., human, or other mammalian cells) cells include one or more introns, i.e., one or more non-coding sequences comprising, at a first end (e.g., a 5′ end), a splice-donor sequence and, at second end (e.g., the 3′ end) a splice acceptor sequence. Any suitable splice donor/splice acceptor can be used in the various embodiments of this disclosure, including without limitation simian virus 40 (SV40) intron, beta-globin intron, and synthetic introns. Alternatively, or additionally, nucleic acids of this disclosure encoding CRISPR effectors or accessory proteins may include, at a 3′ end of a DNA coding sequence, a transcription stop signal such as a polyadenylation (polyA) signal. In some instances, the polyA signal is located in close proximity to, or adjacent to, an intron such as the SV40 intron.

Deactivated/Inactivated CRISPR Effectors

The CRISPR effectors described herein can be modified to have diminished nuclease activity, e.g., nuclease inactivation of at least 50%, at least 60%, at least 70%, at least 80%, at least 90%, at least 95%, at least 97%, or 100% as compared with the wild type CRISPR effectors. The nuclease activity can be diminished by several methods known in the art, e.g., introducing mutations into the nuclease domains of the proteins. In some embodiments, catalytic residues for the nuclease activities are identified, and these amino acid residues can be substituted by different amino acid residues (e.g., glycine or alanine) to diminish the nuclease activity.

The inactivated CRISPR effectors can comprise or be associated with one or more functional domains (e.g., via fusion protein, linker peptides, “GS” linkers, etc.). These functional domains can have various activities, e.g., methylase activity, demethylase activity, transcription activation activity, transcription repression activity, transcription release factor activity, histone modification activity, RNA cleavage activity, DNA cleavage activity, nucleic acid binding activity, and switch activity (e.g., light inducible). In some embodiments, the functional domains are Krappel associated box (KRAB), VP64, VP16, Fok1, P65, HSF1, MyoD1, and biotin-APEX.

The positioning of the one or more functional domains on the inactivated CRISPR effectors is one that allows for correct spatial orientation for the functional domain to affect the target with the attributed functional effect. For example, if the functional domain is a transcription activator (e.g., VP16, VP64, or p65), the transcription activator is placed in a spatial orientation that allows it to affect the transcription of the target. Likewise, a transcription repressor is positioned to affect the transcription of the target, and a nuclease (e.g., Fok1) is positioned to cleave or partially cleave the target. In some embodiments, the functional domain is positioned at the N-terminus of the CRISPR effector. In some embodiments, the functional domain is positioned at the C-terminus of the CRISPR effector. In some embodiments, the inactivated CRISPR effector is modified to comprise a first functional domain at the N-terminus and a second functional domain at the C-terminus.

Split Enzymes

The present disclosure also provides a split version of the CRISPR effectors described herein. The split version of the CRISPR effectors may be advantageous for delivery. In some embodiments, the CRISPR effectors are split to two parts of the enzymes, which together substantially comprises a functioning CRISPR effector.

The split can be done in a way that the catalytic domain(s) are unaffected. The CRISPR effectors may function as a nuclease or may be inactivated enzymes, which are essentially RNA-binding proteins with very little or no catalytic activity (e.g., due to mutation(s) in its catalytic domains).

In some embodiments, the nuclease lobe and α-helical lobe are expressed as separate polypeptides. Although the lobes do not interact on their own, the RNA guide recruits them into a ternary complex that recapitulates the activity of full-length CRISPR effectors and catalyzes site-specific DNA cleavage. The use of a modified RNA guide abrogates split-enzyme activity by preventing dimerization, allowing for the development of an inducible dimerization system. The split enzyme is described, e.g., in Wright et al. “Rational design of a split-Cas9 enzyme complex,” Proc. Natl. Acad. Sci., 112.10 (2015): 2984-2989, which is incorporated herein by reference in its entirety.

In some embodiments, the split enzyme can be fused to a dimerization partner, e.g., by employing rapamycin sensitive dimerization domains. This allows the generation of a chemically inducible CRISPR effector for temporal control of CRISPR effector activity. The CRISPR effector can thus be rendered chemically inducible by being split into two fragments, and rapamycin-sensitive dimerization domains can be used for controlled reassembly of the CRISPR effector.

The split point is typically designed in silico and cloned into the constructs. During this process, mutations can be introduced to the split enzyme and non-functional domains can be removed. In some embodiments, the two parts or fragments of the split CRISPR effector (i.e., the N-terminal and C-terminal fragments) can form a full CRISPR effector, comprising, e.g., at least 70%, at least 80%, at least 90%, at least 95%, or at least 99% of the sequence of the wild-type CRISPR effector.

Self-Activating or Inactivating Enzymes

The CRISPR effectors described herein can be designed to be self-activating or self-inactivating. In some embodiments, the CRISPR effectors are self-inactivating. For example, the target sequence can be introduced into the CRISPR effector coding constructs. Thus, the CRISPR effectors can cleave the target sequence, as well as the construct encoding the enzyme thereby self-inactivating their expression. Methods of constructing a self-inactivating CRISPR system is described, e.g., in Epstein et al., “Engineering a Self-Inactivating CRISPR System for AAV Vectors,” Mol. Ther., 24 (2016): S50, which is incorporated herein by reference in its entirety.

In some other embodiments, an additional RNA guide, expressed under the control of a weak promoter (e.g., 7SK promoter), can target the nucleic acid sequence encoding the CRISPR effector to prevent and/or block its expression (e.g., by preventing the transcription and/or translation of the nucleic acid). The transfection of cells with vectors expressing the CRISPR effector, RNA guides, and RNA guides that target the nucleic acid encoding the CRISPR effector can lead to efficient disruption of the nucleic acid encoding the CRISPR effector and decrease the levels of CRISPR effector, thereby limiting the genome editing activity.

In some embodiments, the genome editing activity of a CRISPR effector can be modulated through endogenous RNA signatures (e.g., miRNA) in mammalian cells. The CRISPR effector switch can be made by using a miRNA-complementary sequence in the 5′-UTR of mRNA encoding the CRISPR effector. The switches selectively and efficiently respond to miRNA in the target cells. Thus, the switches can differentially control the genome editing by sensing endogenous miRNA activities within a heterogeneous cell population. Therefore, the switch systems can provide a framework for cell-type selective genome editing and cell engineering based on intracellular miRNA information (Hirosawa et al. “Cell-type-specific genome editing with a microRNA-responsive CRISPR-Cas9 switch,” Nucl. Acids Res., 2017 Jul. 27; 45(13): e118).

Inducible CRISPR Effectors

The CRISPR effectors can be inducible, e.g., light inducible or chemically inducible. This mechanism allows for activation of the functional domain in a CRISPR effector. Light inducibility can be achieved by various methods known in the art, e.g., by designing a fusion complex wherein CRY2 PHR/CIBN pairing is used in split CRISPR effectors (see, e.g., Konermann et al., “Optical control of mammalian endogenous transcription and epigenetic states,” Nature, 500.7463 (2013): 472). Chemical inducibility can be achieved, e.g., by designing a fusion complex wherein FKBP/FRB (FK506 binding protein/FKBP rapamycin binding domain) pairing is used in split CRISPR effectors. Rapamycin is required for forming the fusion complex, thereby activating the CRISPR effectors (see, e.g., Zetsche et al., “A split-Cas9 architecture for inducible genome editing and transcription modulation,” Nature Biotech., 33.2 (2015): 139-142).

Furthermore, expression of a CRISPR effector can be modulated by inducible promoters, e.g., tetracycline or doxycycline controlled transcriptional activation (Tet-On and Tet-Off expression system), hormone inducible gene expression system (e.g., an ecdysone inducible gene expression system), and an arabinose-inducible gene expression system. When delivered as RNA, expression of the RNA targeting effector protein can be modulated via a riboswitch, which can sense a small molecule like tetracycline (see, e.g., Goldfless et al., “Direct and specific chemical control of eukaryotic translation with a synthetic RNA-protein interaction,” Nucl. Acids Res., 40.9 (2012): e64-e64).

Various embodiments of inducible CRISPR effectors and inducible CRISPR systems are described, e.g., in U.S. Pat. No. 8,871,445, US 20160208243, and WO 2016205764, each of which is incorporated herein by reference in its entirety.

Functional Mutations

Various mutations or modifications can be introduced into a CRISPR effector as described herein to improve specificity and/or robustness. In some embodiments, the amino acid residues that recognize the Protospacer Adjacent Motif (PAM) are identified. The CRISPR effectors described herein can be modified further to recognize different PAMs, e.g., by substituting the amino acid residues that recognize PAM with other amino acid residues.

In some embodiments, the CRISPR effectors can recognize, e.g., 5′-TTN-3′, or 5′-TN-3′ PAM, wherein “N” is any nucleotide.

In some embodiments, the CRISPR effectors can recognize, e.g., 5′-GTN-3′, 5′-TG-3′, 5′-TR-3′, or 5′-RATG-3′, wherein “N” is any nucleotide and “R” is A or G.

In some embodiments, the CRISPR effectors can recognize, e.g., 5′-AAG-3′, 5′-AAD-3′, 5′-AAR-3′, 5′-RAAG-3′ (SEQ ID NO: 921), 5′-RAAR-3′ (SEQ ID NO: 922), 5′-RAAD-3′ (SEQ ID NO: 923), wherein “D” is A, G, or T, and “R” is A or G.

In some embodiments, the CRISPR effectors can recognize, e.g., 5′-TTN-3′, wherein “N” is any nucleotide.

In some embodiments, the CRISPR effectors can recognize, e.g., 5′-GTN-3′, wherein “N” is any nucleotide.

In some embodiments, the CRISPR effectors described herein can be mutated at one or more amino acid residue to modify one or more functional activities. For example, in some embodiments, the CRISPR effector is mutated at one or more amino acid residues to modify its helicase activity. In some embodiments, the CRISPR effector is mutated at one or more amino acid residues to modify its nuclease activity (e.g., endonuclease activity or exonuclease activity). In some embodiments, the CRISPR effector is mutated at one or more amino acid residues to modify its ability to functionally associate with an RNA guide. In some embodiments, the CRISPR effector is mutated at one or more amino acid residues to modify its ability to functionally associate with a target nucleic acid.

In some embodiments, the CRISPR effectors described herein are capable of cleaving a target nucleic acid molecule. In some embodiments, the CRISPR effector cleaves both strands of the target nucleic acid molecule. However, in some embodiments, the CRISPR effector is mutated at one or more amino acid residues to modify its cleaving activity. For example, in some embodiments, the CRISPR effector may comprise one or more mutations that increase the ability of the CRISPR effector to cleave a target nucleic acid. In another example, in some embodiments, the CRISPR effector may comprise one or more mutations that render the enzyme incapable of cleaving a target nucleic acid. In other embodiments, the CRISPR effector may comprise one or more mutations such that the enzyme is capable of cleaving a strand of the target nucleic acid (i.e., nickase activity). In some embodiments, the CRISPR effector is capable of cleaving the strand of the target nucleic acid that is complementary to the strand that the RNA guide hybridizes to. In some embodiments, the CRISPR effector is capable of cleaving the strand of the target nucleic acid that the RNA guide hybridizes to.

In some embodiments, one or more residues of a CRISPR effector disclosed herein are mutated to an arginine moiety. In some embodiments, one or more residues of a CRISPR effector disclosed herein are mutated to a glycine moiety. In some embodiments, one or more residues of a CRISPR effector disclosed herein are mutated based upon consensus residues of a phylogenetic alignment of CRISPR effectors disclosed herein.

In some embodiments, a CRISPR effector described herein may be engineered to comprise a deletion in one or more amino acid residues to reduce the size of the enzyme while retaining one or more desired functional activities (e.g., nuclease activity and the ability to interact functionally with an RNA guide). The truncated CRISPR effector may be used advantageously in combination with delivery systems having load limitations.

In one aspect, the present disclosure provides nucleic acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the nucleic sequences described herein (e.g., in any of SEQ ID NOS: 1-50), while maintaining the domain architecture shown in FIG. 2. In another aspect, the present disclosure also provides amino acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequences described herein, while maintaining the domain architecture shown in FIG. 2.

In one aspect, the present disclosure provides nucleic acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the nucleic sequences described herein (e.g., in any of SEQ ID NOS: 101-145), while maintaining the domain architecture shown in FIG. 16. In another aspect, the present disclosure also provides amino acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequences described herein, while maintaining the domain architecture shown in FIG. 16.

In one aspect, the present disclosure provides nucleic acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the nucleic sequences described herein (e.g., in any of SEQ ID NOS: 301-341), while maintaining the domain architecture shown in FIG. 30A, FIG. 30B, or FIG. 30C. In another aspect, the present disclosure also provides amino acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequences described herein, while maintaining the domain architecture shown in FIG. 30A, FIG. 30B, or FIG. 30C.

In one aspect, the present disclosure provides nucleic acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the nucleic sequences described herein (e.g., in any of SEQ ID NOS: 501-521), while maintaining the domain architecture shown in FIG. 34. In another aspect, the present disclosure also provides amino acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequences described herein, while maintaining the domain architecture shown in FIG. 34.

In one aspect, the present disclosure provides nucleic acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the nucleic sequences described herein (e.g., in any of SEQ ID NOS: 601-682), while maintaining the domain architecture shown in FIG. 41. In another aspect, the present disclosure also provides amino acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequences described herein, while maintaining the domain architecture shown in FIG. 41. In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are the same as the sequences described herein. In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from the sequences described herein.

In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as the sequences described herein. In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from the sequences described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In general, the length of a reference sequence aligned for comparison purposes should be at least 80% of the length of the reference sequence, and in some embodiments at least 90%, 95%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

### RNA Guide and RNA Guide Modifications

In some embodiments, an RNA guide described herein comprises a uracil (U). In some embodiments, an RNA guide described herein comprises a thymine (T). In some embodiments, a direct repeat sequence of an RNA guide described herein comprises a uracil (U). In some embodiments, a direct repeat sequence of an RNA guide described herein comprises a thymine (T). In some embodiments, a direct repeat sequence according to Table 4, 7, 11, 14, 18, 24, 32, 35, or 30 comprises a sequence comprising a uracil, in one or more (e.g., all) places indicated as thymine in the corresponding sequences in Table 4, 7, 11, 14, 18, 24, 32, 35, or 30.

In some embodiments, the direct repeat comprises only one copy of a sequence that is repeated in an endogenous CRISPR array. In some embodiments, the direct repeat is a full-length sequence adjacent to (e.g., flanking) one or more spacer sequences found in an endogenous CRISPR array. In some embodiments, the direct repeat is a portion (e.g., processed portion) of a full-length sequence adjacent to (e.g., flanking) one or more spacer sequences found in an endogenous CRISPR array.

Spacer and Direct Repeat

CLUST.133120

The spacer length of RNA guides can range from about 15 to 55 nucleotides. In some embodiments, the spacer length of an RNA guide is at least 15 nucleotides, at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides, from 15 to 23 nucleotides, from 16 to 22 nucleotides, from 17 to 20 nucleotides, from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 40, or 45 nucleotides), from 30 or 35 to 40 nucleotides, from 41 to 45 nucleotides, from 45 to 50 nucleotides, or longer.

In some embodiments, the direct repeat length of the RNA guide is at least 16 nucleotides, or is from 16 to 20 nucleotides (e.g., 16, 17, 18, 19, or 20 nucleotides). In some embodiments, the direct repeat length of the RNA guide is 19 nucleotides.

Exemplary full-length direct repeat sequences (e.g., direct repeat sequences of pre-crRNAs or unprocessed crRNAs) and direct repeat sequences of mature crRNAs (e.g., direct repeat sequences of processed crRNAs) are shown in Table 32. See also TABLE 4.

TABLE 32
Exemplary direct repeat sequences of pre-crRNA and mature crRNA sequences.

| Effector | pre-crRNA Direct Repeat Sequence | Mature crRNA Direct Repeat Sequence |
|---|---|---|
| 3300027740 (SEQ | CCAACCAATGCCAGCGCGACGG | AATGCCAGCGCGACGGCTTATG |
| ID NO:1) | CTTATGAGTCGCGAC (SEQ ID | AGTCGCGAC (SEQ ID NO: 85) |
| | NO: 51) | |
| 3300017971 | GGTAAAACACCTGCGAGATGGT | AACACCTGCGAGATGGTTTATGA |
| (SEQ ID NO: 2) | TTATGAATCTCGAC (SEQ ID NO: | ATCTCGAC (SEQ ID NO: 86) |
| | 52) | |
| SRR2657585 | CCAACAATGCTAGCGAGACGGC | AATGCTAGCGAGACGGCTTCAT |
| (SEQ ID NO: 16) | TTCATGATCTCGACG (SEQ ID | GATCTCGACG (SEQ ID NO: 87) |
| | NO: 59) | |

In some embodiments, PAMs corresponding to effectors of the present application are set forth as 5′-TTN-3′ and 5′-TN-3′. As used herein, N's can each be any nucleotide (e.g., A, G, T, or C) or a subset thereof (e.g., R (A or G), Y (C or T), K (G or T), B (G, T, or C), H (A, C, or T).

In some embodiments, an RNA guide further comprises a tracrRNA. In some embodiments, the tracrRNA is not required (e.g., the tracrRNA is optional). In some embodiments, the tracrRNA is a portion of the non-coding sequences shown in TABLE 5. For example, in some embodiments, the optional tracrRNA is a sequence of TABLE 33.

TABLE 33
Exemplary tracrRNA sequences.

| Effector | tracrRNA |
|---|---|
| 3300027740 | GAACACCAGCGAGATGGTTTATGAATCTCGACAGGTCGAAGTAGATACC |
| (SEQ ID NO: 1) | AAA (SEQ ID NO: 88) |
| | ATGAAGAGGTAACACCTACGAGACGGTTTATGAGTTTCGACTATACTAC |
| | TGATAATCTGCCCCAACT (SEQ ID NO: 89) |
| | ATGGTTTATAAATCTCGACAGACTCGCGGATGGAAGAGAACTCGCGACA |
| | CTCGCGGGGTAGAACACCAGCGAGATGGTTTAT (SEQ ID NO: 90) |
| | GGTTTATAAATCTCGACAGACTCGCGGATGGAAGAGAACTCGCGACACT |
| | CGCGGGGTAGAACACCAGCGAGATGGTT (SEQ ID NO: 91) |
| 3300017971 | AATGCGCAAGATGGCTGCCGAGGTGCTAGGCGAGGAGCTGGAAACGCC |
| (SEQ ID NO: 2) | GACTGGGCGT (SEQ ID NO: 92) |
| | TATGCCAACCCCATTTTTCCAGCCAGGCGATCCGCGTTGCCACCGCGCC |
| | GGCAGACCCAAGGGGACAGCGCTCAAGAAAATGCGCAAGATGGCTGCC |
| | GAGGTGCTAGGCGAGGAGCTGGAAACGCCGACTGGGCGTATGA (SEQ ID |
| | NO: 93) |
| | CTACTATTTATGTAGTAATCATTATGCCAACCCCATTTTTCCAGCCAGGC |
| | GATCCGCGT (SEQ ID NO: 94) |

CLUST.099129

The spacer length of RNA guides can range from about 15 to 55 nucleotides. In some embodiments, the spacer length of an RNA guide is at least 15 nucleotides, at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides, from 15 to 23 nucleotides, from 16 to 22 nucleotides, from 17 to 20 nucleotides, from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 40, or 45 nucleotides), from 30 or 35 to 40 nucleotides, from 41 to 45 nucleotides, from 45 to 50 nucleotides, from 50 to 55 nucleotides, or longer.

In some embodiments, the direct repeat length of the RNA guide is at least 16 nucleotides, or is from 16 to 20 nucleotides (e.g., 16, 17, 18, 19, or 20 nucleotides). In some embodiments, the direct repeat length of the RNA guide is up to 40 nucleotides. See TABLE 11.

Exemplary full-length direct repeat sequences (e.g., direct repeat sequences of pre-crRNAs or unprocessed crRNAs) and direct repeat sequences of mature crRNAs (e.g., direct repeat sequences of processed crRNAs) are shown in Table 7. See also TABLE 11.

TABLE 7
Exemplary direct repeat sequences of pre-crRNA and mature crRNA sequences.

| Effector | pre-crRNA Direct Repeat Sequence | Mature crRNA Direct Repeat Sequence |
|---|---|---|
| SRR6837557 | AGTCGCGAATAACTGTTCAGCGG | ATAACTGTTCAGCGGAGAAGCC |
| (SEQ ID NO: 101) | AGAAGCCGCTGAAAC | GCTG (SEQ ID NO: 181) |
| | (SEQ ID NO: 146) | |
| 3300012971 | GTGATTGCAGACTGGTCGTGCGA | TGGTCGTGCGAATGCGCACGGC |
| (SEQ ID NO: 102) | ATGCGCACGGCAC (SEQ ID NO: | AC (SEQ ID NO: 182) |
| | 147) | |
| SRR6266511 | GTGCCGTGCGGATTCGCACGACC | TGGTCGTGCGAATCCGCACGGC |
| (SEQ ID NO: 123) | AACTTTCAATTAC | AC (SEQ ID NO: 183) |
| | (SEQ ID NO: 180) | |
| SRR6837571 | GTCGCGAATAACTGTTCAGCGGA | ATAACTGTTCAGCGGAGAAGCC |
| (SEQ ID NO: 145) | GAAGCCGCTGAAAC (SEQ ID NO: | GCTG (SEQ ID NO: 181) |
| | 162) | |

In some embodiments, PAMs corresponding to effectors of the present application are set forth as 5′-GTN-3′, 5′-TG-3′, 5′-TR-3′, or 5′-RATG-3′. As used herein, N's can each be any nucleotide (e.g., A, G, T, or C) or a subset thereof (e.g., R (A or G), Y (C or T), K (G or T), B (G, T, or C), H (A, C, or T).

In some embodiments, an RNA guide further comprises a tracrRNA. In some embodiments, the tracrRNA is not required (e.g., the tracrRNA is optional). In some embodiments, the tracrRNA is a portion of the non-coding sequences shown in TABLE 12. For example, in some embodiments, the optional tracrRNA is a sequence of TABLE 8.

TABLE 8
Exemplary tracrRNA sequences.

| Effector | tracrRNA Sequence |
|---|---|
| SRR6837557 | TTGGGTTTGTGAAAACGGAAGGGCTGTAA |
| (SEQ ID | TCTCGCCTTGATATTGGCTTAATTTCAGG |
| NO: 101) | TTGAAAGTAAGAACGGGAC (SEQ ID NO: 184) |
| | TGTCTTGATCCCGTCGGTGGAGAAACGTAGT |
| | GTGACACTCAACCAGCAGA |
| | (SEQ ID NO: 185) |
| 3300012971 | TCTACACCTATGACTGTTGGCCGCAAG |
| (SEQ ID | AAAAGCCGCTCAAGGCGCTA |
| NO: 102) | TGGGACACGGCGCATGA (SEQ ID NO: 186) |
| | GGTCTACACCTATGACTGTTGGCCGCA AGAAAAGCCGCTCAAGGCGC |
| | TATGGGACACGGCGCATG |
| | (SEQ ID NO: 187) |
| SRR6266511 | ACTTGCGAGTGATGCACGCAAGTGAAG |
| (SEQ ID | TGCGGCTTGAGATTGTTGAAAAGCATT |
| NO: 123) | GACACTCCCACCGCTGGAAGCGGCGGG |
| | ATTCTTGCTTCAACG (SEQ ID NO: 188) |
| | CTACAGATGCATGCTGCGGGAGAAACC |
| | GCCCCAAGCGCTGTGGGACA |
| | CAGCGCGTGAGATGGACGTGACCACAA |
| | GCCTAT (SEQ ID NO: 189) |
| SRR6837571 | TAGTTCACCCGTTGTTGGATGTGCTTA |
| (SEQ ID | ACTTTTACAACTTTGGTG (SEQ |
| NO: 145) | ID NO: 190) |
| | GTTTGTGAAAACGGAAGGGCTGTAATC |
| | TCGCCTTGATATTGGCTTAAT |
| | TTCAGGTTGAAAGTAAG (SEQ ID NO: 191) |

CLUST.342201

The spacer length of RNA guides can range from about 12 to 62 nucleotides. In some embodiments, the spacer length of RNA guides can range from about 19 to 40 nucleotides. In some embodiments, the spacer length of an RNA guide is at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides, from 15 to 23 nucleotides, from 16 to 22 nucleotides, from 17 to 20 nucleotides, from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 40, or 45 nucleotides), from 30 or 35 to 40 nucleotides, from 41 to 45 nucleotides, from 45 to 50 nucleotides, from 50 to 62 nucleotides, or longer.

In some embodiments, the direct repeat length of the RNA guide is at least 16 nucleotides, or is from 16 to 20 nucleotides (e.g., 16, 17, 18, 19, or 20 nucleotides). In some embodiments, the direct repeat length of the RNA guide is 19 nucleotides. In some embodiments, the direct repeat length of the RNA guide is greater than 20 nucleotides. See TABLE 18.

Exemplary full-length direct repeat sequences (e.g., direct repeat sequences of pre-crRNAs or unprocessed crRNAs) are shown in Table 14. See also TABLE 18.

TABLE 14
Exemplary direct repeat sequences of
pre-crRNA sequences.

| | pre-crRNA Direct |
|---|---|
| Effector | Repeat Sequence |
| 3300006417 | GTTCACCCCACGGGTGCGT |
| (SEQ ID NO: 301) | GGAGTGATGG (SEQ ID |
| | NO: 342) |
| 3300014060 | CCCTCACTCCACACCCGCG |
| (SEQ ID NO: 306) | CGGGGTAACC (SEQ ID |
| | NO: 384) |
| 3300020180 | CAGCGCACGAGGAGGTGCTG |
| (SEQ ID NO: 307) | (SEQ ID NO: 346) |
| 3300013127 | CCAATCGCCCACGTACGCGT |
| (SEQ ID NO: 316) | GGGCCCGAC (SEQ ID |
| | NO: 390) |
| 3300007960 | CCCTCACCCCACACGCACGT |
| (SEQ ID NO: 317) | GGGGCGTAT (SEQ ID |
| | NO: 391) |
| 3300025687 | CCCTCACCCCACACGCACGT |
| (SEQ ID NO: 319) | GGGGCGTAT (SEQ ID |
| | NO: 391) |
| 3300017963 | CGTGTGAATGCGTGAAATTG |
| (SEQ ID NO: 320) | AGTAGAGGATATGTCAG |
| | (SEQ ID NO: 352) |
| 3300017971 | GTGTGAATGCGTGAAATTGA |
| (SEQ ID NO: 323) | GTAGAGGATATGTCAG |
| | (SEQ ID NO: 353) |
| OVSM01005865 | TATGTAATTATGTATGTAGT |
| (SEQ ID NO: | TTTATGTGTAATATT |
| 332) | (SEQ ID NO: 397) |
| 3300009089 | ACCTCGTGCTCACTCAACTC |
| (SEQ ID NO: 337) | GATGATCTGCTCAA (SEQ |
| | ID NO: 400) |
| 3300006417 | GTTCACCCCACAGGCGCGTG |
| (SEQ ID NO: 340) | GAGTGATGG (SEQ ID |
| | NO: 362) |

In some embodiments, PAMs corresponding to effectors of the present application are set forth as 5′-AAG-3′, 5′-AAD-3′, 5′-AAR-3′, 5′-RAAG-3′ (SEQ ID NO: 921), 5′-RAAR-3′ (SEQ ID NO: 922), 5′-RAAD-3′ (SEQ ID NO: 923). As used herein, R corresponds to A or G, and D corresponds to A or G or T.

In some embodiments, an RNA guide further comprises a tracrRNA. In some embodiments, the tracrRNA is a portion of the non-coding sequences shown in TABLE 19. For example, in some embodiments, the optional tracrRNA is a sequence of TABLE 15.

TABLE 15
Exemplary tracrRNA sequences.

| Effector | tracrRNA Sequence |
|---|---|
| 3300006417 | ACTACATGGCGAAACCACGAACCAAACAAGAGTACGGCCCGTTTAGCG |
| (SEQ ID NO: | TGCGTCTGCCTGTTG (SEQ ID NO: 403) |
| 301) | AAGAGATGCGGTCGCTTCACTCGATGGTGCTTGTTCTACTACGCTCCGCT |
| | TTAGAAGCGCGTCTTCAAGCAGAATCAACATCATCGAGTGTTGGGAA |
| | (SEQ ID NO: 404) |
| | GAACAAGAGATGCGGTCGCTTCACTCGATGGTGCTTGTTCTACTACGCT |
| | CCGCTTTAGAAGCGCGTCTTCAAGCAGAATCAACATCATCGAGTGTTGG |
| | GAACGGCG (SEQ ID NO: 405) |
| | AGTGGTTTGACTGTTGCGTTCATGGTGTATTTATTTTACCT (SEQ ID NO: |
| | 453) |
| | GTTCGTGGTTTCGCCATGTAGTTATTCTACCACCAAAACAGGTACACGG |
| | TCAAC (SEQ ID NO: 406) |
| 3300014060 | GGTGATATGGCAAAGAAAGAGAGACTCAAGACCATCAACATCTTGTTG |
| (SEQ ID NO: | CGCTTGCCGCCCGAGCTTCACGCCGCGCTGAAAAGGTGGGCGCAAGAG |
| 306) | GA (SEQ ID NO: 407) |
| | TGATATGGCAAAGAAAGAGAGACTCAAGACCATCAACATCTTGTTGCGC |
| | TTGCCGCCCGAGCTTCACGCCGCGCTGAAAAGGTGGGCGCAAGAGGA |
| | (SEQ ID NO: 408) |
| | TGGTGATATGGCAAAGAAAGAGAGACTCAAGACCATCAACATCTTGTTG |
| | C (SEQ ID NO: 409) |
| | GGCAAAGAAAGAGAGACTCAAGACCATCAACATCTTGTTGCGCTTGCCG |
| | CCCGAGCTTCACGCCGCGCTGAAAAGGTGGGCGCAAGAGGAACGTCGC |
| | CCGCTAAACTGGCAGATCGTGCGCGTTCT (SEQ ID NO: 410) |
| | GGTGATATGGCAAAGAAAGAGAGACTCAAGACCATCAACATCTTGTTG |
| | CGCTTGCCGCCCGAGCTTCACGCCGCGCTGAAAAGGTGGGCGCAAGAG |
| | GAACGTCGCCCGCTAAACTGGCAGATCGTGCGCGT (SEQ ID NO: 411) |
| 3300020180 | CTGCGAAGAACATTGTCGCGGCCGGGCAGGTCGAGACGAAAAACGGAC |
| (SEQ ID NO: | GTGGAGGGCATGTGAGACGGATGGTGCCTTTGGGCACCTGCGCAGTGCT |
| 307) | CGATGAAGCGT (SEQ ID NO: 412) |
| | TGCGAAGAACATTGTCGCGGCCGGGCAGGTCGAGACGAAAAACGGACG |
| | TGGAGGGCATGTGAGACGGATGGTGCCTTTGGGCACCTGCGCAGTGCTC |
| | GATG (SEQ ID NO: 413) |
| | CGCACGAGGAGGTGCTGTGGAGCCCAGGAAGAGAGGCACCAGCGCACG |
| | AGGAGGTGCTGTGGAGCCCAGGAAGAGAGGCACCAGCGCACGAGGAG |
| | GTGCTGGTAGCCATCGGGTAGGTC (SEQ ID NO: 414) |
| | AGCGCACGAGGAGGTGCTGTGGAGCCCAGGAAGAGAGGCACCAGCGCA |
| | CGAGGAGGTGCTGTGGAGCCCAGGAAGAGAGGCACCAGCGCACGAGGA |
| | GGTGCTGGTAGCCATCGGGTAGGTCGAAAATAT (SEQ ID NO: 415) |
| | TCGAGACGAAAAACGGACGTGGAGGGCATGTGAGACGGATGGTGCCTT |
| | TGGGCACCTGCGCAGTGCTCGATGAAGCGTCAA (SEQ ID NO: 416) |
| 3300013127 | CCTCAACATCCGGCGCGGGCGGCTACAGACTGCGGGTTGTCAGATTGTT |
| (SEQ ID NO: | AAATGCCGCCCGCA (SEQ ID NO: 417) |
| 316) | CCTCAACATCCGGCGCGGGCGGCTACAGACTGCGGGTTGTCAGATTGTT |
| | AAATGCCGCCCGCACC (SEQ ID NO: 418) |
| | TGAACATTGCGTCGAATCTCGCTACCGTCAAATCGGCGGAAAGTCAAGC |
| | CTGTGGATAGGCCGGC (SEQ ID NO: 419) |
| | CCCGCAGTCTGTAGCCGCCCGCGCCGGATGTTGAGGAGTAAACAATGGA |
| | CACTCT (SEQ ID NO: 420) |
| 3300007960 | TGACCTCAGTACGAAGTGCACGGCAGGCGCACCGAGTACCGGACGGCT |
| (SEQ ID NO: | TGAGACATG (SEQ ID NO: 421) |
| 317) | TGCGCCTGCCGTGCACTTCGTACTGAGGTCAAGTATACTGCAAAATTCT |
| | GCACTTGTCAAGCATTAATTTGCGTGAGTTTCTTGACAGTCTGCACAAGT |
| | TTGCAGTATAATGGACTCATGAACAGAGCACACC (SEQ ID NO: 422) |
| | TTTAGCTACTTAAAAACAGAGTTCAATCGTTTGCGCAGTGAAGACGGGT |
| | TTGCCCCGT (SEQ ID NO: 423) |
| | CGGGTTTGCCCCGTTTGTATCAGAGGTTCAAAGCTACGCATATCAGCAG |
| | GCGTCAAAGACTTGTAACGCTTGCGGGGAAAAGGTAGATGCATTGCC |
| | (SEQ ID NO: 424) |
| | CATTTCACCAACAATCCCGCTGGCAGGCGGAGAGAGGCAGGCAGAACA |
| | TGAACACACGCCC (SEQ ID NO: 425) |
| 3300025687 | TAGCTACTTAAAAACAGAGTTCAATCGTTTGCGCAGTGAAGACGGGTTT |
| (SEQ ID NO: | GCCCCGTTTGTATCAGAG (SEQ ID NO: 426) |
| 319) | GGTGTCTCGGTTTACGTGGAACTGGGCATTAGCAGCGTATAACGATGGC |
| | GTTCCGTTTAGCTACTTAAAAACAGAGTTCAATCGTTTGCGCAGTGAAG |
| | ACGGGTTTGCCCCGT (SEQ ID NO: 427) |
| | GTCTCGGTTTACGTGGAACTGGGCATTAGCAGCGTATAACGATGGCGTT |
| | CCGTTTAGCTACTTAAAAACAGAGTTCAATCGTTTGCGCAGTGAAGACG |
| | GGTTTGCCCCGTTTGT (SEQ ID NO: 428) |
| | CTACTTAAAAACAGAGTTCAATCGTTTGCGCAGTGAAGACGGGTTTGCC |
| | CCGTTTGTATC (SEQ ID NO: 429) |
| | GACGCATTTGTTGGTTCGCCGGAATGGGACGAACACGACATTCGGGAAC |
| | GCATGGAGGAAGAAGCAGAGT (SEQ ID NO: 430) |
| 3300017971 | AACAGTATTGAATGTCGCCGTCGGGCAGACGGAGACTCTAAACGCTCGC |
| (SEQ ID NO: | TGAGCGGACGGCCGCTGTCGTGGAAGGATGTGC (SEQ ID NO: 431) |
| 323) | AGTATTGAATGTCGCCGTCGGGCAGACGGAGACTCTAAACGCTCGCTGA |
| | GCGGACGGCCGCTGTCGTGGAAGGATGTGCTCTTTCGCGTTCATGCTGA |
| | CTGCGTTGAAGCGAGAATCCCTCTACG (SEQ ID NO: 432) |
| | TCTTTAACAGTATTGAATGTCGCCGTCGGGCAGACGGAGACTCTAAACG |
| | CTCGCTGAGCGGACGGCCGCTGTCGTGGAAGGATGTGCTCTTTCGCGTT |
| | CATGCTGACTGCGTTGAAGCGAGAATCCCTCTACG (SEQ ID NO: 433) |
| | TCTTTAACAGTATTGAATGTCGCCGTCGGGCAGACGGAGACTCTAAACG |
| | CTCGCTGAGCGGACGGCCGCTGTCGTGGAAGGATGTGCTCTTTCGCGTT |
| | CATGCTGACTGCGTTGAAGCGAGAATCCCTCTACGATTCG (SEQ ID NO: |
| | 434) |
| OVSM0100586 | GAATCTGTTAGTAGATTTTCATTATTAGAAGCACACTTAACTCCCTTGTT |
| 5 (SEQ ID NO: | GGTGTGCTTCTCTCTTTTCTAC (SEQ ID NO: 435) |
| 332) | CAGTCTTTAGTTTCTCGTTTGAGGGAGTTGGATAATACGTATTGGGATTG |
| | TTTTTTAAAGAGAAAAGAAGGAGCCCAACTTCCTTGTGGCGTTA (SEQ ID |
| | NO: 436) |
| | TTAGTTTCTCGTTTGAGGGAGTTGGATAATACGTATTGGGATTGTTTTTT |
| | AAAGAGAAAAGAAGGAGCCCAACTTCCTTGTGGCGTTAT (SEQ ID NO: |
| | 437) |
| | CAATGATCATTGCAAAAGGGATGCGAAAATAGGTTTTTAAGTGATAATC |
| | CACAAAACATATCAGTTTAGATTAAAGCCAACAAAAGAACAGGAGGCT |
| | TTGTTTTGGCGGTTTTC (SEQ ID NO: 438) |
| 3300009089 | GAGCAGGAGCTACTGCGAGGAGCAGACTGTGACGAAGCGAGCATACAA |
| (SEQ ID NO: | GTTCAGGTGTTATCCGACAGATGAGCAAAAGCAGATTCTTG (SEQ ID NO: |
| 337) | 439) |
| | GAGCAGGAGCTACTGCGAGGAGCAGACTGTGACGAAGCGAGCATACAA |
| | GTTCAGGTGTT (SEQ ID NO: 440) |
| | GAAAGAGAGAATCCTCGGACGAGCCGCGCAGCTTTTCAACCAGCAGGG |
| | ATACTTCGGCTCGTCACTCTCCGATAT (SEQ ID NO: 441) |
| | CATGTAGGCATGTCCTAGATGTCTAAGTATTGGAGAACGGATTTTTATG |
| | AGCAAAGGTGAGCAAACG (SEQ ID NO: 442) |
| | ACGAAGCGAGCATACAAGTTCAGGTGTTATCCGACAGATGAGCAAAAG |
| | CAGATTCTTGCTCGCACGTTCGGCTGCTGTCGCTGGGTCTACAA (SEQ ID |
| | NO: 443) |
| | ACGAAGCGAGCATACAAGTTCAGGTGTTATCCGACAGATGAGCAAAAG |
| | CAGATTCTTGCTCGCACGTTCGGCTGCTGTCGCTGGGTCT (SEQ ID |
| | NO: 444) |
| 3300006417 | GCGGTAGTAATCACGTCGAGACGCAAAACGCCTGGGGACGGTGTAGGT |
| (SEQ ID NO: | AGCAAACCGGATGAACCAGGAGTTCACGCAATTGAATAGGT (SEQ ID |
| 340) | NO: 445) |
| | GTCGCGGTAGTAATCACGTCGAGACGCAAAACGCCTGGGGACGGTGTA |
| | GGTAGCAAACCGGATGAACCAGGAGTTCACGCAATTGAATAG (SEQ ID |
| | NO: 446) |
| | GAGGCCGTGAAGATGGCAGGCGCTGCCTAGTGCGCCCGTCGCGGTAGT |
| | AATCACGTCGAGACGCAAAACGCCTGGGGACGGTGTAGGTAGCAAACC |
| | GGATGAACCAGGAGTTCACGCAATTGAATAGGTG (SEQ ID NO: 447) |
| | CGCAGCGGTCAACATCCGCAACGAGGCCGTGAAGATGGCAGGCGCTGC |
| | CTAGTGCGCCCGTCGCGGTAGTAATCACGTCGAGACGCAAAACGCCTGG |
| | GGACGGTGTAGGTAGCAAACCGGATGAACCAGGAGTTCACGCAA (SEQ |
| | ID NO: 448) |
| | CTATCGGTGCGCTGGTGGCAGTGCCCGACCTGCGGCGCAGAACATGATC |
| | GGGACGGCAACGCA (SEQ ID NO: 449) |
| | GCTGGCTGAGTTGAACGCCGCCTATGACCGGGGTGAACGGCCTGCGATT |
| | GGAACCTGCAATGAGTGCG (SEQ ID NO: 450) |

CLUST.1950099

The spacer length of RNA guides can range from about 15 to 55 nucleotides. The spacer length of RNA guides can range from about 20 to 39 nucleotides. In some embodiments, the spacer length of an RNA guide is at least 15 nucleotides, at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides, from 15 to 23 nucleotides, from 16 to 22 nucleotides, from 17 to 20 nucleotides, from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 40, or 45 nucleotides), from 30 or 35 to 40 nucleotides, from 41 to 45 nucleotides, from 45 to 50 nucleotides, from 50 to 55 nucleotides, or longer.

In some embodiments, the direct repeat length of the RNA guide is at least 16 nucleotides, or is from 16 to 20 nucleotides (e.g., 16, 17, 18, 19, or 20 nucleotides). In some embodiments, the direct repeat length of the RNA guide is about 39 nucleotides. See TABLE 24.

Exemplary full-length direct repeat sequences (e.g., direct repeat sequences of pre-crRNAs or unprocessed crRNAs) is shown in Table 20. See also TABLE 24.

TABLE 20
Exemplary direct repeat sequences
of pre-crRNA sequences.

| Effector | pre-crRNA Direct Repeat Sequence |
|---|---|
| SRR6201554 | CCAGCAACAGCCGCGTGGGGCT |
| (SEQ ID NO: 501) | ACTAGTACTGCGAC |
| | (SEQ ID NO: 522) |
| 3300028603 (SEQ | CCAACAACAGCCGCGCGGGGCT |
| ID NO: 517) | GCGAATACTGCGAC |
| | (SEQ ID NO: 529) |

In some embodiments, the mature crRNA (e.g., direct repeat sequences of processed crRNAs) corresponding to the effector of SEQ ID NO: 501 is CAACAGCCGCGTGGGGCTACTAGTACTGCG (SEQ ID NO: 535).

In some embodiments, PAMs corresponding to effectors of the present application are set forth as 5′-TTN-3′. As used herein, N's can each be any nucleotide (e.g., A, G, T, or C) or a subset thereof (e.g., R (A or G), Y (C or T), K (G or T), B (G, T, or C), H (A, C, or T).

In some embodiments, an RNA guide further comprises a tracrRNA. In some embodiments, the tracrRNA is not required (e.g., the tracrRNA is optional). In some embodiments, the tracrRNA is a portion of the non-coding sequences shown in TABLE 25. For example, in some embodiments, the optional tracrRNA is a sequence of TABLE 21.

TABLE 21
Exemplary tracrRNA sequences.

| Effector | tracrRNA Sequence |
|---|---|
| SRR6201554 | GAATTGGTGACGGTGTGTCCGGCT |
| (SEQ ID NO: 501) | TGCGGCACCGCCAGGGACAAGGA |
| | CGTTGGCGCGGCCGAGGTGA |
| | (SEQ ID NO: 536) |
| | GACACCGCACCAACAGCCGCGTGG |
| | GGCTTCCAATACCTCGACAGTCG |
| | CCCCACCGCTCGACGTGCGAACCC |
| | CA (SEQ ID NO: 537) |
| | CACCGCCAGGGACAAGGACGTTGG |
| | CGCGGCCGAGGTGATCCTTCGGC |
| | GCGCAGAGGAGGCGCTCGCAAAGC |
| | ATTCTGCAGAGTAGCGAAATCAA |
| | (SEQ ID NO: 538) |

CLUST.057059

The spacer length of RNA guides can range from about 15 to 50 nucleotides. In some embodiments, the spacer length of RNA guides can range from about 20 to 44 nucleotides. In some embodiments, the spacer length of an RNA guide is at least at least 15 nucleotides, at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides, from 15 to 23 nucleotides, from 16 to 22 nucleotides, from 17 to 20 nucleotides, from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 40, or 45 nucleotides), from 30 or 35 to 40 nucleotides, from 41 to 45 nucleotides, from 45 to 50 nucleotides, or longer.

In some embodiments, the direct repeat length of the RNA guide is at least 16 nucleotides, or is from 16 to 20 nucleotides (e.g., 16, 17, 18, 19, or 20 nucleotides). In some embodiments, the direct repeat length of the RNA guide is 19 nucleotides. In some embodiments, the direct repeat length of the RNA guide is greater than 20 nucleotides. See TABLE 30.

Exemplary full-length direct repeat sequences (e.g., direct repeat sequences of pre-crRNAs or unprocessed crRNAs) are shown in Table 26. See also TABLE 30.

TABLE 26
Exemplary direct repeat sequences
of pre-crRNA sequences.

| | |
|---|---|
| Effector | pre-crRNA Direct Repeat Sequence |
| 3300023179 (SEQ | CTTGCAACTGGGCTTGGGGACTG |
| ID NO: 601) | AGGATAGTTGAAAC (SEQ ID NO: 683) |
| 3300002382 (SEQ | CTTGCAACTAAGACTGGGGACTG |
| ID NO: 615) | AGGATAGTTGAAACT (SEQ ID NO: 693) |

In some embodiments, PAMs corresponding to effectors of the present application are set forth as 5′-GTN-3′. As used herein, N's can each be any nucleotide (e.g., A, G, T, or C) or a subset thereof (e.g., R (A or G), Y (C or T), K (G or T), B (G, T, or C), H (A, C, or T).

In some embodiments, an RNA guide further comprises a tracrRNA. In some embodiments, the tracrRNA is not required (e.g., the tracrRNA is optional). In some embodiments, the tracrRNA is a portion of the non-coding sequences shown in TABLE 31. For example, in some embodiments, the optional tracrRNA is a sequence of TABLE 27.

TABLE 27
Exemplary tracrRNA sequences.

| Effector | tracrRNA Sequence |
|---|---|
| 3300023179 (SEQ | GACAGTAAACAAAAAATAAGATGGAC |
| ID NO: 601) | TATATTTATAAACCAGGAAACATAGT |
| | TATTACAATTGCATTGTAGGTTTCAA |
| | CCTGATACCCACTAAAGCGTGTTAGT |
| | GGGTATTTTTTATTTA |
| | (SEQ ID NO: 739) |
| | AAACAAAAAATAAGATGGACTATATT |
| | TATAAACCAGGAAACATAGTTATTAC |
| | AATTGCATTGTAGGTTTCAACCTGAT |
| | ACCCACTAAAGCGTGTTAGTGGGTAT |
| | TTTTTATTTATG |
| | (SEQ ID NO: 740) |
| | AATATTAAATTACCGCAGTTTGGGC |
| | GGGTTGCTAATAATATTAGCTTA |
| | TGACTTCTAAGGCTTTTGCCTAAAA |
| | AGTAAGGGGGAAGGCGACAACC |
| | CCCAAAGACCAGCCGGAACTATGGC |
| | TGGCAACTATCTCATCTTTTTGG |
| | CATATCAAAGCTAGGGCAAAAACCC |
| | CAGACATTCGCCAAAAGCCCAG |
| | AACCATGACATTGCAAGAGTTTCGC |
| | CCAGTTTCTTTTAAAGACCCAAG |
| | CTGATTTAAGCGGCTGAAATGAGAT |
| | TTTTTA (SEQ ID NO: 741) |
| | ATATTAAATTACCGCAGTTTGGGC |
| | GGGTTGCTAATAATATTAGCTTAT |
| | GACTTCTAAGGCTTTTGCCTAA |
| | (SEQ ID NO: 742) |
| | TTATCCAAACTAGCGACTAAGTAC |
| | CTGGTAATAAAAACGTGATTACT |
| | TTGA (SEQ ID NO: 743) |
| | CCAAACTAGCGACTAAGTACCTG |
| | GTAATAAAAACGTGATTACTTTGA |
| | A (SEQ ID NO: 744) |
| 3300002382 (SEQ | AAATTACCGCAGTTTGGGCGGGCA |
| ID NO: 615) | GCTAATAATATTAGCTTATGACTT |
| | CTAAGGCTTTTGCCTAAAAAGT |
| | (SEQ ID NO: 745) |
| | TTAAATTACCGCAGTTTGGGCGGG |
| | CAGCTAATAATATTAGCTTATGAC |
| | TTCTAAGGCTTTTGCCTAAAAA |
| | (SEQ ID NO: 746) |
| | CTGAAATGAGATTTTTTAATGTGT |
| | AATCGTCTAGCCTGGGAACATATA |
| | TGCAACTTGCAATTAACTCAGTTC |
| | CTCAAGTCCGGTTTCACCTA (SEQ |
| | ID NO: 747) |
| | ATGAGATTTTTTAATGTGTAATCG |
| | TCTAGCCTGGGAACATATATGCAA |
| | CTTGCAATTAACTCAGTTCCTCAA |
| | GTCCGGTTTCACCTATTA (SEQ ID |
| | NO: 748) |
| | CCCAGACATTCGCCAAAAGCCCAG |
| | AACCATGACATTGCAAGAGTTTC |
| | GCCCAGTTTCTTTTAAAGATTCAA |
| | GCTGATTTAAGCGGCTGAAATGAG |
| | ATTTTTTAATGTGTAATCGT |
| | (SEQ ID NO: 749) |
| | CCCCAGACATTCGCCAAAAGCCCA |
| | GAACCATGACATTGCAAGAGTTT |
| | CGCCCAGTTTCTTTTAAAGATTCAA |
| | GCTGATTTAAGCGGCTGAAATGA |
| | GATTTTTTAATGT (SEQ ID NO: 750) |

The RNA guide sequences can be modified in a manner that allows for formation of the CRISPR complex and successful binding to the target, while at the same time not allowing for successful nuclease activity (i.e., without nuclease activity/without causing indels). These modified guide sequences are referred to as “dead guides” or “dead guide sequences.” These dead guides or dead guide sequences may be catalytically inactive or conformationally inactive with regard to nuclease activity. Dead guide sequences are typically shorter than respective guide sequences that result in active cleavage. In some embodiments, dead guides are 5%, 10%, 20%, 30%, 40%, or 50% shorter than respective RNA guides that have nuclease activity. Dead guide sequences of RNA guides can be from 13 to 15 nucleotides in length (e.g., 13, 14, or 15 nucleotides in length), from 15 to 19 nucleotides in length, or from 17 to 18 nucleotides in length (e.g., 17 nucleotides in length).

Thus, in one aspect, the disclosure provides non-naturally occurring or engineered CRISPR systems including functional CLUST.133120, CLUST.099129, CLUST.342201, CLUST.195009, and CLUST.057059 CRISPR effectors as described herein, and an RNA guide wherein the RNA guide comprises a dead guide sequence, whereby the RNA guide is capable of hybridizing to a target sequence such that the CRISPR system is directed to a genomic locus of interest in a cell without detectable cleavage activity. A detailed description of dead guides is described, e.g., in WO 2016094872, which is incorporated herein by reference in its entirety.

Inducible RNA Guides

RNA guides can be generated as components of inducible systems. The inducible nature of the systems allows for spatiotemporal control of gene editing or gene expression. In some embodiments, the stimuli for the inducible systems include, e.g., electromagnetic radiation, sound energy, chemical energy, and/or thermal energy.

In some embodiments, the transcription of RNA guide can be modulated by inducible promoters, e.g., tetracycline or doxycycline controlled transcriptional activation (Tet-On and Tet-Off expression systems), hormone inducible gene expression systems (e.g., ecdysone inducible gene expression systems), and arabinose-inducible gene expression systems. Other examples of inducible systems include, e.g., small molecule two-hybrid transcription activations systems (FKBP, ABA, etc.), light inducible systems (Phytochrome, LOV domains, or cryptochrome), or Light Inducible Transcriptional Effector (LITE). These inducible systems are described, e.g., in WO 2016205764 and U.S. Pat. No. 8,795,965, each of which is incorporated herein by reference in its entirety.

Chemical Modifications

Chemical modifications can be applied to the phosphate backbone, sugar, and/or base of the RNA guide. Backbone modifications such as phosphorothioates modify the charge on the phosphate backbone and aid in the delivery and nuclease resistance of the oligonucleotide (see, e.g., Eckstein, “Phosphorothioates, essential components of therapeutic oligonucleotides,” Nucl. Acid Ther., 24 (2014), pp. 374-387); modifications of sugars, such as 2′-O-methyl (2′-OMe), 2′-F, and locked nucleic acid (LNA), enhance both base pairing and nuclease resistance (see, e.g., Allerson et al. “Fully 2′-modified oligonucleotide duplexes with improved in vitro potency and stability compared to unmodified small interfering RNA,” J. Med. Chem., 48.4 (2005): 901-904). Chemically modified bases such as 2-thiouridine or N6-methyladenosine, among others, can allow for either stronger or weaker base pairing (see, e.g., Bramsen et al., “Development of therapeutic-grade small interfering RNAs by chemical engineering,” Front. Genet., 2012 Aug. 20; 3:154). Additionally, RNA is amenable to both 5′ and 3′ end conjugations with a variety of functional moieties including fluorescent dyes, polyethylene glycol, or proteins.

A wide variety of modifications can be applied to chemically synthesized RNA guide molecules. For example, modifying an oligonucleotide with a 2′-OMe to improve nuclease resistance can change the binding energy of Watson-Crick base pairing. Furthermore, a 2′-OMe modification can affect how the oligonucleotide interacts with transfection reagents, proteins or any other molecules in the cell. The effects of these modifications can be determined by empirical testing.

In some embodiments, the RNA guide includes one or more phosphorothioate modifications. In some embodiments, the RNA guide includes one or more locked nucleic acids for the purpose of enhancing base pairing and/or increasing nuclease resistance.

A summary of these chemical modifications can be found, e.g., in Kelley et al., “Versatility of chemically synthesized guide RNAs for CRISPR-Cas9 genome editing,” J. Biotechnol. 2016 Sep. 10; 233:74-83; WO 2016205764; and U.S. Pat. No. 8,795,965, each which is incorporated by reference in its entirety.

Sequence Modifications

The sequences and the lengths of the RNA guides, tracrRNAs, and crRNAs described herein can be optimized. In some embodiments, the optimized length of RNA guide can be determined by identifying the processed form of tracrRNA and/or crRNA, or by empirical length studies for RNA guides, tracrRNAs, crRNAs, and the tracrRNA tetraloops.

The RNA guides can also include one or more aptamer sequences. Aptamers are oligonucleotide or peptide molecules that can bind to a specific target molecule. The aptamers can be specific to gene effectors, gene activators, or gene repressors. In some embodiments, the aptamers can be specific to a protein, which in turn is specific to and recruits/binds to specific gene effectors, gene activators, or gene repressors. The effectors, activators, or repressors can be present in the form of fusion proteins. In some embodiments, the RNA guide has two or more aptamer sequences that are specific to the same adaptor proteins. In some embodiments, the two or more aptamer sequences are specific to different adaptor proteins. The adaptor proteins can include, e.g., MS2, PP7, Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7 s, and PRR1. Accordingly, in some embodiments, the aptamer is selected from binding proteins specifically binding any one of the adaptor proteins as described herein. In some embodiments, the aptamer sequence is a MS2 loop. A detailed description of aptamers can be found, e.g., in Nowak et al., “Guide RNA engineering for versatile Cas9 functionality,” Nucl. Acid. Res., 2016 Nov. 16;44(20):9555-9564; and WO 2016205764, each of which is incorporated herein by reference in its entirety.

Guide: Target Sequence Matching Requirements

In CRISPR systems, the degree of complementarity between a guide sequence and its corresponding target sequence can be about 50%, 60%, 75%, 80%, 85%, 90%, 95%, 97.5%, 99%, or 100%. To reduce off-target interactions, e.g., to reduce the guide interacting with a target sequence having low complementarity, mutations can be introduced to the CRISPR systems so that the CRISPR systems can distinguish between target and off-target sequences that have greater than 80%, 85%, 90%, or 95% complementarity. In some embodiments, the degree of complementarity is from 80% to 95%, e.g., about 83%, 84%, 85%, 86%, 87%, 88%, 89%, 90%, 91%, 92%, 93%, 94%, or 95% (for example, distinguishing between a target having 18 nucleotides from an off-target of 18 nucleotides having 1, 2, or 3 mismatches). Accordingly, in some embodiments, the degree of complementarity between a guide sequence and its corresponding target sequence is greater than 94.5%, 95%, 95.5%, 96%, 96.5%, 97%, 97.5%, 98%, 98.5%, 99%, 99.5%, or 99.9%. In some embodiments, the degree of complementarity is 100%.

It is known in the field that complete complementarity is not required provided that there is sufficient complementarity to be functional. Modulations of cleavage efficiency can be exploited by introduction of mismatches, e.g., one or more mismatches, such as 1 or 2 mismatches between spacer sequence and target sequence, including the position of the mismatch along the spacer/target. The more central (i.e., not at the 3′ or 5′ ends) a mismatch, e.g., a double mismatch, is located; the more cleavage efficiency is affected. Accordingly, by choosing mismatch positions along the spacer sequence, cleavage efficiency can be modulated. For example, if less than 100% cleavage of targets is desired (e.g., in a cell population), 1 or 2 mismatches between spacer and target sequence can be introduced in the spacer sequences.

### Methods of Using CRISPR Systems

The CRISPR systems described herein have a wide variety of utilities including modifying (e.g., deleting, inserting, translocating, inactivating, or activating) a target polynucleotide in a multiplicity of cell types. The CRISPR systems have a broad spectrum of applications in, e.g., DNA/RNA detection (e.g., specific high sensitivity enzymatic reporter unlocking (SHERLOCK)), tracking and labeling of nucleic acids, enrichment assays (extracting desired sequence from background), detecting circulating tumor DNA, preparing next generation library, drug screening, disease diagnosis and prognosis, and treating various genetic disorders.

DNA/RNA Detection

In one aspect, the CRISPR systems described herein can be used in DNA/RNA detection. Single effector RNA-guided DNases can be reprogrammed with CRISPR RNAs (crRNAs) to provide a platform for specific single-stranded DNA (ssDNA) sensing. Upon recognition of its DNA target, activated Type V single effector DNA-guided DNases engage in “collateral” cleavage of nearby non-targeted ssDNAs. This crRNA-programmed collateral cleavage activity allows the CRISPR systems to detect the presence of a specific DNA by nonspecific degradation of labeled ssDNA.

The collateral ssDNA activity can be combined with a reporter in DNA detection applications such as a method called the DNA Endonuclease-Targeted CRISPR trans reporter (DETECTR) method, which achieves attomolar sensitivity for DNA detection (see, e.g., Chen et al., Science, 360(6387):436-439, 2018), which is incorporated herein by reference in its entirety. One application of using the enzymes described herein is to degrade non-specific ssDNA in an in vitro environment. A “reporter” ssDNA molecule linking a fluorophore and a quencher can also be added to the in vitro system, along with an unknown sample of DNA (either single-stranded or double-stranded). Upon recognizing the target sequence in the unknown piece of DNA, the effector complex cleaves the reporter ssDNA resulting in a fluorescent readout.

In other embodiments, the SHERLOCK method (Specific High Sensitivity Enzymatic Reporter UnLOCKing) also provides an in vitro nucleic acid detection platform with attomolar (or single-molecule) sensitivity based on nucleic acid amplification and collateral cleavage of a reporter ssDNA, allowing for real-time detection of the target. Methods of using CRISPR in SHERLOCK are described in detail, e.g., in Gootenberg, et al. “Nucleic acid detection with CRISPR-Cas13a/C2c2,” Science, 356(6336):438-442 (2017), which is incorporated herein by reference in its entirety.

In some embodiments, the CRISPR systems described herein can be used in multiplexed error-robust fluorescence in situ hybridization (MERFISH). These methods are described in, e.g., Chen et al., “Spatially resolved, highly multiplexed RNA profiling in single cells,” Science, 2015 Apr. 24; 348(6233):aaa6090, which is incorporated herein by reference in its entirety.

Tracking and Labeling of Nucleic Acids

Cellular processes depend on a network of molecular interactions among proteins, RNAs, and DNAs. Accurate detection of protein-DNA and protein-RNA interactions is key to understanding such processes. In vitro proximity labeling techniques employ an affinity tag combined with, a reporter group, e.g., a photoactivatable group, to label polypeptides and RNAs in the vicinity of a protein or RNA of interest in vitro. After UV irradiation, the photoactivatable groups react with proteins and other molecules that are in close proximity to the tagged molecules, thereby labelling them. Labelled interacting molecules can subsequently be recovered and identified. The RNA targeting effector proteins can for instance be used to target probes to selected RNA sequences. These applications can also be applied in animal models for in vivo imaging of diseases or difficult-to culture cell types. The methods of tracking and labeling of nucleic acids are described, e.g., in U.S. Pat. No. 8,795,965; WO 2016205764; and WO 2017070605, each of which is incorporated herein by reference in its entirety.

High-Throughput Screening

The CRISPR systems described herein can be used for preparing next generation sequencing (NGS) libraries. For example, to create a cost-effective NGS library, the CRISPR systems can be used to disrupt the coding sequence of a target gene, and the CRISPR effector transfected clones can be screened simultaneously by next-generation sequencing (e.g., on the Ion Torrent PGM system). A detailed description regarding how to prepare NGS libraries can be found, e.g., in Bell et al., “A high-throughput screening strategy for detecting CRISPR-Cas9 induced mutations using next-generation sequencing,” BMC Genomics, 15.1 (2014): 1002, which is incorporated herein by reference in its entirety.

Engineered Cells

Microorganisms (e.g., E. coli, yeast, and microalgae) are widely used for synthetic biology. The development of synthetic biology has a wide utility, including various clinical applications. For example, the programmable CRISPR systems can be used to split proteins of toxic domains for targeted cell death, e.g., using cancer-linked RNA as target transcript. Further, pathways involving protein-protein interactions can be influenced in synthetic biological systems with e.g., fusion complexes with the appropriate effectors such as kinases or enzymes.

In some embodiments, RNA guide sequences that target phage sequences can be introduced into the microorganism. Thus, the disclosure also provides methods of “vaccinating” a microorganism (e.g., a production strain) against phage infection.

In some embodiments, the CRISPR systems provided herein can be used to engineer microorganisms, e.g., to improve yield or improve fermentation efficiency. For example, the CRISPR systems described herein can be used to engineer microorganisms, such as yeast, to generate biofuel or biopolymers from fermentable sugars, or to degrade plant-derived lignocellulose derived from agricultural waste as a source of fermentable sugars. More particularly, the methods described herein can be used to modify the expression of endogenous genes required for biofuel production and/or to modify endogenous genes, which may interfere with the biofuel synthesis. These methods of engineering microorganisms are described e.g., in Verwaal et al., “CRISPR/Cpf1 enables fast and simple genome editing of Saccharomyces cerevisiae,” Yeast, 2017 Sep. 8. doi: 10.1002/yea.3278; and Hlavova et al., “Improving microalgae for biotechnology—from genetics to synthetic biology,” Biotechnol. Adv., 2015 Nov. 1; 33:1194-203, each of which is incorporated herein by reference in its entirety.

In some embodiments, the CRISPR systems provided herein can be used to engineer eukaryotic cells or eukaryotic organisms. For example, the CRISPR systems described herein can be used to engineer eukaryotic cells not limited to a plant cell, a fungal cell, a mammalian cell, a reptile cell, an insect cell, an avian cell, a fish cell, a parasite cell, an arthropod cell, an invertebrate cell, a vertebrate cell, a rodent cell, a mouse cell, a rat cell, a primate cell, a non-human primate cell, or a human cell. In some embodiments, eukaryotic cell is in an in vitro culture. In some embodiments, the eukaryotic cell is in vivo. In some embodiments, the eukaryotic cell is ex vivo.

Gene Drives

Gene drive is the phenomenon in which the inheritance of a particular gene or set of genes is favorably biased. The CRISPR systems described herein can be used to build gene drives. For example, the CRISPR systems can be designed to target and disrupt a particular allele of a gene, causing the cell to copy the second allele to fix the sequence. Because of the copying, the first allele will be converted to the second allele, increasing the chance of the second allele being transmitted to the offspring. A detailed method regarding how to use the CRISPR systems described herein to build gene drives is described, e.g., in Hammond et al., “A CRISPR-Cas9 gene drive system targeting female reproduction in the malaria mosquito vector Anopheles gambiae,” Nat. Biotechnol., 2016 January; 34(1):78-83, which is incorporated herein by reference in its entirety.

Pooled-Screening

As described herein, pooled CRISPR screening is a powerful tool for identifying genes involved in biological mechanisms such as cell proliferation, drug resistance, and viral infection. Cells are transduced in bulk with a library of RNA guide-encoding vectors described herein, and the distribution of gRNAs is measured before and after applying a selective challenge. Pooled CRISPR screens work well for mechanisms that affect cell survival and proliferation, and they can be extended to measure the activity of individual genes (e.g., by using engineered reporter cell lines). Arrayed CRISPR screens, in which only one gene is targeted at a time, make it possible to use RNA-seq as the readout. In some embodiments, the CRISPR systems as described herein can be used in single-cell CRISPR screens. A detailed description regarding pooled CRISPR screenings can be found, e.g., in Datlinger et al., “Pooled CRISPR screening with single-cell transcriptome read-out,” Nat. Methods., 2017 March; 14(3):297-301, which is incorporated herein by reference in its entirety.

Saturation Mutagenesis (“Bashing”)

The CRISPR systems described herein can be used for in situ saturating mutagenesis. In some embodiments, a pooled RNA guide library can be used to perform in situ saturating mutagenesis for particular genes or regulatory elements. Such methods can reveal critical minimal features and discrete vulnerabilities of these genes or regulatory elements (e.g., enhancers). These methods are described, e.g., in Canver et al., “BCL11A enhancer dissection by Cas9-mediated in situ saturating mutagenesis,” Nature, 2015 Nov. 12; 527(7577):192-7, which is incorporated herein by reference in its entirety.

Therapeutic Applications

In some embodiments, the CRISPR systems described herein can be used to edit a target nucleic acid to modify the target nucleic acid (e.g., by inserting, deleting, or mutating one or more amino acid residues). For example, in some embodiments the CRISPR systems described herein comprise an exogenous donor template nucleic acid (e.g., a DNA molecule or an RNA molecule), which comprises a desirable nucleic acid sequence. Upon resolution of a cleavage event induced with the CRISPR system described herein, the molecular machinery of the cell can utilize the exogenous donor template nucleic acid in repairing and/or resolving the cleavage event. Alternatively, the molecular machinery of the cell can utilize an endogenous template in repairing and/or resolving the cleavage event. In some embodiments, the CRISPR systems described herein may be used to modify a target nucleic acid resulting in an insertion, a deletion, and/or a point mutation). In some embodiments, the insertion is a scarless insertion (i.e., the insertion of an intended nucleic acid sequence into a target nucleic acid resulting in no additional unintended nucleic acid sequence upon resolution of the cleavage event). Donor template nucleic acids may be double-stranded or single-stranded nucleic acid molecules (e.g., DNA or RNA). Methods of designing exogenous donor template nucleic acids are described, for example, in WO 2016094874, the entire contents of which is expressly incorporated herein by reference.

In another aspect, the disclosure provides the use of a system described herein in a method selected from the group consisting of RNA sequence specific interference; RNA sequence-specific gene regulation; screening of RNA, RNA products, lncRNA, non-coding RNA, nuclear RNA, or mRNA; mutagenesis; inhibition of RNA splicing; fluorescence in situ hybridization; breeding; induction of cell dormancy; induction of cell cycle arrest; reduction of cell growth and/or cell proliferation; induction of cell anergy; induction of cell apoptosis; induction of cell necrosis; induction of cell death; or induction of programmed cell death.

The CRISPR systems described herein can have various therapeutic applications. In some embodiments, the new CRISPR systems can be used to treat various diseases and disorders, e.g., genetic disorders (e.g., monogenetic diseases) or diseases that can be treated by nuclease activity (e.g., Pcsk9 targeting or BCL1 la targeting). In some embodiments, the methods described here are used to treat a subject, e.g., a mammal, such as a human patient. The mammalian subject can also be a domesticated mammal, such as a dog, cat, horse, monkey, rabbit, rat, mouse, cow, goat, or sheep.

The methods can include the condition or disease being infectious, and wherein the infectious agent is selected from the group consisting of human immunodeficiency virus (HIV), herpes simplex virus-1 (HSV1), and herpes simplex virus-2 (HSV2).

In one aspect, the CRISPR systems described herein can be used for treating a disease caused by overexpression of RNAs, toxic RNAs and/or mutated RNAs (e.g., splicing defects or truncations). For example, expression of the toxic RNAs may be associated with the formation of nuclear inclusions and late-onset degenerative changes in brain, heart, or skeletal muscle. In some embodiments, the disorder is myotonic dystrophy. In myotonic dystrophy, the main pathogenic effect of the toxic RNAs is to sequester binding proteins and compromise the regulation of alternative splicing (see, e.g., Osborne et al., “RNA-dominant diseases,” Hum. Mol. Genet., 2009 Apr. 15; 18(8):1471-81). Myotonic dystrophy (dystrophia myotonica (DM)) is of particular interest to geneticists because it produces an extremely wide range of clinical features. The classical form of DM, which is now called DM type 1 (DM1), is caused by an expansion of CTG repeats in the 3′-untranslated region (UTR) of DMPK, a gene encoding a cytosolic protein kinase. The CRISPR systems as described herein can target overexpressed RNA or toxic RNA, e.g., the DMPK gene or any of the mis-regulated alternative splicing in DM1 skeletal muscle, heart, or brain.

The CRISPR systems described herein can also target trans-acting mutations affecting RNA-dependent functions that cause various diseases such as, e.g., Prader Willi syndrome, Spinal muscular atrophy (SMA), and Dyskeratosis congenita. A list of diseases that can be treated using the CRISPR systems described herein is summarized in Cooper et al., “RNA and disease,” Cell, 136.4 (2009): 777-793, and WO 2016205764, each of which is incorporated herein by reference in its entirety.

The CRISPR systems described herein can also be used in the treatment of various tauopathies, including, e.g., primary and secondary tauopathies, such as primary age-related tauopathy (PART)/Neurofibrillary tangle (NFT)-predominant senile dementia (with NFTs similar to those seen in Alzheimer Disease (AD), but without plaques), dementia pugilistica (chronic traumatic encephalopathy), and progressive supranuclear palsy. A useful list of tauopathies and methods of treating these diseases are described, e.g., in WO 2016205764, which is incorporated herein by reference in its entirety.

The CRISPR systems described herein can also be used to target mutations disrupting the cis-acting splicing codes that can cause splicing defects and diseases. These diseases include, e.g., motor neuron degenerative disease that results from deletion of the SMN1 gene (e.g., spinal muscular atrophy), Duchenne Muscular Dystrophy (DMD), frontotemporal dementia, and Parkinsonism linked to chromosome 17 (FTDP-17), and cystic fibrosis.

The CRISPR systems described herein can further be used for antiviral activity, in particular, against RNA viruses. The effector proteins can target the viral RNAs using suitable RNA guides selected to target viral RNA sequences.

Furthermore, in vitro RNA sensing assays can be used to detect specific RNA substrates. The RNA targeting effector proteins can be used for RNA-based sensing in living cells. Examples of applications are diagnostics by sensing of, for examples, disease-specific RNAs.

A detailed description of therapeutic applications of the CRISPR systems described herein can be found, e.g., in U.S. Pat. No. 8,795,965, EP 3009511, WO 2016205764, and WO 2017070605, each of which is incorporated herein by reference in its entirety.

Applications in Plants

The CRISPR systems described herein have a wide variety of utility in plants. In some embodiments, the CRISPR systems can be used to engineer genomes of plants (e.g., improving production, making products with desired post-translational modifications, or introducing genes for producing industrial products). In some embodiments, the CRISPR systems can be used to introduce a desired trait to a plant (e.g., with or without heritable modifications to the genome) or regulate expression of endogenous genes in plant cells or whole plants.

In some embodiments, the CRISPR systems can be used to identify, edit, and/or silence genes encoding specific proteins, e.g., allergenic proteins (e.g., allergenic proteins in peanuts, soybeans, lentils, peas, green beans, and mung beans). A detailed description regarding how to identify, edit, and/or silence genes encoding proteins is described, e.g., in Nicolaou et al., “Molecular diagnosis of peanut and legume allergy,” Curr. Opin. Allergy Clin. Immunol., 11(3):222-8 (2011) and WO 2016205764, each of which is incorporated herein by reference in its entirety.

Delivery of CRISPR Systems

Through this disclosure and knowledge in the art, the CRISPR systems described herein, components thereof, nucleic acid molecules thereof, or nucleic acid molecules encoding or providing components thereof can be delivered by various delivery systems such as vectors, e.g., plasmids or viral delivery vectors. The CRISPR effectors and/or any of the RNAs (e.g., RNA guides) disclosed herein can be delivered using suitable vectors, e.g., plasmids or viral vectors, such as adeno-associated viruses (AAV), lentiviruses, adenoviruses, and other viral vectors, or combinations thereof. An effector and one or more RNA guides can be packaged into one or more vectors, e.g., plasmids or viral vectors.

In some embodiments, vectors, e.g., plasmids or viral vectors, are delivered to the tissue of interest by, e.g., intramuscular injection, intravenous administration, transdermal administration, intranasal administration, oral administration, or mucosal administration. Such delivery may be either via one dose or multiple doses. One skilled in the art understands that the actual dosage to be delivered herein may vary greatly depending upon a variety of factors, including, but not limited to, the vector choices, the target cells, organisms, tissues, the general conditions of the subject to be treated, the degrees of transformation/modification sought, the administration routes, the administration modes, and the types of transformation/modification sought.

In certain embodiments, delivery is via adenoviruses, which can be one dose containing at least 1×10⁵ particles (also referred to as particle units, pu) of adenoviruses. In some embodiments, the dose preferably is at least about 1×10⁶ particles, at least about 1×10⁷ particles, at least about 1×10⁸ particles, and at least about 1×10⁹ particles of the adenoviruses. The delivery methods and the doses are described, e.g., in WO 2016205764 and U.S. Pat. No. 8,454,972, each of which is incorporated herein by reference in its entirety.

In some embodiments, delivery is via plasmids. The dosage can be a sufficient number of plasmids to elicit a response. In some cases, suitable quantities of plasmid DNA in plasmid compositions can be from about 0.1 to about 2 mg. Plasmids will generally include (i) a promoter; (ii) a sequence encoding a nucleic acid-targeting CRISPR effector, operably linked to the promoter; (iii) a selectable marker; (iv) an origin of replication; and (v) a transcription terminator downstream of and operably linked to (ii). The plasmids can also encode the RNA components of a CRISPR complex, but one or more of these may instead be encoded on different vectors. The frequency of administration is within the ambit of the medical or veterinary practitioner (e.g., physician, veterinarian), or a person skilled in the art.

In another embodiment, delivery is via liposomes or lipofectin formulations or the like and can be prepared by methods known to those skilled in the art. Such methods are described, for example, in WO 2016205764, U.S. Pat. Nos. 5,593,972, 5,589,466, and 5,580,859, each of which is incorporated herein by reference in its entirety.

In some embodiments, delivery is via nanoparticles or exosomes. For example, exosomes have been shown to be particularly useful in delivery RNA.

Further means of introducing one or more components of the CRISPR systems described herein to a cell is by using cell-penetrating peptides (CPP). In some embodiments, a cell penetrating peptide is linked to a CRISPR effector. In some embodiments, a CRISPR effector and/or RNA guide is coupled to one or more CPPs for transportation into a cell (e.g., plant protoplasts). In some embodiments, the CRISPR effector and/or RNA guide(s) are encoded by one or more circular or non-circular DNA molecules that are coupled to one or more CPPs for cell delivery.

CPPs are short peptides of fewer than 35 amino acids derived either from proteins or from chimeric sequences capable of transporting biomolecules across cell membrane in a receptor independent manner. CPPs can be cationic peptides, peptides having hydrophobic sequences, amphipathic peptides, peptides having proline-rich and anti-microbial sequences, and chimeric or bipartite peptides. Examples of CPPs include, e.g., Tat (which is a nuclear transcriptional activator protein required for viral replication by HIV type 1), penetratin, Kaposi fibroblast growth factor (FGF) signal peptide sequence, integrin β3 signal peptide sequence, polyarginine peptide Args sequence, Guanine rich-molecular transporters, and sweet arrow peptide. CPPs and methods of using them are described, e.g., in Hällbrink et al., “Prediction of cell-penetrating peptides,” Methods Mol. Biol., 2015; 1324:39-58; Ramakrishna et al., “Gene disruption by cell-penetrating peptide-mediated delivery of Cas9 protein and guide RNA,” Genome Res., 2014 June;24(6):1020-7; and WO 2016205764, each of which is incorporated herein by reference in its entirety.

Various delivery methods for the CRISPR systems described herein are also described, e.g., in U.S. Pat. No. 8,795,965, EP 3009511, WO 2016205764, and WO 2017070605, each of which is incorporated herein by reference in its entirety.

### EXAMPLES

The invention is further described in the following examples, which do not limit the scope of the invention described in the claims.

### Example 1—Identification of Components of CLUST.133120 CRISPR-Cas System

This protein family was identified using the computational methods described above. The CLUST.133120 system comprises single effectors associated with CRISPR systems found in uncultured metagenomic sequences collected from freshwater, wastewater, soil, and rhizosphere environments (TABLE 2). Exemplary CLUST.133120 effectors include those shown in TABLES 2 and 3, below. Examples of direct repeat sequences for these systems are shown in TABLE 4. Optionally, the system includes a tracrRNA that is contained in a non-coding sequence listed in TABLE 5.

TABLE 2
Representative CLUST.133120 Effector Proteins

| | | # | effector | SEQ |
|---|---|---|---|---|
| source | effector accession | spacers | size (aa) | ID NO |
| terrestrial-soil | 3300027740\|Ga0214474_1000103_17\|M | 3 | 498 | 1 |
| aquatic-non marine saline | 3300017971\|Ga0180438_10084474_2\|M | 4 | 517 | 2 |
| and alkaline-hypersaline | | | | |
| lake sediment | | | | |
| aquatic-freshwater | 3300022592\|Ga0236342_1001376_7\|P | 4 | 756 | 3 |
| aquatic-freshwater- | 3300020171\|Ga0180732_1000630_46\|P | 6 | 487 | 4 |
| groundwater | | | | |
| aquatic-freshwater- | 3300018040\|Ga0187862_10024620_1\|M | 9 | 580 | 5 |
| peatland | | | | |
| aquatic-marine-marine | 3300005253\|Ga0073583_1150562_58\|P | 20 | 511 | 6 |
| sediment | | | | |
| aquatic-marine-marine | 3300005253\|Ga0073583_1150562_51\|M | 20 | 492 | 7 |
| sediment | | | | |
| marine sediment | LAZR01002400_19\|P | 20 | 511 | 8 |
| metagenome | | | | |
| marine sediment | LAZR01002400_15\|M | 20 | 492 | 9 |
| metagenome | | | | |
| marine sediment | SDBU01080949_7\|P | 2 | 511 | 10 |
| metagenome | | | | |
| marine sediment | SDBU01080949_6\|M | 2 | 492 | 11 |
| metagenome | | | | |
| marine sediment | SDBV01087475_1\|P | 17 | 511 | 12 |
| metagenome | | | | |
| marine sediment | SDBV01087475_1\|M | 17 | 492 | 13 |
| metagenome | | | | |
| marine sediment | SRR2657585_2135055_9\|M | 11 | 484 | 14 |
| metagenome | | | | |
| marine sediment | SRR2657585_2135055_8\|P | 11 | 506 | 15 |
| metagenome | | | | |
| marine sediment | SRR2657585_1667169_1\|M | 4 | 532 | 16 |
| metagenome | | | | |
| marine sediment | SRR8451930_3283449_6\|P | 8 | 511 | 17 |
| metagenome | | | | |
| marine sediment | SRR8451930_3283449_6\|M | 8 | 492 | 18 |
| metagenome | | | | |
| marine sediment | SRR8452060_665073_11\|P | 17 | 511 | 19 |
| metagenome | | | | |
| marine sediment | SRR8452060_665073_10\|M | 17 | 492 | 20 |
| metagenome | | | | |
| microbial mat metagenome | SRR6448207_2353026_47\|M | 3 | 605 | 21 |
| peat metagenome | SRR7004342_2720286_9\|P | 19 | 645 | 22 |
| peat metagenome | SRR7004342_2720286_9\|M | 19 | 616 | 23 |
| permafrost metagenome | SRR6491179_278020_2\|M | 5 | 733 | 24 |
| plants-rhizosphere-carex | 3300028800\|Ga0265338_10000470_38\|P | 58 | 651 | 25 |
| aquatilis | | | | |
| plants-rhizosphere-carex | 3300031235\|Ga0265330_10003750_4\|P | 39 | 651 | 26 |
| aquatilis | | | | |
| plants-rhizosphere-carex | 3300031241\|Ga0265325_10000012_120\|M | 58 | 651 | 27 |
| aquatilis | | | | |
| plants-rhizosphere-carex | 3300031242\|Ga0265329_10001065_5\|P | 44 | 651 | 28 |
| aquatilis | | | | |
| plants-rhizosphere-carex | 3300031247\|Ga0265340_10001032_18\|P | 29 | 651 | 29 |
| aquatilis | | | | |
| plants-rhizosphere-carex | 3300031250\|Ga0265331_10001842_6\|P | 44 | 651 | 30 |
| aquatilis | | | | |
| plants-rhizosphere-carex | 3300031344\|Ga0265316_10000001_557\|P | 53 | 651 | 31 |
| aquatilis | | | | |
| plants-rhizosphere-spartina | 3300031733\|Ga0316577_10033675_3\|M | 7 | 505 | 32 |
| alterniflora | | | | |
| rhizosphere metagenome | SRR8543844_2229942_334\|P | 58 | 651 | 33 |
| rhizosphere metagenome | SRR8543847_1991039_375\|P | 58 | 651 | 34 |
| rhizosphere metagenome | SRR8543989_2137428_34\|M | 58 | 651 | 35 |
| rhizosphere metagenome | SRR8543995_2079833_6\|M | 51 | 651 | 36 |
| rhizosphere metagenome | SRR8544146_618080_5\|P | 24 | 651 | 37 |
| sediment metagenome | SRR6963591_1323413_2\|M | 3 | 517 | 38 |
| soil metagenome | SRR7976138_3273187_71\|P | 20 | 571 | 39 |
| soil metagenome | SRR7976138_3273187_70\|M | 20 | 582 | 40 |
| terrestrial-soil | 3300012943\|Ga0164241_10000441_40\|P | 20 | 571 | 41 |
| terrestrial-soil | 3300012943\|Ga0164241_10000441_39\|M | 20 | 582 | 42 |
| terrestrial-soil | 3300031949\|Ga0214473_10000008_352\|M | 53 | 691 | 43 |
| terrestrial-soil | 3300031965\|Ga0326597_10001868_12\|M | 9 | 404 | 44 |
| terrestrial-soil-arctic peat | 3300006795\|Ga0075520_1037237_1\|M | 4 | 559 | 45 |
| soil | | | | |
| terrestrial-soil-bog | 3300014491\|Ga0182014_10039677_1\|P | 5 | 733 | 46 |
| terrestrial-soil-bog | 3300014491\|Ga0182014_10071250_1\|M | 2 | 559 | 47 |
| terrestrial-soil-tropical | 3300017975\|Ga0187782_10004712_9\|P | 20 | 645 | 48 |
| peatland | | | | |
| terrestrial-soil-tropical | 3300017975\|Ga0187782_10004712_9\|M | 20 | 616 | 49 |
| peatland | | | | |
| wetland metagenome | SRR7027835_762984_1\|M | 9 | 580 | 50 |

TABLE 3
Amino acid sequences of Representative CLUST.133120 Effector Proteins

>3300027740|Ga0214474_1000103_17|M
[terrestrial-soil]
MTTKVFKYGALPVTLDSEIRKKMKLAREYYNQLVEAENGRRKTVWGGERPKQPPHEHKDEPVCEECKKHWKDIRDKYFATPPLDIK
PLRAKASEAGLYWGTYLIIEESFSAAWKKTDGLSTVRYKSWRDGGIMGVQLQQAHSANGFYRIEQAPDPRKGRKAGQRHAVKIRIG
TRENKPVWSEPIAFEMHRPLQGRPTWVKICYFYRGEREIWSVNITCTDVPERALPDTAKVLAIDIGWRVMSDDSIRAAYARDENGN
ESELLFDSRWRECGDRADRIRSARDDRLNELKKKDTRFSLIKKPSGIRGYVAKNSIEDTEVTEWIKQDRHLEQYELGCRRRSVAVR
KEKLRVWLSSLRKQYATAIIKDSSHKEMKAAKRAKADGMPPPARRNAHHGAPGEIVEEICRAFGRQTNIAIVEAPGTTATCASCGQ
EMEIGAELMITCERCNAREDRDCISTRNMLRLYASGNCKKPTARKTIARFAKRHKNYVEPNQCQRDGL (SEQ ID NO: 1)
>3300017971|Ga0180438_10084474_2|M
[aquatic-non marine saline and alkaline-hypersaline lake sediment]
MKVFKYGARPVNQESLIRDQMKLARNYYNKLVETENKRRQQAWGSEKPPPPPHPPEEVVLPDGSKKTKWCRCEECKTHWKTLREAY
YALDNLDKKPLRAAASSWGLYWGSYLLVEQAFDQAQKTTVYYDPVRFRGWRKGGQAGLQIQNGKNTDSLFKVEEIPDPEPSARSKK
NDRRAGKGKRRGQLRTLRIRIGTEGRTPIWSDPIIFKMHRPLEGTVCWLKICMFYRGDREIWSVNFTCKDTSPRTDSATDGVVAID
VSWRQLPNGDIRLAYARGDNGFEDELTLDNSWLERIKKAERIQSHRDERLNDLKTSDSRFRLFKSPRSARIHIMKNQIAELEDWAK
REKHLEQYELGCKRKTYDVRRDAVRVWLRKLRRLYQTVVIKDSSHKEMKEQAKAKKKLHRAARKQGHHGAPGEIVEEICKVFGRIE
NVAVVIAENTTATCPVCGHMHEVNKERMITCERCGTTDDRDRISTHNLLSRFFDGQYEKPTARKTESKFAKKHKKVEQEVKHLRDG
L (SEQ ID NO: 2)
>3300022592|Ga0236342_1001376_7|P
[aquatic-freshwater]
MIKLLTYDGDCVFVHAGDTTLKTTKAFTHGTIVFDEKARTVTFGKTDAVALPDDLSLKENATLNFQLVAPDGTLRKQVRSGILKAN
EKQPGLLHISGMAKSPRRYAAEDGWGNAIYKYRAYFTHPGLNTDGELPEWLKGSIHRQKAFWNQLAWLCRDARRNCSPVPTEEIIA
FVQQTILPEIDAFNDSLGRSKDKMKHPAKLKKEDPGIDNLWHFVGELRTRIAKGRAVPDGLLEKVITFAQQYKPDYTPLNEFLNHF
TEIAETEAAALKLRRFEIRPTMTAFRASLNRRQTNKSTWSEGWPLIKYPDSPKADDWSLNYYLNKAAVDAAGLESGVNVGGLSFGP
ALEPAATGHANLHGDAAKRKMREAEISILGPDKEPWKFRFGVLQHRPLPENSHIKEWKLLFHNGALWLDIVVELQRPLPATGGLTA
GLEIGWRRTEEGIRFGTLYEPVRSTIRELTIDLQRSPKDQKDRVPFRIDMGPNRWEKRNIARLFLKHKPGEELKFSKASDSIRQIA
ALFPDWKAGDMVPNMLGVRDVLQRRRDAAMQTTKIQMRQHLGEQTPAWLDKAGRRGLLRLQEELKDDAKAQAILNPWWVNDEVIGK
LARFYTARSVRRMEYGQMQVAHDVCRYLRDNGVSRLIVEAKFLAKVAQQQDSNDPVVLQLSQKYRQFSASAKFLAFLKNTAAKYGI
VIDTHVALNISRICHYCDHLNPPTAKESYQCEKCKRVVKQDPNAAINLARFGSDPELAEMAAQAGRVE (SEQ ID NO: 3)
>3300020171|Ga0180732_1000630_46|P
[aquatic-freshwater-groundwater]
MRLGRECYNALVEVENARRHLAWGGDIPPKPPHPLPLTODGDENDDETAWCRCAECKAHWMGIREALHALPPIDTKPIYAHYCTNG
PLYWGTFNHVAQAFSAAWKKTRPWRPVKYRSWRQGGVIAVQIQAGYQKRQNYDTLFRINDVVDTRTRHRHKGHHRATVQLALGAGR
MTSPIEIEQHRPIVGTPTWVTARLCYDSLGRERAAVNFTRRDVPQRTDLAPRGVVAVDVSWRQVPDGLRIAWTRDDQGHNDQLVLD
TRWQFLAKHADQLQAIRDQNLNLLKAVDPAFAQAKSPRHACLILAEIEDPSAVARAWRYREEHLRNYEMGERRHSVNRRRDALRKW
ARELRRNYAEVVVKDSQHKEMKETAKQDKLSRKARRQGQHGAPGEVIAVLREIFGDDGMWLVEAAYTTDRCPLCGHLNAHGAERIV
VCEECGEGLDRDLVSTRNMLVAYAAEERRRPTARKTAARFGKRHKTSTSLDNASPAA (SEQ ID NO: 4)
>3300018040|Ga0187862_10024620_1|M
[aquatic-freshwater-peatland]
MAWLCRETRLKCSPVPTDKIVDFVQNAILPEIDAFNSAPGRSRERMKHPAKLKIETPGLDCLWGFVGELRARTGKGRAVPAGLLDK
VVEFAQQFKTDYTPFNEFIAGFQAIAEREAAALGLRHFEIRPTVGAFKAVLDRRKTTKAHWSEGWPLIKYPDSPKAANWGLHYHLN
KAGVDSALLEREEGIPGLSFGPRLKPADTGHEKLTGVAAKRILREAQISISGDNDERWTFHFGVLQHRPLPPNSHLKEWKLIFQGG
ALWLCLVVELQRPVPVPGPHPAGLDIGWRRTEEGIRFGTLYEPATKTIRELTVDLQKSPCDHKDRVPFRIDLGPSRWEMRNATELV
PDWKPGQPIPSAFETRSVLQIRRDRIKETAKRQLREHLGERLPVWFDKAGRRGLLKLAQDFKNESAVCEILNIWQQKDEQLGKLAS
MYFDRSTKRIEYGHAQVAHDVCRFLQQKGVMRLIVETSFLSKVSQHQDNEDLVSIERSQKYRQFVAVGKFLAALKNTTRKYGIVVE
TVNAMNTTRTCQYCNALNPRTEEEQFTCKRCGRQVQQDHNAAVNLSRFGSDPVLAEMALHADEV (SEQ ID NO: 5)
>3300005253|Ga0073583_1150562_58|P
[aquatic-marine-marine sediment]
MTKVYKYGALPGGDTLCAQMNLGRVYYNSLVEAENERRTTMWGGDRPPSPATHVCKKSCSTCDKAESKKRKPRKHECKKFCPVCRA
HYKALRKQYRSEPPLDVKPFRKKAAEGGLYWGTYLVIEQDFSAAWKETESFSLVKFRSWRQGDMCAVQIQRDKDPDRMFLIKSAPD
PRKKKQQRYTLRLRVGSKGQAPVWAEPLPFEMHRPLQGTATWVKIARKYVADRVIWSVQFTRRDIPERKDNAERGAVAIDVGWRKT
DDGMRIAYARGDDGAEYELVLPPKWMKHADQADRIRSARDQNLVELQKQERFWSVILAVCGFSNKKLFARLKSTLSVRRVAKPGEH
TKWIKKERHLWQYEAGCRNRSVTRRRNDVRVWLRDLRRRYAHAVIKDSCHKKMKENKTSLPKPARRQGHHAAPGEVIEEITRVFGR
ITGVSVVCAVDTTNHCPACSFVNSYGPERVVTCGGCGVVEDRDRVSTQNMMNMYAIGNVRNPTTRKSTPRFAKKHKDPEAP (SEQ
ID NO: 6)
>3300005253|Ga0073583_1150562_51|M
[aquatic-marine-marine sediment]
MNLGRVYYNSLVEAENERRTTMWGGDRPPSPATHVCKKSCSTCDKAESKKRKPRKHECKKFCPVCRAHYKALRKQYRSEPPLDVKP
FRKKAAEGGLYWGTYLVIEQDFSAAWKETESFSLVKFRSWRQGDMCAVQIQRDKDPDRMFLIKSAPDPRKKKQQRYTLRLRVGSKG
QAPVWAEPLPFEMHRPLQGTATWVKIARKYVADRVIWSVQFTRRDIPERKDNAERGAVAIDVGWRKTDDGMRIAYARGDDGAEYEL
VLPPKWMKHADQADRIRSARDQNLVELQKQERFWSVILAVCGFSNKKLFARLKSTLSVRRVAKPGEHTKWIKKERHLWQYEAGCRN
RSVTRRRNDVRVWLRDLRRRYAHAVIKDSCHKKMKENKTSLPKPARRQGHHAAPGEVIEEITRVFGRITGVSVVCAVDTTNHCPAC
SFVNSYGPERVVTCGGCGVVEDRDRVSTQNMMNMYAIGNVRNPTTRKSTPRFAKKHKDPEAP (SEQ ID NO: 7)
>LAZR01002400_19|P
[marine sediment metagenome]
MTKVYKYGALPGGDTLCAQMNLGRVYYNSLVEAENERRTTMWGGDRPPSPATHVCKKSCSTCDKAESKKRKPRKHECKKFCPVCRA
HYKALRKQYRSEPPLDVKPFRKKAAEGGLYWGTYLVIEQDFSAAWKETESFSLVKFRSWRQGDMCAVQIQRDKDPDRMFLIKSAPD
PRKKKQQRYTLRLRVGSKGQAPVWAEPLPFEMHRPLQGTATWVKIARKYVADRVIWSVQFTRRDIPERKDNAERGAVAIDVGWRKT
DDGMRIAYARGDDGAEYELVLPPKWMKHADQADRIRSARDQNLVELQKQERFWSVILAVCGFSNKKLFARLKSTLSVRRVAKPGEH
TKWIKKERHLWQYEAGCRNRSVTRRRNDVRVWLRDLRRRYAHAVIKDSCHKKMKENKTSLPKPARRQGHHAAPGEVIEEITRVFGR
ITGVSVVCAVDTTNHCPACSFVNSYGPERVVTCGGCGVVEDRDRVSTQNMMNMYAIGNVRNPTTRKSTPRFAKKHKDPEAP (SEQ
ID NO: 8)
>LAZR01002400_15|M
[marine sediment metagenome]
MNLGRVYYNSLVEAENERRTTMWGGDRPPSPATHVCKKSCSTCDKAESKKRKPRKHECKKFCPVCRAHYKALRKQYRSEPPLDVKP
FRKKAAEGGLYWGTYLVIEQDFSAAWKETESFSLVKFRSWRQGDMCAVQIQRDKDPDRMFLIKSAPDPRKKKQQRYTLRLRVGSKG
QAPVWAEPLPFEMHRPLQGTATWVKIARKYVADRVIWSVQFTRRDIPERKDNAERGAVAIDVGWRKTDDGMRIAYARGDDGAEYEL
VLPPKWMKHADQADRIRSARDQNLVELQKQERFWSVILAVCGFSNKKLFARLKSTLSVRRVAKPGEHTKWIKKERHLWQYEAGCRN
RSVTRRRNDVRVWLRDLRRRYAHAVIKDSCHKKMKENKTSLPKPARRQGHHAAPGEVIEEITRVFGRITGVSVVCAVDTTNHCPAC
SFVNSYGPERVVTCGGCGVVEDRDRVSTQNMMNMYAIGNVRNPTTRKSTPRFAKKHKDPEAP (SEQ ID NO: 9)
>SDBU01080949_7|P
[marine sediment metagenome]
MTKVYKYGALPGGDTLCAQMNLGRVYYNSLVEAENERRTTMWGGDRPPSPATHVCKKSCSTCDKAESKKRKPRKHECKKFCPVCRA
HYKALRKQYRSEPPLDVKPFRKKAAEGGLYWGTYLVIEQDFSAAWKETESFSLVKFRSWRQGDMCAVQIQRDKDPDRMFLIKSAPD
PRKKKQQRYTLRLRVGSKGQAPVWAEPLPFEMHRPLQGTATWVKIARKYVADRVIWSVQFTRRDIPERKDNAERGAVAIDVGWRKT
DDGMRIAYARGDDGAEYELVLPPKWMKHADQADRIRSARDQNLVELQKQERFWSVILAVCGFSNKKLFARLKSTLSVRRVAKPGEH
TKWIKKERHLWQYEAGCRNRSVTRRRNDVRVWLRDLRRRYAHAVIKDSCHKKMKENKTSLPKPARRQGHHAAPGEVIEEITRVFGR
ITGVSVVCAVDTTNHCPACSFVNSYGPERVVTCGGCGVVEDRDRVSTQNMMNMYAIGNVRNPTTRKSTPRFAKKHKDPEAP (SEQ
ID NO: 10)
>SDBU01080949_6|M
[marine sediment metagenome]
MNLGRVYYNSLVEAENERRTTMWGGDRPPSPATHVCKKSCSTCDKAESKKRKPRKHECKKFCPVCRAHYKALRKQYRSEPPLDVKP
FRKKAAEGGLYWGTYLVIEQDFSAAWKETESFSLVKFRSWRQGDMCAVQIQRDKDPDRMFLIKSAPDPRKKKQQRYTLRLRVGSKG
QAPVWAEPLPFEMHRPLQGTATWVKIARKYVADRVIWSVQFTRRDIPERKDNAERGAVAIDVGWRKTDDGMRIAYARGDDGAEYEL
VLPPKWMKHADQADRIRSARDQNLVELQKQERFWSVILAVCGFSNKKLFARLKSTLSVRRVAKPGEHTKWIKKERHLWQYEAGCRN
RSVTRRRNDVRVWLRDLRRRYAHAVIKDSCHKKMKENKTSLPKPARRQGHHAAPGEVIEEITRVFGRITGVSVVCAVDTTNHCPAC
SFVNSYGPERVVTCGGCGVVEDRDRVSTQNMMNMYAIGNVRNPTTRKSTPRFAKKHKDPEAP (SEQ ID NO: 11)
>SDBV01087475_1|P
[marine sediment metagenome]
MTKVYKYGALPGGDTLCAQMNLGRVYYNSLVEVENERRTTMWGGDRPPSPATHVCKKSCSTCDKAESKKRKPRKHECKKFCPVCRA
HYKALRKQYRSEPPLDVKPFRKKAAEGGLYWGTYLVIEQDFSAAWKETESFSLVKFRSWRQGDMCAVQIQRDKDPDRMFLIKSAPD
PRKKKQQRYTLRLRVGSKGQAPVWAEPLPFEMHRPLQGTATWVKIARKYVADRVIWSVQFTRRDIPERKDNAERGAVAIDVGWRKT
DDGMRIAYARGDDGAEYELVLPPKWMKHADQADRIRSARDQNLVELQKQERFWSVILAVCGFSNKKLFARLKSTLSVRRVAKPGEH
TKWIKKERHLWQYEAGCRNRSVTRRRNDVRVWLRDLRRRYAHAVIKDSCHKKMKENKTSLPKPARRQGHHAAPGEVIEEITRVFGR
ITGVSVVCAVDTTNHCPACSFVNSYGPERVVTCGGCGVVEDRDRVSTQNMMNMYAIGNVRNPTTRKSTPRFAKKHKDPEAP (SEQ
ID NO: 12)
>SDBV01087475_1|M
[marine sediment metagenome]
MNLGRVYYNSLVEVENERRTTMWGGDRPPSPATHVCKKSCSTCDKAESKKRKPRKHECKKFCPVCRAHYKALRKQYRSEPPLDVKP
FRKKAAEGGLYWGTYLVIEQDFSAAWKETESFSLVKFRSWRQGDMCAVQIQRDKDPDRMFLIKSAPDPRKKKQQRYTLRLRVGSKG
QAPVWAEPLPFEMHRPLQGTATWVKIARKYVADRVIWSVQFTRRDIPERKDNAERGAVAIDVGWRKTDDGMRIAYARGDDGAEYEL
VLPPKWMKHADQADRIRSARDQNLVELQKQERFWSVILAVCGFSNKKLFARLKSTLSVRRVAKPGEHTKWIKKERHLWQYEAGCRN
RSVTRRRNDVRVWLRDLRRRYAHAVIKDSCHKKMKENKTSLPKPARRQGHHAAPGEVIEEITRVFGRITGVSVVCAVDTTNHCPAC
SFVNSYGPERVVTCGGCGVVEDRDRVSTQNMMNMYAIGNVRNPTTRKSTPRFAKKHKDPEAP (SEQ ID NO: 13)
>SRR2657585_2135055_9|M
[marine sediment metagenome]
MKLGREYYNALVEVENKRRQKAWGAETVPAPPHEDCKLPKCLECRDHWRAIRKRVRDEPLIDFKPLRAEFADRGLYWGTYLCIEDA
FARAWKDRDSLRLVKFRSWRHGGIAGVQIQKATWQRSMGTTMFETASAGDTRTGRRARFGGRRVARLRIGSEDRAPLWCEDVPYEQ
HRAIEGRVTWVKIAMKYRGDREIWSVTFTCSDVPERPASDEAARGVVAVDVGWRRIDDDLRIAYARSDQGVVSELRMGPRWRELWE
RADRIRGHRDDHHNELLASGVEVPGQSRSANGLRSDIEKLIAAGENVGPELLAWMHRDRHLAQYETGCRRRSVDCRTDAMRKWARE
LRRTYATVVLKKTSTKKQKETAKENGLVPPARRQGQHAAPGEVLEYLTTTFGRDRTFLVKSKDTTNQCCRCGHVNNHGPETIITCE
QCGDAIDRDEASTQNMMELWVASECEEPTARKTTARFVKRHRKEGSPDNASPPA (SEQ ID NO: 14)
>SRR2657585_2135055_8|P
[marine sediment metagenome]
MATRVYKFGARSPGEATLLRAQMKLGREYYNALVEVENKRRQKAWGAETVPAPPHEDCKLPKCLECRDHWRAIRKRVRDEPLIDFK
PLRAEFADRGLYWGTYLCIEDAFARAWKDRDSLRLVKFRSWRHGGIAGVQIQKATWQRSMGTTMFETASAGDTRTGRRARFGGRRV
ARLRIGSEDRAPLWCEDVPYEQHRAIEGRVTWVKIAMKYRGDREIWSVTFTCSDVPERPASDEAARGVVAVDVGWRRIDDDLRIAY
ARSDQGVVSELRMGPRWRELWERADRIRGHRDDHHNELLASGVEVPGQSRSANGLRSDIEKLIAAGENVGPELLAWMHRDRHLAQY
ETGCRRRSVDCRTDAMRKWARELRRTYATVVLKKTSTKKQKETAKENGLVPPARRQGQHAAPGEVLEYLTTTFGRDRTFLVKSKDT
TNQCCRCGHVNNHGPETIITCEQCGDAIDRDEASTQNMMELWVASECEEPTARKTTARFVKRHRKEGSPDNASPPA (SEQ ID
NO: 15)
>SRR2657585_1667169_1|M
[marine sediment metagenome]
MKVYKFGALRPTENLDKFFEQTQMAHALYQRMVEERNAEREAALEPFKPFSPPHENCYEKRLAAAREKSKQTKKRVKIKRCKKCDD
YWIPIYKEAYKTLEFYKKPTCKEMTAEGLYWGTYQLIAEAFDRADDDTKLMRSIKYRKWPTVKHAVRVQIQDPDKAESYFRVEVVP
DRFDRWREERKRPMPWRVLWIRVGSDGRKPIWLKVPYKEHRPIDGRVTHVTVQRYFIADRERWEVLFTCKSEAERRDSATKGLVAV
DVGWRKMDDGSIRCAYAQDVEGNVSELRLPPEWIERITRADRIRGHRDENLNALLETAGSSLTHGIRSWAGVRRAYRRHLEAGGEK
IAEVEEALKRDRHLWQYELGNRVRAERRRRNDFRVWARELRRCYAVIVTKNTSHKEVKEKANKGPKKLPQKARNQGQHAAPGELIE
TLRRVFGWEGPKITGNALEVSAENTSRICAKCFHENEGSEKRIVVCEKCGVKDDRDHRSTRNMLRDVAAMKVTFGTARKRPSKYAN
RHKKKESSSNASETAS (SEQ ID NO: 16)
>SRR8451930_3283449_6|P
[marine sediment metagenome]
MTKVYKYGALPGGDTLCAQMNLGRVYYNSLVEVENERRTTMWGGDRPPSPATHVCKKSCSTCDKAESKKRKPRKHECKKFCPVCRA
HYKALRKQYRSEPPLDVKPFRKKAAEGGLYWGTYLVIEQDFSAAWKETESFSLVKFRSWRQGDMCAVQIQRDKDPDRMFLIKSAPD
PRKKKQQRYTLRLRVGSKGQAPVWAEPLPFEMHRPLQGTATWVKIARKYVADRVIWSVQFTRRDIPERKDNAERGAVAIDVGWRKT
DDGMRIAYARGDDGAEYELVLPPKWMKHADQADRIRSARDQNLVELQKQERFWSVILAVCGFSNKKLFARLKSTLSVRRVAKPGEH
TKWIKKERHLWQYEAGCRNRSVTRRRNDVRVWLRDLRRRYAHAVIKDSCHKKMKENKTSLPKPARRQGHHAAPGEVIEEITRVFGR
ITGVSVVCAVDTTNHCPACSFVNSYGPERVVTCGGCGVVEDRDRVSTQNMMNMYAIGNVRNPTTRKSTPRFAKKHKDPEAP (SEQ
ID NO: 17)
>SRR8451930_3283449_6|M
[marine sediment metagenome]
MNLGRVYYNSLVEVENERRTTMWGGDRPPSPATHVCKKSCSTCDKAESKKRKPRKHECKKFCPVCRAHYKALRKQYRSEPPLDVKP
FRKKAAEGGLYWGTYLVIEQDFSAAWKETESFSLVKFRSWRQGDMCAVQIQRDKDPDRMFLIKSAPDPRKKKQQRYTLRLRVGSKG
QAPVWAEPLPFEMHRPLQGTATWVKIARKYVADRVIWSVQFTRRDIPERKDNAERGAVAIDVGWRKTDDGMRIAYARGDDGAEYEL
VLPPKWMKHADQADRIRSARDQNLVELQKQERFWSVILAVCGFSNKKLFARLKSTLSVRRVAKPGEHTKWIKKERHLWQYEAGCRN
RSVTRRRNDVRVWLRDLRRRYAHAVIKDSCHKKMKENKTSLPKPARRQGHHAAPGEVIEEITRVFGRITGVSVVCAVDTTNHCPAC
SFVNSYGPERVVTCGGCGVVEDRDRVSTQNMMNMYAIGNVRNPTTRKSTPRFAKKHKDPEAP (SEQ ID NO: 18)
>SRR8452060_665073_11|P
[marine sediment metagenome]
MTKVYKYGALPGGDTLCAQMNLGRVYYNSLVEAENERRTTMWGGDRPPSPATHVCKKSCSTCDKAESKKRKPRKHECKKFCPVCRA
HYKALRKQYRSEPPLDVKPFRKKAAEGGLYWGTYLVIEQDFSAAWKETESFSLVKFRSWRQGDMCAVQIQRDKDPDRMFLIKSAPD
PRKKKQQRYTLRLRVGSKGQAPVWAEPLPFEMHRPLQGTATWVKIARKYVADRVIWSVQFTRRDIPERKDNAERGAVAIDVGWRKT
DDGMRIAYARGDDGAEYELVLPPKWMKHADQADRIRSARDQNLVELQKQERFWSVILAVCGFSNKKLFARLKSTLSVRRVAKPGEH
TKWIKKERHLWQYEAGCRNRSVTRRRNDVRVWLRDLRRRYAHAVIKDSCHKKMKENKTSLPKPARRQGHHAAPGEVIEEITRVFGR
ITGVSVVCAVDTTNHCPACSFVNSYGPERVVTCGGCGVVEDRDRVSTQNMMNMYAIGNVRNPTTRKSTPRFAKKHKDPEAP (SEQ
ID NO: 19)
>SRR8452060_665073_10|M
[marine sediment metagenome]
MNLGRVYYNSLVEAENERRTTMWGGDRPPSPATHVCKKSCSTCDKAESKKRKPRKHECKKFCPVCRAHYKALRKQYRSEPPLDVKP
FRKKAAEGGLYWGTYLVIEQDFSAAWKETESFSLVKFRSWRQGDMCAVQIQRDKDPDRMFLIKSAPDPRKKKQQRYTLRLRVGSKG
QAPVWAEPLPFEMHRPLQGTATWVKIARKYVADRVIWSVQFTRRDIPERKDNAERGAVAIDVGWRKTDDGMRIAYARGDDGAEYEL
VLPPKWMKHADQADRIRSARDQNLVELQKQERFWSVILAVCGFSNKKLFARLKSTLSVRRVAKPGEHTKWIKKERHLWQYEAGCRN
RSVTRRRNDVRVWLRDLRRRYAHAVIKDSCHKKMKENKTSLPKPARRQGHHAAPGEVIEEITRVFGRITGVSVVCAVDTTNHCPAC
SFVNSYGPERVVTCGGCGVVEDRDRVSTQNMMNMYAIGNVRNPTTRKSTPRFAKKHKDPEAP (SEQ ID NO: 20)
>SRR6448207_2353026_47|M
[microbial|Mat metagenome]
MTTTKVLKFWCEPADDDSKELLMQQISLGYTYARSLDDAENTRRRSFRDHLMDVTPFKEAENELTALYESTSKENRDDAFRTKVEL
LRSAKKDVYISSKTTEPFVSKIKEDQDRYRAEVRRIRADLGPRGHGLSFGSYQHEEDAHLQSKSTRAPYLPLIRKRLPKEGTFAVH
LQSEYKAPIQNVLDGSCRWVSIGSEMYSISRKVKYGSRTRDDSKSRGRRLRQVSLRVSGKGGDTKLIKVHALIHRMPVTGTVVWAR
IHTQRTGRKLRYSLQISVDNPVFPADVKNIRTAKSGVIGIDLGWRETDEGFLIATASEQNGNQLPPLIIPKEVALRGITHQRIDSR
CETLQSTRDQKFNDIINRIRHVRDSAADWFREHTQHCHKWRSPGKVVGLFSKWRNNRWNGDDEIFDATSAFIKKDGHLWDWEAHNR
TRMSRQIRGTYQRWADILASNYAEIRIENLDIKQAAEDFRKKAESEHQKDNNKSKSPLIDTRAKLQLAHVGHSLVRSCNRNECNLG
RVDKRYTSRKCHLCGTKGKKTSDKLVFCKNKECALYNCAVDRDIRAALNIASSEVLEWVAGPLAAPKEPKERKNPRRRTKQAETIS
AAA (SEQ ID NO: 21)
>SRR7004342_2720286_9|P
[peat metagenome]
MATKVLKFYCVPASARDNQLLCEQVKLGSMYRRDLAWIENRARELRRAIADWPARDVPIEKRSEWWKSDVGRAASKAWHESDECKR
LKKSITDGQIQAATAARKKARRFDTAWGTCGLAAEAAEAARVALEKEFGLIDKETGQTTFVNTKFPSDEGRVGVQFQRDTDKIDEE
TGEVIPAKKPTIYADELIGGSHNFCRIGSARYSLLDRVDGFQPAALDASGAPIDSFGYKSKQPKGKRFHLLQIRVGTVDKRSTAYP
FDLDPDKGEPIWAKLHVLLHQKTKRPQTLPHERVKWVFVQRVRCALRYRWSVCFVIEDSVVQKPHEQPNDRVAVDLGWRQLFDESG
TPAGIRLLYWMATSPLDLNDPSSPVEGQLVIPQHVVDRKPFSSQLESNRTKNREAMQAMLLAYLQSVSSATWLAKRTAQLASWKKP
SKFVLLLNEWRKNRIAGDQAAFAALEAWNKQDAHLYLWSAPNITKMQRQIQGRVDQFAVQLARRYGIVIVENFKLPDVIEKKDLQE
QDQDAQKLRKKNARRVNVVAPGRARAALKRFATKYNSHYIEEESAFTTVDCASCGHRREFDLKNARAQLLLACDNCGVVEDQDRTA
ARNLLAGASAIARGENGWPLEAKVSKASKKKVVLRRTRKRIVV (SEQ ID NO: 22)
>SRR7004342_2720286_9|M
[peat metagenome]
MYRRDLAWIENRARELRRAIADWPARDVPIEKRSEWWKSDVGRAASKAWHESDECKRLKKSITDGQIQAATAARKKARRFDTAWGT
CGLAAEAAEAARVALEKEFGLIDKETGQTTFVNTKFPSDEGRVGVQFQRDTDKIDEETGEVIPAKKPTIYADELIGGSHNFCRIGS
ARYSLLDRVDGFQPAALDASGAPIDSFGYKSKQPKGKRFHLLQIRVGTVDKRSTAYPFDLDPDKGEPIWAKLHVLLHQKTKRPQTL
PHERVKWVFVQRVRCALRYRWSVCFVIEDSVVQKPHEQPNDRVAVDLGWRQLFDESGTPAGIRLLYWMATSPLDLNDPSSPVEGQL
VIPQHVVDRKPFSSQLESNRTKNREAMQAMLLAYLQSVSSATWLAKRTAQLASWKKPSKFVLLLNEWRKNRIAGDQAAFAALEAWN
KQDAHLYLWSAPNITKMQRQIOGRVDQFAVOLARRYGIVIVENFKLPDVIEKKDLQEQDQDAQKLRKKNARRVNVVAPGRARAALK
RFATKYNSHYIEEESAFTTVDCASCGHRREFDLKNARAQLLLACDNCGVVEDQDRTAARNLLAGASAIARGENGWPLEAKVSKASK
KKWLRRTRKRIW (SEQ ID NO: 23)
>SRR6491179_278020_2|M
[permafrost metagenome]
MIDRLTFDGKCVYVHLAAISLKATKVFTHGGWVFDLAAHTVAFKDIPPVALPENLTLSDGASLTFQLVAPDGTLRKQVRSGVLKSN
DKQPGILRIAGRSKTPANFSAADGWKIEVFKYRAYFTHPGLKTEAGLPEWLQNSIKRQRDFWNRMAWLCRDARRRCSPVPTEEIVA
FVQESVLPAIDEFNNSLGIARSKEKIKHPINLKIEMPGLDAVWKFVGELRKRIAKNRGVPDGLLEKVVAFAEQFKADYGPLNEFIS
NLESIAKIESTALGLRQFEIRPTINSFKAVLDRRKTLKSAWSDGWPHIKYADSPRAADWGVYYYFNKSGVDSARLEAGPGVPGLTF
GRPLKPADTGHKSMKSPKRSSRALREAKISIAGDNREEWAFKFGVLQHRPLPPDSHLKQWSLIYSGGALWLCFTVERKRPLPFPGM
HAAGLDIGWRRTEEGIRFGILYEPESKTFRELSIDLQKSPEDHNNRMPFRIDMGPNRWDKRNITQLLPDWKPGDAIPNTVVVRRAL
GSRRSSYKDAAKVLLQKHLGDQLPVWFDKAGSRGLFKLKENFKDVPVVQEIIDDLQKKTEALNAVAGKYTARYTRELEYGQIQIAH
DVCRHLQQKGVTRLIVEASFVAKASQKQDNEDPASLKNSQKYRQFAAVSKFVSLLKNIAVKYGIAVEVLSAQNTTRICQYCNHLNP
STEKEIFFCEGCGKQVQQDQNAAVNLSRFGIDPELAEMALTFSKD (SEQ ID NO: 24)
>3300028800|Ga0265338_10000470_38|P
[plants-rhizosphere-carex aquatilis]
MTMRVYKFLCLPATAADDHCLREQVRLGARYRRQIALIENRERFLRRALIDYPARDVPVEARGAWWKSEAGKAARDAWHASDEFKA
LRERVFAGKKRALKAVRDATIEAGAMWGTLGKADDAADFARRTIDKAGLIDENGRTTFVGTDFPPDVGRIAVQFQRQNARIDARTG
KTLPAKKSVYADDLIGGEGSWLRIGSVAYSLGAPVDHFRPAAVDAEGRAIDVAGNPGDVIKIGPRAGEAATKGKRFHALAIRVGTV
PGTIRPIWAQMHLLLHQSPRTKNRLQLGHDVVKWAVVRRERTGLRYRWYLTLEVEGSVAVTTPHPHPHDAVAVHIGWRQLFDEEGK
KAGIRLLTWSATAPLDPNDPASPLWGQLVIPEDVLGVKPWADSIHSIRDQNQDVLRDMILAYARSLPATSWLREESRHMHNWRAPS
KYVRLHRQWRDQRIHGDQPAFAALEAWSKQDVHLLHREVPSLERMQRQVEGRVTAVAVQLAKRYGVVARDDFDVTGLVEKDDQHDE
DEQKLRKKTARRVNILGPGRARARVGYFAKKYGSVDLEVDAAGGTLDCAACGHHRDVSREDRVQRVIRCENCGHAEDQDITMSRNL
LAAASAVARGESGWPLDPKVPAPSTKKKAPARRNRRKDQQPLAPSPATP (SEQ ID NO: 25)
>3300031235|Ga0265330_10003750_4 P
[plants-rhizosphere-carex aquatilis]
MTMRVYKFLCLPATAADDHCLREQVRLGARYRRQIALIENRERFLRRALIDYPARDVPVEARGAWWKSEAGKAARDAWHASDEFKA
LRERVFAGKKRALKAVRDATIEAGAMWGTLGKADDAADFARRTIDKAGLIDENGRTTFVGTDFPPDVGRIAVQFQRQNARIDARTG
KTLPAKKSVYADDLIGGEGSWLRIGSVAYSLGAPVDHFRPAAVDAEGRAIDVAGNPGDVIKIGPRAGEAATKGKRFHALAIRVGTV
PGTIRPIWAQMHLLLHQSPRTKNRLQLGHDVVKWAVVRRERTGLRYRWYLTLEVEGSVAVTTPHPHPHDAVAVHIGWRQLFDEEGK
KAGIRLLTWSATAPLDPNDPASPLWGQLVIPEDVLGVKPWADSIHSIRDQNQDVLRDMILAYARSLPATSWLREESRHMHNWRAPS
KYVRLHRQWRDQRIHGDQPAFAALEAWSKQDVHLLHREVPSLERMQRQVEGRVTAVAVQLAKRYGVVARDDFDVTGLVEKDDQHDE
DEQKLRKKTARRVNILGPGRARARVGYFAKKYGSVDLEVDAAGGTLDCAACGHHRDVSREDRVQRVIRCENCGHAEDQDITMSRNL
LAAASAVARGESGWPLDPKVPAPSTKKKAPARRNRRKDQQPLAPSPATP (SEQ ID NO: 26)
>3300031241|Ga0265325_10000012_120|M
[plants-rhizosphere-carex aquatilis]
MTMRVYKFLCLPATAADDHCLREQVRLGARYRRQIALIENRERFLRRALIDYPARDVPVEARGAWWKSEAGKAARDAWHASDEFKA
LRERVFAGKKRALKAVRDATIEAGAMWGTLGKADDAADFARRTIDKAGLIDENGRTTFVGTDFPPDVGRIAVQFQRQNARIDARTG
KTLPAKKSVYADDLIGGEGSWLRIGSVAYSLGAPVDHFRPAAVDAEGRAIDVAGNPGDVIKIGPRAGEAATKGKRFHALAIRVGTV
PGTIRPIWAQMHLLLHQSPRTKNRLQLGHDVVKWAVVRRERTGLRYRWYLTLEVEGSVAVTTPHPHPHDAVAVHIGWRQLFDEEGK
KAGIRLLTWSATAPLDPNDPASPLWGQLVIPEDVLGVKPWADSIHSIRDQNQDVLRDMILAYARSLPATSWLREESRHMHNWRAPS
KYVRLHRQWRDQRIHGDQPAFAALEAWSKQDVHLLHREVPSLERMQRQVEGRVTAVAVQLAKRYGVVARDDFDVTGLVEKDDQHDE
DEQKLRKKTARRVNILGPGRARARVGYFAKKYGSVDLEVDAAGGTLDCAACGHHRDVSREDRVQRVIRCENCGHAEDQDITMSRNL
LAAASAVARGESGWPLDPKVPAPSTKKKAPARRNRRKDQQPLAPSPATP (SEQ ID NO: 27)
>3300031242 Ga0265329_10001065_5|P
[plants-rhizosphere-carex aquatilis]
MTMRVYKFLCLPATAADDHCLREQVRLGARYRRQIALIENRERFLRRALIDYPARDVPVEARGAWWKSEAGKAARDAWHASDEFKA
LRERVFAGKKRALKAVRDATIEAGAMWGTLGKADDAADFARRTIDKAGLIDENGRTTFVGTDFPPDVGRIAVQFQRQNARIDARTG
KTLPAKKSVYADDLIGGEGSWLRIGSVAYSLGAPVDHFRPAAVDAEGRAIDVAGNPGDVIKIGPRAGEAATKGKRFHALAIRVGTV
PGTIRPIWAQMHLLLHQSPRTKNRLQLGHDVVKWAVVRRERTGLRYRWYLTLEVEGSVAVTTPHPHPHDAVAVHIGWRQLFDEEGK
KAGIRLLTWSATAPLDPNDPASPLWGQLVIPEDVLGVKPWADSIHSIRDQNQDVLRDMILAYARSLPATSWLREESRHMHNWRAPS
KYVRLHRQWRDQRIHGDQPAFAALEAWSKQDVHLLHREVPSLERMQRQVEGRVTAVAVQLAKRYGVVARDDFDVTGLVEKDDQHDE
DEQKLRKKTARRVNILGPGRARARVGYFAKKYGSVDLEVDAAGGTLDCAACGHHRDVSREDRVQRVIRCENCGHAEDQDITMSRNL
LAAASAVARGESGWPLDPKVPAPSTKKKAPARRNRRKDQQPLAPSPATP (SEQ ID NO: 28)
>3300031247|Ga0265340_10001032_18|P
[plants-rhizosphere-carex aquatilis]
MTMRVYKFLCLPATAADDHCLREQVRLGARYRRQIALIENRERFLRRALIDYPARDVPVEARGAWWKSEAGKAARDAWHASDEFKA
LRERVFAGKKRALKAVRDATIEAGAMWGTLGKADDAADFARRTIDKAGLIDENGRTTFVGTDFPPDVGRIAVQFQRQNARIDARTG
KTLPAKKSVYADDLIGGEGSWLRIGSVAYSLGAPVDHFRPAAVDAEGRAIDVAGNPGDVIKIGPRAGEAATKGKRFHALAIRVGTV
PGTIRPIWAQMHLLLHQSPRTKNRLQLGHDVVKWAVVRRERTGLRYRWYLTLEVEGSVAVTTPHPHPHDAVAVHIGWRQLFDEEGK
KAGIRLLTWSATAPLDPNDPASPLWGQLVIPEDVLGVKPWADSIHSIRDQNQDVLRDMILAYARSLPATSWLREESRHMHNWRAPS
KYVRLHRQWRDQRIHGDQPAFAALEAWSKQDVHLLHREVPSLERMQRQVEGRVTAVAVQLAKRYGVVARDDFDVTGLVEKDDQHDE
DEQKLRKKTARRVNILGPGRARARVGYFAKKYGSVDLEVDAAGGTLDCAACGHHRDVSREDRVQRVIRCENCGHAEDQDITMSRNL
LAAASAVARGESGWPLDPKVPAPSTKKKAPARRNRRKDQQPLAPSPATP (SEQ ID NO: 29)
>3300031250 Ga0265331_10001842_6|P
[plants-rhizosphere-carex aquatilis]
MTMRVYKFLCLPATAADDHCLREQVRLGARYRRQIALIENRERFLRRALIDYPARDVPVEARGAWWKSEAGKAARDAWHASDEFKA
LRERVFAGKKRALKAVRDATIEAGAMWGTLGKADDAADFARRTIDKAGLIDENGRTTFVGTDFPPDVGRIAVQFQRQNARIDARTG
KTLPAKKSVYADDLIGGEGSWLRIGSVAYSLGAPVDHFRPAAVDAEGRAIDVAGNPGDVIKIGPRAGEAATKGKRFHALAIRVGTV
PGTIRPIWAQMHLLLHQSPRTKNRLQLGHDVVKWAVVRRERTGLRYRWYLTLEVEGSVAVTTPHPHPHDAVAVHIGWRQLFDEEGK
KAGIRLLTWSATAPLDPNDPASPLWGQLVIPEDVLGVKPWADSIHSIRDQNQDVLRDMILAYARSLPATSWLREESRHMHNWRAPS
KYVRLHRQWRDQRIHGDQPAFAALEAWSKQDVHLLHREVPSLERMQRQVEGRVTAVAVQLAKRYGVVARDDFDVTGLVEKDDQHDE
DEQKLRKKTARRVNILGPGRARARVGYFAKKYGSVDLEVDAAGGTLDCAACGHHRDVSREDRVQRVIRCENCGHAEDQDITMSRNL
LAAASAVARGESGWPLDPKVPAPSTKKKAPARRNRRKDQQPLAPSPATP (SEQ ID NO: 30)
>3300031344 Ga0265316_10000001557|P
[plants-rhizosphere-carex aquatilis]
MTMRVYKFLCLPATAADDHCLREQVRLGARYRRQIALIENRERFLRRALIDYPARDVPVEARGAWWKSEAGKAARDAWHASDEFKA
LRERVFAGKKRALKAVRDATIEAGAMWGTLGKADDAADFARRTIDKAGLIDENGRTTFVGTDFPPDVGRIAVQFQRQNARIDARTG
KTLPAKKSVYADDLIGGEGSWLRIGSVAYSLGAPVDHFRPAAVDAEGRAIDVAGNPGDVIKIGPRAGEAATKGKRFHALAIRVGTV
PGTIRPIWAQMHLLLHQSPRTKNRLQLGHDVVKWAVVRRERTGLRYRWYLTLEVEGSVAVTTPHPHPHDAVAVHIGWRQLFDEEGK
KAGIRLLTWSATAPLDPNDPASPLWGQLVIPEDVLGVKPWADSIHSIRDQNQDVLRDMILAYARSLPATSWLREESRHMHNWRAPS
KYVRLHRQWRDQRIHGDQPAFAALEAWSKQDVHLLHREVPSLERMQRQVEGRVTAVAVQLAKRYGVVARDDFDVTGLVEKDDQHDE
DEQKLRKKTARRVNILGPGRARARVGYFAKKYGSVDLEVDAAGGTLDCAACGHHRDVSREDRVQRVIRCENCGHAEDQDITMSRNL
LAAASAVARGESGWPLDPKVPAPSTKKKAPARRNRRKDQQPLAPSPATP (SEQ ID NO: 31)
>3300031733|Ga0316577_10033675_3|M
[plants-rhizosphere-spartina alterniflora]
MPTLVYKYGARSSQDSALLDQMRLGRRYYNALVEAENDRRRAAWGGDHPPAPDDPHETCVCDECVQEPNPEHTSCRCKTCKAHWKA
VRQRVLDTPPLDVKPLYAEARAQGLHWGTCLAIGQAFSAAWTKTSALRTVRFRSWRQGDVAAVQIQLTNKPDSLVRLCKAPDARTG
RRARNGLGRHTVQIRIGSNADRSPIFCDPIRIEKHRELSGRIAWVHVRRKRIADREVWSVAFTCNEADARTDEAHEGVVAVDVSWR
KLPDGSWRLGYAQDDAGNVSELLLPPEWAERAERAERIRSHRDARLNELKAKMPLLAHTKSCRKAVEKLLRLGVLPLELSEWIARD
THLWQYETGCWRRSASRRRAALQEWVRMLRQTYAIVIVKDSQHKLMKEKHDLPQPAQRQGQHAAPGETVENLRRVFGMTGMKVVSA
VDTTTTCVQCGHVTPINGQRFVHCERCGDRRDRDHASTRNMLQLYKNGAYKSPTARKTTSRFAKKHTKKDVPPEC (SEQ ID
NO: 32)
>SRR8543844_2229942_334|P
[rhizosphere metagenome]
MTMRVYKFLCLPATAADDHCLREQVRLGARYRRQIALIENRERFLRRALIDYPARDVPVEARGAWWKSEAGKAARDAWHASDEFKA
LRERVFAGKKRALKAVRDATIEAGAMWGTLGKADDAADFARRTIDKAGLIDENGRTTFVGTDFPPDVGRIAVQFQRQNARIDARTG
KTLPAKKSVYADDLIGGEGSWLRIGSVAYSLGAPVDHFRPAAVDAEGRAIDVAGNPGDVIKIGPRAGEAATKGKRFHALAIRVGTV
PGTIRPIWAQMHLLLHQSPRTKNRLQLGHDVVKWAVVRRERTGLRYRWYLTLEVEGSVAVTTPHPHPHDAVAVHIGWRQLFDEEGK
KAGIRLLTWSATAPLDPNDPASPLWGQLVIPEDVLGVKPWADSIHSIRDQNQDVLRDMILAYARSLPATSWLREESRHMHNWRAPS
KYVRLHRQWRDQRIHGDQPAFAALEAWSKQDVHLLHREVPSLERMQRQVEGRVTAVAVQLAKRYGVVARDDFDVTGLVEKDDQHDE
DEQKLRKKTARRVNILGPGRARARVGYFAKKYGSVDLEVDAAGGTLDCAACGHHRDVSREDRVQRVIRCENCGHAEDQDITMSRNL
LAAASAVARGESGWPLDPKVPAPSTKKKAPARRNRRKDQQPLAPSPATP (SEQ ID NO: 33)
>SRR8543847_1991039_375|P
[rhizosphere metagenome]
MTMRVYKFLCLPATAADDHCLREQVRLGARYRRQIALIENRERFLRRALIDYPARDVPVEARGAWWKSEAGKAARDAWHASDEFKA
LRERVFAGKKRALKAVRDATIEAGAMWGTLGKADDAADFARRTIDKAGLIDENGRTTFVGTDFPPDVGRIAVQFQRQNARIDARTG
KTLPAKKSVYADDLIGGEGSWLRIGSVAYSLGAPVDHFRPAAVDAEGRAIDVAGNPGDVIKIGPRAGEAATKGKRFHALAIRVGTV
PGTIRPIWAQMHLLLHQSPRTKNRLQLGHDVVKWAVVRRERTGLRYRWYLTLEVEGSVAVTTPHPHPHDAVAVHIGWRQLFDEEGK
KAGIRLLTWSATAPLDPNDPASPLWGQLVIPEDVLGVKPWADSIHSIRDQNQDVLRDMILAYARSLPATSWLREESRHMHNWRAPS
KYVRLHRQWRDQRIHGDQPAFAALEAWSKQDVHLLHREVPSLERMQRQVEGRVTAVAVQLAKRYGVVARDDFDVTGLVEKDDQHDE
DEQKLRKKTARRVNILGPGRARARVGYFAKKYGSVDLEVDAAGGTLDCAACGHHRDVSREDRVQRVIRCENCGHAEDQDITMSRNL
LAAASAVARGESGWPLDPKVPAPSTKKKAPARRNRRKDQQPLAPSPATP (SEQ ID NO: 34)
>SRR8543989_2137428_34|M
[rhizosphere metagenome]
MTMRVYKFLCLPATAADDHCLREQVRLGARYRRQIALIENRERFLRRALIDYPARDVPVEARGAWWKSEAGKAARDAWHASDEFKA
LRERVFAGKKRALKAVRDATIEAGAMWGTLGKADDAADFARRTIDKAGLIDENGRTTFVGTDFPPDVGRIAVQFQRQNARIDARTG
KTLPAKKSVYADDLIGGEGSWLRIGSVAYSLGAPVDHFRPAAVDAEGRAIDVAGNPGDVIKIGPRAGEAATKGKRFHALAIRVGTV
PGTIRPIWAQMHLLLHQSPRTKNRLQLGHDVVKWAVVRRERTGLRYRWYLTLEVEGSVAVTTPHPHPHDAVAVHIGWRQLFDEEGK
KAGIRLLTWSATAPLDPNDPASPLWGQLVIPEDVLGVKPWADSIHSIRDQNQDVLRDMILAYARSLPATSWLREESRHMHNWRAPS
KYVRLHRQWRDQRIHGDQPAFAALEAWSKQDVHLLHREVPSLERMQRQVEGRVTAVAVQLAKRYGVVARDDFDVTGLVEKDDQHDE
DEQKLRKKTARRVNILGPGRARARVGYFAKKYGSVDLEVDAAGGTLDCAACGHHRDVSREDRVQRVIRCENCGHAEDQDITMSRNL
LAAASAVARGESGWPLDPKVPAPSTKKKAPARRNRRKDQQPLAPSPATP (SEQ ID NO: 35)
>SRR8543995_2079833_6|M
[rhizosphere metagenome]
MTMRVYKFLCLPATAADDHCLREQVRLGARYRRQIALIENRERFLRRALIDYPARDVPVEARGAWWKSEAGKAARDAWHASDEFKA
LRERVFAGKKRALKAVRDATIEAGAMWGTLGKADDAADFARRTIDKAGLIDENGRTTFVGTDFPPDVGRIAVQFQRQNARIDARTG
KTLPAKKSVYADDLIGGEGSWLRIGSVAYSLGAPVDHFRPAAVDAEGRAIDVAGNPGDVIKIGPRAGEAATKGKRFHALAIRVGTV
PGTIRPIWAQMHLLLHQSPRTKNRLQLGHDVVKWAVVRRERTGLRYRWYLTLEVEGSVAVTTPHPHPHDAVAVHIGWRQLFDEEGK
KAGIRLLTWSATAPLDPNDPASPLWGQLVIPEDVLGVKPWADSIHSIRDQNQDVLRDMILAYARSLPATSWLREESRHMHNWRAPS
KYVRLHRQWRDQRIHGDQPAFAALEAWSKQDVHLLHREVPSLERMQRQVEGRVTAVAVQLAKRYGVVARDDFDVTGLVEKDDQHDE
DEQKLRKKTARRVNILGPGRARARVGYFAKKYGSVDLEVDAAGGTLDCAACGHHRDVSREDRVQRVIRCENCGHAEDQDITMSRNL
LAAASAVARGESGWPLDPKVPAPSTKKKAPARRNRRKDQQPLAPSPATP (SEQ ID NO: 36)
>SRR8544146_618080_5|P
[rhizosphere metagenome]
MTMRVYKFLCLPATAADDHCLREQVRLGARYRRQIALIENRERFLRRALIDYPARDVPVEARGAWWKSEAGKAARDAWHASDEFKA
LRERVFAGKKRALKAVRDATIEAGAMWGTLGKADDAADFARRTIDKAGLIDENGRTTFVGTDFPPDVGRIAVQFQRQNARIDARTG
KTLPAKKSVYADDLIGGEGSWLRIGSVAYSLGAPVDHFRPAAVDAEGRAIDVAGNPGDVIKIGPRAGEAATKGKRFHALAIRVGTV
PGTIRPIWAQMHLLLHQSPRTKNRLQLGHDVVKWAVVRRERTGLRYRWYLTLEVEGSVAVTTPHPHPHDAVAVHIGWRQLFDEEGK
KAGIRLLTWSATAPLDPNDPASPLWGQLVIPEDVLGVKPWADSIHSIRDQNQDVLRDMILAYARSLPATSWLREESRHMHNWRAPS
KYVRLHRQWRDQRIHGDQPAFAALEAWSKQDVHLLHREVPSLERMQRQVEGRVTAVAVQLAKRYGVVARDDFDVTGLVEKDDQHDE
DEQKLRKKTARRVNILGPGRARARVGYFAKKYGSVDLEVDAAGGTLDCAACGHHRDVSREDRVQRVIRCENCGHAEDQDITMSRNL
LAAASAVARGESGWPLDPKVPAPSTKKKAPARRNRRKDQQPLAPSPATP (SEQ ID NO: 37)
>SRR6963591_1323413_2|M
[sediment metagenome]
MKVFKYGARPVNQESLIRDQMKLARNYYNKLVETENKRRQQAWGSEKPPPPPHPPEEVVLPDGSKKTKWCRCEECKTHWKTLREAY
YALDNLDKKPLRAAASSWGLYWGSYLLVEQAFDQAQKTTVYYDPVRFRGWRKGGQAGLQIQNGKNTDSLFKVEEIPDPEPSARSKK
NDRRAGKGKRRGQLRTLRIRIGTEGRTPIWSDPIIFKMHRPLEGTVCWLKICMFYRGDREIWSVNFTCKDTSPRTDSATDGVVAID
VSWRQLPNGDIRLAYARGDNGFEDELTLDNSWLERIKKAERIQSHRDERLNDLKTSDSRFRLFKSPRSARIHIMKNQIAELEDWAK
REKHLEQYELGCKRKTYDVRRDAVRVWLRKLRRLYQTVVIKDSSHKEMKEQAKAKKKLHRAARKQGHHGAPGEIVEEICKVFGRIE
NVAVVIAENTTATCPVCGHMHEVNKERMITCERCGTTDDRDRISTHNLLSRFFDGQYEKPTARKTESKFAKKHKKVEQEVKHLRDG
L (SEQ ID NO: 38)
>SRR7976138_3273187_71|P
[soi1|Metagenome]
MMCFQYWAEPVDETSKALLAHQLREGAMYRRILATLENRARFLLGGLMRLTKEDRAPWRARIYVAHAQAVRDERAAASARGLTWGT
YLAIEDALDQSQRTKARGAMLGTFAPRDEGIVGVHIQNREISARAVVGGEDKYVRIDPELVGKPTLRSRREGTAMGARRLNILSIR
VGSKGPRGLEPVWARFYVLMNRPLPDASLKWVKLRCRRIGTTYRWQANFVVGAPCVGTPNPDRLPGVGIDLGWRKRADGIRIAYYA
DTEGRHGELVIPQHVIARDAKSDSLRRIRDQARNQIQAVLADLRTRLASRKAVSSIPPVEGTAVTPAWWRWTCGLAVGMALLTPTE
AWFLEQTQFLHAWRKVSRLVWLHRQWRERRFAGDEDAFLLMDDYIRQDRHLFNWEAYNRRRLTAEVEGRVREFAVSLAKRYDMIAV
EEAGIVSKLVQKQAEPEPRDEALRAVAAKRMHACSPASVREQIVRFGGKYGAKVMQIDPANTTKTCDACGQIRAPADPEQLVLGCE
HCGHVQDQDHSAARTLLASGLVMALEGAATKGRTPRKMGARRTRKKAQVEIARDA (SEQ ID NO: 39)
>SRR7976138_3273187_70|M
[soi1|Metagenome]
MAVADPEGIEIMMCFQYWAEPVDETSKALLAHQLREGAMYRRILATLENRARFLLGGLMRLTKEDRAPWRARIYVAHAQAVRDERA
AASARGLTWGTYLAIEDALDQSQRTKARGAMLGTFAPRDEGIVGVHIQNREISARAVVGGEDKYVRIDPELVGKPTLRSRREGTAM
GARRLNILSIRVGSKGPRGLEPVWARFYVLMNRPLPDASLKWVKLRCRRIGTTYRWQANFVVGAPCVGTPNPDRLPGVGIDLGWRK
RADGIRIAYYADTEGRHGELVIPQHVIARDAKSDSLRRIRDQARNQIQAVLADLRTRLASRKAVSSIPPVEGTAVTPAWWRWTCGL
AVGMALLTPTEAWFLEQTQFLHAWRKVSRLVWLHRQWRERRFAGDEDAFLLMDDYIRQDRHLFNWEAYNRRRLTAEVEGRVREFAV
SLAKRYDMIAVEEAGIVSKLVQKQAEPEPRDEALRAVAAKRMHACSPASVREQIVRFGGKYGAKVMQIDPANTTKTCDACGQIRAP
ADPEQLVLGCEHCGHVQDQDHSAARTLLASGLVMALEGAATKGRTPRKMGARRTRKKAQVEIARDA (SEQ ID NO: 40)
>3300012943|Ga0164241_10000441_40|P
[terrestrial-soil]
MMCFQYWAEPVDETSKALLAHQLREGAMYRRILATLENRARFLLGGLMRLTKEDRAPWRARIYVAHAQAVRDERAAASARGLTWGT
YLAIEDALDQSQRTKARGAMLGTFAPRDEGIVGVHIQNREISARAVVGGEDKYVRIDPELVGKPTLRSRREGTAMGARRLNILSIR
VGSKGPRGLEPVWARFYVLMNRPLPDASLKWVKLRCRRIGTTYRWQANFVVGAPCVGTPNPDRLPGVGIDLGWRKRADGIRIAYYA
DTEGRHGELVIPQHVIARDAKSDSLRRIRDQARNQIQAVLADLRTRLASRKAVSSIPPVEGTAVTPAWWRWTCGLAVGMALLTPTE
AWFLEQTQFLHAWRKVSRLVWLHRQWRERRFAGDEDAFLLMDDYIRQDRHLFNWEAYNRRRLTAEVEGRVREFAVSLAKRYDMIAV
EEAGIVSKLVQKQAEPEPRDEALRAVAAKRMHACSPASVREQIVRFGGKYGAKVMQIDPANTTKTCDACGQIRAPADPEQLVLGCE
HCGHVQDQDHSAARTLLASGLVMALEGAATKGRTPRKMGARRTRKKAQVEIARDA (SEQ ID NO: 41)
>3300012943|Ga0164241_10000441_39|M
[terrestrial-soil]
MAVADPEGIEIMMCFQYWAEPVDETSKALLAHQLREGAMYRRILATLENRARFLLGGLMRLTKEDRAPWRARIYVAHAQAVRDERA
AASARGLTWGTYLAIEDALDQSQRTKARGAMLGTFAPRDEGIVGVHIQNREISARAVVGGEDKYVRIDPELVGKPTLRSRREGTAM
GARRLNILSIRVGSKGPRGLEPVWARFYVLMNRPLPDASLKWVKLRCRRIGTTYRWQANFVVGAPCVGTPNPDRLPGVGIDLGWRK
RADGIRIAYYADTEGRHGELVIPQHVIARDAKSDSLRRIRDQARNQIQAVLADLRTRLASRKAVSSIPPVEGTAVTPAWWRWTCGL
AVGMALLTPTEAWFLEQTQFLHAWRKVSRLVWLHRQWRERRFAGDEDAFLLMDDYIRQDRHLFNWEAYNRRRLTAEVEGRVREFAV
SLAKRYDMIAVEEAGIVSKLVQKQAEPEPRDEALRAVAAKRMHACSPASVREQIVRFGGKYGAKVMQIDPANTTKTCDACGQIRAP
ADPEQLVLGCEHCGHVQDQDHSAARTLLASGLVMALEGAATKGRTPRKMGARRTRKKAQVEIARDA (SEQ ID NO: 42)
>3300031949|Ga0214473_10000008_352|M
[terrestrial-soil]
MRVYKFLCIPATAADDRALRDQVYLGSRYRRQSALIENRARVLQRGIADRPALNVPEKDRSAWWKSEEGKAARDAWYASDECKMLR
EIIFAEKKRALKAARDAAIEAGTMWGTCGKADDAADFARRAVDKNGLIDEDGQTTFVDTHFPADVGRIAVQFQRGNERIDTRTGKK
IPAKKLIYADDLIGDEDTRLRIGTVAYALAEPVDGFRPAAIDAEGKAIDVYTRGLSEREQDLLARDQREEAIAAYAARTPASIDPS
GNICKTPEQKAVTKIVRWERGERDEKGKRYHSLAIRVGTMPGTIRPIWAHLHVMLHQSPRSKNQLVLGHDVVKWAVIRRERTGLRY
RWFLTLETEGDVAVTSPHPQPHDAIAVHIGWRQLFSEAGDPAGIRVLTWSASAPLDPDDPRSPREGQLVLPQEVVDQKPFADRIHS
TRDKNQDVLRDMILVYARSLPTSSWLRVEAAHMHQWRAPRKYVRLYCQWKEQRIHGDQAAFAALEAWIKQDRHLLHREAPSIERMK
RQIEGRVTALAVQLAKRYGVVASDNIAVGELVKKDDRHDEDEQKLRKKTARRVNVVAPGEARALVRYFAKKYGCVGLELDAAGGTI
DCDACGHRRDVAREDRVQRMIRCENCGHVEDQDLTMSRNLLSGASAVARGESGWPLAPKVPAASTKKRATVRRNRRRGQQPLASSV
GTP (SEQ ID NO: 43)
>3300031965|Ga0326597_10001868_12|M
[terrestrial-soil]
MIEDAFAMACKDRSVLRLVKFRSWRDGGVAAVQIQRAAWERDGGASMYRTRAQVDNRTGRRTKHGGRQTLEIRIGSDGRNPIWSEP
VRYEQHREAVGRPTWIKVSMKYRGDLEVWSVVVTCADVPPRPASEEAQRGVVAVDVGFRRVGDDIRIAYARDDRGKVSELLMDARW
RELCNRADRIRGHRDDNFNALQEADSENLFSLTKSANGARQKIERLVREGEEVSQEMRAWMHRDRHLSQYEDGCRRRSVNARKDAM
RKWARELRRNYSQLVLSKTAVKKMKENAKDDGLPKPARRQGQNAAPGELLDWLVMVFGRAEVAIVKPIHTTDTCVSCEHVNDHGPE
TIVVCEECGAALDRDEASTRNMMDLWVNGECEKPTARKASAKFAKKHKTEDKDDNASPAA (SEQ ID NO: 44)
>3300006795|Ga0075520_1037237_1|M
[terrestrial-soil-arctic peat soil]
MAFVRETILPEIDAFNDALGRSKAKIKHPAKLKTEMPGIDGLWHFVGQLRTRIEKGRAVPEGLLEKIIAFAEQFKPDYTLLNEFLN
NYRDIAAKEATALGLLRYEIRPTVNGFKAVLDRRKTTKAAWSEGWPLIKYPDSPKAANWGLHYYFNMAGLDSAQIESPKGVPGLTF
GPHLKSSDTGHELLTGLAATKRSLRAAEISISGSNHERCTFRFAVLYHRPLPAGSHLKEWKLIYADGALWLCLTVELQHPVPVHSP
LAAGLDVGWRRTEEGLRFGTLYEPATMSFRELSIDMQKSPKDHKDRMPFRIDIGPTRWEKRNILQLLPDWKPGNPIPSSFETRTAV
QSRRDYYKDTAKILLRKHLGESMPAWIDKAGRRGLLKLAEEFKEDQVVQNILGTWMQQDEQVGKLVSMYLARSTKRLEYGHAQVAH
DVCRHLLDKGVHRLIVETSFLAKVSQRHDNEDSEGLKRSQKYRQFAAVGKFVAVLKNTAIKYGIVVEELSTINTTRICQYCNHLNP
GTGKEYFTCEKCERQIKQDHNAAVNISRFGCDPELAEMAASAG (SEQ ID NO: 45)
>3300014491|Ga0182014_10039677_1|P
[terrestrial-soil-bog]
MIDRLTFDGKCVYVHLAAISLKATKVFTHGGWVFDLAAHTVAFKDIPPVALPENLTLSDGASLTFQLVAPDGTLRKQVRSGVLKSN
DKQPGILRIAGRSKTPANFSAADGWKIEVFKYRAYFTHPGLKTEAGLPEWLQNSIKRQRDFWNRMAWLCRDARRRCSPVPTEEIVA
FVQESVLPAIDEFNNSLGIARSKEKIKHPINLKIEMPGLDAVWKFVGELRKRIAKNRGVPDGLLEKVVAFAEQFKADYGPLNEFIS
NLESIAKIESTALGLRQFEIRPTINSFKAVLDRRKTLKSAWSDGWPHIKYADSPRAADWGVYYYFNKSGVDSARLEAGPGVPGLTF
GRPLKPADTGHKSMKSPKRSSRALREAKISIAGDNREEWAFKFGVLQHRPLPPDSHLKQWSLIYSGGALWLCFTVERKRPLPFPGM
HAAGLDIGWRRTEEGIRFGILYEPESKTFRELSIDLQKSPEDHNNRTPFRIDMGPNRWDKRNITQLLPDWKPGDAIPNTVVVRRAL
GSRRSSYKDAAKVLLQKHLGDQLPVWFDKAGSRGLFKLKENFKDVPVVQEIIDDLQKKTEALNAVAGKYTARYTRELEYGQIQIAH
DVCRHLQQKGVTRLIVEASFVAKASQKQDNEDPASLKNSQKYRQFAAVSKFVSLLKNIAVKYGIAVEVLSAQNTTRICQYCNHLNP
STEKEIFFCEGCGKQVQQDQNAAVNLSRFGIDPELAEMALTFSKD (SEQ ID NO: 46)
>3300014491|Ga0182014_10071250_1|M
[terrestrial-soil-bog]
MAFVRETILPEIDAFNDALGRSKAKIKHPAKLKTEMPGPDGLWNFVGQLRTRIEKGRAVPEGLLEKIIAFAEQFKPDYTPLNEFLN
KYHNIAAKEATDLGLLRYEIRPTVSGFKAVLDRRKTTKAAWSEGWPLIKYPDSPKAANWGVHYYFNTAGLDSAQIESPKGVPGLTL
GPHLKPSDTGHELLTGLAATKRSLRAAEISISGPNHEQCTFRFGVLYHRPLPAGSHLKEWKLIYADGALWLCLTVELQHPVPVHSP
LAAGLDVGWRRTEEGLRFGTLYEPATMSFRELSIDMQKSPKDHKDRMPFRIDIGPTRWEKRNILQLLPDWKPGNPIPSSFETRTAM
QSRRDYYKDTAKILLRKHLGESMPAWIDKAGRRGLLKLVEEFKEDEVVQNILGTWKQQDEEVGKLVSMYLARSTKRLEYGQAQVAH
DVCRYLHDKGVHRLIVETSFLAKVSQRHDNEDSEGLKRSQKYRQFAAVGKFVAVLKNTAIKYGIVVEELSAINTTRICQYCNHLNP
GTEKEHFTCEKCERQIKQDHNAAVNISRFGCDPELAEMAASAG (SEQ ID NO: 47)
>3300017975Ga0187782_10004712_9|P
[terrestrial-soil-tropical peatland]
MATKVLKFYCVPASARDNQLLCEQVKLGSMYRRDLAWIENRARELRRAIADWPARDVPIEKRSEWWKSDVGRAASKAWHESDECKR
LKKSITDGQIQAATAARKKARRFDTAWGTCGLAAEAAEAARVALEKEFGLIDKETGQTTFVNTKFPSDEGRVGVQFQRDTDKIDEE
TGEVIPAKKPTIYADELIGGSHNFCRIGSARYSLLDRVDGFQPAALDASGAPIDSFGYKSKQPKGKRFHLLQIRVGTVDKRSTAYP
FDLDPDKGEPIWAKLHVLSHQKTKRPQTLPHERVKWVFVQRVRCALRYRWSVCFVIEDSVVQKPHEQPNDRVAVDLGWRQLFDESG
TPAGIRLLYWMATSPLDLNDPSSPVEGQLVIPQHVVDRKPFSSQLESNRTKNREAMQAMLLAYLQSVSSATWLAKRTAQLASWKKP
SKFVLLLNEWRKNRIAGDQAAFAALEAWNKQDAHLYLWSAPNITKMQRQIQGRVDQFAVQLARRYGIVIVENFKLPDVIEKKDLQE
QDQDAQKLRKKNARRVNVVAPGRARAALKRFATKYNSHYIEEESAFTTVDCASCGHRREFDLKNARAQLLLACDNCGVVEDQDRTA
ARNLLAGASAIARGENGWPLEAKVSKASKKKVVLRRTRKRIVV (SEQ ID NO: 48)
>3300017975|Ga0187782_10004712_9|M
[terrestrial-soil-tropical peatland]
MYRRDLAWIENRARELRRAIADWPARDVPIEKRSEWWKSDVGRAASKAWHESDECKRLKKSITDGQIQAATAARKKARRFDTAWGT
CGLAAEAAEAARVALEKEFGLIDKETGQTTFVNTKFPSDEGRVGVQFQRDTDKIDEETGEVIPAKKPTIYADELIGGSHNFCRIGS
ARYSLLDRVDGFQPAALDASGAPIDSFGYKSKQPKGKRFHLLQIRVGTVDKRSTAYPFDLDPDKGEPIWAKLHVLSHQKTKRPQTL
PHERVKWVFVQRVRCALRYRWSVCFVIEDSVVQKPHEQPNDRVAVDLGWRQLFDESGTPAGIRLLYWMATSPLDLNDPSSPVEGQL
VIPQHVVDRKPFSSQLESNRTKNREAMQAMLLAYLQSVSSATWLAKRTAQLASWKKPSKFVLLLNEWRKNRIAGDQAAFAALEAWN
KQDAHLYLWSAPNITKMQRQIQGRVDQFAVQLARRYGIVIVENFKLPDVIEKKDLQEQDQDAQKLRKKNARRVNVVAPGRARAALK
RFATKYNSHYIEEESAFTTVDCASCGHRREFDLKNARAQLLLACDNCGVVEDQDRTAARNLLAGASAIARGENGWPLEAKVSKASK
KKVVLRRTRKRIVV (SEQ ID NO: 49)
>SRR7027835_762984_1|M
[wetland metagenome]
MAWLCRETRLKCSPVPTDKIVDFVQNAILPEIDAFNSAPGRSRERMKHPAKLKIETPGLDCLWGFVGELRARTGKGRAVPAGLLDK
VVEFAQQFKTDYTPFNEFIAGFQAIAEREAAALGLRHFEIRPTVGAFKAVLDRRKTTKAHWSEGWPLIKYPDSPKAANWGLHYHLN
KAGVDSALLEREEGIPGLSFGPRLKPADTGHEKLTGVAAKRILREAQISISGDNDERWTFHFGVLQHRPLPPNSHLKEWKLIFQGG
ALWLCLVVELQRPVPVPGPHPAGLDIGWRRTEEGIRFGTLYEPATKTIRELTVDLQKSPCDHKDRVPFRIDLGPSRWEMRNATELV
PDWKPGQPIPSAFETRSVLQIRRDRIKETAKRQLREHLGERLPVWFDKAGRRGLLKLAQDFKNESAVCEILNIWQQKDEQLGKLAS
MYFDRSTKRIEYGHAQVAHDVCRFLQQKGVMRLIVETSFLSKVSQHQDNEDLVSIERSQKYRQFVAVGKFLAALKNTTRKYGIVVE
TVNAMNTTRTCQYCNALNPRTEEEQFTCKRCGRQVQQDHNAAVNLSRFGSDPVLAEMALHADEV (SEQ ID NO: 50)

TABLE 4
Nucleotide sequences of Representative CLUST. 133120 Direct Repeats

| CLUST.133120 Effector Protein | | Spacer |
|---|---|---|
| Accession | Direct Repeat Nucleotide Sequence | Length(s) |
| 3300027740\|Ga0214474_1000103_171 | CCAACCAATGCCAGCGCGACGGCTTATGAGTCG | 35-36 |
| M (SEQ ID NO: 1) | CGAC (SEQ ID NO: 51) | |
| | GTCGCGACTCATAAGCCGTCGCGCTGGCATTGG | |
| | TTGG (SEQ ID NO: 95) | |
| 3300017971 \|Ga018043 8_10084474_2\| | GGTAAAACACCTGCGAGATGGTTTATGAATCTC | 36-41 |
| M (SEQ ID NO: 2) | GAC (SEQ ID NO: 52) | |
| | GTCGAGATTCATAAACCATCTCGCAGGTGTTTT | |
| | ACC (SEQ ID NO: 96) | |
| 3300022592\|Ga0236342_1001376_7\|P | AGTGCATCGAACAGATGCAAC (SEQ ID NO: | 24-51 |
| (SEQ ID NO: 3) | 53) | |
| | GTTGCATCTGTTCGATGCACT (SEQ ID NO: | |
| | 97) | |
| 3300020171\|Ga0180732_1000630_46\| | AATGCCAGCCCAACGGCCTAGAAGTTGGGAC | 26-52 |
| P (SEQ ID NO: 4) | (SEQ ID NO: 54) | |
| | GTCCCAACTTCTAGGCCGTTGGGCTGGCATT | |
| | (SEQ ID NO: 98) | |
| 3300018040\|Ga0187862_10024620_1\| | GTAACAGTGGCATCACAGTGCATTGAATAGATG | 34-46 |
| M (SEQ ID NO: 5) | CAAC (SEQ ID NO: 55) | |
| | GTTGCATCTATTCAATGCACTGTGATGCCACTG | |
| | TTAC (SEQ ID NO: 99) | |
| 3300005253\|Ga0073583_1150562_58\| | GTAGCAATGGCAGCGCATCGCCTTTTAAGATGC | 24-39 |
| P (SEQ ID NO: 6) | GAC (SEQ ID NO: 56) | |
| | GTCGCATCTTAAAAGGCGATGCGCTGCCATTGC | |
| | TAC (SEQ ID NO: 100) | |
| 3300005253\|Ga0073583_1150562_51\| | GTAGCAATGGCAGCGCATCGCCTTTTAAGATGC | 24-39 |
| M (SEQ ID NO: 7) | GAC (SEQ ID NO: 56) | |
| | GTCGCATCTTAAAAGGCGATGCGCTGCCATTGC | |
| | TAC (SEQ ID NO: 100) | |
| LAZR01002400_19\|P (SEQ ID NO: 8) | GTAGCAATGGCAGCGCATCGCCTTTTAAGATGC | 24-39 |
| | GAC (SEQ ID NO: 56) | |
| | GTCGCATCTTAAAAGGCGATGCGCTGCCATTGC | |
| | TAC (SEQ ID NO: 100) | |
| LAZR01002400_15\|M (SEQ ID NO: | GTAGCAATGGCAGCGCATCGCCTTTTAAGATGC | 24-39 |
| 9) | GAC (SEQ ID NO: 56) | |
| | GTCGCATCTTAAAAGGCGATGCGCTGCCATTGC | |
| | TAC (SEQ ID NO: 100) | |
| SDBU01080949_7\|P (SEQ ID NO: 10) | GTAGCAATGGCAGCGCATCGCCTTT (SEQ ID | 49 |
| | NO: 57) | |
| | AAAGGCGATGCGCTGCCATTGCTAC (SEQ ID | |
| | NO: 900) | |
| SDBU01080949_6\|M (SEQ ID NO: | GTAGCAATGGCAGCGCATCGCCTTT (SEQ ID | 49 |
| 11) | NO: 57) | |
| | AAAGGCGATGCGCTGCCATTGCTAC (SEQ ID | |
| | NO: 900) | |
| SDBV01087475_1\|P (SEQ ID NO: 12) | GTAGCAATGGCAGCGCATCGCCTTTTAAGATGC | 35-39 |
| | GAC (SEQ ID NO: 56) | |
| | GTCGCATCTTAAAAGGCGATGCGCTGCCATTGC | |
| | TAC (SEQ ID NO: 100) | |
| SDBV01087475_1\|M (SEQ ID NO: | GTAGCAATGGCAGCGCATCGCCTTTTAAGATGC | 35-39 |
| 13) | GAC (SEQ ID NO: 56) | |
| | GTCGCATCTTAAAAGGCGATGCGCTGCCATTGC | |
| | TAC (SEQ ID NO: 100) | |
| SRR2657585_2135055_9\|M (SEQ ID | GGATCAATGCTTGCTCGATGGCTT (SEQ ID | 30-49 |
| NO: 14) | NO: 58) | |
| | AAGCCATCGAGCAAGCATTGATCC (SEQ ID | |
| | NO: 901) | |
| SRR2657585_2135055_8\|P (SEQ ID | GGATCAATGCTTGCTCGATGGCTT (SEQ ID | 30-49 |
| NO: 15) | NO: 58) | |
| | AAGCCATCGAGCAAGCATTGATCC (SEQ ID | |
| | NO: 901) | |
| SRR2657585_1667169_1\|M (SEQ ID | CCAACAATGCTAGCGAGACGGCTTCATGATCTC | 35-38 |
| NO: 16) | GACG (SEQ ID NO: 59) | |
| | CGTCGAGATCATGAAGCCGTCTCGCTAGCATTG | |
| | TTGG (SEQ ID NO: 902) | |
| SRR8451930_3283449_6\|P (SEQ ID | GTAGCAATGGCAGCGCATCGCCTTTTAAGATGC | 37-51 |
| NO: 17) | GAC (SEQ ID NO: 56) | |
| | GTCGCATCTTAAAAGGCGATGCGCTGCCATTGC | |
| | TAC (SEQ ID NO: 100) | |
| SRR8451930_3283449_6\|M (SEQ ID | GTAGCAATGGCAGCGCATCGCCTTTTAAGATGC | 37-51 |
| NO: 18) | GAC (SEQ ID NO: 56) | |
| | GTCGCATCTTAAAAGGCGATGCGCTGCCATTGC | |
| | TAC (SEQ ID NO: 100) | |
| SRR8452060_665073_11\|P (SEQ ID | GTAGCAATGGCAGCGCATCGCCTTTCAAGATGC | 36-48 |
| NO: 19) | GAC (SEQ ID NO: 60) | |
| | GTCGCATCTTGAAAGGCGATGCGCTGCCATTGC | |
| | TAC (SEQ ID NO: 903) | |
| SRR8452060_665073_10\|M (SEQ ID | GTAGCAATGGCAGCGCATCGCCTTTCAAGATGC | 36-48 |
| NO: 20) | GAC (SEQ ID NO: 60) | |
| | GTCGCATCTTGAAAGGCGATGCGCTGCCATTGC | |
| | TAC (SEQ ID NO: 903) | |
| SRR6448207_2353026_47\|M (SEQ ID | GTCGCAATCTTTGCCGAGTCAAGCATGAACTCG | 36-37 |
| NO: 21) | GACC (SEQ ID NO: 61) | |
| | GGTCCGAGTTCATGCTTGACTCGGCAAAGATTG | |
| | CGAC (SEQ ID NO: 904) | |
| SRR7004342_2720286_9\|P (SEQ ID | GTATTCACGCTTGCGAGGACGGATTGCGTCCTC | 35-46 |
| NO: 22) | GAC (SEQ ID NO: 62) | |
| | GTCGAGGACGCAATCCGTCCTCGCAAGCGTGAA | |
| | TAC (SEQ ID NO: 905) | |
| SRR7004342_2720286_9\|M (SEQ ID | GTATTCACGCTTGCGAGGACGGATTGCGTCCTC | 35-46 |
| NO: 23) | GAC (SEQ ID NO: 62) | |
| | GTCGAGGACGCAATCCGTCCTCGCAAGCGTGAA | |
| | TAC (SEQ ID NO: 905) | |
| SRR6491179_278020_2\|M (SEQ ID | GTACAACGAACTGAAAGTGCGCCGAACAGGTGC | 34-41 |
| NO: 24) | AAC (SEQ ID NO: 63) | |
| | GTTGCACCTGTTCGGCGCACTTTCAGTTCGTTG | |
| | TAC (SEQ ID NO: 906) | |
| 3300028800\|Ga0265338_10000470_3 | GGCACGATCGATGCGAGACCTCGCGGTGGTCTC | 35-54 |
| 8\|P (SEQ ID NO: 25) | GAC (SEQ ID NO: 64) | |
| | GTCGAGACCACCGCGAGGTCTCGCATCGATCGT | |
| | GCC (SEQ ID NO: 907) | |
| 3300031235\|Ga0265330_10003750_4l | GGCACGATCGATGCGAGACCTCGCGGTGGTCTC | 36-50 |
| P (SEQ ID NO: 26) | GAC (SEQ ID NO: 64) | |
| | GTCGAGACCACCGCGAGGTCTCGCATCGATCGT | |
| | GCC (SEQ ID NO: 907) | |
| 3300031241 \|Ga0265325_10000012_1 | GGCACGATCGATGCGAGACCTCGCGGTGGTCTC | 35-54 |
| 20\|M (SEQ ID NO: 27) | GAC (SEQ ID NO: 64) | |
| | GTCGAGACCACCGCGAGGTCTCGCATCGATCGT | |
| | GCC (SEQ ID NO: 907) | |
| 3300031242\|Ga0265329_10001065_51 | GGCACGATCGATGCGAGACCTCGCGGTGGTCTC | 35-50 |
| P (SEQ ID NO: 28) | GAC (SEQ ID NO: 64) | |
| | GTCGAGACCACCGCGAGGTCTCGCATCGATCGT | |
| | GCC (SEQ ID NO: 907) | |
| 3300031247\|Ga0265340_10001032_1 | GGCACGATCGATGCGAGACCTCGCGGTGGTCTC | 36-49 |
| 8\|P (SEQ ID NO: 29) | GAC (SEQ ID NO: 64) | |
| | GTCGAGACCACCGCGAGGTCTCGCATCGATCGT | |
| | GCC (SEQ ID NO: 907) | |
| 3300031250\|Ga0265331_10001842_6\| | GGCACGATCGATGCGAGACCTCGCGGTGGTCTC | 36-49 |
| P (SEQ ID NO: 30) | GAC (SEQ ID NO: 64) | |
| | GTCGAGACCACCGCGAGGTCTCGCATCGATCGT | |
| | GCC (SEQ ID NO: 907) | |
| 3300031344\|Ga0265316_10000001_5 | GGCACGATCGATGCGAGACCTCGCGGTGGTCTC | 35-54 |
| 57\|P (SEQ ID NO: 31) | GAC (SEQ ID NO: 64) | |
| | GTCGAGACCACCGCGAGGTCTCGCATCGATCGT | |
| | GCC (SEQ ID NO: 907) | |
| 3300031733\|Ga0316577_10033675_3\| | CCCGGAATGCTAGCTCGATGGCTATTCAATCGA | 19-38 |
| M (SEQ ID NO: 32) | GAC (SEQ ID NO: 65) | |
| | GTCTCGATTGAATAGCCATCGAGCTAGCATTCC | |
| | GGG (SEQ ID NO: 908) | |
| SRR8543844_2229942_334\|P (SEQ | GGCACGATCGATGCGAGACCTCGCGGTGGTCTC | 35-54 |
| ID NO: 33) | GAC (SEQ ID NO: 64) | |
| | GTCGAGACCACCGCGAGGTCTCGCATCGATCGT | |
| | GCC (SEQ ID NO: 907) | |
| SRR8543847_1991039_375\|P (SEQ | GGCACGATCGATGCGAGACCTCGCGGTGGTCTC | 35-54 |
| ID NO: 34) | GAC (SEQ ID NO: 64) | |
| | GTCGAGACCACCGCGAGGTCTCGCATCGATCGT | |
| | GCC (SEQ ID NO: 907) | |
| SRR8543989_2137428_34\|M (SEQ ID | GGCACGATCGATGCGAGACCTCGCGGTGGTCTC | 35-54 |
| NO: 35) | GAC (SEQ ID NO: 64) | |
| | GTCGAGACCACCGCGAGGTCTCGCATCGATCGT | |
| | GCC (SEQ ID NO: 907) | |
| SRR8543995_2079833_6\|M (SEQ ID | GGCACGATCGATGCGAGACCTCGCGGTGGTCTC | 35-54 |
| NO: 36) | GAC (SEQ ID NO: 64) | |
| | GTCGAGACCACCGCGAGGTCTCGCATCGATCGT | |
| | GCC (SEQ ID NO: 907) | |
| SRR8544146_618080_5\|P (SEQ ID | GGCACGATCGATGCGAGACCTCGCGGTGGTCTC | 34-48 |
| NO: 37) | GAC (SEQ ID NO: 64) | |
| | GTCGAGACCACCGCGAGGTCTCGCATCGATCGT | |
| | GCC (SEQ ID NO: 907) | |
| SRR6963591_1323413_2\|M (SEQ ID | TAAAACACCTGCGAGATGGTTTATGAATCTCGA | 38-43 |
| NO: 38) | C (SEQ ID NO: 66) | |
| | GTCGAGATTCATAAACCATCTCGCAGGTGTTTT | |
| | A (SEQ ID NO: 909) | |
| SRR7976138_3273187_71\|P (SEQ ID | GGATCAACCTCTGCGGGAACGAGCGTGGTTCCC | 24-45 |
| NO: 39) | GAC (SEQ ID NO: 67) | |
| | GTCGGGAACCACGCTCGTTCCCGCAGAGGTTGA | |
| | TCC (SEQ ID NO: 910) | |
| SRR7976138_3273187_70\|M (SEQ ID | GGATCAACCTCTGCGGGAACGAGCGTGGTTCCC | 24-45 |
| NO: 40) | GAC (SEQ ID NO: 67) | |
| | GTCGGGAACCACGCTCGTTCCCGCAGAGGTTGA | |
| | TCC (SEQ ID NO: 910) | |
| 3300012943\|Ga0164241_10000441_4 | GGATCAACCTCTGCGGGAACGAGCGTGGTTCCC | 24-45 |
| 0\|P (SEQ ID NO: 41) | GAC (SEQ ID NO: 67) | |
| | GTCGGGAACCACGCTCGTTCCCGCAGAGGTTGA | |
| | TCC (SEQ ID NO: 910) | |
| 3300012943 \|Ga0164241_10000441_3 | GGATCAACCTCTGCGGGAACGAGCGTGGTTCCC | 24-45 |
| 9\|M (SEQ ID NO: 42) | GAC (SEQ ID NO: 67) | |
| | GTCGGGAACCACGCTCGTTCCCGCAGAGGTTGA | |
| | TCC (SEQ ID NO: 910) | |
| 3300031949\|Ga0214473_10000008_3 | GTAGCAATCGATGCGAGACCTCGCACTGGTCTC | 35-44 |
| 52\|M (SEQ ID NO: 43) | GAC (SEQ ID NO: 68) | |
| | GTCGAGACCAGTGCGAGGTCTCGCATCGATTGC | |
| | TAC (SEQ ID NO: 911) | |
| 3300031965\|Ga0326597_10001868_1 | ATGACAATGCCAGCCCAGCGGCCTGAAAGCTGG | 35-38 |
| 2\|M (SEQ ID NO: 44) | GAC (SEQ ID NO: 69) | |
| | GTCCCAGCTTTCAGGCCGCTGGGCTGGCATTGT | |
| | CAT (SEQ ID NO: 912) | |
| 3300006795\|Ga0075520_1037237_1\| | TACGGCACCACAGTGCATCGAATAGATGCAAC | 31-39 |
| M (SEQ ID NO: 45) | (SEQ ID NO: 70) | |
| | GTTGCATCTATTCGATGCACTGTGGTGCCGTA | |
| | (SEQ ID NO: 913) | |
| 3300014491 \|Ga0182014_10039677_1\| | GTACAACGAACTGAAAGTGCGCCGAACAGGTGC | 34-41 |
| P (SEQ ID NO: 46) | AAC (SEQ ID NO: 63) | |
| | GTTGCACCTGTTCGGCGCACTTTCAGTTCGTTG | |
| | TAC (SEQ ID NO: 906) | |
| 3300014491 \|Ga0182014_10071250_1\| | GTGTACGGCACCAGAGTGCATCGAATAGATG | 40-45 |
| M (SEQ ID NO: 47) | (SEQ ID NO: 71) | |
| | CATCTATTCGATGCACTCTGGTGCCGTACAC | |
| | (SEQ ID NO: 914) | |
| 3300017975\|Ga0187782_10004712_9\| | GTGTTCACGCTTGCGAGGACGGATTGCGTCCTC | 35-44 |
| P (SEQ ID NO: 48) | GAC (SEQ ID NO: 72) | |
| | GTCGAGGACGCAATCCGTCCTCGCAAGCGTGAA | |
| | CAC (SEQ ID NO: 915) | |
| 3300017975\|Ga0187782_10004712_9\| | GTGTTCACGCTTGCGAGGACGGATTGCGTCCTC | 35-44 |
| M (SEQ ID NO: 49) | GAC (SEQ ID NO: 72) | |
| | GTCGAGGACGCAATCCGTCCTCGCAAGCGTGAA | |
| | CAC (SEQ ID NO: 915) | |
| SRR7027835_762984_1\|M (SEQ ID | GTAACAGTGGCATCACAGTGCATTGAATAGATG | 34-46 |
| NO: 50) | CAAC (SEQ ID NO: 55) | |
| | GTTGCATCTATTCAATGCACTGTGATGCCACTG | |
| | TTAC (SEQ ID NO: 99) | |

TABLE 5
Non-coding Sequences of Representative CLUST. 133120 Systems

>3300017975|Ga0187782_10004712_9|M
TCTAATATTCTCTATGCCGAAGCCGGTTCGCGATGGATCAACCGTTGGGAGAGTGGCCAACTTGAAGCTGAGATGAAGCGTGCAAT
CGGGAAAATGTACCAGCGTATGCGTCTCGCGGAGAAAGTCATTGCACCCAAAAGAAGGCGTTAAGTAAATTGTGAGTGATCTACCG
CCCTGCCGCGTTCAAAGCATTCGCGCAAGCATGAACGCGCTGTACTGGACGCTGATCGTGGCAATCCTGGTTGGGATAGCGGCCGT
CAGCTTCACGCTTGGCGTCAAGCGTATGTACAATTGGGCGCACGCTCCGCATGCTCCGTCCGTTGTGGATTGCGGACGAGATTACG
ACGACGTGCGATGCCAGCTTTTGTACCTAGAGGACGTTTACTTGTGCGTGCTGAAAGACACTAGGCACGAGCCTTCGGAAGGCTCC
GGTCAAATACAAACGCTACTGATGCACGTCAGAAGATCGGCACAGAGGTAGGCGCGTTGGCGCCTTGCTCTTGACAAGTGCGCGAT
AACAACGTACTCTCGACAAGCGAATAAGTGGCAGCAGACGGGCGTTATAGACCCGAAAGGAAAACCACGTGGCTACCAAAGTCTTG
AAGTTCTATTGCGTACCCGCCTCTGCCCGAGATAACCAGCTCCTCTGCGAACAAGTGAAGCTCGGCTCGATGTACAGACGCGATCT
CGCTTGGATCGAGAATCGCGCACGCGAGCTGCGCCGTGCCATTGCCGATTGGCCTGCGCGTGATGTACCCATCGAAAAGCGTTCAG
AGTGGTGGAAGTCTGATGTCGGTCGCGCGGCCTCCAAAGCATGGCACGACCAGGATCGTACAGCTGCACGCAATCTCCTCGCCGGT
GCGAGCGCCATCGCGCGCGGTGAGAACGGGTGGCCGCTCGAAGCAAAGGTTTCGAAGGCTTCCAAGAAAAAGGTGGTGCTACGCCG
CACGCGTAAGCGCATTGTGGTGTGACCGCTCGCAAGAGCGGTGTGAAGTAACTGGTTTTGCTATTGATCTCTGACAACCTCGACAA
AGCGCCTGACTAGCCGTGACTTCCTATTTGCCTAGGGCTCTACACGAGCGGATTACGCTCGTAAACGAGGTTTTGATGCTTGCACG
GGCGGATTGCGCTCGTGACCTCACGCTCCTCGGTCACTTATTTCCGTTGCATCGCACTGCCCGCAATCAGCACGAGCCCTAGCCCG
ATCACACCCAACGCGACCACGACGATCAAGCCTTCAATCCCAACGTTCGCGTAGCGCGGCGATCGTCAGTTCCAACGGGTACTGAT
CGAATCCGTGCCAGCCGTAGTGGCCAGCGGCATCCCAAACGGCGTTTGCACGCTCACTCGCAGTCTTCAAGTTCTTTGCGTCATAC
CGTTCTAGGTC (SEQ ID NO: 73)
>3300006795|Ga0075520_1037237_1|M
TCTCACACCCCGGAGTCCCAACCGACCCCGAGATTCCCGAATGGCTCAAGGCGTCGATTGCGCGACAGAGATCCCTCTGGAATCGG
TTGGCATACTTCTGTCGCGAAGCAAGACGGAAGTGCTCGCCAGCGCCCACGGCTGAAATAGTGGCGTTTGTTCGAGGGACCATTCT
TCCGGAGATCGATGCGTTCAATGACGCTCTCGGGCGGTCGAAGGCAAAGATCAAGCATCCTGCCAAGCTCAAGACGGAGATGCCGG
GGATCGATGGACTGTGGCACTTCGTTGGGCAGCTCCGGTGCAACCACCTTAACCCTGGCACCGGGAAAGAGTACTTCACCTGCGAG
AAATGCGAGCGGCAGATCAAACAAGACCACAATGCCGCGGTCAATATCTCGCGCTTCGGCTGTGATCCAGAACTGGCAGAGATGGC
CGCCTCTGCTGGCTGAGTCTTTACCTATAGCTCTGCTCGAATCTCAAGCGAGCATTTATTGCTCGGGAGGTTCGCGCAGGTTGTTC
CCGCACATAATAAAGGAGATTGTGAAATATCAAGGGCGATGTCGTCAAATCAAATACTCTGCGGGGAGGGTTCGCGCAAACCATTC
CGTAGATCATTGCGGTCGAATAAGTTACAGTGAAGGAATCCATGCGATTGAAAACTATTGTAGTCGCGCTGGTGCTGCGTAC
(SEQ ID NO: 74)
>3300017971|Ga0180438_10084474_2|M
CCACAACAAGGTGTTCAAGCAGCTCGCCGAAGAACTCGGGCTCGCCGTCGAGAAACAGGGTGCCCGGGGGTTGGCGGCCACCTCCG
TGCCCGCGGAAACCGCGAAGCGCTGGCAGAAGACCATCAAAGAACTCGACAAGGCCATCAAGACGTTTCGGTTCGCCGAGGGCCCC
AGGGGCGGCAGCAAAGGTGAGAACCGCCAGCTGCTGGCCATCTGCCAGTGCCGCCGCAAGATCCGGTTGTCCCGGGAGGCCTACGA
CGCTGGCCCCATCATCTGCCGGGTATGTGAACGACACTTCGAGATAAAGGAGTAGAAAAATGAAAGTATTCAAGTACGGTGCTCGA
CCCGTAAACCAGGAGAGCCTCATTCGTGATCAAATGAAGCTCGCCCGAAACTACTACAACAAGCTCGTTGAAACGGAGAACAAGAG
ACGGCAACAGGCCTGGGGCAGCGAAAAGCCTCCTCCTGCGACCATGAAGAGGTAACACCTACGAGACGGTTTATGAGTTTCGACTA
TACTACTGATAATCTGCCCCAACTTGTCTCATGGATAAAACACCAGCGAGATGGTTTATAAATCTCGACAGACTCGCGGATGGAAG
AGAACTCGCGACACTCGCGGGGTAGAACACCAGCGAGATGGTTTATGAATCTCGACAGGTCGAAGTAGATACCAAACCTCTACCAT
AACCTTACGTAATTCTTGACTTTTATTTGCACTTGCGTAAGTGAATACCCGAACAGTACAATCGTCTACATACCAGCGGCTTAGAG
GAGCCAGAAAGGAGAACTAGATGTCAGCAGCCTTATTAGCCCCGCTTTGGAGGAAGCCGTACATCCTCGGGGGCAACGCCAAGGTC
ACCTTGGTGTCTACCAAGTCCGAGTGCCGGTTCACATTCCGCATCCGCGCCAAGGAAGTCGAGAAAGATCGCGTTCTCCACTTCGT
CAGCGTGCTGACGGGGCCTGAGAACACGACCGACTACTCCTTCCTGGGAACCATCTTCGACGGCGACAAGTTCGTTCACGGTAAGA
AGTCGAAGATCACGGCCGACGCGCCCAGCGCGAAGGCCTTCTCCTGGCT (SEQ ID NO: 75)
>SRR8543844_2229942_334|P
CAAGTCACTCATCGTGATCTTTCTGCGCTTCATCTCGGCGCGGAGCCACGACTCTTCGGTGTCGGTGACGCGCGCACCGATGTAGT
GGCTGAGTTTCTCCGCGGCTTGTTGCTTCGGCTCGAACATTGCGTCTTGCAAAACGTCTATCATCCTGTTAGCTGTTCGTCAATGC
AACAACAGCACGTGCGACAGCGGGGCGCCCAACAATGACGATGCGCGTGTACAAGTTCCTCTGTCTCCCGGCGACGGCCGCCGACG
ACCATTGCTTGCGCGAGCAGGTGCGCCTCGGCGCGCGCTACCGCCGACAGATCGCGCTCATCGAGAACCGCGAGCGCTTCCTCCGG
CGCGCGCTCATCCTCGCCGCCGCGAGCGCCGTCGCGCGCGGCGAGAGCGGGTGGCCGCTCGACCCCAAGGTTCCGGCACCTTCGAC
GAAGAAGAAGGCCCCGGCGCGGCGGAACCGGCGGAAGGACCAGCAGCCGCTCGCGCCGAGCCCCGCAACGCCGTAGGATCGTTCGT
TCCATGCTTATCCGGTTAACACCCCAAACGTGATCAGGGAGACATCAAATGGCATCGCGACTCGAACGTGAACTTAAAATCATCCG
CTCCGAAATGGGCGAGCTTCTTTCCGCCTCTCTGGGCAAGCGACCGTCCGTGATCAACGTCGGCGGAACGATTCAAGAACTCACCC
CAACGGAAGCTCTCGCTGAATGGCGAGCCTCTGCGAAGCGCGCCGAGCGAAAGATCAAGGAGATCCGTCGCGCCCGCGAGGGGTGA
CAACCGGATAAGCATGGTTCGTTCTCTGACAATCGAAACCTACGGATTGTCCTCGGCGGAGGGATTCGGGTTCGTCGGCACGATCG
CGGGCGCGCTATGGTGAGGTCATGGC (SEQ ID NO: 76)
>3300020171|Ga0180732_1000630_46|P
CGCATGCGCCTGATGCAGATCGAGTGGGAGAAGCAGCCGAAGTTCACGCTCGGCGCCTGCCGGCCGCCCGGGGTGCCGCACACCTG
GCCGCCCAAGTCGGCCGGGCCGGCCTGGATGGTCGTGTTTCGGGTGAAGGGGACGAGCAAATGATGAAAGTATTCAGTTTTGGTGC
TAGGGCTCGGTCACTTGACGCCGAGGTCCGCGAGCAAATGCGCCTCGGTCGAGAGTGCTATAACGCGCTGGTCGAAGTGGAAAACG
CTCGCCGTCACTTGGCCTGGGGTGGGGACATCCCCCCAAAGCCACCGCATCCGCTACCACTGACCCAGGATGGCGACGAGAACGAC
GATGAGACTGCGTGGCTAGATCGCGACCTCGTAAGCACTCGTAACATGCTGGTTGCCTACGCTGCGGAGGAGAGGCGGCGACCAAC
CGCTCGCAAAACCGCTGCTAGATTCGGGAAACGACACAAGACCTCGACGAGCCTCGACAATGCCAGCCCAGCGGCCTAGAAGTTGG
GACAAGTTGGCTCCGACTGACAATGCCCACGAAGTCGGGCCTCGACAATGCCAGCCCAGCGGCCTAGAAGTTGGGACGCCGCCCCT
GTCCTGCGGCTCCTCGTCCGTCGCCGTTCGACAATGCCAGCCCAACGGCCTAGAAGTTGGGACAACCCGTCCATGTAGTCGCGGGC
CGCTTCGCTGACTGATGATGCCAGCCCAACGGCCTAGAAGTTGGGACATGTTTAGACGCGCCACCCAGCCCGCTCCCGATGCGGCT
GATGATGCCAGCCCAACGGCCTAGAAGTTGGGACGACGATGTACCAAGAGTGCCTGATGATGCCAGCCCAACGGCCTAGAAGTTGG
GACCGGGTGCCTCGGCCCACAGCTTCGCCGGCAACGCGGTCGACAATGCCAGCCCAACGGCCTAGAAGTTGGGACCATGGCCAGGA
TGCTGATGATGCCAGCCCAACGGCCTAGAAGTTGGGACAGAAGGCCACCGTCGTCGGTGGGCAGGTCGACCCGGCACCTGATGATG
CCAGCCCAGCGGCCTAGAAGCTGGGACTGCGATCCGTCGTGGTCCGCCATCGAGATCGATGCCGATCTGATGATGCCAGTCCAGCG
GCCTAGAAGCTGGGACCTGTCAGCACTGTGGCCTGACCCATCCGTGCCTAGCTATCGACAATGCCAGCCCAGCGGCCTAGAAGCTG
GGACTACCACGGTGGGCACCGTGCTATTCATTGGCTACCCAATGAATATGGTGTATAGCATTATAGTCTTAGAAAAAAATCTTAGT
AAAACGCGGCGCAAAGGACGGTGACTAGTGGACGACCCCAGACTGGAAGTAGCGGCCCAGGCAATCGACAGGCCGGAGGCCCAGGT
CAAGGAGCTGTCGGACGCGCTGCCCGACGCCAAATACTTGAGCAATCTCGCGGCGCGGCTGAAGGACGTTCCCAAGACGTATGGCG
TGTTC (SEQ ID NO: 77)
>3300027740|Ga0214474_1000103_17|M
AATGGAATAGCCCGGACGGTACTTGACACGTGTAGAAAATGCTACTATTTATGTAGTAATCATTATGCCAACCCCATTTTTCCAGC
CAGGCGATCCGCGTTGCCACCGCGCCGGCAGACCCAAGGGGACAGCGCTCAAGAAAATGCGCAAGATGGCTGCCGAGGTGCTAGGC
GAGGAGCTGGAAACGCCGACTGGGCGTATGACCCGCGCAAAA (SEQ ID NO: 78)
>SRR7976138_3273187_70|M
CCCGTGGTCCTGACGTTGTTCGGGGGATATCGGGATGACCATCCCGAGAGCGTGCTGGGGCTCCACGCGATGGGCGTGGCCCGCGC
GCTCTACTGGCTCGCGGGCGTCCGCAAGCTCGCCGACTGGCGCGCGGATGTTCGCCCGCGCTGAGTCTCTGCGGAGGCGAGTTTGG
CCTCCGACACCGAGTCCGCACGTTATCACCGGGTTGGCGGTCGCCGACCCAGAAGGGATCGAGATCATGATGTGCTTCCAATACTG
GGCCGAGCCCGTCGACGAGACGAGCAAGGCACTCCTCGCTCACCAACTCCGCGAGGGCGCGATGTACCGCCGCATCCTCGCCACGC
TCGAAAATCGCGATCAAGACCACAGCGCCGCGAGGACTCTGCTCGCGAGCGGTCTGGTGATGGCGCTCGAAGGCGCAGCGACGAAG
GGTCGAACCCCGCGAAAGATGGGGGCGCGGCGTACGCGCAAGAAGGCCCAGGTCGAGATCGCTCGCGACGCGTGATACAAGTTCGC
TCTCTGACAACCGAATAGGTCATCGCA (SEQ ID NO: 79)
>3300014491|Ga0182014_10071250_1|M
TCCCTCTGGAATCGGTTGGCATACTTCTGTCGCGAAGCAAGACGGAAGTGCTCACCAGCGCCCACGGCTGAAATAGTGGCGTTTGT
TCGGGAGATCATTCTTCCGGAGATCGATGCGTTCAATGACGCTCTCGGGCGGTCGAAGGCAAAGATCAAGCATCCTGCCAGGCTTA
AGACGGAGATGCCGGGGCCCGATGGACTGTGGAACTTCGTTGGGCAGCTCCGGTGCAACCACCTTAACCCTGGTACTGAGAAAGAG
CACTTCACCTGCGAGAAATGCGAGCGGCAGATCAAACAAGACCACAATGCCGCGGTCAATATCTCGCGCTTCGGCTGTGATCCAGA
ACTGGCAGAGATGGCCGCCTCTGCTGGCTGAGTCTTTACCTATGCCCTGTGCGGATCTCAAGCGAGCATTTATTGCTTGGGAGGTT
CGCGCAGGCTATTTCCGCTCATAACAAAGGAGATTGTGAAATATCAAGGGCGATGTCGTCAAATCAAATACTCTGCGGGGAGGGTT
CCCGCTAACCATTCCGTAGATCATTGCGGTCCAATAAGTTACAGTGAAGGAGTTGGATTTTCACGCTGCCGCTTGGAAATTGAGTT
TGTAGCGGTATCCGCGCCCAATATTTTGCGCAGAATCAGACTCAGGCTAAAAGTCTTCTCCCGTGAGAGGCACG (SEQ ID NO:
80)
>SRR2657585_2135055_9|M
TCTCATGTACGCCACCGTCGATCCTCGGGCGAACGGAGGGTTTATGGTGACCTACGGTTGGCGAAACAAAGATGGAACCGCGGAGG
TGATGCGAGAGCACTTCTCTGACCTCGAAGATGCCAAGGTTGATGCGGCAGACTTTATGCTCTGCCGCGGGGACTGGGAAGAATGA
GCAGGGCCAAATGCCCAAAAAAGCGCTTGTTGGCTCACGCCCGAGGCGAAGACGAGTGCTGGCTATGGCCAGGAGCGGACTCTGGC
AACGGCTACGGGAGGATCTCAGTGGACGGCAAAACAAGAGCGACTCACATCGTGGCCTACGAGCTGTTTACCGGTACTCGTGTGCC
GCCTGGAATGGTGCTCGACCATCAGTGTCGAAATCGACTGTGCTACAACCCGAGATGCCTCAAGCCCATTCCAGGCATTGTCAACA
CGCTCATCGGTGAGGGACCTACTGCAGCCAATGCGCTCAAGACGCATTGCTGTCGCGGCCACGAGTTGGCCGGTGAGAATCTCATC
ACTAGGACTCGCGAAGGTCGGCGGCCAAGTCGTGAGTGTCGTGCCTGCCGCAGGATGCGCGGCAGAGCGCAGGAGATGTGCGTATG
AGCAAGTGCGAGGGCTGCGGGGAAGATCATCTCGTCCTAGGCGCTTGCATACTGAAGGCAGCGATCTACGAGGCTCCCTGTATCTA
CTGTCATGCGCGGCCAGGAGAACTGTGCGTAACCTGCACCGGCAAGGAGAGCCCGCAGTGGCACATGCAGCGCGTCGGGGCTGGCC
ACACGCTACTAGTCGCGAGAGGAACACCGTGCCTTGCTTCTCGCCATATACAGAGAATGCGCGGAAACCGTCGCAATAAAGGGCAC
ACCAGACAGAACTGGCATCGCAACAAGGAACGACTGAGTCATGGAAAGAGAGTGAAATAATATGGCTACACGCGTCTACAAATTCG
GAGCCCGCTCACCAGGTGAGGCCACTCTTCTGCGCGCCCAGATGAAACTGGGCCGCGAGTACTACAACGCCCTCGTCGAGGTCGAG
AATAAGCGCCGGCAAAAAGCCTGGGGCGCCGAGACGGTGCCAGCACCACCGCACGAGGATTGCAAGTTGCCCAAGTGCCCCGAGTG
TCGCGACCATTGGCGAGCG (SEQ ID NO: 81)

### Example 2—Functional Validation of Two Engineered CLUST.133120 CRISPR-Cas Systems

Having identified components of CLUST.133120 CRISPR-Cas systems, two loci were selected for functional validation: 1) a locus from the metagenomic source designated 3300027740 (SEQ ID NO: 1) and 2) a locus from the metagenomic source designated 3300017971 (SEQ ID NO: 2).

### DNA Synthesis and Effector Library Cloning

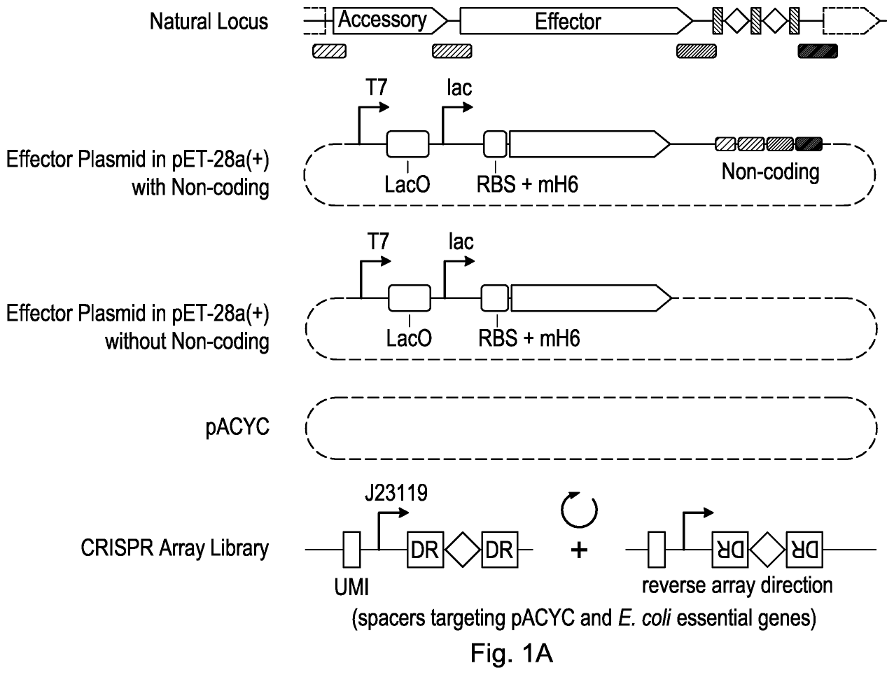

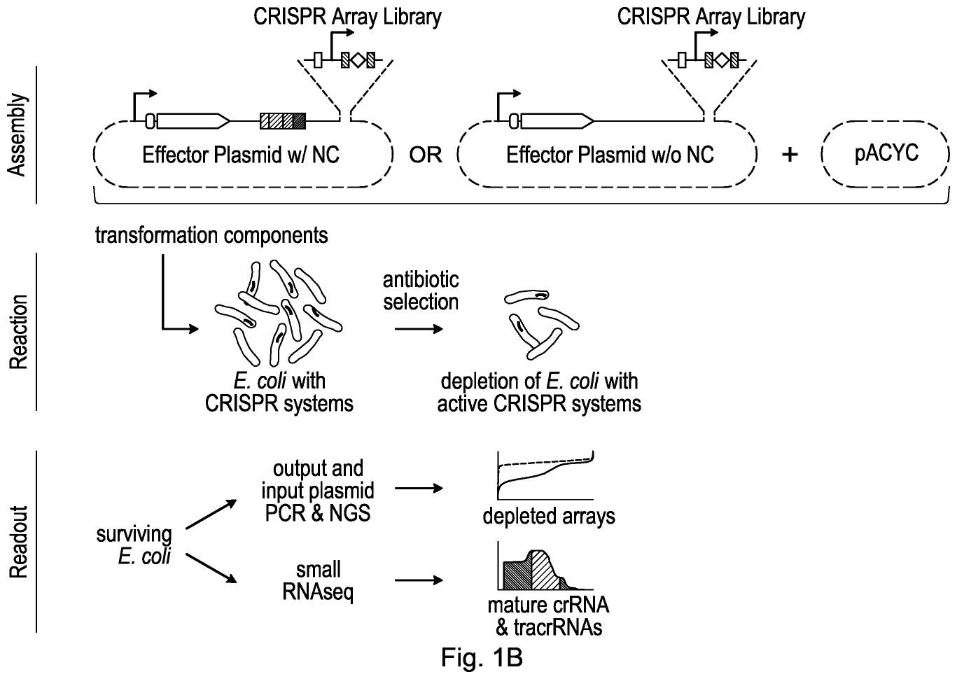

To test the activity of the exemplary CLUST.133120 CRISPR-Cas systems, systems were designed and synthesized using a pET28a(+) vector. Briefly, an E. coli codon-optimized nucleic acid sequence encoding the CLUST.133120 3300027740 effector (SEQ ID NO: 1 shown in TABLE 3) and an E. coli codon-optimized nucleic acid sequence encoding CLUST.133120 3300017971 effector (SEQ ID NO: 2 shown in TABLE 3) were synthesized (Genscript) and individually cloned into a custom expression system derived from pET-28a(+) (EMD-Millipore). The vectors included the nucleic acid encoding CLUST.133120 effectors under the control of a lac promoter and an E. coli ribosome binding sequence. The vector also included an acceptor site for a CRISPR array library driven by a J23119 promoter following the open reading frame for the CLUST.133120 3300027740 effector. The non-coding sequence used for the CLUST.133120 3300027740 effector (SEQ ID NO: 1) is set forth in SEQ ID NO: 78, and the non-coding sequence used for the CLUST.133120 3300017971 effector (SEQ ID NO: 2) is set forth in SEQ ID NO: 75, shown in TABLE 5. Additional conditions were tested, wherein the CLUST.133120 3300027740 effector (SEQ ID NO: 1) and the CLUST.133120 3300017971 effector (SEQ ID NO: 2) were individually cloned into pET28a(+) without the non-coding sequence. See FIG. 1A.

An oligonucleotide library synthesis (OLS) pool containing “repeat-spacer-repeat” sequences was computationally designed, where “repeat” represents the consensus direct repeat sequence found in the CRISPR array associated with the effector, and “spacer” represents sequences tiling the pACYC184 plasmid or E. coli essential genes. In particular, the repeat sequence used for the CLUST.133120 3300027740 effector (SEQ ID NO: 1) is set forth in SEQ ID NO: 51, and the repeat sequence used for the CLUST.133120 3300017971 effector (SEQ ID NO: 2) is set forth in SEQ ID NO: 52, as shown in TABLE 4. The spacer length was determined by the mode of the spacer lengths found in the endogenous CRISPR array. The repeat-spacer-repeat sequence was appended with restriction sites enabling the bi-directional cloning of the fragment into the aforementioned CRISPR array library acceptor site, as well as unique PCR priming sites to enable specific amplification of a specific repeat-spacer-repeat library from a larger pool.

Next, the repeat-spacer-repeat library was cloned into the plasmid using the Golden Gate assembly method. Briefly, each repeat-spacer-repeat was first amplified from the OLS pool (Agilent Genomics) using unique PCR primers and pre-linearized the plasmid backbone using BsaI to reduce potential background. Both DNA fragments were purified with Ampure XP (Beckman Coulter) prior to addition to Golden Gate Assembly Master Mix (New England Biolabs) and incubated per the manufacturer's instructions. The Golden Gate reaction was further purified and concentrated to enable maximum transformation efficiency in the subsequent steps of the bacterial screen.

The plasmid library containing the distinct repeat-spacer-repeat elements and CRISPR effectors was electroporated into E. Cloni electrocompetent E. coli (Lucigen) using a Gene Pulser Xcell® (Bio-rad) following the protocol recommended by Lucigen. The library was either co-transformed with purified pACYC184 plasmid or directly transformed into pACYC184-containing E. Cloni electrocompetent E. coli (Lucigen), plated onto agar containing chloramphenicol (Fisher), tetracycline (Alfa Aesar), and kanamycin (Alfa Aesar) in BioAssay® dishes (Thermo Fisher), and incubated for 10-12 hours at 37° C. After estimation of approximate colony count to ensure sufficient library representation on the bacterial plate, the bacteria were harvested, and plasmid DNA WAS extracted using a QIAprep Spin Miniprep® Kit (Qiagen) to create an “output library.” By performing a PCR using custom primers containing barcodes and sites compatible with Illumina sequencing chemistry, a barcoded next generation sequencing library was generated from both the pre-transformation “input library” and the post-harvest “output library,” which were then pooled and loaded onto a Nextseq 550 (Illumina) to evaluate the effectors. At least two independent biological replicates were performed for each screen to ensure consistency. See FIG. 1B.

### Bacterial Screen Sequencing Analysis

Next generation sequencing data for screen input and output libraries were demultiplexed using Illumina bcl2fastq. Reads in resulting fastq files for each sample contained the CRISPR array elements for the screening plasmid library. The direct repeat sequence of the CRISPR array was used to determine the array orientation, and the spacer sequence was mapped to the source (pACYC184 or E. Cloni) or negative control sequence (GFP) to determine the corresponding target. For each sample, the total number of reads for each unique array element (rₐ) in a given plasmid library was counted and normalized as follows: (rₐ+1)/total reads for all library array elements. The depletion score was calculated by dividing normalized output reads for a given array element by normalized input reads.

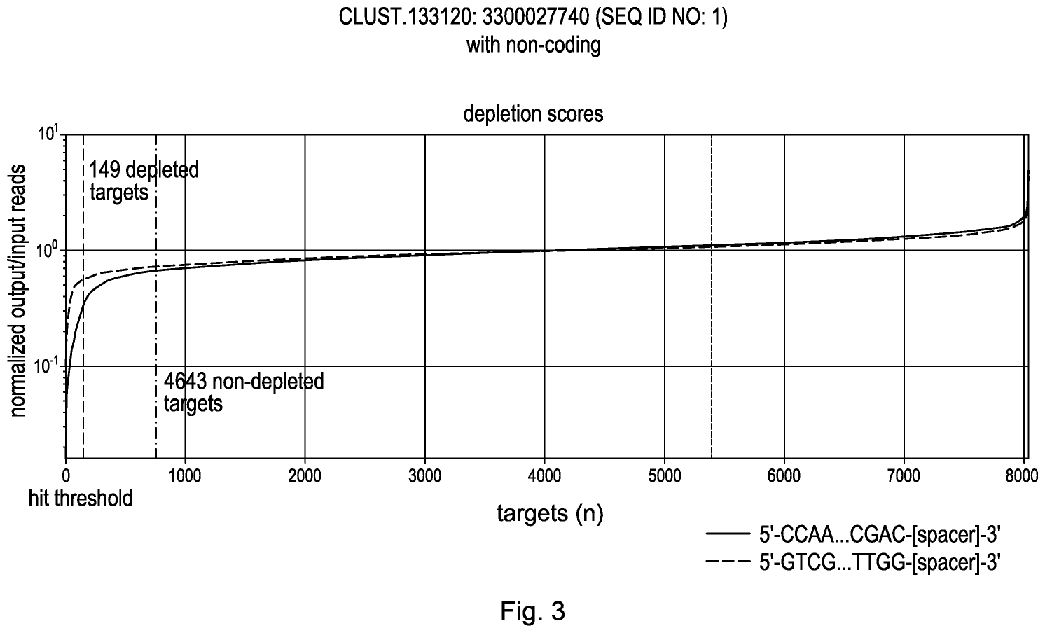

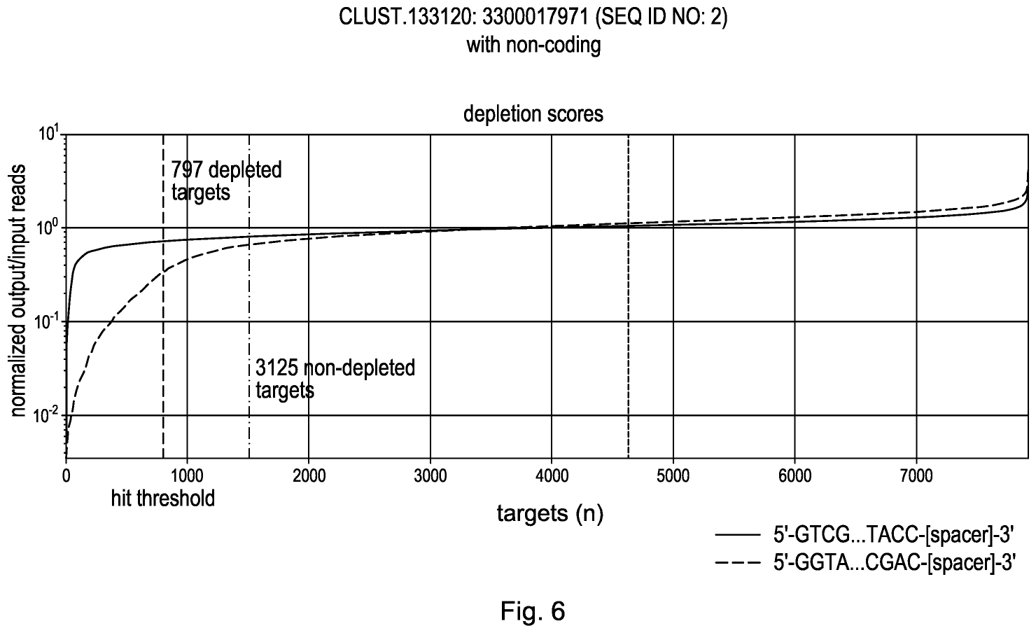

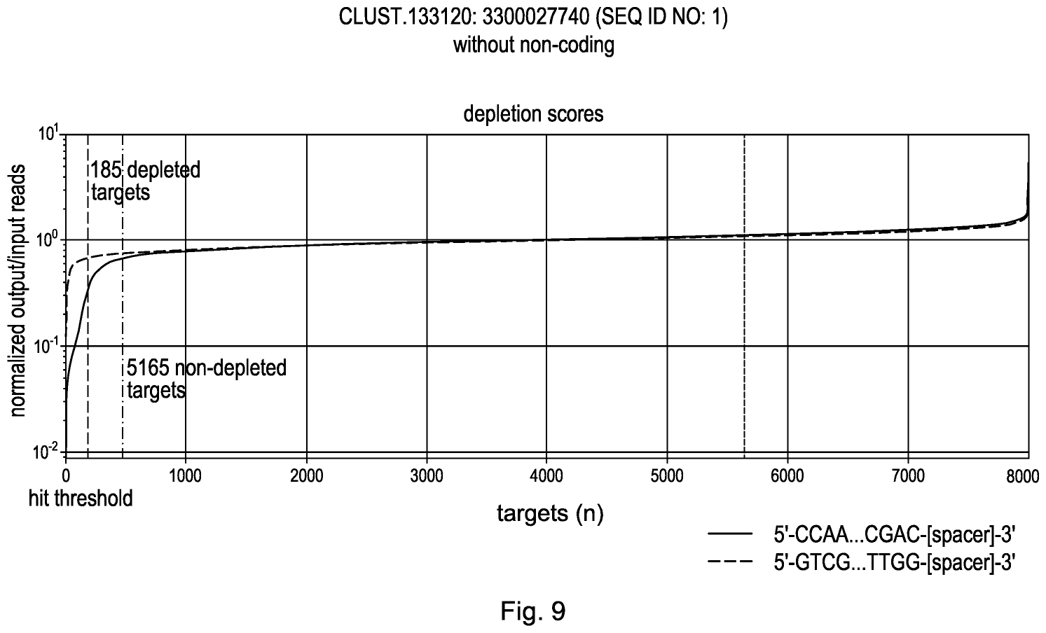

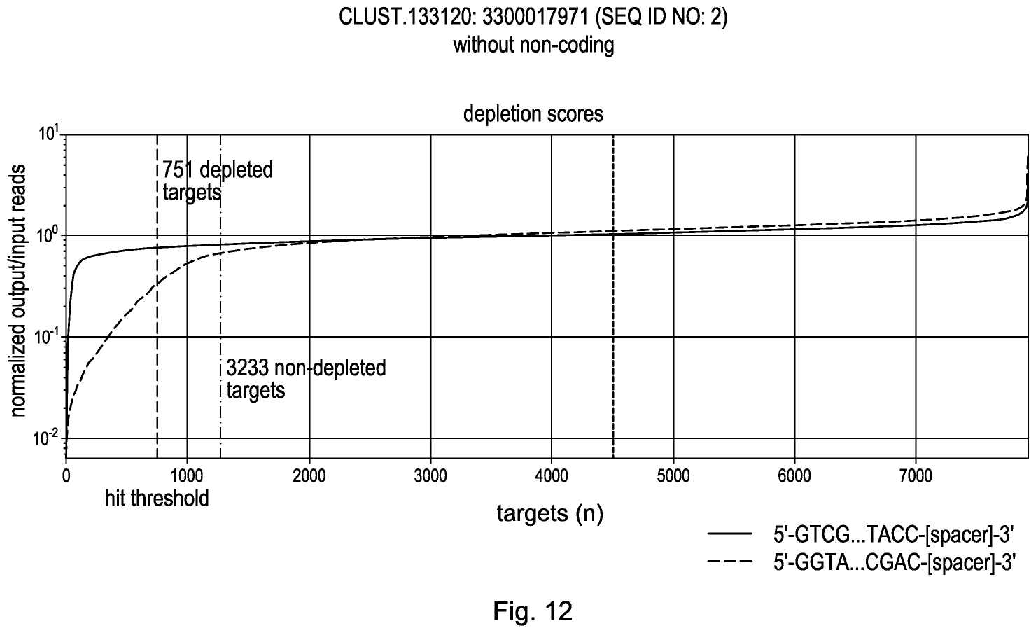

To identify specific parameters resulting in enzymatic activity and bacterial cell death, next generation sequencing (NGS) was used to quantify and compare the representation of individual CRISPR arrays (i.e., repeat-spacer-repeat) in the PCR product of the input and output plasmid libraries. The array depletion ratio was defined as the normalized output read count divided by the normalized input read count. An array was considered to be “strongly depleted” if the depletion ratio was less than 0.3 (more than 3-fold depletion), depicted by the dashed line in FIG. 3, FIG. 6, FIG. 9, and FIG. 12. When calculating the array depletion ratio across biological replicates, the maximum depletion ratio value for a given CRISPR array was taken across all experiments (i.e. a strongly depleted array must be strongly depleted in all biological replicates). A matrix including array depletion ratios and the following features were generated for each spacer target: target strand, transcript targeting, ORI targeting, target sequence motifs, flanking sequence motifs, and target secondary structure. The degree to which different features in this matrix explained target depletion for CLUST.133120 systems was investigated.

FIG. 3 and FIG. 6 show the degree of interference activity of the engineered compositions by plotting for a given target the normalized ratio of sequencing reads in the screen output versus the screen input. The results are plotted for each DR transcriptional orientation. In the functional screen for each composition, an active effector complexed with an active RNA guide will interfere with the ability of the pACYC184 to confer E. coli resistance to chloramphenicol and tetracycline, resulting in cell death and depletion of the spacer element within the pool. Comparison of the results of deep sequencing the initial DNA library (screen input) versus the surviving transformed E. coli (screen output) suggests specific target sequences and DR transcriptional orientations that enable an active, programmable CRISPR system. The screen also indicates that the effector complex is only active with one orientation of the DR. As such, the screen indicated that the CLUST.133120 3300027740 effector was active in the forward (5′-CCAA . . . CGAC-[spacer]-3′) orientation of the DR (FIG. 3) and that the CLUST.133120 3300017971 effector was active in the reverse (5′-GGTA . . . CGAC-[spacer]-3′) orientation of the DR (FIG. 6).

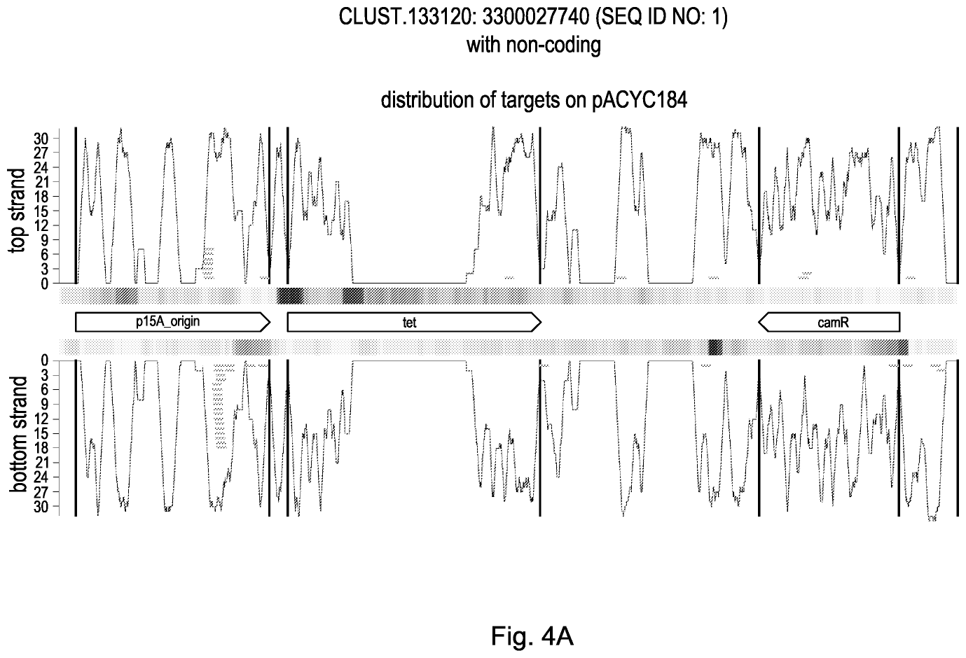

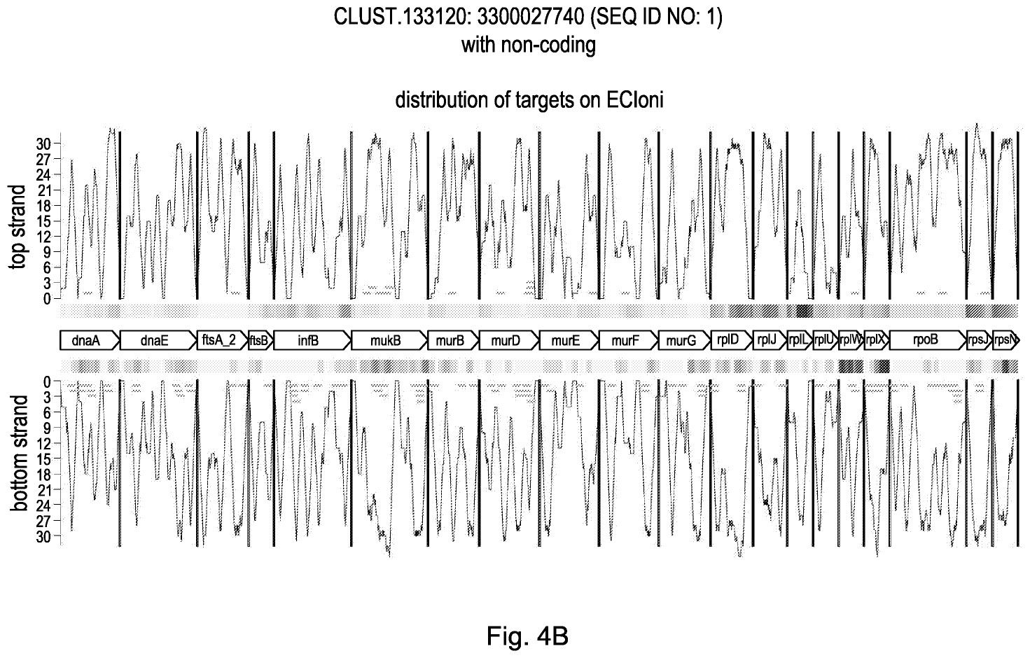

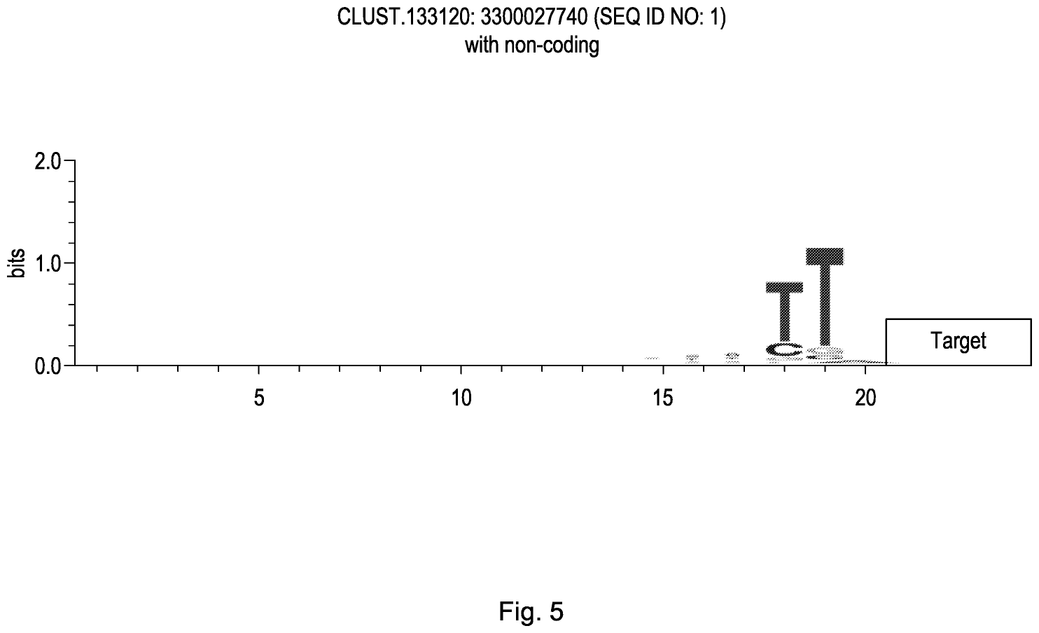

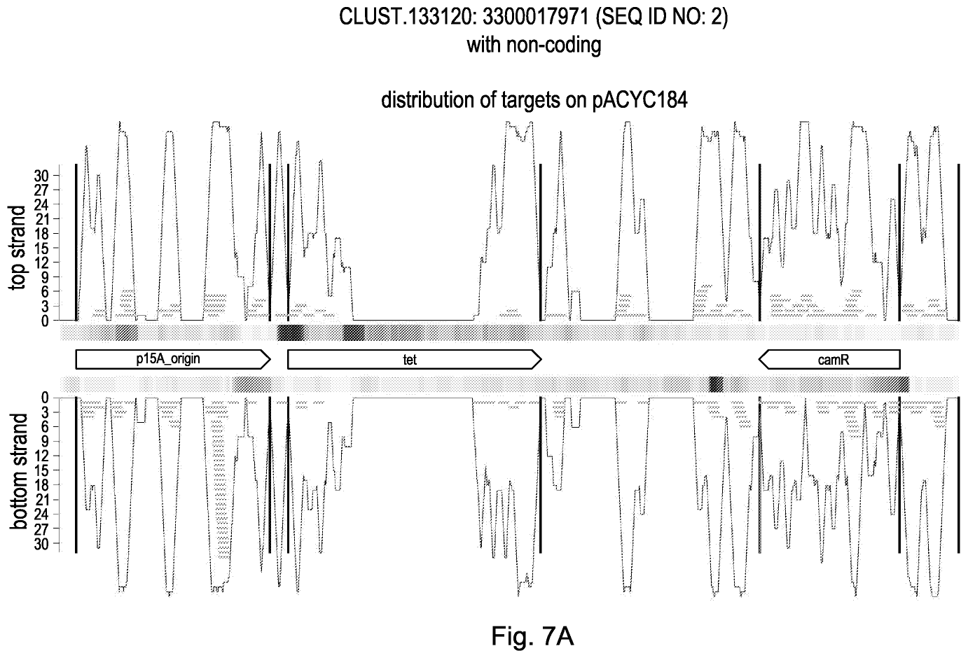

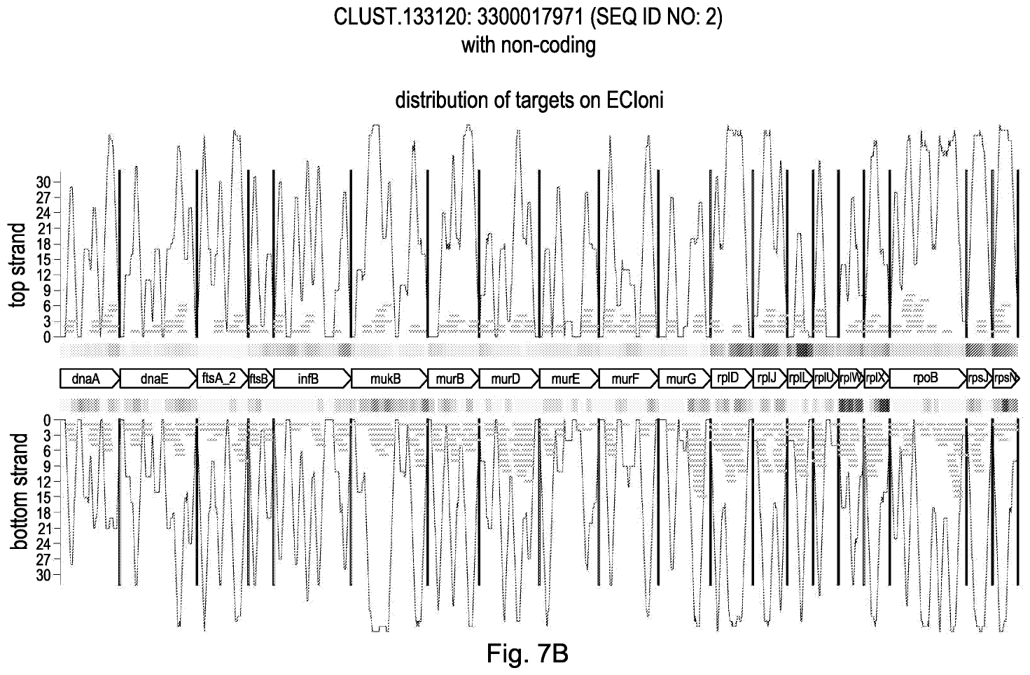

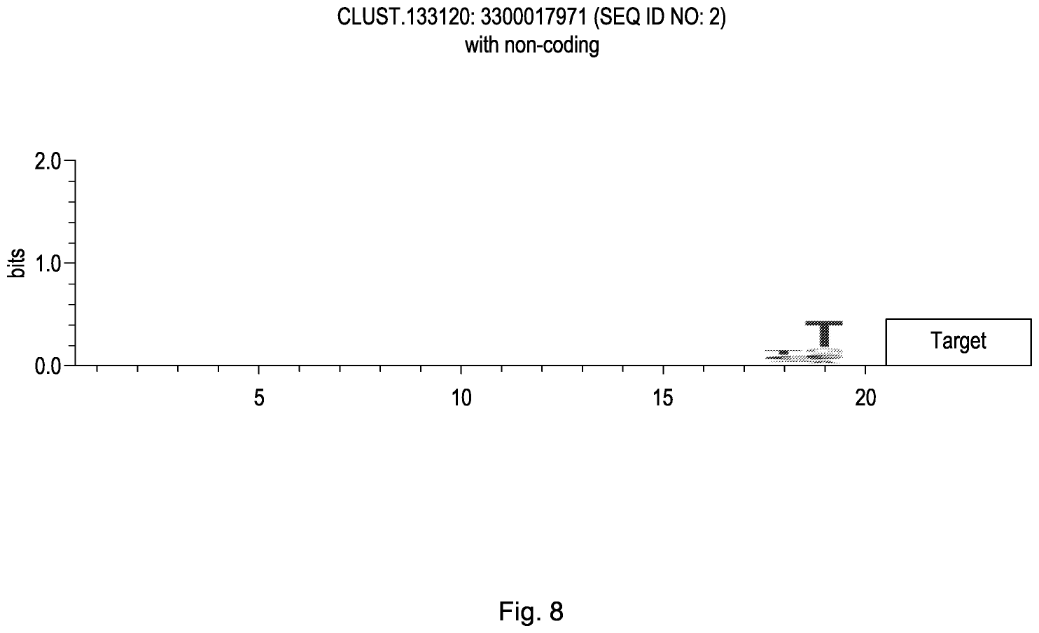

FIG. 4A and FIG. 4B depict the location of strongly depleted targets for the CLUST.133120 3300027740 effector (plus non-coding sequence) targeting pACYC184 and E. coli E. Cloni essential genes, respectively. Likewise, FIG. 7A and FIG. 7B show the location of strongly depleted targets for the CLUST.133120 3300017971 effector targeting pACYC184 and E. coli E. Cloni essential genes, respectively. Flanking sequences of depleted targets were analyzed to determine the PAM for CLUST.133120 3300027740 and CLUST.133120 3300017971. WebLogo representations (Crooks et al., Genome Research 14: 1188-90, 2004) of the PAM sequences for CLUST.133120 3300027740 and CLUST.133120 3300017971 are shown in FIG. 5 and FIG. 8, respectively. The “20” position corresponds to the nucleotide adjacent to the 5′ location of the target.

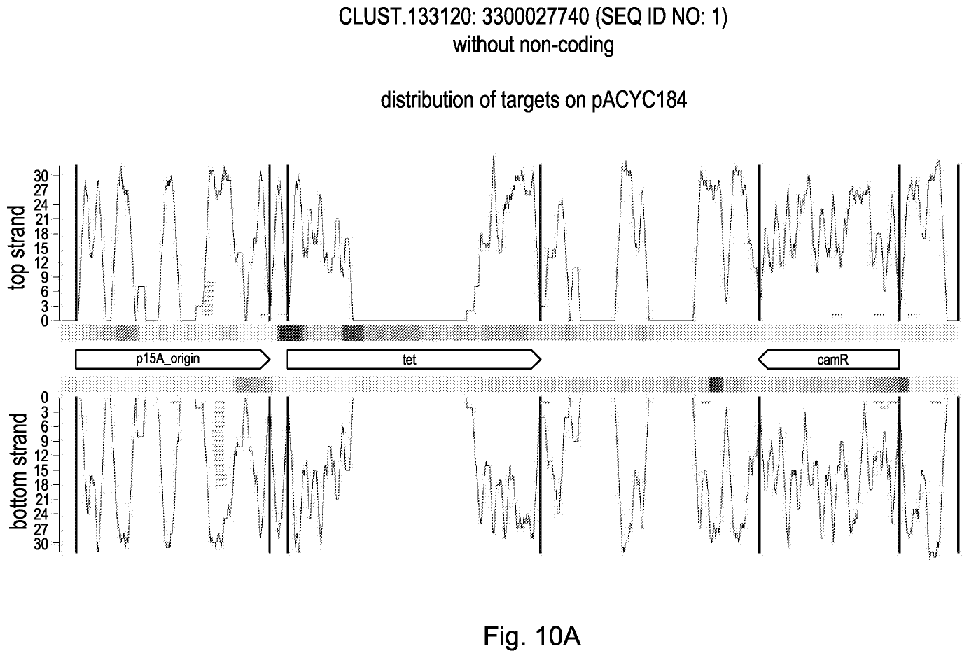

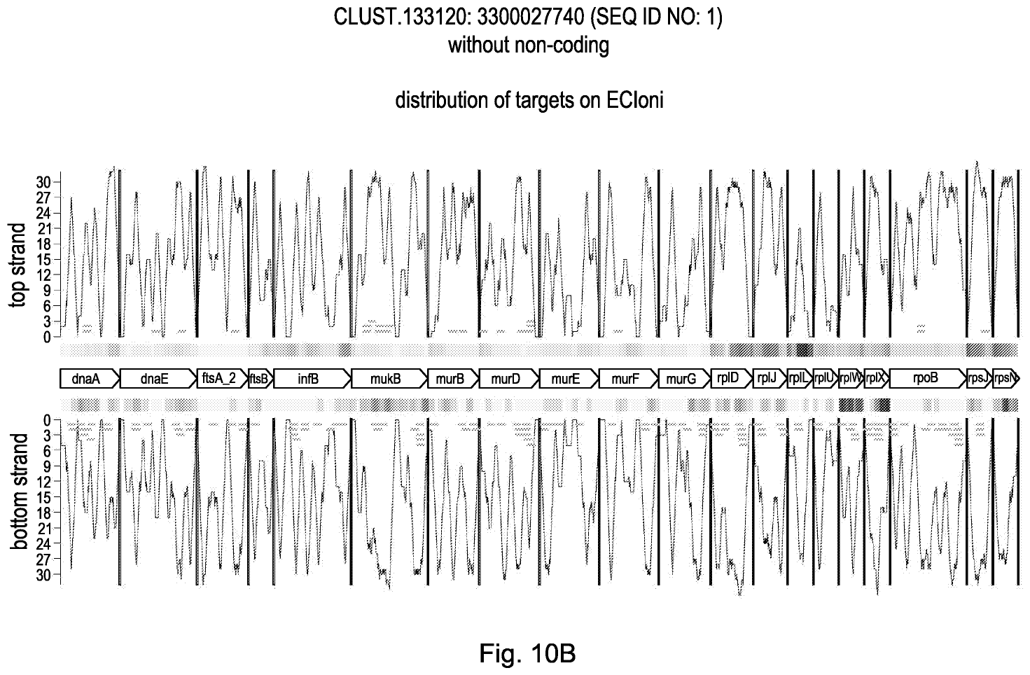

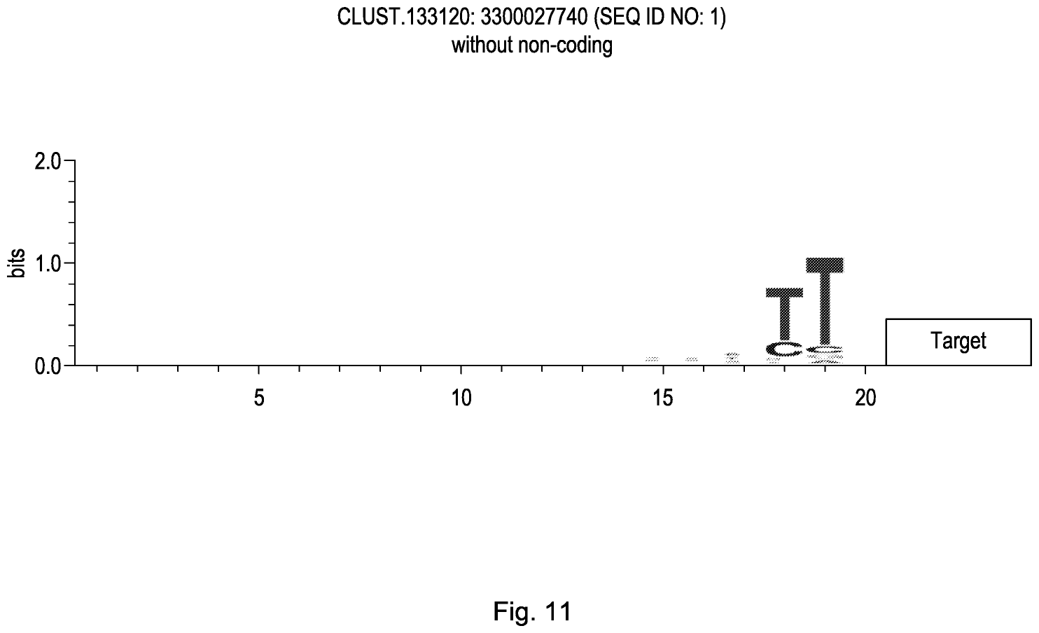

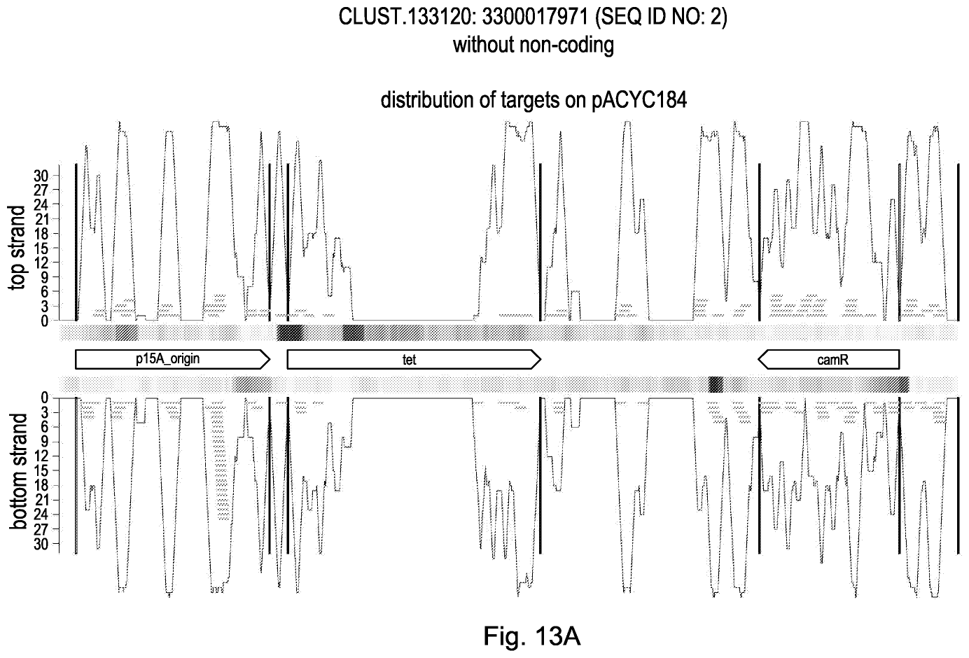

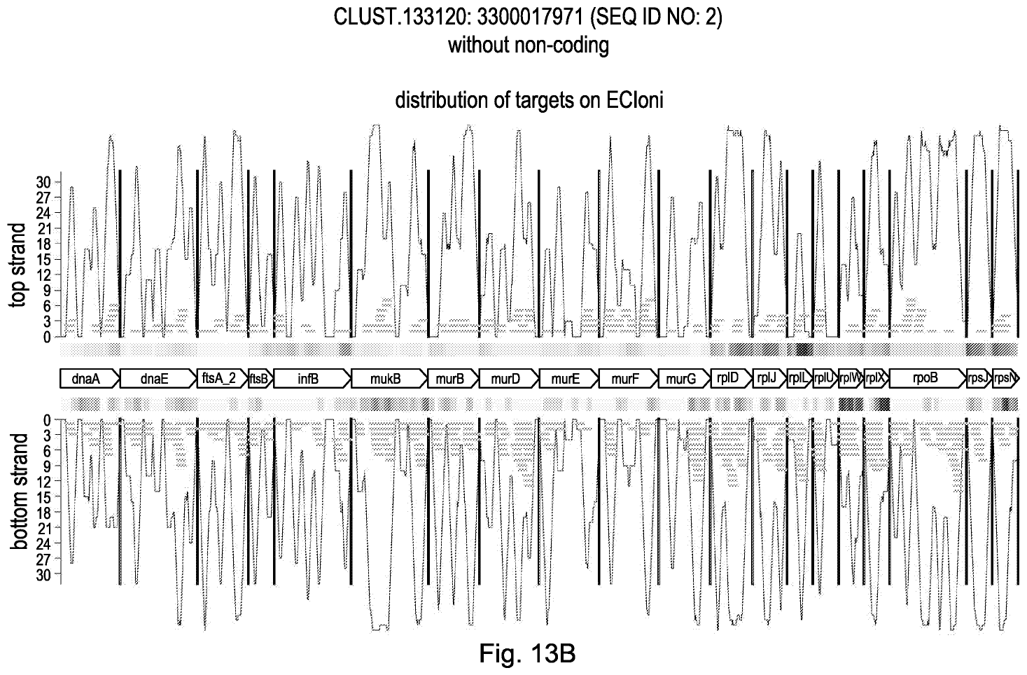

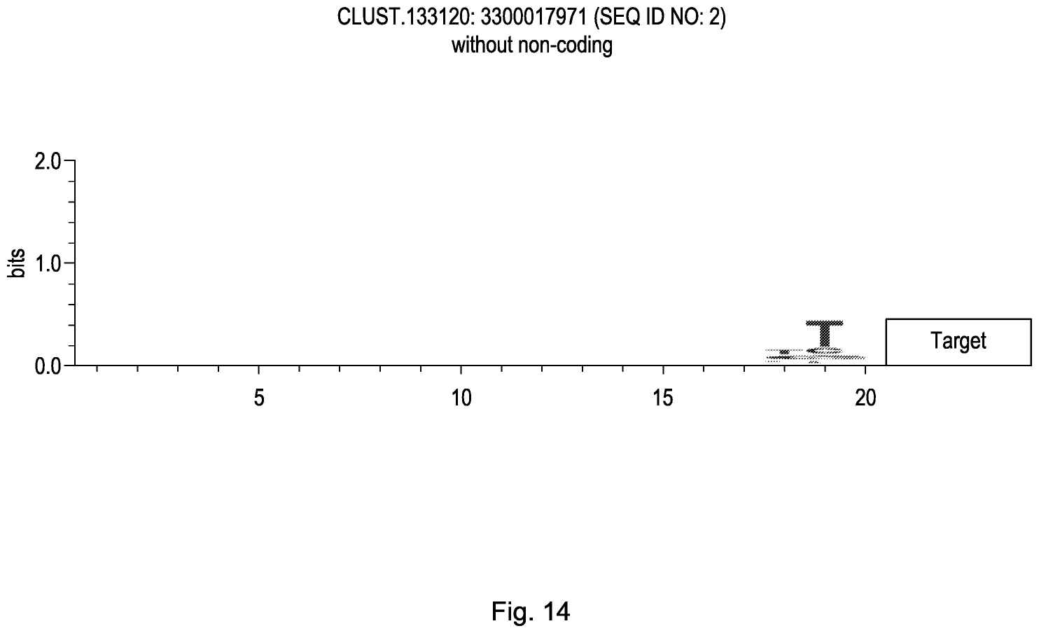

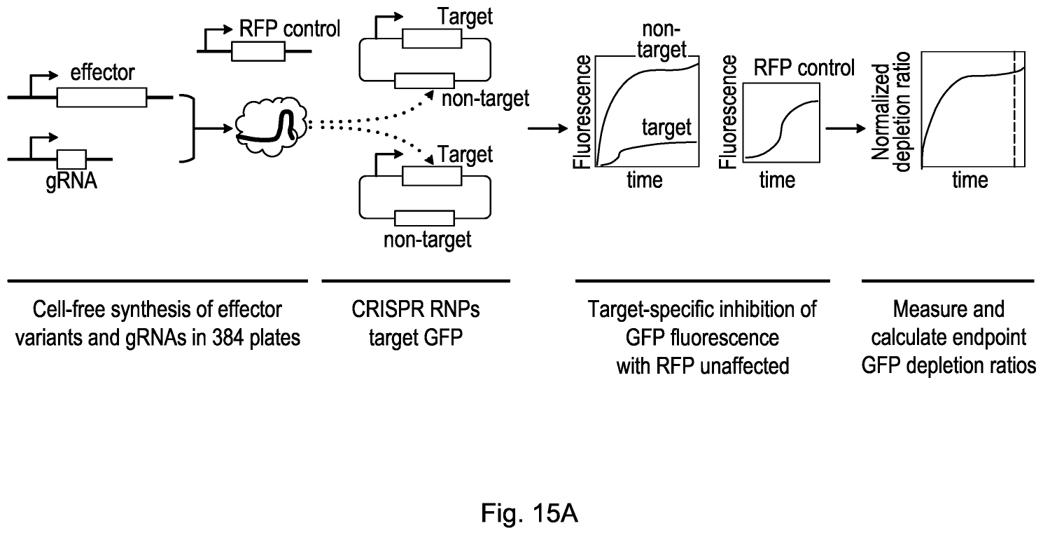

Furthermore, FIG. 9 shows that the CLUST.133120 3300027740 effector retains activity in the absence of the non-coding sequence. In agreement with FIG. 3, the CLUST.133120 3300027740 effector was active in the forward (5′-CCAA . . . CGAC-[spacer]-3′) orientation of the DR. FIG. 10A and FIG. 10B depict the locations of the strongly depleted targets for CLUST.133120 3300027740 effector (minus the non-coding sequence) targeting pACYC184 and E. coli E. Cloni essential genes, respectively. A WebLogo of the PAM sequences for CLUST.133120 3300027740 (minus the non-coding sequence) is shown in FIG. 11. Likewise, FIG. 12 shows that the CLUST.133120 3300017971 effector retains activity in the absence of the non-coding sequence. In agreement with FIG. 6, the CLUST.133120 3300017971 effector was active in the reverse (5′-GGTA . . . CGAC-[spacer]-3′) orientation of the DR. FIG. 13A and FIG. 13B depict the locations of the strongly depleted targets for CLUST.133120 3300017971 effector (minus the non-coding sequence) targeting pACYC184 and E. coli E. Cloni essential genes, respectively. A WebLogo of the PAM sequences for CLUST.133120 3300017971 (minus the non-coding sequence) is shown in FIG. 14. The “20” position corresponds to the nucleotide adjacent to the 5′ location of the target.

As such, multiple effectors of CLUST.133120 CRISPR-Cas show activity in vivo, both in the presence or absence of non-coding sequences. These results suggest that effectors of CLUST.133120 do not require a tracrRNA. CLUST.133120 effectors may thus be self-processing, allowing for ease in multiplexing.

### Example 3—Targeting of GFP by a CLUST.133120 Effector

This Example describes use of a fluorescence depletion assay (FDA) to measure activity of a CLUST.133120 effector.

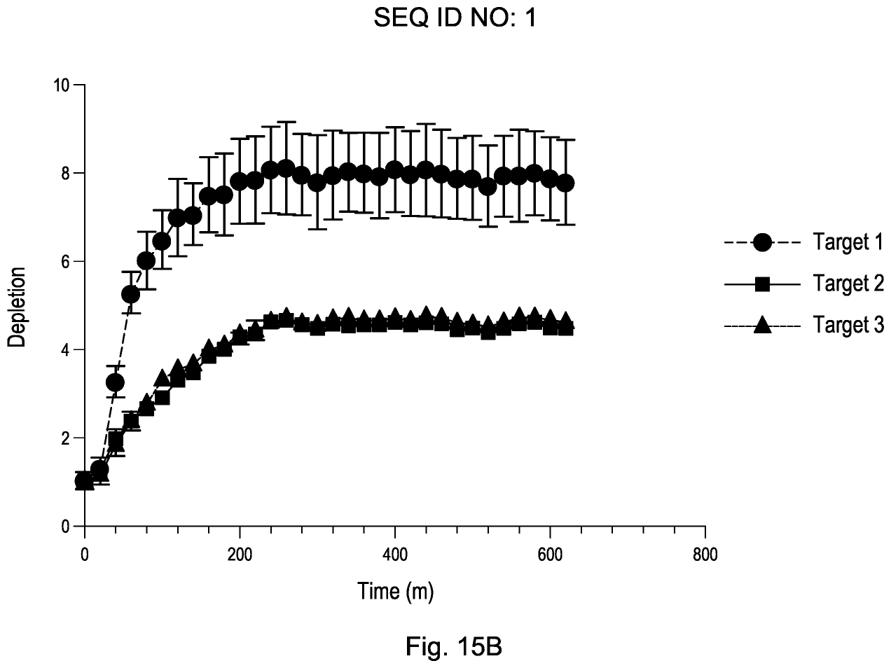

In this assay, an active CRISPR system designed to target GFP binds and cleaves the double-stranded DNA region encoding GFP, resulting in depletion of GFP fluorescence. The FDA assay involves in vitro transcription and translation, allowing production of an RNP from a DNA template encoding a CLUST.133120 effector and a DNA template containing a pre-crRNA sequence under a T7 promoter with direct repeat (DR)-spacer-direct repeat (DR); the spacer targeted GFP. In the same one-pot reaction, GFP and RFP were also produced as both the target and the fluorescence reporter (FIG. 15A). The target GFP plasmid sequence is set forth in SEQ ID NO: 192, and the RFP plasmid sequence is set forth in SEQ ID NO: 193. GFP and RFP fluorescence values were measured every 20 min at 37° C. for 12 hr, using a TECAN Infinite F Plex plate reader. Since RFP was not targeted, its fluorescence was not affected and was therefore used as an internal signal control.

SEQ ID NO: 192
ccccttgtattactgtttatgtaagcagacaggatgcgtc
cggcgtagaggatcgagatctcCAAAAAATGGCTGTTTTT
GAAAAAAATTCTAAAGGTTGTTTTACGACAGACGATAACA
GGGTTgaaataattttgtttaactttaagaaggagATTTA
AATatgAAAATCGAAGAAGGTAAAGGTCACCATCACCATC
ACCACggatccatgacggcattgacggaaggtgcaaaact
gtttgagaaagagatcccgtatatcaccgaactggaaggc
gacgtcgaaggtatgaaatttatcattaaaggcgagggta
ccggtgacgcgaccacgggtaccattaaagcgaaatacat
ctgcactacgggcgacctgccggtcccgtgggcaaccctg
gtgagcaccctgagctacggtgttcagtgtttcgccaagt
acccgagccacatcaaggatttctttaagagcgccatgcc
ggaaggttatacccaagagcgtaccatcagcttcgaaggc
gacggcgtgtacaagacgcgtgctatggttacctacgaac
gcggttctatctacaatcgtgtcacgctgactggtgagaa
ctttaagaaagacggtcacattctgcgtaagaacgttgca
ttccaatgcccgccaagcattctgtatattctgcctgaca
ccgttaacaatggcatccgcgttgagttcaaccaggcgta
cgatattgaaggtgtgaccgaaaaactggttaccaaatgc
agccaaatgaatcgtccgttggcgggctccgcggcagtgc
atatcccgcgttatcatcacattacctaccacaccaaact
gagcaaagaccgcgacgagcgccgtgatcacatgtgtctg
gtagaggtcgtgaaagcggttgatctggacacgtatcagT
AATAAaaagcccgaaaggaagctgagttggctgctgccac
cgctgagcaataactagcataaccccttggggcctctaaa
cgggtcttgaggggttttttgctgaaaggaggaactatat
ccggCTTCCTCGCTCACTGACTCGCTGCGCTCGGTCGTTC
GGCTGCGGCGAGCGGTATCAGCTCACTCAAAGGCGGTAAT
ACGGTTATCCACAGAATCAGGGGATAACGCAGGAAAGAAC
ATGTGAGCAAAAGGCCAGCAAAAGGCCAGGAACCGTAAAA
AGGCCGCGTTGCTGGCGTTTTTCCATAGGCTCCGCCCCCC
TGACGAGCATCACAAAAATCGACGCTCAAGTCAGAGGTGG
CGAAACCCGACAGGACTATAAAGATACCAGGCGTTTCCCC
CTGGAAGCTCCCTCGTGCGCTCTCCTGTTCCGACCCTGCC
GCTTACCGGATACCTGTCCGCCTTTCTCCCTTCGGGAAGC
GTGGCGCTTTCTCATAGCTCACGCTGTAGGTATCTCAGTT
CGGTGTAGGTCGTTCGCTCCAAGCTGGGCTGTGTGCACGA
ACCCCCCGTTCAGCCCGACCGCTGCGCCTTATCCGGTAAC
TATCGTCTTGAGTCCAACCCGGTAAGACACGACTTATCGC
CACTGGCAGCAGCCACTGGTAACAGGATTAGCAGAGCGAG
GTATGTAGGCGGTGCTACAGAGTTCTTGAAGTGGTGGCCT
AACTACGGCTACACTAGAAGAACAGTATTTGGTATCTGCG
CTCTGCTGAAGCCAGTTACCTTCGGAAAAAGAGTTGGTAG
CTCTTGATCCGGCAAACAAACCACCGCTGGTAGCGGTGGT
TTTTTTGTTTGCAAGCAGCAGATTACGCGCAGAAAAAAAG
GATCTCAAGAAGATCCTTTGATCTTTTCTACGGGGTCTGA
CGCTCAGTGGAACGAAAACTCACGggtggcacttttcggg
gaaatgtgcgcggaacccctatttgtttatttttctaaat
acattcaaatatgtatccgctcatgaattaattcttagaa
aaactcatcgagcatcaaatgaaactgcaatttattcata
tcaggattatcaataccatatttttgaaaaagccgtttct
gtaatgaaggagaaaactcaccgaggcagttccataggat
ggcaagatcctggtatcggtctgcgattccgactcgtcca
acatcaatacaacctattaatttcccctcgtcaaaaataa
ggttatcaagtgagaaatcaccatgagtgacgactgaatc
cggtgagaatggcaaaagtttatgcatttctttccagact
tgttcaacaggccagccattacgctcgtcatcaaaatcac
tcgcatcaaccaaaccgttattcattcgtgattgcgcctg
agcgagacgaaatacgcgatcgctgttaaaaggacaatta
caaacaggaatcgaatgcaaccggcgcaggaacactgcca
gcgcatcaacaatattttcacctgaatcaggatattcttc
taatacctggaatgctgttttcccggggatcgcagtggtg
agtaaccatgcatcatcaggagtacggataaaatgcttga
tggtcggaagaggcataaattccgtcagccagtttagtct
gaccatctcatctgtaacatcattggcaacgctacctttg
ccatgtttcagaaacaactctggcgcatcgggcttcccat
acaatcgatagattgtcgcacctgattgcccgacattatc
gcgagcccatttatacccatataaatcagcatccatgttg
gaatttaatcgcggcctagagcaagacgtttcccgttgaa
tatggctcataaca
SEQ ID NO: 193
ccccttgtattactgtttatgtaagcagacaggatgcgtc
cggcgtagaggatcgagatctcCAAAAAATGGCTGTTTTT
GAAAAAAATTCTAAAGGTTGTTTTACGACAGACGATAACA
GGGTTgaaataattttgtttaactttaagaaggagATTTA
AATatgAAAATCGAAGAAGGTAAAGGTCACCATCACCATC
ACCACggatccaTGGTCAGCAAGGGGGAGGAAGACAATAT
GGCTATTATCAAGGAATTCATGCGCTTCAAGGTGCATATG
GAAGGAAGCGTGAATGGACACGAATTCGAGATCGAAGGCG
AGGGGGAGGGTCGCCCTTATGAAGGCACACAAACAGCTAA
ACTGAAAGTGACGAAGGGAGGGCCGCTTCCCTTCGCTTGG
GACATTCTTTCACCCCAGTTCATGTATGGTTCAAAGGCTT
ATGTCAAGCACCCGGCGGACATTCCAGACTACTTAAAATT
GTCGTTCCCCGAGGGGTTTAAATGGGAACGCGTTATGAAT
TTCGAGGATGGGGGAGTCGTAACGGTTACCCAGGACAGTA
GCCTGCAGGATGGCGAGTTCATCTACAAAGTGAAATTGCG
CGGGACGAACTTCCCTAGCGATGGGCCAGTCATGCAGAAG
AAAACGATGGGATGGGAAGCGTCATCCGAGCGCATGTATC
CTGAAGATGGTGCTTTAAAAGGTGAGATCAAGCAGCGTTT
GAAACTGAAGGACGGGGGCCATTATGATGCTGAAGTTAAA
ACGACATATAAGGCCAAGAAGCCAGTTCAACTGCCAGGGG
CTTATAATGTTAATATTAAATTAGACATTACGAGCCATAA
TGAAGATTACACGATTGTCGAGCAATACGAGCGCGCAGAA
GGACGCCACTCAACGGGGGGCATGGACGAGCTGTACAAGT
AAaaagcccgaaaggaagctgagttggctgctgccaccgc
tgagcaataactagcataaccccttggggcctctaaacgg
gtcttgaggggttttttgctgaaaggaggaactatatccg
gCTTCCTCGCTCACTGACTCGCTGCGCTCGGTCGTTCGGC
TGCGGCGAGCGGTATCAGCTCACTCAAAGGCGGTAATACG
GTTATCCACAGAATCAGGGGATAACGCAGGAAAGAACATG
TGAGCAAAAGGCCAGCAAAAGGCCAGGAACCGTAAAAAGG
CCGCGTTGCTGGCGTTTTTCCATAGGCTCCGCCCCCCTGA
CGAGCATCACAAAAATCGACGCTCAAGTCAGAGGTGGCGA
AACCCGACAGGACTATAAAGATACCAGGCGTTTCCCCCTG
GAAGCTCCCTCGTGCGCTCTCCTGTTCCGACCCTGCCGCT
TACCGGATACCTGTCCGCCTTTCTCCCTTCGGGAAGCGTG
GCGCTTTCTCATAGCTCACGCTGTAGGTATCTCAGTTCGG
TGTAGGTCGTTCGCTCCAAGCTGGGCTGTGTGCACGAACC
CCCCGTTCAGCCCGACCGCTGCGCCTTATCCGGTAACTAT
CGTCTTGAGTCCAACCCGGTAAGACACGACTTATCGCCAC
TGGCAGCAGCCACTGGTAACAGGATTAGCAGAGCGAGGTA
TGTAGGCGGTGCTACAGAGTTCTTGAAGTGGTGGCCTAAC
TACGGCTACACTAGAAGAACAGTATTTGGTATCTGCGCTC
TGCTGAAGCCAGTTACCTTCGGAAAAAGAGTTGGTAGCTC
TTGATCCGGCAAACAAACCACCGCTGGTAGCGGTGGTTTT
TTTGTTTGCAAGCAGCAGATTACGCGCAGAAAAAAAGGAT
CTCAAGAAGATCCTTTGATCTTTTCTACGGGGTCTGACGC
TCAGTGGAACGAAAACTCACGggtggcacttttcggggaa
atgtgcgcggaacccctatttgtttatttttctaaataca
ttcaaatatgtatccgctcatgaattaattcttagaaaaa
ctcatcgagcatcaaatgaaactgcaatttattcatatca
ggattatcaataccatatttttgaaaaagccgtttctgta
atgaaggagaaaactcaccgaggcagttccataggatggc
aagatcctggtatcggtctgcgattccgactcgtccaaca
tcaatacaacctattaatttcccctcgtcaaaaataaggt
tatcaagtgagaaatcaccatgagtgacgactgaatccgg
tgagaatggcaaaagtttatgcatttctttccagacttgt
tcaacaggccagccattacgctcgtcatcaaaatcactcg
catcaaccaaaccgttattcattcgtgattgcgcctgagc
gagacgaaatacgcgatcgctgttaaaaggacaattacaa
acaggaatcgaatgcaaccggcgcaggaacactgccagcg
catcaacaatattttcacctgaatcaggatattcttctaa
tacctggaatgctgttttcccggggatcgcagtggtgagt
aaccatgcatcatcaggagtacggataaaatgcttgatgg
tcggaagaggcataaattccgtcagccagtttagtctgac
catctcatctgtaacatcattggcaacgctacctttgcca
tgtttcagaaacaactctggcgcatcgggcttcccataca
atcgatagattgtcgcacctgattgcccgacattatcgcg
agcccatttatacccatataaatcagcatccatgttggaa
tttaatcgcggcctagagcaagacgtttcccgttgaatat
ggctcataaca

3 GFP targets (plus 1 non-target) were designed for the effector of SEQ ID NO: 1. RNA guide sequences, target sequences, and the non-target control sequences used for the FDA assay are listed in Table 6. The pre-crRNA sequences shown in Table 6 further include a T7 promoter at the 5′ end and a hairpin motif that caps the 3′ end of the RNA to ensure that the RNA is not degraded by nucleases present in the in vitro transcription and translation mixture. A 5′-TTN-3′ PAM was used for the target sequences.

TABLE 6
RNA guide and Target Sequences for FDA Assay.

| Target | pre-crRNA Sequence | Target Sequence |
|---|---|---|
| Target 1 | CCAACCAATGCC | tcgtctgtcgtaaaac |
| | AGCGCGACGGCT | aacctttagaattttt |
| | TATGAGTCGCGA | ttc |
| | Ctcgtctgtcgt | (SEQ ID |
| | aaaacaaccttt | NO: 82) |
| | agaatttttttc | |
| | CCAACCAATGCC | |
| | AGCGCGACGGCT | |
| | TATGAGTCGCGA | |
| | C | |
| | (SEQ ID | |
| | NO: 916) | |
| Target 2 | gaaattaatacg | cagttcggtgat |
| | actcactatagg | atacgggatctc |
| | gCCAACCAATGC | tttctcaaaca |
| | CAGCGCGACGGC | (SEQ ID |
| | TTATGAGTCGCG | NO: 83) |
| | ACcagttcggtg | |
| | atatacgggatc | |
| | tctttctcaaac | |
| | aCCAACCAATGC | |
| | CAGCGCGACGGC | |
| | TTATGAGTCGCG | |
| | AC | |
| | (SEQ ID | |
| | NO: 917) | |
| Target 3 | gaaattaatacg | ggtgatatacgg |
| | actcactatagg | gatctctttctc |
| | gCCAACCAATGC | aaacagttttg |
| | CAGCGCGACGGC | (SEQ ID |
| | TTATGAGTCGCG | NO: 84) |
| | ACggtgatatac | |
| | gggatctctttc | |
| | tcaaacagtttt | |
| | gCCAACCAATGC | |
| | CAGCGCGACGGC | |
| | TTATGAGTCGCG | |
| | AC | |
| | (SEQ ID | |
| | NO: 918) | |
| Non-Target 4 | agaaattaatac | |
| | gactcactatag | |
| | ggCCAACCAATG | |
| | CCAGCGCGACGG | |
| | CTTATGAGTCGC | |
| | GACaggtgctac | |
| | atttgaagagat | |
| | aaattgcactga | |
| | aaCCAACCAATG | |
| | CCAGCGCGACGG | |
| | CTTATGAGTCGC | |
| | GACtaacccctc | |
| | tctaaacggagg | |
| | ggttt | |
| | (SEQ ID | |
| | NO: 919) | |

GFP signal was normalized to RFP signal, then the average fluorescence of three technical replicates was taken at each time point. GFP fluorescence depletion was then calculated by dividing the GFP signal of an effector incubated with a non-GFP targeting RNA guide (which instead targets a kanamycin resistance gene and does not deplete GFP signal) by the GFP signal of an effector incubated with a GFP targeting RNA guide. The resulting value is referred to as “Depletion” in FIG. 15B.

A Depletion of one or approximately one indicated that there was little to no difference in GFP depletion with respect to a non-GFP targeting pre-crRNA and a GFP targeting pre-crRNA (e.g., 10 RFU/10 RFU=1). A Depletion of greater than one indicated that there was a difference in GFP depletion with respect to a non-GFP targeting pre-crRNA and a GFP targeting pre-crRNA (e.g., 10 RFU/5 RFU=2). Depletion of the GFP signal indicated that the effector formed a functional RNP and interfered with the production of GFP by introducing double-stranded DNA cleavage within the GFP coding region. The extent of the GFP depletion was largely correlated to the specific activity of a CLUST.133120 effector.

FIG. 15B shows depletion curves for RNPs formed by the effector of SEQ ID NO: 1, measured every 20 minutes for each of the GFP targets (Targets 1-3). At each target, the depletion values for RNPs formed with the effector of SEQ ID NO: 1 were greater than one.

This indicated that the CLUST.133120 effector formed a functional RNP capable of interfering with the production of GFP.

### Example 4-Identification of Components of CLUST.099129 CRISPR-Cas System

This protein family was identified using the computational methods described above. The CLUST.099129 system comprises single effectors associated with CRISPR systems found in uncultured metagenomic sequences collected from freshwater, wastewater, soil, and rhizosphere environments as well as from Anaerolineae bacterium (TABLE 9). Exemplary CLUST.099129 effectors include those shown in TABLES 9 and 10, below. Examples of direct repeat sequences for these systems are shown in TABLE 11. Optionally, the system includes a tracrRNA that is contained in a non-coding sequence listed in TABLE 12.

TABLE 9
Representative CLUST.099129 Effector Proteins

| | | # | Effector | SEQ |
|---|---|---|---|---|
| Source | Effector Accession | Spacers | Size | ID NO |
| wastewater metagenome | SRR6837557_1470862_2\|M | 3 | 541 | 101 |
| terrestrial-soil-tropical | 3300012971\|Ga0126369_10127246_1\|M | 7 | 586 | 102 |
| forest soil | | | | |
| terrestrial-soil-tropical | 3300005764\|Ga0066903_100343966_1\|M | 6 | 600 | 103 |
| forest soil | | | | |
| Anaerolineae bacterium | PMDR01000701_10\|P | 8 | 522 | 104 |
| aquatic-freshwater-bog | 3300014158\|Ga0181521_10019591_3\|P | 2 | 520 | 105 |
| aquatic-freshwater-bog | 3300014159\|Ga0181530_10005765_11\|M | 2 | 520 | 106 |
| aquatic-freshwater-bog | 3300014638\|Ga0181536_10002122_15\|P | 2 | 520 | 107 |
| aquatic-freshwater- | 3300009632\|Ga0116102_1009001_2\|P | 5 | 520 | 108 |
| peatland | | | | |
| aquatic-freshwater- | 3300009643\|Ga0116110_1007258_2\|M | 5 | 534 | 109 |
| peatland | | | | |
| aquatic-freshwater- | 3300009643\|Ga0116110_1007258_2\|P | 5 | 520 | 110 |
| peatland | | | | |
| aquatic-freshwater- | 3300017996\|Ga0187891_1001667_23\|M | 2 | 520 | 111 |
| peatland | | | | |
| aquatic-freshwater- | 3300018003\|Ga0187876_1014226_2\|P | 2 | 520 | 112 |
| peatland | | | | |
| aquatic-freshwater- | 3300025453\|Ga0208455_1001030_8\|P | 5 | 520 | 113 |
| peatland | | | | |
| aquatic-freshwater- | 3300025507\|Ga0208188_1002252_2\|M | 5 | 534 | 114 |
| peatland | | | | |
| aquatic-freshwater- | 3300025507\|Ga0208188_1002252_2\|P | 5 | 520 | 115 |
| peatland | | | | |
| biofilm metagenome | PJQE01000204_1\|M | 2 | 526 | 116 |
| plants-rhizosphere- | 3300006845\|Ga0075421_100006223_8\|P | 2 | 586 | 117 |
| populus rhizosphere | | | | |
| plants-rhizosphere- | 3300006847\|Ga0075431_100017297_1\|M | 2 | 586 | 118 |
| populus rhizosphere | | | | |
| plants-rhizosphere- | 3300027909\|Ga0209382_10008589_8\|P | 2 | 586 | 119 |
| populus rhizosphere | | | | |
| soil metagenome | ODAK010035847_2\|M | 4 | 506 | 120 |
| soil metagenome | SRR6266511_1572288_18\|M | 4 | 615 | 121 |
| soil metagenome | SRR6266511_203987_6\|P | 5 | 673 | 122 |
| soil metagenome | SRR6266511_239355_1\|M | 17 | 602 | 123 |
| soil metagenome | SRR6266511_838544_1\|M | 7 | 600 | 124 |
| terrestrial-soil-fen | 3300014502\|Ga0182021_10233972_2\|M | 3 | 522 | 125 |
| terrestrial-soil-surface | 3300005529\|Ga0070741_10001368_72\|P | 2 | 547 | 126 |
| soil | | | | |
| terrestrial-soil-tropical | 3300010046\|Ga0126384_10047357_3\|M | 5 | 615 | 127 |
| forest soil | | | | |
| terrestrial-soil-tropical | 3300010359\|Ga0126376_10030051_2\|M | 5 | 615 | 128 |
| forest soil | | | | |
| terrestrial-soil-tropical | 3300010362\|Ga0126377_10111069_2\|M | 4 | 615 | 129 |
| forest soil | | | | |
| terrestrial-soil-vadose | 3300012204\|Ga0137374_10002290_4\|M | 5 | 673 | 130 |
| zone soil | | | | |
| terrestrial-soil-vadose | 3300012204\|Ga0137374_10009879_6\|P | 4 | 615 | 131 |
| zone soil | | | | |
| terrestrial-soil-vadose | 3300012350\|Ga0137372_10022207_3\|M | 4 | 615 | 132 |
| zone soil | | | | |
| terrestrial-soil-vadose | 3300012350\|Ga0137372_10022207_4\|P | 4 | 614 | 133 |
| zone soil | | | | |
| terrestrial-soil-vadose | 3300012353\|Ga0137367_10000906_4\|M | 5 | 673 | 134 |
| zone soil | | | | |
| terrestrial-soil-vadose | 3300012353\|Ga0137367_10004593_5\|M | 4 | 615 | 135 |
| zone soil | | | | |
| terrestrial-soil-vadose | 3300012355\|Ga0137369_10001944_2\|P | 5 | 673 | 136 |
| zone soil | | | | |
| terrestrial-soil-vadose | 3300012355\|Ga0137369_10007378_6\|M | 4 | 615 | 137 |
| zone soil | | | | |
| terrestrial-soil-vadose | 3300012355\|Ga0137369_10043960_1\|M | 8 | 602 | 138 |
| zone soil | | | | |
| terrestrial-soil-vadose | 3300012358\|Ga0137368_10001884_4\|M | 5 | 673 | 139 |
| zone soil | | | | |
| terrestrial-soil-vadose | 3300012358\|Ga0137368_10007571_6\|P | 4 | 615 | 140 |
| zone soil | | | | |
| terrestrial-soil-vadose | 3300012360\|Ga0137375_10064112_1\|P | 5 | 673 | 141 |
| zone soil | | | | |
| terrestrial-soil-vadose | 3300012532\|Ga0137373_10033017_1\|P | 5 | 673 | 142 |
| zone soil | | | | |
| terrestrial-soil-vadose | 3300012944\|Ga0137410_10000075_50\|P | 2 | 548 | 143 |
| zone soil | | | | |
| terrestrial-soil-vadose | 3300015245\|Ga0137409_10000164_50\|P | 2 | 548 | 144 |
| zone soil | | | | |
| wastewater metagenome | SRR6837571_912213_1\|M | 3 | 541 | 145 |

TABLE 10
Amino acid sequences of Representative
CLUST.099129 Effector Proteins

>SRR6837557_14708622|M
[wastewater metagenome]
MRTTKTPDYSNKNLSFWCEPVGEMPSVVFEKGRQMNRLWN
LLVECHTTFLAKHDGKKGGERTEAYKTEWQPLLKRIYNNE
AKSFGLNSDETNFVTDGFKNAIRAGYDQKRPGAGMPTFKA
RLRSIHIPFCDRNGGRAPAWFFEKPDRRIHLRPILLPDAA
KRVIDRGPARGHFTVDGQAISLRANLSQSIPPGALVKRVS
LVGKLERPFGWQWKLMFSIAYPPPPPAQSDAACVGVDLGY
RRFEDRIRFGMIFDGSRFHELALPLDLTNRRAAKAGKRWD
IREIWELESQIGCGVEACKASLKQLPKTDWPEEARGAMQG
IAKMRERGLLKVRRLLRDAEITVPELETWWDDYQRLFRKS
RHLELQIVATRTHLYRNLALSLARSCAAIAWEGDLSLRDL
AESAGKKKKQRKEAIAEGVSAERSPEDRIEEAAQKWRAYV
CLSDFRRFLKEAVEKTTAALIDRPAAGTTNTCEECGAPVD
TGPDLVLTCANGHRRDQDKGSAKMLFEEIDEGYRQQSAPL
SLDLFTDQAVRAIRTLNREMV
(SEQ ID NO: 101)
>3300012971|Ga0126369_10127246_1|M
[terrestrial-soil-tropical forest soil]
MIEEKTAKRKNPVRRERLNKDWPIKVYTYDCWPQEKPLKA
LWDTAHEMRRLWNDFTAIFRKILTDDKKDEASRPLLSKEE
RAILWAPINTKPLREIAKQRKKRLDAGCREAVVVRFLTTV
GNWRKNPSRCGPPRFQRADEMNAISIPLVYNDGKSAEWLI
GGEGTSSVRETRNLKTEAPEEYLNNGHFCVGVTRERLNLH
IAYGGRSGRKKYYLPEGCRIKQVTLCGRRDSAFNWSWSFQ
VRLEHPPEPARQPTGRVCGWDSCGWRLMDGYIRLGVIADN
AGHFYELMVPLAIGAACRRMRREKEYCLKQGWEYGKPLSF
DDAEALDARYGTTLEACKTQVRKIYEGEKENWPAGARKIM
SGIMKMRDTGLRRLRRKLEPIESEAKRTIDDWDAEAAKMN
ETIRAFEIHANNAKRDAYRQIAAWLNAFDIIAWEGDLSLK
QMAEKAGKKKQKRKEAHEETGQWNERTPEERQFEASQKYR
QIAGQRQLREFVKQKHESRLQNEKAAYSTRTCPECGGTIE
PGRKLLLVCENGHVRDQDVSAALHFLNKIEGVASITAPPV
EIPAHLGPYLRVMDASEVRLELAEKR
(SEQ ID NO: 102)
>3300005764|Ga0066903_100343966_1|M
[terrestrial-soil-tropical forest soil]
MEDISKKPERRKNPARRERLNKDWPIKVHNYRCSPNEKPP
QALWDTAHEMRRLWNDLTAVFRKILAEDKKDEDGKPLLSK
EERANLWAPINVKPLREIAKQRKDKLDIGCREYVVTHFIT
TVNNWRRNPRRFGPPRFQNAADMDSVHIPLVYTDGKSADW
LTGGNGTAWVRDTLHIKKNALRDKDGCPVLSENELEEYLN
NGHFCVGITREKLSLHIAYGGRSGRKKYHLPDKCRIKQVA
LCGRRNSAFGWSWSFQVRLEHPPDPERQVTGRVCGWDSGG
WRKMDSYIRLGVIADNAGHFYELMVPLAIGAASHRLRHER
EHCQKNGWEYNKPITFDDAEALQSRYGLALEACKTNLRII
FEEEKEKWPDDARKIMGGIIRMRDDGLRRLRRKLDEIESA
AKQTIDDWDAEAAKLNETIRAFQRHAGNAKRNAYRQITAW
LSAFDRIAWEGDLNLKWMAEQPGKKKQERKKTLAETGQWG
ERTAEDRQLEASQRYRQIVGQYRLRELVKQTHEARLQNEK
TAHSTRTCSECGARINPGGKLMLVCEKGHKRDQDVTASLY
LLSKIEGYASISGPPIEIPAHLSPYLRVMTASDVALEIVE
(SEQ ID NO: 103)
>PMDR01000701_10|P
[Anaerolineae bacterium]
MRPKKNADTIYQTYKFWIKPSDPLPQLLWDKAKAMNAAWT
SLVLMRQETLETWMAQRDTLTADQKRAFWNTFNLAVREKI
EECELDWETSGVLHDRFLVAHQRALKDKADLHGRTRLQRI
LLVHRYTGGGIPIEKLLSTRAWRFALDAFPTEDVYATNTR
QARRGRQTQGRFGLDNETAVEFSIILHRPLPPAAIVKRVA
LSGRRQKPPAHEWDWALVVSVEVPNPSRTAAGRVIGIDLG
WRKIDDYLRIGYAADDTDQQIELRLPLDFSTQKARSAGYP
CNHAELRALEGWIAVAVDATK
AAIGELITVPSLVLMRQGGLVRLQRTLSEKPDLSEAEVEA
LRILGAWRKQNDRLQKIYNFSTARFAARRRWLYQNLAAWL
CRNYDVIAIEGDLDVKGLAEEPDPDPALKAAQRYRQIAAP
GELREIIRWTAQREGVIVKDCNTAYSTTTCFTCGEQAEAG
PDLILECPAGHRFDQDANAAANLLSQILPLTEVQDGLRKN
QRDFLRIPPELQMVAWVHRD
(SEQ ID NO: 104)
>3300014158|Ga018152l_10019591_3|P
[aquatic-freshwater-bog]
MQRERKRKESHDIRPHKYRAWFAEGDGTRLPEPVYLLAKR
MQDCWTDIALHDQQAYDEWRTAHPVPTPPAGLEGEALAKW
RKENWPKPPKPFYVGNQEWAEGRVGQANLPDELGQTILDR
LAVTFKNMKKGGGPPKPHGRLDKFTFHHRYTGGGLPVNKL
GRQLGERFRISIPPPQAYQPKEDIMNPRASRCQRVCLAWF
RVDGVPVRLNVMMHRSLPEGDVYVKRVALVGRKSSAALPW
EVYLVFTLEVPSETEAQAPLGTRCGIDVGWRKLDDGRMRV
AVLHDGKPTPEELIMPLRIYDRKLGEVSLERLANLKRCRD
GLLERTKATVAAMFGTPLSDWTRIRNGGLIRLMREDGAPA
EVIAMLETWKRDSDRYLRMERMLENRMRRHYEWRYGKWAK
DVATRHRTVRIKKMDQKEMWAVEKTKDYPALKASAERRKL
AAGGSLLAMVRHAVRKACGNLVEVEAAHTTDTCAACGSHF
EAGAGLMGRCEQGHVMDQDWNAAGNIYAGRAGATAQAAVG
(SEQ ID NO: 105)
>3300014159|Ga0181530_10005765_11|M
[aquatic-freshwater-bog]
MQRERKRKESHDIRPHKYRAWFAEGDGTRLPEPVYLLAKR
MQDCWTDIALHDQQAYDEWRTAHPVPTPPAGLEGEALAKW
RKENWPKPPKPFYVGNQEWAEGRVGQANLPDELGQTILDR
LAVTFKNMKKGGGPPKPHGRLDKFTFHHRYTGGGLPVNKL
GRQLGERFRISIPPPQAYQPKEDIMNPRASRCQRVCLAWF
RVDGVPVRLNVMMHRSLPEGDVYVKRVALVGRKSSAALPW
EVYLVFTLEVPSETEAQAPLGTRCGIDVGWRKLDDGRMRV
AVLHDGKPTPEELIMPLRIYDRKLGEVSLERLANLKRCRD
GLLERTKATVAAMFGTPLSDWTRIRNGGLIRLMREDGAPA
EVIAMLETWKRDSDRYLRMERMLENRMRRHYEWRYGKWAK
DVATRHRTVRIKKMDQKEMWAVEKTKDYPALKASAERRKL
AAGGSLLAMVRHAVRKACGNLVEVEAAHTTDTCAACGSHF
EAGAGLMGRCEQGHVMDQDWNAAGNIYAGRAGATAQAAVG
(SEQ ID NO: 106)
>3300014638|Ga0181536_10002122_15|P
[aquatic-freshwater-bog]
MQRERKRKESHDIRPHKYRAWFAEGDGTRLPEPVYLLAKR
MQDCWTDIALHDQQAYDEWRTAHPVPTPPAGLEGEALAKW
RKENWPKPPKPFYVGNQEWAEGRVGQANLPDELGQTILDR
LAVTFKNMKKGGGPPKPHGRLDKFTFHHRYTGGGLPVNKL
GRQLGERFRISIPPPQAYQPKEDIMNPRASRCQRVCLAWF
RVDGVPVRLNVMMHRSLPEGDVYVKRVALVGRKSSAALPW
EVYLVFTLEVPSETEAQAPLGTRCGIDVGWRKLDDGRMRV
AVLHDGKPTPEELIMPLRIYDRKLGEVSLERLANLKRCRD
GLLERTKATVAAMFGTPLSDWTRIRNGGLIRLMREDGAPA
EVIAMLETWKRDSDRYLRMERMLENRMRRHYEWRYGKWAK
DVATRHRTVRIKKMDQKEMWAVEKTKDYPALKASAERRKL
AAGGSLLAMVRHAVRKACGNLVEVEAAHTTDTCAACGSHF
EAGAGLMGRCEQGHVMDQDWNAAGNIYAGRAGATAQAAVG
(SEQ ID NO: 107)
>3300009632|Ga0116102_1009001_2|P
[aquatic-freshwater-peatland]
MQRERKRKETHDIRPHKYRAWFAEGDGSRLPEPVFALARR
MQDCWTDIALHNQDAYDEWRMAHPAPAAPAGLEGQTIEKW
RRENWPKLPKPYYEGNQAWAESRVRQANLPGELGQTILDR
LSVTFKNMKKGGGPPKPHYRLDRFAFHHRYTSGGLPVLGL
RSQRSQRFHILLPSPHAYQTKGGTKNPRELRRQRVCPAWF
CVDGVSARLNVMMHRPLPEGDAYVKRVALVGKKSSAAMPW
EVSLALTVEVPVAKEPQAPAGTRCGLDVGWRKLDKDRMRV
AVLYNGGPNSEELVMPLRIHDRKLGEVSLERLARLNRCRD
ALLERTKAAVATMLGTEPSAWAQMRNGGLVRLMREDGAPP
EVIEVLEAWKQDNDRYLRLERLVEAHLRRHYEWRYGKWAK
DVATRYRTVRIRKMGQKEMWAVEKTKDYPALKASAERRKL
AAGGSLLSMVRHAVRKACGNLVEVEAAHTTDTCAACGSHF
EAGAGLMGRCEQGHVMDQDWNAAGNIYAGRAGATAQAAVG
(SEQ ID NO: 108)
>3300009643|Ga0116110_1007258_2|M
[aquatic-freshwater-peatland]
MSARTIEERKGKTSMQRERKRKETHDIRPHKYRAWFAEGD
GTRLPEPIFALAKRMQDCWTDIALHNQDAFEEWRMAHPLP
APPDGLAGEAFEDWRRKNWPRPPKPFYEANQAWAESRVRQ
ANLPGELGETILDRLSVTVNNMKKGGGPPKPHYRLDRFAF
HHRYTSGGLPVLGLRSQRSQRFHILLPPPNAYQPKGGMRN
PHELRRQRVCPAWFCVDGVPVRLNVMMHRPLPEGDAYLKR
VALVGKKSSAALPWEVFLVLTVEVPLAKEPQAPAGTRCGL
DVGWRKLDKDRMRVAVLYNGGPNSEELVMPLRIHDRKLGE
VSLERLARLNRCRDALLERTKAAVATMLGTEPSAWAQMRN
GGLVRLMREDGAPPEVIEVLEAWKQDNDRYLRLERLVEAH
LRRHYEWRYGKWAKDVATRYRTVRIRKMGQKEMWAVEKTK
DYPALKASAERRKLAAGGSLLSMVRHAVRKACGNLVEVEA
AHTTDTCAACGSHFEAGAGLMGRCEQGHVMDQDWNAAGNI
YAGRAGATAQAAVG
(SEQ ID NO: 109)
>3300009643|Ga0116110_1007258_2|P
[aquatic-freshwater-peatland]
MQRERKRKETHDIRPHKYRAWFAEGDGTRLPEPIFALAKR
MQDCWTDIALHNQDAFEEWRMAHPLPAPPDGLAGEAFEDW
RRKNWPRPPKPFYEANQAWAESRVRQANLPGELGETILDR
LSVTVNNMKKGGGPPKPHYRLDRFAFHHRYTSGGLPVLGL
RSQRSQRFHILLPPPNAYQPKGGMRNPHELRRQRVCPAWF
CVDGVPVRLNVMMHRPLPEGDAYLKRVALVGKKSSAALPW
EVFLVLTVEVPLAKEPQAPAGTRCGLDVGWRKLDKDRMRV
AVLYNGGPNSEELVMPLRIHDRKLGEVSLERLARLNRCRD
ALLERTKAAVATMLGTEPSAWAQMRNGGLVRLMREDGAPP
EVIEVLEAWKQDNDRYLRLERLVEAHLRRHYEWRYGKWAK
DVATRYRTVRIRKMGQKEMWAVEKTKDYPALKASAERRKL
AAGGSLLSMVRHAVRKACGNLVEVEAAHTTDTCAACGSHF
EAGAGLMGRCEQGHVMDQDWNAAGNIYAGRAGATAQAAVG
(SEQ ID NO: 110)
>3300017996|Ga0187891_1001667_23|M
[aquatic-freshwater-peatland]
MQRERKRKESHDIRPHKYRAWFAEGDGTRLPEPVYLLAKR
MQDCWTDIALHDQQAYDEWRTAHPVPTPPAGLEGEALAKW
RKENWPKPPKPFYVGNQEWAEGRVGQANLPDELGQTILDR
LAVTFKNMKKGGGPPKPHGRLDKFTFHHRYTGGGLPVNKL
GRQLGERFRISIPPPQAYQPKEDIMNPRASRCQRVCLAWF
RVDGVPVRLNVMMHRSLPEGDVYVKRVALVGRKSSAALPW
EVYLVFTLEVPSETEAQAPLGTRCGIDVGWRKLDDGRMRV
AVLHDGKPTPEELIMPLRIYDRKLGEVSLERLANLKRCRD
GLLERTKATVAAMFGTPLSDWTRIRNGGLIRLMREDGAPA
EVIAMLETWKRDSDRYLRMERMLENRMRRHYEWRYGKWAK
DVATRHRTVRIKKMDQKEMWAVEKTKDYPALKASAERRKL
AAGGSLLAMVRHAVRKACGNLVEVEAAHTTDTCAACGSHF
EAGAGLMGRCEQGHVMDQDWNAAGNIYAGRAGATAQAAVG
(SEQ ID NO: 111)
>3300018003|Ga0187876_1014226_2|P
[aquatic-freshwater-peatland]
MQRERKRKESHDIRPHKYRAWFAEGDGTRLPEPVYLLAKR
MQDCWTDIALHDQQAYDEWRTAHPVPTPPAGLEGEALAKW
RKENWPKPPKPFYVGNQEWAEGRVGQANLPDELGQTILDR
LAVTFKNMKKGGGPPKPHGRLDKFTFHHRYTGGGLPVNKL
GRQLGERFRISIPPPQAYQPKEDIMNPRASRCQRVCLAWF
RVDGVPVRLNVMMHRSLPEGDVYVKRVALVGRKSSAALPW
EVYLVFTLEVPSETEAQAPLGTRCGIDVGWRKLDDGRMRV
AVLHDGKPTPEELIMPLRIYDRKLGEVSLERLANLKRCRD
GLLERTKATVAAMFGTPLSDWTRIRNGGLIRLMREDGAPA
EVIAMLETWKRDSDRYLRMERMLENRMRRHYEWRYGKWAK
DVATRHRTVRIKKMDQKEMWAVEKTKDYPALKASAERRKL
AAGGSLLAMVRHAVRKACGNLVEVEAAHTTDTCAACGSHF
EAGAGLMGRCEQGHVMDQDWNAAGNIYAGRAGATAQAAVG
(SEQ ID NO: 112)
>3300025453|Ga0208455_1001030_8|P
[aquatic-freshwater-peatland]
MQRERKRKETHDIRPHKYRAWFAEGDGSRLPEPVFALARR
MQDCWTDIALHNQDAYDEWRMAHPAPAAPAGLEGQTIEKW
RRENWPKLPKPYYEGNQAWAESRVRQANLPGELGQTILDR
LSVTFKNMKKGGGPPKPHYRLDRFAFHHRYTSGGLPVLGL
RSQRSQRFHILLPSPHAYQTKGGTKNPRELRRQRVCPAWF
CVDGVSARLNVMMHRPLPEGDAYVKRVALVGKKSSAAMPW
EVSLALTVEVPVAKEPQAPAGTRCGLDVGWRKLDKDRMRV
AVLYNGGPNSEELVMPLRIHDRKLGEVSLERLARLNRCRD
ALLERTKAAVATMLGTEPSAWAQMRNGGLVRLMREDGAPP
EVIEVLEAWKQDNDRYLRLERLVEAHLRRHYEWRYGKWAK
DVATRYRTVRIRKMGQKEMWAVEKTKDYPALKASAERRKL
AAGGSLLSMVRHAVRKACGNLVEVEAAHTTDTCAACGSHF
EAGAGLMGRCEQGHVMDQDWNAAGNIYAGRAGATAQAAVG
(SEQ ID NO: 113)
>3300025507|Ga0208188_1002252_2|M
[aquatic-freshwater-peatland]
MSARTIEERKGKTSMQRERKRKETHDIRPHKYRAWFAEGD
GTRLPEPIFALAKRMQDCWTDIALHNQDAFEEWRMAHPLP
APPDGLAGEAFEDWRRKNWPRPPKPFYEANQAWAESRVRQ
ANLPGELGETILDRLSVTVNNMKKGGGPPKPHYRLDRFAF
HHRYTSGGLPVLGLRSQRSQRFHILLPPPNAYQPKGGMRN
PHELRRQRACPAWFCVDGVPVRLNVMMHRPLPEGDAYLKR
VALVGKKSSAALPWEVFLVLTVEVPLAKEPQAPAGTRCGL
DVGWRKLDKDRMRVAVLYNGGPNSEELVMPLRIHDRKLGE
VSLERLARLNRCRDALLERTKAAVATMLGTEPSAWAQMRN
GGLVRLMREDGAPPEVIEVLEAWKQDNDRYLRLERLVEAH
LRRHYEWRYGKWAKDVATRYRTVRIRKMGQKEMWAVEKTK
DYPALKASAERRKLAAGGSLLSMVRHAVRKACGNLVEVEA
AHTTDTCAACGSHFEAGAGLMGRCEQGHVMDQDWNAAGNI
YAGRAGATAQAAVG
(SEQ ID NO: 114)
>3300025507|Ga0208188_1002252_2|P
[aquatic-freshwater-peatland]
MQRERKRKETHDIRPHKYRAWFAEGDGTRLPEPIFALAKR
MQDCWTDIALHNQDAFEEWRMAHPLPAPPDGLAGEAFEDW
RRKNWPRPPKPFYEANQAWAESRVRQANLPGELGETILDR
LSVTVNNMKKGGGPPKPHYRLDRFAFHHRYTSGGLPVLGL
RSQRSQRFHILLPPPNAYQPKGGMRNPHELRRQRACPAWF
CVDGVPVRLNVMMHRPLPEGDAYLKRVALVGKKSSAALPW
EVFLVLTVEVPLAKEPQAPAGTRCGLDVGWRKLDKDRMRV
AVLYNGGPNSEELVMPLRIHDRKLGEVSLERLARLNRCRD
ALLERTKAAVATMLGTEPSAWAQMRNGGLVRLMREDGAPP
EVIEVLEAWKQDNDRYLRLERLVEAHLRRHYEWRYGKWAK
DVATRYRTVRIRKMGQKEMWAVEKTKDYPALKASAERRKL
AAGGSLLSMVRHAVRKACGNLVEVEAAHTTDTCAACGSHF
EAGAGLMGRCEQGHVMDQDWNAAGNIYAGRAGATAQAAVG
(SEQ ID NO: 115)
>PJQE01000204_1M
[biofilm metagenome]
MKRKERKRPYYPVTVFSFHLEPHSIPQQVWDTAKEMQALW
NLMTEAHKRILEQTAEMEKPQKVEPYRAFYKEVYNLTKAA
PLNAGCKWDVHDRFIATLKRFHKKQGGPPKFKKGLDRVRI
VRRFFDGGRPIEWLFSRTSDRQPVRLRQVAENRQGRLMRG
FFNVGDEQMDFEAVQHRAIPQGSFLKQIALTGRLIRAFND
AEHRGWQWSLNLTVEIPPQAMAASVGRVAALDVGWRVRGD
YLRIGLLTDTDGNLYELRLPFDLSNRSARRCEGGGYQMVS
SFKQMWEIESEQDEYLESVKERVRPRFDEMPEELRPDKGS
WHKVREGGLRRLLWSLVEANACPDVREILTRWRERNGAFE
KQIAGGRERMLKRREWMYRNIAAWIADRYDELVWEGDFNL
STVAADDTDDPALKAAQRYRQMAALYTLRQAIGQAMRKRG
RVIVESPAAYSTQTCSICKAHVPSGADLILRCESGHLMDQ
DVNAARIMLGERVGELRAMAGVSTSVDRSQVSGVVVPLNV
DAKVRG
(SEQ ID NO: 116)
>3300006845|Ga007542l_100006223_8|P
[plants-rhizosphere-populus rhizosphere]
MIDEKTAKRKSPIRRGRLNKDWPIKVYTYDCWPQEKPPQA
LWDTAHEMRRLWNDFTAIFRKILADDKKDEAGKPLLSKEE
RAILWAPINTKPLREIAKQRKEKLDAGCREAVVVRFLTTV
GNWRKNPSRVGPPRFKRADDMNAISIPLVYNDGKSAEWLN
GGEGTSSIRDTRNLKTEAPEEYLNNGHFCVGITRERLNLH
VAYGGRSGRNKYYLPAECRIKQVALCGRRDSAFSWSWSFQ
VRLEHPPDPARRPTGRVCGWDSGGWRLMDGYIRLGVIADN
AGHFYELMVPLVIGAASRRMRREKEYCHKHGWEYSKPMTF
DDAEALDASYGATLEACKTEVCKIFEEEKEDWPADAGKIM
SRIVKMRDMGLRRLRRKLEQIESEAKRTIDEWDAEAAKMN
ETIRAFEIHAYKAKRDAYRQIAAWLNAFDIIAWEGDLSLK
QMAEQVGKKKQKRKEAHKETGQWNERTPEELQFEASQKYR
QIAGQHGLREFVKQKHKSRLQNEKAAYSTQTCPECGGAIE
PGHKLLLVCENGHGRDQDVTAALHFLNKIEGVASIMVPPV
EIPAHLRPYLRVMDASEARLELAEKL
(SEQ ID NO: 117)
>3300006847|Ga0075431_100017297_1|M
[plants-rhizosphere-populus rhizosphere]
MIDEKTAKRKSPIRRGRLNKDWPIKVYTYDCWPQEKPPQA
LWDTAHEMRRLWNDFTAIFRKILADDKKDEAGKPLLSKEE
RAILWAPINTKPLREIAKQRKEKLDAGCREAVVVRFLTTV
GNWRKNPSRVGPPRFKRADDMNAISIPLVYNDGKSAEWLN
GGEGTSSIRDTRNLKTEAPEEYLNNGHFCVGITRERLNLH
VAYGGRSGRNKYYLPAECRIKQVALCGRRDSAFSWSWSFQ
VRLEHPPDPARRPTGRVCGWDSGGWRLMDGYIRLGVIADN
AGHFYELMVPLVIGAASRRMRREKEYCHKHGWEYSKPMTF
DDAEALDASYGATLEACKTEVCKIFEEEKEDWPADAGKIM
SRIVKMRDMGLRRLRRKLEQIESEAKRTIDEWDAEAAKMN
ETIRAFEIHAYKAKRDAYRQIAAWLNAFDIIAWEGDLSLK
QMAEQVGKKKQKRKEAHKETGQWNERTPEELQFEASQKYR
QIAGQHGLREFVKQKHKSRLQNEKAAYSTQTCPECGGAIE
PGHKLLLVCENGHGRDQDVTAALHFLNKIEGVASIMVPPV
EIPAHLRPYLRVMDASEARLELAEKL
(SEQ ID NO: 118)
>3300027909|Ga0209382_10008589_8|P
[plants-rhizosphere-populus rhizosphere]
MIDEKTAKRKSPIRRGRLNKDWPIKVYTYDCWPQEKPPQA
LWDTAHEMRRLWNDFTAIFRKILADDKKDEAGKPLLSKEE
RAILWAPINTKPLREIAKQRKEKLDAGCREAVVVRFLTTV
GNWRKNPSRVGPPRFKRADDMNAISIPLVYNDGKSAEWLN
GGEGTSSIRDTRNLKTEAPEEYLNNGHFCVGITRERLNLH
VAYGGRSGRNKYYLPAECRIKQVALCGRRDSAFSWSWSFQ
VRLEHPPDPARRPTGRVCGWDSGGWRLMDGYIRLGVIADN
AGHFYELMVPLVIGAASRRMRREKEYCHKHGWEYSKPMTF
DDAEALDASYGATLEACKTEVCKIFEEEKEDWPADAGKIM
SRIVKMRDMGLRRLRRKLEQIESEAKRTIDEWDAEAAKMN
ETIRAFEIHAYKAKRDAYRQIAAWLNAFDIIAWEGDLSLK
QMAEQVGKKKQKRKEAHKETGQWNERTPEELQFEASQKYR
QIAGQHGLREFVKQKHKSRLQNEKAAYSTQTCPECGGAIE
PGHKLLLVCENGHGRDQDVTAALHFLNKIEGVASIMVPPV
EIPAHLRPYLRVMDASEARLELAEKL
(SEQ ID NO: 119)
>ODAK010035847_2|M
[soil metagenome]
MAYKFRAYVTGTDASHGQMLPPAIYTVAKNMENCWSDIAN
HNEQEYGAWKAAHVGADGKLPINIKSKTPIKPPKEFYAAS
EAWAIERIEQAQLPDELGTTILDRLHTTYKMMKRGGGPPK
PKRRMDRFALHHRYTGGGMPVANLGGQRAERFRVLLPSGT
AYQPWGRVKNPRDRRRERTVKAWFRVGDQPIQLRVVMHRP
LSVEDCFVKRVYLTGEKPSVAMPWVYYLVILIELPKPIVK
NTNAATVGIDVGWRKLDDERLRVAVGYDGHSHPELVLPLT
FQARGLGEVSLDRISKVKTLRDNSLEATKDKLRTLLNPLP
PGFAQMRNGGLLRLLRLMQQNEPDSPVIGMLENWKTDNDR
YLRLERVLENKLMAHRDKMYEKWALVEIATKYGVVKIEKM
NQQEMWDAEANKKDPALRAAAERRKYASCGALLAYIRRTV
KRVGGEIVEVPAEHTTDICSECGALFEAGSEIIGRCTRGH
EKDQDWNAAENIYAYSSAPVAELLSA
(SEQ ID NO: 120)
>SRR6266511_1572288_18|M
[soil metagenome]
MMQDTLQLKQRKNLARRERLNKDWPIKVYTYACWPQGTIP
SALWDTAHEMRRLWNDFTAIFRDILGKDLKDELGKPLLSK
QERAILWAPINTKPLREIAKQRKDRLDAGAGEGVVVRFMT
TVGNWRKNPARYGPPRFQRADEMDTILIPLVYNDGKSAEW
LLSGNGTSAVRDTRQLKTAAPQEYLNNGHFCIGITREKLN
LHIAYGGRSGQKKHHLPAKCRIKQVALAGKRDSVFGWSWS
FQVRLEYPPEPARQSTGRVCGWDSAGWRKMDDYIRLGVIA
DNAGHFYELRVPLAIGAASKRVRRERKHCQKNGWEYSKPI
TFDDAQALHSRYGTALEACKANVHKIFEEEKETWPEDARK
IMGGIVRMRDSGLRRLRQKLAPIDSVAKQTIDDWDVEAAS
LNGTIRAFEKHVDNVKKGAYRQIAAWLNTFDRVVWEGDLN
LKQLAEAAGGKKEKRKKQYAETGKYGERTSEDRQLEASQR
YRQIAGLYMLGQFVFEKIAICECSHSTQQHFRQIGPCHCG
CESYQEARLIDGSTAYSTQTCPECGASIEAGGKLLLVCEN
GHKRDQDCATSLYFLNKIEGRASISAPPLKIPAHLKPYLR
VMDASEVRLELVDKQ
(SEQ ID NO: 121)
>SRR6266511_203987_6|P
[soil metagenome]
MNTKSNMRVKGETQETPLASIPPRSTAATSGSAPAPPGPG
SSIVKDNRKKTPRRERLHKDWPIKVLVYPCWPIGEPPVEL
WDTAHEMRRLWNAFASLFRDALEADVKDEAGKPSLSKEDR
AKVWAPINMNNLREVGKERKEKLDQACRESVVNRFLKTVN
NWRKNSRENGPPRFRRAAEMDTVSIPLEYNYGKSAEWLNK
GEGTAGVRYTLDIKKNAPRDGNGAPLLSEEDLLAYLNNGH
FSVGVSRARLDLHIAYGGRSGKKKYFLPPGCRIKAVSLCG
KRDSAHGWSWSFQVRLEHPPGSSRPLTGRVCGWDAVGWRL
MDGYIRIGVIADNAGHFYEVRVPLAIGELSRRVRREKAHC
EKQGWEYTKPTTWDDLRELDSRYSKALDACKNEVRKTYEE
EKDKWPPEARRRMGGFAKMRDSGLRRLRRKLEESDSAAKE
TIDNWDAEAAQLNKMIRAFEIHAAMAKRDAFRQIAAWLDA
FDRVAWSAEPGLKSMAEAAGKRKQRRKEKYLETGQWEERT
PEERVFENSQKFRQIVGLHNLRQFVKERHGVGCFCGDHLL
QHEDNQGGCLACDCQAFNPRLMNCDAPYSTQACPECGGAI
KRGHKLLLRCENGHERDQDVSQSLHLLGSIEGYTSIAAPP
LDIPPHLRPYLRLMDASEAQITECARHSFRRRP
(SEQ ID NO: 122)
>SRR6266511_239355_1|M
[soil metagenome]
MEDISKKPERRKNFARRERLNKDWPFKVYPYRCMLREKPP
QALWDTAREMRRLWNDFTETFHKILKEDKKDEDGKPLLSK
EQRATLWASINIKPLREIAKKRKDKLDIGCREFIVTNFIT
TVGNWRKNPGKFGPPRFQPASEIRSIYIPLVYYDGKSAEW
LHCGKGTAWVQDTLHIKKNASRDEKGRPILTEEEMEQYLN
NSHFCVGITREKLNLHVAYGGRSGKKKRYLPDKCRIKWVA
LAGRRDSAFGWSWSFQVRLEHPPDPERQVTGRVCGWDSGG
WRKMDGYIRLGVIADNAGHFYELMVPLAIGAASHRLRHER
EHCQKNGWEYNKPITFDDAEALQSRYGLALEACKTNLRKI
FEEEKEKWPDDARKIMGGIVRMRDDGLRRLRRKLDAIESA
AKQTIDDWNAEAAKLNETIRAFQRHADNAKKDAYRQITAW
LSAFDRIAWEGVLNLKWMAEQPGKKKQERKKTLAETGQGG
ERTAEDRQLEASQRYRHIVGQYRLREFVKQTHEARLQNEK
TAYSTRTCPECGAHINPGGDLQLVCDNGHKRDQDVTTSLY
LLSKIEGYASASGPPIEIPAHLSQYLRVMHASEVRLEIVE
KH
(SEQ ID NO: 123)
>SRR6266511_838544_1|M
[soil metagenome]
MEDISKKPERRKNPARRERLNKDWPIKVHNYRCSLNEKPP
QTLWDTAHEMRRLWNDLTAVFRKILAEDKKDEDGKPLLSK
EERANLWAPINIKPLREIAKQRKDKLDIGCREYVVTHFIT
TVNNWRRNPRRFGPPRFQNAADMDSVHIPLVYTDGKSADW
LTGGNGTAWVRDTLHIKKNALRDKDGRPILSENELEEYLN
NGHFCVGITREKLSLHIAYGGRSGRKRYHLPDKCRIKQVA
LCGRRNSAFGWSWSFQVRLEHPPDPERQVTGRVCGWDSVG
WRKMDGYIRLGVIADNAGHFYELMVPLAIGAASHRLRHER
EHCQKNGWEYNKPITFDDAEALQSRYGLALEACKTNLRKI
FEEEKEKWPDDARKIMGGIVRMRDDGLRRLRRNIDAIESA
AKQTIDDWDAEAAKLNETIRAFQRHADNAKKDAYRQITAW
LSAFDKIAWEGDRSLKGMSEQGGKEKQKRKKMHEETGQWG
ERTAEERQLEASQRNRQIVGQHRLREFVKRMHADRLQNEK
TAHSTRICPECGAHINPGGDLQLVCDNGHKRDQDVTTSLY
LLSKIEGCASASGPPIEIPAHLSPYLRVMTASEVALEIVE
(SEQ ID NO: 124)
>3300014502|Ga0182021_10233972_2|M
[terrestrial-soil-fen]
MRPKKNADTIYQTYKFWIKPSDPLPQLLWDKAKAMNAAWT
SLVLMRQETLETWMAQRDTLTADQKRAFWNTFNLAVREKI
EECELDWETSGVLHDRFLVAHQRALKDKADLHGRTRLQRI
LLVHRYTGGGIPIEKLLSTRAWRFALDAFPTEDVYATNTR
QARRGRQTQGRFGLDNETAVEFSIILHRPLPPAAIVKRVA
LSGRRQKPPAHEWDWALVVSVEVPNPSRTAAGRVIGIDLG
WRKIDDYLRIGYAADDTDQQIELRLPLDFSTQKARSAGYP
CNHAELRALEGRIAVAVDATK
AAIGELIAVPSLALMRQGGLVRLQRTLSEKPDLSEAEVEA
LRILGAWRKQNDRLQKIYNFSTARFAARRRWLYQNLAAWL
CRNYDVIAIEGDLDVKGLAEEPDPDPALKAAQRYRQIAAP
GELREIIRWTAQREGVIVKDCNTAYSTTTCFTCGEQAEAG
PDLILECPAGHRFDQDANAAANLLSQILPSTEVQDGLRKN
QRDFLRIPPELQMVAWVHRD
(SEQ ID NO: 125)
>3300005529|Ga0070741_10001368_72|P
[terrestrial-soil-surface soil]
MSAIIEVQKPQHRKERANPDWPVKVYSYWIEPIGDIPEWF
WLFHDHMQLLWNDMVIAHDAFIDEREFSEESDGKWDTDRK
RLEFSGLREKLKNVAKSKKGILPSSCYYEVRDRFLITMGR
YWNPDSTLKRRRNETKNFDLGRPGMKFGRNKVLIPHDLNA
ANISTSILFSNKKPLTILQRTKQQGPTRGIFETLGYNFNF
RINLHRPIPNGNIKRIAIVGEMVKPFGWQWRLNLTIEEPP
VRPQRKNGRVAAIDLGWRLMDDCIRIGVLVDNSDNAIELS
IPLDFANNSLRRQREHFMDVTDACLPIHSIPDIWKAQQES
DLILEVCKNRLTALIPELLPNKVAWDKARHKSLIKLLNTL
REDHPESSAIAVIEQWRTTNWVLHRRWRAAYERFMRWRRE
IYRVLAAEIASTYDEIVLEDRLNLAEMSRDEQAPPAIKAS
QKYRHWTSLYLFKECLKYAMDKQQKLIIAGKTAYSTATCS
TCDGKINTDASLYLTCANGHTLDQDLNAARNLLKSYPECE
SLPGLESPIRHSQLSKCVRTLSLLKSL
(SEQ ID NO: 126)
>3300010046|Ga0126384_10047357_3|M
[terrestrial-soil-tropical forest soil]
MMQDTLLLKQRKNLARRERLNKDWPIKVYTYACWPQGTIP
SALWDTAHEMRRLWNDFTAIFRDILSKDLKDELGKPLLSK
QERAILWAPINTKPLREIAKQRKDELDAGTREGVVVRFMT
TVGNWRKNPARYGPPRFQRADEMDTILIPLVYNDGKSAEW
LLSGNGTSAVRDTRQLKTAAPQEYLNNGHFCIGITREKLN
LHIAYGGRSGQKKHHLPAKCRIKQVALAGKRDSVFGWSWS
FQVRLEYPPEPARQSTGRVCGWDSAGWRKMDDYIRLGVIA
DNAGHFYELRVPLAIGAASKRVRRERKHCQKNGWEYSKPI
TFDDAQALHSRYGTALEACKANVHKIFEEEKETWPEDARK
IMGGIVRMRDSGLRRLRQKLAPIDSVAKQTIDDWDVEAAS
LNETIRAFEKHVDNVKKGAYRQIAAWLNTFDRVVWEGDLN
LKQLAEAAGGKKEKRKKQYAETGKYGERTPEDRQLEASQR
YRQIAGLYMLRQFVFEKIAICECSHSTQQHFRQIGPCHCG
CESYQEARLIDGSTAYSTQTCPECGASIEAGGKLLLVCEN
GHKRDQDCATSLYFLNKMEGRASISAPPLKIPAHLKPYLR
VMDASEVRLELVDKQ
(SEQ ID NO: 127)
>3300010359|Ga0126376_10030051_2|M
[terrestrial-soil-tropical forest soil]
MMQDTLLLKQRKNLARRERLNKDWPIKVYTYACWPQGTIP
SALWDTAHEMRRLWNDFTAIFRDILSKDLKDELGKPLLSK
QERAILWAPINTKPLREIAKQRKDELDAGTREGVVVRFMT
TVGNWRKNPARYGPPRFQRADEMDTILIPLVYNDGKSAEW
LLSGNGTSAVRDTRQLKTAAPQEYLNNGHFCIGITREKLN
LHIAYGGRSGQKKHHLPAKCRIKQVALAGKRDSVFGWSWS
FQVRLEYPPEPARQSTGRVCGWDSAGWRKMDDYIRLGVIA
DNAGHFYELRVPLAIGAASKRVRRERKHCQKNGWEYSKPI
TFDDAQALHSRYGTALEACKANVHKIFEEEKETWPEDARK
IMGGIVRMRDSGLRRLRQKLAPIDSVAKQTIDDWDVEAAS
LNETIRAFEKHVDNVKKGAYRQIAAWLNTFDRVVWEGDLN
LKQLAEAAGGKKEKRKKQYAETGKYGERTPEDRQLEASQR
YRQIAGLYMLRQFVFEKIAICECSHSTQQHFRQIGPCHCG
CESYQEARLIDGSTAYSTQTCPECGASIEAGGKLLLVCEN
GHKRDQDCATSLYFLNKIEGRASISAPPHKIPAHLKPYLR
VMDASEVRLELVDKQ
(SEQ ID NO: 128)
>3300010362|Ga0126377_10111069_2|M
[terrestrial-soil-tropical forest soil]
MMQDTLLLKQRKNLARRERLNKDWPIKVYTYACWPQGTIP
SALWDTAHEMRRLWNDFTAIFRDILSKDLKDELGKPLLSK
RERAILWAPINTKPLREIAKQRKDELDAGAREGVVVRFMT
TVGNWRKNPARYGPPRFQRADEMDTILIPLVYNDGKSAEW
LLSGNGTSAVHDTRQLKTAAPQEYLNNGHFCIGITREKLN
LHIAYGGRSGQKKHHLPAKCRIKQVALAGKRDSVFGWSWS
FQVRLEYPPEPARQSTGRVCGWDSAGWRKMDDYIRLGVIA
DNAGHFYELRVPLAIGAASKRVRRERKHCQKNGWEYSKPI
TFDDAQALHSRYGTALEACKANVHKIFEEEKETWPEDARK
IMGGIVRMRDSGLRRLRQKLAPIDSVAKQTIDDWDVEAAS
LNETIRAFEKHVDNVKKGAYRQIAAWLNTFDRVVWEGDLN
LKQLAEAAGRKKEKRKKQYAETGKYGERTPEDRQLEASQR
YRQIAGLYMLRQFVFEKIAICECSHSTQQHFRQIGPCHCG
CESYQEARLIDGSTAYSTQTCPECGASIEAGGKLLLVCEN
GHKRDQDCATSLYFLNKIEGRASISAPPLKIPAHLKPYLR
VMDASEVRLELVDKQ
(SEQ ID NO: 129)
>3300012204|Ga0137374_10002290_4|M
[terrestrial-soil-vadose zone soil]
MNTKSNMRVKGETQETPLASIPPRSTAATSGSAPAPPGPG
SSIVKDNRKKTPRRERLHKDWPIKVLVYPCWPIGEPPVEL
WDTAHEMRRLWNAFASLFRDALEADVKDEAGKPSLSKEDR
AKVWAPINMNNLREVGKERKEKLDQACRESVVNRFLKTVN
NWRKNSRENGPPRFRRAAEMDTVSIPLEYNYGKSAEWLNK
GEGTAGVRYTLDIKKNAPRDGNGAPLLSEEDLLAYLNNGH
FSVGVSRARLDLHIAYGGRSGKKKYFLPPGCRIKAVSLCG
KRDSAHGWSWSFQVRLEHPPGSSRPLTGRVCGWDAVGWRL
MDGYIRIGVIADNAGHFYEVRVPLAIGELSRRVRREKAHC
EKQGWEYTKPTTWDDLRELDSRYSKALDACKNEVRKTYEE
EKDKWPPEARRRMGGFAKMRDSGLRRLRRKLEESDSAAKE
TIDNWDAEAAQLNKMIRAFEIHAAMAKRDAFRQIAAWLDA
FDRVAWSAEPGLKSMAEAAGKRKQRRKEKYLETGQWEERT
PEERVFENSQKFRQIVGLHNLRQFVKERHGVGCFCGDHLL
QHEDNQGGCLACDCQAFNPRLMNCDAPYSTQACPECGGAI
KRGHKLLLRCENGHERDQDVSQSLHLLGSIEGYTSIAAPP
LDIPPHLRPYLRLMDASEAQITECARHSFRRRP
(SEQ ID NO: 130)
>3300012204|Ga0137374_100098796|P
[terrestrial-soil-vadose zone soil]
MMQDTLQLKQRKNLARRERLNKDWPIKVYTYACWPQGTIP
SALWDTAHEMRRLWNDFTAIFRDILGKDLKDELGKPLLSK
QERAILWAPINTKPLREIAKQRKDRLDAGAGEGVVVRFMT
TVGNWRKNPARYGPPRFQRADEMDTILIPLVYNDGKSAEW
LLSGNGTSAVRDTRQLKTAAPQEYLNNGHFCIGITREKLN
LHIAYGGRSGQKKHHLPAKCRIKQVALAGKRDSVFGWSWS
FQVRLEYPPEPARQSTGRVCGWDSAGWRKMDDYIRLGVIA
DNAGHFYELRVPLAIGAASKRVRRERKHCQKNGWEYSKPI
TFDDAQALHSRYGTALEACKANVHKIFEEEKETWPEDARK
IMGGIVRMRDSGLRRLRQKLAPIDSVAKQTIDDWDVEAAS
LNGTIRAFEKHVDNVKKGAYRQIAAWLNTFDRVVWEGDLN
LKQLAEAAGGKKEKRKKQYAETGKYGERTSEDRQLEASQR
YRQIAGLYMLGQFVFEKIAICECSHSTQQHFRQIGPCHCG
CESYQEARLIDGSTAYSTQTCPECGASIEAGGKLLLVCEN
GHKRDQDCATSLYFLNKIEGRASISAPPLKIPAHLKPYLR
VMDASEVRLELVDKQ
(SEQ ID NO: 131)
>3300012350|Ga0137372_10022207_3|M
[terrestrial-soil-vadose zone soil]
MMQDTLQLKQRKNLARRERLNKDWPIKVYTYACWPQGTIP
SALWDTAHEMRRLWNDFTAIFRDILGKDLKDELGKPLLSK
QERAILWAPINTKPLREIAKQRKDRLDAGAGEGVVVRFMT
TVGNWRKNPARYGPPRFQRADEMDTILIPLVYNDGKSAEW
LLSGNGTSAVRDTRQLKTAAPQEYLNNGHFCIGITREKLN
LHIAYGGRSGQKKHHLPAKCRIKQVALAGKRDSVFGWSWS
FQVRLEYPPEPARQSTGRVCGWDSAGWRKMDDYIRLGVIA
DNAGHFYELRVPLAIGAASKRVRRERKHCQKNGWEYSKPI
TFDDAQALHSRYGTALEACKANVHKIFEEEKETWPEDARK
IMGGIVRMRDSGLRRLRQKLAPIDSVAKQTIDDWDVEAAS
LNGTIRAFEKHVDNVKKGAYRQIAAWLNTFDRVVWEGDLN
LKQLAEAAGGKKEKRKKQYAETGKYGERTSEDRQLEASQR
YRQIAGLYMLGQFVFEKIAICECSHSTQQHFRQIGPCHCG
CESYQEARLIDGSTAYSTQTCPECGASIEAGGKLLLVCEN
GHKRDQDCATSLYFLNKIEGRASISAPPLKIPAHLKPYLR
VMDASEVRLELVDKQ
(SEQ ID NO: 132)
>3300012350|Ga0137372_10022207_4|P
[terrestrial-soil-vadose zone soil]
MQDTLQLKQRKNLARRERLNKDWPIKVYTYACWPQGTIPS
ALWDTAHEMRRLWNDFTAIFRDILGKDLKDELGKPLLSKQ
ERAILWAPINTKPLREIAKQRKDRLDAGAGEGVVVRFMTT
VGNWRKNPARYGPPRFQRADEMDTILIPLVYNDGKSAEWL
LSGNGTSAVRDTRQLKTAAPQEYLNNGHFCIGITREKLNL
HIAYGGRSGQKKHHLPAKCRIKQVALAGKRDSVFGWSWSF
QVRLEYPPEPARQSTGRVCGWDSAGWRKMDDYIRLGVIAD
NAGHFYELRVPLAIGAASKRVRRERKHCQKNGWEYSKPIT
FDDAQALHSRYGTALEACKANVHKIFEEEKETWPEDARKI
MGGIVRMRDSGLRRLRQKLAPIDSVAKQTIDDWDVEAASL
NGTIRAFEKHVDNVKKGAYRQIAAWLNTFDRVVWEGDLNL
KQLAEAAGGKKEKRKKQYAETGKYGERTSEDRQLEASQRY
RQIAGLYMLGQFVFEKIAICECSHSTQQHFRQIGPCHCGC
ESYQEARLIDGSTAYSTQTCPECGASIEAGGKLLLVCENG
HKRDQDCATSLYFLNKIEGRASISAPPLKIPAHLKPYLRV
MDASEVRLELVDKQ
(SEQ ID NO: 133)
>3300012353|Ga0137367_10000906_4|M
[terrestrial-soil-vadose zone soil]
MNTKSNMRVKGETQETPLASIPPRSTAATSGSAPAPPGPG
SSIVKDNRKKTPRRERLHKDWPIKVLVYPCWPIGEPPVEL
WDTAHEMRRLWNAFASLFRDALEADVKDEAGKPSLSKEDR
AKVWAPINMNNLREVGKERKEKLDQACRESVVNRFLKTVN
NWRKNSRENGPPRFRRAAEMDTVSIPLEYNYGKSAEWLNK
GEGTAGVRYTLDIKKNAPRDGNGAPLLSEEDLLAYLNNGH
FSVGVSRARLDLHIAYGGRSGKKKYFLPPGCRIKAVSLCG
KRDSAHGWSWSFQVRLEHPPGSSRPLTGRVCGWDAVGWRL
MDGYIRIGVIADNAGHFYEVRVPLAIGELSRRVRREKAHC
EKQGWEYTKPTTWDDLRELDSRYSKALDACKNEVRKTYEE
EKDKWPPEARRRMGGFAKMRDSGLRRLRRKLEESDSAAKE
TIDNWDAEAAQLNKMIRAFEIHAAMAKRDAFRQIAAWLDA
FDRVAWSAEPGLKSMAEAAGKRKQRRKEKYLETGQWEERT
PEERVFENSQKFRQIVGLHNLRQFVKERHGVGCFCGDHLL
QHEDNQGGCLACDCQAFNPRLMNCDAPYSTQACPECGGAI
KRGHKLLLRCENGHERDQDVSQSLHLLGSIEGYTSIAAPP
LDIPPHLRPYLRLMDASEAQITECARHSFRRRP
(SEQ ID NO: 134)
>3300012353|Ga0137367_10004593_5|M
[terrestrial-soil-vadose zone soil]
MMQDTLQLKQRKNLARRERLNKDWPIKVYTYACWPQGTIP
SALWDTAHEMRRLWNDFTAIFRDILGKDLKDELGKPLLSK
QERAILWAPINTKPLREIAKQRKDRLDAGAGEGVVVRFMT
TVGNWRKNPARYGPPRFQRADEMDTILIPLVYNDGKSAEW
LLSGNGTSAVRDTRQLKTAAPQEYLNNGHFCIGITREKLN
LHIAYGGRSGQKKHHLPAKCRIKQVALAGKRDSVFGWSWS
FQVRLEYPPEPARQSTGRVCGWDSAGWRKMDDYIRLGVIA
DNAGHFYELRVPLAIGAASKRVRRERKHCQKNGWEYSKPI
TFDDAQALHSRYGTALEACKANVHKIFEEEKETWPEDARK
IMGGIVRMRDSGLRRLRQKLAPIDSVAKQTIDDWDVEAAS
LNGTIRAFEKHVDNVKKGAYRQIAAWLNTFDRVVWEGDLN
LKQLAEAAGGKKEKRKKQYAETGKYGERTSEDRQLEASQR
YRQIAGLYMLGQFVFEKIAICECSHSTQQHFRQIGPCHCG
CESYQEARLIDGSTAYSTQTCPECGASIEAGGKLLLVCEN
GHKRDQDCATSLYFLNKIEGRASISAPPLKIPAHLKPYLR
VMDASEVRLELVDKQ
(SEQ ID NO: 135)
>3300012355|Ga0137369_10001944_2|P
[terrestrial-soil-vadose zone soil]
MNTKSNMRVKGETQETPLASIPPRSTAATSGSAPAPPGPG
SSIVKDNRKKTPRRERLHKDWPIKVLVYPCWPIGEPPVEL
WDTAHEMRRLWNAFASLFRDALEADVKDEAGKPSLSKEDR
AKVWAPINMNNLREVGKGWKEKLDQACRESVVNRFLKTVN
NWRKNSRENGPPRFRRAAEMDTVSIPLEYNYGKSAEWLNK
GEGTAGVRYTLDIKKNAPRDGNGAPLLSEEDLLAYLNNGH
FSVGVSRARLDLHIAYGGRSGKKKYFLPPGCRIKAVSLCG
KRDSAHGWSWSFQVRLEHPPGSSRPLTGRVCGWDAVGWRL
MDGYIRIGVIADNAGHFYEVRVPLAIGELSRRVRREKAHC
EKQGWEYTKPTTWDDLRELDSRYSKALDACKNEVRKTYEE
EKDKWPPEARRRMGGFAKMRDSGLRRLRRKLEESDSAAKE
TIDNWDAEAAQLNKMIRAFEIHAAMAKRAAFRQIAAWLDA
FDRVAWSAEPGLKSMAEAAGKRKQRRKEKYLETGQWEERT
PEERVFENSQKFRQIVGLHNLRQFVKERHGVGCFCGDHLL
QHEDNQGGCLACDCQAFNPRLMNCDAPYSTQACPECGGAI
KRGHKLLLRCENGHERDQDVSQSLHLLGSIEGYTSIAAPP
LDIPPHLRPYLRLMDASEAQITECARHSFRRRP
(SEQ ID NO: 136)
>3300012355|Ga0137369_10007378_6|M
[terrestrial-soil-vadose zone soil]
MMQDTLQLKQRKNLARRERLNKDWPIKVYTYACWPQGTIP
SALWDTAHEMRRLWNDFTAIFRDILGKDLKDELGKPLLSK
QERAILWAPINTKPLREIAKQRKDRLDAGAGEGVVVRFMT
TVGNWRKNPARYGPPRFQRADEMDTILIPLVYNDGKSAEW
LLSGNGTSAVRDTRQLKTAAPQEYLNNGHFCIGITREKLN
LHIAYGGRSGQKKHHLPAKCRIKQVALAGKRDSVFGWSWS
FQVRLEYPPEPARQSTGRVCGWDSAGWRKMDDYIRLGVIA
DNAGHFYELRVPLAIGAASKRVRRERKHCQKNGWEYSKPI
TFDDAQALHSRYGTALEACKANVHKIFEEEKETWPEDARK
IMGGIVRMRDSGLRRLRQKLAPIDSVAKQTIDDWDVEAAS
LNGTIRAFEKHVDNVKKGAYRQIAAWLNTFDRVVWEGDLN
LKQLAEAAGGKKEKRKKQYAETGKYGERTSEDRQLEASQR
YRQIAGLYMLGQFVFEKIAICECSHSTQQHFRQIGPCHCG
CESYQEARLIDGSTAYSTQTCPECGASIEAGGKLLLVCEN
GHKRDQDCATSLYFLNKIEGRASISAPPLKIPAHLKPYLR
VMDASEVRLELVDKQ
(SEQ ID NO: 137)
>3300012355|Ga0137369_10043960_1|M
[terrestrial-soil-vadose zone soil]
MEDISKKPERRKNFARRERLNKDWPFKVYPYRCMLREKPP
QALWDTAREMRRLWNDFTETFHKILKEDKKDEEGRPLLSK
EQRATLWASINIKPLREIAKKRKDKLDIGCREFIVTNFIT
TVGNWRKNPGKFGPPRFQPASEIRSIYIPLVYYDGKSAEW
LHCGKGTAWVQDTLHIKKNASRDEKGRPILTEEEMEQYLN
NSHFCVGITREKLNLHVAYGGRSGKKKRYLPDKCRIKWVA
LAGRRDSAFGWSWSFQVRLEHPPDPERQVTGRVCGWDSGG
WRKMDGYIRLGVIADNAGHFYELMVPLAIGAASHRLRHER
EHCQKNGWEYNKPITFDDAEALQSRYGLALEACKTNLRKI
FEEEKEKWPDDARKIMGGIVRMRDDGLRRLRRKLDAIESA
AKQTIDDWNAEAAKLNETIRAFQRHADNAKKDAYRQITAW
LSAFDRIAWEGDLNLKWMAEQPGKKKQERKKTLAETGQGG
ERTAEDRQLEASQRYRHIVGQYRLREFVKQTHEARLQNEK
TAHSTRTCPECGAHINPGGDLQLVCDNGHKRDQDVTTSLY
LLSKIEGCARVSAPPIEIPAHLSQYLRVMHASEVRLEIVE
KH
(SEQ ID NO: 138)
>3300012358|Ga0137368_10001884_4|M
[terrestrial-soil-vadose zone soil]
MNTKSNMRVKGETQETPLASIPPRSTAATSGSAPAPPGPG
SSIVKDNRKKTPRRERLHKDWPIKVLVYPCWPIGEPPVEL
WDTAHEMRRLWNAFASLFRDALEADVKDEAGKPSLSKEDR
AKVWAPINMNNLREVGKERKEKLDQACRESVVNRFLKTVN
NWRKNSRENGPPRFRRAAEMDTVSIPLEYNYGKSAEWLNK
GEGTAGVRYTLDIKKNAPRDGNGAPLLSEEDLLAYLNNGH
FSVGVSRARLDLHIAYGGRSGKKKYFLPPGCRIKAVSLCG
KRDSAHGWSWSFQVRLEHPPGSSRPLTGRVCGWDAVGWRL
MDGYIRIGVIADNAGHFYEVRVPLAIGELSRRVRREKAHC
EKQGWEYTKPTTWDDLRELDSRYSKALDACKNEVRKTYEE
EKDKWPPEARRRMGGFAKMRDSGLRRLRRKLEESDSAAKE
TIDNWDAEAAQLNKMIRAFEIHAAMAKRDAFRQIAAWLDA
FDRVAWSAEPGLKSMAEAAGKRKQRRKEKYLETGQWEERT
PEERVFENSQKFRQIVGLHNLRQFVKERHGVGCFCGDHLL
QHEDNQGGCLACDCQAFNPRLMNCDAPYSTQACPECGGAI
KRGHKLLLRCENGHERDQDVSQSLHLLGSIEGYTSIAAPP
LDIPPHLRPYLRLMDASEAQITECARHSFRRRP
(SEQ ID NO: 139)
>3300012358|Ga0137368_10007571_6|P
[terrestrial-soil-vadose zone soil]
MMQDTLQLKQRKNLARRERLNKDWPIKVYTYACWPQGTIP
SALWDTAHEMRRLWNDFTAIFRDILGKDLKDELGKPLLSK
QERAILWAPINTKPLREIAKQRKDRLDAGAGEGVVVRFMT
TVGNWRKNPARYGPPRFQRADEMDTILIPLVYNDGKSAEW
LLSGNGTSAVRDTRQLKTAAPQEYLNNGHFCIGITREKLN
LHIAYGGRSGQKKHHLPAKCRIKQVALAGKRDSVFGWSWS
FQVRLEYPPEPARQSTGRVCGWDSAGWRKMDDYIRLGVIA
DNAGHFYELRVPLAIGAASKRVRRERKHCQKNGWEYSKPI
TFDDAQALHSRYGTALEACKANVHKIFEEEKETWPEDARK
IMGGIVRMRDSGLRRLRQKLAPIDSVAKQTIDDWDVEAAS
LNGTIRAFEKHVDNVKKGAYRQIAAWLNTFDRVVWEGDLN
LKQLAEAAGGKKEKRKKQYAETGKYGERTSEDRQLEASQR
YRQIAGLYMLGQFVFEKIAICECSHSTQQHFRQIGPCHCG
CESYQEARLIDGSTAYSTQTCPECGASIEAGGKLLLVCEN
GHKRDQDCATSLYFLNKIEGRASISAPPLKIPAHLKPYLR
VMDASEVRLELVDKQ
(SEQ ID NO: 140)
>3300012360|Ga0137375_10064112_1|P
[terrestrial-soil-vadose zone soil]
MNTKSNMRVKGETQETPLASIPPRSTAATSGSAPAPPGPG
SSIVKDNRKKTPRRERLHKDWPIKVLVYPCWPIGEPPVEL
WDTAHEMRRLWNAFVSLFRDALEADVKDEAGKPSLSKEDR
AKVWAPINMNNLREVGKERKEKLDQACRESVVNRFLKTVN
NWRKNSRENGPPRFRRAAEMDTVSIPLEYNYGKSAEWLNK
GEGTAGVRYTLDIKKNAPRDGNGAPLLSEEDLLAYLNNGH
FSVGVSRARLDLHIAYGGRSGKKKYFLPPGCRIKAVSLCG
KRDSAHGWSWSFQVRLEHPPGSSRPLTGRVCGWDAVGWRL
MDGYIRIGVIADNAGHFYEVRVPLAIGELSRRVRREKAHC
EKQGWEYTKPTTWDDLRELDSRYSKALDACKNEVRKTYEE
EKDKWPPEARRRMGGFAKMRDSGLRRLRRKLEESDSAAKE
TIDNWDAEAAQLNKMIRAFEIHAAMAKRDAFRQIAAWLDA
FDRVAWSAEPGLKSMAEAAGKRKQRRKEKYLETGQWEERT
PEERVFENSQKFRQIVGLHNLRQFVKERHGVGCFCGDHLL
QHEDNQGGCLACDCQAFNPRLMNCDAPYSTQACPECGGAI
KRGHKLLLRCENGHERDQDVSQSLHLLGSIEGYTSIAAPP
LDIPPHLRPYLRLMDASEAQITECARHSFRRRP
(SEQ ID NO: 141)
>3300012532|Ga0137373_10033017_1|P
[terrestrial-soil-vadose zone soil]
(SEQ ID NO: 142)
MNTKSNMRVKGETQETPLASIPPRSTAATSGSAPAPPGPG
SSIVKDNRKKTPRRERLHKDWPIKVLVYPCWPIGEPPVEL
WDTAHEMRRLWNAFASLFRDALEADVKDEAGKPSLSKEDR
AKVWAPINMNNLREVGKERKEKLDQACRESVVNRFLKTVN
NWRKNSRENGPPRFRRAAEMDTVSIPLEYNYGKSAEWLNK
GEGTAGVRYTLDIKKNAPRDGNGAPLLSEEDLLAYLNNGH
FSVGVSRARLDLHIAYGGRSGKKKYFLPPGCRIKAVSLCG
KRDSAHGWSWSFQVRLEHPPGSSRPLTGRVCGWDAVGWRL
MDGYIRIGVIADNAGHFYEVRVPLAIGELSRRVRREKAHC
EKQGWEYTKPTTWDDLRELDSRYSKALDACKNEVRKTYEE
EKDKWPPEARRRMGGFAKMRDSGLRRLRRKLEESDSAAKE
TIDNWDAEAAQLNKMIRAFEIHAAMAKRDAFRQIAAWLDA
FDRVAWSAEPGLKSMAEAAGKRKQRRKEKYLETGOWEERT
PEERVFENSQKFRQIVGLYNLRQFVKERHGVGCFCGDHLL
QHEDNQGGCLACDCQAFNPRLMNCDAPYSTQACPECGGAI
KRGHKLLLRCENGHERDQDVSQSLHLLGSIEGYTSIAAPP
LDIPPHLRPYLRLMDASEAQITECARHSFRRRP
>3300012944|Ga0137410_10000075_50|P
[terrestrial-soil-vadose zone soil]
MTRKLNNPDMPTLAYKYDVRIIGDLPQEIWDTGKAMNALY
SDLAERHEAFCGAFKDASKEMRKEQFAKLTKNGKGGILYE
RAKASKDALNFRYYYTVHEQFRKSQQRFAKRQGGAPKPKR
CLERMLFPMEFGSFSEPTTWIHSNSENRAYVREAQGPTRG
HFVIHTPYDEITIPFEIVMDRPLPDGALIKGIALSGFHER
PFEWKWSLVFSLQVPPHPKAPSVGRVAGLDLGWRDMGDCI
RIGMLADSAGDFRELRLPYDLSRNQDRKFIKRLESQGVVD
RPMLRDIRLIRDAQRKMDDHLEACKIDLAGVDRSVWPDEA
RASMAGWKMRAGGLQRIRRTLFEAGISYEFLEDWHFWHDT
NLRRYRAAQIDWIAARDYIYRCIAAWIADNYDTVAWEGDL
GLKRMAEAAGKRKKARKEEHEETGEWQERTVDDRISEASQ
KRRQWASLHTLRGYIAEAMKKRGRDLQGHPAAYSTQTCDE
CGQHIAPGHELIRECAERHVEDQDATAALYYLRIVQDYLR
AVTGFSASVNHSQLLKAIKPISPMNAA
>3300015245|Ga0137409_10000164_50|P
[terrestrial-soil-vadose zone soil]
(SEQ ID NO: 144)
MTRKLNNPDMPTLAYKYDVRIIGDLPQEIWDTGKAMNALY
SDLAERHEAFCGAFKDASKEMRKEQFAKLTKNGKGGILYE
RAKASKDALNFRYYYTVHEQFRKSQQRFAKRQGGAPKPKR
CLERMLFPMEFGSFSEPTTWIHSNSENRAYVREAQGPTRG
HFVIHTPYDEITIPFEIVMDRPLPDGALIKGIALSGFHER
PFEWKWSLVFSLQVPPHPKAPSVGRVAGLDLGWRDMGDCI
RIGMLADSAGDFRELRLPYDLSRNQDRKFIKRLESQGVVD
RPMLRDIRLIRDAQRKMDDHLEACKIDLAGVDRSVWPDEA
RASMAGWKMRAGGLQRIRRTLFEAGISYEFLEDWHFWHDT
NLRRYRAAQIDWIAARDYIYRCIAAWIADNYDTVAWEGDL
GLKRMAEAAGKRKKARKEEHEETGEWQERTVDDRISEASQ
KRRQWASLHTLRGYIAEAMKKRGRDLQGHPAAYSTQTCDE
CGQHIAPGHELIRECAERHVEDQDATAALYYLRIVQDYLR
AVTGFSASVNHSQLLKAIKPISPMNAA
(SEQ ID NO: 143)
>SRR6837571_912213_1|M
[wastewater metagenome]
MRTTKTPDYSNKNLSFWCEPVGEMPSVVFEKGRQMNRLWN
LLVECHTTFLAKHDGKKGGERTEAYKTEWQPLLKRIYNNE
AKSFGLNSDETNFVTDGFKNAIRAGYDQKRPGAGMPTFKA
RLRSIHIPFCDRNGGRAPAWFFEKPDRRIHLRPILLPDAA
KRVIDRGPARGHFTVDGQAISLRANLSQSIPPGALVKRVS
LVGKLERPFGWQWKLMFSIAYPPPPPAQSDAACVGVDLGY
RRFEDRIRFGMIFDGSRFHELALPLDLTNRRAAKAGKRWD
IREIWELESQIGCGVEACKASLKQLPKTDWPEEARGAMQG
IAKMRERGLLKVRRLLRDAEITVPELETWWDDYQRLFRKS
RHLELQIVATRTHLYRNLALSLARSCAAIAWEGGLSLRDL
AESAGKKKKQRKEDIAEGVSAERSPEDRIEEAAQKWRAYV
CLSDFRRFLKEAVEKTTAALIDRPAAGTTNTCEECGAPVD
TGPDLVLTCANGHRRDQDKGSAKMLFEEIDEGYRQQSAPL
SLDLFTDQAVRAIRTLNREMV
(SEQ ID NO: 145)

TABLE 11
Nucleotide sequences of Representative CLUST.099129
Direct Repeats and Spacer Lengths

| | | Spacer |
|---|---|---|
| Effector Accession | Direct Repeat Nucleotide Sequence | Length(s) |
| SRR6837557_1470862_2\|M (SEQ ID | AGTCGCGAATAACTGTTCAGCGGAGAAGCCGCTG | 32-34 |
| NO: 101) | AAAC (SEQ ID NO: 146) | |
| | GTTTCAGCGGCTTCTCCGCTGAACAGTTATTCGC | |
| | GACT (SEQ ID NO: 200) | |
| 3300012971\|Ga0126369_10127246_ | GTGATTGCAGACTGGTCGTGCGAATGCGCACGGC | 34-36 |
| UM (SEQ ID NO: 102) | AC (SEQ ID NO: 147) | |
| | GTGCCGTGCGCATTCGCACGACCAGTCTGCAATC | |
| | AC (SEQ ID NO: 201) | |
| 3300005764\|Ga0066903_100343966 | AGTAATTGAAAGTTGGTCGTGCGAATCCGCACGG | 32-34 |
| _1\|M (SEQ ID NO: 103) | CAC (SEQ ID NO: 148) | |
| | GTGCCGTGCGGATTCGCACGACCAACTTTCAATT | |
| | ACT (SEQ ID NO: 202) | |
| PMDR01000701_10\|P (SEQ ID NO: | GTTGCAGTGGTTGTCCATCGGGATTGAGCGATGG | 36-52 |
| 104) | AAC (SEQ ID NO: 149) | |
| | GTTCCATCGCTCAATCCCGATGGACAACCACTGC | |
| | AAC (SEQ ID NO: 203) | |
| 3300014158\|Ga0181521_10019591_ | GTTTCAATATTAGGTCCAGCCGGAGTCAGGCTGG | 35-37 |
| 3\|P (SEQ ID NO: 105) | AAC (SEQ ID NO: 150) | |
| | GTTCCAGCCTGACTCCGGCTGGACCTAATATTGA | |
| | AAC (SEQ ID NO: 204) | |
| 3300014159\|Ga0181530_10005765_ | GTTTCAATATTAGGTCCAGCCGGAGTCAGGCTGG | 35-37 |
| 11\|M (SEQ ID NO: 106) | AAC (SEQ ID NO: 150) | |
| | GTTCCAGCCTGACTCCGGCTGGACCTAATATTGA | |
| | AAC (SEQ ID NO: 204) | |
| 3300014638\|Ga0181536_10002122_ | GTTTCAATATTAGGTCCAGCCGGAGTCAGGCTGG | 35-37 |
| 15\|P (SEQ ID NO: 107) | AAC (SEQ ID NO: 150) | |
| | GTTCCAGCCTGACTCCGGCTGGACCTAATATTGA | |
| | AAC (SEQ ID NO: 204) | |
| 3300009632\|Ga0116102_1009001_2\| | GTTTCAACATTAGGTCCAGCCGGAGTCAGGCTGG | 29-37 |
| P (SEQ ID NO: 108) | AAC (SEQ ID NO: 151) | |
| | GTTCCAGCCTGACTCCGGCTGGACCTAATGTTGA | |
| | AAC (SEQ ID NO: 205) | |
| 3300009643\|Ga0116110_1007258_2\| | GTTTCAACATTAGGTCCAGCCGGAGTCAGGCTGG | 29-37 |
| M (SEQ ID NO: 109) | AAC (SEQ ID NO: 151) | |
| | GTTCCAGCCTGACTCCGGCTGGACCTAATGTTGA | |
| | AAC (SEQ ID NO: 205) | |
| 3300009643\|Ga0116110_1007258_2\| | GTTTCAACATTAGGTCCAGCCGGAGTCAGGCTGG | 29-37 |
| P (SEQ ID NO: 110) | AAC (SEQ ID NO: 151) | |
| | GTTCCAGCCTGACTCCGGCTGGACCTAATGTTGA | |
| | AAC (SEQ ID NO: 205) | |
| 3300017996\|Ga0187891_1001667_2 | GTTTCAATATTAGGTCCAGCCGGAGTCAGGCTGG | 35-37 |
| 3\|M (SEQ ID NO: 111) | AAC (SEQ ID NO: 150) | |
| | GTTCCAGCCTGACTCCGGCTGGACCTAATATTGA | |
| | AAC (SEQ ID NO: 204) | |
| 3300018003\|Ga0187876_1014226_2\| | GTTTCAATATTAGGTCCAGCCGGAGTCAGGCTGG | 35-37 |
| P (SEQ ID NO: 112) | AAC (SEQ ID NO: 150) | |
| | GTTCCAGCCTGACTCCGGCTGGACCTAATATTGA | |
| | AAC (SEQ ID NO: 204) | |
| 3300025453\|Ga0208455_1001030_8\| | GTTTCAACATTAGGTCCAGCCGGAGTCAGGCTGG | 29-37 |
| P (SEQ ID NO: 113) | AAC (SEQ ID NO: 151) | |
| | GTTCCAGCCTGACTCCGGCTGGACCTAATGTTGA | |
| | AAC (SEQ ID NO: 205) | |
| 3300025507\|Ga020818 8_1002252_2\| | GTTTCAACATTAGGTCCAGCCGGAGTCAGGCTGG | 29-37 |
| M(SEQ ID NO: 114) | AAC (SEQ ID NO: 151) | |
| | GTTCCAGCCTGACTCCGGCTGGACCTAATGTTGA | |
| | AAC (SEQ ID NO: 205) | |
| 3300025507\|Ga020818 8_1002252_2\| | GTTTCAACATTAGGTCCAGCCGGAGTCAGGCTGG | 29-37 |
| P (SEQ ID NO: 115) | AAC (SEQ ID NO: 151) | |
| | GTTCCAGCCTGACTCCGGCTGGACCTAATGTTGA | |
| | AAC (SEQ ID NO: 205) | |
| PJQE01000204_l\|M (SEQ ID NO: | TTCGGATCATCCGAAGATTT (SEQ ID NO: | 37-43 |
| 116) | 152) | |
| | AAATCTTCGGATGATCCGAA (SEQ ID NO: | |
| | 206) | |
| 3300006845\|Ga0075421_100006223 | GTGACTGCAGACTGGTCGTGCGAATGCGCACGGC | 34-35 |
| _8\|P(SEQID NO: 117) | AC (SEQ ID NO: 153) | |
| | GTGCCGTGCGCATTCGCACGACCAGTCTGCAGTC | |
| | AC (SEQ ID NO: 207) | |
| 3300006847\|Ga0075431_100017297 | GTGACTGCAGACTGGTCGTGCGAATGCGCAC | 39-40 |
| _1\|M (SEQ ID NO: 118) | (SEQ ID NO: 154) | |
| | GTGCGCATTCGCACGACCAGTCTGCAGTCAC | |
| | (SEQ ID NO: 208) | |
| 3300027909\|Ga0209382_10008589_ | GTGACTGCAGACTGGTCGTGCGAATGCGCACGGC | 34-35 |
| 8\|P(SEQ ID NO: 119) | AC (SEQ ID NO: 153) | |
| | GTGCCGTGCGCATTCGCACGACCAGTCTGCAGTC | |
| | AC (SEQ ID NO: 207) | |
| ODAK010035847_2\|M (SEQ ID NO: | GTTGCAGATATTGGTCCAGCCGGGGTTGGGCTGG | 36-38 |
| 120) | AAC (SEQ ID NO: 155) | |
| | GTTCCAGCCCAACCCCGGCTGGACCAATATCTGC | |
| | AAC (SEQ ID NO: 209) | |
| SRR6266511_1572288_18\|M (SEQ | AGTGATTGAAGGCTGGTCGTGCGAATGCGCACGG | 33-35 |
| ID NO: 121) | CAC (SEQ ID NO: 156) | |
| | GTGCCGTGCGCATTCGCACGACCAGCCTTCAATC | |
| | ACT (SEQ ID NO: 210) | |
| SRR6266511_203987_6\|P (SEQ ID | GCCGTGCGAATGCGCACGGCATG (SEQ ID | 33-51 |
| NO: 122) | NO: 157) | |
| | CATGCCGTGCGCATTCGCACGGC (SEQ ID | |
| | NO: 211) | |
| SRR6266511_239355_1\|M (SEQ ID | GTAATTGAAAGTTGGTCGTGCGAATCCGCACGGC | 26-40 |
| NO: 123) | AC (SEQ ID NO: 158) | |
| | GTGCCGTGCGGATTCGCACGACCAACTTTCAATT | |
| | AC (SEQ ID NO: 180) | |
| SRR6266511_838544_1\|M (SEQ ID | GTAATTGAAAGTTGGTCGTGCGAATCCGCACGGC | 34 -36 |
| NO: 124) | AC (SEQ ID NO: 158) | |
| | GTGCCGTGCGGATTCGCACGACCAACTTTCAATT | |
| | AC (SEQ ID NO: 180) | |
| 3300014502\|Ga0182021_10233972_ | GTTGCAGTGGTTGTCCATCGGGATTGAGCGATGG | 36 |
| 2\|M (SEQ ID NO: 125) | AAC (SEQ ID NO: 149) | |
| | GTTCCATCGCTCAATCCCGATGGACAACCACTGC | |
| | AAC (SEQ ID NO: 203) | |
| 3300005529\|Ga0070741_10001368_ | GTGACAACTCTTGCTCAACCGAGTCTCGGTTGAG | 36 |
| 72\|P (SEQ ID NO: 126) | AC (SEQ ID NO: 159) | |
| | GTCTCAACCGAGACTCGGTTGAGCAAGAGTTGTC | |
| | AC (SEQ ID NO: 212) | |
| 3300010046\|Ga0126384_10047357_ | GTGATTGAAGGCTGGTCGTGCGAATGCGCACGGC | 34-37 |
| 3\|M (SEQ ID NO: 127) | AC (SEQ ID NO: 160) | |
| | GTGCCGTGCGCATTCGCACGACCAGCCTTCAATC | |
| | AC (SEQ ID NO: 213) | |
| 3300010359\|Ga0126376_10030051_ | GTGATTGAAGGCTGGTCGTGCGAATGCGCACGGC | 35-37 |
| 2\|M (SEQ ID NO: 128) | AC (SEQ ID NO: 160) | |
| | GTGCCGTGCGCATTCGCACGACCAGCCTTCAATC | |
| | AC (SEQ ID NO: 213) | |
| 3300010362\|Ga0126377_101l1069_ | GTGATTGAAGGCTGGTCGTGCGAATGCGCACGGC | 35-37 |
| 2\|M (SEQ ID NO: 129) | AC (SEQ ID NO: 160) | |
| | GTGCCGTGCGCATTCGCACGACCAGCCTTCAATC | |
| | AC (SEQ ID NO: 213) | |
| 3300012204\|Ga0137374_10002290_ | GCCGTGCGAATGCGCACGGCATG (SEQ ID | 33-51 |
| 4\|M (SEQ ID NO: 130) | NO: 157) | |
| | CATGCCGTGCGCATTCGCACGGC (SEQ ID | |
| | NO: 211) | |
| 3300012204\|Ga0137374_10009879_ | AGTGATTGAAGGCTGGTCGTGCGAATGCGCACGG | 33-35 |
| 6\|P(SEQID NO: 131) | CAC (SEQ ID NO: 156) | |
| | GTGCCGTGCGCATTCGCACGACCAGCCTTCAATC | |
| | ACT (SEQ ID NO: 210) | |
| 3300012350\|Ga0137372_10022207_ | AGTGATTGAAGGCTGGTCGTGCGAATGCGCACGG | 33-35 |
| 3\|M (SEQ ID NO: 132) | CAC (SEQ ID NO: 156) | |
| | GTGCCGTGCGCATTCGCACGACCAGCCTTCAATC | |
| | ACT (SEQ ID NO: 210) | |
| 3300012350\|Ga0137372_10022207_ | AGTGATTGAAGGCTGGTCGTGCGAATGCGCACGG | 33-35 |
| 4\|P(SEQID NO: 133) | CAC (SEQ ID NO: 156) | |
| | GTGCCGTGCGCATTCGCACGACCAGCCTTCAATC | |
| | ACT (SEQ ID NO: 210) | |
| 3300012353\|Ga0137367_10000906_ | GCCGTGCGAATGCGCACGGCATG (SEQ ID | 33-51 |
| 4\|M (SEQ ID NO: 134) | NO: 157) | |
| | CATGCCGTGCGCATTCGCACGGC (SEQ ID | |
| | NO: 211) | |
| 3300012353\|Ga0137367_10004593_ | AGTGATTGAAGGCTGGTCGTGCGAATGCGCACGG | 33-35 |
| 5\|M (SEQ ID NO: 135) | CAC (SEQ ID NO: 156) | |
| | GTGCCGTGCGCATTCGCACGACCAGCCTTCAATC | |
| | ACT (SEQ ID NO: 210) | |
| 3300012355\|Ga0137369_10001944_ | GCCGTGCGAATGCGCACGGCATG (SEQ ID | 33-51 |
| 2\|P(SEQID NO: 136) | NO: 157) | |
| | CATGCCGTGCGCATTCGCACGGC (SEQ ID | |
| | NO: 211) | |
| 3300012355\|Ga0137369_10007378_ | AGTGATTGAAGGCTGGTCGTGCGAATGCGCACGG | 34-35 |
| 6\|M (SEQ ID NO: 137) | CAC (SEQ ID NO: 156) | |
| | GTGCCGTGCGCATTCGCACGACCAGCCTTCAATC | |
| | ACT (SEQ ID NO: 210) | |
| 3300012355\|Ga0137369_10043960_ | AGTAATTGAAAGTTGGTCGTGCGAATCCGCACGG | 33-36 |
| UM (SEQ ID NO: 138) | CAC (SEQ ID NO: 148) | |
| | GTGCCGTGCGGATTCGCACGACCAACTTTCAATT | |
| | ACT (SEQ ID NO: 202) | |
| 3300012358\|Ga0137368_10001884_ | GCCGTGCGAATGCGCACGGCATG (SEQ ID | 33-51 |
| 4\|M (SEQ ID NO: 139) | NO: 157) | |
| | CATGCCGTGCGCATTCGCACGGC (SEQ ID | |
| | NO: 211) | |
| 3300012358\|Ga0137368_10007571_ | AGTGATTGAAGGCTGGTCGTGCGAATGCGCACGG | 33-35 |
| 6\|P (SEQ ID NO: 140) | CAC (SEQ ID NO: 156) | |
| | GTGCCGTGCGCATTCGCACGACCAGCCTTCAATC | |
| | ACT (SEQ ID NO: 210) | |
| 3300012360\|Ga0137375_10064112_ | GCCGTGCGAATGCGCACGGCATG (SEQ ID | 33-51 |
| UP (SEQ ID NO: 141) | NO: 157) | |
| | CATGCCGTGCGCATTCGCACGGC (SEQ ID | |
| | NO: 211) | |
| 3300012532\|Ga0137373_10033017_ | GCCGTGCGAATGCGCACGGCATG (SEQ ID | 33-51 |
| UP (SEQ ID NO: 142) | NO: 157) | |
| | CATGCCGTGCGCATTCGCACGGC (SEQ ID | |
| | NO: 211) | |
| 3300012944\|Ga0137410_10000075_ | CGTAGCAATTGATGTTCAATGGATGAGCC (SEQ | 44 |
| 50\|P (SEQ ID NO: 143) | ID NO: 161) | |
| | GGCTCATCCATTGAACATCAATTGCTACG (SEQ | |
| | ID NO: 214) | |
| 3300015245\|Ga0137409_10000164_ | CGTAGCAATTGATGTTCAATGGATGAGCC (SEQ | 44 |
| 50\|P (SEQ ID NO: 144) | ID NO: 161) | |
| | GGCTCATCCATTGAACATCAATTGCTACG (SEQ | |
| | ID NO: 214) | |
| SRR6837571_912213_1\|M (SEQ ID | GTCGCGAATAACTGTTCAGCGGAGAAGCCGCTGA | 33-34 |
| NO: 145) | AAC (SEQ ID NO: 162) | |
| | GTTTCAGCGGCTTCTCCGCTGAACAGTTATTCGC | |
| | GAC (SEQ ID NO: 215) | |

TABLE 12
Non-coding Sequences of Representative
CLUST.099129 Systems

>SRR6837557_1470862_2|M
ACGAAGGGTTGGCCACGTTTGTGGAGCCGTGGCCGGGCGATGA
TGTGGCCGTATACGTGAAAGATGAAGCCGAGAATGTGAAACAG
GTGTTGGCGGTGACCAAGGCCGCCGGGCTGACGCCGATTGGGT
TTGTGAAAACGGAAGGGCTGTAATCTCGCCTTGATATTGGCTT
AATTTCAGGTTGAAAGTAAGAACGGGACACATAATGAGAACAA
CCAAAACCCCTGATTATTCCAATAAAAATCTGTCCTTCTGGTG
TGAGCCGGTCGGCGAAATGCCGAGTGTGGTTTTTGAAAAGGGT
CGGCAGATGAACCGGCTCTGGAATCTGCTGGTTGAGTGTCACA
CTACGTTTCTCCACCGACGGGATCAAGACAAGGGTTCCGCGAA
AATGCTATTTGAAGAGATTGACGAAGGTTATCGGCAGCAATCG
GCCCCGCTAAGCCTGGATTTGTTTACGGATCAGGCGGTTCGCG
CCATTCGCACCTTAAATCGCGAGATGGTTTGAGTTATAGGGCT
GACTACTCTC
(SEQ ID NO: 163)
>SRR6266511_239355_1|M
AAACAGTGGCGAGAGCCATATTATTGGGCCGCCTTCGTTCTGC
AGGGTGAATGGAAATAGCGATGGGCGCGTCAATTTCTCGGAAG
AAGGGATGGGGTGTCGCGCGACATAGATGAAAATGCAATGATG
CGGCAGGAGTTCAGCAAAGTGCAGGCGCAATCCGGAGGCATAG
ACAGAAGGCCCCTTCCGTGATTACATTAGTCTTCGCGCCTTCG
ATTGCCGGTTATAAACCGATAGCTCCCTTTCGCAAAATTTATG
GAAGATATATCCAAGAAGCCGGAGCGGCGTAAAAACTTCGCGC
GCCGAGAACGGCTGAACAAAGATTGGCCGTTTAAGGTCTACCC
CTACAGATGCATGCTGCGGGAGAAACCGCCCCAAGCGCTGTGG
GACACAGCGCGTGAGATGGACGTGACCACAAGCCTATACCTGC
TGAGTAAGATAGAAGGCTACGCAAGCGCCAGTGGCCCTCCTAT
TGAAATTCCGGCGCATCTGAGCCAATACTTGCGAGTGATGCAC
GCAAGTGAAGTGCGGCTTGAGATTGTTGAAAAGCATTGACACT
CCCACCGCTGGAAGCGGCGGGATTCTTGCTTCAACGCGAACGG
CCTGCCGTGGCAGGTCTTACACTCGCTCCACAAGCGTTTTGAC
TCTCCGCGTGCCCCGCGGCGAGGAGAATGTTGATTCCTTCGTT
TCTGATGTTTTTGGCCGCGTTACGATCTCGATCATGCCGCGCG
CCGCAATTGACGGATTTGCCTGTTTGTTGATAGGCCTCCTTTC
GGCGCGCCAACGCCCAGTTGAAGACGAATCTGCGCGCGCCCGC
GAAACGGGCGAAAGCCGTCTCCTCGTCGGCGGTCGGATCGAGC
CTGAATCTGTAGACCTTTCGTTCCGGCATTCAATCTCTAGTGC
CCTGCGAGTTGATGTATTCGCCAAATCCACGTCGTGGGCGGGC
ATCGGGGAGTCAATGGCGTTACGCGTTGCTTCGCGCCGCGCCT
GTCATCCCCCCGCTGGAAGCGGAGGGCTTTCTTTGGCTTTTTT
TGAAAGTGGCTAAGGTGCCCATCACAGTTCGAAAAAGTAATTG
AAATGGTGATCTATGTTCAATCAAACCGTACCAGTCGCAAACC
CGCAGGGTAGGCGGGGAGCGACTGCCCGCCACACCGTTCAGAT
TATCAACCTGATCCTTTGGGCTGGATTGTCAGTTTCTCCATGC
CTGTGGCCAGGGGCGGAGCCTGCGCAAGCCTCC
(SEQ ID NO: 164)
>SRR6837571_912213_1|M
GAAGGGTTGGCCACGTTAGTGGAGCCGTGGCCGGGCGATGATG
TGGCCGTATACGTGAAAGATGAAGCCGAGAATGTGAAACAGGT
GTTGGCGGTGACCAAGGCCGCCGGGCTGACGCCGATTGGGTTT
GTGAAAACGGAAGGGCTGTAATCTCGCCTTGATATTGGCTTAA
TTTCAGGTTGAAAGTAAGAACGGGACACATAATGAGAACAACC
AAAACCCCTGATTATTCCAATAAAAATCTGTCCTTCTGGTGTG
AGCCGGTCGGCGAAATGCCGAGTGTGGTTTTTGAAAAGGGTCG
GCAGATGAACCGGCTCTGGAATCTGCTGGTTGAGTGTCACACT
ACGTTTCTCCACCGACGGGATCAAGACAAGGGTTCCGCGAAAA
TGCTATTTGAAGAGATTGACGAAGGTTATCGGCAGCAATCGGC
CCCGCTAAGCCTGGATTTGTTTACGGATCAGGCGGTTCGCGCC
ATTCGCACCTTAAATCGCGAGATGGTTTGAGTTATAGGGCTGG
CAGAGCCGTTGAAATACTCTCGTCAAAGATCAAAAGTTAGTTT
GGCAATAAATGTGGCTTTACTTTATAACCTAGTAGTGTTATAG
TTCACCCGTTGTTGGATGTGCTTAACTTTTACAACTTTGGTG
(SEQ ID NO: 165)
>SRR6266511_838544_1|M
AAACAGTGGCGAGAGCCATATTATTGGGCCGCCTTCGTTCTGC
AGGGTGAATGGAAATAGCGATGGGCGCGTCAATTTCTCGGAAG
AAGGGATGGGGTGTCGCGCGACATAGATGAAAATGCAATGATG
CGGCAGGAGTTCAGCAAAGTGCAGGCGCAATCCGGAGGCATAG
ACAGAGAGCCCCTTCCGTGATTACATTAGTCTTCGCGCCTTCG
ATTGTCGGTTATTAACCGATAGCTCCCTTTCGCAAAATTTATG
GAAGATATATCCAAGAAGCCGGAGCGGCGTAAAAACCCCGCGC
GCCGAGAGCGACTGAACAAAGATTGGCCAATTAAGGTCCATAA
CTATAGATGCTCGCTGAATGAGAAACCGCCCCAGACGCTCTGG
GATACGGCGCACGAGATGGATCAGGACGTGACCACAAGCCTAT
ACCTGCTGAGTAAGATAGAAGGCTGCGCAAGCGCCAGTGGCCC
TCCTATTGAAATTCCAGCGCATCTGAGCCCATACTTGCGAGTG
ATGACCGCAAGTGAAGTGGCTCTTGAGATTGTTGAATAGCATG
AAAGTGGCTAAAGTACCCATCAAAGTTCGAAAAAGTAATTGAA
ATGGTGATCTATGTTCAATCAAATCGTACCAGTCGCAAACCCG
CAGGGTAGGCGGGGAGCGACTGCCCGCCACACCGTTCAAATTA
TCAACCTGATCCTTTGGGCTGGATTGTCAGTTTCTCCATGCCT
GTGGCCAGGGGCGGAGCCTGCGCAAGCCTCCGCTAGTGAACGC
GGCGCGGCGCGCCGCGTTCAGGAGAGTATTTCGCTGGAACCGG
GCAAGCCGATCGAGCGAGAGCCCTCCGGCGGCGAGTCGCATTC
CTAC
(SEQ ID NO: 166)
>3300025507|Ga0208188_1002252_2|M
GCAACGATGTTCTTTGTATATGCCAGGCTGTTCTTTTCTCCGG
CCGCTTCTTTGGGGCAGACAAGCAAAATGGTCGCGTTGGATGA
CCTGAGGTCAATTAGAGAATTGTCCCTAGCGAGATCCAGGAGC
CGTTCGAGCTTCTTAACCAAACTGCCCGTACTTCTCACAGAGC
GGTTTGGGATCGGACGCCGCGCAAGGTATCGCCTCATGTAATC
GGGGTTCGCTTTACGCCATTTCCGCTGGCTGCGACGGCACTGT
TCCTTGTAGGCAGGATCGGTGTCGAGTTTCCATCGGTGATAGT
CCGCGCGGCGTCGACGTTGGCATTCGCCGGAGCCGCAGACTTT
CTGATTAGGATGATAGCGGGATGGCTTAAATCTTCGTTTGCAG
TAAGGACATGTCTTTTGTCGCATACGATTTGTGGAAGCAGTCT
GTGTTAGAACTACTTCGGAGGGGAGAGCGAGGCGCCAATTAGA
CGTTCACTCAGCAACCGCGCCCTGTATAATGAGAGCGGAATGG
GATATTGATAATCGTGCTCAGCGTCCTAGGACAGGTGGGCTGG
ACACCGTCCCGGCGCTTGAGTGAGTGGTGAAGATAGCTCACGG
CATCGAAGAATGTCCGCAAGGACGATAGAGGAAAGAAAAGGCA
AAACCAGCATGCAGAGAGAGCGCAAGCGCAAAAAGACCCACGA
CATAAGGCCACATAAGTACCGCGCATGGTTCGCCGAAGGTGAT
GGAACCCGGCTGCCGGAGCCCATCTTTGCGACCTGCGCGGCTT
GCGGCTCGCATTTCGAAGCGGGGGCCGGGCTCATGGGGCGCTG
CGAGCAGGGGCACGTGATGGACCAGGACTGGAATGCGGCCGGG
AACATCTACGCAGGCCGAGCGGGCGCCACCGCGCAAGCGGCAG
TCGGGTAAGAATGGGGGCGGCACGAGGAGGCGACCAGGTGCGT
TTGCATCCCGCGTGGGGGGTCGGCACATCAGAGTCCAGCCGTA
CGCAGGCTGGGGCTAGCAACTGATGCACTGGGGGGTAAGAACG
GGTATTTGCGAAAAGGGTCATGCTACGGGCCAGGACCTTGACG
GAGCAGAGAGCAACTACGCAAACCGGGGCGGGGCTGCCGCCGC
CGCGGGGTTTGCGTAAAAGCTGTGCCGACAAATGCGCCAAATA
TATGTTAGTGTTGTGGGTTGTGGAGCGGTTCGTGCTTCCCTCG
CGGCGGGAATCGGTCAGATGCTGCGGGATCAATTATCCGTTTT
CGGATCTGCTTGACGCACGCGCTCCGAAAGCTCACCATTTACA
CAAGACCTCGACGGGATCGGTCTGGCAAGCGACGACCCTGACA
CCTGCGGAGATGGCGTCGATCAGCTACTCCGATTCAGAGCAAT
CCAGTAGCAGAATGGAACGTGTGGTTGGGGCAGAAGCGCGCAG
CTCTCGAACGACCTTAACACCCCCTGGGGACCACCTTCCAGAT
TCGAGCTGATCACAGCTGCATTCGGGCGATACTCGGCTACTTG
CTCAAAAACTCCTTTGGTGTGTGTTTAGCCCCCAACAACCAAG
TGCTTGCGCTGCTTTTTGAGCGCACGGCACAGCACCTTGCCGA
CCAGCGCTGTGCGTTGGGCGACGACAACTGATATAGGCACGGG
CGTGATTCCCTCCTAACCCGAAGTTCCAAGAGGCTGCAGAGAG
TATTCTACTGATTCACTGAGAGATAGTAGCAAACTCAATTTCC
CGTTACTGGGAACGTACGAATCAGTTCGCGCGCCCGCGCTTGT
AGGGGTGTGGGAGTCGGCACCTGTTGGCAGGTCAGGCTCGCGT
CTCCCGATTTGATCTGATAGGTGACCCTCGCCCGGCTGGCCAA
CTCGGCGAGCAG
(SEQ ID NO: 167)
>3300014158|Ga018152l_10019591_3|P
AACCAATTGAAGGCTGCATAGAATAGTGGCCAGCGCCATCTTC
TCAAAACGGCAATTCAGCTTGCTCGCCGCAGCAGGCGACAATC
GCTTGCTGCCGAAATGTTGAGAGGCCTCGCGAATGTGGGCTGC
GAGATTACGTATGCTTTTCATTTTGTTTGGTTCGGACTAGACT
CAATATCCTATGCAAAGGGAGCGCAAGCGCAAGGAGAGCCACG
ACATAAGGCCACACAAGTACCGGGCGTGGTTCGCCGAGGGCGA
CGGGACCAGGCTTCCCGAGCCTGTCTATTTGCTTGCAAAGCGC
ATGCAGGATTGCTGGACGGACATTGCGCTCACATGCGCGGCTT
GCGGCTCGCATTTCGAGGCGGGGGCCGGGCTCATGGGGCGCTG
CGAGCAGGGGCACGTGATGGACCAGGACTGGAATGCGGCCGGG
AACATCTACGCAGGCCGAGCGGGCGCCACGGCGCAAGCGGCAG
TCGGGTAAGAATGGGGGCGGCGCGAGGAGGCGACCAGGTGCGT
TTGCATCCGGCGTGGAGGGTCGGCACATCAGAGTCCAGCCGCA
CCCAGGCTGGGGCAAGCAACTGATGCACAGGGGTGTAAGAACG
GGTACTTGCGAAAAGGGTCATGCTATGGGCCAGGACCTTGACG
GAGCAGAGAGCAATTGCGCAAACCGGGGCGGGGCTGCCGCCGC
CGCGGGGTTTGCGTAAAATCTGTGCCGACAAATGCGCCAAATA
TATGTTAGTGTTGTGGGTTGTGGAGCGCTTCATGGAAATGAAT
CACTGCGAATCTCTTCGCAAGATTCAAATACTGAACTGATTAA
ACGTCAGCTACGGGCTGTCCGATAGTATGGCGCAATGATACCG
GTGATGGAGATGTTACAATCTGCGTGAATAGTGGGGACGAAGT
ACAAGTAGACGGCCCCGGCGGCAGTACGTCAATTGGAACCGAT
CACGTTCTGCCCCCCATCGCCGCAAAGGGATCATGATAAAGTG
TCGTCATTTTGCAGCCTGCGGCGCCCCGCGACCGCGTTCAGAT
CCCGTGATTCGACCTCCAATGGCATGCTTCTCCTTGGGCCCTG
CGCGACTGTAGCTTCCGACTTGTTCACTATCCTCCTGCTTCGT
CTGACCCTCGCTCGATTTTACTTTTATGGTAAGATTGCCTCCA
TCTGGCCGCTGGGGCTCTGGTGGAGATCGTGTGATTACGGCAA
AGGACACGAGCAATCCAGTAGGAGGATGGAACGCGTAGTTGGG
GCAGAAGCGCGCAGCTCTCGAACGACCTTAACACCCGCCTGGG
GATTACCTTCCAGATTCGAGCTGATCACAGCTACATTCGGTTG
ATACTCAACTACGTGCTCAAAAAACTTCTTTGGTGTGTGTTCA
GCCCACAACAACCAAGCGCCTGCGCTGCTCTTTGAGCGCACGG
ACCAGCACCTTGCCGACCAGCGCTGTGCGTTGGGCGACGATAA
CTGATATAGGCCAGGGCGTGATTCCTTCCTAACCGCTCCGGTA
TTGTCCGGGGTAGCCCGCTGATCGCTGAACAGAAGTCTCGTAG
CCTCATTTAGCAGGAGCAGTTCGCTGGTCGGCCATCACCACGC
GCCCCTCGCCGACTGAGAGGATGCTATCCGCCAACTCCCAGAC
AAGTGCTGTATGGCAGATAAAGATCATTTGGTGGAATCCGCCA
AGATCCATGGCACGGCGCAGCAT
(SEQ ID NO: 168)
>3300014502|Ga0182021_10233972_2|M
AGAGGCTGACACCGCACCGCAGCAGCCTCAACTGCCCGTCTGG
TTGACGGCGCTGAACCCCAGGCGCATCACTGCCTGGGTCTACG
AACTGAGTAACGGAGTACGCTTCGAAGCACGCGGCGGCGGATG
GGTGCAGCAAGGCGGGAGAAATTCGGAGTGGGTCTATACCACC
ACGAACGGTCCGCGAGATTTCGGTGTCGCTGGTTAACAACGGC
TTCTACTCGACCGGGCGACGGGAAAGTTGTAACACGCGCGCCA
GTGGGATATTCGTTTCTATAGAAACACTGTGAGCGATGTGACG
TTTTATTTCGAATTGATTAGAAGCCTGTTGGAGCAGCAATGAA
ACCTAAAAAGAACGCCGACACGATCTACCAAACCTACAAATTC
TGGATCAAGCCGAGCGACCCCCTGCCTCAATTATTGTGGGACA
AAGCGAAGGCCATGAATGCCGCGTGGACGAGTTTAGTCCTCAT
GCGTCAGGAGACGCTGGTGAGAAACCTAACC
(SEQ ID NO:
169)
>3300005764|Ga0O66903_100343966_1|M
CGGTTATAAACCGATAGCTCCCTTTCGCAAAATTTATGGAAGA
TATATCCAAGAAGCCGGAGCGGCGTAAAAACCCCGCGCGCCGA
GAACGACTGAACAAAGATTGGCCAATTAAGGTCCATAACTATA
GGTGCTCGCCGAATGAGAAACCGCCCCAGGCGCTCTGGGACAC
GGCGCACGAAATGGATCAGGATGTAACTGCAAGTCTGTACTTG
CTGAGTAAGATAGAAGGCTACGCAAGCATCAGTGGCCCTCCTA
TTGAAATTCCGGCGCATCTGAGCCCTTACTTGCGGGTGATGAC
CGCAAGTGATGTGGCTCTTGAGATTGTTGAATAGCTGAAAGTG
GCTAAGGTAGTCGCACGTGAAAAAGTGTAAAAGCCAATAAAAA
CGGCATGTTCATCGAGAATCACACCGTGTAGAATTGCAAGAAA
GCAGTAGAAACCT
(SEQ ID NO: 170)
>3300009643|Ga0116110_1007258_2|M
CGGAGGGGAGAGCGAGGCGCCAATTAGACGTTCACTCAGCAAC
CGCGCCCTGTATAATGAGAGCGGAATGGGATATTGATAATCGT
GCTCAGCGTCCTAGGACAGGTGGGCTGGACACCGTCCCGGCGC
TTGAGTGAGTGGTGAAGATAGCTCACGGCATCGAAGAATGTCC
GCAAGGACGATAGAGGAAAGAAAAGGCAAAACCAGCATGCAGA
GAGAGCGCAAGCGCAAAGAGACTCACGACATAAGGCCACATAA
GTACCGCGCATGGTTCGCCGAAGGTGATGGAACCCGGCTGCCG
GAGCCCATCTTTGCGACCTGCGCGGCTTGCGGCTCGCATTTCG
AAGCGGGGGCCGGGCTCATGGGGCGCTGCGAGCAGGGGCACGT
GATGGACCAGGACTGGAATGCGGCCGGGAACATCTACGCAGGC
CGAGCGGGCGCCACCGCGCAAGCGGCAGTCGGGTAAGAATGGG
GGCGGCACGAGGAGGCGACCAGGTGCGTTTGCATCCCGCGTGG
GGGGTCGGCACATCAGAGTCCAGCCGTACGCAGGCTGGGGCTA
GCAACTGATGCACTGGGGGGTAAGAACGGGTATTTGCGAAAAG
GGTCATGCTACGGGCCAGGACCTTGACGGAGCAGAGAGCAACT
ACGCAAACCGGGGCGGGGCTGCCGCCGCCGCGGGGTTTGCGTA
AAAGCTGTGCCGACAAATGCGCCAAATATATGTTAGTGTTGTG
GGTTGTGGAGCGGTTCGTGCTTCCCTCGCGGCGGGAATCGGTC
AGATGCTGCGGGATCAATTATCCGTTTTCGGATCTGCTTGACG
CACGCGCTCCGAAAGCTCACCATTTACACAAGACCTCGACGGG
ATCGGTCTGGCAAGCGACGACCCTGACACCTGCGGAGATGGCG
TCGATCAGCTACTCCGATTCAGAGCAATCCAGTAGCAGAATGG
AACGTGTGGTTGGGGCAGAAGCGCGCAGCTCTCGAACGACCTT
AACACCCCCTGGGGACCACCTTCCAGATTCGAGCTGATCACAG
CTGCATTCGGGCGATACTCGGCTACTTGCTCAAAAACTCCTTT
GGTGTGTGTTTAGCCCCCAACAACCAAGTGCTTGCGCTGCTTT
TTGAGCGCACGGCACAGCACCTTGCCGACCAGCGCTGTGCGTT
GGGCGACGACAACTGATATAGGCACGGGCGTGATTCCCTCCTA
ACCCGAAGTTCCAAGAGGCTGCAGAGAGTATTCTACTGATTCA
CTGAGAGATAGTAGCAAACTCAATTTCCCGTTACTGGGAACGT
ACGAATCAGTTCGCGCGCCCGCGCTTGTAGGGGTGTGGGAGTC
GGCACCTGTTGGCAGGTCAGGCTCGCGTCTCCCGATTTGATCT
GATAGGTGACCCTCGCCCGGCTGGCCAACTCGGCGAGCAG
(SEQ ID NO: 171)
>ODAK010035847_2|M
CCTCTTCCCTTAACTGTTTTCCTCCCGTGGCGGATTCCGTTCT
GATGCCATCGAGTTTTTCGCCACTGCTCTTCGTCAATGAGACG
ATCTGCCCGGAGAATTTTTCAAGTTGAATTTTCTCGGCACCCG
CCAAATCTTTAATTGTCTTGGACATCGTTTCGGAAATACTGTT
GAGCGTGGTAACAACTTCTTCTCGAAGGTGCTGGGCACTAGCG
GCGGACTCGGTACGACCACAGTCGAGCCTTTCGACGCTGGTTT
GGGCAAAGGCGGCAAGCTGGGTTGCAAATCCTTCAAACTGTGA
CTTTTGGGCTGAAGCCAGACCGCTTATCGTTTTTGTTATCGTC
TCGGAAATGGTGCTGAGAGTCGCTACTACTTCTTCCCGTAGTT
GCTTGGCTCCCGTGGCGGATTCGGTGCGAACACCATCCAATCG
CGCACCGCTCGCTTTAGCGAACGAGGCAAGCTGCTCAGAGAAA
GCGTCAAGTTGGCTTTTCTGGACATTAGCCCACTCCCGCATCG
TCCCCGTCGTCGTCTCCGAGATGCTAGTCAGCGTTGCCACGAC
TTCCTGCCGCTGCTCCTTCGCGGCCTTACCCAGTTCGACCCTA
CTCTGGGCAACTTCCTCACCCAGTTCGTCCCTACTCTGGGCAA
CTTCCTCTCTGACTGCACGCTCGGTGCGTTCTTGAGCCTTTTC
AAAAGCATCCAGACGGGAATCGAGCATTGGCGAGCCGACTTTG
GATGTTTTCATCAAAAGGGCAAGGAGAAGAGCCACCACAACGA
CCAGCAGGACGAGAGTCACGACAGGCATAAATTTGAAGTCTTT
CCGAGTAGGGACGATTATCGAACCCACTCATCAGGGTCGGGGG
TTCAAGCCGCAGCCAGTTGTCTCAGTCTGGGTTCCCTTGGAGT
GTTGGCTGAGGTTCAAGTCGCCGGGGAAGAAAATGCGACCAGT
ATGTAACCTTAACGCAAGAACCTACCGAAACTCGTGACCCAAA
TCACAGTCTGTGGTGACTGGAAACACCAAGGCCCCAAACACTC
CGGAGGCAATCAAAGTGGCAGCACGCGGGTCACCCTTTCGCTG
GCGGAACGTGATGCCCAAGATCGGCGTATCGCCGCCGTCGCTG
AAACACTAGGAGGGAAGGCTTTTTGAATTAGGGACGAGAAATC
CACATTAAATTCACATTGAAATCGTGCTACATTTCGGCGGATA
TGTAGAGCCGAGTGACGGCTTTCGGCTGGAAACGTATACTACG
ATTATGCGAGAGCGTAAGCGCCAAGAGACGCACGGACAGATGG
CTTATAAGTTTCGTGCCTACGTCACCGGAACAGACGCCAGCCA
CGGGCAGATGTTGCCCCCGGCGATTTACACCGTCGCTAAGAAC
ATGGAGAACTGCTGGTCGGACATCGCCAATCATAACGAGCAAG
AATACGGGGCTTGGAAGATCTGTTCCGAATGTGGGGCTTTGTT
TGAAGCAGGTAGCGAGATTATAGGACGTTGCACGCGGGGGCAC
GAAAAAGACCAAGATTGGAATGCAGCCGAGAACATTTACGCAT
ACTCCAGCGCTCCAGTCGCAGAACTATTATCTGCGTAAAAACG
CAGGCGAAGGATGCTCTTTGACAATTCATTTTGTGCAACTTGT
GGAGCG
(SEQ ID NO: 172)
>3300009632|Ga0116102_1009001_2|P
ACCTGCGCGGCTTGCGGCTCGCATTTCGAAGCGGGGGCCGGGC
TCATGGGGCGCTGCGAGCAGGGGCACGTGATGGACCAGGACTG
GAATGCGGCCGGGAACATCTACGCAGGCCGAGCGGGCGCCACC
GCGCAAGCGGCAGTCGGGTAAGAATGGGGGCGGCACGAGGAGG
CGACCAGGTGCGTTTGCATCCCGCGTGGGGGGTCGGCACATCA
GAGTCCAGCCGTACGCAGGCTGGGGCTAGCAACTGATGCACTG
GGGGGTAAGAACGGGTATTTGCGAAAAGGGTCATGCTACGGGC
CAGGACCTTGACGGAGCAGAGAGCAACTACGCAAACCGGGGCG
GGGCTGCCGCCGCCGCGGGGTTTGCGTAAAAGCTGTGCCGACA
AATGCGCCAAATATATGTTAGTGTTGTGGGTTGTGGAGCGGTT
CGTGCTTCCCTCGCGGCGGGAATCGGTCAGATGCTGCGGGATC
AATTATCCGTTTTCGGATCTGCTTGACGCACGCGCTCCGAAAG
CTCACCATTTACACAAGACCTCGACGGGATCGGTCTGGCAAGC
GACGACCCTGACACCTGCGGAGATGGCGTCGATCAGCTACTCC
GATTCAGAGCAATCCAGTAGCAGAATGGAACGTGTGGTTGGGG
CAGAAGCGCGCAGCTCTCGAACGACCTTAACACCCCCTGGGGA
CCACCTTCCAGATTCGAGCTGATCACAGCTGCATTCGGGCGAT
ACTCGGCTACTTGCTCAAAAACTCCTTTGGTGTGTGTTTAGCC
CCCAACAACCAAGTGCTTGCGCTGCTTTTTGAGCGCACGGCAC
AGCACCTTGCCGACCAGCGCTGTGCGTTGGGCGACGACAACTG
ATATAGGCACGGGCGTGATTCCCTCCTAACCCGAAGTTCCAAG
AGGCTGCAGAGAGTATTCTACTGATTCACTGAGAGATAGTAGC
AAACTCAATTTCCCGTTACTGGGAACGTACGAATCAGTTCGCG
C
(SEQ ID NO: 173)
>3300012971|Ga0126369_10127246_1|M
ACCCAATGCCAGTCCAGCATGTGCAGAGAACACCAATGATTGA
AGAGAAGACTGCTAAGCGTAAAAACCCAGTCAGGCGAGAGCGC
CTGAACAAAGACTGGCCGATCAAGGTCTACACCTATGACTGTT
GGCCGCAAGAAAAGCCGCTCAAGGCGCTATGGGACACGGCGCA
TGAGATGAGGCGGTCTGATGCGTTGGAATATTTGCGGGGATTG
ATTG
(SEQ ID NO: 174)

### Example 5—Functional Validation of Three Engineered CLUST.099129 CRISPR-Cas Systems

Having identified components of CLUST.099129 CRISPR-Cas systems, three loci were selected for functional validation: 1) a locus from the metagenomic source designated SRR6837557 (SEQ ID NO: 101), 2) a locus from the metagenomic source designated 3300012971 (SEQ ID NO: 102), and 3) a locus from the metagenomic source 3300005764 (SEQ ID NO: 103).

### DNA Synthesis and Effector Library Cloning

To test the activity of the exemplary CLUST.099129 CRISPR-Cas systems, systems were designed and synthesized using a pET28a(+) vector. Briefly, an E. coli codon-optimized nucleic acid sequence encoding the CLUST.099129 SRR6837557 effector (SEQ ID NO: 101 shown in TABLE 10), an E. coli codon-optimized nucleic acid sequence encoding CLUST.099129 3300012971 effector (SEQ ID NO: 102 shown in TABLE 10), and an E. coli codon-optimized nucleic acid sequence encoding CLUST.099129 3300005764 effector (SEQ ID NO: 103 shown in TABLE 10) were synthesized (Genscript) and individually cloned into a custom expression system derived from pET-28a(+) (EMD-Millipore). The vectors included the nucleic acid encoding CLUST.099129 effectors under the control of a lac promoter and an E. coli ribosome binding sequence. The vector also included an acceptor site for a CRISPR array library driven by a J23119 promoter following the open reading frame for the CLUST.099129 effector. The non-coding sequence used for the CLUST.099129 SRR6837557 effector (SEQ ID NO: 101) is set forth in SEQ ID NO: 163, the non-coding sequence used for the CLUST.099129 3300012971 effector (SEQ ID NO: 102) is set forth in SEQ ID NO: 174, and the non-coding sequence used for the CLUST.099129 3300005764 effector (SEQ ID NO: 103) is set forth in SEQ ID NO: 170, as shown in TABLE 12. An additional condition was tested, wherein the CLUST.099129 SRR6837557 effector (SEQ ID NO: 101) was individually cloned into pET28a(+) without the non-coding sequence. See FIG. 1A.

An oligonucleotide library synthesis (OLS) pool containing “repeat-spacer-repeat” sequences was computationally designed, where “repeat” represents the consensus direct repeat sequence found in the CRISPR array associated with the effector, and “spacer” represents sequences tiling the pACYC184 plasmid or E. coli essential genes. In particular, the repeat sequence used for the CLUST.099129 SRR6837557 effector (SEQ ID NO: 101) is set forth in SEQ ID NO: 146, the repeat sequence used for the CLUST.099129 3300012971 effector (SEQ ID NO: 102) is set forth in SEQ ID NO: 147, and the repeat sequence used for the CLUST.099129 3300005764 effector (SEQ ID NO: 103) is set forth in SEQ ID NO: 148, as shown in TABLE 11. The spacer length was determined by the mode of the spacer lengths found in the endogenous CRISPR array. The repeat-spacer-repeat sequence was appended with restriction sites enabling the bi-directional cloning of the fragment into the aforementioned CRISPR array library acceptor site, as well as unique PCR priming sites to enable specific amplification of a specific repeat-spacer-repeat library from a larger pool.

Next, the repeat-spacer-repeat library was cloned into the plasmid using the Golden Gate assembly method. Briefly, each repeat-spacer-repeat was first amplified from the OLS pool (Agilent Genomics) using unique PCR primers and pre-linearized the plasmid backbone using BsaI to reduce potential background. Both DNA fragments were purified with Ampure XP (Beckman Coulter) prior to addition to Golden Gate Assembly Master Mix (New England Biolabs) and incubated per the manufacturer's instructions. The Golden Gate reaction was further purified and concentrated to enable maximum transformation efficiency in the subsequent steps of the bacterial screen.

The plasmid library containing the distinct repeat-spacer-repeat elements and CRISPR effectors was electroporated into E. Cloni electrocompetent E. coli (Lucigen) using a Gene Pulser Xcell® (Bio-rad) following the protocol recommended by Lucigen. The library was either co-transformed with purified pACYC184 plasmid or directly transformed into pACYC184-containing E. Cloni electrocompetent E. coli (Lucigen), plated onto agar containing chloramphenicol (Fisher), tetracycline (Alfa Aesar), and kanamycin (Alfa Aesar) in BioAssay® dishes (Thermo Fisher), and incubated for 10-12 hours at 37° C. After estimation of approximate colony count to ensure sufficient library representation on the bacterial plate, the bacteria were harvested, and plasmid DNA WAS extracted using a QIAprep Spin Miniprep® Kit (Qiagen) to create an “output library.” By performing a PCR using custom primers containing barcodes and sites compatible with Illumina sequencing chemistry, a barcoded next generation sequencing library was generated from both the pre-transformation “input library” and the post-harvest “output library,” which were then pooled and loaded onto a Nextseq 550 (Illumina) to evaluate the effectors. At least two independent biological replicates were performed for each screen to ensure consistency. See FIG. 1B.

### Bacterial Screen Sequencing Analysis

Next generation sequencing data for screen input and output libraries were demultiplexed using Illumina bcl2fastq. Reads in resulting fastq files for each sample contained the CRISPR array elements for the screening plasmid library. The direct repeat sequence of the CRISPR array was used to determine the array orientation, and the spacer sequence was mapped to the source (pACYC184 or E. Cloni) or negative control sequence (GFP) to determine the corresponding target. For each sample, the total number of reads for each unique array element (rₐ) in a given plasmid library was counted and normalized as follows: (rₐ+1)/total reads for all library array elements. The depletion score was calculated by dividing normalized output reads for a given array element by normalized input reads.

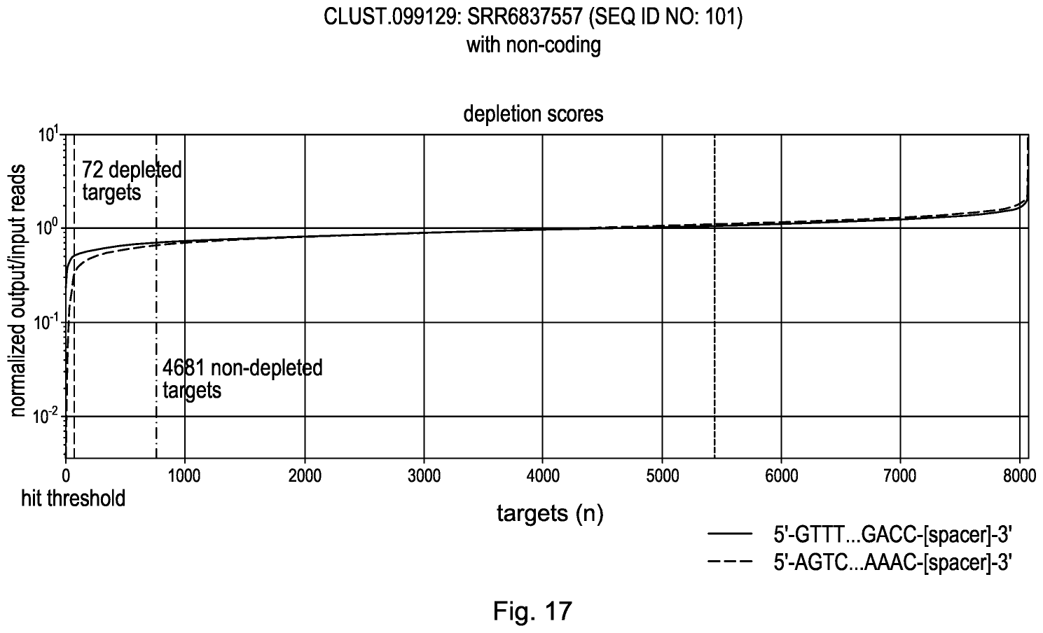

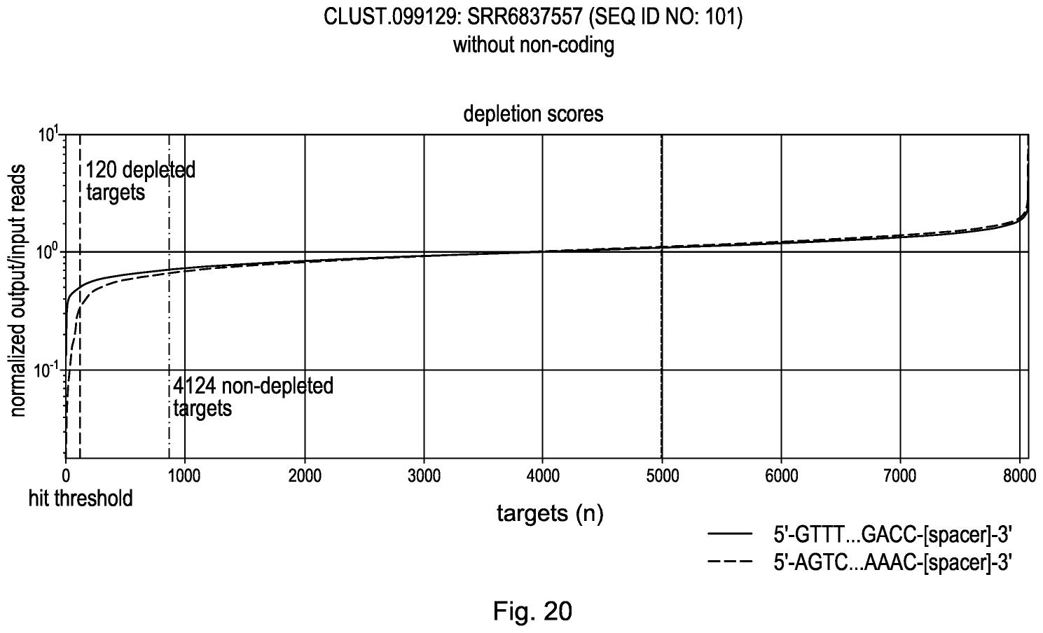

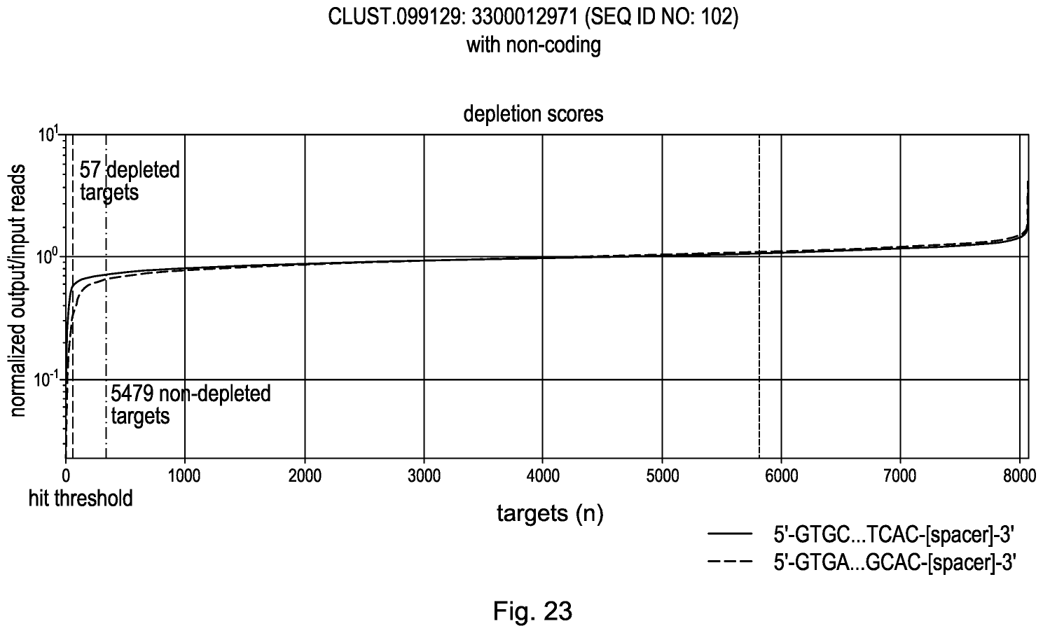

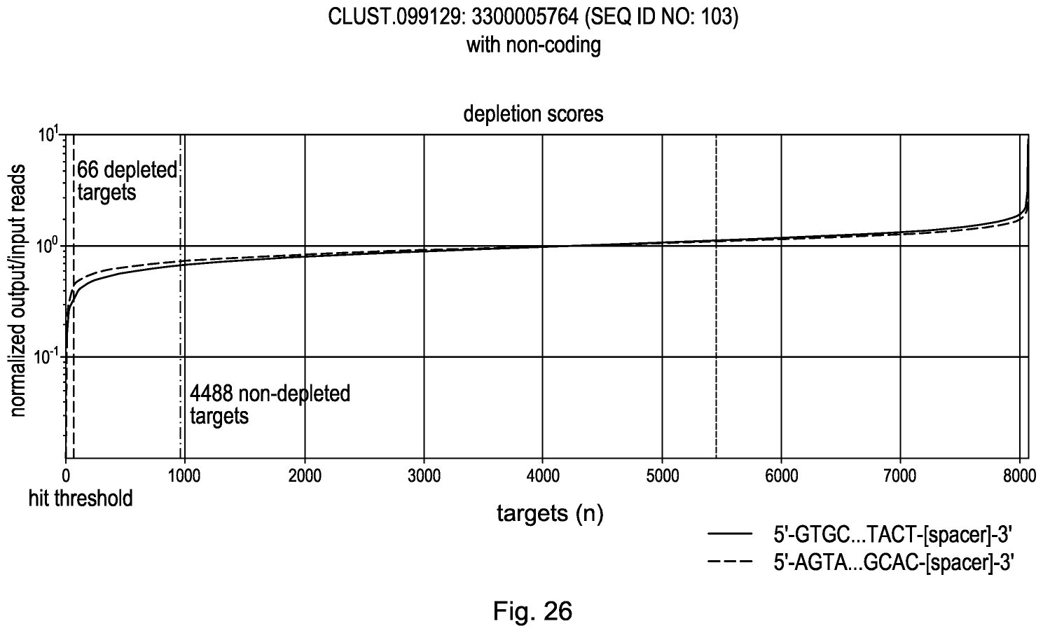

To identify specific parameters resulting in enzymatic activity and bacterial cell death, next generation sequencing (NGS) was used to quantify and compare the representation of individual CRISPR arrays (i.e., repeat-spacer-repeat) in the PCR product of the input and output plasmid libraries. The array depletion ratio was defined as the normalized output read count divided by the normalized input read count. An array was considered to be “strongly depleted” if the depletion ratio was less than 0.3 (more than 3-fold depletion), depicted by the blue dashed line in FIG. 17, FIG. 20, FIG. 23, and FIG. 26. When calculating the array depletion ratio across biological replicates, the maximum depletion ratio value for a given CRISPR array was taken across all experiments (i.e. a strongly depleted array must be strongly depleted in all biological replicates). A matrix including array depletion ratios and the following features were generated for each spacer target: target strand, transcript targeting, ORI targeting, target sequence motifs, flanking sequence motifs, and target secondary structure. The degree to which different features in this matrix explained target depletion for CLUST.099129 systems was investigated.

FIG. 17, FIG. 23, and FIG. 26 show the degree of interference activity of the engineered compositions, with a non-coding sequence, by plotting for a given target the normalized ratio of sequencing reads in the screen output versus the screen input. The results are plotted for each DR transcriptional orientation. In the functional screen for each composition, an active effector complexed with an active RNA guide will interfere with the ability of the pACYC184 to confer E. coli resistance to chloramphenicol and tetracycline, resulting in cell death and depletion of the spacer element within the pool. Comparison of the results of deep sequencing the initial DNA library (screen input) versus the surviving transformed E. coli (screen output) suggests specific target sequences and DR transcriptional orientations that enable an active, programmable CRISPR system. The screen also indicates that the effector complex is only active with one orientation of the DR. As such, the screen indicated that the CLUST.099129 SRR6837557 effector was active in the “reverse” orientation (5′-AGTC . . . AAAC-[spacer]-3′) of the DR (FIG. 17), that the CLUST.099129 3300012971 was active in the reverse orientation (5′-GTGA . . . GCAC-[spacer]-3′) of the DR (FIG. 23), and that the CLUST.099129 3300005764 effector was active in the forward orientation (5′-GTGC . . . TACT-[spacer]-3′) of the DR (FIG. 26).

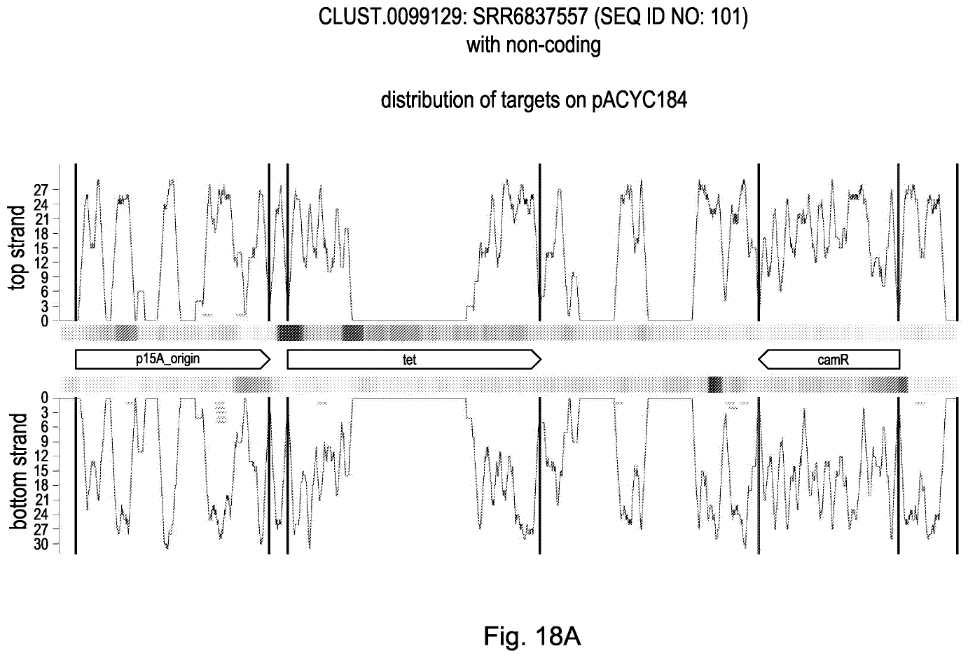

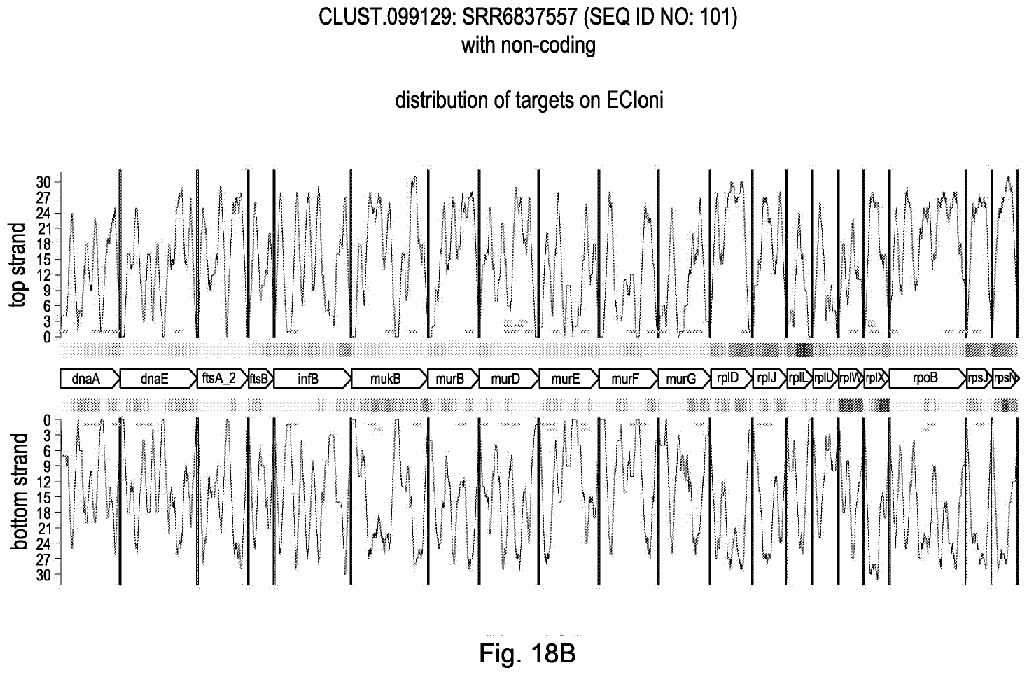

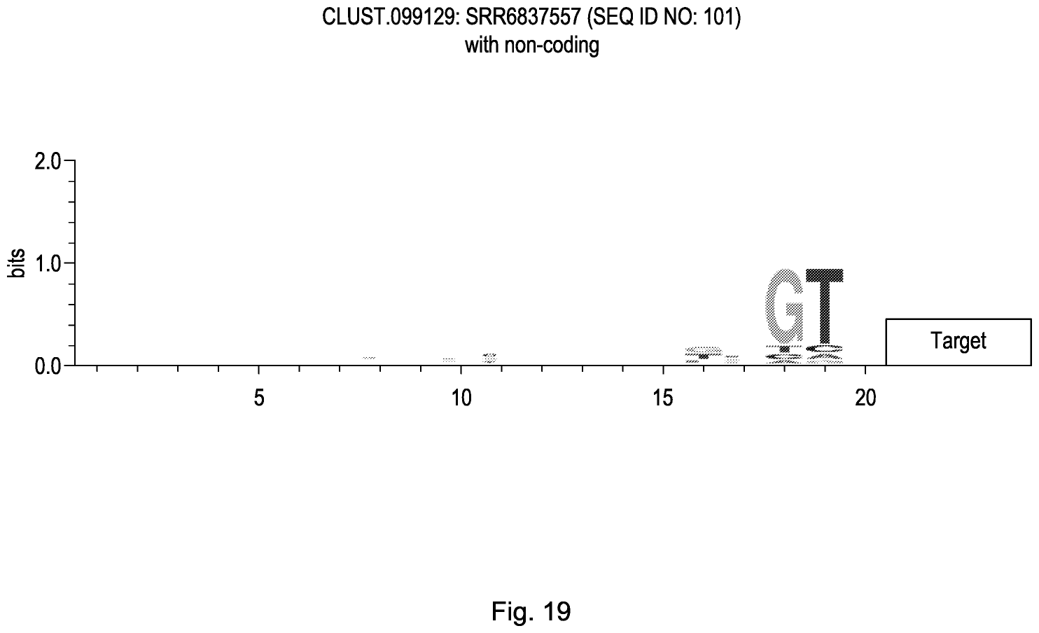

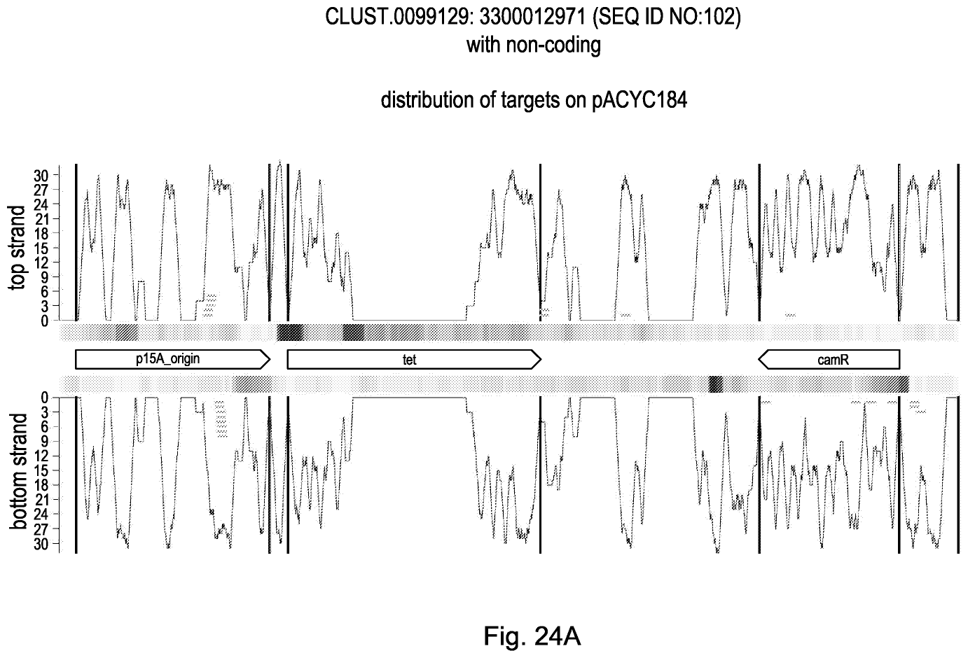

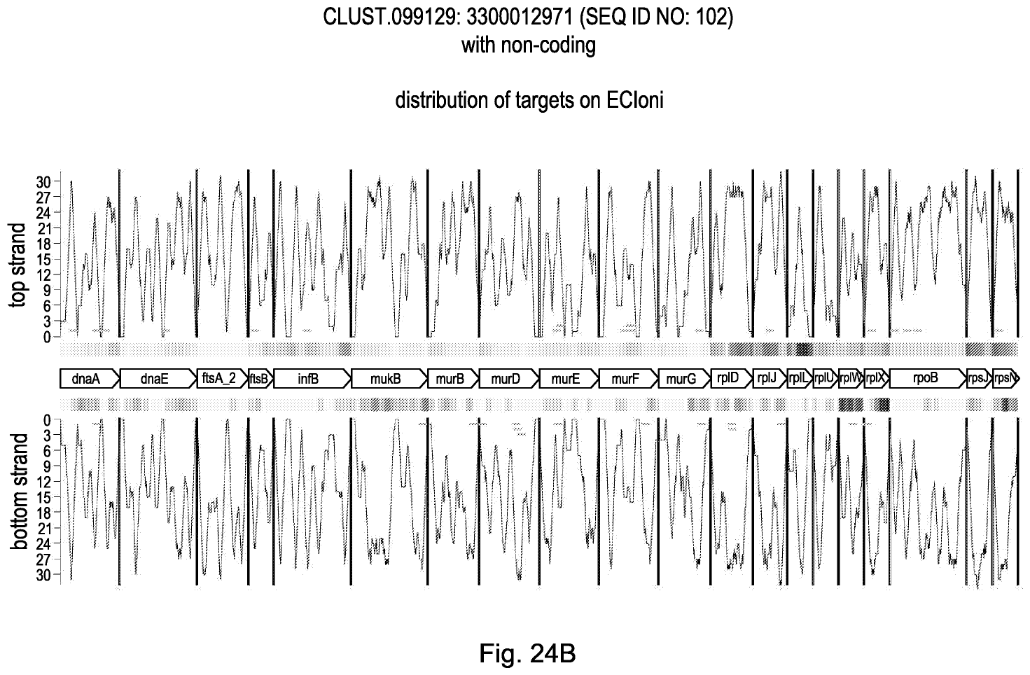

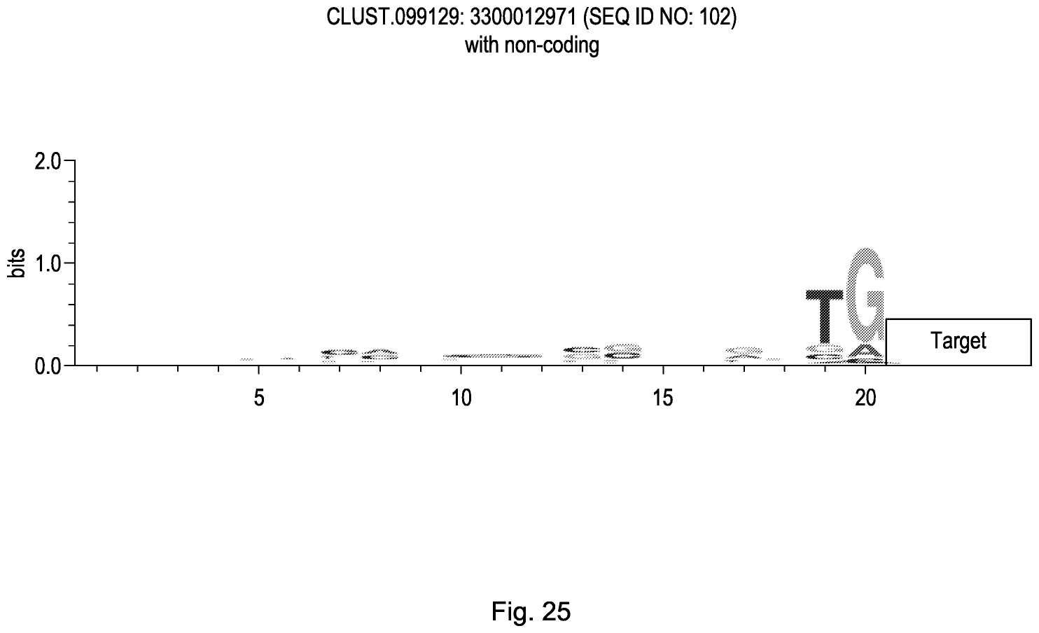

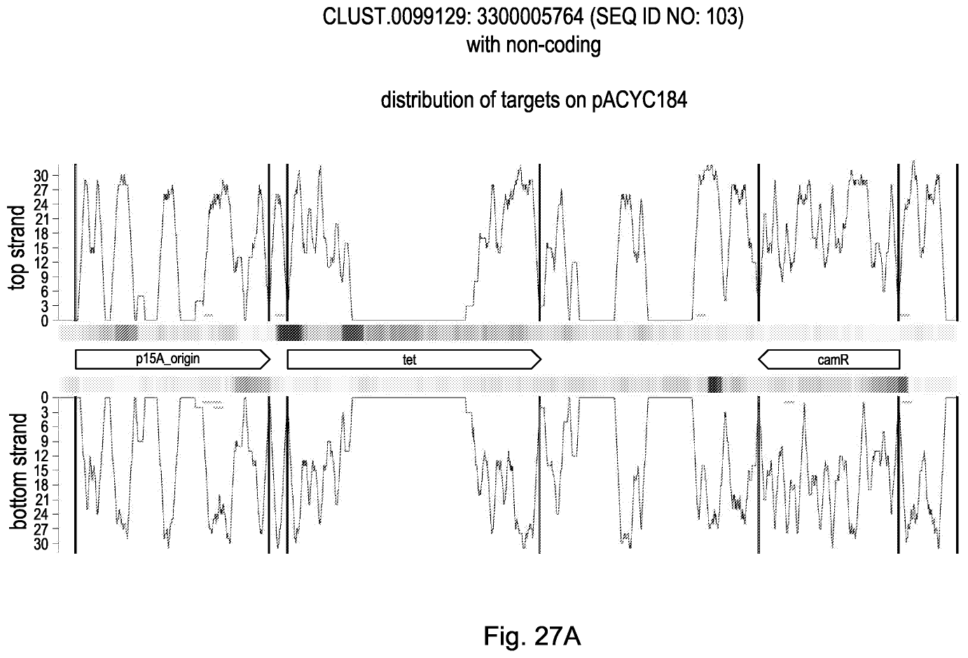

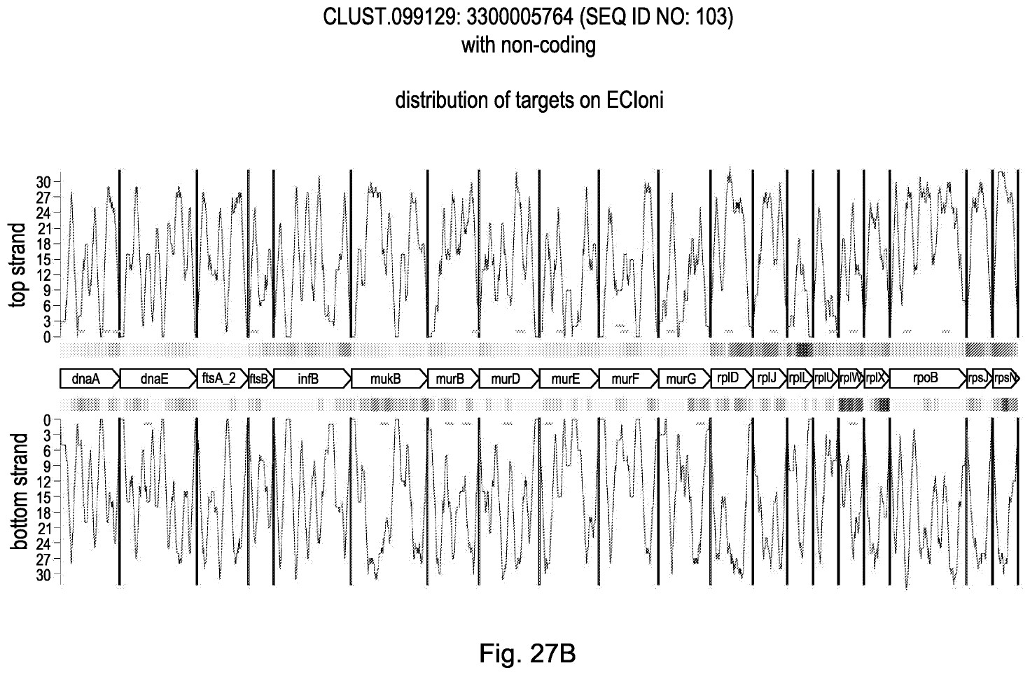

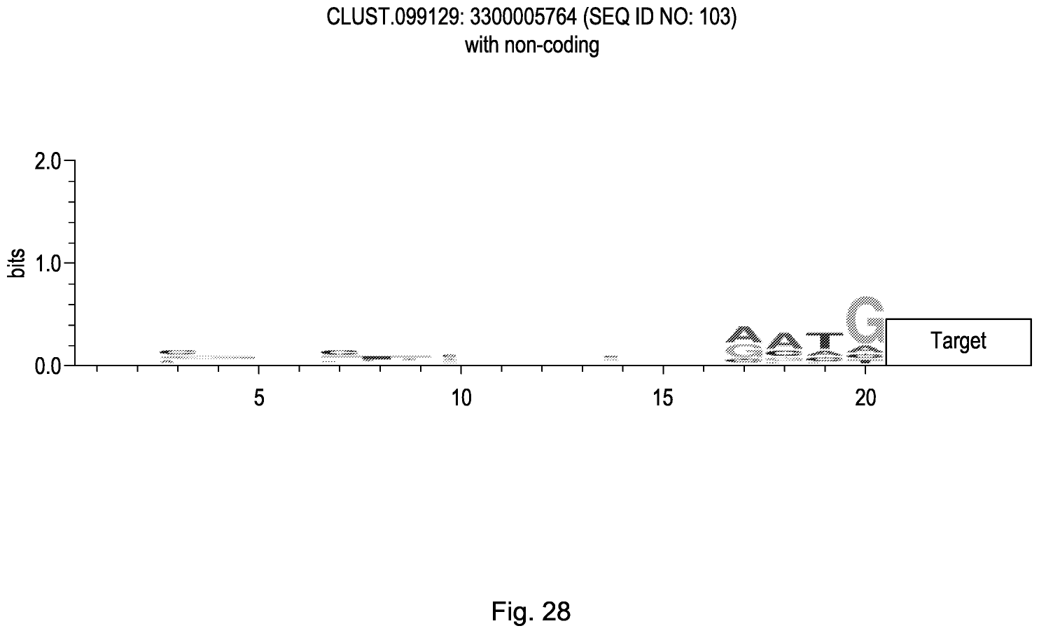

FIG. 18A and FIG. 18B depict the location of strongly depleted targets for the CLUST.099129 SRR6837557 effector (plus non-coding sequence) targeting pACYC184 and E. coli E. Cloni essential genes, respectively FIG. 24A and FIG. 24B depict the location of strongly depleted targets for the CLUST.099129 3300012971 effector (plus non-coding sequence) targeting pACYC184 and E. coli E. Cloni essential genes, respectively. FIG. 27A and FIG. 27B depict the location of strongly depleted targets for the CLUST.099129 3300005764 effector (plus non-coding sequence) targeting pACYC184 and E. coli E. Cloni essential genes, respectively. Flanking sequences of depleted targets were analyzed to determine the PAM for CLUST.099129 effectors. WebLogo representations (Crooks et al., Genome Research 14: 1188-90, 2004) of the PAM sequences for CLUST.099129 SRR6837557, CLUST.099129 3300012971, and CLUST.099129 3300005764 are shown in FIG. 19, FIG. 25, and FIG. 14, respectively. The “20” position corresponds to the nucleotide adjacent to the 5′ end of the target. As such, multiple effectors of CLUST.099129 revealed activity in vivo.

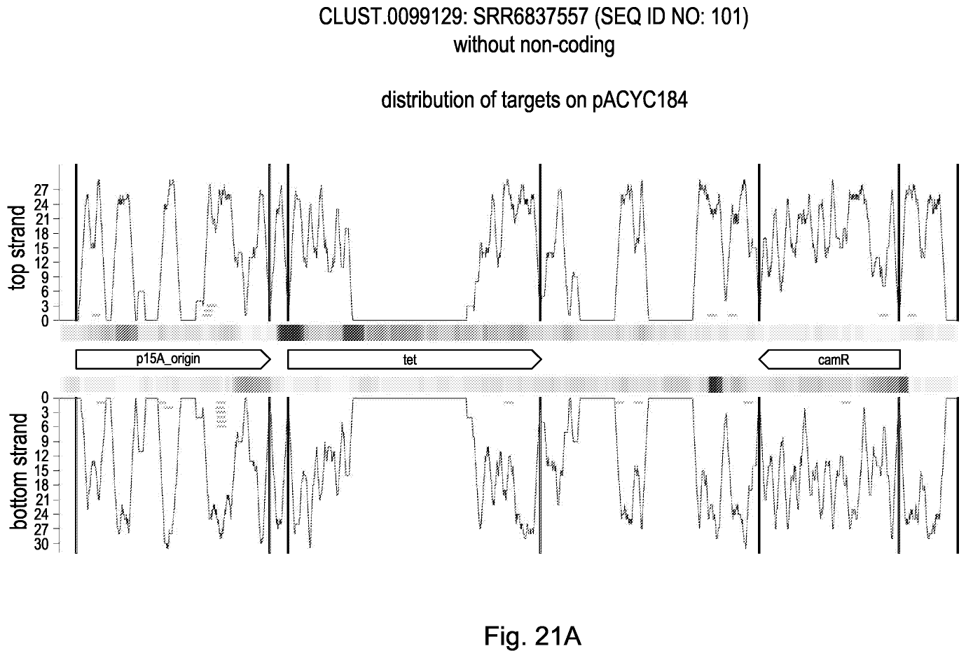

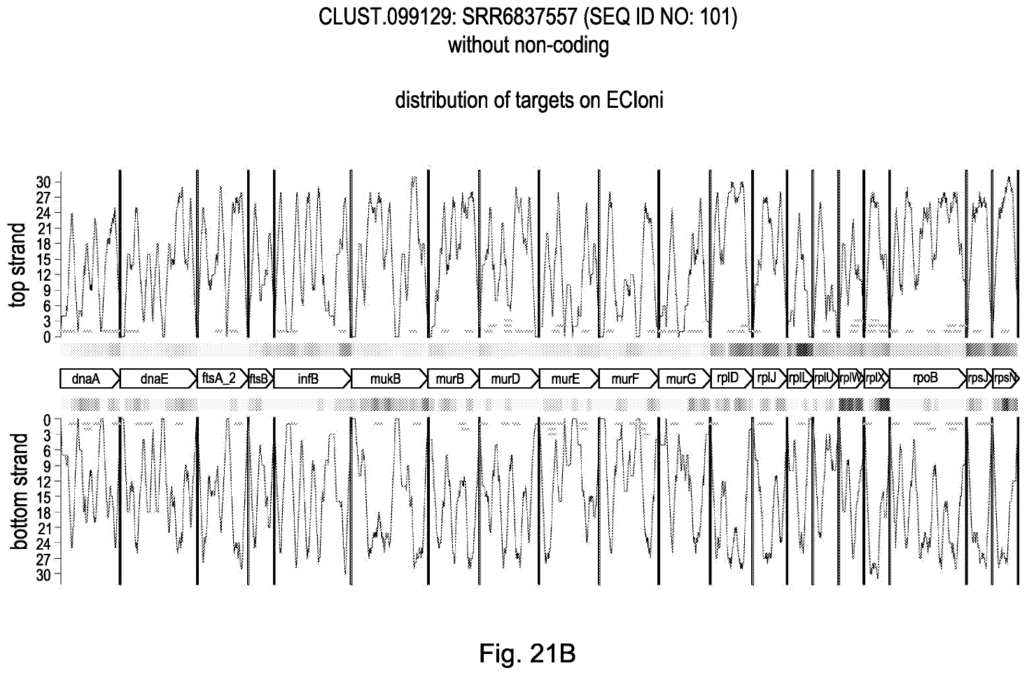

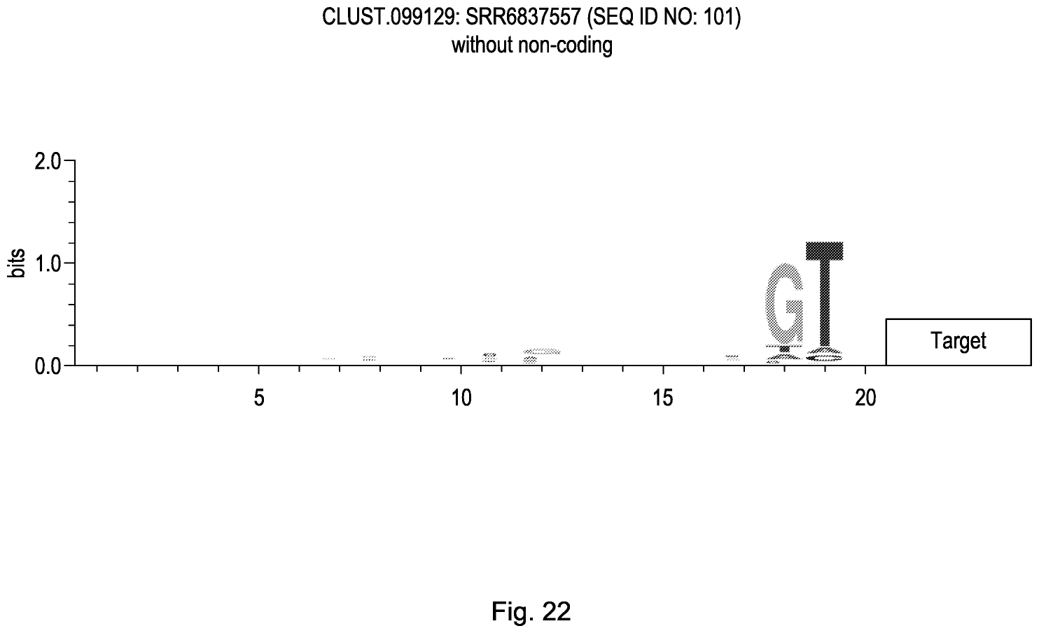

Furthermore, FIG. 20 shows that the CLUST.099129 SRR6837557 effector retains activity in the absence of the non-coding sequence. In agreement with FIG. 17, the CLUST.099129 SRR6837557 effector, without the non-coding sequence, was active in the reverse orientation (5′-AGTC . . . AAAC-[spacer]-3′) of the DR. FIG. 21A and FIG. 21B depict the locations of the strongly depleted targets for the CLUST.099129 SRR6837557 effector, without the non-coding sequence, targeting pACYC184 and E. coli E. Cloni essential genes, respectively. A WebLogo of the PAM sequence for CLUST.099129 SRR6837557, without the non-coding sequence, is shown in FIG. 22, where the “20” position corresponds to the nucleotide adjacent to the 5′ end of the target. This result suggests that effectors of CLUST.099129 do not require a tracrRNA. CLUST.099129 effectors may thus be self-processing, allowing for ease in multiplexing.

### Example 6—Targeting of GFP by a CLUST.099129 Effector

This Example describes use of a fluorescence depletion assay (FDA) to measure activity of a CLUST.099129 effector.

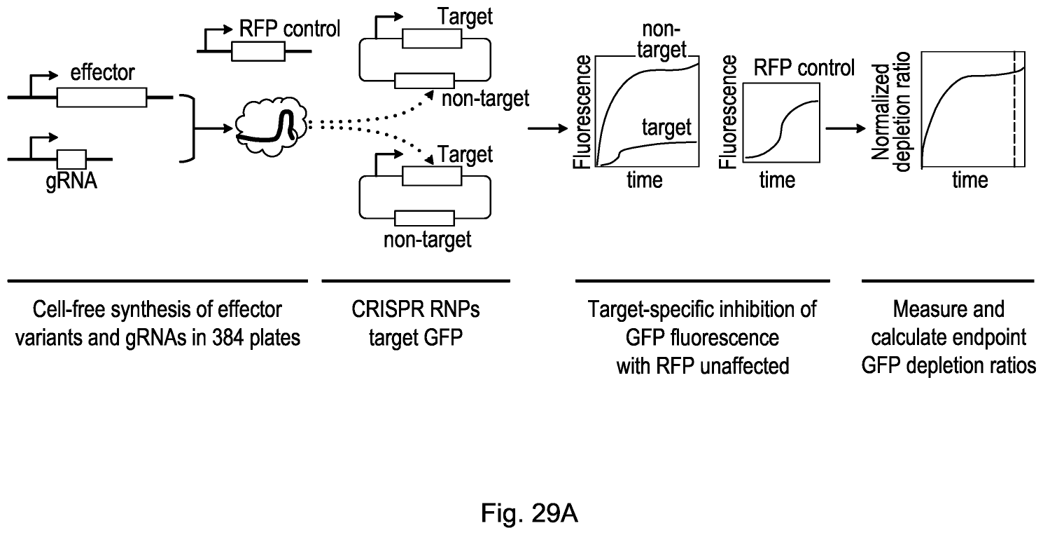

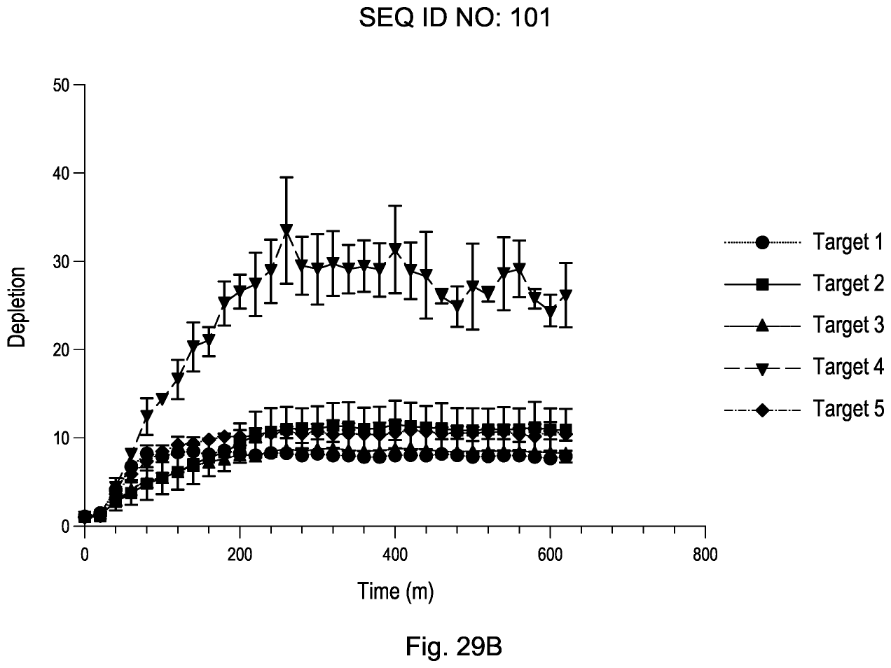

In this assay, an active CRISPR system designed to target GFP binds and cleaves the double-stranded DNA region encoding GFP, resulting in depletion of GFP fluorescence. The FDA assay involves in vitro transcription and translation, allowing production of an RNP from a DNA template encoding a CLUST.099129 effector and a DNA template containing a pre-crRNA sequence under a T7 promoter with direct repeat (DR)-spacer-direct repeat (DR); the spacer targeted GFP. In the same one-pot reaction, GFP and RFP were also produced as both the target and the fluorescence reporter (FIG. 29A). The target GFP plasmid sequence is set forth in SEQ ID NO: 192, and the RFP plasmid sequence is set forth in SEQ ID NO: 193. GFP and RFP fluorescence values were measured every 20 min at 37° C. for 12 hr, using a TECAN Infinite F Plex plate reader. Since RFP was not targeted, its fluorescence was not affected and was therefore used as an internal signal control.

SEQ ID NO: 192
ccccttgtattactgtttatgtaagcagacaggatgcgtc
cggcgtagaggatcgagatctcCAAAAAATGGCTGTTTTT
GAAAAAAATTCTAAAGGTTGTTTTACGACAGACGATAACA
GGGTTgaaataattttgtttaactttaagaaggagATTTA
AATatgAAAATCGAAGAAGGTAAAGGTCACCATCACCATC
ACCACggatccatgacggcattgacggaaggtgcaaaact
gtttgagaaagagatcccgtatatcaccgaactggaaggc
gacgtcgaaggtatgaaatttatcattaaaggcgagggta
ccggtgacgcgaccacgggtaccattaaagcgaaatacat
ctgcactacgggcgacctgccggtcccgtgggcaaccctg
gtgagcaccctgagctacggtgttcagtgtttcgccaagt
acccgagccacatcaaggatttctttaagagcgccatgcc
ggaaggttatacccaagagcgtaccatcagcttcgaaggc
gacggcgtgtacaagacgcgtgctatggttacctacgaac
gcggttctatctacaatcgtgtcacgctgactggtgagaa
ctttaagaaagacggtcacattctgcgtaagaacgttgca
ttccaatgcccgccaagcattctgtatattctgcctgaca
ccgttaacaatggcatccgcgttgagttcaaccaggcgta
cgatattgaaggtgtgaccgaaaaactggttaccaaatgc
agccaaatgaatcgtccgttggcgggctccgcggcagtgc
atatcccgcgttatcatcacattacctaccacaccaaact
gagcaaagaccgcgacgagcgccgtgatcacatgtgtctg
gtagaggtcgtgaaagcggttgatctggacacgtatcagT
AATAAaaagcccgaaaggaagctgagttggctgctgccac
cgctgagcaataactagcataaccccttggggcctctaaa
cgggtcttgaggggttttttgctgaaaggaggaactatat
ccggCTTCCTCGCTCACTGACTCGCTGCGCTCGGTCGTTC
GGCTGCGGCGAGCGGTATCAGCTCACTCAAAGGCGGTAAT
ACGGTTATCCACAGAATCAGGGGATAACGCAGGAAAGAAC
ATGTGAGCAAAAGGCCAGCAAAAGGCCAGGAACCGTAAAA
AGGCCGCGTTGCTGGCGTTTTTCCATAGGCTCCGCCCCCC
TGACGAGCATCACAAAAATCGACGCTCAAGTCAGAGGTGG
CGAAACCCGACAGGACTATAAAGATACCAGGCGTTTCCCC
CTGGAAGCTCCCTCGTGCGCTCTCCTGTTCCGACCCTGCC
GCTTACCGGATACCTGTCCGCCTTTCTCCCTTCGGGAAGC
GTGGCGCTTTCTCATAGCTCACGCTGTAGGTATCTCAGTT
CGGTGTAGGTCGTTCGCTCCAAGCTGGGCTGTGTGCACGA
ACCCCCCGTTCAGCCCGACCGCTGCGCCTTATCCGGTAAC
TATCGTCTTGAGTCCAACCCGGTAAGACACGACTTATCGC
CACTGGCAGCAGCCACTGGTAACAGGATTAGCAGAGCGAG
GTATGTAGGCGGTGCTACAGAGTTCTTGAAGTGGTGGCCT
AACTACGGCTACACTAGAAGAACAGTATTTGGTATCTGCG
CTCTGCTGAAGCCAGTTACCTTCGGAAAAAGAGTTGGTAG
CTCTTGATCCGGCAAACAAACCACCGCTGGTAGCGGTGGT
TTTTTTGTTTGCAAGCAGCAGATTACGCGCAGAAAAAAAG
GATCTCAAGAAGATCCTTTGATCTTTTCTACGGGGTCTGA
CGCTCAGTGGAACGAAAACTCACGggtggcacttttcggg
gaaatgtgcgcggaacccctatttgtttatttttctaaat
acattcaaatatgtatccgctcatgaattaattcttagaa
aaactcatcgagcatcaaatgaaactgcaatttattcata
tcaggattatcaataccatatttttgaaaaagccgtttct
gtaatgaaggagaaaactcaccgaggcagttccataggat
ggcaagatcctggtatcggtctgcgattccgactcgtcca
acatcaatacaacctattaatttcccctcgtcaaaaataa
ggttatcaagtgagaaatcaccatgagtgacgactgaatc
cggtgagaatggcaaaagtttatgcatttctttccagact
tgttcaacaggccagccattacgctcgtcatcaaaatcac
tcgcatcaaccaaaccgttattcattcgtgattgcgcctg
agcgagacgaaatacgcgatcgctgttaaaaggacaatta
caaacaggaatcgaatgcaaccggcgcaggaacactgcca
gcgcatcaacaatattttcacctgaatcaggatattcttc
taatacctggaatgctgttttcccggggatcgcagtggtg
agtaaccatgcatcatcaggagtacggataaaatgcttga
tggtcggaagaggcataaattccgtcagccagtttagtct
gaccatctcatctgtaacatcattggcaacgctacctttg
ccatgtttcagaaacaactctggcgcatcgggcttcccat
acaatcgatagattgtcgcacctgattgcccgacattatc
gcgagcccatttatacccatataaatcagcatccatgttg
gaatttaatcgcggcctagagcaagacgtttcccgttgaa
tatggctcataaca
SEQ ID NO: 193
ccccttgtattactgtttatgtaagcagacaggatgcgtc
cggcgtagaggatcgagatctcCAAAAAATGGCTGTTTTT
GAAAAAAATTCTAAAGGTTGTTTTACGACAGACGATAACA
GGGTTgaaataattttgtttaactttaagaaggagATTTA
AATatgAAAATCGAAGAAGGTAAAGGTCACCATCACCATC
ACCACggatccaTGGTCAGCAAGGGGGAGGAAGACAATAT
GGCTATTATCAAGGAATTCATGCGCTTCAAGGTGCATATG
GAAGGAAGCGTGAATGGACACGAATTCGAGATCGAAGGCG
AGGGGGAGGGTCGCCCTTATGAAGGCACACAAACAGCTAA
ACTGAAAGTGACGAAGGGAGGGCCGCTTCCCTTCGCTTGG
GACATTCTTTCACCCCAGTTCATGTATGGTTCAAAGGCTT
ATGTCAAGCACCCGGCGGACATTCCAGACTACTTAAAATT
GTCGTTCCCCGAGGGGTTTAAATGGGAACGCGTTATGAAT
TTCGAGGATGGGGGAGTCGTAACGGTTACCCAGGACAGTA
GCCTGCAGGATGGCGAGTTCATCTACAAAGTGAAATTGCG
CGGGACGAACTTCCCTAGCGATGGGCCAGTCATGCAGAAG
AAAACGATGGGATGGGAAGCGTCATCCGAGCGCATGTATC
CTGAAGATGGTGCTTTAAAAGGTGAGATCAAGCAGCGTTT
GAAACTGAAGGACGGGGGCCATTATGATGCTGAAGTTAAA
ACGACATATAAGGCCAAGAAGCCAGTTCAACTGCCAGGGG
CTTATAATGTTAATATTAAATTAGACATTACGAGCCATAA
TGAAGATTACACGATTGTCGAGCAATACGAGCGCGCAGAA
GGACGCCACTCAACGGGGGGCATGGACGAGCTGTACAAGT
AAaaagcccgaaaggaagctgagttggctgctgccaccgc
tgagcaataactagcataaccccttggggcctctaaacgg
gtcttgaggggttttttgctgaaaggaggaactatatccg
gCTTCCTCGCTCACTGACTCGCTGCGCTCGGTCGTTCGGC
TGCGGCGAGCGGTATCAGCTCACTCAAAGGCGGTAATACG
GTTATCCACAGAATCAGGGGATAACGCAGGAAAGAACATG
TGAGCAAAAGGCCAGCAAAAGGCCAGGAACCGTAAAAAGG
CCGCGTTGCTGGCGTTTTTCCATAGGCTCCGCCCCCCTGA
CGAGCATCACAAAAATCGACGCTCAAGTCAGAGGTGGCGA
AACCCGACAGGACTATAAAGATACCAGGCGTTTCCCCCTG
GAAGCTCCCTCGTGCGCTCTCCTGTTCCGACCCTGCCGCT
TACCGGATACCTGTCCGCCTTTCTCCCTTCGGGAAGCGTG
GCGCTTTCTCATAGCTCACGCTGTAGGTATCTCAGTTCGG
TGTAGGTCGTTCGCTCCAAGCTGGGCTGTGTGCACGAACC
CCCCGTTCAGCCCGACCGCTGCGCCTTATCCGGTAACTAT
CGTCTTGAGTCCAACCCGGTAAGACACGACTTATCGCCAC
TGGCAGCAGCCACTGGTAACAGGATTAGCAGAGCGAGGTA
TGTAGGCGGTGCTACAGAGTTCTTGAAGTGGTGGCCTAAC
TACGGCTACACTAGAAGAACAGTATTTGGTATCTGCGCTC
TGCTGAAGCCAGTTACCTTCGGAAAAAGAGTTGGTAGCTC
TTGATCCGGCAAACAAACCACCGCTGGTAGCGGTGGTTTT
TTTGTTTGCAAGCAGCAGATTACGCGCAGAAAAAAAGGAT
CTCAAGAAGATCCTTTGATCTTTTCTACGGGGTCTGACGC
TCAGTGGAACGAAAACTCACGggtggcacttttcggggaa
atgtgcgcggaacccctatttgtttatttttctaaataca
ttcaaatatgtatccgctcatgaattaattcttagaaaaa
ctcatcgagcatcaaatgaaactgcaatttattcatatca
ggattatcaataccatatttttgaaaaagccgtttctgta
atgaaggagaaaactcaccgaggcagttccataggatggc
aagatcctggtatcggtctgcgattccgactcgtccaaca
tcaatacaacctattaatttcccctcgtcaaaaataaggt
tatcaagtgagaaatcaccatgagtgacgactgaatccgg
tgagaatggcaaaagtttatgcatttctttccagacttgt
tcaacaggccagccattacgctcgtcatcaaaatcactcg
catcaaccaaaccgttattcattcgtgattgcgcctgagc
gagacgaaatacgcgatcgctgttaaaaggacaattacaa
acaggaatcgaatgcaaccggcgcaggaacactgccagcg
catcaacaatattttcacctgaatcaggatattcttctaa
tacctggaatgctgttttcccggggatcgcagtggtgagt
aaccatgcatcatcaggagtacggataaaatgcttgatgg
tcggaagaggcataaattccgtcagccagtttagtctgac
catctcatctgtaacatcattggcaacgctacctttgcca
tgtttcagaaacaactctggcgcatcgggcttcccataca
atcgatagattgtcgcacctgattgcccgacattatcgcg
agcccatttatacccatata aatcagcatccatgttgga
atttaatcgcggcctagagcaagacgtttcccgttgaata
tggctcataaca

5 GFP targets (plus 1 non-target) were designed for the effector of SEQ ID NO: 101. RNA guide sequences, target sequences, and the non-target control sequences used for the FDA assay are listed in Table 13. A 5′-GTN-3′ PAM was used for the target sequences.

TABLE 13
RNA guide and Target Sequences for FDA Assay.

| Target | Mature crRNA Sequence | Target Sequence |
|---|---|---|
| Target 1 | ATAACTGTTCAGCGGAGAAGCCGCTGAAACa | aaacaacctttagaatttttttca |
| | aacaacctttagaatttttttca (SEQ ID | (SEQ ID NO: 175) |
| | NO: 194) | |
| Target 2 | ATAACTGTTCAGCGGAGAAGCCGCTGAAACg | gaactcaacgcggatgccattgtt |
| | aactcaacgcggatgccattgtt (SEQ ID | (SEQ ID NO: 176) |
| | NO: 195) | |
| Target 3 | ATAACTGTTCAGCGGAGAAGCCGCTGAAACg | gctcgggtacttggcgaaacactg |
| | ctcgggtacttggcgaaacactg (SEQ ID | (SEQ ID NO: 177) |
| | NO: 196) | |
| Target 4 | ATAACTGTTCAGCGGAGAAGCCGCTGAAACt | tacaagacgcgtgctatggttacc |
| | acaagacgcgtgctatggttacc (SEQ ID | (SEQ ID NO: 178) |
| | NO: 197) | |
| Target 5 | ATAACTGTTCAGCGGAGAAGCCGCTGAAACa | aacaatggcatccgcgttgagttc |
| | acaatggcatccgcgttgagttc (SEQ ID | (SEQ ID NO: 179) |
| | NO: 198) | |
| Non-Target 4 | ATAACTGTTCAGCGGAGAAGCCGCTGAAACa | |
| | ggtgctacatttgaagagataaa (SEQ ID | |
| | NO: 199) | |

GFP signal was normalized to RFP signal, then the average fluorescence of three technical replicates was taken at each time point. GFP fluorescence depletion was then calculated by dividing the GFP signal of an effector incubated with a non-GFP targeting RNA guide (which instead targets a kanamycin resistance gene and does not deplete GFP signal) by the GFP signal of an effector incubated with a GFP targeting RNA guide. The resulting value is referred to as “Depletion” in FIG. 29B.

A Depletion of one or approximately one indicated that there was little to no difference in GFP depletion with respect to a non-GFP targeting pre-crRNA and a GFP targeting pre-crRNA (e.g., 10 RFU/10 RFU=1). A Depletion of greater than one indicated that there was a difference in GFP depletion with respect to a non-GFP targeting pre-crRNA and a GFP targeting pre-crRNA (e.g., 10 RFU/5 RFU=2). Depletion of the GFP signal indicated that the effector formed a functional RNP and interfered with the production of GFP by introducing double-stranded DNA cleavage within the GFP coding region. The extent of the GFP depletion was largely correlated to the specific activity of a CLUST.099129 effector.

FIG. 29B shows depletion curves for RNPs formed by the effector of SEQ ID NO: 101, measured every 20 minutes for each of the GFP targets (Targets 1-5). At each target, the depletion values for RNPs formed with the effector of SEQ ID NO: 101 were greater than one.

This indicated that the CLUST.099129 effector formed a functional RNP capable of interfering with the production of GFP.

### Example 7—Identification of Components of CLUST.342201 CRISPR-Cas Systems

This protein family was identified using the computational methods described above. The CLUST.342201 system comprises single effectors associated with CRISPR systems found in uncultured metagenomic sequences collected from wastewater, freshwater, marine, lake sediment, gut, microbial mat, and soil environments (TABLE 16). Exemplary CLUST.342201 effectors include those shown in TABLES 16 and 17, below. Examples of direct repeat sequences and spacer lengths for these systems are shown in TABLE 18. Optionally, the system includes a tracrRNA that is contained within a non-coding sequence listed in TABLE 19.

TABLE 16
Representative CLUST.342201 Effector Proteins

| | | # | effector | SEQ |
|---|---|---|---|---|
| source | effector accession | spacers | size | ID NO |
| wastewater | 3300006417\|Ga0069787_10357475_9\|M | 3 | 387 | 301 |
| metagenome | | | | |
| aquatic-freshwater | 3300022602\|Ga0248169_134333_3\|P | 5 | 345 | 302 |
| aquatic-freshwater | 3300022602\|Ga0248169_134333_4\|M | 5 | 369 | 303 |
| aquatic-freshwater- | 3300025018\|Ga0210043_1027795_3\|M | 2 | 381 | 304 |
| aquifer | | | | |
| aquatic-freshwater- | 3300020198\|Ga0194120_10001032_39\|P | 4 | 391 | 305 |
| freshwater lake | | | | |
| aquatic-freshwater- | 3300014060\|Ga0119967_10019787_9\|M | 5 | 381 | 306 |
| freshwater metagenome | | | | |
| aquatic-freshwater- | 3300020180\|Ga0163155_10006838_6\|M | 2 | 398 | 307 |
| freshwater microbial | | | | |
| mat | | | | |
| aquatic-freshwater- | 3300020180\|Ga0163155_10006838_8\|P | 2 | 419 | 308 |
| freshwater microbial | | | | |
| mat | | | | |
| aquatic-freshwater- | 3300020219\|Ga0163146_10008638_12\|M | 2 | 332 | 309 |
| freshwater microbial | | | | |
| mat | | | | |
| aquatic-freshwater- | 3300020219\|Ga0163146_10008638_11\|P | 2 | 398 | 310 |
| freshwater microbial | | | | |
| mat | | | | |
| aquatic-freshwater- | 3300001380\|JGI1356J14229_10025169_2\|P | 2 | 297 | 311 |
| groundwater | | | | |
| aquatic-freshwater- | 3300014613\|Ga0180008_1001166_14\|P | 2 | 382 | 312 |
| groundwater | | | | |
| aquatic-freshwater- | 3300014613\|Ga0180008_1001166_12\|M | 2 | 374 | 313 |
| groundwater | | | | |
| aquatic-freshwater- | 3300014656\|Ga0180007_10002258_14\|P | 2 | 382 | 314 |
| groundwater | | | | |
| aquatic-freshwater- | 3300014656\|Ga0180007_10002258_12\|M | 2 | 374 | 315 |
| groundwater | | | | |
| aquatic-freshwater- | 3300013127\|Ga0172365_10076875_1\|P | 7 | 398 | 316 |
| sediment | | | | |
| aquatic-marine-aqueous | 3300007960\|Ga0099850_1017825_5\|M | 4 | 365 | 317 |
| aquatic-marine-aqueous | 3300025687\|Ga0208019_1031224_1\|P | 3 | 389 | 318 |
| aquatic-marine-aqueous | 3300025687\|Ga0208019_1031224_1\|M | 3 | 365 | 319 |
| aquatic-non marine | 3300017963\|Ga0180437_10001136_84\|M | 3 | 351 | 320 |
| saline and alkaline - | | | | |
| hypersaline lake | | | | |
| sediment | | | | |
| aquatic-non marine | 3300017963\|Ga0180437_10001136_87\|P | 3 | 408 | 321 |
| saline and alkaline - | | | | |
| hypersaline lake | | | | |
| sediment | | | | |
| aquatic-non marine | 3300017971\|Ga0180438_10021976_13\|P | 2 | 408 | 322 |
| saline and alkaline - | | | | |
| hypersaline lake | | | | |
| sediment | | | | |
| aquatic-non marine | 3300017971\|Ga0180438_10021976_11\|M | 2 | 360 | 323 |
| saline and alkaline - | | | | |
| hypersaline lake | | | | |
| sediment | | | | |
| aquatic-non marine | 3300025586\|Ga0207996_1000986_11\|P | 4 | 399 | 324 |
| saline and alkaline- | | | | |
| saline lake | | | | |
| aquatic-non marine | 3300025642\|Ga0208648_1003027_2\|M | 2 | 399 | 325 |
| saline and alkaline- | | | | |
| saline lake | | | | |
| aquatic-non marine | 3300028299\|Ga0268276_1017374_5\|P | 2 | 403 | 326 |
| saline and alkaline- | | | | |
| saline water | | | | |
| aquatic-non marine | 3300028299\|Ga0268276_1017374_4\|M | 2 | 384 | 327 |
| saline and alkaline- | | | | |
| saline water | | | | |
| gut metagenome | SRR1221442_902393_36\|P | 3 | 303 | 328 |
| mammals-digestive | 3300001598\|EMG_10002624_36\|P | 3 | 303 | 329 |
| system-asian elephant | | | | |
| fecal-elephas maximus | | | | |
| microbial mat | ORBV01001191_65\|M | 2 | 332 | 330 |
| metagenome | | | | |
| microbial mat | ORBV01001191_63\|P | 2 | 398 | 331 |
| metagenome | | | | |
| seawater metagenome | OVSM01005865_8\|M | 2 | 331 | 332 |
| seawater metagenome | OVXX01005191_2\|P | 2 | 331 | 333 |
| terrestrial-soil-corn, | 3300005549\|Ga0070704_100000680_37\|P | 3 | 608 | 334 |
| switchgrass and | | | | |
| miscanthus rhizosphere | | | | |
| terrestrial-soil-corn, | 3300005549\|Ga0070704_100000680_28\|M | 3 | 617 | 335 |
| switchgrass and | | | | |
| miscanthus rhizosphere | | | | |
| terrestrial-soil- | 3300010400\|Ga0134122_10000002_228\|P | 5 | 602 | 336 |
| terrestrial soil | | | | |
| terrestrial-soil-vadose | 3300009089\|Ga0099828_10012481_2\|M | 4 | 341 | 337 |
| zone soil | | | | |
| terrestrial-soil-vadose | 3300012201\|Ga0137365_10010044_6\|P | 4 | 573 | 338 |
| zone soil | | | | |
| terrestrial-soil-vadose | 3300012356\|Ga0137371_10006599_2\|M | 4 | 573 | 339 |
| zone soil | | | | |
| wastewater metagenome | 3300006417_501592_1\|M | 6 | 387 | 340 |
| wastewater metagenome | SRR6837571_682758_2\|M | 6 | 387 | 341 |

TABLE 171
Amino Acid Sequences of Representative CLUST.342201 Effector Proteins

>3300006417|Ga0069787_10357475_9|M
[wastewater metagenome]
MLKAHVIRLNPTKEQETYFWRCAGVARFTWNWALAELNAAYEKGERPAVGSLKLEFNRLRNEEGFAPFVGEVQSYAYQQAFGDLQK
ALSSYHDFRKRGMLKPPTSWKGRKDHKPFGWPRFKVRNRSTPAFYLANNGGLRMNGHQVTIQRCPGGPVNMAEPLRFTGKVMGGRV
RYRAGHWYLTVQVDVPVEPDPAHTGPAVGLDVGIKVLAFTSDGVIYDNPKALAHYQRKLRLLQRSLSRQTRGGSNYRKTQAKIARL
HDRIANIRKHALHQVSHEITRDYGLIGLEDLNVSGMLKNGKLARSISDVALGELRRQIEYKADWRGSRVMIVSRWFPSSKTCNDCG
YVMADMLLSVRWWQCPACGAEHDRDGNAAVNIRNEAVKMAGAA (SEQ ID NO: 301)
>3300022602|Ga0248169_134333_3|P
[aquatic-freshwater]
MNSQSLLAALLDVDNAFKRFFTQGAGYPKFASKYDPWQSFSCPQHVRLEGGQVHLPKLGLMDLVLHRQIPSDGTIKNCVIKRSPTG
KYQISLLIERNINEVICAPVIKDQSIGIDLGIKTFAVGSDGYSIQNPRFLSRQLPRLKTEQRKFARMQKDSNNRAKQKKVVAKIHE
DVANARHHFIHQESYRLAVKNHATTVMMETLMIKNMVRNHKLARHITDCGWGMFEKALEYKLAERGKNLIKIDTFLPSTKKCRICK
TKNTAMTLKDRVFVCGSCGHIEDRDLHAAQNVRDFGYEQYTRSLGTCETVKRSPVSISVGADEFAKGSKKFGTGRLEEAPAIAALA
A (SEQ ID NO: 302)
>3300022602|Ga0248169_134333_4|M
[aquatic-freshwater]
MSRRQLQDHLVKKKKKSKFTWLNEVNSQSLLAALLDVDNAFKRFFTQGAGYPKFASKYDPWQSFSCPQHVRLEGGQVHLPKLGLMD
LVLHRQIPSDGTIKNCVIKRSPTGKYQISLLIERNINEVICAPVIKDQSIGIDLGIKTFAVGSDGYSIQNPRFLSRQLPRLKTEQR
KFARMQKDSNNRAKQKKVVAKIHEDVANARHHFIHQESYRLAVKNHATTVMMETLMIKNMVRNHKLARHITDCGWGMFEKALEYKL
AERGKNLIKIDTFLPSTKKCRICKTKNTAMTLKDRVFVCGSCGHIEDRDLHAAQNVRDFGYEQYTRSLGTCETVKRSPVSISVGAD
EFAKGSKKFGTGRLEEAPAIAALAA (SEQ ID NO: 303)
>3300025018|Ga0210043_1027795_3|M
[aquatic-freshwater-aquifer]
MNRAHRIRLNPTAEQAAYFWRCAGVARFTWNWALGEYNAALARGEKPSLTALKLEFNRRRSEDRFAPWVGEVQSYAYQYAFQDLQT
AISRYHERRQADKLKPPAGWKPRKDGKPFGWPRFKARDNTTPAFGLANNGGIRCTGHQAQIQRCPGPVNMAEELRFDGRILSGRVT
YIAGHWYLAVAIELPDPQPEPLPGRVGVDLGIKSLAVTSDGVIYENPKHLRGSQRKLAHLQREFSRRQKGSRNWAKTKAKITRLHA
RIGNQRREVLHEMTTDLVRTYGTVVVEDLNVSGMLQNHHLAQAVSDASFGEIRRQLEYKASAFGSKVVIADRWYPSSKTCNDCGQI
VELTLAERTWTCPRCGVIHDRDLNAARNLRDYSAVTG (SEQ ID NO: 304)
>3300020198|Ga0194120_10001032_39|P
[aquatic-freshwater-freshwater lake]
MRVNKVFRYELKPNEVQKQIMAQHAGSARYAYNWALEKINSKVFNTTNDKKLHALWRQYRPGWWDFSVSKCVPQEALVNLSNSFKN
HRVNPGYFKDPVFKKKFIHDSFRLTGTVKVFANHVQLPILGKIRLKEVIDKYLRSLKSKNTKKLSTKDYKKTDRILSATVTRTADR
WYVSILVDIDIPEITDIPTGVVGIDFGIKKFLTLSNGLKFDHPKALKKHLKLLKRRQKQKDKKQKGSHNRLKSSLKLARLHAKIAN
IRRDYLNKITTWLARSKSVIVLENLNLKGMGKWHALAQALADAGFGLFKSMLQYKCQWYGSKLILADRWYGSSKTCSNCGWYYKDL
SLKERSWICKSCNTDHDRDLNAAINLKKIGVLSHSRGIYACGDSYAG (SEQ ID NO: 305)
>3300014060|Ga0119967_10019787_9|M
[aquatic-freshwater-freshwater metagenome]
MNRAHRIRLNPTAEQAAYFWRCAGVARFTWNWALGEYNAALARGEKPSLTALKLEFNRRRSEDRFAPWVGEVQSYAYQYAFQDLQT
AISRYHERRQADKLKPPAGWKPRKDGKPFGWPRFKARDNTTPAFGLANNGGIRCTGHQAQIQRCPGPVNMAEELRFDGRILSGRVT
YIAGHWYLAVAIELPDPQPEPLPGRVGVDLGIKSLAVTSDGVIYENPKHLRGSQRKLAHLQREFSRRQKGSHNWAKTKAKITRLHA
RIGNQRREVLHEMTTDLVRTYGTVVVEDLNVSGMLQNHHLAQAVSDASFGEIRRQLEYKASAFGSKVVIADRWYPSSKTCNDCGQI
VELTLAERTWTCPRCGVIHDRDLNAARNLRDYSAVTG (SEQ ID NO: 306)
>3300020180|Ga0163155_10006838_6|M
[aquatic-freshwater-freshwater microbial mat]
MATVRYRYRLYPTPAQRVALARTFGAVRFVWNQELARSQIHDAKYQGFSAASRRLTAAKKTTDLAWLNAISCVPIQQSLRNLDVSY
RNFFRGCKSKGPRRGYPRFKSKYDEQSAEYTRSGFKVEHGKLCLAKLGSIEVRWSRLLPSIPKTATVKLDATGRYFVSFTVEVNAQ
PLVGGTPVGIDLGIKCFAALSTGEMVQAPSYKRRERKIAHVQRELARRKNGSRRRAVTKKKLARLHAKISDTRRDFLDKLSTRLVR
SHDVIVVEDLNVSGMVKNHHLARSISRQGWRAFRTMLESKCTRYGRELQVVNRWTPTSQICSTCGHRWGRLDLSVRQVTCEGCGAA
HDRDVNAAKNIVAAGQVETKNGRGGHVRRMVPLGTCAVLDEASTSGPTPESSAF (SEQ ID NO: 307)
>3300020180|Ga0163155_10006838_8|P
[aquatic-freshwater-freshwater microbial mat]
MTTAIERLHGHVLQSMVSCRPMATVRYRYRLYPTPAQRVALARTFGAVRFVWNQELARSQIHDAKYQGFSAASRRLTAAKKTTDLA
WLNAISCVPIQQSLRNLDVSYRNFFRGCKSKGPRRGYPRFKSKYDEQSAEYTRSGFKVEHGKLCLAKLGSIEVRWSRLLPSIPKTA
TVKLDATGRYFVSFTVEVNAQPLVGGTPVGIDLGIKCFAALSTGEMVQAPSYKRRERKIAHVQRELARRKNGSRRRAVTKKKLARL
HAKISDTRRDFLDKLSTRLVRSHDVIVVEDLNVSGMVKNHHLARSISRQGWRAFRTMLESKCTRYGRELQVVNRWTPTSQICSTCG
HRWGRLDLSVRQVTCEGCGAAHDRDVNAAKNIVAAGQVETKNGRGGHVRRMVPLGTCAVLDEASTSGPTPESSAF (SEQ ID
NO: 308)
>3300020219|Ga0163146_10008638_12|M
[aquatic-freshwater-freshwater microbial mat]
MNDVSSVPVQQSIRNLDVAFRRFFNGLKGKGPKVGFPTWKRKDGKQSAEFTRSGFKLQRGKLYIAKVGDVDVRWSRALPSVPKTAT
VTLDAAGRYHVSFTVEKTDVALNGGSPVGVDLGIKTFAALSTGELVHAPDTKRLERRIGKAQRGLSRCRKGSARRGRAKLRVAKLH
AVLADTRRDFLEKFSTRLVREHSLIALEDLNVAGMAKNHCLARAVNRQGWRMFRTMVQSKCERYGREVRIVGRWEPTSQVCSGCGH
RWGRVGLGVRTIACEGCGEVHDRDVNAAKNVVAAGLADTENGRGEWVSRVVSMGTGVPFVEASTAGASPGISAL (SEQ ID NO:
309)
>3300020219|Ga0163146_10008638_11|P
[aquatic-freshwater-freshwater microbial mat]
MASLRYSYRLYPTTEQRRALAQTFGCVRVAWNDALARSQVSGAKYPGFAATSRLLTESKKSPERAWLNDVSSVPVQQSIRNLDVAF
RRFFNGLKGKGPKVGFPTWKRKDGKQSAEFTRSGFKLQRGKLYIAKVGDVDVRWSRALPSVPKTATVTLDAAGRYHVSFTVEKTDV
ALNGGSPVGVDLGIKTFAALSTGELVHAPDTKRLERRIGKAQRGLSRCRKGSARRGRAKLRVAKLHAVLADTRRDFLEKFSTRLVR
EHSLIALEDLNVAGMAKNHCLARAVNRQGWRMFRTMVQSKCERYGREVRIVGRWEPTSQVCSGCGHRWGRVGLGVRTIACEGCGEV
HDRDVNAAKNVVAAGLADTENGRGEWVSRVVSMGTGVPFVEASTAGASPGISAL (SEQ ID NO: 310)
>3300001380|JGI1356J14229_10025169_2|P
[aquatic-freshwater-groundwater]
MKKQGNKAGFPRFKPFEKMKSLHYPQYGNGFFLDKTLEASPFGKIQIVRHREIKGRIKTLSIKREASGKWFACISADAPLIIKFSN
NQPKVGIDLGLETFATLSNGEKVQNPRHIKKYEKRLAFLQRKRSRKTKGSRNRKKANEIAARQYLKLKNTRLDFLHKLSHNLVNSY
SLICLEDLACQEMAENRFGKQINDAGWGRFATMIQYKAESAGAEVIFVNPENTSKTCCICGNLQDMPLNERAYNCKSCGNSLDRDI
NAALNILTRATAGMAGSNASGDETIIPSLKEEAHAFTRG (SEQ ID NO: 311)
>3300014613|Ga0180008_1001166_14|P
[aquatic-freshwater-groundwater]
MHRAHRIRLNPTPEQEKYLWRCAGVARFTWNWALAAYNDTLVRGEKPSIAKLKVEFNRQRAEEAFAPWVGEVQSYAYQYAFQDLRT
AINRYHELRKADKLKPRAGWKPRKDGRPFGWPRFKARALTTPAFGLANNGGVHCAGNLAYLQRCPSPINMAEELRFTGRILSGHVS
YTGGHWHLSVSLDLPDPQPEPLPGRVGVDLGIKSLAVTSDGVVYENPKHLRASQRKLARLQRELARRTKGGKNRAKTQTKIARLHE
RIANQRRETLHEMTTDLVSRYGTVVVEDLNVSGMLSNHRLALAVSDASFGEIRRQLEYKAPAHGSQVLVANQWFPSSKTCNDCGCV
TALTLSDRVWTCPNCGVVHDRDINAAQNLRDYQPVSVA (SEQ ID NO: 312)
>3300014613|Ga0180008_1001166_12|M
[aquatic-freshwater-groundwater]
MNPTPEQEKYLWRCAGVARFTWNWALAAYNDTLVRGEKPSIAKLKVEFNRQRAEEAFAPWVGEVQSYAYQYAFQDLRTAINRYHEL
RKADKLKPRAGWKPRKDGRPFGWPRFKARALTTPAFGLANNGGVHCAGNLAYLQRCPSPINMAEELRFTGRILSGHVSYTGGHWHL
SVSLDLPDPQPEPLPGRVGVDLGIKSLAVTSDGVVYENPKHLRASQRKLARLQRELARRTKGGKNRAKTQTKIARLHERIANQRRE
TLHEMTTDLVSRYGTVVVEDLNVSGMLSNHRLALAVSDASFGEIRRQLEYKAPAHGSQVLVANQWFPSSKTCNDCGCVTALTLSDR
VWTCPNCGVVHDRDINAAQNLRDYQPVSVA (SEQ ID NO: 313)
>3300014656|Ga0180007_10002258_14|P
[aquatic-freshwater-groundwater]
MHRAHRIRLNPTPEQEKYLWRCAGVARFTWNWALAAYNDTLVRGEKPSIAKLKVEFNRQRAEEAFAPWVGEVQSYAYQYAFQDLRT
AINRYHELRKADKLKPRAGWKPRKDGRPFGWPRFKARALTTPAFGLANNGGVHCAGNLAYLQRCPSPINMAEELRFTGRILSGHVS
YTGGHWHLSVSLDLPDPQPEPLPGRVGVDLGIKSLAVTSDGVVYENPKHLRASQRKLARLQRELARRTKGGKNRAKTQTKIARLHE
RIANQRRETLHEMTTDLVSRYGTVVVEDLNVSGMLSNHRLALAVSDASFGEIRRQLEYKAPAHGSQVLVANQWFPSSKTCNDCGCV
TALTLSDRVWTCPNCGVVHDRDINAAQNLRDYQPVSVA (SEQ ID NO: 314)
>3300014656|Ga0180007_10002258_12|M
[aquatic-freshwater-groundwater]
MNPTPEQEKYLWRCAGVARFTWNWALAAYNDTLVRGEKPSIAKLKVEFNRQRAEEAFAPWVGEVQSYAYQYAFQDLRTAINRYHEL
RKADKLKPRAGWKPRKDGRPFGWPRFKARALTTPAFGLANNGGVHCAGNLAYLQRCPSPINMAEELRFTGRILSGHVSYTGGHWHL
SVSLDLPDPQPEPLPGRVGVDLGIKSLAVTSDGVVYENPKHLRASQRKLARLQRELARRTKGGKNRAKTQTKIARLHERIANQRRE
TLHEMTTDLVSRYGTVVVEDLNVSGMLSNHRLALAVSDASFGEIRRQLEYKAPAHGSQVLVANQWFPSSKTCNDCGCVTALTLSDR
VWTCPNCGVVHDRDINAAQNLRDYQPVSVA (SEQ ID NO: 315)
>3300013127|Ga0172365_10076875_1|P
[aquatic-freshwater-sediment]
MTAISISHKIALEPTKLQAAHFALCVGASRKAYNWALDTWNRQWDDGRKPKAADIRKLFNEVKWHDDTLRWMTGARNFVADRSIQN
LGEAWKKHFRAPAQWRKPTFHSTRIHRSFYVSIGNRKAPNIQVQGRRVRLCLEGTNYPEGQARIGWVKMREELRWDGRIESATISR
DEKRWYISFSCSGAENYPHRAGEGLLGVAIGFNTVALSNGTVFDQRRPLERYLVKKQRLSRSLSRKQSRLWNKKTKEVQRQSSANW
EKARARLARLDDRIANLRSDFIHRVTTDIVRTGKALVIEAVNVQRLLKDGRTARAASDVSFFEWKRQLQYKTQVAGGSLQQVPTSI
DLYQVCSRCGRRNYDARKYGKRFVCPHCGLDIENGVNIASNLATVKSAESQACG (SEQ ID NO: 316)
>3300007960|Ga0099850_1017825_5|M
[aquatic-marine-aqueous]
MSRFTWNWALAAYNDGVPFSYLKTEFNRLRSEDGFAPFVSEVQSYAYQQAFNDLNAALSRYFKFQKEGRLKPPAGWKPRKDRKPFG
WPRFKSRDKSTPSFYLANNGGMRFDGHGVVIQKCPGGPVNMAEPLRFDGKIMGGRVSYTGGYWYLAVSVQMGDPEPTADTSAAVGV
DMGIKYRAVTSDGVIYNNPKSLAQNLRKLRKLQRSLSRMRDAAKAEGRKLGESNNYQKRKREIAKLHATIANIRREHAHEMTTELV
REYGVVGVEDLNIRGMVKNRRLSRAVMDASMYQVRTQLDYKLKTSGGELVVVDRWYASSKTCNACGEKVDALPLSIRQWACPSCGA
EHDRDGNAAKNIRDEALRMRG (SEQ ID NO: 317)
>3300025687|Ga0208019_1031224_1|P
[aquatic-marine-aqueous]
MNRAHRIRLNPTTEQEEYFWQCAGVSRFTWNWALAAYNDGVPFSYLKTEFNRLRSEDGFAPFVSEVQSYAYQQAFNDLNAALSRYF
KFQKEGRLKPPAGWKPRKDRKPFGWPRFKSRDKSTPSFYLANNGGMRFDGHGVVIQKCPGGPVNMAEPLRFDGKIMGGRVSYTGGY
WYLAVSVQMDDPEPTADTSAAVGVDMGIKYRAVTSDGVIYNNPKSLAQNLRKLRKLQRSLSRMRDAAKAEGRKLGESNNYQKRKRE
IAKLHATIANIRREHAHEMTTELVREYGVVGVEDLNIRGMVKNRRLSRAVMDASMYQVRTQLDYKLKTSGGELVVVDRWYASSKTC
NACGEKVDALPLSIRQWACPSCGAEHDRDGNAAKNIRDEALRMRG (SEQ ID NO: 318)
>3300025687|Ga0208019_1031224_1|M
[aquatic-marine-aqueous]
MSRFTWNWALAAYNDGVPFSYLKTEFNRLRSEDGFAPFVSEVQSYAYQQAFNDLNAALSRYFKFQKEGRLKPPAGWKPRKDRKPFG
WPRFKSRDKSTPSFYLANNGGMRFDGHGVVIQKCPGGPVNMAEPLRFDGKIMGGRVSYTGGYWYLAVSVQMDDPEPTADTSAAVGV
DMGIKYRAVTSDGVIYNNPKSLAQNLRKLRKLQRSLSRMRDAAKAEGRKLGESNNYQKRKREIAKLHATIANIRREHAHEMTTELV
REYGVVGVEDLNIRGMVKNRRLSRAVMDASMYQVRTQLDYKLKTSGGELVVVDRWYASSKTCNACGEKVDALPLSIRQWACPSCGA
EHDRDGNAAKNIRDEALRMRG (SEQ ID NO: 319)
>3300017963|Ga0180437_10001136_84|M
[aquatic-non marine saline and alkaline-hypersaline lake sediment]
MDETCPELLWVRDELGMNKQIQQVPCEDLEKAWKRFFDWCRARKKGNVGLRRVGPPKFKSSRWMVGMSFGAKQGVEHDPETRKVRI
PSIGWIRHFEKDVDFSNDKIKCCRVVWRASGWYCSLSMDNDIEIKSPLKWKKRKTHSVHLGIRHYAVLSDGNNVDHPRNLERNLDR
LSKLCREWSRKKKGSRRWRIWRDRVRRVHEKIANCRKDFIHKMTDDLLKKYPCLISEKWSISDMVENEKTKRKRRKIKAGRRKPGS
RMMEKLLHRGIMDSSWYEVRRQLKYKSEWRRKPYHETHMGFKSTQTCFHCKHVNDFVTLNMKTFTCRGCGRKIERESNAAANVREM
GFSELGL (SEQ ID NO: 320)
>3300017963|Ga0180437_10001136_87|P
[aquatic-non marine saline and alkaline-hypersaline lake sediment]
MRRSHALKLDPNAGQARKLRLYCIAHCKIWNHFLEKCKKGLIPKKSNAMTHQLGNKRMDETCPELLWVRDELGMNKQIQQVPCEDL
EKAWKRFFDWCRARKKGNVGLRRVGPPKFKSSRWMVGMSFGAKQGVEHDPETRKVRIPSIGWIRHFEKDVDFSNDKIKCCRVVWRA
SGWYCSLSMDNDIEIKSPLKWKKRKTHSVHLGIRHYAVLSDGNNVDHPRNLERNLDRLSKLCREWSRKKKGSRRWRIWRDRVRRVH
EKIANCRKDFIHKMTDDLLKKYPCLISEKWSISDMVENEKTKRKRRKIKAGRRKPGSRMMEKLLHRGIMDSSWYEVRRQLKYKSEW
RRKPYHETHMGFKSTQTCFHCKHVNDFVTLNMKTFTCRGCGRKIERESNAAANVREMGFSELGL (SEQ ID NO: 321)
>3300017971|Ga0180438_10021976_13|P
[aquatic-non marine saline and alkaline-hypersaline lake sediment]
MRRSHALKLDPNAGQARKLRLYCIAHCKIWNHFLEKCKKGLIPKKSNAMTHQLGNKRMDETCPELLWVRDELGMNKQIQQVPCEDL
EKAWKRFFDWCRARKKGNVGLRRVGPPKFKSSRWMVGMSFGAKQGVEHDPETRKVRIPSIGWIRHFEKDVDFSNDKIKCCRVVWRA
SGWYCSLSMDNDIEIKSPLKWKKRKTHSVHLGIRHYAVLSDGNNVDHPRNLERNLDRLSKLCREWSRKKKGSRRWRIWRDRVRRVH
EKIANCRKDFIHKMTDDLLKKYPCLISEKWSISDMVENEKTKRKRRKIKAGRRKPGSRMMEKLLHRGIMDSSWYEVRRQLKYKSEW
RRKPYHETHMGFKSTQTCFHCKHVNDFVTLNMKTFTCRGCGRKIERESNAAANVREMGFSELGL (SEQ ID NO: 322)
>3300017971|Ga0180438_10021976_11|M
[aquatic-non marine saline and alkaline-hypersaline lake sediment]
MTHQLGNKRMDETCPELLWVRDELGMNKQIQQVPCEDLEKAWKRFFDWCRARKKGNVGLRRVGPPKFKSSRWMVGMSFGAKQGVEH
DPETRKVRIPSIGWIRHFEKDVDFSNDKIKCCRVVWRASGWYCSLSMDNDIEIKSPLKWKKRKTHSVHLGIRHYAVLSDGNNVDHP
RNLERNLDRLSKLCREWSRKKKGSRRWRIWRDRVRRVHEKIANCRKDFIHKMTDDLLKKYPCLISEKWSISDMVENEKTKRKRRKI
KAGRRKPGSRMMEKLLHRGIMDSSWYEVRRQLKYKSEWRRKPYHETHMGFKSTQTCFHCKHVNDFVTLNMKTFTCRGCGRKIERES
NAAANVREMGFSELGL (SEQ ID NO: 323)
>3300025586|Ga0207996_1000986_11|P
[aquatic-non marine saline and alkaline-saline lake]
MTNRLRYSYRLYPTPEQRHALAQTFGCARVVWNDALARAQVGGAKYPGFAATSRLLTESKKSPARAWLNDVSCVPVQQSIRNLDIA
FRHFFNGLKGKGPKTGFPAWKRKDGKQSAEFTRRGFKLQRGKLCLAKIGDVEVRWSRSLPSAPKTATVTLDTAGRYHVSFTVEKVD
VAMEGGSPVGIDLGIKTFAALSTGELVHAPELKRLERRIDKAQRALSRCQKGSARRGRAKLRVAKLNATLADTRRDFLEKFSTRLV
RQHSLIALEDLAVANMTKNHCLARAVSRQGWRMFRTMVESKCERYGREIRIVNRWEPTSQVCSGCGHRWGKLGLGVREIACEGCGE
VHDRDINAAKNVVAAGLAETKNGRGEWVNRVVSMGTGVPFVEASTAGASPGVSAL (SEQ ID NO: 324)
>3300025642|Ga0208648_1003027_2|M
[aquatic-non marine saline and alkaline-saline lake]
MTNRLRYSYRLYPTPEQRHALAQTFGCARVVWNDALARAQVGGAKYPGFAATSRLLTESKKSPARAWLNDVSCVPVQQSIRNLDIA
FRHFFNGLKGKGPKTGFPAWKRKDGKQSAEFTRRGFKLQRGKLCLAKIGDVEVRWSRSLPSAPKTATVTLDTAGRYHVSFTVEKVD
VAMEGGSPVGIDLGIKTFAALSTGELVHAPELKRLERRIDKAQRALSRCQKGSARRGRAKLRVAKLNATLADTRRDFLEKFSTRLV
RQHSLIALEDLAVANMTKNHCLARAVSRQGWRMFRTMVESKCERYGREIRIVNRWEPTSQVCSGCGHRWGKLGLGVREIACEGCGE
VHDRDINAAKNVVAAGLAETKNGRGEWVNRVVSMGTGVPFVEASTAGASPGVSAL (SEQ ID NO: 325)
>3300028299|Ga0268276_1017374_5|P
[aquatic-non marine saline and alkaline-saline water]
MPMRWRWQTYTPRVRGVRVMKMHRAHRIRLNPTPEQEQYLWRCAGVARFTWNWALAAYNDTLARGEKPSIAKLKVEFNRLRAEESF
APWVGEVQSYAYQYAFGDLRTAINRYHKLRKADKLKPRAGWKPRKDGRPFGWPRFKARALTTPAFGLANNGGVHCAGNLAYLQRCP
SPINMAEELRFTGRILSGRVSYTGGHWYLSVSLDLPDPQPEPLSGRVGVDLGIKSLAVTSDGVVYENPKHLRANQRKLARLQRELS
RRTKGGKNRAKTQAKIARLHEQIANRRRETLHEMTTDLVSSYGTVVVEDLNVSGMLSNHRLALAVSDAAFGEIRRQLEYKAPAHGS
QVLVANQWFPSSKTCNDCGYITTLTLSDRVWTCPNCGVVHDRDINAAQNLRDYQPASAA (SEQ ID NO: 326)
>3300028299|Ga0268276_1017374_4|M
[aquatic-non marine saline and alkaline-saline water]
MKMHRAHRIRLNPTPEQEQYLWRCAGVARFTWNWALAAYNDTLARGEKPSIAKLKVEFNRLRAEESFAPWVGEVQSYAYQYAFGDL
RTAINRYHKLRKADKLKPRAGWKPRKDGRPFGWPRFKARALTTPAFGLANNGGVHCAGNLAYLQRCPSPINMAEELRFTGRILSGR
VSYTGGHWYLSVSLDLPDPQPEPLSGRVGVDLGIKSLAVTSDGVVYENPKHLRANQRKLARLQRELSRRTKGGKNRAKTQAKIARL
HEQIANRRRETLHEMTTDLVSSYGTVVVEDLNVSGMLSNHRLALAVSDAAFGEIRRQLEYKAPAHGSQVLVANQWFPSSKTCNDCG
YITTLTLSDRVWTCPNCGVVHDRDINAAQNLRDYQPASAA (SEQ ID NO: 327)
>SRR1221442_902393_36|P
[gut metagenome]
MKQAIRQMDGAYQKFFKQHNGFPKFKSKKDKQSALFPYEAISKHNTFETRHISLTTLLKNIRFRCSDLYFSRLQKYNKNIRSATLS
KTKSGNFFLSILIEMEDAELKRFGHTNKQVGIDLGVKDFVITSDGEVFENKHFFRKEERQVKKLHRQLSKKVKGSNNRKKTQVRIA
KLFERITNKKNAYIHYVTNELLTYFDTIFMEELNVKGMLRNHHLAKAIQEVGFYKFKETLVNKAFVNDKQVVFIDRFYPSSKTCSV
CGYKKQDLRLSDREWVCPKCGTKHNRDINAAVNILLEGQRMLTAG (SEQ ID NO: 328)
>3300001598|EMG_10002624_36|P
[mammals-digestive system-asian elephant f ecal-elephas maximus]
MKQAIRQMDGAYQKFFKQHNGFPKFKSKKDKQSALFPYEAISKHNTFETRHISLTTLLKNIRFRCSDLYFSRLQKYNKNIRSATLS
KTKSGNFFLSILIEMEDAELKRFGHTNKQVGIDLGVKDFVITSDGEVFENKHFFRKEERQVKKLHRQLSKKVKGSNNRKKTQVRIA
KLFERITNKKNAYIHYVTNELLTYFDTIFMEELNVKGMLRNHHLAKAIQEVGFYKFKETLVNKAFVNDKQVVFIDRFYPSSKTCSV
CGYKKQDLRLSDREWVCPKCGTKHNRDINAAVNILLEGQRMLTAG (SEQ ID NO: 329)
>ORBV01001191_65|M
[microbial mat metagenome]
MNDVSSVPVQQSIRNLDVAFRRFFNGLKGKGPKVGFPTWKRKDGKQSAEFTRSGFKLQRGKLYIAKVGDVDVRWSRALPSVPKTAT
VTLDAAGRYHVSFTVEKTDVALNGGSPVGVDLGIKTFAALSTGELVHAPDTKRLERRIGKAQRGLSRCRKGSARRGRAKLRVAKLH
AVLADTRRDFLEKFSTRLVREHSLIALEDLNVAGMAKNHCLARAVNRQGWRMFRTMVQSKCERYGREVRIVGRWEPTSQVCSGCGH
RWGRVGLGVRTIACEGCGEVHDRDVNAAKNVVAAGLADTENGRGEWVSRVVSMGTGVPFVEASTAGASPGISAL (SEQ ID NO:
330)
>ORBV01001191_63|P
[microbial mat metagenome]
MASLRYSYRLYPTTEQRRALAQTFGCVRVAWNDALARSQVSGAKYPGFAATSRLLTESKKSPERAWLNDVSSVPVQQSIRNLDVAF
RRFFNGLKGKGPKVGFPTWKRKDGKQSAEFTRSGFKLQRGKLYIAKVGDVDVRWSRALPSVPKTATVTLDAAGRYHVSFTVEKTDV
ALNGGSPVGVDLGIKTFAALSTGELVHAPDTKRLERRIGKAQRGLSRCRKGSARRGRAKLRVAKLHAVLADTRRDFLEKFSTRLVR
EHSLIALEDLNVAGMAKNHCLARAVNRQGWRMFRTMVQSKCERYGREVRIVGRWEPTSQVCSGCGHRWGRVGLGVRTIACEGCGEV
HDRDVNAAKNVVAAGLADTENGRGEWVSRVVSMGTGVPFVEASTAGASPGISAL (SEQ ID NO: 331)
>OVSM01005865_8|M
[seawater metagenome]
MRWWMVRLKESQEYSFLRELNSQSLVSRLRELDNTYWDCFLKRKEGAQLPVIKKRSKHNSFHLPDPKKQAQVKGNKVKLSKIGEVT
FFKSRKIDGEIRTARVFYRTGYWYISFQVKQEIDIPKRKDKGRAGIYLGGKNLLTLVTNKNKKERYDLLNIYGKYEPLLKKKQKSL
KRKVSFSKNWEKKQAEIKKIHRKIFNTRRDTLHKISTEVCKNHATIYLSDFRVLKTLNKGRLKKKEKEKNKKVLDQGLWYLKEFIV
YKGLWRGNIVKKVNNENSILTCSSCGVINDTRQGEKFCCNDCGLKESLEINAAKNILAAGHAVTVCEANQLRN (SEQ ID NO:
332)
>OVXX01005191_2|P
[seawater metagenome]
MRWWMVRLKESQEYSFLRELNSQSLVSRLRELDNTYWDCFLKRKEGAQLPVIKKRSKHNSFHLPDPKKQAQVKGNKVKLSKIGEVT
FFKSRKIDGEIRTARVFYRTGYWYISFQVKQEIDIPKRKDKGRAGIYLGGKNLLTLVTNKNKKERYDLLNIYGKYEPLLKKKQKSL
KRKVSFSKNWEKKQAEIKKIHRKIFNTRRDTLHKISTEVCKNHATIYLSDFRVLKTLNKGRLKKKEKEKNKKVLDQGLWYLKEFIV
YKGLWRGNIVKKVNNENSILTCSSCGVINDTRQGEKFCCNDCGLKESLEINAAKNILAAGHAVTVCEANQLRN (SEQ ID NO:
333)
>3300005549|Ga0070704_100000680_37|P
[terrestrial-soil-corn, switchgrass and miscanthus rhizosphere]
MPGDAPAGHVYLRAFQTELDPTVAERRLLGRHVAAARVSHNWALERWNSLQCPRAIVRGLRALAGLDVKGGEAANMLGVFLRALVF
GTPVAVRTPKGAARRYRYKPPEPPDVDVSPNGPSIHASLVREKNNAQDLAWLGEVSAFAVREAVEDVADGWKHFFEHLKAGRFDKA
GPPQFRKARDRHYHVDQPDPIRVTDRAVKIPGVGWVRLKERGYLPPTRADSHALVGGGKCVGLGISERDGRWYVSLRAHVPRPMPQ
KRAPGRAKRDAPVPRNPNLTLGVEVGVRSLVTLSTGAHFAGLREDERIIAATRRLHLWQRRQERRWQRGKSRREQSAGWREAVRWV
GHYHAQLTDLRADLTGKAVRAIVDTGAAVIVMREPATAKMLARRHASEPRTRNALAPAVHAARMGDVLRRVGYKQKWAGGQLVEVP
VAEPVTRRCNKCGAVRDTEPAYPDFHCTACGHREERDDNAAANLKDYPGRNPGADPGRSGPDSPSAHTEGVTAGGTAGTASGESPG
HGSGRKARERASLGPGNRPRAGARLTAQADQPLTSPRYDGALREHRGDAPEQSQGNHTPAAPVPADDGDRSQTVSQPRERTAELRG
VSGAQP (SEQ ID NO: 334)
>3300005549|Ga0070704_100000680_28|M
[terrestrial-soil-corn, switchgrass and miscanthus rhizosphere]
MKLKDGGNIMPGDAPAGHVYLRAFQTELDPTVAERRLLGRHVAAARVSHNWALERWNSLQCPRAIVRGLRALAGLDVKGGEAANML
GVFLRALVFGTPVAVRTPKGAARRYRYKPPEPPDVDVSPNGPSIHASLVREKNNAQDLAWLGEVSAFAVREAVEDVADGWKHFFEH
LKAGRFDKAGPPQFRKARDRHYHVDQPDPIRVTDRAVKIPGVGWVRLKERGYLPPTRADSHALVGGGKCVGLGISERDGRWYVSLR
AHVPRPMPQKRAPGRAKRDAPVPRNPNLTLGVEVGVRSLVTLSTGAHFAGLREDERIIAATRRLHLWQRRQERRWQRGKSRREQSA
GWREAVRWVGHYHAQLTDLRADLTGKAVRAIVDTGAAVIVMREPATAKMLARRHASEPRTRNALAPAVHAARMGDVLRRVGYKQKW
AGGQLVEVPVAEPVTRRCNKCGAVRDTEPAYPDFHCTACGHREERDDNAAANLKDYPGRNPGADPGRSGPDSPSAHTEGVTAGGTA
GTASGESPGHGSGRKARERASLGPGNRPRAGARLTAQADQPLTSPRYDGALREHRGDAPEQSQGNHTPAAPVPADDGDRSQTVSQP
RERTAELRGVSGAQP (SEQ ID NO: 335)
>3300010400|Ga0134122_10000002_228|P
[terrestrial-soil-terrestrial soil]
MTTILRAYKTELDPTDEQRVVLARHVAGARVAHNWALEKWKELDGTRGVALGCRALAGIDAKGGEAAALFGYCLAALIRGEPVKVR
TPKSEAGSRYRYRPASGVQLPDFRAGRVDWYSQLVSAKEEQPERFGWLSELSAFAIREAVLDVADGWKHFFEHLKAGRYASAGPPR
FRGRQRASYHSDQPDPIRVTERAVKIPGVGWVRMKERGYLPVTEENSHRFIGGGKACGLGVSEKDGRWYVALRCEVPRPFPQKRGP
GRALAMHQTKRVPGRELGVEVGVRVLAVASTGERAGHVWHSPLRDDRRIDKFEMLRKRWERRMARRWKDGVKTRDQSQGWKEAAMR
VAHYHARIKSLRDDVCGKAARQIADMGAETVRMRGPGIAKMLSRAGKRGDAARTRNRLAKNVHAARMGDLRRRLEYKQAWAGGRLE
LVPIEEPTTNRCSACNAVRDTNAGYPTFMCPTCGYQEDRDDNSAINLLNYRAPSGDPAAGPRSAGLKPPKGDNGRRKRTARAEEKS
SVTAPHGDVTSAPGPGNRVSGASHSVRPIDAYHEVPFEQSGDAHLGSRQFGLDTALFGADDPNRSQIEPQRSEFPGLLAHRAGGTS
(SEQ ID NO: 336)
>3300009089|Ga0099828_10012481_2|M
[terrestrial-soil-vadose zone soil]
MKQLCAMQEALRDLETAYRNFFRRVALGKQGLWRGKLGFPKMKKKSKAIGSFRLTGSIKVFSGTVQLPRLGCLRLHEHDFIPTDAK
VLSATVSEQAGRWFVSIQVEEEQEKPPPTATTAIGVDLGIKTLATCSDGKIFANPRALSHAHKMLRRLERQKSRRKKGGKNRKKIC
RKLAKQHARVANIRKDAAHKLSSYLCKNHALVAIEDLHVAGMLKNHKLAQAVSDSNFGEIRRQLEYKAFWQGVHLVIIDRWYASSK
TCSGCGWVDENQDLSDRTFICQECGLVMDRDENAAINILNEALRTTGSYSGSHAYGESSSGLVSGSGETALVEIGTNLHVGMS
(SEQ ID NO: 337)
>3300012201|Ga0137365_10010044_6|P
[terrestrial-soil-vadose zone soil]
MKRAAPLESGMIRRAYRVRLEPTAEQRRELARCVGVARAAFNWALAEWRRQYRAYKLAEKAPARALRLLARWQSPLAEDPRWQGGD
NPKPNGFAIHKRLTAIKREQFPWMTDVPALVVREAVGDVGAAYEHFFRRLKEGAVGREAGEPRFRSRHRRKSFHMDQGDAIKIARF
VRADETTGRHADAIVLPVLGSVHVHKRQNGFLPAGAKLCGVGISEHLGLWYAAVRAEVPAPAPRRKRMAEKRLGVEVGVRSLAVTS
DGKRIGALRDLDKTKSGERRLALWLRRMSRRFRQGAKKQSAGWVEAKHHVQRLHSEIAETRDELLHYASRRVVDSGAAVVVMREPN
VRGMIGKAGKTGEAARARNVIAPMVSKVGWYELRRKVEYKQQWAGGAFVEAPAEIESTKTCSVCGSVRDTDPGYPLFRCPSCGHQE
DREANSSKVLRDYRPPSGGPVGGDRADRRKTARKRGTMAERTGKPGGEQSAPTGPDGVATSPHGSGNGAASQEAETGAVIRDPTRP
PGEPGARLRVPSATASDGGHEPIDSQESCVFASPPDGPDGDRSQTASQASETSGGSA (SEQ ID NO: 338)
>3300012356|Ga013737l_10006599_2|M
[terrestrial-soil-vadose zone soil]
MKRAAPLESGMIRRAYRVRLEPTAEQRRELARCVGVARAAFNWALAEWRRQYRAYKLAEKAPARALRLLARWQSPLAEDPRWQGGD
NPKPNGFAIHKRLTAIKREQFPWMTDVPALVVREAVGDVGAAYEHFFRRLKEGAVGREAGEPRFRSRHRRKSFHMDQGDAIKIARF
VRADETTGRHADAIVLPVLGSVHVHKRQNGFLPAGAKLCGVGISEHLGLWYAAVRAEVPAPAPRRKRMAEKRLGVEVGVRSLAVTS
DGKRIGALRDLDKTKSGERRLALWLRRMSRRFRQGAKKQSAGWVEAKHHVQRLHSEIAETRDELLHYASRRVVDSGAAVVVMREPN
VRGMIGKAGKTGEAARARNVIAPMVSKVGWYELRRKVEYKQQWAGGAFVEAPAEIESTKTCSVCGSVRDTDPGYPLFRCPSCGHQE
DREANSSKVLRDYRPPSGGPVGGDRADRRKTARKRGTMAERTGKPGGEQSAPAGPDGVATSPHGSGNGAASQEAETGAVIRDPTRP
PGEPGARLRVPSATASDGGHEPIDSQESCVFASPPDGPDGDRSQTASQASETSGGSA (SEQ ID NO: 339)
>3300006417_501592_1|M
[wastewater metagenome]
MLKAHVIRLNPTEEQASYFWRCAGIARFTWNWALAELNAAYDRGERPAIGSLKLAFNRLRKEEGFAPFVGEVQSYAYQQAFTDLQK
ALSRYHDFRKRGLLKPPAGWKGRKDHKPFGWPRFKARNRSTPAFYLANNGGLRLQGHQVTIQRCPGGPVNMAEQLRFAGRVMGGRV
RYRAGHWYLTVQVDVPVEPVPAHTGPAVGLDVGIKALAVTSDGEIYDNPKALGRHQRKLRLLQRSLARQTRGGSNYRKTQAKIARL
HERIANIRKHTLHQISHEITRDYGLIGLEDLNVAGMLENGKLARSISDVAFGELRRQIGYKSEWRGSRVVIVSRWFPSSKTCNECG
HVMADMPLSVRWWQCPTCGAEHDRDGNAAVNIRNEAVKMAGAA (SEQ ID NO: 340)
>SRR6837571_682758_2|M
[wastewater metagenome]
MLKAHVIRLNPTEEQASYFWRCAGIARFTWNWALAELNAAYDRGERPAIGSLKLAFNRLRKEEGFAPFVGEVQSYAYQQAFTDLQK
ALSRYHDFRKRGLLKPPAGWKGRKDHKPFGWPRFKARNRSTPAFYLANNGGLRLQGHQVTIQRCPGGPVNMAEQLRFAGRVMGGRV
RYRAGHWYLTVQVDVPVEPVPAHTGPAVGLDVGIKTLAVTSDGEIYDNPKALGRHQRKLRLLQRSLARQTRGGSNYRKTQAKIARL
HERIANIRKHTLHQISHEITRDYGLIGLEDLNVAGMLKNGKLARSISDVAFGELRRQIGYKSEWRGSRVVIVSRWFPSSKTCNECG
HVMADMPLSVRWWQCPTCGAEHDRDGNAAVNIRNEAVKMAGAA (SEQ ID NO: 341)

TABLE 182
Nucleotide Sequences of Representative CLUST.342201 Direct Repeats and Spacer
Lengths

| | | Spacer |
|---|---|---|
| CLUST.342201 Effector Protein | | Length |
| Accession | Direct Repeat Nucleotide Sequence | (s) |
| 3300006417\|Ga0069787_ | GTTCACCCCACGGGTGCGTGGAGTGATGG (SEQ ID NO: 342) | 32-33 |
| 10357475_9\|M (SEQ ID NO: 301) | CCATCACTCCACGCACCCGTGGGGTGAAC (SEQ ID NO: 451) | |
| 3300022602\|Ga0248169_134333_ | GTTCGCCGCGCATACGCGGCTTAAAAG (SEQ ID NO: 343) | 33 |
| 3\|P (SEQ ID NO: 302) | CTTTTAAGCCGCGTATGCGCGGCGAAC (SEQ ID NO: 452) | |
| 3300022602\|Ga0248169_134333_ | GTTCGCCGCGCATACGCGGCTTAAAAG (SEQ ID NO: 343) | 33 |
| 4\|M (SEQ ID NO: 303) | CTTTTAAGCCGCGTATGCGCGGCGAAC (SEQ ID NO: 452) | |
| 3300025018\|Ga0210043_1027795_ | GGTTACCCCGCGCGGGTGTGGAGTGAGGG (SEQ ID NO: 344) | 32-33 |
| 3\|M (SEQID NO: 304) | CCCTCACTCCACACCCGCGCGGGGTAACC (SEQ ID NO: 384) | |
| 3300020198\|Ga0194120_10001032_ | CTTGCGGGATCGGTTTGAATGGATAGGAAAGTGACAT (SEQ ID NO: | 33-36 |
| 39\|P (SEQ ID NO: 305) | 345) | |
| | ATGTCACTTTCCTATCCATTCAAACCGATCCCGCAAG (SEQ ID NO: | |
| | 385) | |
| 3300014060\|Ga0119967_10019787_ | GGTTACCCCGCGCGGGTGTGGAGTGAGGG (SEQ ID NO: 344) | 32 |
| 9\|M (SEQ ID NO: 306) | CCCTCACTCCACACCCGCGCGGGGTAACC (SEQ ID NO: 384) | |
| 3300020180\|Ga0163155_10006838_ | CAGCGCACGAGGAGGTGCTG (SEQ ID NO: 346) | 22 |
| 6\|M (SEQ ID NO: 307) | CAGCACCTCCTCGTGCGCTG (SEQ ID NO: 386) | |
| 3300020180\|Ga0163155_10006838_ | CAGCGCACGAGGAGGTGCTG (SEQ ID NO: 346) | 22 |
| 8\|P (SEQ ID NO: 308) | CAGCACCTCCTCGTGCGCTG (SEQ ID NO: 386) | |
| 3300020219\|Ga0163146_10008638_ | CTGCCATGGTGGCGTGGTCGGGAAGTCCCCATGGCATGGGCTCTGTCTCAT | 20-21 |
| 12\|M (SEQ ID NO: 309) | TCGGCGAACCGCCC (SEQ ID NO: 347) | |
| | GGGCGGTTCGCCGAATGAGACAGAGCCCATGCCATGGGGACTTCCCGACCA | |
| | CGCCACCATGGCAG (SEQ ID NO: 387) | |
| 3300020219\|Ga0163146_10008638_ | CTGCCATGGTGGCGTGGTCGGGAAGTCCCCATGGCATGGGCTCTGTCTCAT | 20-21 |
| 11\|P (SEQID NO: 310) | TCGGCGAACCGCCC (SEQ ID NO: 347) | |
| | GGGCGGTTCGCCGAATGAGACAGAGCCCATGCCATGGGGACTTCCCGACCA | |
| | CGCCACCATGGCAG (SEQ ID NO: 387) | |
| 3300001380\|JGI1356J14229_ | GGCACAGTGGTTGGCTTCTTCTTCCCA (SEQ ID NO: 348) | 21 |
| 10025169_2\|P (SEQ ID NO: 311) | TGGGAAGAAGAAGCCAACCACTGTGCC (SEQ ID NO: 388) | |
| 3300014613\|Ga0180008_1001166_ | GTACACCCCACGGGTGCGTGGAGTGAGGG (SEQ ID NO: 349) | 32-33 |
| 14\|P (SEQ ID NO: 312) | CCCTCACTCCACGCACCCGTGGGGTGTAC (SEQ ID NO: 389) | |
| 3300014613\|Ga0180008_1001166_ | GTACACCCCACGGGTGCGTGGAGTGAGGG (SEQ ID NO: 349) | 32-33 |
| 12\|M (SEQ ID NO: 313) | CCCTCACTCCACGCACCCGTGGGGTGTAC (SEQ ID NO: 389) | |
| 3300014656\|Ga0180007_10002258_ | GTACACCCCACGGGTGCGTGGAGTGAGGG (SEQ ID NO: 349) | 32-33 |
| 14\|P (SEQ ID NO: 314) | CCCTCACTCCACGCACCCGTGGGGTGTAC (SEQ ID NO: 389) | |
| 3300014656\|Ga0180007_10002258_ | GTACACCCCACGGGTGCGTGGAGTGAGGG (SEQ ID NO: 349) | 32-33 |
| 12\|M (SEQ ID NO: 315) | CCCTCACTCCACGCACCCGTGGGGTGTAC (SEQ ID NO: 389) | |
| 3300013127\|Ga0172365_10076875_ | GTCGGGCCCACGCGTACGTGGGCGATTGG (SEQ ID NO: 350) | 32-39 |
| 1\|P (SEQ ID NO: 316) | CCAATCGCCCACGTACGCGTGGGCCCGAC (SEQ ID NO: 390) | |
| 3300007960\|Ga0099850_1017825_ | ATACGCCCCACGTGCGTGTGGGGTGAGGG (SEQ ID NO: 351) | 32-33 |
| 5\|M (SEQ ID NO: 317) | CCCTCACCCCACACGCACGTGGGGCGTAT (SEQ ID NO: 391) | |
| 3300025687\|Ga0208019_1031224_ | ATACGCCCCACGTGCGTGTGGGGTGAGGG (SEQ ID NO: 351) | 32-33 |
| 1\|P (SEQ ID NO: 318) | CCCTCACCCCACACGCACGTGGGGCGTAT (SEQ ID NO: 391) | |
| 3300025687\|Ga0208019_1031224_ | ATACGCCCCACGTGCGTGTGGGGTGAGGG (SEQ ID NO: 351) | 32-33 |
| 1\|M (SEQ ID NO: 319) | CCCTCACCCCACACGCACGTGGGGCGTAT (SEQ ID NO: 391) | |
| 3300017963\|Ga0180437_10001136_ | CGTGTGAATGCGTGAAATTGAGTAGAGGATATGTCAG (SEQ ID NO: | 28-37 |
| 84\|M (SEQ ID NO: 320) | 352) | |
| | CTGACATATCCTCTACTCAATTTCACGCATTCACACG (SEQ ID NO: | |
| | 392) | |
| 3300017963\|Ga0180437_10001136_ | CGTGTGAATGCGTGAAATTGAGTAGAGGATATGTCAG (SEQ ID NO: | 28-37 |
| 87\|P (SEQ ID NO: 321) | 352) | |
| | CTGACATATCCTCTACTCAATTTCACGCATTCACACG (SEQ ID NO: | |
| | 392) | |
| 3300017971\|Ga0180438_10021976_ | GTGTGAATGCGTGAAATTGAGTAGAGGATATGTCAG (SEQ ID NO: | 35-36 |
| 13\|P (SEQ ID NO: 322) | 353) | |
| | CTGACATATCCTCTACTCAATTTCACGCATTCACAC (SEQ ID NO: | |
| | 393) | |
| 3300017971\|Ga0180438_10021976_ | GTGTGAATGCGTGAAATTGAGTAGAGGATATGTCAG (SEQ ID NO: | 35-36 |
| 11\|M (SEQ ID NO: 323) | 353) | |
| | CTGACATATCCTCTACTCAATTTCACGCATTCACAC (SEQ ID NO: | |
| | 393) | |
| 3300025586\|Ga0207996_1000986_ | CGCAAGCCGAAGGCTTACGGATGCGG (SEQ ID NO: 354) | 15-50 |
| 11\|P (SEQ ID NO: 324) | CCGCATCCGTAAGCCTTCGGCTTGCG (SEQ ID NO: 394) | |
| 3300025642\|Ga0208648_1003027_ | CTGCGCAAGCCGAAGGCTTACGGATGCGG (SEQ ID NO: 355) | 12-18 |
| 2\|M (SEQ ID NO: 325) | CCGCATCCGTAAGCCTTCGGCTTGCGCAG (SEQ ID NO: 395) | |
| 3300028299\|Ga0268276_1017374_ | GTACACCCCACGGGTGCGTGGAGTGAGGG (SEQ ID NO: 349) | 32 |
| 5\|P (SEQ ID NO: 326) | CCCTCACTCCACGCACCCGTGGGGTGTAC (SEQ ID NO: 389) | |
| 3300028299\|Ga0268276_1017374_ | GTACACCCCACGGGTGCGTGGAGTGAGGG (SEQ ID NO: 349) | 32 |
| 4\|M (SEQ ID NO: 327) | CCCTCACTCCACGCACCCGTGGGGTGTAC (SEQ ID NO: 389) | |
| SRR1221442_902393_361P | TCTTCATCAACCTTCACATCTAC (SEQ ID NO: 356) | 17-20 |
| (SEQ ID NO: 328) | GTAGATGTGAAGGTTGATGAAGA (SEQ ID NO: 396) | |
| 33000015981 EMG_10002624_ | TCTTCATCAACCTTCACATCTAC (SEQ ID NO: 356) | 17-20 |
| 36\|P (SEQ ID NO: 329) | GTAGATGTGAAGGTTGATGAAGA (SEQ ID NO: 396) | |
| ORBV01001191_65\|M | CTGCCATGGTGGCGTGGTCGGGAAGTCCCCATGGCATGGGCTCTGTCTCAT | 20-21 |
| (SEQ ID NO: 330) | TCGGCGAACCGCCC (SEQ ID NO: 347) | |
| | GGGCGGTTCGCCGAATGAGACAGAGCCCATGCCATGGGGACTTCCCGACCA | |
| | CGCCACCATGGCAG (SEQ ID NO: 387) | |
| ORBV01001191_63\|P | CTGCCATGGTGGCGTGGTCGGGAAGTCCCCATGGCATGGGCTCTGTCTCAT | 20-21 |
| (SEQ ID NO: 331) | TCGGCGAACCGCCC (SEQ ID NO: 347) | |
| | GGGCGGTTCGCCGAATGAGACAGAGCCCATGCCATGGGGACTTCCCGACCA | |
| | CGCCACCATGGCAG (SEQ ID NO: 387) | |
| OVSM01005865_8\|M | AATATTACACATAAAACTACATACATAATTACATA (SEQ ID NO: | 25 |
| (SEQ ID NO: 332) | 357) | |
| | TATGTAATTATGTATGTAGTTTTATGTGTAATATT (SEQ ID NO: | |
| | 397) | |
| OVXX01005191_2\|P | AATATTACACATAAAACTACATACATAATTACATA (SEQ ID NO: | 25 |
| (SEQ ID NO: 333) | 357) | |
| | TATGTAATTATGTATGTAGTTTTATGTGTAATATT (SEQ ID NO: | |
| | 397) | |
| 3300005549\|Ga0070704_ | CCGTGACGGGGCGTCGTGGTCAG (SEQ ID NO: 358) | 49-50 |
| 100000680_37\|P (SEQ ID NO: 334) | CTGACCACGACGCCCCGTCACGG (SEQ ID NO: 398) | |
| 3300005549\|Ga0070704_ | CCGTGACGGGGCGTCGTGGTCAG (SEQ ID NO: 358) | 49-50 |
| 100000680_28\|M (SEQ ID NO: 335) | CTGACCACGACGCCCCGTCACGG (SEQ ID NO: 398) | |
| 3300010400\|Ga0134122_ | TCGTGACGGGCGGCGCTTGTCAG (SEQ ID NO: 359) | 36-55 |
| 10000002_228\|P (SEQ ID NO: 336) | CTGACAAGCGCCGCCCGTCACGA (SEQ ID NO: 399) | |
| 3300009089\|Ga0099828_ | TTGATCAGATCATCGAGTTGAGTGAGCACGAGGT (SEQ ID NO: 360) | 52-62 |
| 10012481_2\|M (SEQ ID NO: 337) | ACCTCGTGCTCACTCAACTCGATGATCTGATCAA (SEQ ID NO: 454) | |
| 3300012201\|Ga0137365_ | CTTGCAATGCGCGAAGGAAAGCGTCGACGTGGTCAC (SEQ ID NO: | 29-40 |
| 10010044_6\|P (SEQ ID NO: 338) | 361) | |
| | GTGACCACGTCGACGCTTTCCTTCGCGCATTGCAAG (SEQ ID NO: | |
| | 401) | |
| 3300012356\|Ga0137371_ | CTTGCAATGCGCGAAGGAAAGCGTCGACGTGGTCAC (SEQ ID NO: | 29-40 |
| 10006599_2\|M (SEQ ID NO: 339) | 361) | |
| | GTGACCACGTCGACGCTTTCCTTCGCGCATTGCAAG (SEQ ID NO: | |
| | 401) | |
| 3300006417_501592_1\|M (SEQ ID | GTTCACCCCACAGGCGCGTGGAGTGATGG (SEQ ID NO: 362) | 32 |
| NO: 340) | CCATCACTCCACGCGCCTGTGGGGTGAAC (SEQ ID NO: 402) | |
| SRR6837571_682758_2\|M (SEQ ID | GTTCACCCCACAGGCGCGTGGAGTGATGG (SEQ ID NO: 362) | 32 |
| NO: 341) | CCATCACTCCACGCGCCTGTGGGGTGAAC (SEQ ID NO: 402) | |

TABLE 193
Non-coding Sequences of Representative CLUST.342201 Systems

>3300013127|Ga0172365_10076875_1|P
GTTAGCACACAAGCGATAGCACATGACCGCGCCGAGGAGAGCGCAAGCTCTGCCAAACCCTCGGCGCGGTTTTTGTGCGCCTATGC
AGCCCGCCCTAATAACGTATATTATCGGACGTTATCCAGGGGCGGTTCGGGTCAGAAGCGGTCAGAAAAATGACGGCAATTTCGAT
TTCGCACAAGATTGCGCTTGAGCCTACGAAATTGCAGGCTGCCCACTTCGCGCTGTGCGTCGGTGCCTCGCGAAAGGCATACAATT
GGGCGCTGGATACGTGGAATCGCCAGTGGGACGATGGCAGAAAGCCGTGTTCGCGGTGCGGCCGCCGCAACTATGATGCGCGGAAG
TATGGCAAGCGATTTGTATGCCCGCATTGCGGGCTGGACATCGAGAACGGCGTGAACATTGCGTCGAATCTCGCTACCGTCAAATC
GGCGGAAAGTCAAGCCTGTGGATAGGCCGGCGTCAACCCCACGCTGCCCGTGAACGTGGCGCTACAATGGCCGAGAAGCAGGAACC
CGTGTACGCGTGGTCTTTGATATTTTGTGTGGGCTTGGACTGGGCACCACAAGATGTGGTCTTCAAGCGGCTCGTTCGGTGCGGGC
GGCATTTAACAATCTGACAACCCGCAGTCTGTAGCCGCCCGCGCCGGATGTTGAGGAGTAAACAATGGACACTCTAAGCGTGATCG
TAAATTGTGTAGCGCTTGCGGTCGTGGCCGGTGCTCTCGTTTACCTCGGATGGGCGCTCTACCAAGCGTATAGGAGGCGAATATGA
TGTTCTTAACTGATCTGCTTCACGATGCGGA (SEQ ID NO: 363)
>OVSM01005865_8|M
AAAACTACATACGATATTACATATAATTTATAACATAATATTACACACAAACTACATACATAATTACATACAATTTACAACATAGT
ATTACATACAAAACTACACATGATATTACATATTTTTATAAACCTATAAAACAAAATTCCTTCTTATAAACAAGATTACCCTAGTT
GATGATATTATACACAACACCACATTATGTCATAGAAATATATATCTTTCATTTTTCTGTAAAAAAACACTAGGTTTGTATAGTTT
TTTGCAAAAAAACGTTCCCCCCCTAAAATAATTTTTTTCTTCTAAAAGAATTTTTTTGCCTTTACAAGTTACCACTGTTAATGCAT
GATGAACTTAAACTTAAAAAATGGTGAAATATATTTTGTAAAAAACCTTCAAAAAGACAAAAAAAAAGAGCTAGACTCTTGCTTCA
AAGACAAAACCAAGAAACAAAAATCTAATCTCTCTAACATCAAGATTATGATACTATATTTCAACAGAACTATTTTAGTTATGGGT
AGTGATTTTAAGGTGTCTTATGTGAAAGCGACCTTTTGGAGAGAGCTTGAAGAGCTTAAGAAATCCTTTTTTTCTACAAGAGACTT
TTCTGTAGAAAAGAGAGAAGCACACCAACAAGGGAGTTAAGTGTGCTTCTAATAATGAAAATCTACTAACAGATTCGCGTTCCATT
TTACTCTAAAAAGATAATTTTAACAAATAGGAAAAAATATTTCTTTAATTGAGAAAATTTACATTTCTGGTTACAATGATCATTGC
AAAAGGGATGCGAAAATAGGTTTTTAAGTGATAATCCACAAAACATATCAGTTTAGATTAAAGCCAACAAAAGAACAGGAGGCTTT
GTTTTGGCGGTTTTCCGGTCATTGCAGGTTTATCTGGAATTACTTTTGGAAAATAAATAAAATCAGACTCGATAACAAACAAAAAA
TAATGAAGTTCTACGAAATGCGCTGGTGGATGGTTCGTCTGAAAGAGTCGCAGGAATATAGCTTTTTAAGAGAATTAAATTCTCAG
TCTTTAGTTTCTCGTTTGAGGGAGTTGGATAATACGTATTGGGATTGTTTTTTAAAGAGAAAAGAAGGAGTTCAACTTCCTTGTGG
CGTTATTAATGATACCAGACAAGGAGAAAAATTTTGTTGCAATGATTGCGGTTTAAAAGAGTCGCTAGAGATAAATGCAGCAAAAA
ATATATTGGCGGCAGGGCATGCCGTGACGGTCTGTGAAGCGAATCAATTAAGAAATTAAGAGATCGGCAGCAGAAACCTGTAGAGA
TATGTGAATATATCTACCTTAGAAATAGGAATCTCATACCGTAAAGGGAAGAGAGGATGCCAACCTGCGTATTTTAATCTATATTT
GGAGAACTTGCTCTAAATGAAAAAGGAAGTTAGCATAGGAAATGTAAATAGTATTAAGACAGGAAAAATATCAATGAATAAATCTT
TAAGAAATCTAATTGTCATTCTTTCTTTTTTTGCTTTATCTGGCTGCCAGAATAAAGATATAAACACTACAGGTAATTCT (SEQ
ID NO: 364)
>3300014613|Ga0180008_1001166_12|M
GCCGATGAAACGCTCAAGGCTTGTATTGCCTGGCCGCTGTTGACCCTGGCTGATCTGGAGACGTTGCTTTCGGTAATAAACGAGGC
GTGCGAAACCCGATTCACCATCATCTCCGAGATGCAGAAACGCAATCGGCGCGCCCGCATTTGATCGAAAGTGTAGGAAGTTGCGA
ATAACTATTGACATTTTAGCAGAGGTGTGCTAGAATACATTTGCTTGGAACTGACAAGCCAAGTAGTTATAAATGGACTCTCCGCT
AAGACTCCTGCTCCGAAAGTTGGGGCCGACTTGCCTTAGCGCCTGTCAAGCGCAAATGCGGAGAGTCGGCGGTCGGAACCAACTTT
TAGCGCAGGAGTTTCTTTGTCTACAAATATCGCCGAAATACTCATGCGGAGATGTAAAACGCCTGGGGATGGTCGAGGTTCAACTA
AACCAGATGAACCAGGAATCCATACACTCGTTAGGAGCGAGAGAAATGCATAGAGCACATCGAATCCGATTGAATCCAACGCCGGA
ACAGGAAAAATATCTCTGGCGTTGCGCTGGCGTAGCCCGCTTCACCTGGAATTGGGCGCTGGCAGCTTACAATGACACTCTGGTCC
GCGGCGAGAAGCCATCAATTGCCAAGCTCAAAGTCAAATTCAATCGCAGTAGCAAAACGTGCAACGACTGCGGTTGCGTCACTGCG
CTAACGTTGTCTGACCGCGTATGGACATGCCCGAATTGTGGCGTTGTCCATGATCGAGATATCAATGCTGCGCAAAACCTGCGTGA
TTATCAGCCCGTTAGCGTAGCTTGATGGAATACAATATGTCGCCGGAGTACTCACCTGGAGACGCTAAACGCCTAGGGATGGTAGT
GGTAGTAGTCACGCCAAACGAACTAGGAATCTACGTGCTCCGTAAGCGCGTGGAGTGAGGGAAACGGGTGCGTCTACACCGAACGG
TGAACGCTTCCCGCCAGACACAGAAAACACCTGGCGCTCCGGCTTGACAGCCTGAGCGTTCCGTCGGTTTGCCGAAAGGCAGTGAG
CTTCGCACGAGTGCCGCCAACGCGGCCACCCTGTAAATTCAACCACCTGGAAGGGTCTGAATGCGAGTGCCCATCCCGCCAAGGGG
TGGGTCTGCCCTGTTCTCGCCAACCTTATCGTGTAATTCCAATCCTCTTGCCAGCCTATGGCCTCTGCACATCCTCCAATTGCTGA
AGAATGTAACTGGTCGTGTTTCCCTGTAAATTGGTTTCCAAATCCACGATATTGTCTATTGACAAACGGTAAAACCGTGATATAAT
GGGTTTAGGATGAAACGAAAGCAACGGGCACATGAAGGGAATCGCAAATGGAACACAGCGAAACTAGAGAAGAGCTGATCCAAGGC
ATTACCATGAATACTCAGGCTTGGCTGGCAGTGTTCTGAACCGGCAGGGTTGCGCAACAAGACCCGCTATCGCAAGCCGACTGCAC
CTTACGCAGAATCACTACAACTCGCGCGGACTTTCAACCAACACAGGAGGATAACCGCCATGGAACCCAAAATCAGCCAGTGCGAA
TGCGCCAACTGTGGCCGGTGCTGGAAA (SEQ ID NO: 365)
>3300020198|Ga0194120_10001032_39|P
TATGTAATAACTGAGAGACAGCTTAGGATAATTGAGGATGAGCTATTTAATATAAAAGATTTGGTTTCTAGTTTAGAGGATAGTGA
TATAAAATTTAGTATAGAAAAAAGTGTAGATAGGCTGATAGAAAGTATGTTTGATGGAGGATGATCTGCGAGGGCTCTGGTGCTGT
TCCAAATCTTAGGAACCTCTCGCGGTAGGCGGTGACTGGATCTTAGCCAGATATGTTTAAAATTTGAGCTTAGCTGCTATAATTAA
GAACGTACCCCTCGCAAGGGGAGCCTCAGAGACAGTCAGTATCTAGGCTTTACAGAAAGCCCACCTTCCAGCAGCGGCCCCGGCGT
GGCGCAACGAGACTACATTCCATTTTGATTATTAAATAAACTATAATAAAATGATATGGAGGAATCAACAAAGGAATGAATAATAA
TTTTGTAGTAATAGATTTTGAAACAGGAGGTTTAGATGCTAGTCTACACGATCCAGTGCAGATAGCAGCTATTATTTTAGACGGGA
AAACACTAAAAGAACGTGGAAGGTTTGTTTCTATGATAAAAGCCAGTAGAGACGTTTATGATACAGAGAATAATATAATTTCCCAT
AATATAGTACCTACAAGTCTTTTAACTAATATAGTAACAATGTCTACTAAGAGAAACAGAGAAAAATACGAGGTACTTTTAAAGGA
AGAAAATATTTCTAGGGATAGGATTGACTAGAAATAGTTTTTAATTAAATGCCCTCCTTTAGTGGAGGGCATTTTTTCTTAGTGAA
AGTACTTAGCAGTTTACCAAAAAAATCTATAAAAACTTTTAAAATATATATAAATTTTCATCAACAATGTGTCTATAATAAATTAA
ATGAGAGTAAATAAAGTTTTCCGTTACGAATTAAAACCTAACGAAGTACAAAAACAAATTATGGCGCAGCATGCCGGATCGGCTCG
TTATGCTTATAATTGGGCTCTGGAAAAAATAAACAGTAAAGTTTTTAATACTACAAATGATAAAGATCTTTCTTTAAAAGAAAGAT
CTTGGATCTGTAAATCTTGTAATACAGATCATGATCGGGATCTGAATGCTGCAATTAACTTAAAGAAAATTGGAGTACTCTCTCAT
TCGAGAGGAATTTACGCCTGCGGAGACAGCTATGCTGGATGATCGTCTTGCGTTAATACCGCAAGACCTAAAAAGGTATCTGTCGT
TGACTGAACAGCGAAGTTGTGAAGACTTGAATCAGGAATTAGAAAGAGATCTGTTAAATGTTTAGATTTTTACAGATTTTCAGGAA
CGGCTTTAAATTTATATATGAATAAGAAAAAATTTAATGATGATTTTGATGAAATGGTATATAACTTATCAAAAATATTTAAAATG
AAAGATTCTGTTATTCCTTTTTCAAAAATACAATCCAGGCTTTATCAGTTACTAAGAGAAGACCGTTATAATATTTTGACT (SEQ
ID NO: 366)
>3300007960|Ga0099850_1017825_5|M
CTCTGCTTCTTCCTCCATGCGTTCCCGAATGTCGTGTTCGTCCCATTCCGGCGAACCAACAAATGCGTCTAGGAGCGCCGCCAACG
TGTGGCCGGGCTGCCAAGAATCAATCTCGGCTTCTGTCGGGAAATCTGCGGGGCGTGTGTTCATGTTCTGCCTGCCTCTCTCCGCC
TGCCAGCGGGATTGTTGGTGAAATGGACGGTGACATGTCTCAAGCCGTCCGGTACTCGGTGCGCCTGCCGTGCACTTCGTACTGAG
GTCAAGTATACTGCAAAATTCTGCACTTGTCAAGCATTAATTTGCGTGAGTTTCTTGACAGTCTGCACAAGTTTGCAGTATAATGG
ACTCATGAACAGAGCACACCGTATTAGACTAAATCCAACAACGGAGCAAGAAGAATACTTTTGGCAGTGTGCGGGGGTGTCTCGGT
TTACGTGGAACTGGGCATTAGCAGCGTATAACGATGGCGTTCCGTTTAGCTACTTAAAAACAGAGTTCAATCGTTTGCGCAGTGAA
GACGGGTTTGCCCCGTTTGTATCAGAGGTTCAAAGCTACGCATATCAGCAGGCGTCAAAGACTTGTAACGCTTGCGGGGAAAAGGT
AGATGCATTGCCGTTGTCGATTCGCCAGTGGGCTTGCCCGAGCTGCGGCGCAGAACACGACCGAGACGGGAATGCTGCCAAGAACA
TTAGAGACGAAGCCTTGCGAATGCGAGGATGATTGTCTAGCCTGCGGGAGTACTCACATCGTGGGCTTAAAAATGGCCTGGGGAGG
ATAGCACGTAGGTGCGCCGATGAACCAGGAACCTACACGCACATTACAAACGATAAACGCACGTGGAACAAACATGCATCGGGGAG
TTCATCCGCAGCGGGCACATTGACGACTGCA (SEQ ID NO: 367)
>3300025687|Ga0208019_1031224_1|M
AGTAAGCCTTGTACGCTGCCGTGCCCGGCACGAAGTACTGTTCCGGCACGAAGTGAATGCCGTTGGCGCAATCCTCTGCGCCCATG
CGCCGGTACTCTGCTTCTTCCTCCATGCGTTCCCGAATGTCGTGTTCGTCCCATTCCGGCGAACCAACAAATGCGTCTAGGAGCGC
CGCCAACGTGTGGCCGGGCTGCCAAGAATCAATCTCGGCTTCTGTCGGGAAATCTGCGGGGCGTGTGTTCATGTTCTGCCTGCCTC
TCTCCGCCTGCCAGCGGGATTGTTGGTGAAATGGACGGTGACATGTCTCAAGCCGTCCGGTACTCGGTGCGCCTGCCGTGCACTTC
GTACTGAGGTCAAGTATACTGCAAAATTCTGCACTTGTCAAGCATTAATTTGCGTGAGTTTCTTGACAGTCTGCACAAGTTTGCAG
TATAATGGACTCATGAACAGAGCACACCGTATTAGACTAAATCCAACAACGGAGCAAGAAGAATACTTTTGGCAGTGTGCGGGGGT
GTCTCGGTTTACGTGGAACTGGGCATTAGCAGCGTATAACGATGGCGTTCCGTTTAGCTACTTAAAAACAGAGTTCAATCGTTTGC
GCAGTGAAGACGGGTTTGCCCCGTTTGTATCAGAGGTTCAAAGCTACGCATATCAGCAGGCGTCAAAGACTTGTAACGCTTGCGGG
GAAAAGGTAGATGCATTGCCGTTGTCGATTCGCCAGTGGGCTTGCCCGAGCTGCGGCGCAGAACACGACCGAGACGGGAATGCTGC
CAAGAACATTAGAGACGAAGCCTTGCGAATGCGAGGATGATTGTCTAGCCTGCGGGAGTACTCACATCGTGGGCTTAAAAATGGCC
TGGGGAGGATAGCACGTAGGTGCGCCGATGAACCAGGAACCTACACGCACATTACAAACGATAAACGCACGTGGAACAAACATGCA
TCGGGGAGTAAATCGGCTTGTCGTCTTT (SEQ ID NO: 368)
>3300009089|Ga0099828_10012481_2|M
ACCACGAGGTTAAGCCTGCATATGTCAGAGCGCTGCGAGAGGCCGGGCTGATGGATCTCTCTGTTGACCAGGTGATCGAATTGAGC
GAGCATGAGATCAAACCTTCCTATCTCGCCAAATTGCGCGAGGCTGGATTGACCGATCTTACTTTTGACCAGGTCATCGAATTGAA
CGATCACGAGGTTAAGCCTGCGTATATCAAGGCGCTACGCGAGGCCGGGCTCGTTGACCTGACCTTCGACCAAGTGATTGAATTGA
GCGAACGAGAGGTTGATCCCGCTTATGTCAGGGCACTGCACGGCGAGATTCATTGATTTTCTCGTGAGGCAGATCACCCGGCGATA
CAGGATCTGCACAAGATCCTGACGCTATTGGGGTGACCTGTCTTTTTTTATCAAACCGCGCCATAAAACCCCTCGCTTCAGCGAAG
GGGATACAAGGCGCGTTCCTCTTGGGGCGCCTTGGGCACGGGGCTCGTGCCATTTCCGACTTGCTCTCTTAGAAACAGAATGGTAG
CATATGAGCAGGAGCTACTGCGAGGAGCAGACTGTGACGAAGCGAGCATACAAGTTCAGGTGTTATCCGACAGATGAGCAAAAGCA
GATTCTTGCTCGCACGTTCGGCTGCTGTCGCTGGGTCTACAATTGGGCGTTGCGTCTGAAAACCGATGCGATCAACATTCTGAATG
AAGCATTGAGAACTACCGGGAGTTACTCGGGAAGTCACGCCTATGGAGAGAGCAGCTCTGGCCTGGTCAGTGGATCAGGTGAAACT
GCTCTCGTCGAAATAGGAACCAACCTGCATGTAGGCATGTCCTAGATGTCTAAGTATTGGAGAACGGATTTTTATGAGCAAAGGTG
AGCAAACGAAAGAGAGAATCCTCGGACGAGCCGCGCAGCTTTTCAACCAGCAGGGATACTTCGGCTCGTCACTCTCCGATATCATG
CACGAAACGGGTTTGGAGAAGGGCGGTATCTATAATCATTTT (SEQ ID NO: 369)
>3300006417_501592_1|M
TGGGCAGGTCGCCAGACGATGGCGACGCGGTGGTGATGGCGTTCTGGGAAGAACCGCCTGAGCCGACCGGCGGCGTGGGGTTTGTG
CTGTAATGTTAAAAGCGCATGTGATCCGTCTCAACCCAACCGAAGAACAGGCCAGCTACTTCTGGCGCTGCGCGGGGATTGCTCGC
TTCACCTGGAATTGGGCGCTGGCTGAGTTGAACGCCGCCTATGACCGGGGTGAACGGCCTGCGATTGGAACCTGCAATGAGTGCGG
CCACGTCATGGCCGACATGCCGCTATCGGTGCGCTGGTGGCAGTGCCCGACCTGCGGCGCAGAACATGATCGGGACGGCAACGCAG
CGGTCAACATCCGCAACGAGGCCGTGAAGATGGCAGGCGCTGCCTAGTGCGCCCGTCGCGGTAGTAATCACGTCGAGACGCAAAAC
GCCTGGGGACGGTGTAGGTAGCAAACCGGATGAACCAGAAATTCACGCAATTGAATAGGTGAACAAGCATAACACCTGGAAGGAGT
GTCTCAGCGACACAACTAAGGACTGACCTATGACGATCCTCATCACCATGCGCGAGCCGAACCGGGAGGACGCC (SEQ ID NO:
370)
>3300025018|Ga0210043_1027795_3|M
TCGCTCGGCAAGAGCCAACTCCAGAACGCGCACGATCTGCCAGTTTAGCGGGCGACGTTCCTCTTGCGCCCACCTTTTCAGCGCGG
CGTGAAGCTCGGGCGGCAAGCGCAACAAGATGTTGATGGTCTTGGGTTTCTCTTTCTTTGCCATATCACCAGTATATCACAGTGAT
GCAAGATGGTATCAACCCAGGAGTCTTTGATGCTGAAGTTCCATCCCGTTGCCGAAATGAAAATCCGTGAGGACACATGAATCGTG
CTCATCGCATCCGGCTCAACCCGACAGCCGAGCAGGCGGCCTACTTTTGGCGCTGTGCCGGAGTCGCGCGCTTCACCTGGAACTGG
GCCCTGGGCGAATATAATGCCGCCCTCGCCCGCGGCGAAAAGCCAAGCCTCACCCCCAGTAGTAAAACGTGCAACGACTGCGGTCA
GATCGTCGAGTTGACCCTGGCCGAGCGCACCTGGACTTGCCCCCGCTGCGGGGTGATTCATGATCGAGATCTGAATGCCGCCCGGA
ATCTGCGAGACTACAGTGCAGTGACCGGTTAGGAATGCGCCGCCGGAGTACTCACACGGAGGCGTTAAACGCCTGTGGATGGTGGT
CGTAGTAGAGGCGCCAGATGAAGCAGGAGTTCACACATACGTGGATAGAATACGCGTTAGGGAACGCACGGCGTACCTGGCAAACG
GGGCCTCGG (SEQ ID NO: 371)
>3300017963|Ga0180437_10001136_84|M
AGAAGGTCGCACGCACTGAAACTTGATCCGAATGCCGGGCAGGCGCGGAAACTGCGGCTGTACTGCATCGCGCATTGCAAGATATG
GAATCATTTTCTTGAAAAGTGCAAAAAGGGCTTGATTCCGAAAAAAAGTAACGCGATGACGCATCAGCTCGGAAATAAAAGGATGG
ATAAGACCTGTCCGGAACTGCTGTGGGTCCGGGATGAGCTGGGAATGAACAAGCAGATTCAGCAGGTTCCATGCGAAGATCTGGAG
AAGGCATGGAAGCGATTCTTCGACTGGTGCAGGGCGAGAAAGAAGGGGAATGTCGGTCTGCAGACCTGCTTTCACTGCAAGCACGT
GAACGATTTCGTGACGCTGAATATGAAAACGTTCACCTGTCGCGGATGCGGACGAAAGATCGAGCGGGAATCGAATGCTGCCGCCA
ATGTCAGGGAGATGGGATTCAGTGAGCTTGGTCTTTAACAGTATTGAATGTCGCCGTCGGGCAGACGGAGACTCTAAACGCTCGCT
GAGCGGACGGCCGCTGTCGTGGAAGGATGTGCTCTTTCGCGTTCATGCTGACTGCGTTGAAGCGAGAATTTCTCTACGATTCGAGA
GGTGCGGTGACCTCAGGGTTGCCGTACCCTCTCGAATTTTTAAACTGATTTCCTGCAATGATTTAGGGAAATCTCATGTTCGACGC
AGCGCATTATGTGCGTATATTTATGGATCGCTCGAAAAGCGATATACAAGTAACTGTCAGCAACATGCTTATGCATGGAACGGTGT
GAATGCGTGAAATTGAGTAGAGGATATGTCAGTAAGGGTCGACCGTCGGGCATGCCGGAGGTGGCGTGTGTGAATGCGTGAAATTG
AGTAGAGGATATGTCAGACTT (SEQ ID NO: 372)
>3300006417|Ga0069787_10357475_9|M
CATCGCCAGCTTACCGGGCGCTGCCCAGTGTGCGGCGAGCCGGCTTTTGGACCCGGTGTCACGTGCAAACGGCTGACTTGCATATC
AGATTGGGTAGGCACCAACAAGGAGCACATCAAGGAGCACATCAATGACGAACCGACTGATTGACGCAAGAATCTGAAGCTGTTTC
CAAATTTGACAACGTAGGTATTTGTAGGTAAAATAAATACACCATGAACGCAACAGTCAAACCACTGAGCATCCGTGTCCCTCCTG
ATATGCGAGAAAAGATCAATCGCCTGGCCCAAAGCGAGCGCCGTTCCCAACACTCGATGATGTTGATTCTGCTTGAAGACGCGCTT
CTAAAGCGGAGCGTAGTAGAACAAGCACCATCGAGTGAAGCGACCGCATCTCTTGTTCTGCCAACGTTTCGATCTGCTGGCGCATC
TCAACAGGCAGACGCACGCTAAACGGGCCGTACTCTTGTTTGGTTCGTGGTTTCGCCATGTAGTTATTCTACCACCAAAACAGGTA
CACGGTCAACATGCTAAAAGCTCACGTGATTCGCCTCAACCCAACCAAAGAACAGGAAACGTATTTCTGGCGCTGCGCGGGGGTTG
CCCGCTTCACTTGGAATTGGGCGCTGGCCGAGTTAAACGCAGCTTACGAGAAAGGCGAGCGGCCTGCCGTTGGAACATGCAACGAC
TGCGGCTACGTGATGGCTGACATGCTGCTCTCGGTGCGCTGGTGGCAGTGCCCGGCCTGCGGTGCAGAACATGATCGGGACGGCAA
CGCGGCGGTCAACATCCGCAACGAAGCCGTAAAGATGGCAGGCGCTGCCTAGTGCGCCCGTCGCGGTAGTAATCACGTCGAGACGC
AAAACGCCTGGGGACGGTGTAGGTAGCGAACCGGATGAACCAGGAATCTACGCACACTAACAGGTGAACAGCACAATACCCGGAAG
GAAGATAAAGATAAAGCGGCCGCGCCGCCAATTTACCCCACGGTAGCGTGGAATTATAGGATTTTTAGCTATGGCTATGGACTGGT
TTCGCTCCTGGCACAACGCACCAACAGACCCAAAATGGCTGCTGATTGCACGCAAGGCCAACGTGCCACCCGGCATGGTGTCAGCC
GTTTTCTGGGCGCTACTCGACTATGCAAGCCAAGAGAAGGAACGTGGCAGCATA (SEQ ID NO: 373)
>3300014060|Ga0119967_10019787_9|M
TCGCTCGGCAAGAGCCAACTCCAGAACGCGCACGATCTGCCAGTTTAGCGGGCGACGTTCCTCTTGCGCCCACCTTTTCAGCGCGG
CGTGAAGCTCGGGCGGCAAGCGCAACAAGATGTTGATGGTCTTGGGCCTCTCTTTCTTTGCCATATCACCAGTATATCACAGTGAT
GCAAGATGGTACTAACCCAGGAGTCTTTGATGCTGAAGTTCCATCCCGTTGCCGAAATGAAAATCCGTGAGGACACATGAATCGTG
CTCATCGCATCCGGCTCAACCCGACAGCCGAGCAGGCGGCCTACTTTTGGCGCTGTGCCGGAGTCGCGCGCTTCACCTGGAACTGG
GCCCTGGGCGAATATAATGCCGCCCTCGCCCGCGGCGAAAAGCCAAGCCTCACCCCCAGTAGTAAAACGTGCAACGACTGCGGTCA
GATCGTCGAGTTGACCCTGGCCGAGCGCACCTGGACTTGCCCCCGCTGCGGGGTGATTCATGATCGGGACCTGAATGCCGCCCGGA
ATCTGCGAGACTACAGTGCAGTGACCGGTTAGGAATGCGCCGCCGGAGTACTCACACGGAGGCGTTAAACGCCTGTGGATGGTGGT
CGTAGTAGAGGCGCCAGATGAAGCAGAAATTCAGACCAGATGTGTGATCCATCAACAAGCCATGTCGAGAGCCTGCTGACCACCTT
TGAGCGCCTTGTGCGCGCTCAGTGGGCGACTACCGGCCGCGGCTCGGAGCATACTATCCGGGCCTACCTGACCGATACTCGCCAAC
ACCTGGCCTGGCTGGAA (SEQ ID NO: 374)
>3300017971|Ga0180438_10021976_11|M
CAGACCTGCTTTCACTGCAAGCACGTGAACGATTTCGTGACGCTGAATATGAAAACGTTCACCTGTCGCGGATGCGGACGAAAGAT
CGAGCGGGAATCGAATGCTGCCGCCAATGTCAGGGAGATGGGATTCAGTGAGCTTGGTCTTTAACAGTATTGAATGTCGCCGTCGG
GCAGACGGAGACTCTAAACGCTCGCTGAGCGGACGGCCGCTGTCGTGGAAGGATGTGCTCTTTCGCGTTCATGCTGACTGCGTTGA
AGCGAGAATTTCTCTACGATTCGAGAGGTGCGGTGACCTCAGGGTTGCCGTACCCTCTCGAATTTTTAAACTGATTTCCTGCAATG
ATTTAGGGAAATCTCATGTTCGACGCAGCGCATTATGTGCGTATATTTATGGATCGCTCGAAAAGCGATATACAAGTAACTGTCAG
CAACATGCTTATGCATGGAACGGGTGTCCAGCCGTTCAGCCAGACGACGACGC (SEQ ID NO: 375)
>3300020180|Ga0163155_10006838_6|M
CATCTCCGATGCGACGCGTTGCATCGCTTGCTCCAACGTCACCAGGCTCTGTGCCGTCAGGATCTTTCGGCGAAACTTCGTTACGA
AGACCAAGTGCACTAGGAGCTTGGTCACGCTGTGACGACCGCGATCGAGCGCTTGCATGGCCATGTTTTGCAGAGTATGGTCAGTT
GCAGACCAATGGCAACCGTTCGCTACCGATATCGACTTTACCCTACGCCTGCGCAGCGCGTGGCGTTGGCGAGGACGTTCGGTGCG
GTGCGGTTCGTGTGGAATCAAGAGCTTGCGCGATCGCAGATCCATGATGCTAAATACCAGGGCTTCAGTGCTAATGCTGCGAAGAA
CATTGTCGCGGCCGGGCAGGTCGAGACGAAAAACGGACGTGGAGGGCATGTGAGACGGATGGTGCCTTTGGGTATCTGCGCAGTGC
TCGATGAAGCGTCAACCTCGGGGCCGACCCCGGAATCCTCAGCCTTCTAGGCCGAGGAGGATGTCAAGGCATAGGTAAACCCTAAC
ATCGGGGCCCCTAACATCGGGGCCGTACGAGAAGATCTGTTCGTACCCGAATGCCCTTGCGGCATCGATCACGACTGTAGAGATTC
TCTTGCGCAGGGCGGTCAACCCTTTCAGGGACGATGTCGGAGATGTTTTTTTCAGTCGCACGTGTCGTTACCTCTCGGGTGGCAAC
CAGATCTTTCCTGGTTCAGCATCAGGCATCCGCCTTCGCCGCGCGCTTGGTGGCGGTGGGAAACGCAATGGGGAACAGCGCGGCGC
ACTGGCGCGCGTACTTGCGGGTGATGATCCACTGGCTCGCGCTGGTCGGCATCGGGGTTTCGCTCGGGCCGGCTCCTCGCCAGGCT
CTCACACGTACGACGCCCCGCCGCGCGCGTCCATTTCCCTCCTGGTGCGCTCAGGCGGTCAGGCAGCGCACGAGGAGGTGCTGTGG
AGCCCAGGAAGAGAGGCACCAGCGCACGAGGAGGTGCTGTGGAGCCCAGGAAGAGAGGCACCAGCGCACGAGGAGGTGCTGGTAGC
CATCGGGTAGGTCGAAAATATAAAAAACATCATCCAAGCGCATCAGGAGCCGCATAGCGCGACTTCTGATGCGCTTGCACCCCCGG
GGGTACTCGCTTGTACATGGTGGAGTATTCAGACGATGACAGCTGCACGCACCGACACCGACGACGACCTGATTGCACGGGCTGAG
GTCGCGAAGATCTTCGCCGTCGCCGAGGGCACCGTTTCCGCTTGGATGGTCCGCAAGGGTCCTCAGATCCCACCGCCCGTGATCCG
CGGCAAGAACTTC (SEQ ID NO: 376)
>3300028299|Ga0268276_1017374_4|M
ATGGAAAAGAAATGCAAAACTACTATTGTATTTTCTGCTGTCGCCAAAGATCTCCTGGTCGCGCTGGCTAAAAAGTATGGCCTGAC
TATGACGGCCACGCTGGAGATGATCCTTCGTGACAGGGCTAAACAGGAGGGTCCCTGGCAATAATCCGCACCCATTGTGCATCTAC
TGAAAAATACATAACATCGCCGAAATACTCATGCGGAGATGTAAAACGCCTGGGGATGGTCGAGGTTCAACTAAGCCAAATGAACC
AGGAATCCTACACTCGTTAGGTAATGAAAATGCACAGAGCACATCAAATTCGATTGAATCCAACGCCGGAACAGGAACAATATCTT
TGGCGTTGCGCTGGCGTGGCCCGCTTCACCTGGAATTGGGCGCTGGCGGCTTACAATGACACTCTGGCCCGCGGCGAGAAACCATC
AAGTAGCAAAACGTGCAACGACTGCGGTTATATCACCACGCTAACGTTGTCTGACCGCGTATGGACATGCCCGAATTGTGGCGTTG
TCCATGATCGAGATATCAATGCTGCGCAAAACCTGCGTGATTATCAGCCCGCTAGCGCAGCTTAATGGAACACGTCGCCGAGGTAC
TCACGCGGAGACGCTAAACGCCTAGGGATGGTAGTGGTAGTAGTCACGCCAAACGAACTAGGAGTTCACACACTCGTTAGGGTTCA
CCCCGCGAGTGCGTGGAGTGAGGGTGCGATAGAATACAGATAATGAAAGTTGAGTAATATTGATAGCTGATTCTGGGGCCTAACTC
GTGATCTATGCTACTGTTTGGTTACCTAAATCACCAAGTGGGATTGCCCGACTACTAAAAGCCGGTTATCCGTGTGCTCCGTTTGT
GTTTACGGAACAAGGTCGCCAACTAGCACTTGGTATCAAACGTCCGAATCCCTACATGGCCCAAGACACATTGCCAGATGGTCGCA
TCATTATCGTAGTAAGTGCTATCGCGATCAATGGAGAGGTACATGATAGATTGATACCCGACCCCATTGCGCTACCGATACCCATA
CTAAGTTGGATGCTAACCGCTAGTAGCGGCGGTACACGTCTAACAGTGGTATTTGATCTTCCCGCGGTTTGGCAGTACGTTGATAC
TTACCTGGCGTTTTTAGCCAGGGATTTCCATGACCAGTTCCACGCAATCCAAACTGCCCGCGCAGCATTGGTGAATGCTGATTGAT
TTATGAGGTTTCACATGAGCGAACGTAGAAATATTTCCGTACTGGTCGAAACCCGCAAAAAGGTCAGGCTCCTGGCCGCTACGCTG
GGAGTCAACATGGCCGAGGCCGTCGAACTAGCAGTTGCTAAACTGCTGGACGAGAGCACGCCCGGT (SEQ ID NO: 377)
>SRR6837571_682758_2|M
CGGCTTTGCTATGGCGTGCGTCAACTGGCGGCCCGGCGATACGCCCGCGCCCTACCAGGTCGAAGTGCTGGCGGCGCTGGCCGAGC
GGCGGCGGACCAGTGTGCGCGGGCCGCACGGGCTGGGCAAGACGGCGCTGGCCTCGCTGGCCATCCTGTGGTTTGCGCTGACCAGA
GACGGGGAAGACTGGAAGATACCGACCACCGCCAGCGCCTGGCGGCAATTGACCAAGTTCCTGTGGCCGGAGATCCACAAATGGGC
GGGCCGGCTGCGCTGGGAGTTGATCGGCCGCGACCCGCTGAACCGGCGCACGGAGCTGCTGACGCTCTCGATGAAGCTGCGCACAG
GGGAGGCATTTGCCCTGGCCTCGGACAATGCGGCGCTGATCGAAGGCGCGCACGCCGATCACCTGCTGTACCTGTTCGACGAAGCG
AAGGTTATCCCGCCGGCCACCTGGGACAGCGCAGAGGGCGCCATGTCTTCCGGTGACACCTACTGGCTGGCCATCTCGACGCCGGG
CGAGCCGGTGGGGCGCTTCTACGACATCCACCGGCGCCGGCCAGGCTATGAAGACTGGTGGGTGCGCCACGTGACCAAGACCGAAG
CCATCGCCGCCGGCCGCATCCGGGCCGATTGGGCCGAGAAGCGGCGCCAGCAGTGGGGCGAAACATCTGCCGTCTACCAAAACCGT
GTCGAGGGGCAATTCGCCAGCGGCGATGAAGACGGCGTTATCCCGCTGTCGTGGATCGAGGCGGCTAACGAGCGCTGGCACAGCCT
GAACGAGACGGACGGTTGGGGGCCGCTGCTGGCCGTGGGCGTGGACGTGGCGCGCTACGGGGAAGACCGCACGGTGCTGGCGCTCA
GGCACAGGCCGGGCGTCAAGGCGCTGCGCCGGCACGCCAAAGAGGATACGATGGCTACATCGGGCCGGGTGACGGGCATCCTGGAG
GCGCAAGGGGGCCGGGCCGTGGTGGACGTGATCGGCGTGGGTTCGGGCGTGGTCGACCGGCTGCGCGAGTTGGGCAAGCCGGTGCA
GGCGTTCAACTCAGCCGAGCGCACGGACAAGCGGGACGCCTCTGGCGAGCTGGGCTTTGCCGACAAGCGCGCGGCGGCCTACTGGC
AGGTGCGGGAGATGCTGGACCCGGCCAACGCGGACGCCATCGCCCTGCCGCCGGACGATCTGCTGACGGGCGACCTGACCGCGCCG
CACTGGCGGGTGCAGTCGGGCGGCAAAATCCGGGTGGAAAGCAAGGACGACATCCGGGCGCGGCTGGGCAGGTCGCCAGACGATGG
CGACGCGGTGGTGATGGCGTTCTGGGAAGAACCGCCTGAGCCGACCGGCGGCGTGGGGTTTGTGCTGTAATGTTAAAAGCGCATGT
GATCCGTCTCAACCCAACCGAAGAACAGGCCAGCTACTTCTGGCGCTGCGCGGGGATTGCTCGCTTCACCTGGAATTGGGCGCTGG
CTGAGTTGAACGCCGCCTATGACCGGGGTGAACGGCCTGCGATTGGAACCTGCAATGAGTGCGGCCACGTCATGGCCGACATGCCG
CTATCGGTGCGCTGGTGGCAGTGCCCGACCTGCGGCGCAGAACATGATCGGGACGGCAACGCAGCGGTCAACATCCGCAACGAGGC
CGTGAAGATGGCAGGCGCTGCCTAGTGCGCCCGTCGCGGTAGTAATCACGTCGAGACGCAAAACGCCTGGGGACGGTGTAGGTAGC
AAACCGGATGAACCAGGAGTTCACGCAATTGAATAGGTGAACAAGCATAACACCTGGAAGGAGTGTCTCAGCGACACAACTAAGGA
CTGACCTATGACGATCCTCATCACCATGCGCGAGC (SEQ ID NO: 378)
>3300020219|Ga0163146_10008638_12|M
GCTCTTGGCGTCGCTCCCCTCGAAGTCGGGCAGGTAGTCGTTGACGTTCTTCGCCACCACGTCAAACACTTCGCGTTCGGTGCGTT
CGATGATGCTTCTCGGCTCGCCCTCGAAGGGGTAGACCCGCTCCGCCTTCCACTGCTCGATCAACCCCCGAGCTTCCTGCACGCGA
CGTGCGCGGAAGTATAGTCTTGCGCAGACCAATGGCAAGCCTCCGCTACTCGTACCGCCTCTACCCGACGACGGAGCAGCGCCGCG
CGCTTGCTCAGACGTTCGGGTGTGTGCGTGTCGCGTGGAACGACGCTCTTGCTCAGTCTCAAGTGAGCGGTGCGAAGTACCCTGGG
TTCGCGGCGACGAGCAGACTCCTTACGGAGTCGAAGAAGTCGCCTGAGCGCGCGTGGTTGAACGACGTGTCCAGCGTCCCGGTGCA
GCAGTCGATCCGAAATCTCGACGTGGCCTTCCGCCGCTTCTTTAACGGGCTGAAAGGCAAGGGGCCGAAGGTCGGCTTTCCAACTT
GGAAGCGGAAGGACGGCAAGCAGTCCGCCGAGTTCAACGCCGCTAAGAACGTCGTGGCGGCAGGGCTTGCCGACACGGAAAACGGA
CGTGGAGAATGGGTAAGCCGGGTGGTGTCGATGGGCACTGGCGTTCCGTTCGTTGAAGCGTCAACCGCCGGGGCCTCCCCCGGAAT
CTCCGCCCTTTAGGGCGGGGAGGATGTCAACGGTACGGCTCGCCGGGAAAGATAGCCCAGGGCGTTTGTGGGGGTTCTCTAAAAAC
GGAAGTGTGTGAAAAAAATGGGCGCGTTTGCAGAGGGACGTCGCGCTGGTCCCCAACCCCCCTTGCCATGGGGCTTTTCTCATTCT
GCGAATCGCCCTTGCCATCGGCCCTTGGGCGCATGGCAAGGGCCCTGGGACGAGGGGGCCCTGGGACGAGGGGGCCCTGGGACGAG
GGGGCCCTGGGACGAGGGGGCCCTGGGACGAGGGGCCCTGGGGCGAGGGGCCCTGGGA (SEQ ID NO: 379)
>3300001380|JGI1356J14229_10025169_2|P
TTGGGGGGTTTGGAGTAGTTGTCCGGAGTCATGATGTTGAGGTTTTGCCGCGTGCCAAGGTCTATGCCAACCCTGTCCCCGATTTG
GCACCTGGCGCCAAGGTCAAGCGAATGCCCCCACACGCCCGTCTTGTCGCCAATGGACTCCACAAGGTATTTTGCAAACGGCCCGA
CCGACTGCTTGCTGCCCATGGAGAGCATGTAATACGCGCCAGCCGCACCTTCCACCTGGTCCAGCATCTTGAAGCTAAGCTGCACT
CCGGCATATTCGTTCTGGTAAAACGCATGGGCCTTGGGGCCCGCATACACTTTGGGGTCGATGCTCCTTCCCGCCCCGGTATTTTT
CATCGTGCTTGAAAGGTAGCCTGCACCGCCAAGGCCAACCCCTATCCGCCCCGCACCCGGCTTCCACACCCAGCCAAGGTTCAAAT
CCACCCCTACCGTGCCCATGAAGGCCTTGTCGGTTGGCGTCACCCCGCCTGGCAAATACATCTGCATCATCACTTTGGGCGAGTTG
CCATCGAACCCAATCTTGGAAAGGGCAAAATTTTTCCTGTTCACATTTCCAACCGCAAGCCCCATCGCGGAAATATTGAACCCTTC
CTCCGGCTTGTCCTGGTAGCCGAATGCAAATCCAAGGTATGGCACTTCGGAATCAAGCTGGGTCCTTTTTCCCCACGCGCCAATGA
GGTTCAGCGGGCTTTTCTCCACCCAGGCGGCAACGCCAAGGCCGTGCCCTAAATTGTACCCATAATATATCCCTGCCTGTGCGGCA
TCAGTCACTTTTGTCGCAACAGAAAATCCCATGTTGGAAAATGCGAAATTGGCCGACTCCTGCGCCATGGAAAACAGGCGCCCGGT
CCCCCGCTTCAAAAGCTCGGAATAGCTTATGTCAAGAAGCGCATTGGCCGCAGCTTCCAACCGGAGCCTGCCCGTTGTTTGGGCAT
CCGGCCCCCTTATTTTCTCAATTTCAACAAATTCGCTTTCCATCCCGTTGGCATAAAACGCCTGCTTCAGCGATGGAAGCGAGAGC
AGGCTGCCGGCAACGAGCTTCTGCGCCTTGTTGGATTTCCAATCTACATTTTGCACTTGCGGGAAGATTGTTCCCAAATCCATGGC
GCGGCTGCGTGTTGAGGAAAATGCGTTCTCCCCAGGTTTAGCCTCCATCGGAATTGCGCCATCCACCAGCCCGTCATCCTCCGGAA
CCGTGGTTGGCTTCTTTTTCCCAGCATCAGCAAGGTCATCCTCCGGAATCGTGGTCGGGATTTTCTTCTTTTTTGCCTCTTCCTCT
TCAGGCGGAACTGTGGAAGGCTTTTTCTTTCCTCCATCCACCAGCCCGTCATCCTCAGGAACAGTGGTTGGCTTCTTTTTCCCAGC
ATCAGCAAGGTCATCCTCCGGCACAGTGGTTGGCTTCTTCTTCCCAATATCAGCAAGCCCATCCTCCGGCACAGTGGTTGGCTTCT
TCTTCCCAATATCAGCAAGCCCATCCTCCGGAACCGTGGTCGGGATTTTTTTCTTTTTTGCCTCTTCCTCTTCAGGCGGAACTGTG
GAAGGCCTCTTCTTGGCGGCATCCGCCAAATCATCGCCAGGCACTGTGGATGGCGTTTTTTTCTTTCCAGCGTCTGCCACTACTGG
CGGCTGCTCGGCTTCGGGTTTTTCAGGCGCATCCTTTAGGGGAAGAGCAAGGCCGTGCGCTGCCATTCTCCTGTTCCATGCTTCAC
GCACCGCCAATGGCACGCGTTCACTCACGGCTTCGGCCGGATTTTTGCTATTTCGAATGGCCGCATCCGCCAACTTGATTATCATG
TCTTCTTTTTCCTTAGCAGTGCCCGCGCCTTTCATTTTCAGCAAATCCTGGGCGAATTTCCGAAACGAATCCCCCACCGCGATGCT
TTTGTTGCGCTCGGCAGGAACGCGCCTCCAATAGTCATCCTCGGTTATTTCCCCGGCGTTCACTTTCTTCGTGTTTCCATTCATGA
GGCCGTCATATTTTGCCCGTTCGATTTCCTTTGCCTTCTTCTCCGCCTGCCCAAGCTGCGCAGTTTCCCCGTATTTCACCGCCTGC
ATCAGGAAAAACATATTTTCATCCGACAACTGCTTTCAAATGGGCGATTTATTAATATGCATGGTGCGCGCTAAATCCAGCGAGTT
TTACATGGCACTCCGCCGCCTTAACTCCGGAGTTTTCGGAAAGCCCGGAAAGCCACCCTTTCGAAAGCCGTATATAAACCACATCA
TTCTTTTTTCCGGGATGTTCCAATGAGGTCATACAAATTCCGCATCTATCCTTCCAAGAATCAGGAAAAGGAAATGCTGCATCACT
TATGGGTTGCAAAAAACCTATGGAATGAATTACTTGCACACTGCAAATCATTCTATGCCTATTTCCAGAAATTCCCCTCAAAATCA
GCGCTGCAAATCATGTCAAAGAGTTCAGGTTTGTTTTCACAAACCTCACAGGAAATAGCGCACAGGGTGGAAAAGGCAATTTGGAG
ATTTGTCATGATGAAAAAGCAGGGTAATAAAGCAGGCTTCCCAAGGTTCAAGCCTTTTGAGAAAATGAAATCATTGCATTATCCGC
AATATGGAAACGGCTTTTTCCTTGATAAAACGCTTGAAGCCTCTCCATTTGGGAAAATCCAGATTGTAAGGCACTCGTGCGGCAAT
TCCCTTGACCGGGATATAAATGCCGCCCTGAACATACTCACACGAGCTACGGCCGGAATGGCCGGAAGTAACGCCTCCGGAGACGA
AACGATAATTCCGTCATTGAAGGAGGAAGCCCACGCCTTCACGCGTGGGTGATTCACCTTATTCGAGCGGCTTGCGCGCAAAAGCA
AGATACCCGGTGTGCGTGAGGCCGGTGTTTTCCGGGCGCACCCCCGCATCGCGCACCAGAATCTCGCGCATAATCACTTCGCAGCA
AAACGTGCCAGGGGAAAAGCC (SEQ ID NO: 380)
>3300022602|Ga0248169_134333_4|M
TAAAAGATGTAAAACACACTGTCAAAACACAGTTTGAAATTAATGTAATATACGAATTTTCATCTAGCAAATAACAATGGCTGTAC
ACCTACTTAATTCGCCAATCATGATGGACATGGGGACCTTCATACATGAGGGCCCTATTCGCCTTCCGGAAGTAGAAAGAATCATA
AAAAATGGCTTCATTTCTGGAATTACCCAGCTTGATACAGCCAGATACTTAACATCACTGTTGGGGGTTCATGTGGAATTTAAACC
AGTTATTACATTGCTCTTCAAAAATCACCCTTAACCTCTTAAGCATTTCTTTATTGATACATTGAGAGCGATATTTGGTTACTAAC
ACTAAATGATAATTTAGCTTATAAACAGCATGGAAGTGGCTTTTTGTAAGGGTATTTTTCATATTCACATATTAACACTAATTGAT
TTATCTGTTATAATAGATCCATGCTTTCGCACAAAGGATATGTTTACCGATTAAAGCCTAACGCTGAACAAGAACTCTTACTTGCC
CAGCATTTTGGCCATGTCCGCTTTGTTAAGAATTGACAAGATCTATAGCAAAAACTTGTCTCGCCGCCAGCTTCAAGATCACCTAG
TTAAAAAGAAGAAAAAGAGCAAATTTACATGGCTCAACGAGGTGAACTCTCAATCATTATTAGCAGCTCTATTGGACGTAGATAAT
GCTTTTAAGCGATTTTTTACACAAGGGGCTGGTCAATACACCAGGTCGCTAGGTACTTGCGAAACTGTAAAGCGCTCCCCAGTCTC
TATATCCGTTGGTGCGGATGAGTTTGCGAAAGGTTCCAAGAAGTTTGGAACGGGTCGGTTAGAAGAAGCTCCAGCTATAGCCGCTT
TAGCGGCTTAGCGGGAGTAGTTCACCTTCACCAGGTTGACGATGATTTCCAAATGAAGGCACCTTCAAAAGTCAGCCACTAATCTT
ATCCTCAATTTGCTTAATCACGCTTCTACCAGAATCATCTTTCACAGCAAATAGAAACAAGCATTTCCCTCTACTGGCTTTTTCCC
ATTGCAAGCCAATATTTTTCTTCTCTTGAGAATCATCATTGCTTGCATAAACCTCGCCCTTGTATTCAATCGCCATTATTCGGCCA
TCAGTAAGCTCAACAA (SEQ ID NO: 381)
>SRR1221442_902393_36|P
CCAAAGGTTGTAAAAGTTACAAAAACTGATTATACTCTTGACAATGATGATGTTTATGAACATACCTTTAAAAATAGAATAACAAT
TTATGTTAAGAGCAATCAAAATAAGATTATATCCAAACAAAACACAAGAACAAACACTAAATAAGGTGCTTGGTAGTTATCGTTTT
GTCTATAATCAAGTACTTGCTCAAAAACAAAATGCTTATAAACAAGACAAGACAAACCTAAAGGTAACTGATTTGTCAAAGTGGTT
TCACTGAACTCTGCTGAAGGATGAACATTATGCTTGGTTGAAAGAACAGAACACAAAGGTGATGAAACAAGCCATCAGACAGATGG
ACGGAGCGTACCAGAAGTTCTTCAAACAACATAACGGATTCCCAAAATTTAAATCCAAAAAGGATAAGCAATCCGCATTGTTCCCT
TATGAAGCTATTTCAAAACATAATACATTTGAAACAAGAACTTGTTCGGTTTGTGGTTACAAGAAACAAGATTTGAGGTTAAGTGA
CAGAGAATGGGTTTGTCCTAAATGTGGAACAAAACACAATAGAGATATAAATGCTGCTGTGAACATATTGTTGGAAGGACAACGAA
TGCTCACAGCAGGATAAGATAAAATGAAATACAGTAGGTGTCCGTAGCACCGAATTTACGCTTGTGGACTATCCTCCTATGGATGA
CCGTTCTGTAATGAACCTAAAAGTAGTGATAGATTGAAACAAGAAGTGAAATATACCTAAATCATAGATTTTCGTAGAATTTCATA
TACGGTAATTTCAATGATAACAGACTTACTTCTGATATGACAGAAGATGAATGTTATCTCTTGGTTACAGGTAAAACCAAGGCACA
ATACGAAGAAAGTAAAAGAAAAAGTGAAGAAGAATATCGAAGAAGAGAAGAAGAGGATAAGAAACAAATTCCAGAATTATGTAAGG
CATATGAGAAGGATTGGAGTTTCTACAGTCTTCTGTGTCTTGGTATTTGCAACTGCACCAAGATTTCTTTGAATAGGACGTCTAAC
TCTGTTTAAAGTTACACATGGTAATACTACTTTTTTCATATTATTTTTTAATTTATTTTATTGAATATTTGTCAAATTAGTGAAAC
TGCACGTTGATACATTACTGTTTCAATATATGTATCAATATCTCTTTTTTCAATGCCTATTGATACAAGTATGTCTGTCATCTGTT
CAACATCTTCTGAGTCAACACAAAGGACAAACCAACCTTTCTTGTCACT (SEQ ID NO: 382)
>3300025586|Ga0207996_1000986_11|P
GCGAATGACGATGTGAACGTGGTCGGCCTCGCCATTCAACTCAACGACCGTGAACGCCATCTCCGTCGCGACACGGCGCATCGCCG
CCTCGATCGTTGCGAGCCCTTTCGCGTCCAGGACCTTCCGCCGGTACTTCGTTACGCAGACCAAGTGGGCCAAAAACTTCGTAACG
CTGTGCCGCCCCTTGTCTAGCCCTTGCGTTTTCGCCACGCGGGCACCATACTCTCGTGCAGACCAATGACAAATCGTCTCCGCTAC
TCGTACCGCCTCTACCCCACGCCGGAGCAGCGCCATGCGCTCGCTCAGACGTTCGGCTGCGCGCGAGTGGTGTGGAACGACGCTCT
CGCCCGGGCTCAGGTGGGCGGTGCGAAGTACCCCGGTTTCGCCAATGCTGCCAAGAATGTTGTCGCGGCGGGGCTCGCCGAGACGA
AAAACGGACGTGGAGAATGGGTAAATCGGGTGGTGTCGATGGGCACTGGCGTTCCGTTCGTTGAAGCGTCAACCGCCGGGGCCTCC
CCCGGAGTCTCCGCCCTTTAGGGCGGGGAGGATGTCAACATCGACGCAAGACAAGACAGCTCTCTGCCGAGTCTAGCTGGCTTGAT
AGGGGAGGCTCTCGTCCTACGGTTACACACCCTTCTCCCTTTTTCGTGGCTATCTCCGATGCTCTTTCGGCCAAGATCTCCGAGGC
CCTGGAACTGATCGACTCCTTTGCGGAGCCCAAGCGCTCCGCCTGTCTTTGGAGCAGCGGCAAGGACTCCATGCTGCTGCTGCATT
TGCTCAGAAAGGCGGGGATCGACCTGAGGACTCTCTTTCTTCAGCGTCAGTCTACGAGGACTGAATGGCTTCCTCCTTAGCCGAAG
GCTTACGGATGTGGGAGCGAACGAAGCCAGCGCAAGCCGAAGGCTTACGGATGCGGCTGCTTAAGACGCTGCGCGTCTTTTCAACC
CTTCATACACCCTCTGCTGCCACCTCTTTTGATCCATTGCCAGCAGAGCGCAGGACGTACGCCGCTCTCGCATGTATCGATCCAGG
TGGCTGTCATCGTTTCGCAAGCCGAAGGCTTGCGGGGGTGAGAGCGATAGACCCTGCGCAAGCCGAAGGCTTACGGATGCGGGAGC
GAGAGACGCTGCGCAAGCCGAAGGCTTACGGATGCGGCTGCTTAAGACGCTGCGCGTCTTTTCACCCCACTGAGATGGGTGGCACA
TTCCTCCCGCATCCGATTGCTTGAGCACTGCAATGCGTTCTGCACCCACTTGTGGTTGACTCCCCTTGAAGCGCTCACTCCCCCTC
CTCCCGATCGTCTTCGCCCCAGCATTCCTTGCTTCCGTCGCGAAATCCCTCCAGGAAGCAGAATCCCTGCCACTCAGCCTCCCACC
TCTCGTCCAT (SEQ ID NO: 383)

### Example 8—Identification of Transactivating RNA Elements

In addition to an effector protein and a crRNA, some CRISPR systems described herein may also include an additional small RNA that activates robust enzymatic activity referred to as a transactivating RNA (tracrRNA). Such tracrRNAs typically include a complementary region that hybridizes to the crRNA. The crRNA-tracrRNA hybrid forms a complex with an effector resulting in the activation of programmable enzymatic activity.
tracrRNA sequences can be identified by searching genomic sequences flanking CRISPR arrays for short sequence motifs that are homologous to the direct repeat portion of the crRNA. Search methods include exact or degenerate sequence matching for the complete direct repeat (DR) or DR subsequences. For example, a DR of length n nucleotides can be decomposed into a set of overlapping 6-10 nt kmers. These kmers can be aligned to sequences flanking a CRISPR locus, and regions of homology with 1 or more kmer alignments can be identified as DR homology regions for experimental validation as tracrRNAs. Alternatively, RNA cofold free energy can be calculated for the complete DR or DR subsequences and short kmer sequences from the genomic sequence flanking the elements of a CRISPR system. Flanking sequence elements with low minimum free energy structures can be identified as DR homology regions for experimental validation as tracrRNAs.
tracrRNA elements frequently occur within close proximity to CRISPR associated genes or a CRISPR array. As an alternative to searching for DR homology regions to identify tracrRNA elements, non-coding sequences flanking CRISPR effectors or the CRISPR array can be isolated by cloning or gene synthesis for direct experimental validation of tracrRNAs.
Experimental validation of tracrRNA elements can be performed using small RNA sequencing of the host organism for a CRISPR system or synthetic sequences expressed heterologously in non-native species. Alignment of small RNA sequences from the originating genomic locus can be used to identify expressed RNA products containing DR homology regions and sterotyped processing typical of complete tracrRNA elements.
Complete tracrRNA candidates identified by RNA sequencing can be validated in vitro or in vivo by expressing the crRNA and effector in combination with or without the tracrRNA candidate and monitoring the activation of effector enzymatic activity.
In engineered constructs, the expression of tracrRNAs can be driven by promoters including, but not limited to U6, U1, and H1 promoters for expression in mammalian cells or J23119 promoter for expression in bacteria.
In some instances, a tracrRNA can be fused with a crRNA and expressed as a single RNA guide.

### Example 9—Identification of Novel RNA Modulators of Enzymatic Activity

In addition to the effector protein and the crRNA, some CRISPR systems described herein may also include an additional small RNA to activate or modulate the effector activity, referred to herein as an RNA modulator.
RNA modulators are expected to occur within close proximity to CRISPR-associated genes or a CRISPR array. To identify and validate RNA modulators, non-coding sequences flanking CRISPR effectors or the CRISPR array can be isolated by cloning or gene synthesis for direct experimental validation.
Experimental validation of RNA modulators can be performed using small RNA sequencing of the host organism for a CRISPR system or synthetic sequences expressed heterologously in non-native species. Alignment of small RNA sequences to the originating genomic locus can be used to identify expressed RNA products containing DR homology regions and sterotyped processing.
Candidate RNA modulators identified by RNA sequencing can be validated in vitro or in vivo by expressing a crRNA and an effector in combination with or without the candidate RNA modulator and monitoring alterations in effector enzymatic activity.
In engineered constructs, RNA modulators can be driven by promoters including U6, U1, and H1 promoters for expression in mammalian cells, or J23119 promoter for expression in bacteria.
In some instances, the RNA modulators can be artificially fused with either a crRNA, a tracrRNA, or both and expressed as a single RNA element.

### Example 10—Functional Validation of Engineered CLUST.342201 CRISPR-Cas System

Having identified components of CLUST.342201 CRISPR-Cas systems, a locus from the metagenomic source designated 3300006417 (SEQ ID NO: 301) was selected for functional validation.

### DNA Synthesis and Effector Library Cloning

To test the activity of the exemplary CLUST.342201 CRISPR-Cas system, the system was designed and synthesized using a pET28a(+) vector. Briefly, an E. coli codon-optimized nucleic acid sequence encoding the CLUST.342201 3300006417 effector (SEQ ID NO: 301 shown in TABLE 17) was synthesized (Genscript) and cloned into a custom expression system derived from pET-28a(+) (EMD-Millipore). The vectors included the nucleic acid encoding CLUST.342201 effectors under the control of a lac promoter and an E. coli ribosome binding sequence. The vector also included an acceptor site for a CRISPR array library driven by a J23119 promoter following the open reading frame for the CLUST.342201 effector. The non-coding sequence used for the CLUST.342201 3300006417 effector (SEQ ID NO: 301) is set forth in SEQ ID NO: 373, as shown in TABLE 19. A separate condition was tested, wherein the CLUST.342201 3300006417 effector (SEQ ID NO: 301) was individually cloned into pET28a(+) without the non-coding sequence. See FIG. 1A.

An oligonucleotide library synthesis (OLS) pool containing “repeat-spacer-repeat” sequences was computationally designed, where “repeat” represents the consensus direct repeat sequence found in the CRISPR array associated with the effector, and “spacer” represents sequences tiling the pACYC184 plasmid or E. coli essential genes. In particular, the repeat sequence used for the CLUST.342201 3300006417 effector (SEQ ID NO: 301) is set forth in SEQ ID NO: 342, as shown in TABLE 18. The spacer length was determined by the mode of the spacer lengths found in the endogenous CRISPR array. The repeat-spacer-repeat sequence was appended with restriction sites enabling the bi-directional cloning of the fragment into the aforementioned CRISPR array library acceptor site, as well as unique PCR priming sites to enable specific amplification of a specific repeat-spacer-repeat library from a larger pool.

Next, the repeat-spacer-repeat library was cloned into the plasmid using the Golden Gate assembly method. Briefly, each repeat-spacer-repeat was first amplified from the OLS pool (Agilent Genomics) using unique PCR primers and pre-linearized the plasmid backbone using BsaI to reduce potential background. Both DNA fragments were purified with Ampure XP (Beckman Coulter) prior to addition to Golden Gate Assembly Master Mix (New England Biolabs) and incubated per the manufacturer's instructions. The Golden Gate reaction was further purified and concentrated to enable maximum transformation efficiency in the subsequent steps of the bacterial screen.

The plasmid library containing the distinct repeat-spacer-repeat elements and CRISPR effectors was electroporated into E. Cloni electrocompetent E. coli (Lucigen) using a Gene Pulser Xcell® (Bio-rad) following the protocol recommended by Lucigen. The library was either co-transformed with purified pACYC184 plasmid or directly transformed into pACYC184-containing E. Cloni electrocompetent E. coli (Lucigen), plated onto agar containing chloramphenicol (Fisher), tetracycline (Alfa Aesar), and kanamycin (Alfa Aesar) in BioAssay® dishes (Thermo Fisher), and incubated for 10-12 hours at 37° C. After estimation of approximate colony count to ensure sufficient library representation on the bacterial plate, the bacteria were harvested, and plasmid DNA WAS extracted using a QIAprep Spin Miniprep® Kit (Qiagen) to create an “output library.” By performing a PCR using custom primers containing barcodes and sites compatible with Illumina sequencing chemistry, a barcoded next generation sequencing library was generated from both the pre-transformation “input library” and the post-harvest “output library,” which were then pooled and loaded onto a Nextseq 550 (Illumina) to evaluate the effectors. At least two independent biological replicates were performed for each screen to ensure consistency. See FIG. 1B.

### Bacterial Screen Sequencing Analysis

Next generation sequencing data for screen input and output libraries were demultiplexed using Illumina bcl2fastq. Reads in resulting fastq files for each sample contained the CRISPR array elements for the screening plasmid library. The direct repeat sequence of the CRISPR array was used to determine the array orientation, and the spacer sequence was mapped to the source (pACYC184 or E. Cloni) or negative control sequence (GFP) to determine the corresponding target. For each sample, the total number of reads for each unique array element (rₐ) in a given plasmid library was counted and normalized as follows: (rₐ+1)/total reads for all library array elements. The depletion score was calculated by dividing normalized output reads for a given array element by normalized input reads.

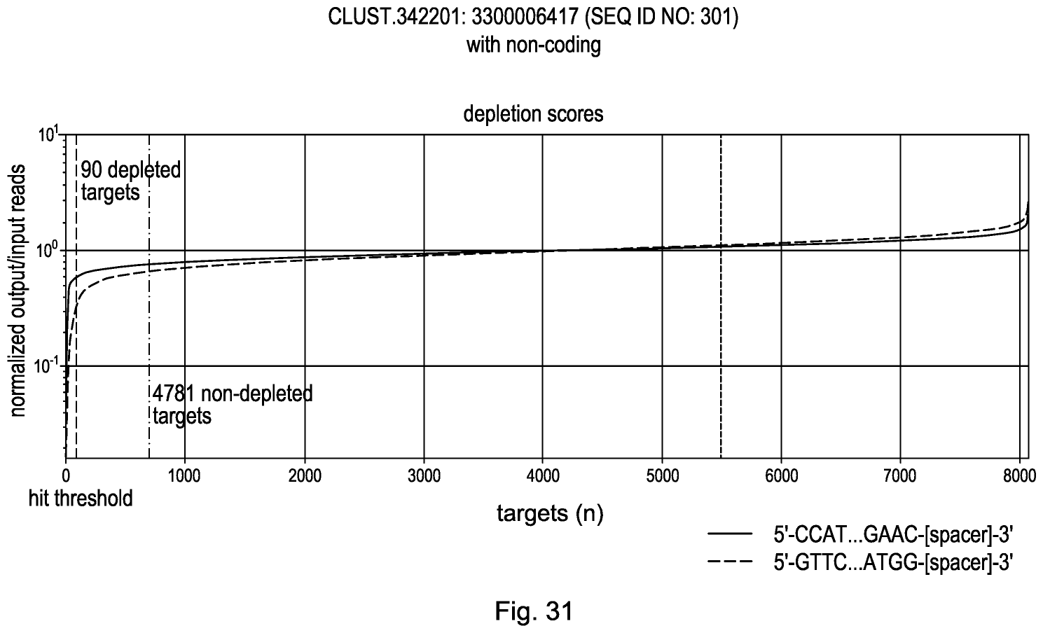

To identify specific parameters resulting in enzymatic activity and bacterial cell death, next generation sequencing (NGS) was used to quantify and compare the representation of individual CRISPR arrays (i.e., repeat-spacer-repeat) in the PCR product of the input and output plasmid libraries. The array depletion ratio was defined as the normalized output read count divided by the normalized input read count. An array was considered to be “strongly depleted” if the depletion ratio was less than 0.3 (more than 3-fold depletion), depicted by the blue dashed line in FIG. 31. When calculating the array depletion ratio across biological replicates, the maximum depletion ratio value for a given CRISPR array was taken across all experiments (i.e. a strongly depleted array must be strongly depleted in all biological replicates). A matrix including array depletion ratios and the following features were generated for each spacer target: target strand, transcript targeting, ORI targeting, target sequence motifs, flanking sequence motifs, and target secondary structure. The degree to which different features in this matrix explained target depletion for CLUST.342201 systems was investigated.

FIG. 31 shows the degree of interference activity of the engineered compositions, with a non-coding sequence, by plotting for a given target the normalized ratio of sequencing reads in the screen output versus the screen input. The results are plotted for each DR transcriptional orientation. In the functional screen for each composition, an active effector complexed with an active RNA guide will interfere with the ability of the pACYC184 to confer E. coli resistance to chloramphenicol and tetracycline, resulting in cell death and depletion of the spacer element within the pool. Comparison of the results of deep sequencing the initial DNA library (screen input) versus the surviving transformed E. coli (screen output) suggests specific target sequences and DR transcriptional orientations that enable an active, programmable CRISPR system. The screen also indicates that the effector complex is only active with one orientation of the DR. As such, the screen indicated that the CLUST.342201 3300006417 effector was active in the “reverse” orientation (5′-GTTC . . . ATGG-[spacer]-3′) of the DR (FIG. 31). The CLUST.342201 3300006417 effector did not retain activity in the absence of the non-coding sequence, indicating that CLUST.342201 effectors require a tracrRNA. Likewise, the negative control (plasmid without the effector) did not demonstrate activity.

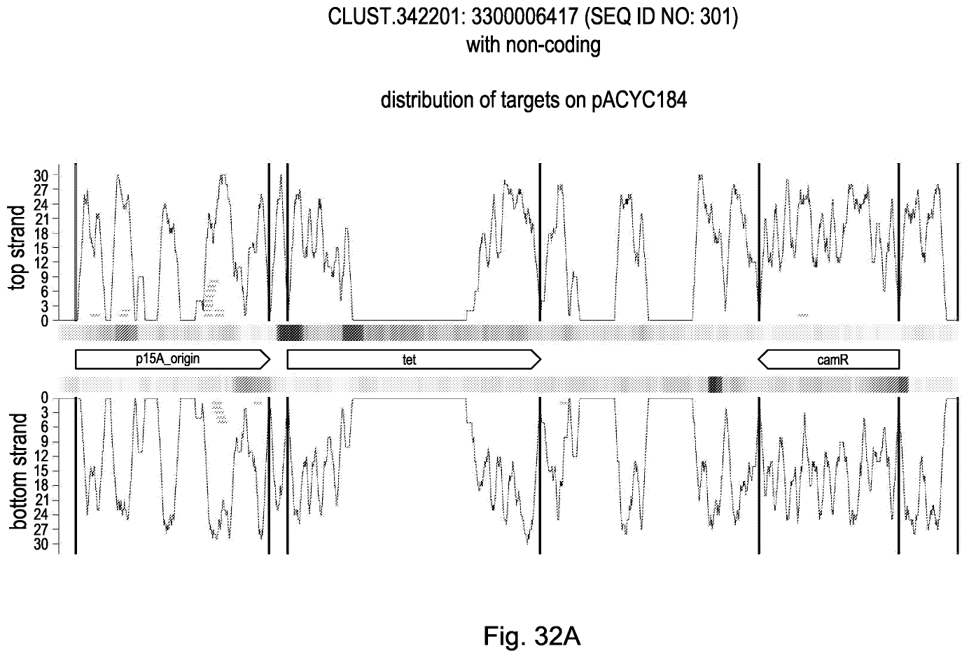

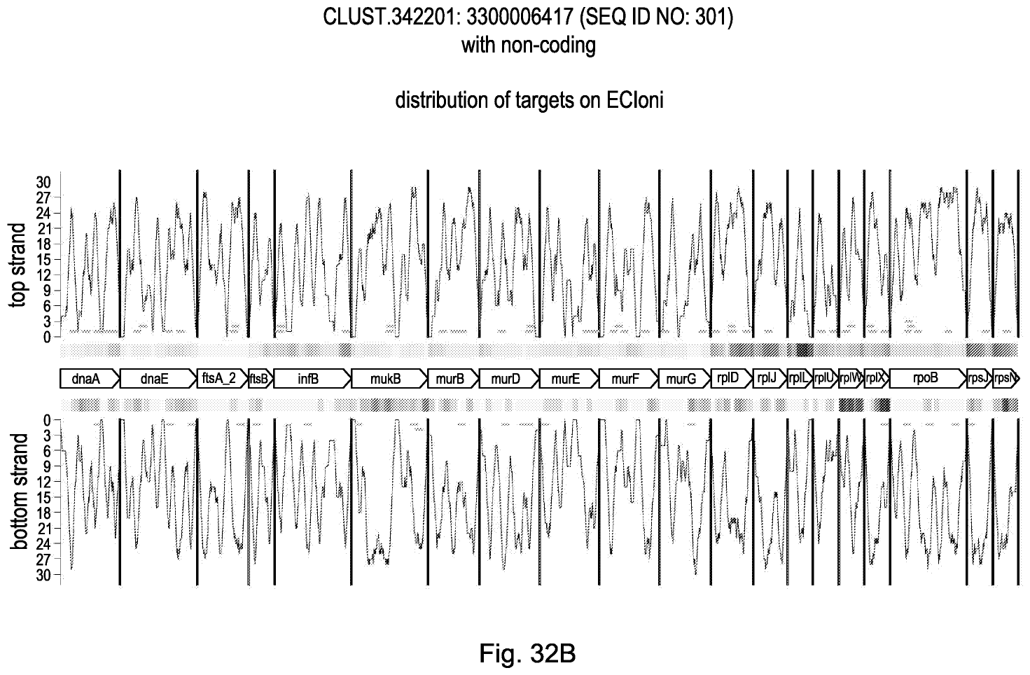

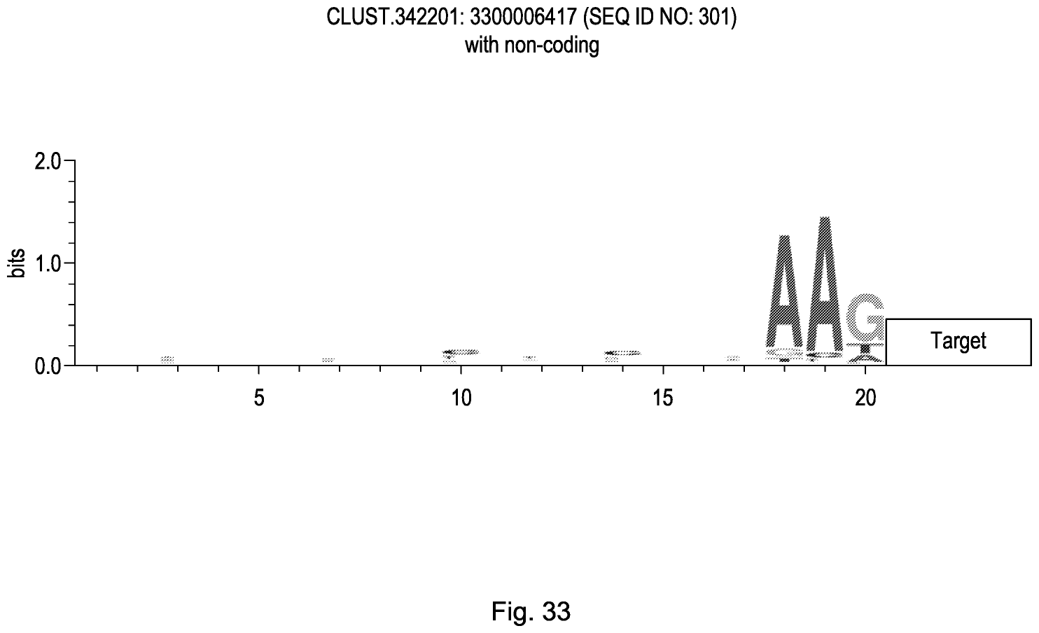

FIG. 32A and FIG. 32B depict the location of strongly depleted targets for the CLUST.342201 3300006417 effector (plus non-coding sequence) targeting pACYC184 and E. coli E. Cloni essential genes, respectively. Flanking sequences of depleted targets were analyzed to determine the PAM sequence for CLUST.342201 3300006417. A WebLogo representation (Crooks et al., Genome Research 14: 1188-90, 2004) of the PAM sequence for CLUST.342201 3300006417 is shown in FIG. 33.

### Example 11—Identification of Components of CLUST.195009 CRISPR-Cas System

This protein family was identified using the computational methods described above. The CLUST.195009 system comprises single effectors associated with CRISPR systems found in uncultured metagenomic sequences collected from environments not limited to hypersaline lake, aquatic, landfill, soil, and wastewater environments as well as from Acidobacteria (TABLE 22). Exemplary CLUST.195009 effectors include those shown in TABLES 22 and 23, below. Examples of direct repeat sequences and spacer lengths for these systems are shown in TABLE 24. Optionally, the system includes a tracrRNA that is contained in a non-coding sequence listed in TABLE 25.

TABLE 224
Representative CLUST.195009 Effector Proteins

| | | # | effector | SEQ |
|---|---|---|---|---|
| source | effector accession | spacers | size | ID NO |
| hypersaline lake metagenome | SRR6201554_1444827_42\|M | 14 | 560 | 501 |
| Acidobacteria bacterium RBG_13_68_16 | OFV81970.1 | 8 | 523 | 502 |
| (MEKI01000059) | | | | |
| aquatic-freshwater-groundwater | 3300009448\|Ga0114940_10001413_14\|P | 2 | 552 | 503 |
| aquatic-marine-estuarine | 3300001245\|JGI12048J13642_11056368_2\|M | 14 | 560 | 504 |
| aquatic-marine-estuarine | 3300001245\|JGI12048J13642_11056369_15\|M | 14 | 560 | 505 |
| aquatic-marine-seawater | 3300027881\|Ga0255055_10005864_11\|P | 5 | 541 | 506 |
| aquatic-non marine saline and alkaline- | 3300001256\|JGI12210J13797_10707440_22\|P | 6 | 559 | 507 |
| hypersaline mat | | | | |
| aquatic-non marine saline and alkaline- | 3300001256\|JGI12210J13797_10707440_21\|M | 6 | 471 | 508 |
| hypersaline mat | | | | |
| aquatic-non marine saline and alkaline- | 3300001256\|JGI12210J13797_10707441_3\|P | 6 | 559 | 509 |
| hypersaline mat | | | | |
| aquatic-non marine saline and alkaline- | 3300001256\|JGI12210J13797_10707441_2\|M | 6 | 471 | 510 |
| hypersaline mat) | | | | |
| aquatic-non marine saline and alkaline- | 3300001256\|JGI12210J13797_11264632_33\|M | 14 | 560 | 511 |
| hypersaline mat | | | | |
| aquatic-non marine saline and alkaline- | 3300001256\|JGI12210J13797_11264634_48\|M | 14 | 560 | 512 |
| hypersaline mat | | | | |
| aquatic-non marine saline and alkaline- | 3300001357\|JGI11876J14442_10000221_42\|P | 14 | 560 | 513 |
| hypersaline mat | | | | |
| hypersaline lake metagenome | SRR6201554_128554_7\|P | 4 | 559 | 514 |
| hypersaline lake metagenome | SRR6201554_128554_6\|M | 4 | 471 | 515 |
| solid waste-landfill-landfill leachate | 3300014204\|Ga0172381_10020148_1\|P | 2 | 545 | 516 |
| solid waste-landfill-landfill leachate | 3300028603\|Ga0265293_10119881_1\|M | 5 | 538 | 517 |
| terrestrial-soil-agricultural soil | 3300009095\|Ga0079224_100009227_18\|M | 4 | 524 | 518 |
| terrestrial-soil-agricultural soil | 3300009095\|Ga0079224_100214048_3\|P | 3 | 558 | 519 |
| wastewater-industrial wastewater- | 3300002220\|MLSBCLC_10211263_4\|M | 4 | 476 | 520 |
| hydrocarbon resource environments | | | | |
| wastewater-industrial wastewater- | 3300002220\|MLSBCLC_10211263_3\|P | 4 | 530 | 521 |
| hydrocarbon resource environments | | | | |

TABLE 235
Amino acid sequences of Representative CLUST.195009 Effector Proteins

>SRR6201554_1444827_42|M
[hypersaline lake metagenome]
MRQTLTLCRPARLISSEVRLDQEYRAARWAHHRLLDFEDEHQRHLNQVAEAIVPGIVRVGRVIARLSRRAKRRSRATRGSWTPD
PRPQLMTSLRARLVEMKKQRDADPRWKAALAWGDDAVGAPKKVRRRRAKDPSKVKRRKNESDEAWANRFAMLTSDETAEHFEAK
VAAAPRRSRREEYRAALYAQRRCYHGTWNALVRSVDQARSDVLRQRRSGLPADWRRPRFGGSQSICADRSGFRVVERGKPWWVV
EMRIGTASGKGAEWVRLRAKCGNWHAIPDDAAITTAKLTRRKDGQRWSYSLSLTVDGAQKAARAASSSGLVSFDWGHREHGHPR
ARDGIRAFVWLGDDGSTGEVIIPAECRQCLDEIDALKSRVDQAFDARRESMGLRDKNRHTYRRRVLRSGVRTEEEAQWLTWETR
YERRVARLRKRWQSLRREAYTRAVRELRTQYARFAFETESSASLKRQQRDEHMRRRARANRDLTARYEFVSLCERFGAEIIPVS
ARNTTKECPSCGHLGDNTSELVTVCPACGTARDKDVGAAEVILRRAEEALAKHSAE (SEQ ID NO: 501)
>OFV81970.1
[Acidobacteria bacterium RBG_13_68_16]
MTIRVIHRPAKVVDGLEALDREFRSARWAHHQLLRIEDDQAEQLDLVADMCAPGLRRVGRILAKLRRRACRAERATGWSPNPRV
ELRKTMAARMRSLKAQRDADPRWKIARGWLDASADDAPVKACRRKAGESDALYDARVARRKRRSRREEKRLELYSQIRCHYDTW
NELVKQVDQAIRSVRAVRKTGQSARWKHPRYDDPTTIASDHWDLVERGPVWWVIDLKLRAGVRVRLRTKAGTYDGVPADPDFRT
LKLTRRRVGRFGWEYSCSIAVNLAERVPSGTGVVALDWGHREHGHATADQGIRVFTWAGDDGQTGEVLLPIACRDLLSELDELK
GRLDTVYAARGEPELNRARYRRRLNMCGVLTREEQDWLRWETRQDRRLSAMRDRIEHIRRESYLKAIHELGRRYRVFAFEDLQG
QQIKDLQTENQMGRRKRQNRDLSARYLFESLCKQSGAERITVPARNSTRQCPACGKLREKTADLLVACDACGYVGDRDRDACRT
LLARAKEALAKRGASVQND (SEQ ID NO: 502)
>3300009448|Ga0114940_10001413_14|P
[aquatic-freshwater-groundwater]
MLTLNRPALLESDEQRLDAEYRSTNWAHHRLLDFEQEHQRVLDAAADEIAPGIIRAGRIVARLARRAKRADRTTGGQWFPPPRS
ELCTRLRVLLGELREQRNADPRWKLALGWADEQVGEPKAPRRRRAKPSSAVKRRKAETDDAFAKRFALLTSDESAEHYAAKLAT
PPRDSRRDMARKALYQQRRIYWGTWNALIRSVDQARKDVLDHRKRGMPADLRRPKFRDPSSIAADAGGFRIVERGALWWTIEMR
IGLGDEWARVRAKCGNWHAIPSDAKIGTAKLTRRRDGHRWSYSLSLTVDVAKNVDAHAPRGVVSFDWGHREHGHPSWRDGIRAF
TWLGDDGATGEVLIPIECRQALDEIDAMKARVDGAFNARKKAHDLPDVNRYTYRQRLMASGVRTEEQTAWLRWEMRYERRMDAR
RDRVQNLRKDAYTRAVRELRSRYAVFAFEDEDGASIRREQKEEQVLRRKRSNRDLATRYEFVALCERYGAELIAVPARNTTREC
PDCGELLKENGPELLVVCPGCGTARDKDHGAARVILGRAKEALAKRAA (SEQ ID NO: 503)
>3300001245|JGI12048J13642_11056368_2|M
[aquatic-marine-estuarine]
MRQTLTLCRPARLISSEVRLDQEYRAARWAHHRLLDFEDEHQRHLNQVAEAIVPGIVRVGRMIARLSRRAKRRSRATRGSWTPD
PRPQLMTSLRARLVEMKKQRDADPRWKAALAWGDDAVGAPKKVRRRRAKDPSKVKRRKNESDEAWANRFAMLTSDETAEHFEAK
VAAAPRRSRREEYRAALYAQRRCYHGTWNALVRSVDQARSDVLRQRRSGLPADWRRPRFGGSQSICADRSGFRVVERGKPWWVV
EMRIGTASGKGAEWVRLRAKCGNWHAIPDDAAITTAKLTRRKDGQRWSYSLSLTVDGAQKAARAASSSGLVSFDWGHREHGHPR
ARDGIRAFVWLGDDGSTGEVIIPAECRQCLDEIDALKSRVDQAFDARRESMGLRDKNRHTYRRRVLRSGVRTEEEAQWLTWETR
YERRVARLRKRWQSLRREAYTRAVRELRTQYARFAFETESSASLKRQQRDEHMRRRARANRDLTARYEFVSLCERFGAEIIPVS
ARNTTKECPSCGHLGDNTSELVTVCPACGTARDKDVGAAEVILRRAEEALAKHSAE (SEQ ID NO: 504)
>3300001245|JGI12048J13642_11056369_15|M
[aquatic-marine-estuarine]
MRQTLTLCRPARLISSEVRLDQEYRAARWAHHRLLDFEDEHQRHLNQVAEAIVPGIVRVGRMIARLSRRAKRRSRATRGSWTPD
PRPQLMTSLRARLVEMKKQRDADPRWKAALAWGDDAVGAPKKVRRRRAKDPSKVKRRKNESDEAWANRFAMLTSDETAEHFEAK
VAAAPRRSRREEYRAALYAQRRCYHGTWNALVRSVDQARSDVLRQRRSGLPADWRRPRFGGSQSICADRSGFRVVERGKPWWVV
EMRIGTASGKGAEWVRLRAKCGNWHAIPDDAAITTAKLTRRKDGQRWSYSLSLTVDGAQKAARAASSSGLVSFDWGHREHGHPR
ARDGIRAFVWLGDDGSTGEVIIPAECRQCLDEIDALKSRVDQAFDARRESMGLRDKNRHTYRRRVLRSGVRTEEEAQWLTWETR
YERRVARLRKRWQSLRREAYTRAVRELRTQYARFAFETESSASLKRQQRDEHMRRRARANRDLTARYEFVSLCERFGAEIIPVS
ARNTTKECPSCGHLGDNTSELVTVCPACGTARDKDVGAAEVILRRAEEALAKHSAE (SEQ ID NO: 505)
>3300027881|Ga0255055_10005864_11|P
[aquatic-marine-seawater]
MMKALARPARLVTGQAELDAEMRSARWIYHRLLDFEDAHQQVLDAAAEAVAPGISRVARLVARLLRRTRRRERTASSSWSPNPH
SAWLAGLRAQLNALRKVRNAHQVWRDAVKWADTPAPGAPERGGPRRRAGETAEEFAARCATRRTMLTRREAWRAELYRGHVADG
ARGRSRIYWGTWNSLLKSVDQARKSVLQARKAGLPANWRRPQWNDPSTIAADAGGFRVLERGRPWWALETRLCDGWVRFRAKCG
NWHSIPESAKLRTLKLTRRKNGCGWSYSVSIAVAGMPEPAHAGDGVVALDWGYREHGHPHERDGLRVFTWRGEDGAIGEILLPS
ECRAAKDREQQLQARIDAIWNARCASMKLPERNRHSYRSRLMRSGVRTAEEQRWLNWETRYERRRAAARKRWQNLRSETYLQAV
HALRRRYDMFAVENETIVGHRRTDKDEQTRHRKRQNRELSARYEFLQICERSGATILPVPSRNSTRECPHCGGLHENGPELYRA
CPATGVVDDKDEIACVTILARAKAALANRAVSAEKHA (SEQ ID NO: 506)
>3300001256|JGI12210J13797_10707440_22|P
[aquatic-non marine saline and alkaline-hypersaline mat]
MTILTLNRPALIISDDVRLDAEFRAARWAHHRLLDFEDQHQRHLDEEAERCAPGIVRVGRIIARLSRRAKRMSRATRGSWTPDP
RPQLMASLRVRLAEMKTQRDADPRWKAALAWADEPIGAPKKVRRRRAKDPSKVKRRKSETDEAFEKRFALFTSDETEEHYQKKV
AAAPRRTRREEYRAELYTKRRCYWGTWNALLRSVDQARSDVLTQRKKGLPADWQRPRFGDAQSLSAESGGFRIVERGKLWWVID
LRIGTATGKNAEWVRVRAKCGNWHHIPEGASITTAQLTRRKDGQRWSYSLSLTVDGAQKSAREVSNTGLVSFDWGHREHGHGRA
KEGIRAFVWIGDDGQQGEVILPAECRRCLDEIDALKSRMDQAFDARRESMGLREKNRHTYRRRLLRSGVQTDEEGQWLTWEMRY
ERRVGRLRKRWQNLRKETYVQAVRALRSRYAQFAFESESNASLKRQQKDEQMLRRARANRDLTARYEFVSLCERFGAEIVPVSA
RNTTKECPSCGHLGDNTAELVTVCPACGTARDKDFGAAKVIFRRAEGALAKHAAE (SEQ ID NO: 507)
>3300001256|JGI12210J13797_10707440_21|M
[aquatic-non marine saline and alkaline-hypersaline mat]
MASLRVRLAEMKTQRDADPRWKAALAWADEPIGAPKKVRRRRAKDPSKVKRRKSETDEAFEKRFALFTSDETEEHYQKKVAAAP
RRTRREEYRAELYTKRRCYWGTWNALLRSVDQARSDVLTQRKKGLPADWQRPRFGDAQSLSAESGGFRIVERGKLWWVIDLRIG
TATGKNAEWVRVRAKCGNWHHIPEGASITTAQLTRRKDGQRWSYSLSLTVDGAQKSAREVSNTGLVSFDWGHREHGHGRAKEGI
RAFVWIGDDGQQGEVILPAECRRCLDEIDALKSRMDQAFDARRESMGLREKNRHTYRRRLLRSGVQTDEEGQWLTWEMRYERRV
GRLRKRWQNLRKETYVQAVRALRSRYAQFAFESESNASLKRQQKDEQMLRRARANRDLTARYEFVSLCERFGAEIVPVSARNTT
KECPSCGHLGDNTAELVTVCPACGTARDKDFGAAKVIFRRAEGALAKHAAE (SEQ ID NO: 508)
>3300001256|JGI12210J13797_10707441_3|P
[aquatic-non marine saline and alkaline-hypersaline mat]
MTILTLNRPALIISDDVRLDAEFRAARWAHHRLLDFEDQHQRHLDEEAERCAPGIVRVGRIIARLSRRAKRMSRATRGSWTPDP
RPQLMASLRVRLAEMKTQRDADPRWKAALAWADEPIGAPKKVRRRRAKDPSKVKRRKSETDEAFEKRFALFTSDETEEHYQKKV
AAAPRRTRREEYRAELYTKRRCYWGTWNALLRSVDQARSDVLTQRKKGLPADWQRPRFGDAQSLSAESGGFRIVERGKLWWVID
LRIGTATGKNAEWVRVRAKCGNWHHIPEGASITTAQLTRRKDGQRWSYSLSLTVDGAQKSAREVSNTGLVSFDWGHREHGHGRA
KEGIRAFVWIGDDGQQGEVILPAECRRCLDEIDALKSRMDQAFDARRESMGLREKNRHTYRRRLLRSGVQTDEEGQWLTWEMRY
ERRVGRLRKRWQNLRKETYVQAVRALRSRYAQFAFESESNASLKRQQKDEQMLRRARANRDLTARYEFVSLCERFGAEIVPVSA
RNTTKECPSCGHLGDNTAELVTVCPACGTARDKDFGAAKVIFRRAEGALAKHAAE (SEQ ID NO: 509)
>3300001256|JGI12210J13797_10707441_2|M
[aquatic-non marine saline and alkaline-hypersaline mat]
MASLRVRLAEMKTQRDADPRWKAALAWADEPIGAPKKVRRRRAKDPSKVKRRKSETDEAFEKRFALFTSDETEEHYQKKVAAAP
RRTRREEYRAELYTKRRCYWGTWNALLRSVDQARSDVLTQRKKGLPADWQRPRFGDAQSLSAESGGFRIVERGKLWWVIDLRIG
TATGKNAEWVRVRAKCGNWHHIPEGASITTAQLTRRKDGQRWSYSLSLTVDGAQKSAREVSNTGLVSFDWGHREHGHGRAKEGI
RAFVWIGDDGQQGEVILPAECRRCLDEIDALKSRMDQAFDARRESMGLREKNRHTYRRRLLRSGVQTDEEGQWLTWEMRYERRV
GRLRKRWQNLRKETYVQAVRALRSRYAQFAFESESNASLKRQQKDEQMLRRARANRDLTARYEFVSLCERFGAEIVPVSARNTT
KECPSCGHLGDNTAELVTVCPACGTARDKDFGAAKVIFRRAEGALAKHAAE (SEQ ID NO: 510)
>3300001256|JGI12210J13797_11264632_33|M
[aquatic-non marine saline and alkaline-hypersaline mat]
MRQTLTLCRPARLISSEVRLDQEYRAARWAHHRLLDFEDEHQRHLNQVAEAIVPGIVRVGRMIARLSRRAKRRSRATRGSWTPD
PRPQLMTSLRARLVEMKKQRDADPRWKAALAWGDDAVGAPKKVRRRRAKDPSKVKRRKNESDEAWANRFAMLTSDETAEHFEAK
VAAAPRRSRREEYRAALYAQRRCYHGTWNALVRSVDQARSDVLRQRRSGLPADWRRPRFGGSQSICADRSGFRVVERGKPWWVV
EMRIGTASGKGAEWVRLRAKCGNWHAIPDDAAITTAKLTRRKDGQRWSYSLSLTVDGAQKAARAASSSGLVSFDWGHREHGHPR
ARDGIRAFVWLGDDGSTGEVIIPAECRQCLDEIDALKSRVDQAFDARRESMGLRDKNRHTYRRRVLRSGVRTEEEAQWLTWETR
YERRVARLRKRWQSLRREAYTRAVRELRTQYARFAFETESSASLKRQQRDEHMRRRARANRDLTARYEFVSLCERFGAEIIPVS
ARNTTKECPSCGHLGDNTSELVTVCPACGTARDKDVGAAEVILRRAEEALAKHSAE (SEQ ID NO: 511)
>3300001256|JGI12210J13797_11264634_48|M
[aquatic-non marine saline and alkaline-hypersaline mat]
MRQTLTLCRPARLISSEVRLDQEYRAARWAHHRLLDFEDEHQRHLNQVAEAIVPGIVRVGRMIARLSRRAKRRSRATRGSWTPD
PRPQLMTSLRARLVEMKKQRDADPRWKAALAWGDDAVGAPKKVRRRRAKDPSKVKRRKNESDEAWANRFAMLTSDETAEHFEAK
VAAAPRRSRREEYRAALYAQRRCYHGTWNALVRSVDQARSDVLRQRRSGLPADWRRPRFGGSQSICADRSGFRVVERGKPWWVV
EMRIGTASGKGAEWVRLRAKCGNWHAIPDDAAITTAKLTRRKDGQRWSYSLSLTVDGAQKAARAASSSGLVSFDWGHREHGHPR
ARDGIRAFVWLGDDGSTGEVIIPAECRQCLDEIDALKSRVDQAFDARRESMGLRDKNRHTYRRRVLRSGVRTEEEAQWLTWETR
YERRVARLRKRWQSLRREAYTRAVRELRTQYARFAFETESSASLKRQQRDEHMRRRARANRDLTARYEFVSLCERFGAEIIPVS
ARNTTKECPSCGHLGDNTSELVTVCPACGTARDKDVGAAEVILRRAEEALAKHSAE (SEQ ID NO: 512)
>3300001357|JGI11876J14442_10000221_42|P
[aquatic-non marine saline and alkaline-hypersaline mat]
MRQTLTLCRPARLISSEVRLDQEYRAARWAHHRLLDFEDEHQRHLNQVAEAIVPGIVRVGRMIARLSRRAKRRSRATRGSWTPD
PRPQLMTSLRARLVEMKKQRDADPRWKAALAWGDDAVGAPKKVRRRRAKDPSKVKRRKNESDEAWANRFAMLTSDETAEHFEAK
VAAAPRRSRREEYRAALYAQRRCYHGTWNALVRSVDQARSDVLRQRRSGLPADWRRPRFGGSQSICADRSGFRVVERGKPWWVV
EMRIGTASGKGAEWVRLRAKCGNWHAIPDDAAITTAKLTRRKDGQRWSYSLSLTVDGAQKAARAASSSGLVSFDWGHREHGHPR
ARDGIRAFVWLGDDGSTGEVIIPAECRQCLDEIDALKSRVDQAFDARRESMGLRDKNRHTYRRRVLRSGVRTEEEAQWLTWETR
YERRVARLRKRWQSLRREAYTRAVRELRTQYARFAFETESSASLKRQQRDEHMRRRARANRDLTARYEFVSLCERFGAEIIPVS
ARNTTKECPSCGHLGDNTSELVTVCPACGTARDKDVGAAEVILRRAEEALAKHSAE (SEQ ID NO: 513)
>SRR6201554_128554_7|P
[hypersaline lake metagenome]
MTILTLNRPALIISDDVRLDAEFRAARWAHHRLLDFEDQHQRHLDEEAERCAPGIVRVGRIIARLSRRAKRMSRATRGSWTPDP
RPQLMASLRVRLAEMKTQRDADPRWKAALAWADEPIGAPKKVRRRRAKDPSKVKRRKSETDEAFEKRFALFTSDETEEHYQKKV
AAAPRRTRREEYRAELYTKRRCYWGTWNALLRSVDQARSDVLTQRKKGLPADWQRPRFGDAQSLSAESGGFRIVERGKLWWVID
LRIGTATGKNAEWVRVRAKCGNWHHIPEGASITTAQLTRRKDGQRWSYSLSLTVDGAQKSAREVSNTGLVSFDWGHREHGHGRA
KEGIRAFVWIGDDGQQGEVILPAECRRCLDEIDALKSRMDQAFDARRESMGLREKNRHTYRRRLLRSGVQTDEEGQWLTWEMRY
ERRVGRLRKRWQNLRKETYVQAVRALRSRYAQFAFESESNASLKRQQKDEQMLRRARANRDLTARYEFVSLCERFGAEIVPVSA
RNTTKECPSCGHLGDNTAELVTVCPACGTARDKDFGAAKVIFRRAEGALAKHAAE (SEQ ID NO: 514)
>SRR6201554_128554_6|M
[hypersaline lake metagenome]
MASLRVRLAEMKTQRDADPRWKAALAWADEPIGAPKKVRRRRAKDPSKVKRRKSETDEAFEKRFALFTSDETEEHYQKKVAAAP
RRTRREEYRAELYTKRRCYWGTWNALLRSVDQARSDVLTQRKKGLPADWQRPRFGDAQSLSAESGGFRIVERGKLWWVIDLRIG
TATGKNAEWVRVRAKCGNWHHIPEGASITTAQLTRRKDGQRWSYSLSLTVDGAQKSAREVSNTGLVSFDWGHREHGHGRAKEGI
RAFVWIGDDGQQGEVILPAECRRCLDEIDALKSRMDQAFDARRESMGLREKNRHTYRRRLLRSGVQTDEEGQWLTWEMRYERRV
GRLRKRWQNLRKETYVQAVRALRSRYAQFAFESESNASLKRQQKDEQMLRRARANRDLTARYEFVSLCERFGAEIVPVSARNTT
KECPSCGHLGDNTAELVTVCPACGTARDKDFGAAKVIFRRAEGALAKHAAE (SEQ ID NO: 515)
>3300014204|Ga0172381_10020148_1|P
[solid waste-landfill-landfill leachate]
MKTLSRPAVIVDGIDDLGREYSSARWIYHRLLDFEDTHQSVLAAAAERAAPGITRVGRIVSRLSRRIRWSERSTGWSPPLHTPW
LTSLRARLIELRAERNASDGWREAMCWADDSEPGAPARGGARRKAGETDEDFAARSAKRRTTLTRREAHRAALYAEHVAVGSSG
RSRIHWGTWNGLVKSVDQARAAVLKARKQGLPAEWRRPRWSDDSTLFADTHGFRIVSKDDSGPWWTLELRTHFGWVRFRAKCGN
WHEIPESASMRTLKLTRRQKTLGSSGRSTRKGWAYSVSIAIEGVEPAAHTGAGLVALDWGHREHGHPHESEGIRAFTWLGDDGA
LGEILLPRECREATDAVDEMKARVDGAFNARKSALGLPDRNRHRYRSRLMRAGVRTDEEQLWLTWEDRYEMRMQRARKRVKNLR
QETYLKAVRMLRSRYSQFAIEDESIMRHRAADIDKETSHRKRSNRELTARYLFVSICERLGAEMIPVPARNSTRECPKCGHLDE
NTAELIIVCGGCGTARDKDHGAVQVILRRAKEALEKRLASP (SEQ ID NO: 516)
>3300028603|Ga0265293_10119881_1|M
[solid waste-landfill-landfill leachate]
MKTLSRPAVVDRDTATRLDREYTAARWLHNRILDFEAEHQRVIDAAADTAAPGISRCARIVAKLRGRVRRRERSTGWSPPLHEE
WRKILEARIAELRKARNADPAYQAALRWDRESAPGAVVKLPRRKAGETDEAFAARKAAGKTRSRREAWRTEVCYPQRRCFISSY
NALCRIVDQARSAVLAERKAGRPAEWRRPRHSDPTATLHWEPGSWEIVDMGPVWWTIRLAVGYSDRQTKADHVTVRAKCGTWHD
TTGREFATAKLTRIRDGRGYRYSLSLCVDVAAAPLPGDGVVGLDWGHREHGHPEQDEGIRAWSWHGDDGRSGVVILPAEIREQL
DRVHELQSRIDTVWDARRREQKLPERSRQGYRTRLLREGAPTAEEAAWLDWETRYERRAQRSRQRVQALRRETYLRAVRELRQR
YRVFAVEDQPGRAHRDLDTEEQTRRRKRENRDVVARYEFLSICERLGADVIPVPARDTTRECPDCGHLAERTSDLTIACVSCGR
VRDRDAGAARVILRRGIEALANRAAKARNSEAAE (SEQ ID NO: 517)
>3300009095|Ga0079224_100009227_18|M
[terrestrial-soil-agricultural soil]
MIFAIKRPGILESDPSALDTELRAARRTHHLLLDFEDKHQALLDEVAETVAPGIGRIARIVNKLISRKHRMERSTGWSPPLHDN
WRLVLKDRLQSLRNQRNSDPRWKTALKWADMPGENAPPRGGARRKVGETDEAFAARVSSRRTVLTRREAYRAELYTGRTIYWGT
WNGLLKQVDQARSMVVSQRAAGLPAEWHRPRWDKPTTLYADRGGFTVHRENKHWVVDIRLLKGSARFRLSTRGKISDNFTEFRT
AKVTRFKNGAGWSYSVSLTVSSESSLPAVGAGVVAFDGGYRERGHDAEQQGIRALVWTGNDGKSGEVLLPLACRTLADQNKQLL
SKLDTEFTQLNVPFRSRHHYRSALMRAGVRTELEHAWLGDERRIERRVAANRKRIENLRKETYLRVVKELLASYGTFVFEDTSG
QALRDIGTDKQALRRSRQNRDMVAEYSFRKLIERRGGNVITVTARNSTRECPTCGYLCESTAQISIKCPACSTIRDQDFGASQV
LLRRGLEALEKHNSNTRIAS (SEQ ID NO: 518)
>3300009095|Ga0079224_100214048_3|P
[terrestrial-soil-agricultural soil]
MLVLHRPAKLESDERRLDQEYRSARWLHHRILDFEDEHQRVLDTVAEQIAPGVVRIGQILARLNGRARRAERSSKGVWCPNPHP
DLAERLKARLQELRKVRDADPRWKAATKWPDEEIGEPKQVRRRRAKPASKIQRRKSESEEAFRKRFELLTQDESDEHYAQKLAN
ARRDSRRDVHRKKLYAERRIYWGTWNSLCERVDAARQAVLKRRKQGLPAEWRRPRWDQSNTIAAPAAGFRIVEQGPLWWTAEMR
LGTGDGNEAEWVRFRFKGGNWHKLGPEAKLVACELTRRRDGMRWNYSVSITVDGVTKASAPAATTRCVAFDWGHREHGHDRARE
GIRAFVWVGDDGRSGEVLIPRECREAQDEIDALKSRVDTAFLARKQSLELLDHTRYSYRRRLTRSGCCTREESAWLRWEQRYER
RIQKRRKRIDNLREETYLRTVQELRSHYAFFGFEDESGQGLRRKQADDQMARRKRANRDLATRFEFVSLCERFGAEVVTVPARN
TTRECPSCGHVGENGPELVTVCQACGTARDKDFGAAMVILKRMQQALANRDAAA (SEQ ID NO: 519)
>3300002220|MLSBCLC_10211263_4|M
[wastewater-industrial wastewater-hydrocarbon resource environments]
MRVGRIVARLLRRSKRRARASIGTWTPDPRPALLVSLKARLDELREQRNADPRWREACRWADTPADDAPVRGEARRKASETDED
FAKRCEKRRDRLTRREAYRHALYEQRRCYWGTYNALCKCVDQARVSVIKARKAGLPAEWRRPRFPQSPQSIWADKGFRIVERGK
LWWTLEMPLGVKPIQWVRFRAKCGNWHAIPEDAHLRMVQLRRERDGHRWRYSVSIVIAGIPERRHNGSGVVGLDWGHREHGHGL
EREGIRAFTWIGDDGASGEILIPRECRDQLDRIDELKARADDAFNARGLPDRNRYSYRRRLMGLGVRTHEESLWLQWEMRYERR
MASARKRWQALRQGVYLSAVRQLRQRYAVFAFEDETGRGHRKLDTEEQTRHRKRSNRDLTARYEFLQICERSGARVITVPARNS
TRECPEPECGGLLPENGPELLVACPKCGRVRDKDYGAARVILRRALEALATEAQSA (SEQ ID NO: 520)
>3300002220|MLSBCLC_10211263_3|P
[wastewater-industrial wastewater-hydrocarbon resource environments]
MVTLALKRPAIVDATEALDAEYRSARWLHHRLLDFEDEHQRLLDCVANECAPGVVRVGRIVARLLRRSKRRARASIGTWTPDPR
PALLVSLKARLDELREQRNADPRWREACRWADTPADDAPVRGEARRKASETDEDFAKRCEKRRDRLTRREAYRHALYEQRRCYW
GTYNALCKCVDQARVSVIKARKAGLPAEWRRPRFPQSPQSIWADKGFRIVERGKLWWTLEMPLGVKPIQWVRFRAKCGNWHAIP
EDAHLRMVQLRRERDGHRWRYSVSIVIAGIPERRHNGSGVVGLDWGHREHGHGLEREGIRAFTWIGDDGASGEILIPRECRDQL
DRIDELKARADDAFNARGLPDRNRYSYRRRLMGLGVRTHEESLWLQWEMRYERRMASARKRWQALRQGVYLSAVRQLRQRYAVF
AFEDETGRGHRKLDTEEQTRHRKRSNRDLTARYEFLQICERSGARVITVPARNSTRECPEPECGGLLPENGPELLVACPKCGRV
RDKDYGAARVILRRALEALATEAQSA (SEQ ID NO: 521)

TABLE 246
Nucleotide Sequences of Representative CLUST.195009 Direct Repeats and Spacer
Lengths

| | | Spacer |
|---|---|---|
| CLUST.195009 Effector Protein Accession | Direct Repeat Nucleotide Sequence | Length(s) |
| SRR6201554_1444827_42\|M (SEQ ID NO: 501) | CCAGCAACAGCCGCGTGGGGCTACTAGTACTGCGAC (SEQ | 24-39 |
| | ID NO: 522) | |
| | GTCGCAGTACTAGTAGCCCCACGCGGCTGTTGCTGG (SEQ | |
| | ID NO: 539) | |
| OFV81970.1 (SEQ ID NO: 502) | CCACCAACAGCCGCGCAGGGCTTCGAATACTGCGAC (SEQ | 33-54 |
| | ID NO: 523) | |
| | GTCGCAGTATTCGAAGCCCTGCGCGGCTGTTGGTGG (SEQ | |
| | ID NO: 540) | |
| 3300009448\|Ga0114940_10001413_14\|P | CCAACAACAGCCGCGCAGGGCTTCGAGTACTGCGAC (SEQ | 37-38 |
| (SEQ ID NO: 503 ) | ID NO: 524) | |
| | GTCGCAGTACTCGAAGCCCTGCGCGGCTGTTGTTGG (SEQ | |
| | ID NO: 541) | |
| 3300001245\|JGI12048J13642_11056368_2\|M | CCAGCAACAGCCGCGTGGGGCTACTAGTACTGCGAC (SEQ | 23-39 |
| (SEQ ID NO: 504) | ID NO: 522) | |
| | GTCGCAGTACTAGTAGCCCCACGCGGCTGTTGCTGG (SEQ | |
| | ID NO: 539) | |
| 3300001245\|JGI12048J13642_11056369_15\|M | CCAGCAACAGCCGCGTGGGGCTACTAGTACTGCGAC (SEQ | 24-39 |
| (SEQ ID NO: 505) | ID NO: 522) | |
| | GTCGCAGTACTAGTAGCCCCACGCGGCTGTTGCTGG (SEQ | |
| | ID NO: 539) | |
| 3300027881\|Ga0255055_10005864_11\|P | CTCCCACCAGCCGCGCAGGGCTACGAGTACTGCGAC (SEQ | 35-37 |
| (SEQ ID NO: 506) | ID NO: 525) | |
| | GTCGCAGTACTCGAAGCCCTGCGCGGCTGTTGTTGG (SEQ | |
| | ID NO: 542) | |
| 3300001256\|JGI12210J13797_10707440_22\|P | CCAGCAACAGCCGCGTGGGGCTTCAAATACTGCGAC (SEQ | 37-39 |
| (SEQ ID NO: 507) | ID NO: 526) | |
| | GTCGCAGTATTTGAAGCCCCACGCGGCTGTTGCTGG (SEQ | |
| | ID NO: 543) | |
| 3300001256\|JGI12210J13797_10707440_21\|M | CCAGCAACAGCCGCGTGGGGCTTCAAATACTGCGAC (SEQ | 37-39 |
| (SEQ ID NO: 508) | ID NO: 526) | |
| | GTCGCAGTATTTGAAGCCCCACGCGGCTGTTGCTGG (SEQ | |
| | ID NO: 543) | |
| 3300001256\|JGI12210J13797_10707441_3\|P | CCAGCAACAGCCGCGTGGGGCTTCAAATACTGCGAC (SEQ | 37-39 |
| (SEQ ID NO: 509) | ID NO: 526) | |
| | GTCGCAGTATTTGAAGCCCCACGCGGCTGTTGCTGG (SEQ | |
| | ID NO: 543) | |
| 3300001256\|JGI12210J13797_10707441_2\|M | CCAGCAACAGCCGCGTGGGGCTTCAAATACTGCGAC (SEQ | 37-39 |
| (SEQ ID NO: 510) | ID NO: 526) | |
| | GTCGCAGTATTTGAAGCCCCACGCGGCTGTTGCTGG (SEQ | |
| | ID NO: 543) | |
| 3300001256\|JGI12210J13797_11264632_33\|M | CCAGCAACAGCCGCGTGGGGCTACTAGTACTGCGAC (SEQ | 23-39 |
| (SEQ ID NO: 511) | ID NO: 522) | |
| | GTCGCAGTACTAGTAGCCCCACGCGGCTGTTGCTGG (SEQ | |
| | ID NO: 539) | |
| 3300001256\|JGI12210J13797_11264634_48\|M | CCAGCAACAGCCGCGTGGGGCTACTAGTACTGCGAC (SEQ | 24-39 |
| (SEQ ID NO: 512) | ID NO: 522) | |
| | GTCGCAGTACTAGTAGCCCCACGCGGCTGTTGCTGG (SEQ | |
| | ID NO: 539) | |
| 3300001357\|JGI11876J14442_10000221_42\|P | CCAGCAACAGCCGCGTGGGGCTACTAGTACTGCGAC (SEQ | 24-39 |
| (SEQ ID NO: 513) | ID NO: 522) | |
| | GTCGCAGTACTAGTAGCCCCACGCGGCTGTTGCTGG (SEQ | |
| | ID NO: 539) | |
| SRR6201554_128554_71P (SEQ ID NO: 514) | CCAGCAACAGCCGCGTGGGGCTGCAAATACTGCGAC (SEQ | 37-39 |
| | ID NO: 527) | |
| | GTCGCAGTATTTGCAGCCCCACGCGGCTGTTGCTGG (SEQ | |
| | ID NO: 544) | |
| SRR6201554_128554_6\|M (SEQIDNO: 515) | CCAGCAACAGCCGCGTGGGGCTGCAAATACTGCGAC (SEQ | 37-39 |
| | ID NO: 527) | |
| | GTCGCAGTATTTGCAGCCCCACGCGGCTGTTGCTGG (SEQ | |
| | ID NO: 544) | |
| 3300014204\|Ga0172381_10020148_1\|P | CTCGCACACCAGCCGCTCAGGGCTTCGTACTGAGAC (SEQ | 35-38 |
| (SEQ ID NO: 516) | ID NO: 528) | |
| | GTCTCAGTACGAAGCCCTGAGCGGCTGGTGTGCGAG (SEQ | |
| | ID NO: 545) | |
| 3300028603\|Ga0265293_10119881_1\|M | CCAACAACAGCCGCGCGGGGCTGCGAATACTGCGAC (SEQ | 36-37 |
| (SEQ ID NO: 517) | ID NO: 529) | |
| | GTCGCAGTATTCGCAGCCCCGCGCGGCTGTTGTTGG (SEQ | |
| | ID NO: 546) | |
| 3300009095\|Ga0079224_100009227_18\|M | CTTCCAACAGCCGTGCAGGGCTTCGAATGCTGCA (SEQ | 37-44 |
| (SEQ ID NO: 518) | ID NO: 530) | |
| | TGCAGCATTCGAAGCCCTGCACGGCTGTTGGAAG (SEQ | |
| | ID NO: 547) | |
| 3300009095\|Ga0079224_100214048_3\|P | CCACCAACAGCCGCCGAGGGCTTCAAATTCTCGGACG | 35 |
| (SEQ ID NO: 519) | (SEQ ID NO: 531) | |
| | CGTCCGAGAATTTGAAGCCCTCGGCGGCTGTTGGTGG | |
| | (SEQ ID NO: 548) | |
| 33000022201\|MLSBCLC_10211263_4\|M | GATCACAACAGCCGCGAGAGGCTTCGAATATCTCGAC | 35-37 |
| (SEQ ID NO: 520) | (SEQ ID NO: 532) | |
| | GTCGAGATATTCGAAGCCTCTCGCGGCTGTTGTGATC | |
| | (SEQ ID NO: 549) | |
| 33000022201\|MLSBCLC_10211263_3\|P | GATCACAACAGCCGCGAGAGGCTTCGAATATCTCGAC | 35-37 |
| (SEQ ID NO: 521) | (SEQ ID NO: 532) | |
| | GTCGAGATATTCGAAGCCTCTCGCGGCTGTTGTGATC | |
| | (SEQ ID NO: 549) | |

TABLE 257
Non-coding Sequences of Representative CLUST.195009 Systems

>SRR6201554_1444827_42|M
GCCGACATCGCCGACACGGCCTACCTCATGCTGAGGCCTTTGACGGGCAAGCGCGCCCGCACGATCGACGAGGAGAACGCGCAG
CCGGGCCGCATGGACCCCCGCAACATCGGCAACGTCGTCGCCGAGGGGCAGAGCAAGAACCTATGAGCTGCTGCTGCTCCGATG
AGATCTGCCCGGTCTGGGAGGAAACCACCCCCCGGGCCCGAAGAGCCTACACGTGCTGCGAGTGCAGCGGCCCCATTCGCGTGG
GGGAACGTCACCACAAGATCTCTTCCCTTTTCGATGGGTCGTGGAGCACATCGCGTTCGCACGAGCGCTGTCATGCCATCAGCA
GCCTGCTGCAGGCGGACTGCGACGTGTACTGCGTAGGGATGCTTCGTGAGTGCCTCCATGCGCAGTCGGCCGGGCACCTTCCGG
AGGACCTTCGGCCTTACGCCAAGGGGCTCATGTTCAAGTTCTCGGAGCACTACTACGAGCACCAAGCGCGACTGCTGTCGAAAC
GTCGCTACGGGGCCCAGGCCGTATCCTCAAGGTCGGTTTCATGAGGCAGACTCTGACGCTGTGCCGGCCAGCCAGGTTGATCTC
CAGCGAAGTGCGGCTGGACCAGGAGTACCGGGCAGCCCGCTGGGCTCACCACCGCCTGCTGGACTTCGAGGACGAGCACCAGCG
ACACCTCAATCAGGTGGCAGAATGCCCATCGTGTGGTCACCTTGGTGATAATACGTCGGAATTGGTGACGGTGTGTCCGGCTTG
CGGCACCGCCAGGGACAAGGACGTTGGCGCGGCCGAGGTGATCCTTCGGCGCGCAGAGGAGGCGCTCGCAAAGCATTCTGCAGA
GTAGCGAAATCAAAGGGGTCGCGAGGCGTCCCCTCAAGGGACACCGCACCAACAGCCGCGTGGGGCTTCCAATACCTCGACAGT
CGCCCCACCGCTCGACGTGCGAACCCCACCGCTTCTGCGACTCGCCCCCTCACCCTAGCCCTTCACGCTGCCCCTCTCATGCTG
CCAACAAGCTCTTCGAAGCTCTGTCGCTCCAGCGCCTCCTCGTCAGGCCGGTCGCGGTCGTCGACCACGGCGTCGATGATACGT
TGTTTGGCGGCCAACACGCGCCCGATGTGCTCGTCGATGGTGTC (SEQ ID NO: 533)
>3300028603|Ga0265293_10119881_1|M
GAGTGAAGACACTATCCAGACCAGCGGTCGTAGACCGCGATACAGCGACCAGGCTCGACCGTGAGTACACGGCGGCACGATGGC
TGCACAACCGCATCCTCGACTTCGAGGCGGAGCACCAGCGGGTGATCGATGCCGCGGCAGACACCGCTGAGAGGACTAGCGATC
TAACCATAGCGTGCGTATCATGTGGACGTGTACGCGACCGTGATGCGGGGGCTGCCCGCGTCATCCTACGGCGCGGAATCGAGG
CGCTCGCAAACCGTGCCGCAAAGGCGCGGAACAGCGAGGCAGCAGAGTGAACAAA (SEQ ID NO: 534)

### Example 12—Functional Validation of an Engineered CLUST.195009 CRISPR-Cas System

Having identified components of CLUST.195009 CRISPR-Cas systems, a locus from the metagenomic source designated SRR6201554 (SEQ ID NO: 501) was selected for functional validation.

### DNA Synthesis and Effector Library Cloning

To test the activity of the exemplary CLUST.195009 CRISPR-Cas systems, systems were designed and synthesized using a pET28a(+) vector. Briefly, an E. coli codon-optimized nucleic acid sequence encoding the CLUST.195009 SRR6201554 effector (SEQ ID NO: 501 shown in TABLE 23) was synthesized (Genscript) and cloned into a custom expression system derived from pET-28a(+) (EMD-Millipore). The vector included the nucleic acid encoding the CLUST.195009 effector under the control of a lac promoter and an E. coli ribosome binding sequence. The vector also included an acceptor site for a CRISPR array library driven by a J23119 promoter following the open reading frame for the CLUST.195009 effector. The non-coding sequence used for the CLUST.195009 SRR6201554 effector (SEQ ID NO: 501) is set forth in SEQ ID NO: 533, as shown in TABLE 25. An additional condition was tested, wherein the CLUST.195009 SRR6201554 effector (SEQ ID NO: 501) was individually cloned into pET28a(+) without the non-coding sequence. See FIG. 1A.

An oligonucleotide library synthesis (OLS) pool containing “repeat-spacer-repeat” sequences was computationally designed, where “repeat” represents the consensus direct repeat sequence found in the CRISPR array associated with the effector, and “spacer” represents sequences tiling the pACYC184 plasmid or E. coli essential genes. In particular, the repeat sequence used for the CLUST.195009 SRR6201554 effector (SEQ ID NO: 501) is set forth in SEQ ID NO: 522, as shown in TABLE 24. The spacer length was determined by the mode of the spacer lengths found in the endogenous CRISPR array. The repeat-spacer-repeat sequence was appended with restriction sites enabling the bi-directional cloning of the fragment into the aforementioned CRISPR array library acceptor site, as well as unique PCR priming sites to enable specific amplification of a specific repeat-spacer-repeat library from a larger pool.

Next, the repeat-spacer-repeat library was cloned into the plasmid using the Golden Gate assembly method. Briefly, each repeat-spacer-repeat was first amplified from the OLS pool (Agilent Genomics) using unique PCR primers and pre-linearized the plasmid backbone using BsaI to reduce potential background. Both DNA fragments were purified with Ampure XP (Beckman Coulter) prior to addition to Golden Gate Assembly Master Mix (New England Biolabs) and incubated per the manufacturer's instructions. The Golden Gate reaction was further purified and concentrated to enable maximum transformation efficiency in the subsequent steps of the bacterial screen.

The plasmid library containing the distinct repeat-spacer-repeat elements and CRISPR effectors was electroporated into E. Cloni electrocompetent E. coli (Lucigen) using a Gene Pulser Xcell® (Bio-rad) following the protocol recommended by Lucigen. The library was either co-transformed with purified pACYC184 plasmid or directly transformed into pACYC184-containing E. Cloni electrocompetent E. coli (Lucigen), plated onto agar containing chloramphenicol (Fisher), tetracycline (Alfa Aesar), and kanamycin (Alfa Aesar) in BioAssay® dishes (Thermo Fisher), and incubated for 10-12 hours at 37° C. After estimation of approximate colony count to ensure sufficient library representation on the bacterial plate, the bacteria were harvested, and plasmid DNA WAS extracted using a QIAprep Spin Miniprep® Kit (Qiagen) to create an “output library.” By performing a PCR using custom primers containing barcodes and sites compatible with Illumina sequencing chemistry, a barcoded next generation sequencing library was generated from both the pre-transformation “input library” and the post-harvest “output library,” which were then pooled and loaded onto a Nextseq 550 (Illumina) to evaluate the effectors. At least two independent biological replicates were performed for each screen to ensure consistency. See FIG. 1B.

### Bacterial Screen Sequencing Analysis

Next generation sequencing data for screen input and output libraries were demultiplexed using Illumina bcl2fastq. Reads in resulting fastq files for each sample contained the CRISPR array elements for the screening plasmid library. The direct repeat sequence of the CRISPR array was used to determine the array orientation, and the spacer sequence was mapped to the source (pACYC184 or E. Cloni) or negative control sequence (GFP) to determine the corresponding target. For each sample, the total number of reads for each unique array element (rₐ) in a given plasmid library was counted and normalized as follows: (rₐ+1)/total reads for all library array elements. The depletion score was calculated by dividing normalized output reads for a given array element by normalized input reads.

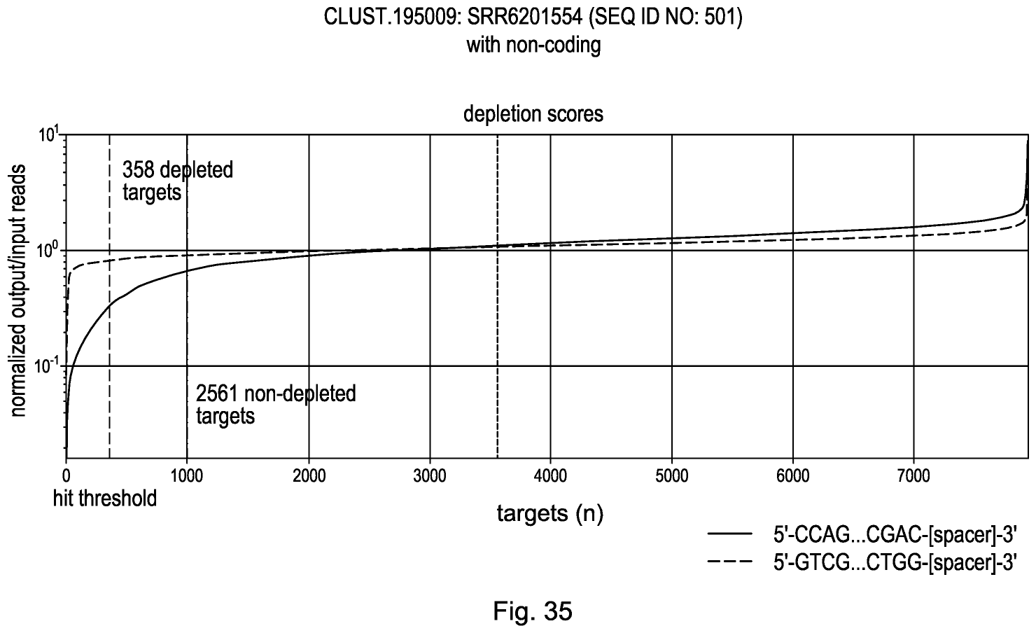

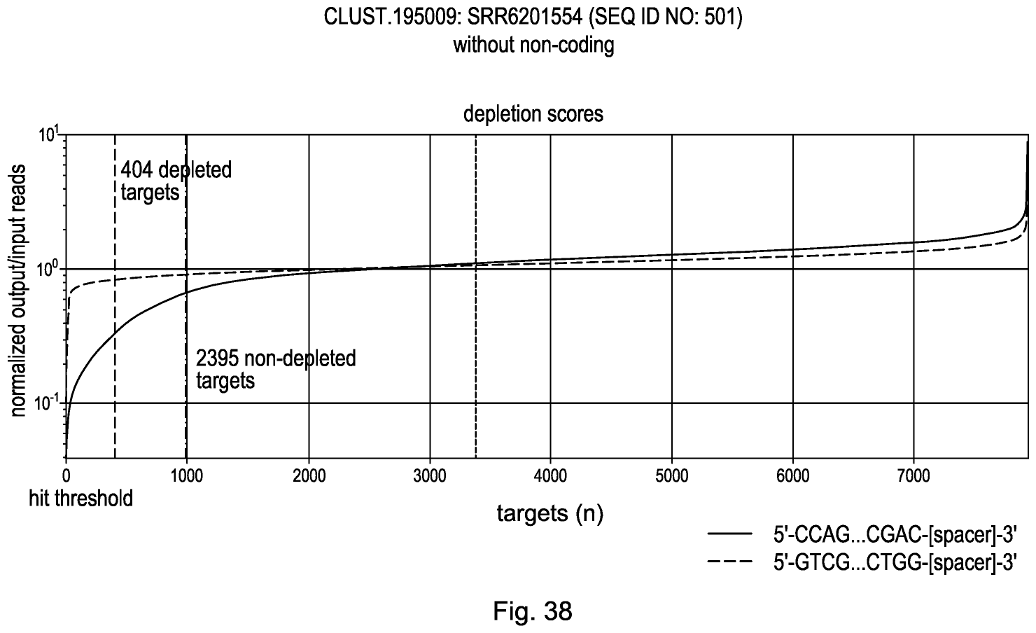

To identify specific parameters resulting in enzymatic activity and bacterial cell death, next generation sequencing (NGS) was used to quantify and compare the representation of individual CRISPR arrays (i.e., repeat-spacer-repeat) in the PCR product of the input and output plasmid libraries. The array depletion ratio was defined as the normalized output read count divided by the normalized input read count. An array was considered to be “strongly depleted” if the depletion ratio was less than 0.3 (more than 3-fold depletion), depicted by the blue dashed line in FIG. 35 and FIG. 38. When calculating the array depletion ratio across biological replicates, the maximum depletion ratio value for a given CRISPR array was taken across all experiments (i.e. a strongly depleted array must be strongly depleted in all biological replicates). A matrix including array depletion ratios and the following features were generated for each spacer target: target strand, transcript targeting, ORI targeting, target sequence motifs, flanking sequence motifs, and target secondary structure. The degree to which different features in this matrix explained target depletion for CLUST.195009 systems was investigated.

FIG. 35 shows the degree of interference activity of the engineered composition, with a non-coding sequence, by plotting for a given target the normalized ratio of sequencing reads in the screen output versus the screen input. The results are plotted for each DR transcriptional orientation. In the functional screen for the composition, an active effector complexed with an active RNA guide will interfere with the ability of the pACYC184 to confer E. coli resistance to chloramphenicol and tetracycline, resulting in cell death and depletion of the spacer element within the pool. Comparison of the results of deep sequencing the initial DNA library (screen input) versus the surviving transformed E. coli (screen output) suggests specific target sequences and DR transcriptional orientations that enable an active, programmable CRISPR system. The screen also indicates that the effector complex is only active with one orientation of the DR. As such, the screen indicated that the CLUST.195009 SRR6201554 effector was active in the “forward” orientation (5′-CCAG . . . CGAC-[spacer]-3′) of the DR (FIG. 35).

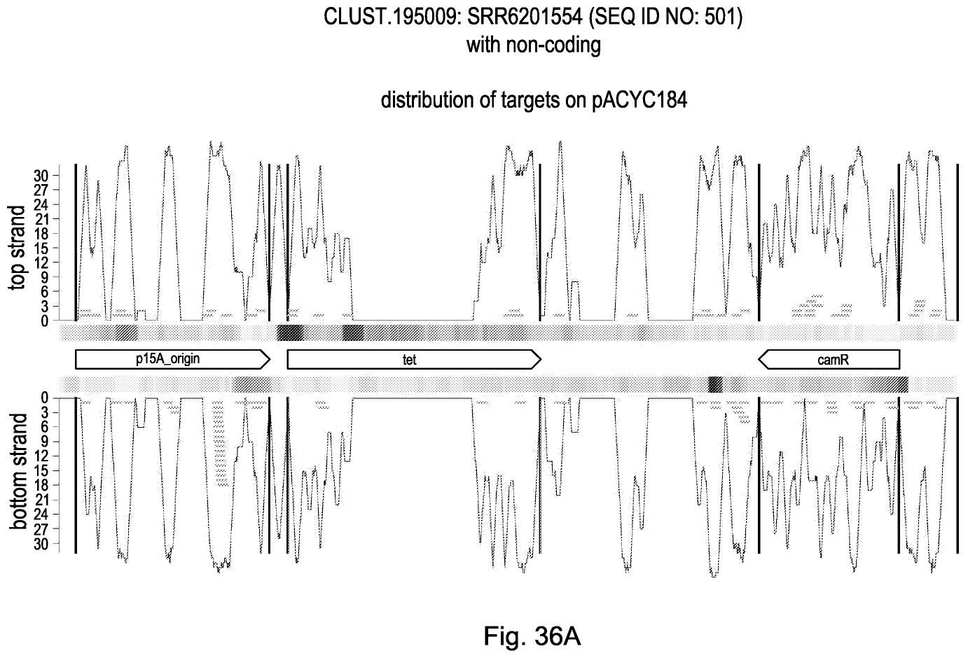

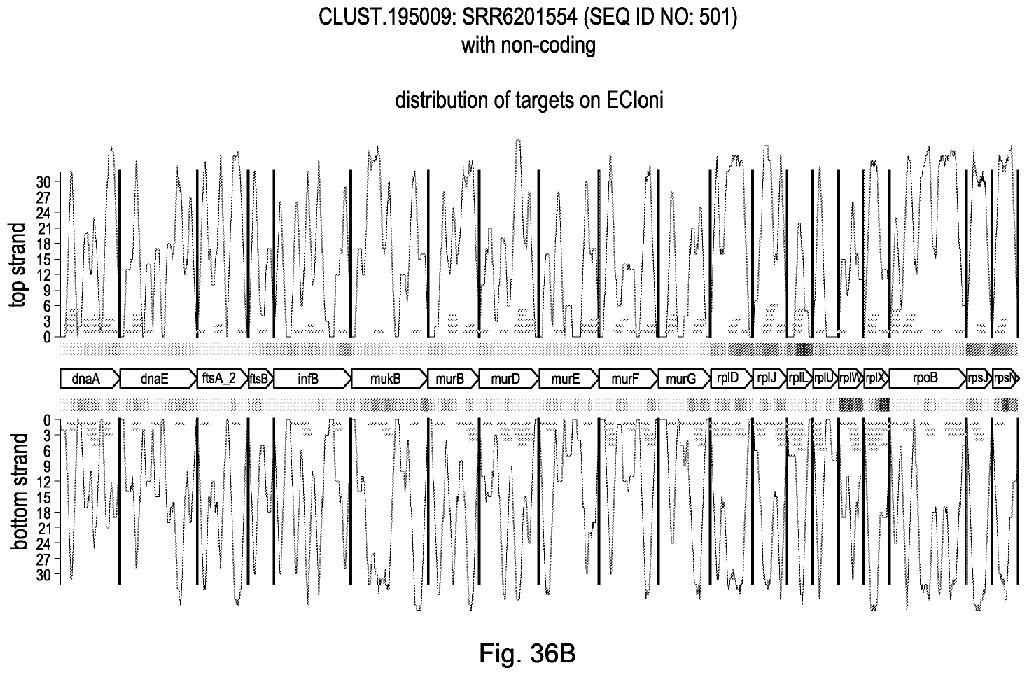

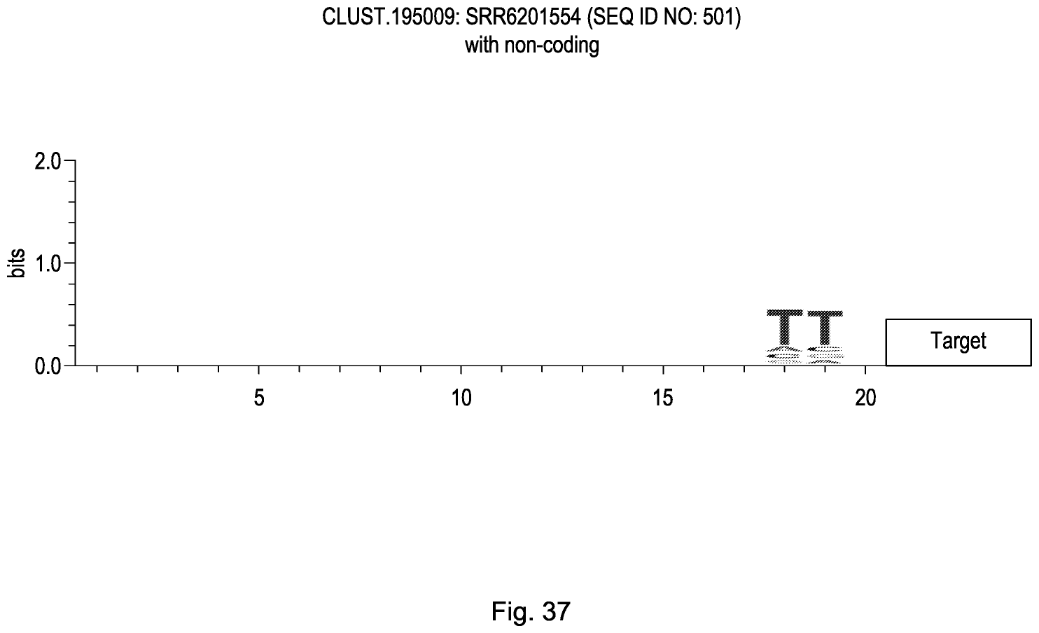

FIG. 36A and FIG. 36B depict the location of strongly depleted targets for the CLUST.195009 SRR6201554 effector (plus non-coding sequence) targeting pACYC184 and E. coli E. Cloni essential genes, respectively. Flanking sequences of depleted targets were analyzed to determine the PAM for CLUST.195009 effectors. A WebLogo representation (Crooks et al., Genome Research 14: 1188-90, 2004) of the PAM sequence for CLUST.195009 SRR6201554 is shown in FIG. 37, where the “20” position corresponds to the nucleotide adjacent to the 5′ end of the target.

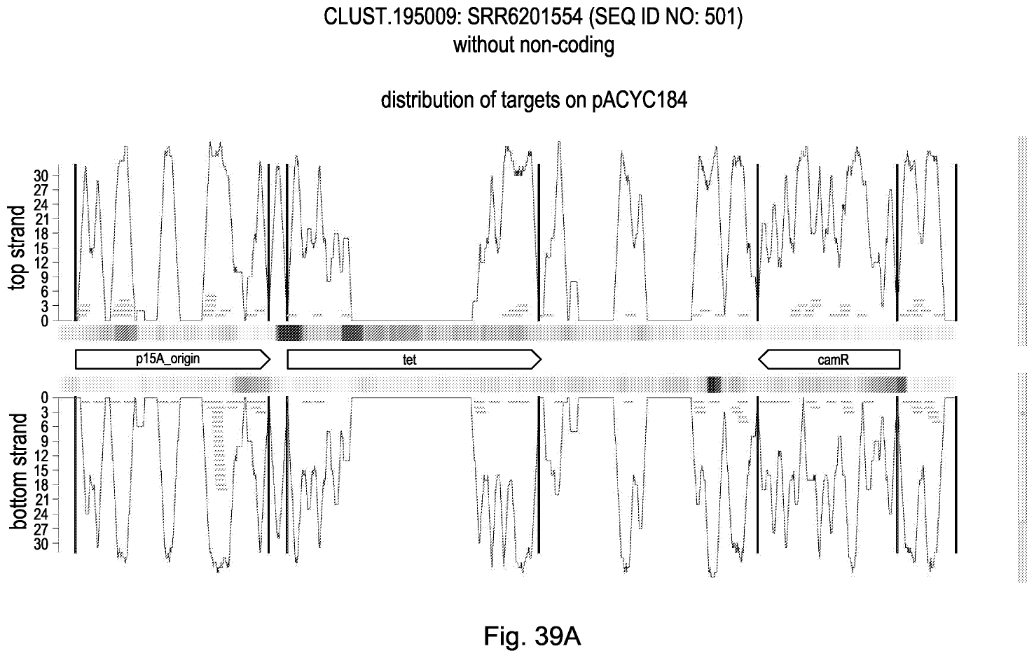

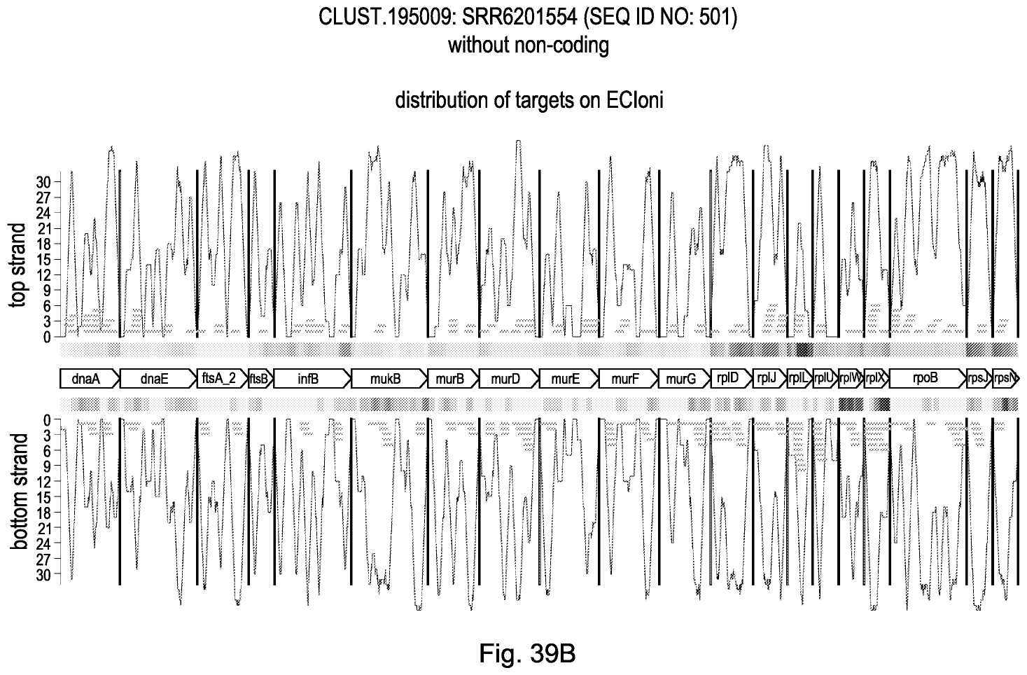

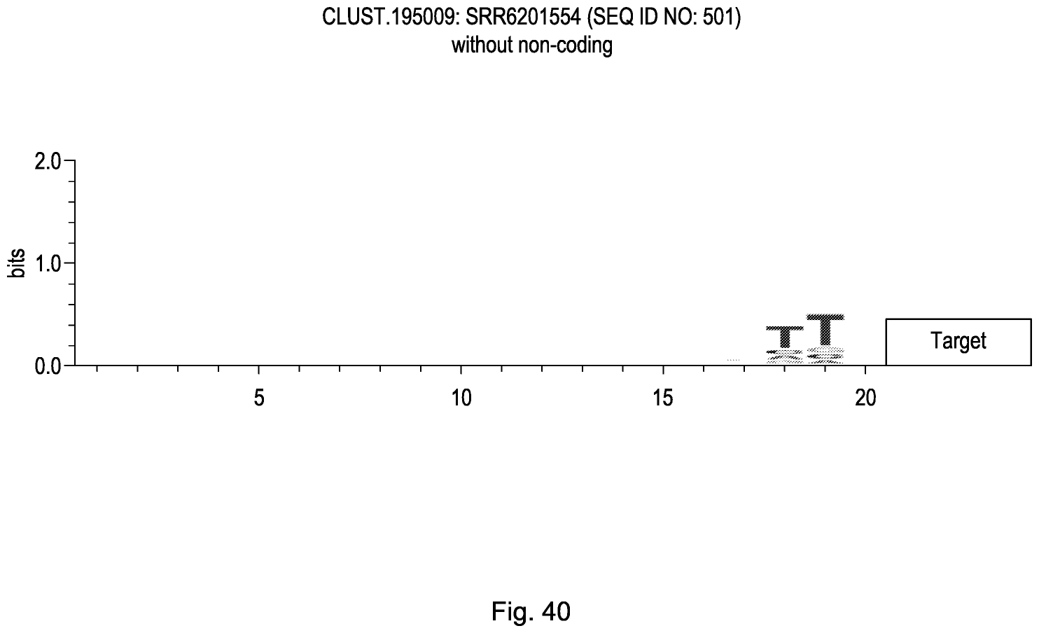

Furthermore, FIG. 38 shows that the CLUST.195009 SRR6201554 effector retains activity in the absence of the non-coding sequence. In agreement with FIG. 35, the CLUST.195009 SRR6201554 effector, without the non-coding sequence, was active in the “forward” orientation (5′-CCAG . . . CGAC-[spacer]-3′) of the DR. FIG. 39A and FIG. 39B depict the locations of the strongly depleted targets for the CLUST.195009 SRR6201554 effector, without the non-coding sequence, targeting pACYC184 and E. coli E. Cloni essential genes, respectively. A WebLogo of the PAM sequence for CLUST.195009 SRR6201554, without the non-coding sequence, is shown in FIG. 40, where the “20” position corresponds to the nucleotide adjacent to the 5′ end of the target.

These results suggest that effectors of CLUST.195009 do not require a tracrRNA. CLUST.195009 effectors may thus be self-processing, allowing for ease in multiplexing.

### Example 13—Identification of Components of CLUST.057059 CRISPR-Cas Systems

This protein family was identified using the computational methods described above. The CLUST.057059 system comprises single effectors associated with CRISPR systems found in particular environments, including freshwater, aquatic, biofilm, crustacean, microbial mat, sediment and soil crust environments as well as in Aphanizomenon phage, Cyanothece sp., Propionimicrobium lymphophilum, Sphaerospermopsis reniformis (TABLE 28). Exemplary CLUST.057059 effectors include those shown in TABLES 28 and 29, below. Examples of direct repeat sequences and spacer lengths for these systems are shown in TABLE 30. Optionally, the system includes a tracrRNA that is contained within a non-coding sequence listed in TABLE 31.

TABLE 288
Representative CLUST.057059 Effector Proteins

| | | | effector | SEQ |
|---|---|---|---|---|
| source | effector accession | # spacers | size | ID NO |
| aquatic-freshwater | 3300023179\|Ga0214923_10000634_60\|P | 10 | 623 | 601 |
| Aphanizomenon phage | ATW59329.1 | 10 | 466 | 602 |
| vB_AphaS-CL131 (MG209611) | | | | |
| aquatic metagenome | SRR3727507_43825_27\|P | 22 | 505 | 603 |
| aquatic metagenome | SRR3727511_164637_6\|M | 3 | 505 | 604 |
| aquatic metagenome | SRR3727516_4364_11\|P | 11 | 505 | 605 |
| aquatic metagenome | SRR3727519_259012_1\|M | 5 | 505 | 606 |
| aquatic metagenome | SRR3727519_13189_1\|M | 11 | 505 | 607 |
| aquatic metagenome | SRR5216639_261560_2\|P | 10 | 560 | 608 |
| aquatic metagenome | SRR5216639_2064603_3\|P | 3 | 522 | 609 |
| aquatic metagenome | SRR5216639_2018995_2\|M | 13 | 553 | 610 |
| aquatic metagenome | SRR5216639_409921_41\|M | 9 | 633 | 611 |
| aquatic metagenome | SRR5216639_409921_40\|P | 9 | 622 | 612 |
| aquatic metagenome | SRR8571171_1127661_43\|P | 9 | 622 | 613 |
| aquatic-freshwater | 3300002370\|release\|scaffold29556_3\|P | 3 | 557 | 614 |
| aquatic-freshwater | 3300002382\|1017448\|scaffold9922858_2\|M | 8 | 506 | 615 |
| aquatic-freshwater | 3300002396\|B570J29629_1000007_3\|P | 3 | 557 | 616 |
| aquatic-freshwater | 3300002396\|B570J29629_1000558_1\|M | 9 | 506 | 617 |
| aquatic-freshwater | 3300002408\|1017274\|scaffold00152_5\|M | 8 | 557 | 618 |
| aquatic-freshwater | 3300013005\|Ga0164292_10021448_2\|P | 15 | 391 | 619 |
| aquatic-freshwater-freshwater | 3300027808\|Ga0209354_10002575_5\|M | 11 | 559 | 620 |
| lake | | | | |
| aquatic-freshwater-freshwater | 3300020048\|Ga0207193_1000230_60\|P | 10 | 557 | 621 |
| lake sediment | | | | |
| aquatic-freshwater-freshwater | 3300015360\|Ga0163144_10269315_2\|M | 4 | 504 | 622 |
| microbial mat | | | | |
| aquatic-freshwater-glacier | 3300022561\|Ga0212090_10036221_2\|M | 6 | 585 | 623 |
| valley | | | | |
| aquatic-freshwater-lake | 3300005805\|Ga0079957_1000538_44\|M | 6 | 507 | 624 |
| aquatic-freshwater-lake | 3300005805\|Ga0079957_1019747_2\|M | 19 | 624 | 625 |
| aquatic-marine | 3300001043\|JGI12316J14372_10004500_6\|M | 4 | 484 | 626 |
| aquatic-marine | 3300001043\|JGI12316J15309_10000277_14\|M | 2 | 484 | 627 |
| aquatic-marine | 3300001341\|1010336\|scaffold_14_1570\|P | 2 | 484 | 628 |
| aquatic-marine | 3300027607\|Ga0207422_1002635_7\|P | 2 | 484 | 629 |
| aquatic-marine-aqueous | 3300007960\|Ga0099850_1030516_1\|P | 2 | 574 | 630 |
| aquatic-thermal springs- | 3300028735\|Ga0272446_1017865_2\|M | 3 | 454 | 631 |
| microbial mat | | | | |
| biofilm metagenome | SRR6869054_1535443_5\|P | 2 | 484 | 632 |
| biofilm metagenome | SRR6869054_457352_2\|P | 7 | 614 | 633 |
| biofilm metagenome | SRR6869055_1317318_5\|P | 2 | 484 | 634 |
| crustacean metagenome | SRR3137750_862387_23\|M | 6 | 553 | 635 |
| crustacean metagenome | SRR3138838_558748_1\|P | 2 | 547 | 636 |
| crustacean metagenome | SRR3139299_296800_1\|M | 2 | 557 | 637 |
| crustacean metagenome | SRR3139690_493772_1\|M | 2 | 550 | 638 |
| (SRR3139690) | | | | |
| crustacean metagenome | SRR3139690_185157_1\|M | 3 | 500 | 639 |
| (SRR3139690) | | | | |
| crustacean metagenome | SRR3139690_493772_1\|P | 2 | 618 | 640 |
| (SRR3139690) | | | | |
| Cyanothece sp. PCC 8801 | WP_015957291.1 | 38 | 494 | 641 |
| (CP001287) | | | | |
| Cyanothece sp. PCC 8801 | CP001287_4134\|P | 38 | 505 | 642 |
| (CP001287) | | | | |
| Cyanothece sp. PCC 8802 | WP_015785111.1 | 11 | 494 | 643 |
| (NC_013161) | | | | |
| Cyanothece sp. PCC 8802 | NC_013161_4122\|P | 11 | 505 | 644 |
| (NC_013161) | | | | |
| freshwater bacterium LH6-2 | OVJI010000022_17\|P | 4 | 649 | 645 |
| Bacteria. (OVJI010000022) | | | | |
| freshwater metagenome | CAAAFE010002307_2\|M | 5 | 515 | 646 |
| (CAAAFE010002307) | | | | |
| freshwater metagenome | OGWG01000020_5\|P | 19 | 660 | 647 |
| (OGWG01000020) | | | | |
| freshwater metagenome | OGWG01000020_4\|M | 19 | 657 | 648 |
| (OGWG01000020) | | | | |
| freshwater metagenome | OGWL01001214_2\|P | 9 | 556 | 649 |
| (OGWL01001214) | | | | |
| freshwater metagenome | OGWO01000325_8\|P | 13 | 560 | 650 |
| (OGWO01000325) | | | | |
| freshwater metagenome | OGWP01000327_5\|M | 7 | 657 | 651 |
| (OGWP01000327) | | | | |
| freshwater metagenome | OGWP01001429_1\|P | 4 | 572 | 652 |
| (OGWP01001429) | | | | |
| freshwater metagenome | OGWP01001429_1\|M | 4 | 581 | 653 |
| (OGWP01001429) | | | | |
| freshwater metagenome | SRR3989431_2283179_3\|M | 11 | 557 | 654 |
| (SRR3989431) | | | | |
| freshwater metagenome | SRR3989431_168870_5\|M | 10 | 558 | 655 |
| (SRR3989431) | | | | |
| freshwater metagenome | SRR4754862_508403_7\|P | 18 | 557 | 656 |
| (SRR4754862) | | | | |
| freshwater metagenome | SRR5468113_274154_3\|M | 2 | 574 | 657 |
| (SRR5468113) | | | | |
| freshwater metagenome | SRR5468149_1084624_1\|P | 3 | 613 | 658 |
| (SRR5468149) | | | | |
| freshwater metagenome | SRR5574091_1575611_4\|M | 13 | 548 | 659 |
| (SRR5574091) | | | | |
| freshwater metagenome | SRR6475630_1852385_2\|M | 2 | 557 | 660 |
| (SRR6475630) | | | | |
| freshwater metagenome | SRR6475633_3251865_1\|M | 12 | 471 | 661 |
| (SRR6475633) | | | | |
| fungi-freshwater | 3300031784\|Ga0315899_10084352_2\|M | 2 | 525 | 662 |
| (3300031784\|Ga0315899_10084352) | | | | |
| fungi-freshwater | 3300031857\|Ga0315909_10035026_2\|M | 5 | 548 | 663 |
| (3300031857\|Ga0315909_10035026) | | | | |
| fungi-freshwater | 3300032092\|Ga0315905_10155087_1\|P | 5 | 550 | 664 |
| (3300032092\|Ga0315905_10155087) | | | | |
| fungi-freshwater | 3300032092\|Ga0315905_10155087_2\|M | 5 | 559 | 665 |
| (3300032092\|Ga0315905_10155087) | | | | |
| lake water metagenome | SRR6476078_1283286_3\|P | 15 | 391 | 666 |
| (SRR6476078) | | | | |
| lake water metagenome | SRR6476078_1283286_3\|M | 15 | 385 | 667 |
| (SRR6476078) | | | | |
| lake water metagenome | SRR7812565_865334_1\|P | 3 | 623 | 668 |
| (SRR7812565) | | | | |
| microbial mat metagenome | SRR6448207_482583_3\|P | 7 | 613 | 669 |
| (SRR6448207) | | | | |
| microbial mat metagenome | SRR7244345_343276_44\|P | 39 | 522 | 670 |
| (SRR7244345) | | | | |
| Propionimicrobium | WP_016456110.1 | 15 | 532 | 671 |
| lymphophilum ACS-093-V- | | | | |
| SCH5 (AGZR01000008) | | | | |
| Propionimicrobium | AGZR01000008_18\|M | 15 | 513 | 672 |
| lymphophilum ACS-093-V- | | | | |
| SCH5 (AGZR01000008) | | | | |
| sediment metagenome | SRR8162779_1368970_53\|P | 5 | 522 | 673 |
| (SRR8162779) | | | | |
| soil crust metagenome | SRR5855413_300079_4\|M | 3 | 524 | 674 |
| (SRR5855413) | | | | |
| soil crust metagenome | SRR5855413_1311040_12\|M | 12 | 516 | 675 |
| (SRR5855413) | | | | |
| soil crust metagenome | SRR5855415_346708_105\|P | 2 | 502 | 676 |
| (SRR5855415) | | | | |
| soil crust metagenome | SRR5855420_1331858_67\|P | 12 | 509 | 677 |
| (SRR5855420) | | | | |
| soil crust metagenome | SRR5855420_1331858_68\|M | 12 | 516 | 678 |
| (SRR5855420) | | | | |
| soil crust metagenome | SRR5855435_639752_6\|P | 3 | 524 | 679 |
| (SRR5855435) | | | | |
| soil metagenome | SRR6201738_878635_2\|M | 4 | 685 | 680 |
| (SRR6201738) | | | | |
| soil metagenome | SRR6201739_286584_1\|P | 3 | 685 | 681 |
| (SRR6201739) | | | | |
| Sphaerospermopsis reniformis | GCL37732.1 | 26 | 626 | 682 |
| (BJCE01000094) | | | | |

TABLE 299
Amino acid Sequences of Representative CLUST.057059 Effector Proteins

>3300023179|Ga0214923_10000634_60|P
[aquatic-freshwater]
MKTIKIKIKLTTDQVQLCDRYLEELTWLWNLTLSNQLHNHCVTWYDWAAKLSANLDKATEKLDKLKPEQQQLIKDYYRT
KDKPKLSKKEQELVAKFDIFARWNSFSLDGIIPVPLRLGNSGYEGLSCQIATGGNYWKRDENINIPINTKKGIVHVKGY
KLVKGDKPWQRIEIVPHKYRTFPGGKFEGRELTTLEKLDNVNGLNTLRAFQNLPDLQVSSHYIGGLLAFFKESWSAFLD
PKRMNSRKPKFKKDSDKITTLSNNQCAPNRIDVNKNIVTVTGFSPIAIIDKNWVKRLNLSEVLPRTYMLTQNPSGYYIN
IVIAHPLHEEKTALVKKLPKVKKEFGEDSQEYEDIKSKIKFLEQQIKEASIVKGKDLSVGIDPGVQAVVSTDHGALFLP
NLTRERVSIHIEELQSRLDNIELINDKKWKSLGNKTPRIKTKNETKLQEKISRLHERGANSSNAFNHKLSTRLSRTYEH
IAWEDTQINNLLKQVEPKALPEGIGYAHNGASAKRGLNWIMRQRCLSDLKAKTKQKTENRGGNFHEPPANYSSQTCHCC
KQKGERRSQHEFICKNSDCKLFDIPQQADTNAARNHKQNGGFELGEVKYHNVKLVYQKPKRFKKKRLTNQ
(SEQ ID NO: 601)
>ATW59329.1
[Aphanizomenon phage vB_AphaS-CL131]
MKTKTIEFKIYPTLAQSQTIDKWLQDIKWVWNKGLSLKFADRQKHYRTQIGDRNIPDGLPLRWKWRKVEATDKKGKITE
KWEKIRLVGGGVVRPKSGYPYCPIREHRNIEDPGKFKYFRNDNSPQFVIDIPSEFKEGMADTLKKAWQAYSDPKRPTQK
PKFKGKQDKVRSLTSLHAGGKSKLLKPERIPGSDNGFVQFPKLGKLKVKGLYSRHDWVEWGSAKIVKEPSGYYLHVCVD
VPNDPLPKSDKSVGIDPGLLSVITTDQGREVAPPKLFRKQQTKLRRLQRKASRQQKGGSNQKKTYQKVALQHEKIRRSR
NAFNHKLSTKVVREYSGIVMEDLKIQNLSRKPKAKKREDGNGYEQNGAKRKAGLNKSFADSALGDLISKIETKCKDTDR
EFVKVAAHHTTVDCSNCGAKIKKALSQRTHRCTECGYEDGRDSNAAKNILIKGQKEFKTVYRAWAWEHGET
(SEQ ID NO: 602)
>SRR3727507_43825_27|P
[aquatic metagenome]
MKTLEFKLTLNQSQRAKLNDWMSANKYVWNEGVRHIEWFNEFNRYHKPDKKSYPCSPIRSQNFWKKYRGQEPIENYGLG
EFPSCPIGWINDSFNPELPSFPVSRSPEHEQIHNISLFSFIKLFAHSRNQDKPWLLEVPSKFINGEVDCLINSWNAFVK
SPANLKRRPPKYKGKHDKIDALIHNNSKGMGTKGDSINVPKLGYVRVKGFSDRWYFEDGKPIDFCPLKICKKASGWYLL
ITGNTPDRHYQKSDSIAGFDLGVHEILTDEKNYHATSPRYLKNSQASLKRLQRKMSRQFRMNEGDKTWERKNWNRTKLK
LARTHERIARQRRALNHFLSHKVVNTHAVIALEDLKLLNMTKAVKTGEAGVQNGRKSKSGLNRAMLDNGLGQLVGMIET
KAKVADRIVVQVDPKHTSQECPHCGHIEKKANKKNREVTCGNCGASFHRDTSAAEIIKRRGIARLEGGNVESKAKKERK
AKYGKRSKVVESAIDPRTIFVEALQDLPLFG
(SEQ ID NO: 603)
>SRR3727511_164637_6|M
[aquatic metagenome]
MKTLEFKLTLNQSQRAKLNDWMSANKYVWNEGVRHIEWFNEFNRYHKPDKKSYPCSPIRSQNFWKKYRGQEPIENYGLG
EFPSCPIGWINDSFNPELPSFPVSRSPEHEQIHNISLFSFIKLFAHSRNQDKPWLLEVPSKFINGEVDCLINSWNAFVK
SPANLKRRPPKYKGKHDKIDALIHNNSKGMGTKGDSINVPKLGYVRVKGFSDRWYFEDGKPIDFCPLKICKKASGWYLL
ITGNTPDRHYQKSDSIAGFDLGVHEILTDEKNYHATSPRYLKNSQASLKRLQRKMSRQFRMNEGDKTWERKNWNRTKLK
LARTHERIARQRRALNHFLSHKVVNTHAVIALEDLKLLNMTKAVKTGEAGVQNGRKSKSGLNRAMLDNGLGQLVGMIET
KAKVADRIVVQVDPKHTSQECPHCGHIEKKANKKNREVTCGNCGASFHRDTSAAEIIKRRGIARLEGGNVESKAKKERK
AKYGKRSKVVESAIDPRTIFVEALQDLPLFG
(SEQ ID NO: 604)
>SRR3727516_4364_11|P
[aquatic metagenome]
MKTLEFKLTLNQSQRAKLNDWMSANKYVWNEGVRHIEWFNEFNRYHKPDKKSYPCSPIRSQNFWKKYRGQEPIENYGLG
EFPSCPIGWINDSFNPELPSFPVSRSPEHEQIHNISLFSFIKLFAHSRNQDKPWLLEVPSKFINGEVDCLINSWNAFVK
SPANLKRRPPKYKGKHDKIDALIHNNSKGMGTKGDSINVPKLGYVRVKGFSDRWYFEDGKPIDFCPLKICKKASGWYLL
ITGNIPDRHYQKSDSIAGFDLGVHEILTDEKNYHATSPRYLKNSQASLKRLQRKMSRQFRMNEGDKTWERKNWNRTKLK
LARTHERIARQRRALNHFLSHKVVNTHAVIALEDLKLLNMTKAVKTGEAGVQNGRKSKSGLNRAMLDNGLGQLVGMIET
KAKVADRIVVQVDPKHTSQECPHCGHIEKKANKKNREVTCGNCGASFHRDTSAAEIIKRRGIARLEGGNVESKAKKERK
AKYGKRSKVVESAIDPRTIFVEALQDLPLFG
(SEQ ID NO: 605)
>SRR3727519_259012_1|M
[aquatic metagenome]
MKTLEFKLTLNQSQRAKLNDWMSANKYVWNEGVRHIEWFNEFNRYHKPDKKSYPCSPIRSQNFWKKYRGQEPIENYGLG
EFPSCPIGWINDSFNPELPSFPVSRSPEHEQIHNISLFSFIKLFAHSRNQDKPWLLEVPSKFINGEVDCLINSWNAFVK
SPANLKRRPPKYKGKHDKIDALIHNNSKGMGTKGDSINVPKLGYVRVKGFSDRWYFEDGKPIDFCPLKICKKASGWYLL
ITGNTPDRHYQKSDSIAGFDLGVHEILTDEKNYHATSPRYLKNSQASLKRLQRKMSRQFRMNEGDKTWERKNWNRTKLK
LARTHERIARQRRALNHFLSHKVVNTHAVIALEDLKLLNMTKAVKTGEAGVQNGRKSKSGLNRAMLDNGLGQLVGMIET
KAKVADRIVVQVDPKHTSQECPHCGHIEKKANKKNREVTCGNCGASFHRDTSAAEIIKRRGIARLEGGNVESKAKKERK
AKYGKRSKVVESAIDPRTIFVEALQDLPLFG
(SEQ ID NO: 606)
>SRR3727519_13189_1|M
[aquatic metagenome]
MKTLEFKLTLNQSQRAKLNDWMSANKYVWNEGVRHIEWFNEFNRYHKPDKKSYPCSPIRSQNFWKKYRGQEPIENYGLG
EFPSCPIGWINDSFNPELPSFPVSRSPEHEQIHNISLFSFIKLFAHSRNQDKPWLLEVPSKFINGEVDCLINSWNAFVK
SPANLKRRPPKYKGKHDKIDALIHNNSKGMGTKGDSINVPKLGYVRVKGFSDRWYFEDGKPIDFCPLKICKKASGWYLL
ITGNTPDRHYQKSDSIAGFDLGVHEILTDEKNYHATSPRYLKNSQASLKRLQRKMSRQFRMNEGDKTWERKNWNRTKLK
LARTHERIARQRRALNHFLSHKVVNTHAVIALEDLKLLNMTKAVKTGEAGVQNGRKSKSGLNRAMLDNGLGQLVGMIET
KAKVADRIVVQVDPKHTSQECPHCGHIEKKANKKNREVTCGNCGASFHRDTSAAEIIKRRGIARLEGGNVESKAKKERK
AKYGKRSKVVESAIDPRTIFVEALQDLPLFG
(SEQ ID NO: 607)
>SRR5216639_261560_2|P
[aquatic metagenome]
MSNVSENVRTKEFKLILTPEQERTLEDWMLVCKWVWNRSLGLIEEFNDWNPYDKISKSYVPAMPLQRYDRKLKQFVRVE
IPDWKLSTERKETSKGLVHPVSIDKDTPQIDSLNSKKSILYGFLKVFGHQYHKDRIITYMVRGQERKVKFTDCPAKFIQ
GVSHELSKAWDGFLAGRHSRPRFKTARDKVMTLLHYNAKDIGVKGSSINIPKLGYVEVIGFDNRWYGVDFNPMKICKKA
SGWYVQFTATVPVKKFKKTGLACGIDPGHQFVMALDNGHTIKAAQPLKQNLRRLKRMQKRLSRKFRINNGKTKNWEKLN
QRIAKLHEKIARHRRSFNHWHSTNLVKWFDVIFVEDYKPANVYRKAKAKTKTDEQGNPMTADNGAVVYEKNNQKRKKGS
NRTGSDIAIGQAIELLETKAKEHDKLVIRVPAYGTTLRCANCGHEERKSLSQRIHHCANCGYTVARDVNSGQNVKLKGL
AQLALDHGVELVDNFWYQILKNATTSSCSEKKGKKKAKKKDVVSIPDDLFKNGVSAFLIAETVKTRAIIKQKYLENLNK
TAKTLMG
(SEQ ID NO: 608)
>SRR5216639_2064603_3|P
[aquatic metagenome]
MDPSIKVRKVEFKVPLDTQQRQIFEHHSQALRFIWNTGLACIQWREWWEKWQQVLESPEYQGYAPEPVEMDWGYNLIGK
KKVYGLCCDRVRFRIEKEDPVLDVGEWIDGTKLGSGKSIAGYWVAYPCKPDSVREHWTEQPRLPLLMKNPYMALRSMFA
KKNWDPDSVEGKIIHSLNTAWVAGELKALGDAWSSYKSGVRKRPRFKKRHDRSNLINPSSKQTKFCKNSLTLPGMGRVT
VKGLQKRWGNKPACPISLIKEGDEFYLQLSGGVEVPATRSNGVTIGIDPGSVRLATDDSGRAIDAPKFGKRAMRRAQKL
QAAINRKLRLNSDRVYDDADPSKFYLKPRRDWKRKNLQKVRTKLQRLSAKTARQRRAFNHFHSTRLVQSADVIIMEGIE
LKNVTRAVKKGKSGEHNKRKAKAGLNRSLLDNAIGQFYGMVEQKAKDAGKVTARVNPFRTSMTCHQCGFSDAKNRKTQA
VFHCQKCGHHDNADRNAAANIKRMGILGITKLTFDNEWNITVPDWDKI
(SEQ ID NO: 609)
>SRR5216639_2018995_2|M
[aquatic metagenome]
MNTITKTRKVEFKVTLDTQQRQVFDHHSHALRFVWNTGLACIQWREWWEKWQQVLESPDRVGYAPEPVEMEWAYNLVGK
KKVYGLACDRVRFRLEKSDPTLANGEWVANGKKEGGYWVAYPCKPQPVREHWTEEPRLMLLMKNPYMALRTMFAKKNWD
PDSVQGKIIHSLNTAWVAGELKALGDAWSSYKSGVRKRPRFKKRHDRSNLINPSSKQTKFSGNSLTLPGMGRVTVKGLQ
QRWGDKPACPIALIKEGDEFYLQLTGEVQVPAVRSNGVTIGLDPGSVRLATADNGSAIEAPKFGRNIMRRNKKLQASIN
RKLRLNSDRVHDEADPSKFYLKPRKNWKRKNLQKVRTKLQRLNTKAARQRRAYNHFHSTRIVQSADVIIMEGLELKNVT
RAVKKGKSGQHNKRKAKAGLNRSLLDNAIGQFYGMVEQKAKDAGKVTTRVNPFRTSMTCHQCGFSDAKNRTTQAVFHCQ
KCGHRDNADRNAALNIKRMGVLGVTKLTFDNDWNITIEGWGSVAALHSKGLKPVGTDKKRSKRVQKEHPSQMVLELGAP
(SEQ ID NO: 610)
>SRR5216639_409921_41|M
[aquatic metagenome]
MTRENKQAVNNMKTIKIKVKLTPSQIESCNKYLEELSWLWNLVLANQLHNHCIRWYEWAAKLSAELAKTNEKLEKLKPE
QQQLIKDYYNSTTKPKLSKKDQELVNKFNILSRWSPFDLEGIIPVPLRIGNSAYEGLSCQIATGGSYWKRDDNTVIPVN
TKKGIAFIKGSKLVKGDRPWQRIEIKPHEYRKFPGGKYEGRELVTLEKFDNLNGLNTLRASQNLPDLTVSSHYIGGLLG
FFKESWKAFLDSKRTISRKPKFKKDNDKIATLSNSQCPPNRIDVDKNIIAITGFSAVKVDDKSWVKRLNLSQSLPRTYM
LTKQQSGYYINIIVAHPLQEEKSILANKLPKVKKQFGEDSQEYADIKSKLKFIEQQIKESSVIEGKGLSVGIDPGIQSV
VSTDHGALFLPNLSRERISIHIEELQSKLDNAESINDKKWKEAGNKTPRPRTNAEIKLQDKIRRLHERGSNSSNAFNHK
LSTRLSRTYEHIAWENTQIKNLLKQVEPKSLPEGTGYAHNGASAKRGLNWIMRQRCLGDLEKKTKLKVENRGGSFQQPP
ANYTSQTCHQCKQKGERLSQHEFICKNSECRLFNVPQQADTNAARNHKQNAGFELGEVKYNSVRLTYEKPQRFKKKRAD
N
(SEQ ID NO: 611)
>SRR5216639_409921_40|P
[aquatic metagenome]
MKTIKIKVKLTPSQIESCNKYLEELSWLWNLVLANQLHNHCIRWYEWAAKLSAELAKTNEKLEKLKPEQQQLIKDYYNS
TTKPKLSKKDQELVNKFNILSRWSPFDLEGIIPVPLRIGNSAYEGLSCQIATGGSYWKRDDNTVIPVNTKKGIAFIKGS
KLVKGDRPWQRIEIKPHEYRKFPGGKYEGRELVTLEKFDNLNGLNTLRASQNLPDLTVSSHYIGGLLGFFKESWKAFLD
SKRTISRKPKFKKDNDKIATLSNSQCPPNRIDVDKNIIAITGFSAVKVDDKSWVKRLNLSQSLPRTYMLTKQQSGYYIN
IIVAHPLQEEKSILANKLPKVKKQFGEDSQEYADIKSKLKFIEQQIKESSVIEGKGLSVGIDPGIQSVVSTDHGALFLP
NLSRERISIHIEELQSKLDNAESINDKKWKEAGNKTPRPRTNAEIKLQDKIRRLHERGSNSSNAFNHKLSTRLSRTYEH
IAWENTQIKNLLKQVEPKSLPEGTGYAHNGASAKRGLNWIMRQRCLGDLEKKTKLKVENRGGSFQQPPANYTSQTCHQC
KQKGERLSQHEFICKNSECRLFNVPQQADTNAARNHKQNAGFELGEVKYNSVRLTYEKPQRFKKKRADN
(SEQ ID NO: 612)
>SRR8571171_1127661_43|P
[aquatic metagenome]
MKTIKIKVKLTPSQIESCDKYLEELSWLWNLVLANQLHNHCIRWYEWAAKLSAELAKTNEKLEKLKPEQQQLIRDYYNS
TTKPKLSKKDQELVNKFNILSRWSPFDLEGIIPVPLRIGNSAYEGLSCQIATGGSYWKRDDNTVIPVNTKKGISFIKGS
KLVKGDKPWQRVEIKPHEYRKFPGGKYEGRELVTLEKFDNLNGLNTLRASQNLPDLTVSSHYIGGLLGFFKESWKAFLD
SKRTISRKPKFKKDNDKIATLSNSQCPPNRIDVDKNIIAITGFSAVKVDDKSWVKRLNLSQSLPRTYMLTKQQSGYYIN
IIVAHPLQEEKSILANKLPKVKKQFGEDSQEYADIKSKLKFIEQQIKESSVIEGKGLSVGIDPGIQSVVSTDHGALFLP
NLSRERVSIHIEELQSKLDNAESINDKKWKEAGNKTSRPRTNTEIKLQDKIRRLHERGSNSSNAFNHKLSTRLSRTYEH
IAWENTQIKNLLKQVEPKSLPEGTGYAHNGASAKRGLNWIMRQRCLGDLEKKTKLKVENRGGSFQQPPANYTSQTCHQC
KQKGERLSQHEFICKNSECRLFNVPQQADTNAARNHKQNAGFELGEVKY
NGVRLTYEKPQRFKKKRADN
(SEQ ID NO: 613)
>3300002370|release|scaffold29556_3|P
[aquatic-freshwater]
MSNFSENVRTKEFKLILSSEQERTLEDWMLVCKWVWNRSLGLIEEFNEWNPYDKLSKSNVPATPLQRYDRKLKQWVRIE
IPDWKMGIERVEKKRGLIHPVAIDENSPIIDSLDSKKSVLYGFLKVFGHQHHKDRIVTYLVRGEERKVNFTDCPAKFIQ
GVAHELSKAWEGFLAGRHSRPRFKTAKDKVMTLIHYNAKDLGVKDSKVNIPKLGYIEVIGFDKRWYGCDFNPMKICKKA
SGWYIQLTASVPVKQAKKTGLCCGIDPGHQFVMALDNGHTIEAAQPLKRSLKRLRKMQQQLSRKYRMNEGKTKNWEKLN
NKIAKLHEKIARHRRSFNHWHSTNLINWFDVIFVEDYKPANVYRKAKAKAKLDTEGNPVVAENGTVIYDKNSQKRKRGS
NRTGSDVAIGQAIDLLETKAKEHGKLVIRVDNWGTTLCCAKCGHQEKKKLSQRTHKCSNCGYTVARDVNSGQNIKLKGL
AQMAINQGVELSDNFWYKFLINKNSATPSCSKKATKKKTKKSDTITIPDDLFKNGVHDFLVAETAKTHAIINRMSLGDF
DRNA
(SEQ ID NO: 614)
>3300002382|1017448|scaffold9922858_2|M
[aquatic-freshwater]
MGNSGYEGLSCQIATGGNYWKRDENINIPINTKKGIIHVKGYKLVKGDKLWQRIEIVPHKYRTFPGGKFEGRELTTLEK
LDNVNGLNTLRAFQNLPDLQVSSHYIGGLLAFFKESWSAFLDPKRMNSRKPKFKKDSDKITTLSNNQCAPNRIDVNKNI
VTVTGFSPITIIDKNWVKRLNLSQVLPRTYMLTQNPSGYYINIVIAHPLHEEKIALVKKLPKVKKEFGEDSQEYEDIKS
KIKFLEQQIKESSIVKGKDLSVGIDPGVQAVVSTDHGALFLPNLTRERVSIHIEELQSRLDNAELINDKKWKSLGNKTP
RIKTKNETKLQEKISRLHERGANSSNAFHKLSTRLSRTYEHIAWEDTQINNLLKQVEPKALPEGVGYAHNGASAKRGNL
NWIMRQRCLSDLKAKTKQKTENRGGNFHEPPANYSSQTCHCCGQKGERRSQHEFVCKNSDCKLFDIPQQADTNAARNHK
QNGGFELGEVKYHNVKLVYQKPKRFKKKRLTK
(SEQ ID NO: 615)
>3300002396|B570J29629_1000007_3|P
[aquatic-freshwater]
MSNFSENVRTKEFKLILSSEQERTIEDWMLVCKWVWNRSLGLIEEFNEWNPYDKLSKSNVPATPLQRYDRKLKQWVRIE
IPDWKMGIERVEKKRGLIHPVAIDENSPIIDSLDSKKSVLYGFLKVFGHQHHKDRIVTYLVRGEERKVNFTDCPAKFIQ
GVAHELSKAWEGFLAGRHSRPRFKTAKDKVMTLIHYNAKDLGVKDSKVNIPKLGYIEVIGFDKRWYGCDFNPMKICKKA
SGWYIQLTASVPVKQAKKTGLCCGIDPGHQFVMALDNGHTIEAAQPLKRSLKRLRKMQQQLSRKYRMNEGKTKNWEKLN
NKIAKLHEKIARHRRSFNHWHSTNLINWFDVIFVEDYKPANVYRKAKAKAKLDTEGNPVVAENGTVIYDKNSQKRKRGS
NRTGSDVAIGQAIDLLETKAKEHGKLVIRVDNWGTTLCCAKCGHQEKKKLSQRTHKCSNCGYTVARDVNSGQNIKLKGL
AQMAINQGVELSDNFWYKFLINKNSATPSCSKKATKKKTKKSDTITIPDDLFKNGVHDFLVAETAKTHAIINRMSLGDF
DRNA
(SEQ ID NO: 616)
>33000023961B570J29629_1000558_1|M
[aquatic-freshwater]
MGNSGYEGLSCQIATGGNYWKRDENINIPINTKKGIIHVKGYKLVKGDKLWQRIEIVPHKYRTFPGGKFEGRELTTLEK
LDNVNGLNTLRAFQNLPDLQVSSHYIGGLLAFFKESWSAFLDPKRMNSRKPKFKKDSDKITTLSNNQCAPNRIDVNKNI
VTVTGFSPITIIDKNWVKRLNLSQVLPRTYMLTQNPSGYYINIVIAHPLHEEKIALVKKLPKVKKEFGEDSQEYEDIKS
KIKFLEQQIKESSIVKGKDLSVGIDPGVQAVVSTDHGALFLPNLTRERVSIHIEELQSRLDNAELINDKKWKSLGNKTP
RIKTKNETKLQEKISRLHERGANSSNAFNHKLSTRLSRTYEHIAWEDTQINNLLKQVEPKALPEGVGYAHNGASAKRGL
NWIMRQRCLSDLKAKTKQKTENRGGNFHEPPANYSSQTCHCCGQKGERRSQHEFVCKNSDCKLFDIPQQADTNAARNHK
QNGGFELGEVKYHNVKLVYQKPKRFKKKRLTK
(SEQ ID NO: 617)
>3300002408|1017274|scaffold00152_5|M
[aquatic-freshwater]
MSNFSENVRTKEFKLILSSEQERTLEDWMLVCKWVWNRSLGLIEEFNEWNPYDKLSKSNVPATPLQRYDRKLKQWVRIE
IPDWKMGIERVEKKRGLIHPVAIDENSPIIDSLDSKKSVLYGFLKVFGHQHHKDRIVTYLVRGEERKVNFTDCPAKFIQ
GVAHELSKAWEGFLAGRHSRPRFKTAKDKVMTLIHYNAKDLGVKDSKINIPKLGYIEVIGFDKRWYGCDFNPMKICKKA
SGWYIQLTASVPIKQAKKTGLCCGIDPGHQFVMALDNGHTIEAAQPLKRSLKRLRKMQQQLSRKYRMNEGKTKNWEKLN
NKIAKLHEKIARHRRSFNHWHSTNLINWFDVIFVEDYKPANVYRKAKAKAKLDTEGNPVVAENGTVIYDKNSQKRKRGS
NRTGSDVAIGQAIDLLETKAKEHGKLVIRVDNWGTTLCCAKCGHQEKKKLSQRTHKCSNCGYTVARDVNSGQNIKLKGL
AQMAINQGVELSDNFWYKFLINKNSATPSCSKKATKKKTKKSDTITIPDDLFKNGVHDF
LVAETAKTHAIINRMSLGDFDRNA
(SEQ ID NO: 618)
>3300013005|Ga0164292_10021448_2|P
[aquatic-freshwater]
MIFEPNVKVLEFKIHPTEEQVSKIDQSLAACKLLWNLSIALKEESKQRYYRKKHKFDEFSPEIWELSYSGHYDEKEFKS
LKDKEKKLLIGNPCCKIAYFKKTSNGKEYTPLDAIPIRRFMNAENIDKDAVNYLNRKKLAFYFRENTAKFIGEIETEFK
KGFFKSVIKPAYDAAKKGIRGIPRFKGRRDKVETLVNGQPGTIKIKSNGVIVSSKIGLLKVRGLDRLQGKAPRMAKITR
KATGYYLQLTVETDDTIYKESDKCVGLDMGAVAIFTDDLGRQSEAKRYAKIQKKRLDRLQRQASRQKDGSNNQRKTYAK
LARVHEKIARQRKGRNAQLAHKITSEYQSVILEDLKLKNMTAAAKPKEREDGDGYKQNGKKRKSGLNKALLEMSV
(SEQ ID NO: 619)
>3300027808|Ga0209354_10002575_5|M
[aquatic-freshwater-freshwater lake]
MIQANIEDKMTQENITRVIKFRLTLTEEQRLLIDEKMRLLKYLWNLAVSLQEEVNQRRLRDKFGFKEFSPEFWELSYLK
LYSGFSHIEKLPKDKLRLLTGNPCCKIAYLKNKKEYVSLKLEERKKRPYLGNPDAKKHTTKPPDKKDLKKYFYPDLADK
IFEIGSEFRGCFYRYVISKAFETAKKGIRGFPKYKGKLDSVESLIVGHPTLIKIRDGEIFLNKQIGLLVIDDINRLPKD
ADVRMASIVRKASGYYLHLTVNLPVPMHLPSDRQIGIDVGCVNLYTDSEGRHIETKAYYRNKERKIAHLQRKMARQEKG
SANYLKSKEKLALLHEQIKDRRAGYLHQVSHKLTQKYSHIAMEDLKLKNMTKRANVKENEDGTYAHNGASAKSGLNKSI
LDQGIGELKRQLLYKSQEKGGQVVLVNPKFTSQTCFECGHKHKDNRKSQSEFICVSCGHSANADENAAKNILTLSLISS
TIGVLRSSLFSYPSLLGKAVSLLAYVCGSPKGTCEAERERSDPPIDPPAKGGNCLDNFSTLTIKDPRHQLEKASQPIDS
VQLTLF
(SEQ ID NO: 620)
>3300020048|Ga0207193_1000230_60|P
[aquatic-freshwater-freshwater lake sediment]
MSNFSENVRTKEFKLILSSEQERTLEDWMLVCKWVWNRSLGLIEEFNEWNPYDKLSKSNVPATPLQRYDRKLKQWVRIE
IPDWKMGIERVEKKRGLIHPVAIDENSPIIDSLDSKKSVLYGFLKVFGHQHHKDRIVTYLVRGEERKVNFTDCPAKFIQ
GVAHELSKAWEGFLAGRHSRPRFKTAKDKVMTLIHYNAKDLGVKDSKINIPKLGYIEVIGFDKRWYGCDFNPMKICKKA
SGWYVQLTASVPVKQAKKTGLCCGIDPGHQFVMALDNGHTIEAAQPLKRSLKRLRKMQQQLSRKYRMNEGKTKNWEKLN
NKIAKLHEKIARHRRSFNHWHSTNLINWFDVIFVEDYKPANVYRKAKAKAKLDTEGNPVVAENGTVIYDKNNQKRKRGS
NRTGSDVAIGQAIDLLETKAKEHGKLVIRVDNWGTTLCCAKCGHQEKKKLSQRTHKCSNCGYTVARDVNSGQNIKLKGL
AQMAINQGVELSDNFWYKFLINKNSATPSSSKKATKKKTKKSDTITIPDDLFKNGVHDFLVAETAKTHAIINRMSLGDF
DRNA
(SEQ ID NO: 621)
>3300015360|Ga0163144_10269315_2|M
[aquatic-freshwater-freshwater microbial mat]
MKTAEFKLNLNNAQKAELNDWMSALKWVWNEGVRMIDYFNQFNAYHKPDKRSYPCSPILGVNHKRRYKKGFNGCTKSGD
RPMGIYPSCPIGWIADSFNPEEPAYAVSQKADSIQVKNLSFFSLQKLFTHSRNQDRPWLISVPSKFIDGETQSLVNSWE
AFKSGVANRKPPKYKGKRDKIESLIHNNSKGMGVKGDSINIPKIGYVRVKGFSDRWLFEDGKPIDFCPMKICKKASGWY
LQITGHLPDRKYKKTSSIAGFDVGVHEIVTDDIALSVKSPRWLKTNGAKLKKLQRKVSRQWRMNENNDAWERKNWQKTQ
NKIARLHEKIARQRRAFNHYLSHKIVNTHQVIALEDLKLLNMTKAVKTGEAGVANGRKAKSGLNRAMLDVGIGQLVSMI
ETKAKVAGRDVVFVDPKHTSQICPECGHVEKKKNRKNRNVKCSNCGAEFHRDRSAAIEIKKRAVTKMSIPKEVKKERKP
RGVGAFAAKKKAKDSRPIFDPSLIDLPLFG
(SEQ ID NO: 622)
>3300022561|Ga0212090_10036221_2|M
[aquatic-freshwater-glacier valley]
MLTLEFSVQLTEEQKTIAGEWMDSLKHQWNLGLSALEEFDRFSWYNKHDKKPALCCPIAEGEWIKEEKRVASTCSLIRD
PAPKLTSSALQTGGDSLGTMARSDRLPEFLQQVPSKFRMGMLALLSVSWDAYIKDRKNKGKPKYKRRRDRWDVLYSGNL
KDGLSCNGDILKGIPKLGTVVVPKLNQRWKTPTGETPPICAFKLLRDGDKFKVQLTGELQRSYKAKPSDLAVGIDLGFV
YSHILSDGERSPLLSKSESKLENRKQLLQSQLDAKIDQRLTLWLHHPDTGFEQCRELIRVSVETWEKIRECRVAGEVAQ
IIGQKRDKRYMSLRHKLPKSKAEKQLRHQIKNLDRKLADTRKSKDEKLATRLVKKFGHVAIENGLQRQELRERPDPVPN
ESNGHDPNGANAQSEVNKQLKALAPGRKISIIEQRAKRYGRSFVKAESQFTTQECPVCGSLNQPSLKIEETGDRAYHCN
NCGWHCDQDLNAAINIELRSFAFVPQVVLTLMAERARVMSYQKEGDGKIPVNPSWRGKRSAIEGETDSRTKKSKGSSDS
GGRKGKEGAKLKAASRKSQSKAIPKKGSEVLQ
(SEQ ID NO: 623)
>3300005805|Ga0079957_1000538_44|M
[aquatic-freshwater-lake]
MKTLEFKLTLNRSQKARLNDWMTANKWVWNEAVRHIEWFNEFNKYHKPDKKNYPCSPIRSQNFWKKFRGQEPLENYGLG
EFPSCPIGWINDSFDPELPSFPVSRSPKHEQIHNISLFSFIKLFAHSRNQDKPWLLEVPSKFINGEVDCLINSWNAFVK
SPANLKRRPPKYKGKHDKIDALIHNNSKGIGTKGDSINIPKLGYVRVKGFTDRWLFADGKPIDFCPLKICKKASGWYLQ
ISGEVPDRKYRKSESMAGFDLGVHEILTDEKNYHATSPRYLKTSQDKLKRLQRKMSRQFRMNEKDKTWERKNWDKTKIK
LAKAHEKVARQRRTLNHYLSHKAVNTHAVIALEDLKLLNMTKSVKTGEAGVQNGRKAKAGLNRAMLDNGLGQLVGMIET
KAKVADRIVVKVDPKNTSQECPHCGHIEKKANKKNRYVTCSNCGASFHRDTSAAEIIKRRGIARLDGSDTEPKVKKERK
PKYGKRSKPVEPVEAPRPIVDKDLQHLPLFCCA
(SEQ ID NO: 624)
>3300005805|Ga0079957_1019747_2|M
[aquatic-freshwater-lake]
MKTIKIKIKLSSEQAAVCDRYLDELTWLWNLALSNQLHNHCVDWYAWAAKRSADLTKATEKLEKLKPEQQQLVKDYYST
KDKPKLSKKEQELVSKKEFEIFTRWSPFSLEGIIPVPLRLGNSAYEGLSCQIATGGNYWKRDENINIPVNTKKGVIYVK
GYKLVKGDKPWQRIEIVPHKYRTFPSGKYEGRELTTLEKLDNVTGLNTLRAFQNLPDLQVSSHYIGGLLAFFKESWKAF
LDPKRINSRKPKFKKDVDKIATLSNNQCAPNRIDIDKNIVTVTGLTPITIADKNWVKRLNLSQVLPRTYMITKQQSGYY
INIVVAHPLQEEKSVLTNKLPKVKKEFGEDSEEYQDIKSKLKFIEQQIKQSSVLKGKNLSAGIDPGVQAVVSTDHGALF
LPNLTRERISIHIEELQSRLDNAELINDKKWKSLGNKTSRIKTKNESKLQQKISRLHERGANSSNAFNHKLSTRLSRTY
EHISWEDTQVTNLLKQVDPKALPEGVGYAHNGASAKRGLNWIMRQRCLGDLKAKTKQKTENRGGNFHEPPANYSSQICH
CCGQKGERRSQYEFVCKNPDCKLFDVAQQADTNAARNHKQNGGFELGEVKYHNVRLVYQKPKRFKKKRLTK
(SEQ ID NO: 625)
>3300001043|JGI12316014372_10004500_6|M
[aquatic-marine]
MKTLEFKLDLTQEQQALINQWLADLKWVWNRGLRLLEEDQQRRWRDKHGHLLPDSLKLQWRNGQPTGSGVRKTRDGYKY
CAIRQDRDVEDVRKFFSSDSYHNKKNTPQWLHDVPAKFRVGVSAQLKQAWKSYQDPKHPAKRPRYKGKRDCLRSLVNRN
AGGKNKELNPEQIGDSWNAYVRFPKLGKLKVKGFYRRFFPNDGWGSARILVEPSGYYLQVAVEQPDEVVKESDKAVGID
PGVANTLTLDNGRHVKPNAKLDRHQKRLLRLQRRASRQKRGSNSQQRTHQAIARQHEKIRRSRNAFNHKLSTKLTREYG
AIAFEDTQLQNMTRRSKPKPREDGKGWEQNGAKRKTGLNRSLANIAIGDLKQKTKDKATALGREFITPDAKYSSQECHV
CGERGERLSQSVFICLNPDCKEFRCEQHADTNAARNLLLRALPDLEREYRPWGWELKREESVSPTGGTTSNGETPSKLG
MPPEAVTVLC
(SEQ ID NO: 626)
>3300001043|JGI12316J15309_10000277_14|M
[aquatic-marine]
MKTLEFKLDLTQEQQALINQWLADLKWVWNRGLRLLEEDQQRRWRDKHGHLLPDSLKLQWRNGQPTGSGVRKTRDGYKY
CAIRQDRDVEDVRKFFSSDSYHNKKNTPQWLHDVPAKFRVGVSAQLKQAWKSYQDPKHPAKRPRYKGKRDCLRSLVNRN
AGGKNKELNPEQIGDSWNAYVRFPKLGKLKVKGFYRRFFPNDGWGSARILVEPSGYYLQVAVEQPDEVVKESDKAVGID
PGVANTLTLDNGRHVKPNAKLDRHQKRLLRLQRRASRQKRGSNSQQRTHQAIARQHEKIRRSRNAFNHKLSTKLTREYG
AIAFEDTQLQNMTRRSKPKPREDGKGWEQNGAKRKTGLNRSLANIAIGDLKQKTKDKATALGREFITPDAKYSSQECHV
CGERGERLSQSVFICLNPDCKEFRCEQHADTNAARNLLLRALPDLEREYRPWGWELKREESVSPTGGTTSNGETPSKLG
MPPEAVTVLC
(SEQ ID NO: 627)
>3300001341|1010336|scaffold_14_1570|P
[aquatic-marine]
MKTLEFKLDLTQEQQALINQWLADLKWVWNRGLRLLEEDQQRRWRDKHGHLLPDSLKLQWRNGQPTGSGVRKTRDGYKY
CAIRQDRDVEDVRKFFSSDSYHNKKNTPQWLHDVPAKFRVGVSAQLKQAWKSYQDPKHPAKRPRYKGKRDCLRSLVNRN
AGGKNKELNPEQIGDSWNAYVRFPKLGKLKVKGFYRRFFPNDGWGSARILVEPSGYYLQVAVEQPDEVVKESDKAVGID
PGVANTLTLDNGRHVKPNAKLDRHQKRLLRLQRRASRQKRGSNSQQRTHQAIARQHEKIRRSRNAFNHKLSTKLTREYG
AIAFEDTQLQNMTRRSKPKPREDGKGWEQNGAKRKTGLNRSLANIAIGDLKQKTKDKATALGREFITPDAKYSSQECHV
CGERGERLSQSVFICLNPDCKEFRCEQHADTNAARNLLLRALPDLEREYRPWGWELKREESVSPTGGTTSNGETPSKLG
MPPEAVTVLC
(SEQ ID NO: 628)
>3300027607|Ga0207422_1002635_7|P
[aquatic-marine]
MKTLEFKLDLTQEQQALINQWLADLKWVWNRGLRLLEEDQQRRWRDKHGHLLPDSLKLQWRNGQPTGSGVRKTRDGYKY
CAIRQDRDVEDVRKFFSSDSYHNKKNTPQWLHDVPAKFRVGVSAQLKQAWKSYQDPKHPAKRPRYKGKRDCLRSLVNRN
AGGKNKELNPEQIGDSWNAYVRFPKLGKLKVKGFYRRFFPNDGWGSARILVEPSGYYLQVAVEQPDEVVKESDKAVGID
PGVANTLTLDNGRHVKPNAKLDRHQKRLLRLQRRASRQKRGSNSQQRTHQAIARQHEKIRRSRNAFNHKLSTKLTREYG
AIAFEDTQLQNMTRRSKPKPREDGKGWEQNGAKRKTGLNRSLANIAIGDLKQKTKDKATALGREFITPDAKYSSQECHV
CGERGERLSQSVFICLNPDCKEFRCEQHADTNAARNLLLRALPDLEREYRPWGWELKREESVSPTGGTTSNGETPSKLG
MPPEAVTVLC
(SEQ ID NO: 629)
>3300007960|Ga0099850_1030516_|P
[aquatic-marine-aqueous]
MNTITKVRKIEFKVTLDAQQRQVFEHHSRDLRFIWNTGLACIQWREWWEKWQQVLESPDRVGYEPETVPINWACNIVKS
PKKKKKDEESEKKVYALACDRVQYRLEKTTNLELGTGEWTEPRKDRNGKIDEDHCWVAYPCKPEPVREHWTEEPRLLLL
MKNPYMALRTMFAKKNWDPETVEGKIIHGLNTAWVAGECKALGDAWSSYKSGVRKRPRFKKRHDRSNLINPSSKQTKFS
GNSLTLPGLGRVTVKGLQQRWGEKPACPIALIKEGDEFYLQLTGELEIPVVRSNKVTIGIDPGSVRLATTDNGAVIEAH
KFGKQMTRRQKKLQASINRKLRLNSDRVYDESDSSRFYLKPRKNWKRKNLQKVRTKLQKLSAKTARQRRAYNHFHSTRI
VQSADVIIMEGLELKNVTRAVKKGKSGEHNKRKAKAGLNRSLLDNAIGQFYGMIEQKAKDAGKVTARVNPFRTSMTCNK
CGFSDAKNRKTQAGFHCQKCGHSDNADRNAALNIKRMGVLGVTKLTFDNDWNITVPGWIEPGDLVALHSKGLKPVGTAK
KRSKRAGKEHPSQLALEIGSQ
(SEQ ID NO: 630)
>3300028735|Ga0272446_1017865_2|M
[aquatic-thermal springs-microbial mat]
MSQNNLTPKNIQHGCRLTKMQQQICEQWLDELRRLWNEALSYLLEVEENTVYNFADKCRYAVCKLSTEYRHYRWDDADG
EPPINHYKVEESVDESRKKRTAGTYIFVPYTVCVAPKKPYAPICPIPQGHNHLDWTGSSGTFPEGFLRLDPSKLNRATG
YAKLALLQKFTHKNCPHLKHIPANFIRGVLFSLATAWEKRFQRRDKYGRPGYPKFKSRKFPLTSLQYVDAVNLKREGDS
LRFPVLGCVTARGISERFPVDIEIKVATLQKQPDGWYMIWNGWFPAIVAKPKNRSIAISFRQNNNLYVADNGYVMMPYQ
PPEQKLRRLERLRQILCGKQFLSKNWHRVKAKIAKYEHRLAMARRNHNHKISTFLVRKYDQLVVDNRKLGLIRKPEPIP
VGVTPPRWAPNGATAVSEINQRKAHNAKGQFRNMVIEKSKTYQRAVRLAPVEPPSNTPS
(SEQ ID NO: 631)
>SRR6869054_1535443_5|P
[biofilm metagenome]
MKTLEFKLDLTQEQQALINQWLADLKWVWNRGLRLLEEDQQRRWRVKHGHPLPDSLRLKWRNGKLVGCGIRKSREGYLC
CPVRQDRDIEDARKLFASDSYHNKRNAPQWLHDVPARFRVGVRAQLKQAWKSYQDPKHPARRPRYKGKRDRLRSLTNLN
AGGKKKELSPEQIGDSWNAYVRFPKLGRLKVKGFYKRFLPNDGWGSARILVEPSGYYLQVAVEQPDEPVKESDKAVGID
PGVANTLTLDNGRHVKPNAKLDRGQKRLLRLQRKASRQQRGSNSQQRTYQAIARQHEKIRRSRNAFNHKLSTKLTREYG
AIAFEDTQLQNMTRRSKPKPREDGKGWEQNGAKRKTGLNRSLANIAIGDLRQKTKDKAAELGREFVTPDAKYSSQECHA
CGEKGERLSQSVFICLNPDCKEFNCEQHADTNAARNLLLRALPDLEREYRPWGWELKREESVSPTGGTTIFGETPSKLG
MPPEAAMVSV
(SEQ ID NO: 632)
>SRR6869054_457352_2|P
[biofilm metagenome]
MILCQEFAVELSPPQEKNAIAWLEASKSLYNSALYELLRWDENTGFYCKETKSYLPCCSVPWEYRWHKESETLIPFSRI
ASEKSRWYIKNLPRHRVPTPAEEKDSWGWHCKDGGSGYSCPIPKDYCEPLLTRWERQARGGLGLVAKGDYWEKGHSVLD
IPYKFRSGVLTMLETPWQEYLKSRFGQTTVKRGCPRFKRKRDRINTLIHPNPKGVIVPNRNQLKGVPKLGSLKVKGLVR
RWRNPDGFVPPISIFKLIRRQGQWFVQLTGDLERSYPVKNTNRALGIDPGLKLEFGTSDGWSLPAARRYRKGQKQREKL
QKQIAHKRKHNLVLWLNQAERQLQDVAGTIIPLKAESWAALQQCRTEKEFINLIGAARYNRLKYSVPTSNKILKLEERA
AKHEARIARTRKAADDKLTSRIVREYGFIAIEHGLQGQKLRGKAKAKKREDGKGYAQTGAKRKSGVSKSLSDASIGRKT
AMLERKAKRSNRTFAKIESAYTSQSCPVCGHLHKAPMDGDRVYHCECCGWHCDRDQSAGINIELMAYAANSTAKLSSAA
ELARVYGCFWMQDNPKETKPLWAKKLSKKKLAEWMKNIRASYPDKVLQNFELEYRKMKAQK
(SEQ ID NO: 633)
>SRR6869055_1317318_5|P
[biofilm metagenome]
MKTLEFKLDLTQEQQALINQWLADLKWVWNRGLRLLEEDQQRRWRVKHGHPLPDSLRLKWRNGKLVGCGIRKSREGYLC
CPVRQDRDIEDARKLFASDSYHNKRNAPQWLHDVPARFRVGVRAQLKQAWKSYQDPKHPARRPRYKGKRDRLRSLTNLN
AGGKKKELSPEQIGDSWNAYVRFPKLGRLKVKGFYKRFLPNDGWGSARILVEPSGYYLQVAVEQPDEPVKESDKAVGID
PGVANTLTLDNGRHVKPNAKLDRGQKRLLRLQRKASRQQRGSNSQQRTYQAIARQHEKIRRSRNAFNHKLSTKLTREYG
AIAFEDTQLQNMTRRSKPKPREDGKGWEQNGAKRKTGLNRSLANIAIGDLRQKTKDKAAELGREFVTPDAKYSSQECHA
CGEKGERLSQSVFICLNPDCKEFNCEQHADTNAARNLLLRALPDLEREYRPWGWELKREESVSPTGGTTIFGETPSKLG
MPPEAAMVSV
(SEQ ID NO: 634)
>SRR3137750_862387_23|M
[crustacean metagenome]
MPKAKPTDKEKDKRPIKTLEFKLYPNQSQVLTLTDWLDRGRKVWNNGLAVLLEKDQQDWRKKAGFDPMEGSEAWQWFPN
QVGSKTVFGACCAITFYNRRQNEHYPACPIRHPQDVDGAPRSLIYRATQLDNFCTRFKGGIGDDLLESWKAYKDKNRPT
AKLPKFKGKQFPLKSLSNGECTAKIVGKTAIKFPLLGNIRTKGLERVPQDSKICTARICRKASGWYLQLAIRWDWPVPN
VKMPDVAVAIDPGVRFATSTDYGRQVDAPQFMLKQTKKLRREQRKLSRRKLKGKNWQKQAAKIARLHEKVSRQRNAFWH
KESTYFVTSFGGVAIEDNNFNNMGRKAKAKPKEDGKGYERNNQAQKRGLNRSLKDVACGRLKVMVETKAKTINNEVHLV
RSHHNSQTCSECGHVAKDNRKSQSNFSCVACGHSLNADINAAINIYNRAAWSNRYNLHPVRLQVSNLERYPSFVGGIGK
SHKPASASRKSRNGIEGQQEGGSLPPLEITKESRSPGLTRNKPASVSPSIGKPKRGQKPSTLCQIPETHTQLTLWDLTG
(SEQ ID NO: 635)
>SRR3138838_558748_1|P
[crustacean metagenome]
MENMTRVIKFRLTLTQNQQSLIDEKMRLLKYLWNLAVGLQEEVNQRRLRDKFEFKEFSPEFWETSYLSLYSSFPHIEKL
PKDKIKLLAGNPCCKIAYLKNKKEYVSLKLEERKKRPYLGNPDAKKHTTKPPDKEDLKKYFYSDLADKIFEIGSEFRGC
FYRYVISKAFETAKKGIRGFPKYKGKLDSVQSLIVGHPTLIKVRDGGIFLNKQIGLLVIDDINRLPKESDVRMASIVRK
ASGYYLHLTINLPVPMHFPSDRQIGIDVGCVNLYTDSEGRHIETKAYYRNKERKIAHLQRKMARQEKGSANYLKSKEKL
ALLHEQIKDRRAGYLHQVSHKLTQKYSHIAMEDLKLKNMTKRAGVKENEDGTYAHNGASAKSGLNKSILDQGIGELKRQ
LIYKSQEKGGRVVLVNPKYTSQTCFNCGHKHKDNRKSQSEFICVSCGYSANADENAAKNILTLSLISSTIGVLRSSLLS
YPSLLGKAVSLLAYVCGSPKGTCEAERERSDPPINPPVKGGNCLDNFSTLTINPRHQSEKVSQPIDSVQLTIW
(SEQ ID NO: 636)
>SRR3139299_296800_1|M
[crustacean metagenome]
MSNFSENVRTKEFKLILSSEQERTLEDWMLVCKWVWNRSLGLIEEFNEWNPYDKLSKSNVPATPLQRYDRKLKQWVRIE
IPDWKMGIERVEKKRGLIHPVAIDENSPIIDSLDSKKSVLYGFLKVFGHQHHKDRIVTYLVRGEERKVNFTDCPAKFIQ
GVAHELSKAWEGFLAGRHSRPRFKTAKDKVMTLIHYNAKDLGVKDSKVNIPKLGYIEVIGFDKRWYGCDFNPMKICKKA
SGWYIQLTASVTVKQAKKTGLCCGIDPGHQFVMALDNGHTIEAAQPLKRSLKRLRKMQQQLSRKYRMNEGKTKNWEKLN
NKIAKLHEKIARHRRSFNHWHSTNLINWFDVIFVEDYKPANVYRKAKAKAKLDTEGNPVVAENGTVIYDKNSQKRKRGS
NRTGSDVAIGQAIDLLETKAKEHGKLVIRVDNWGTTLCCAKCGHQEKKKLSQRTHKCSNCGYTVARDVNSGQNIKLKGL
AQMAINQGVELSDNFWYKFLINKNSATPSCSKKATKKKPKKSDTITIPGDLFKNGVHDFLVAETAKTHAIINQMSLGDF
DRNA
(SEQ ID NO: 637)
>SRR3139690_493772_1|M
[crustacean metagenome]
MLPKRHAYIGATCELIRDGGWWAKDESATPIAYRGKKLSADKLAKTKESDIWWSDGVAHERLYKFPMSKWMPPKPGKIP
QDVANKPFQYMRVPSTLGGGLEIKKFEQLNNAKHLKEFGIHLPPGVSHFLCGLFAQFSESYKAWIDPKRPNAHKPRYRQ
PDDLMRSLYSNQMRYSTTYGCNAAPIEFLSRDGVGVIAMNSIFGDVEMYRGEIDRMPDGLIPRSFNFTQDASGYYISVV
FCTMAEVNNTFAKAQYRRFAGKADEDRKAELKAAVDDAAAALKAETYNPGNGKTLGIDPGIKRQITAHDGEKTFHVKLS
KRRHDKARRLVNRIDRLKSKLSRIKTANNIRLNAGKDLRRAAGVITIDGVTTTLKNEAKLQHRIRRLQLLQANMRRAYQ
HRVAVRLFRGGYSTIRFEATQVGNMSKRSKVKIKANGKYAKNNAAAKSGLNKSLANTAMAAQSTKIEARFKAAGRSFIA
VPAHNTSQICHCCQIKGDRASQELFLCLNNLCVMAGIRQNADDNAAKNMYRETLATEPKRSTGSGLELGEEGDIDD
(SEQ ID NO: 638)
>SRR3139690_185157_1|M
[crustacean metagenome]
MDTSIVDQQQTETELSVIEFKLSPHNEAVKAWEYQLNELKTVWNSALALRIEADDRYWIKRLNLDAVCPSTALKRKGNK
KQGKGQSPTQWRTIASGVVRPEKTKPIRARGAKTGIIAGPYCRVRKLQGVEPLDKQIIDADGNKITSALSRTQAISNYP
WLDSSLVDSRYIAGVYRTLDQAWKAYKKGVRNRPKFKGKKDKLKSLANYSGSVRFERIGGGPNGWVNFPKLKPVKCKGL
YKRYDDRMKTGTVKLCKKGGNWYLQLTRKDVPFNRLSDSDKAIGIDLGVKQNATTSEGKVYHCKQSKRIQQRIERLQRK
AARQYRMNADEKHPTGRSTLGHQRTIGEISRLQDKQKRSRHAWQHKTSTKILKSNGVIVFEDLKLQNMTKRPKPKSDEK
GGYAKNGAKAKAGLNRVILKSGLGGLRSKVESKGDRNNRTVIRVNPAYTSATCSNCGHRHTKEEKASYRPTQSQFICQK
CGHTDNADINAAENILVSGLTMLDTV
(SEQ ID NO: 639)
>SRR3139690_493772_1|P
[crustacean metagenome]
MKAIRFKVCPTDEQVRSFNDLRSDLTQVWNLIHRVAMHNRSVEWFRWAEKTSSKDGSWDLHDCSLTPIMLPKRHAYIGA
TCELIRDGGWWAKDESATPIAYRGKKLSADKLAKTKESDIWWSDGVAHERLYKFPMSKWMPPKPGKIPQDVANKPFQYM
RVPSTLGGGLEIKKFEQLNNAKHLKEFGIHLPPGVSHFLCGLFAQFSESYKAWIDPKRPNAHKPRYRQPDDLMRSLYSN
QMRYSTTYGCNAAPIEFLSRDGVGVIAMNSIFGDVEMYRGEIDRMPDGLIPRSFNFTQDASGYYISVVFCTMAEVNNTF
AKAQYRRFAGKADEDRKAELKAAVDDAAAALKAETYNPGNGKTLGIDPGIKRQITAHDGEKTFHVKLSKRRHDKARRLV
NRIDRLKSKLSRIKTANNIRLNAGKDLRRAAGVITIDGVTTTLKNEAKLQHRIRRLQLLQANMRRAYQHRVAVRLFRGG
YSTIRFEATQVGNMSKRSKVKIKANGKYAKNNAAAKSGLNKSLANTAMAAQSTKIEARFKAAGRSFIAVPAHNTSQICH
CCQIKGDRASQELFLCLNNLCVMAGIRQNADDNAAKNMYRETLATEPKRSTGSGLELGEEGDIDD
(SEQ ID NO: 640)
>WP_015957291.1
[Cyanothece sp. PCC 8801]
MFAIKSMSELVQHHITIQLKAYLSTTQTALFENWTDSLRPLYNLALGLLYEEQQRRWRTNQKFLKNYLDKSSLQTYLNE
IENKPDIYPVEWHITKALPECDWLTKEENEVRKKDNTKSLACRTINRDGNFFTPIRPYWHLEEPQKLAKFKCFTNQWLI
SCNLLTNYHLQKLLNVNMKVRQSFISMNLMEAWKRYQKGDFRKLKFKSKRNPVISLCNKQTNRIKFDPEANNCQLLGKE
FGLIEFRGLHNRHQGQIQPRNGSLTKKADGYYLNLVFQVEHKPIPDSDLQVGIDPGLVTLLTLSDGKCISNQRFLKENE
RHLTVLQKKLSRQTPGSKNWEKTKKALAKIHKQTADHRKYYNHKVSTHLVNKYGAIAIEDTKLTNMNKRPKAEKREDGK
GYEHNGAKAKAGLNQSFHDAGLGQLRAFLESKANSYENRHIERVRANYTSQKCSRCGHTDSENRLTQASFHCLKCGLEM
PADLNAAINIEQTAFGLDKS
(SEQ ID NO: 641)
>CP001287_4134|P
[Cyanothece sp. PCC 8801]
MKEKHGLVDQAMFAIKSMSELVQHHITIQLKAYLSTTQTALFENWTDSLRPLYNLALGLLYEEQQRRWRTNQKFLKNYL
DKSSLQTYLNEIENKPDIYPVEWHITKALPECDWLTKEENEVRKKDNTKSLACRTINRDGNFFTPIRPYWHLEEPQKLA
KFKCFTNQWLISCNLLTNYHLQKLLNVNMKVRQSFISMNLMEAWKRYQKGDFRKLKFKSKRNPVISLCNKQTNRIKFDP
EANNCQLLGKEFGLIEFRGLHNRHQGQIQPRNGSLTKKADGYYLNLVFQVEHKPIPDSDLQVGIDPGLVTLLTLSDGKC
ISNQRFLKENERHLTVLQKKLSRQTPGSKNWEKTKKALAKIHKQTADHRKYYNHKVSTHLVNKYGAIAIEDTKLTNMNK
RPKAEKREDGKGYEHNGAKAKAGLNQSFHDAGLGQLRAFLESKANSYENRHIERVRANYTSQKCSRCGHTDSENRLTQA
SFHCLKCGLEMPADLNAAINIEQTAFGLDKS
(SEQ ID NO: 642)
>WP_015785111.1
[Cyanothece sp. PCC 8802]
MFAIKSMSELVQHHITIQLKAYLSTTQTALFENWTDSLRPLYNLALGLLYEEQQRRWRTNQKFLKNYLDKSSLQTYLNE
IENKPDIYPVEWHITKALPECDWLTKEENEVRKKDNTKSLACRTINRDGNFFTPIRPYWHLEEPAKLAKFKCFTNQWLI
SCNLLTNYHLQKLLNVTMKVRQSFISMNLMEAWKRYQKGDFRKLKFKSKRNPVISLCNKQTAIIKFDPEANNCQLLGKE
FGLIEFRGLHNRHQGQIQPRNGSLTKKADGYYLNLVFQVEHKPIPDSDLQVGIDPGLVTLLTLSDGKCISNPRFLKENE
RHLTVLQKKLSRQTPGSKNWEKTKKALAKIHKQTADHRKYYNHKVSTHLVNKYGAIAIEDTKLTNMNKRPKAEKREDGK
GYEHNGAKAKAGLNQSLHDAGLGQLRAFLESKANSYENRHIERVKANYTSQKCSRCGHTDKENRLTQASFYCLKCGLEM
PADLNAAINIEQAAFGLDKS
(SEQ ID NO: 643)
>NC_013161_4122|P
[Cyanothece sp. PCC 8802]
MKEKHGLVDQAMFAIKSMSELVQHHITIQLKAYLSTTQTALFENWTDSLRPLYNLALGLLYEEQQRRWRTNQKFLKNYL
DKSSLQTYLNEIENKPDIYPVEWHITKALPECDWLTKEENEVRKKDNTKSLACRTINRDGNFFTPIRPYWHLEEPAKLA
KFKCFTNQWLISCNLLTNYHLQKLLNVTMKVRQSFISMNLMEAWKRYQKGDFRKLKFKSKRNPVISLCNKQTAIIKFDP
EANNCQLLGKEFGLIEFRGLHNRHQGQIQPRNGSLTKKADGYYLNLVFQVEHKPIPDSDLQVGIDPGLVTLLTLSDGKC
ISNPRFLKENERHLTVLQKKLSRQTPGSKNWEKTKKALAKIHKQTADHRKYYNHKVSTHLVNKYGAIAIEDTKLTNMNK
RPKAEKREDGKGYEHNGAKAKAGLNQSLHDAGLGQLRAFLESKANSYENRHIERVKANYTSQKCSRCGHTDKENRLTQA
SFYCLKCGLEMPADLNAAINIEQAAFGLDKS
(SEQ ID NO: 644)
>OVJI010000022_17|P
[freshwater bacterium LH6-2 Bacteria.]
MKTICVKVRLTPEQEQEFKSAIDELEWIWNFSLKVHLYNHCIKWYDWAANTKQKIDKAIAEMDKLPPKQKEFVHNYFLG
DPQLRPTKLDQSQRKLVDKEVYKILERWSPFDLEGIEKVPLRISERSAYFNASCVIAENGPYWFSEVIEQTDDQGEVIG
KTKKWKLAQGRKPYTPLPKIPHKYILAPSGQYKDREIIDWTDLVVKAVLTSVRESEGLPPLNIHSNYINGLVSQNGSLQ
KAWVAFLDTSRQQSKRPKFKNEENRLNTLLNLYPPTILDEENSIEVRQLGKLEVVDRNWRKRLGQNISLRSHCITKKRS
GYYVHVTVEHALQSEKTILVKQLTKIKKEKGVESQEYVELSEKITEMENTIRDSKVRLNKKQLSVGVDPGIQAVIATDQ
GDLYTPNLSRERITMHLEKLQHKLKHMQEINNAAWRQENTHKIEAGEQIDIKEKRPKTKNELKLEHKIARLHERGASSA
RHFNHKLSSRIARTYTHVCWEDTKLENLLKQCKAKPDLSGIGYLPNGAAAKRGLNWLLKQRCLGDLKERTKQKVLEYGG
VWHNSAPKYSSQRCHCCGYEEPLQRDGSNFTCKNQDCELFGVSQNADVNAARNHKKSVFELGQVKYHNLSLQYNISARR
RHRNRNKPKLTVVKLPE
(SEQ ID NO: 645)
>CAAAFE010002307_2|M
[freshwater metagenome]
MVTNRRIEFKVSLDQQQQQIFDHHIAKLQAVWNIGLACIEWREWWEKWQQVLESPDHPDYAPDAVPMGWGHNSIKGKKK
PVYGLCCDRVKFRLEKNDPILDVGEWVEGTKKGSGDSIAGYWVAYPAKPDPVKEHWVEDPRLPKHGQDPYMSLVSMFGK
KYWPSDHFIQGLSSAWIKGQCKNLATAWKAYKKGIRKRPQFKKRHEAGNSLLCVTRIPLKQNGMTLPGMGFVRIPKLAQ
RWDSIKDPCPISLLREGSRYYVQLTATLEQRPVRSNGVTVGIDPGSVHVATDDRGRMIDSPRYAKKQERRKRRLQRSMA
RKFRLNTTTVYDEDGRVLRHEPRKGWKRRNHAKVKQQLQSLEAHTKRARRAFNHFHSTRIVQSADVIVLENLQLQNIGA
KVKTGKTGISNGRKRKTGLNNKLRDNAIGQLFAMVEQKAAAAGKLTTRVNPFRTSMTCNRCGFSDKANRVSQAVFHCQS
CGHRANADQNAAANIRKMGLLGVTKLTFDNDWRITVPNWHD
(SEQ ID NO: 646)
>OGWG01000020_5|P
[freshwater metagenome]
MTIMKTICIKVKLSPEQEREFSNATQELEWIWNFSLKTYIHNHCVKWYEWATGIKVKIDKALAEIEKLPPEQKEFVYNY
FLNPSLRPSKMTQQQNKLVDKEVYKILTMWSPFDFDGITKVPLRLSERSAYIGASCAIAEGGPYWISEIIEEKDESGEV
IKHRKWKLVNGKPYMPINKKEHSYPLVPSGQYKDREIIDWTDLVVKAVLTSVRESEGLSPLTLHSNYINGLVSQNGSFQ
KSWSAFLDVKRMQSKRPKFKNEEYKLNTLLNLYPPTISEDEDWIEVRQLGKLEVIDRNWRKRMGGDSPRSHCITKKKSG
YYVNITTEHPLHSEKTVLLKQLAKIKKEQGKDSQEYLDLEATIASMTEKIRGSKVKPKNKELAVGIDPGINAIIATDQG
DLYMPNLSRERITFHIEKLQAKLKHMQDVNDKVWRQENQRQLEDGEKLEIKTKRPKTNNELKLEHKIARLHERGASSSR
HFNHKLSSRIARTYKHVCWEDTKIENLMKSPKAKPDLSGIGYLPNGAAAKRSLNWLFKQRCLGDLKERTKQKVLEYGGV
WHDSAPKYSSQKCHCCGYTEPLQRDGSNFTCKNEACKMYGISQSADVNAARNHKKSVFELGEVKYHNLSLEYNVSARWK
HRNRNKVKPVVSVTKLPTTSTPVPNKPK
(SEQ ID NO: 647)
>OGWG01000020_4|M
[freshwater metagenome]
MKTICIKVKLSPEQEREFSNATQELEWIWNFSLKTYIHNHCVKWYEWATGIKVKIDKALAEIEKLPPEQKEFVYNYFLN
PSLRPSKMTQQQNKLVDKEVYKILTMWSPFDFDGITKVPLRLSERSAYIGASCAIAEGGPYWISEIIEEKDESGEVIKH
RKWKLVNGKPYMPINKKEHSYPLVPSGQYKDREIIDWTDLVVKAVLTSVRESEGLSPLTLHSNYINGLVSQNGSFQKSW
SAFLDVKRMQSKRPKFKNEEYKLNTLLNLYPPTISEDEDWIEVRQLGKLEVIDRNWRKRMGGDSPRSHCITKKKSGYYV
NITTEHPLHSEKTVLLKQLAKIKKEQGKDSQEYLDLEATIASMTEKIRGSKVKPKNKELAVGIDPGINAIIATDQGDLY
MPNLSRERITFHIEKLQAKLKHMQDVNDKVWRQENQRQLEDGEKLEIKTKRPKTNNELKLEHKIARLHERGASSSRHFN
HKLSSRIARTYKHVCWEDTKIENLMKSPKAKPDLSGIGYLPNGAAAKRSLNWLFKQRCLGDLKERTKQKVLEYGGVWHD
SAPKYSSQKCHCCGYTEPLQRDGSNFTCKNEACKMYGISQSADVNAARNHKKSVFELGEVKYHNLSLEYNVSARWKHRN
RNKVKPVVSVTKLPTTSTPVPNKPK
(SEQ ID NO: 648)
>OGWL01001214_2|P
[freshwater metagenome]
MNTITKVRKVEFKVALDVQQQQVFEHHSRDLRFIWNTGLACIQWREWWEKWQQVLESPDRVGYAPEPVQMDWGYNLVGK
KKVYGLVCDRVRFRLEKSEPVLAAGEWVAGEKQGGGYWVAYPCKPEPVREHWTEEPRLPLLMKNPYMALRTMFAKKNWD
PETIGGKIIHGLNTAWVAGECKALGDAWSSYKSGVRKRPRFKKRHDRSNLINPSSKQTKFSGNSLTLPGMGRVAVKGLQ
QRWGDKPACPIALIREGDEFYLQLTGEVQVPAVRSNKITIGIDPGSVRMATTDNGAVIDAPKFGKRIKRRQEKLQASIN
RKLRLNSDRVYDESDSSKFYLKPRRNWKRKNLQKVRTKLQRLSAKAARQRRAYNHFHSTRIVQSADVIIMEGLELKNVT
RAVKKGKSGEHNKRKAKAGLNRSLLDNAIGQFYGMIEQKAKDAGKATTRVNPFRTSMTCNKCGFSDAKNRKTQAGFHCQ
KCGHSDNADRNAALNIKRMGVLGVTKLTFDNDWNITVPGWVEQKTVEPLQVRDFKPVGTVKKRSKKTEQKRSSQMALEI
GSP
(SEQ ID NO: 649)
>OGW001000325_8|P
[freshwater metagenome]
MDNYIETTRTKEFKLILTPEQERTLENWMLVCKWVWNRSLGLIEEFNDWNPYDKLSKSYAPAMPLQRYDRKLKQWVRVE
IPEWKLNTKTKETSKGLVHPVSIDENSPQIDSLNSKKSVLYGFLKIFGHQYHKDRIITYMVRGEERKVKFTDCPAKFIQ
GVSHELSKAWDGFLAGRHSRPRFKTAKDKVMTLLHYNAKDIGIQGSKINIPKLGYVEVIGFDKRWYGVDFNPMKICKKS
SGWYIQFTAVVPVKKFKKTGLACGIDPGHQFVMALDNGHIIKAAQPLKQNLRRLRRMQKQLSRKYRMNNGKTKNWEKLN
QRIAKLHEKIARHRRSFNHWHSTNLVKWFDIIFVEDYKPANVYRKAKAKAKTDEQGNPVTAENGAVIYEKNNQKRKRGN
NRTGRDIAIGQAIELLETKAKEHGKLVIRVPAYGTTLCCAKCGHEERKSLSQRVHHCSSCGYIVARDVNSGQNVKLKGL
AQLALDHGVELSDNFWYQILKNATIDSCSGKKGKKTTKKKDAVSIPGDLFKNGIAAFLITETAKTRAIIERNHLENLNK
TAKSLMG
(SEQ ID NO: 650)
>OGWP01000327_5|M
[freshwater metagenome]
MKTICIKVKLSPEQEREFSNATQELEWIWNFSLKTYIHNHCVKWYEWATGIKVKIDKALAEIEKLPPEQKEFVYNYFLN
PSLRPSKMTQQQNKLVDKEVYKILTMWSPFDFDGITKVPLRLSERSAYIGASCAIAEGGPYWISEIIEEKDESGEVIKH
WKLVNGKPYMPIRKNKKEHSYPLVPSGQYKDREIIDWTDLVVKAVLTSVRESEGLSPLTLHSNYINGLVSQNGSFQKSW
SAFLDVKRMQSKRPKFKNEEYKLNTLLNLYPPTISEDEDWIEVRQLGKLEVIDRNWRKRMGGDSPRSHCITKKKSGYYV
NITTEHPLHSEKTVLLKQLAKIKKEQGKDSQEYLDLEATIASMTEKIRGSKVKPKNKELAVGIDPGINAIIATDQGDLY
MPNLSRERITFHIEKLQAKLKHMQDVNDKVWRQENQRQLEDGEKLEIKTKRPKTNNELKLEHKIARLHERGASSSRHFN
HKLSSRIARTYKHVCWEDTKIENLMKSPKAKPDLSGIGYLPNGAAAKRSLNWLFKQRCLGDLKERTKQKVLEYGGVWHD
SAPKYSSQKCHCCGYTEPLQRDGSNFTCKNEACKMYGISQSADVNAARNHKKSVFELGEVKYHNLSLEYNVSARWKHRN
RNKVKPVVSVTKLPTTSTPVPNKPK
(SEQ ID NO: 651)
>OGWP01001429_1|P
[freshwater metagenome]
MHTTKTRKIEFKVRLDLQQQQVFEHHSQALRFIWNTGLACIHWREWWEKWQQVLESPDRPGYCPDPVDMGWGHNLVGEK
KVYGLCCDRVRFRLEKEEPVLGVGEWIEGEKLGSGKRVAGYWVAYPAKPDIVREHWEQEPRLVLLMKNPYKALKTLFAK
KNWDPDSVEGKIIHSLNTSWVAGECKALGDAWSSYKSGVRKRPRFKKRHKRSNLINPSSKQTKFFKNSLTLPGMGRVTV
KGLDKRWGNKPACPIALIKEGSDFYLQLTGAVEVRTVRSNRSNGVAIGIDPGSVHVATDDHGKTIEPPRYGKKAMRRIQ
KLQASINRKLRINSEKVYDPENPEKFYLKPDKNWKRKNLKKVRAKIQRLSAKVVRSRRAFNHFHSTRLVQSADTIVLED
FKLKNVTRAVKEGESGVQNGRKAKAGLNKNLLDNAIGQLYGMIEQKAKDAGKMTARVNPFRTSQTCNRCGFSDGKNRPS
QAVFRCQKCNHCDNADRNAALNIKRMGMLGVTKLTFDNDWNITVPGWVAAAPAEPSKPHKPVKPADSSLSKDLKPKNDC
PSARSKPKTIEGSQMKLAL
(SEQ ID NO: 652)
>OGWP01001429_1|M
[freshwater metagenome]
MKPFLSIPSMHTTKTRKIEFKVRLDLQQQQVFEHHSQALRFIWNTGLACIHWREWWEKWQQVLESPDRPGYCPDPVDMG
WGHNLVGEKKVYGLCCDRVRFRLEKEEPVLGVGEWIEGEKLGSGKRVAGYWVAYPAKPDIVREHWEQEPRLVLLMKNPY
KALKTLFAKKNWDPDSVEGKIIHSLNTSWVAGECKALGDAWSSYKSGVRKRPRFKKRHKRSNLINPSSKQTKFFKNSLT
LPGMGRVTVKGLDKRWGNKPACPIALIKEGSDFYLQLTGAVEVRTVRSNRSNGVAIGIDPGSVHVATDDHGKTIEPPRY
GKKAMRRIQKLQASINRKLRINSEKVYDPENPEKFYLKPDKNWKRKNLKKVRAKIQRLSAKVVRSRRAFNHFHSTRLVQ
SADTIVLEDFKLKNVTRAVKEGESGVQNGRKAKAGLNKNLLDNAIGQLYGMIEQKAKDAGKMTARVNPFRTSQTCNRCG
FSDGKNRPSQAVFRCQKCNHCDNADRNAALNIKRMGMLGVTKLTFDNDWNITVPGWVAAAPAEPSKPHKPVKPADSSLS
KDLKPKNDCPSARSKPKTIEGSQMKLAL
(SEQ ID NO: 653)
>SRR3989431_2283179_3|M
[freshwater metagenome]
MSNFSDNVRTKEFKLILSSEQERTLEDWMLVCKWVWNRSLGLIEEFNEWNPYDRISKSNVPATPLQRYDRKLKQWVRIE
IPDWKMGIERVEKKRGFIHPVAIDENSPVIDSLDSKKSVLYGFLKMFGHQHHKDRIVTYLIRGEERKVNFTDCPAKFIQ
GVAHELSKAWEGFLAGRHSRPRFKTAKDKVMTLIHYNAKDLGVKDGKINIPKLGYIEVIGLDKRWYGCDFNPMKICKKA
SGWYIQLTASVPIKQAKKTGLCCGIDPGHQFVMALDNGHTIEAAQPLKRGLKRLRKMQQQLSRKYRMNGGKTKNWEKLN
NKIAKLHEKIARHRRSFNHWHSTNLINWFDVIFVEDYKPANVYRKAKAKAKLDTEGAPMVAANGTAIYEKNNQKRKRGS
NRTGNDVAIGQAIDLLETKGKEHGKLVIRVDNWGTTLCCAKCGHQEKKKLSQRTHKCSNCGYTVARDVNSGQNIKLKGL
AQMAINQGVELSDNFWYKFLIDKNSATPSGSKKATKKKPKKSDTVIIPDDLFKNGVHEFLIGETAKTRAIINEMSLGNF
DRNA
(SEQ ID NO: 654)
>SRR3989431_168870_5|M
[freshwater metagenome]
MSSNFSENVRTKEFKLILSSEQERTLEDWMLVCKWVWNRSLGLIEEFNEWNPYDKLSKSNVPATPLQRYDRKLKQWVRI
EIPDWKMGIERVEKKKGLIHPVAIDENSPIIDSLDSKKSVLYGFLKVFGHQHHKDRIVTYLVRGEERKVNFTDCPAKFI
QGVAHELSKAWEGFLAGRHSRPRFKTAKDKVMTLIHYNAKDLGVKDSKINIPKLGYIEVIGFDKRWYGCDFNPMKICKK
ASGWYIQLTASVPVKQAKKTGLCCGIDPGHQFVMALDNGHTIEAAQPLKRSLKRLRKMQQQLSRKYRMNEGKTKNWEKL
NNKIAKLHEKIARHRRSFNHWHSTNLINWFDVIFVEDYKPANVYRKAKAKAKLDTEGNPVVAENGTVIYDKNSQKRKRG
SNRTGSDVAIGQAIDLLETKAKEHGKLVIRVDNWGTTLCCAKCGHQEKKKLSQRTHKCSNCGYTVARDVNSGQNIKLKG
LAQMAINQGVELSDNFWYKFLINKNSATPSCSKKATKKKTKKSDTITIPDDLFKNGVHDFLVAETTKTHAIINQMSLGD
FDRNA
(SEQ ID NO: 655)
>SRR4754862_508403_7|P
[freshwater metagenome]
MSNFSDNVRTKEFKLILSSEQERVLEDWMLVCKWVWNRSLGLIEEFNEWNPYDKISKSNVPATPLQRYDRKLKQWVRIE
IPDWKMGIERVEKKRGFIHPVAIDENSPVIDSLDSKKSVLYGFLKMFGHQHHKDRIVTYLIRGEERKVNFTDCPAKFIQ
GVAHELSKAWEGFLAGRHSRPRFKTAKDKVMTLIHYNAKDLGVKDGKINIPKLGYIEVIGLDKRWYGCDFNPMKICKKA
SGWYMQLTASVPVKQAKKTGLCCGIDPGHQFVMALDNGHTIEAAQPLKRGLKRLRKMQQQLSRKYRMNGGKTKNWEKLN
NKIAKLHEKIARHRRSFNHWHSTNLINWFDVIFVEDYKPANVYRKAKAKAKLDTDGAPMVAANGTAIYEKNNQKRKRGS
NRTGNDVAIGQAIDLLETKGKEHGKLVIRVDNWGTTLCCAKCGHQEKKKLSQRTHKCSNCGYTVARDVNSGQNIKLKGL
AQMAINQGVELSDNFWYKFLIDKNSATPSGSKKATKKKPKKLDTVIIPDDLFKNGVHEFLIGETAKTRAIINEMSLGNF
DRNA
(SEQ ID NO: 656)
>SRR5468113_274154_3|M
[freshwater metagenome]
MNTITKVRKIEFKVTLDAQQRQVFEHHSRDLRFIWNTGLACIQWREWWEKWQQVLESPDRVGYEPETVPINWACNIVKS
PKKKKKDEESEKKVYALACDRVQYRLEKTTNLELGTGEWTEPRKDRNGKIDEDHCWVAYPCKPEPVREHWTEEPRLLLL
MKNPYMALRTMFAKKNWDPETVEGKIIHGLNTAWVAGECKALGDAWSSYKSGVRKRPRFKKRHDRSNLINPSSKQAKFS
GNSLTLPGLGRVTVKGLQQRWGEKPACPIALIKEGDEFYLQLTGELEIPVVRSNKVTIGIDPGSVRLATTDNGAVIEAH
KFGKQMTRRQKKLQASINRKLRLNSDRVYDESDSSRFYLKPRKNWKRKNLQKVRTKLQKLSAKTARQRRAYNHFHSTRI
VQSADVIIMEGLELKNVTRAVKKGKSGEHNKRKAKAGLNRSLLDNAIGQFYGMIEQKAKDAGKVTARVNPFRTSMTCNK
CGFSDAKNRKTQAGFHCQKCGHSDNADRNAALNIKRMGVLGVTKLTFDNDWNITVPGWIEPGDLVALHSKGLKPVGTAK
KRSKRAGKEHPSQLALEIGSQ
(SEQ ID NO: 657)
>SRR5468149_1084624_1|P
[freshwater metagenome]
MKTIKIKLSITPSQSQLIEGYFSELSWIWNKILASEVNNHATQWYKWAAKQSGKCWPAFSLDGANMTPLRFGKSAFIGA
ACQIAIGGAYWKRDDSINVAIKVKGKTEFRKGGKWMKGDRPYTPVPITDYQPRTFTDGTPLLKPADWDRVGKLNQLREA
DGLPPLSLPSDYIGGLVAHFDTAWKAWLDPKLKSRGKLKFKNPREGFVESLSNPQKPPSIDFENETIRIPGIGAIAVGD
RSWRDRIPSGGVPRTYTVNSKPSGWYINIVFANALQPSLKKMEKAAAAVKSQYGKESPEYLEARASVSAIASQISEAGF
VDRKPLSIGIDPGVNAIIGTDHGALFRPNLARERASVRVEYLSAKLATVKELNDKQWKDAGSKRPDTKNEIRLKYQIAR
THEKGANSSNAFNHKLSTRLVGTYTAIAWEDTQLTNLLKQATPKASEEGVGYEKNGAAAKRGLNWSLKQRCLGDLKAKT
KQKMGVLGGEFIDSPAPYSSRECSCCGAVGDRTEQHEFICKNELCEAYLIPKQADVNAARNHAKNAGFSISDVIHSADK
ILYSRPTRRRKKVILLADNRSLGGIDRKVLTRKAQVPGVKEKATTLIDAPELWGTIPKKA
(SEQ ID NO: 658)
>SRR5574091_1575611_4|M
[freshwater metagenome]
MENMTRVVKFRLTLTEEQQSLIDEKMRLLKYLWNLAVGLQEEVNQRRLRDKFEFKEFSPEFWETSYLSLYSSFPHIEKL
PKDKTKLLVGNPCCKIAYLKNKKEYVLLKLEERKKRPYLGNPDAKKHTTKPPDKEALKKYFYPDLADKIFEIGSEFRGC
FYRYVISKAFETAKKGIRGFPKYKGKLDSVQSLIVGHPTLIKIRDSGIFLNKQIGLLVIDDINRLSKESDVRMASIVRK
ASGYYLHLTINLPVPMHFPSDRQIGIDVGCVNLYTDSEGRHIETKAYYRNKERKIAHLQRKMARQEKGSANYLKSKEKL
ALLHEQIKDRRAGYLHQVSHKLTQKYSHIAMEDLKLKNMTKRAGVKENEDGTYAHNGASAKSGLNKSILDQGIGELKRQ
LIYKAEEKGGRVVLVNPKYTSQTCFNCGHKHKDNRKSQSEFICVSCGYSANADENAAKNILTLSLISSTIGVLRSSLLS
YPSLLGKAVSLLAYVCGSPKGTCEAERERSDPPINPPVKGGNCLDNFSTLTIKDPRHQSEKVSQPIDSVQLTIW
(SEQ ID NO: 659)
>SRR6475630_1852385_2|M
[freshwater metagenome]
MSNFSDNVRTKEFKLILSSEQERVLEDWMLVCKWVWNRSLGLIEEFNEWNPYDKISKSNVPATPLQRYDRKLKQWVRIE
IPDWKMGIERVEKKRGFIHPVAIDENSPVIDSLDSKKSVLYGFLKMFGHQHHKDRIVTYLIRGEERKVNFTDCPAKFIQ
GVAHELSKAWEGFLAGRHSRPRFKTAKDKVMTLIHYNAKDLGVKDGKINIPKLGYIEVIGLDKRWYGCDFNPMKICKKA
SGWYMQLTASVPVKQAKKTGLCCGIDPGHQFVMALDNGHTIEAAQPLKRGLKRLRKMQQQLSRKYRMNGGKTKNWEKLN
NKIAKLHEKIARHRRSFNHWHSTNLINWFDVIFVEDYKPANVYRKAKAKAKLDTDGAPMVAANGTAIYEKNNQKRKRGS
NRTGNDVAIGQAIDLLETKGKEHGKLVIRVDNWGTTLCCAKCGHQEKKKLSQRTHKCSNCGYTVARDVNSGQNIKLKGL
AQMAINQGVELSDNFWYKFLIDKNSATPSGSKKATKKKPKKSDTVIIPDDLFKNGVHEFLIGETAKTRAIINEMSLGNF
DRNA
(SEQ ID NO: 660)
>SRR6475633_3251865_1|M
[freshwater metagenome]
MERVEKKRGFIHPVAIDENSPVIDSLDSKKSVLYGFLKMFGHQHHKDRIVTYLIRGEERKVNFTDCPAKFIQGVAHELS
KAWEGFLAGRHSRPRFKTAKDKVMTLIHYNAKDLGVKDGKINIPKLGYIEVIGLDKRWYGCDFNPMKICKKASGWYMQL
TASVPVKQAKKTGLCCGIDPGHQFVMALDNGHTIEAAQPLKRLKRLRKMQQQLSGRKYRMNGGKTKNWEKLNNKIAKLH
EKIARHRRSFNHWHSTNLINWFDVIFVEDYKPANVYRKAKAKAKLDTDGAPMVAANGTAIYEKNNQKRKRGSNRTGNDV
AIGQAIDLLETKGKEHGKLVIRVDNWGTTLCCAKCGHQEKKKLSQRTHKCSNCGYTVARDVNSGQNIKLKGLAQMAINQ
GVELSDNFWYKFLIDKNSATPSGSKKATKKKPKKSDTVIIPDDLFKNGVHEFLIGETAKTRAIINEMSLGNFDRNA
(SEQ ID NO: 661)
>3300031784|Ga0315899_10084352_2|M
[fungi-freshwater]
MPKAKPTDKEKDKRPIKTLEFKLYPNQSQVLTLTDWLDRGRKVWNNGLAVLLEKDQQDWRKKVGFDPMDGNEAWQWFPN
QVGGKTVFGLCCAITFYNRRQNEHYPACFIRHPQNVDGAPRSLIYRATQLDNFCTRFKGGIGDDLLESWKAYKDKNRPT
AKLPKFKGKRFPLKSLSNGECTAKIVGKTAIKFPLLGNIRTKGLERVPQDSKICTARICRKASGWYLQLAIRWDWPVPN
VKMPDVAVAIDPGVRFATSTDYGRQVDAPQFMLKQTKKLRREQRKLSRRKLKGKNWQKQAAKIARLHEKVSRQRNAFWH
KESTYFVTSFGGVAIEDNNFNNMGRKAKAKPKEDGKGYERNNQAQKRGLNRSLKDVACSRLKVMVETKAKTINNEVHLV
RSHHNSQTCSECGHVAKDNRKSQSNFSCVNCGHSLNADINAAINIYNRADWSNRYNLHPRRLQVSDMERYPSFDGGIDA
SQKPATVHGKPSNGTRSQQEGESSPLSEITEKSRSHGLAKPVQLSLWDIVA
(SEQ ID NO: 662)
>3300031857|Ga0315909_10035026_2|M
[fungi-freshwater]
MENMTRVVKFRLTLTENQQSLIDEKMRLLKYLWNLAVGLQEEVNQRRLRDKFEFKEFSPEFWETSYLSLYSSFPHIEKL
PKDKIKLLAGNPCCKIAYLKNKKEYVLLKLEERKKRPYLGNPDAKKHTTKPPDKEDLKKYFYPDLADKIFEIGSEFRGC
FYRYVISKAFETAKKGIRGFPKYKGKLDSVQSLIVGHPTLIKVRDGGIFLNKQIGLLVIDDINRLPKESDVRMASIVRK
ASGYYLHLTINLPVPMHFPSDRQIGIDVGCVNLYTDSEGRHIETKAYYRNKERKIAHLQRKMARQEKGSANYLKSKEKL
ALLHEQIKDRRAGYLHQVSHKLTQKYSHIAMEDLKLKNMTKRAGVKENEDGTYAHNGASAKSGLNKSILDQGIGELKRQ
LIYKSQEKGGRVVLVNPKFTSQTCFNCGHKHKDNRADENAAKNILTLSLISSTIGVLRSSLLSYPSLLGKSQSEFICVS
CGYSANKAVSLLAYVCGSPKGTCEAERERSDPPINPPVKGGNCLDNFSTLTIKDPRHQSEKVSQPIDSVQLTIW
(SEQ ID NO: 663)
>3300032092|Ga0315905_10155087_1|P
[fungi-freshwater]
MSQENITRVVKFRLTLTENQQSLIDEKMRLLKYLWNLAVALQEEVNQRRLRDKFGFKEFSPEFWELSYLKLYSDFSHIE
KLPKDKTKLLVGNPCCKIAYLKNKKDYTPIKLEERKKRPYLGNPDAKKHTAKPPDKKDLKKYFYPDLADKIFEIGSEFR
GCFYRYVISKAFETAKKGIRGFPKYKGKLDSVESLIVGHPTLIKIRDGGIFLNKQIGLLVIDDINRLPKDSDVRMASIV
RKASGYYLHLTVNLPVPMHFPSDRQIGIDVGCVNLYTDSEGRHIETKAYYRNKERKIAHLQRKMARQEKGSANYLKSKE
KLALLHEQIKDRRAGYLHQVSHKLTQKYSHIAMEDLKLKNMTKRANVKENEDGTYAHNGASAKSGLNKSILDQGIGELK
RQLLYKSQEKGGQVVLVNPKFTSQTCFECGHKHKDNRKSQSEFICVSCGHSANADENAAKNILTLSLISSTIGVLRSSL
LSYPSLLGKAVSLLAYVCGSPKGTREAERERSDPPIDPPAKGGNCLDNFSTLTIKDPRHQSEKASQPIDSVQLTLF
(SEQ ID NO: 664)
>3300032092|Ga0315905_10155087_2|M
[fungi-freshwater]
MIQTNIEDKMSQENITRVVKFRLTLTENQQSLIDEKMRLLKYLWNLAVALQEEVNQRRLRDKFGFKEFSPEFWELSYLK
LYSDFSHIEKLPKDKTKLLVGNPCCKIAYLKNKKDYTPIKLEERKKRPYLGNPDAKKHTAKPPDKKDLKKYFYPDLADK
IFEIGSEFRGCFYRYVISKAFETAKKGIRGFPKYKGKLDSVESLIVGHPTLIKIRDGGIFLNKQIGLLVIDDINRLPKD
SDVRMASIVRKASGYYLHLTVNLPVPMHFPSDRQIGIDVGCVNLYTDSEGRHIETKAYYRNKERKIAHLQRKMARQEKG
SANYLKSKEKLALLHEQIKDRRAGYLHQVSHKLTQKYSHIAMEDLKLKNMTKRANVKENEDGTYAHNGASAKSGLNKSI
LDQGIGELKRQLLYKSQEKGGQVVLVNPKFTSQTCFECGHKHKDNRKSQSEFICVSCGHSANADENAAKNILTLSLISS
TIGVLRSSLLSYPSLLGKAVSLLAYVCGSPKGTREAERERSDPPIDPPAKGGNCLDNFSTLTIKDPRHQSEKASQPIDS
VQLTLF
(SEQ ID NO: 665)
>SRR6476078_1283286_3|P
[lake water metagenome]
MIFEPNVKVLEFKIHPTEEQVSKIDQSLAACKLLWNLSIALKEESKQRYYRKKHKFDEFSPEIWELSYSGHYDEKEFKS
LKDKEKKLLIGNPCCKIAYFKKTSNGKEYTPLDAIPIRRFMNAENIDKDAVNYLNRKKLAFYFRENTAKFIGEIETEFK
KGFFKSVIKPAYDAAKKGIRGIPRFKGRRDKVETLVNGQPGTIKIKSNGVIVSSKIGLLKVRGLDRLQGKAPRMAKITR
KATGYYLQLTVETDDTIYKESDKCVGLDMGAVAIFTDDLGRQSEAKRYAKIQKKRLDRLQRQASRQKDGSNNQRKTYAK
LARVHEKIARQRKGRNAQLAHKITSEYQSVILEDLKLKNMTAAAKPKEREDGDGYKQNGKKRKSGLNKALLEMSV
(SEQ ID NO: 666)
>SRR6476078_1283286_3|M
[lake water metagenome]
MKVLEFKIHPTEEQVSKIDQSLAACKLLWNLSIALKEESKQRYYRKKHKFDEFSPEIWELSYSGHYDEKEFKSLKDKEK
KLLIGNPCCKIAYFKKTSNGKEYTPLDAIPIRRFMNAENIDKDAVNYLNRKKLAFYFRENTAKFIGEIETEFKKGFFKS
VIKPAYDAAKKGIRGIPRFKGRRDKVETLVNGQPGTIKIKSNGVIVSSKIGLLKVRGLDRLQGKAPRMAKITRKATGYY
LQLTVETDDTIYKESDKCVGLDMGAVAIFTDDLGRQSEAKRYAKIQKKRLDRLQRQASRQKDGSNNQRKTYAKLARVHE
KIARQRKGRNAQLAHKITSEYQSVILEDLKLKNMTAAAKPKEREDGDGYKQNGKKRKSGLNKALLEMSV
(SEQ ID NO: 667)
>SRR7812565_865334_1|P
[lake water metagenome]
MKTIKIKIKLTTDQVQLCDRYLEELTWLWNLTLSNQLHNHCVTWYAWAAKLSADLDKATEKLDKLKPEQQQLIKDYYRT
KDKPKLSKKEQELVAKFDIFARWNSFSLDGIIPVPLRLGNSGYEGLSCQIATGGNYWKRDENINIPINTKKGIVHVKGY
KLVKGDKPWQRIEIVPHKYRTFPGGKFEGRELTTLEKLDNVNGLNTLRAFQNLPDLQVSSHYIGGLLAFFKESWSAFLD
PKRMNSRKPKFKKDSDKITTLSNNQCAPNRIDVNKNIVTVTGFSPIAIIDKNWVKRLNLSEVLPRTYMLTQNPSGYYIN
IVIAHPLHEEKTALVKKLPKVKKEFGEDSQEYEDIKSKIKFLEQQIKEASIVKGKDLSVGIDPGVQAVVSTDHGALFLP
NLTRERVSIHIEELQSRLDNIELINDKKWKSLGNKTPRIKTKNETKLQEKISRLHERGANSSNAFNHKLSTRLSRTYEH
IAWEDTQINNLLKQVEPKALPEGIGYAHNGASAKRGLNWIMRQRCLSDLKAKTKQKTENRGGNFHEPPANYSSQTCHCC
KQKGERRSQHEFICKNSDCKLFDISQQADTNAARNHKQNGGFDLGDVKYHNVKLVYQKPKRFKKKRLTNQ
(SEQ ID NO: 668)
>SRR6448207_482583_3|P
[microbial mat metagenome]
MKTIKIKLSITPNQSQLIESYFSELSWIWNKILASELSNHATQWYKWAAKQSGKGWPVFSLDGANMTPLRFGKSAFIG
AACQIAIGGAYWKRDDSINVAIKVKGKIEFRKGGKWMKGDRPYTPVPITDYQPRTFTDGTPLLKPADWDRVGKLNQLR
ETDGLPPLSLPSAYIGGLVAHFDAAWKAWLDPKLKSRGKLKFKNPENGFVASLFDRYPPTCIDLENETIRIPGIGAIA
VGDRSWKDRIPSGGVPRTYTVNSKPSGWYINIVFANALQPSLKKMEKAAAAVKSQYGKESPEYLEARASVNAIAFQIS
EDGFVDRKPLSIGIDPGVNAIIGTDHGALFRPNLARERASVRVEYLSAKLDAIKELNDKQWKDAGSKRPDTKNEIKLK
YQIARTHEKGANSSNAFNHKLSTRLVGTYTAIAWEDTQLTNLLKQATPKASEEGVGYEKNGAASKRGLNWSLRQRCLG
DLKAKTKQKMGVLGGEFIDSPAPYSSRECSCCGAVGDRTEQHEFICKNELCTAYLIPKQADVNAARNHAKNAGFSISD
VIHSADKLIYSRPTRRRKKVVSLADNRSLGGIDRKVLTRKAQAPGVKEKATTLIDAPELWGTIPKKA
(SEQ ID NO: 669)
>SRR7244345_343276_44|P
[microbial mat metagenome]
MLTLEFKLSLTTCQEQRLEAWLRDMNWIHNSALACIESFNAHNAYNKEDKRSYACNPVVSYWRGQVYPSCPVGHQLTQF
DEESYAAALLERKNKKGTVYPLQAWAGEPRLENVSYFSLLNRFPKKLHPIKLADVPQKFVAGRVERVALSWQAFLKGNA
KPPKFKSRRNPVTSLIHNESKKIRIDGDRINMPRIGWLKAKGLAKRWPQGVPFCPMKVIKAADGWYLQLTGEVEERKSP
KLTGRHVGIDPGSVRHHTTDSGSFVEPPRYLMKAAVKLRTLQRQLSRQYRQNSTQIFGKDGRVIRSVARSDWDRKNFDK
TKAKIAKLHAKIARQRRAFNHFQSSKYVTMFDVITVEDFEPAKLTRAVKKGEAGVRNGRKAKSGLNRNLLDNAIGQFYG
MLEQKSKDRGRAFQRVKPAFSSRTCNCCGYRSKDNRKSQSSFVCLSCGHRDNADRNAARNIKRMAELGIDRLTFTDDFA
DGFREQVGVDGAKDVVSLPKKAKKRGSRKSTMEVSCQLTLDEGSSNSL
(SEQ ID NO: 670)
>WP_016456110.1
[Propionimicrobium lymphophilum ACS-093-V-SCH5]
MSYLCARGRFNLSGFSTGSMGVLPVRASLFALLFTTLHGARISAHWYGMSKENDHTVTCIKICLEPNKAQRAQFASFAG
SARWAYNFALAIKIGYQKRWFEARKQFIESGLDEKAAGKKASEQVGRMPNYMSIATNEWTQLRDEVCPWYPEVPRRVFV
GGFQRADAAFKNWFDSKSGRRSGAAMGWPKFKSKSKSRESFVIANDVQPAFVANLNRYIKTGELADMDYRHIKVPKCGE
VRLTPGSAGQLRQLGRTMLAEAKTGELITRITSGTISRLGDRWYVSLVISGPFVPDAISTKRQRRNGVVGVDLGSGRFY
ATTSEGLSIINPKFVSKYEQELARANRALAKTAKGSAARKKALARLRRVHARSALARDGFSHQVSAWLTSQFAGVAVEK
FDLASMLASAKGTVEKPGKNVDVKARFNAHLADVGIASTIDKLLYKGKRDGCRVQVVNTLDNSSTTCAKCGHTCVCGPE
QKTFTCPDCGYNAPRQLNSAQYIRQLATVGFDELGLDMTASLTPDTGKRPIAFMTSAH
(SEQ ID NO: 671)
>AGZR01000008_18|M
[Propionimicrobium lymphophilum ACS-093-V-SCH5]
MGVLPVRASLFALLFTTLHGARISAHWYGMSKENDHTVTCIKICLEPNKAQRAQFASFAGSARWAYNFALAIKIGYQKR
WFEARKQFIESGLDEKAAGKKASEQVGRMPNYMSIATNEWTQLRDEVCPWYPEVPRRVFVGGFQRADAAFKNWFDSKSG
RRSGAAMGWPKFKSKSKSRESFVIANDVQPAFVANLNRYIKTGELADMDYRHIKVPKCGEVRLTPGSAGQLRQLGRTML
AEAKTGELITRITSGTISRLGDRWYVSLVISGPFVPDAISTKRQRRNGVVGVDLGSGRFYATTSEGLSIINPKFVSKYE
QELARANRALAKTAKGSAARKKALARLRRVHARSALARDGFSHQVSAWLTSQFAGVAVEKFDLASMLASAKGTVEKPGK
NVDVKARFNAHLADVGIASTIDKLLYKGKRDGCRVQVVNTLDNSSTTCAKCGHTCVCGPEQKTFTCPDCGYNAPRQLNS
AQYIRQLATVGFDELGLDMTASLTPDTGKRPIAFMTSAH
(SEQ ID NO: 672)
>SRR8162779_1368970_53|P
[sediment metagenome]
MLTLEFKLSLTISQEQRLEAWLRDMSWVHNSALACIESFNAHNAYHKGDKRSYACNPVVSYWRGQVYPSCPVSHQLTQF
DEESYAAALRERKNKKGTVYPLQAWAGEPVFGQISPFSLQARFGINRHPLKLKAVGIPKVFVAGRTNIVAKSWDAFCNR
ASKPPKFKSRRNPVQSLISINSQATSIEGDRIRIPNLGFAKAKGLSKRWPTGAVVSTLKIIKAADGWYLQLTGEVGERK
PPKLTGRHVGIDPGAVRHHTTDSGSFVEPPRYLLKAAQKLRMLQRQLSRQYRQNSTQIFGKDGRVIRSVARSDWDRKNF
DKTKAKIAKLHAKIARQRRAFNHFQSSKYAAMFDVITVEDFEPAKLTRAVKKGEAGVRNGRKAKSGLNRNLLDNAIGQF
YGMLEQKSKDRGRLFQRVKPAFSSQTCNCCGYRSKDNRQSQSSFVCLCGHRDNADRNAARNIKRMAESLGIDRLTFTDD
FADGFRERVGVDGAKDVVSLPKKAKKRGSRKSTMEVPCQLTLDEGMFK
(SEQ ID NO: 673)
>SRR5855413_300079_4|M
[soil crust metagenome]
MKTLEFKLYLTRSQQETIDNWLSALKYPWNKGVELIHTFNDFNYYDKRFKTLAPCCPIASYNHKLRIVNAKNREKKQQL
RENEYLSCPIGWKLDDYTGFSTSLETRELMQVLVDEGKTLEKVGTRSPYGIYPIRPFASGKLVTNLSYFELLSVFAHKR
NVDRPWFIDADEPWFTDVDSKIIAGMVKTLADSWQAFLDGKRKPPKFKGKNKRIDTLINNNSKDIRVEGDRIKLPKIGF
VRAKGLAKRWNQGLVFCPMKICRKASGYYLQLTGEVPPEPLKKHPKLKAVGLDPGIAAVYTDDAGRTVKVPPYLEKDLG
KLKSLQRKVQRQWENNKDIPEWERKNWQKTQNKIAKLHEKIARRGRAFNHFESTKLVRQFEEIYLEDYRPSEMVGKVKP
IDSGKVVVNTKDELTTVYEKNGRGVNKLINRATLRNRVGQLWQMIETKGGKRVLRVERNGTSSECPKCGDIKEKLIQQR
IHRCGECGYITHRDRAAAQIIKQRGLEIRQAGGDEDNGKKQRQRRDRKKG
(SEQ ID NO: 674)
>SRR5855413_1311040_12|M
[soil crust metagenome]
MKTLEFKLYLTDSQQQIVDEWLAALKFVWNKGVELMEDFKAFNHYDKRFKATAPCCPIVSYERKLRQVLPTCPVGWRLD
DYSGLYHTKNEQGEWELVQVVMDEDKLLDKLVEKRSPNGIFPMRPWRNGKMLTGITHYGLEPIFAHKRNTDKPWLTAVD
SKFISGMVKSLADGWTAFLGGSRDRPPKFKNKSVRINTLIHNNTKPDKSGRAVAPISGDRIRIPKIGWVRVKGLSKRWP
KGLEFCPLKICRKASGYYLQLTGDVKNDILIKPPSVQAIGLDPGIEAVFTDDVGRRIKVPDYLERDLKKLKQLQRKASR
QWEANKECEEWHRKNWRKTQDKIAKLHEKIARRGRAFNHFQSTKLVRIFNEIYLEDYRPSEMVGKVEPVDSGKLVINKN
EDLTTVYEKNGREVNRAINRATLRNRVGQLWQMIETKGGKRVLRVERDGTSSECPRCGYVKQKRIQERTHRCEQCGYKA
HRDQAAAEVIKKKGQEARLKGCVEQENTKQRRKGRGKKVSGE
(SEQ ID NO: 675)
>SRR5855415_346708_l05|P
[soil crust metagenome]
MIDRQGRQQCRLKLMKTIEFKLSLTRSQEEILNQWMSALKFVWNLGVALIKEFNSFNHYDKRFKQSAPCCPIVAYNHKL
RVTNGATQEGWRECEFPIQPIGFQLLAFDEENPIDVGKRTPCGVYPVIVNGHAVKNLTYFGIQGAFAHKRNQDKPWLTC
IPSKFVHGTVQSLADSWQAFLSGNSKRPKFKGKNDQIKTLVTNNAKDITTIKGDRIRIPNLGYVRAKGLSKRWHEGVPI
CSLKICKKASGYYLQLTGDIEACSPKKTKIPKVQAVGLDPGIEHVYCDDAGRKVKVPQYLERDLQKLKQLQRKAQKQWD
MNKDTVDQNGEPWQKKNWRRTQDKIAKLHERIARRGRAFNHFQSTKLVRQFQQIYLEDYKPSEVIKKVAPVDSGKMVVN
KNDELTTIYEKNERFLNRSTNRAAQRNRVGQLWTMIKTKGGKKVVLVEAAGTSDECPSCGHVEEKTLRQRLHRCKNCGF
VAHRDVAAAKIIKKRGMAQSPQMDVSLR
(SEQ ID NO: 676)
>SRR5855420_1331858_67|P
[soil crust metagenome]
MYLTDSQQQ1VDEWLAALKFVWNKGVELMEDFKAFNHYDKRFKATAPCCPIVSYERKLRQVLPTCPVGWRLDDYSGLYH
TKNEQGEWELVQVVMDEDKLLDKLVEKRSPNGIFPMRPWRNGKMLTGITHYGLEPIFAHKRNTDKPWLTAVDSKFISGM
VKSLADGWTAFLGGSRDRPPKFKNKSVRINTLIHNNTKPDKSGRAVAPISGDRIRIPKIGWVRVKGLSKRWPKGLEFCP
LKICRKASGYYLQLTGDVKNDILIKPPSVQAIGLDPGIEAVFTDDVGRRIKVPDYLERDLKKLKQLQRKASRQWEANKE
CEEWHRKNWRKTQDKIAKLHEKIARRGRAFNHFQSTKLVRIFNEIYLEDYRPSEMVGKVEPVDSGKLVINKNEDLTTVY
EKNGREVNRAINRATLRNRVGQLWQMIETKGGKRVLRVERDGTSSECPRCGYVKQKRIQERTHRCEQCGYKAHRDQAAA
EVIKKKGQEARLKGCVEQENTKQRRKGRGKKVSGE
(SEQ ID NO: 677)
>SRR5855420_1331858_68|M
[soil crust metagenome]
MKTLEFKLYLTDSQQQIVDEWLAALKFVWNKGVELMEDFKAFNHYDKRFKATAPCCPIVSYERKLRQVLPTCPVGWRLD
DYSGLYHTKNEQGEWELVQVVMDEDKLLDKLVEKRSPNGIFPMRPWRNGKMLTGITHYGLEPIFAHKRNTDKPWLTAVD
SKFISGMVKSLADGWTAFLGGSRDRPPKFKNKSVRINTLIHNNTKPDKSGRAVAPISGDRIRIPKIGWVRVKGLSKRWP
KGLEFCPLKICRKASGYYLQLTGDVKNDILIKPPSVQAIGLDPGIEAVFTDDVGRRIKVPDYLERDLKKLKQLQRKASR
QWEANKECEEWHRKNWRKTQDKIAKLHEKIARRGRAFNHFQSTKLVRIFNEIYLEDYRPSEMVGKVEPVDSGKLVINKN
EDLTTVYEKNGREVNRAINRATLRNRVGQLWQMIETKGGKRVLRVERDGTSSECPRCGYVKQKRIQERTHRCEQCGYKA
HRDQAAAEVIKKKGQEARLKGCVEQENTKQRRKGRGKKVSGE
(SEQ ID NO: 678)
>SRR5855435_639752_6|P
[soil crust metagenome]
MKTLEFKLYLTRSQQETIDNWLSALKYPWNKGVELIHTFNDFNYYDKRFKTLAPCCPIASYNHKLRIVNAKNREKKQQL
RENEYLSCPIGWKLDDYTGFSTSLETRELMQVLVDEGKTLEKVGTRSPYGIYPIRPFASGKLVTNLSYFELLSVFAHKR
NVDRPWFIDADEPWFTDVDSKIIAGMVKTLADSWQAFLDGKRKPPKFKGKNKRIDTLINNNSKDIRVEGDRIKLPKIGF
VRAKGLAKRWNQGLVFCPMKICRKASGYYLQLTGEVPPEPLKKHPKLKAVGLDPGIAAVYTDDAGRTVKVPPYLEKDLG
KLKSLQRKVQRQWENNKDIPEWERKNWQKTQNKIAKLHEKIARRGRAFNHFESTKLVRQFEEIYLEDYRPSEMVGKVKP
IDSGKVVVNTKDELTTVYEKNGRGVNKLINRATLRNRVGQLWQMIETKGGKRVLRVERNGTSSECPKCGDIKEKLIQQR
IHRCGECGYITHRDRAAAQIIKQRGLEIRQAGGDEDNGKKQRQRRDRKKG
(SEQ ID NO: 679)
>SRR6201738_878635_2|M
[soil metagenome]
MDKSKRQGEVAEKQLRESLPQKRSIRGAFLFDKMKACQAEAQRIVKMLTLEFSVEFTPEQESIISQWMDLQKIQWNIGL
SALLEFDATRRWNKLTDSDGGKGAYYPVCAIAGGDWVPASAWGYEAIPRNDGQEPLKGWTARACPLYKVGVGDRWLSGG
CTIPEIGAMVPNVEPLLLSIKDAKSPGGLSIMARADNIPSDLAETPSLFRHGTLALLFASWDRHKSDPLNAGQPKRKRY
QDKWDVLYTGNAKELSKTSDAVRIPKIGWVGIPKLSDRWRDSEGNAPDICSFKIVRRDRKFRVQLTGDLKRSYKAQPSN
LSVGVDLGFVYAHIESNGNRSALLDLSLEKLEQRKQRLQAKLDSKLDQRIVLWLRNPETTHASLIVRRKTRRGIEEEKP
MIRISLENWEKLKQCRTAGEAAQIIGQKRDKRYQTLRHALPKSAKEQALRVQIASIDRKIAATRKTRDAKLATRLAKKY
GHIAIENGLQREVKRHRPDPKKQGDKYLKNGAERQSETNKQLKALAPGRKIAMLEAMSKRYGRDFSKQESPTTTTECPC
CGRHNEPSLKMDEHGDRRYSCACGWEIDQDVNAATNIELRSSALKPEVQLSQPAETARIRSYQLEADGKLFAEPQWRMP
PAPPKAKKESRGKRRKALRDSEPLKELKGQRLTAASRKSQVKALESGVSKMLP
(SEQ ID NO: 680)
>SRR6201739_286584_1|P
[soil metagenome]
MDKSKRQGEVAEKQLRESLPQKRSIRGAFLFDKMKACQAEAQRIVKMLTLEFSVEFTPEQESIISQWMDLQKIQWNIGL
SALLEFDATRRWNKLTDSDGGKGAYYPVCAIAGGDWVPASAWGYEAIPRNDGQEPLKGWTARACPLYKVGVGDRWLSGG
CTIPEIGAMVPNVEPLLLSIKDAKSPGGLSIMARADNIPSDLAETPSLFRHGTLALLFASWDRHKSDPLNAGQPKRKRY
QDKWDVLYTGNAKELSKTSDAVRIPKIGWVGIPKLSDRWRDSEGNAPDICSFKIVRRDRKFRVQLTGDLKRSYKAQPSN
LSVGVDLGFVYAHIESNGNRSALLDLSLEKLEQRKQRLQAKLDSKLDQRIVLWLRNPETTHASLIVRRKTRRGIEEEKP
MIRISLENWEKLKQCRTAGEAAQIIGQKRDKRYQTLRHALPKSAKEQALRVQIASIDRKIAATRKTRDAKLATRLAKKY
GHIAIENGLQREVKRHRPDPKKQGDKYLKNGAERQSETNKQLKALAPGRKIAMLEAMSKRYGRDFYKQESPTTTTECPC
CGRHNEPSLKMDEHGDRRYSCACGWEIDQDVNAATNIELRSSALKPEVQLSQPAETARIRSYQLEADGKLFAEPQWHMP
PAPPKAKKESRGKRRKALRDSEPLKELKGQRLTAASRKSQVKALESGVSKMLP
(SEQ ID NO: 681)
>GCL37732.1
[Sphaerospermopsis reniformis]
MKTIKIKVSLSTQQKEIFNQYIDQLEWFWNLALANQFHNHCIKWYEWAKKQQQLLDKTKENFDKLKPEQKQLVLDFYSH
KYGEKQPKLTEKQKDLINKFEIFSRWLPFDLDGIIPVPLLLGNSGYEGLSCRIAIGGPRWVRDENINIPLKNKKGEIIH
KKGSKLVPGKHPYQPIKPTPHSYKTFPGGKFAGRELVDVKKLDNLDGLNSIRSAENLPPLTVPTDYIGGTMKFFEVSWN
AFLDPKLPERRKVKFKDKERKLTTLSNNQKPPNRINPEKNTVSISGIGEVKVIDRNWLQRLNLENISPRTYTLSKKPSG
YYLNIVVAHPLQEEKSKLDKQLPKVKKEYGEESQEYQEILSKYNEVSEQIKSSYFDDVKDLSVGIDPGVNAVISTDHGA
LFMPNITRERISIHIEELQSKLNKIKDINDAQWKAAGNKGERPKTKNEIKLQAQISRLHEKGANSANCFNHKLSTRIAR
TYRYVCWEDTKIKNLLKQVEPQALPEGVGYAHNGASAKRGLNWIMRQRCLSDLEAKTKEKVEKKGGKFNEPSANYSSQL
CHCCQQKGERVSQHEFICKNPECSLFEKVQQADVNAARNHKQHGGFEVGEVKYNLTKLQYQKPKRFKKKKLTK
(SEQ ID NO: 682)

TABLE 3010
Nucleotide Sequences of Representative CLUST.057059 Direct
Repeats and Spacer Lengths

| CLUST.057059 Effector | | Spacer |
|---|---|---|
| Protein Accession | Direct Repeat Nucleotide Sequence | Length(s) |
| 3300023179\|Ga0214923_10000634_6\|0P | CTTGCAACTGGGCTTGGGGACTGAGGATAGTTGAAAC | 32- |
| (SEQ ID NO: 601) | (SEQ ID NO: 683) | 39 |
| | GTTTCAACTATCCTCAGTCCCCAAGCCCAGTTGCAAG | |
| | (SEQ ID NO: 751) | |
| ATW59329.1 | GTTAGGATGCCCGCAGGATGTCATATAGTTGGCAAG | 26- |
| (SEQ ID NO: 602) | (SEQ ID NO: 684) | 45 |
| | CTTGCCAACTATATGACATCCTGCGGGCATCCTAAC | |
| | (SEQ ID NO: 752) | |
| SRR3727507_43825_27\|P | ATTGCAATAAATTTTAATTCTTAGCAGGGATTGAAAG | 34- |
| (SEQ ID NO: 603) | (SEQ ID NO: 685) | 37 |
| | CTTTCAATCCCTGCTAAGAATTAAAATTTATTGCAAT | |
| | (SEQ ID NO: 753) | |
| SRR3727511_164637_6\|M | ATTGCAATAAATTTTAATTCTTAGCAGGGATTGAAAG | 34- |
| (SEQ ID NO: 604) | (SEQ ID NO: 685) | 36 |
| | CTTTCAATCCCTGCTAAGAATTAAAATTTATTGCAAT | |
| | (SEQ ID NO: 753) | |
| SRR3727516_4364_11\|P | ATTGCAATAAATTTTAATTCTTAGCAGGGATTGAAAG | 34- |
| (SEQ ID NO: 605) | (SEQ ID NO: 685) | 36 |
| | CTTTCAATCCCTGCTAAGAATTAAAATTTATTGCAAT | |
| | (SEQ ID NO: 753) | |
| SRR3727519_259012_1\|M | TGCAATAAATTTTAATTCTTAGCAGGGATTGAAAG | 36- |
| (SEQ ID NO: 606) | (SEQ ID NO: 686) | 39 |
| | CTTTCAATCCCTGCTAAGAATTAAAATTTATTGCA | |
| | (SEQ ID NO: 754) | |
| SRR3727519_13189_1\|M | ATTGCAATAAATTTTAATTCTTAGCAGGGATTGAAAG | 34- |
| (SEQ ID NO: 607) | (SEQ ID NO: 685) | 36 |
| | CTTTCAATCCCTGCTAAGAATTAAAATTTATTGCAAT | |
| | (SEQ ID NO: 753) | |
| SRR5216639_261560_2\|P | CTTGAAATTCCTTTTAATGCCTATCAGGAATTGAAAC | 30- |
| (SEQ ID NO: 608) | (SEQ ID NO: 687) | 49 |
| | GTTTCAATTCCTGATAGGCATTAAAAGGAATTTCAAG | |
| | (SEQ ID NO: 755) | |
| SRR5216639_20646\|03_3\|P | GTTCCCAATAGGGGGACTTCTCGATCCAATAGAAAG | 24- |
| (SEQ ID NO: 609) | (SEQ ID NO: 688) | 38 |
| | CTTTCTATTGGATCGAGAAGTCCCCCTATTGGGAAC | |
| | (SEQ ID NO: 756) | |
| SRR5216639_2018995_2\|M | GTTGCCAAGAGGGCGACTTCTAAGGTTGATGGGAAG | 34- |
| (SEQ ID NO: 610) | (SEQ ID NO: 689) | 44 |
| | CTTCCCATCAACCTTAGAAGTCGCCCTCTTGGCAAC | |
| | (SEQ ID NO: 757) | |
| SRR5216639_409921_41\|M | CTTGCAAGAAGGGCGGAGGGATGAGAGGTGTTGAAAC | 31- |
| (SEQ ID NO: 611) | (SEQ ID NO: 690) | 45 |
| | GTTTCAACACCTCTCATCCCTCCGCCCTTCTTGCAAG | |
| | (SEQ ID NO: 758) | |
| SRR5216639_409921_40\|P | CTTGCAAGAAGGGCGGAGGGATGAGAGGTGTTGAAAC | 31- |
| (SEQ ID NO: 612) | (SEQ ID NO: 690) | 45 |
| | GTTTCAACACCTCTCATCCCTCCGCCCTTCTTGCAAG | |
| | (SEQ ID NO: 758) | |
| SRR8571171_1127661_43\|P | CTTGCAAGAAGGGCGGAGGGACAGGAGGGACTGAAAC | 34- |
| (SEQ ID NO: 613) | (SEQ ID NO: 691) | 44 |
| | GTTTCAGTCCCTCCTGTCCCTCCGCCCTTCTTGCAAG | |
| | (SEQ ID NO: 759) | |
| 3300002370\|release\| | CGCAATAGACGATTGCCTATTTTATGGATTGAAAC | 38 |
| scaffold29556_3\|P | (SEQ ID NO: 692) | |
| (SEQ ID NO: 614) | GTTTCAATCCATAAAATAGGCAATCGTCTATTGCG | |
| | (SEQ ID NO: 760) | |
| 33000023821017448\| | CTTGCAACTAAGACTGGGGACTGAGGATAGTTGAAAC | 32- |
| scaffold9922858_2\| | T (SEQ ID NO: 693) | 48 |
| M (SEQ ID NO: 615) | AGTTTCAACTATCCTCAGTCCCCAGTCTTAGTTGCAA | |
| | G (SEQ ID NO: 761) | |
| 3300002396\|B570J29629_1000007_3\|P | CGCAATAGACGATTGCCTATTTTATGGATTGAAAC | 38 |
| (SEQ ID NO: 616) | (SEQ ID NO: 692) | |
| | GTTTCAATCCATAAAATAGGCAATCGTCTATTGCG | |
| | (SEQ ID NO: 760) | |
| 3300002396\|B570J29629_1000558_1\|M | CTTGCAACTAAGACTGGGGACTGAGGATAGTTGAAAC | 34- |
| (SEQ ID NO: 617) | (SEQ ID NO: 694) | 50 |
| | GTTTCAACTATCCTCAGTCCCCAGTCTTAGTTGCAAG | |
| | (SEQ ID NO: 762) | |
| 3300002408\|1017274\| | CGCAATAGACGATTGCCTATTTTATGGATTGAAAC | 36- |
| scaffold00152_5\|M | (SEQ ID NO: 692) | 38 |
| (SEQ ID NO: 618) | GTTTCAATCCATAAAATAGGCAATCGTCTATTGCG | |
| | (SEQ ID NO: 760) | |
| 3300013005\|Ga0164292_10021448_2\|P | GTTCCAATTAATCTTAAGTCCTATTAGGGATTGAAAC | 34- |
| (SEQ ID NO: 619) | (SEQ ID NO: 695) | 43 |
| | GTTTCAATCCCTAATAGGACTTAAGATTAATTGGAAC | |
| | (SEQ ID NO: 763) | |
| 3300027808\|Ga0209354_10002575_5\|M | CCAACAGATGGCTAGAGTTGTCTAGGCAGTTGAAGG | 36- |
| (SEQ ID NO: 620) | (SEQ ID NO: 696) | 43 |
| | CCTTCAACTGCCTAGACAACTCTAGCCATCTGTTGG | |
| | (SEQ ID NO: 764) | |
| 3300020048\|Ga0207193_1000230_60\|P | CCCGCAATAGACGATTGCCTATTTTATGGATTGAAAC | 34- |
| (SEQ ID NO: 621) | (SEQ ID NO: 697) | 41 |
| | GTTTCAATCCATAAAATAGGCAATCGTCTATTGCGGG | |
| | (SEQ ID NO: 765) | |
| 3300015360\|Ga0163144_10269315_2\|M | CTTGCAATAAACTTTAATCCTTAGTAGGGATTGAAAC | 33- |
| (SEQ ID NO: 622) | (SEQ ID NO: 698) | 38 |
| | GTTTCAATCCCTACTAAGGATTAAAGTTTATTGCAAG | |
| | (SEQ ID NO: 766) | |
| 3300022561\|Ga0212090_10036221_2\|M | TTTCAATCCCTACCAAGGGTT | 26- |
| (SEQ ID NO: 623) | (SEQ ID NO: 699) | 43 |
| | AACCCTTGGTAGGGATTGAAA | |
| | (SEQ ID NO: 767) | |
| 3300005805\|Ga0079957_1000538_44\|M | ATTGCAATATATTTTGATCCTTGGTAGGGATTGAAAG | 23- |
| (SEQ ID NO: 624) | (SEQ ID NO: 700) | 36 |
| | CTTTCAATCCCTACCAAGGATCAAAATATATTGCAAT | |
| | (SEQ ID NO: 768) | |
| 3300005805\|Ga0079957_1019747_2\|M | CTTGCAACTGGGCTTAGGGACTGAGGATAGTTGAAAC | 33- |
| (SEQ ID NO: 625) | (SEQ ID NO: 701) | 42 |
| | GTTTCAACTATCCTCAGTCCCTAAGCCCAGTTGCAAG | |
| | (SEQ ID NO: 769) | |
| 3300001043\|JGI12316J14372_10004500_ | ATGACATCTGGGGGTTGGAAGGGGTTGCAAG | 18- |
| 6\|M | (SEQ ID NO: 702) | 44 |
| (SEQ ID NO: 626) | CTTGCAACCCCTTCCAACCCCCAGATGTCAT | |
| | (SEQ ID NO: 770) | |
| 3300001043JGI12316J15309_10000277_ | ATGACATCTGGGGGTTGGAAGGGGTTGCAAG | 42- |
| 14\|M | (SEQ ID NO: 702) | 44 |
| (SEQ ID NO: 627) | CTTGCAACCCCTTCCAACCCCCAGATGTCAT | |
| | (SEQ ID NO: 770) | |
| 3300001341\|1010336\|scaffold_14_ | ATGACATCTGGGGGTTGGAAGGGGTTGCAAG | 42- |
| 1570\|P | (SEQ ID NO: 702) | 44 |
| (SEQ ID NO: 628) | CTTGCAACCCCTTCCAACCCCCAGATGTCAT | |
| | (SEQ ID NO: 770) | |
| 3300027607\|Ga0207422_1002635_7\|P | ATGACATCTGGGGGTTGGAAGGGGTTGCAAG | 42- |
| (SEQ ID NO: 629) | (SEQ ID NO: 702) | 44 |
| | CTTGCAACCCCTTCCAACCCCCAGATGTCAT | |
| | (SEQ ID NO: 770) | |
| 3300007960\|1Ga0099850_1030516_1\|P | GTTGCCAAGAGGGTGACTAATTGGGTTAAG | 26- |
| (SEQ ID NO: 630) | (SEQ ID NO: 703) | 38 |
| | CTTAACCCAATTAGTCACCCTCTTGGCAAC | |
| | (SEQ ID NO: 771) | |
| 3300028735\|Ga0272446_1017865_2\|M | CTGATAAAGTTTTTGGGTACT | 49- |
| (SEQ ID NO: 631) | (SEQ ID NO: 704) | 58 |
| | AGTACCCAAAAACTTTATCAG | |
| | (SEQ ID NO: 772) | |
| SRR6869054_1535443_5\|P | GTCACCATGACATCTGGAGGTTGGCAGGGGTTGCAAG | 35- |
| (SEQ ID NO: 632) | (SEQ ID NO: 705) | 38 |
| | CTTGCAACCCCTGCCAACCTCCAGATGTCATGGTGAC | |
| | (SEQ ID NO: 773) | |
| SRR6869054_457352_2\|P | GCTGAAACCAAACTAATCCGCTCTTAGGGATTGAAAG | 34- |
| (SEQ ID NO: 633) | (SEQ ID NO: 706) | 37 |
| | CTTTCAATCCCTAAGAGCGGATTAGTTTGGTTTCAGC | |
| | (SEQ ID NO: 774) | |
| SRR6869055_1317318_5\|P | GTCACCATGACATCTGGAGGTTGGCAGGGGTTGCAAG | 35- |
| (SEQ ID NO: 634) | (SEQ ID NO: 705) | 38 |
| | CTTGCAACCCCTGCCAACCTCCAGATGTCATGGTGAC | |
| | (SEQ ID NO: 773) | |
| SRR3137750_862387_23\|M | CGATCAAAGGACTAATCCCGGCGATCGGGACTGAAAC | 35- |
| (SEQ ID NO: 635) | T (SEQ ID NO: 707) | 48 |
| | AGTTTCAGTCCCGATCGCCGGGATTAGTCCTTTGATC | |
| | G (SEQ ID NO: 775) | |
| SRR3138838_558748_1\|P | CCGACAAACGACTAGAGTTGTCTAGGCAGTTGAAGG | 37- |
| (SEQ ID NO: 636) | (SEQ ID NO: 708) | 38 |
| | CCTTCAACTGCCTAGACAACTCTAGTCGTTTGTCGG | |
| | (SEQ ID NO: 776) | |
| SRR3139299_296800_1\|M | CTCGCAATAGACGATTGCCTATTTTATGGATTGAAAC | 34- |
| (SEQ ID NO: 637) | (SEQ ID NO: 709) | 40 |
| | GTTTCAATCCATAAAATAGGCAATCGTCTATTGCGAG | |
| | (SEQ ID NO: 777) | |
| SRR3139690_493772_1\|M | GTCGCAATCTGGCTGGAT (SEQ ID NO: 710) | 53 |
| (SEQ ID NO: 638) | ATCCAGCCAGATTGCGAC (SEQ ID NO: 778) | |
| SRR3139690_185157_1\|M | CTTTCAAGTTAACTCTGTAGAGACGGTTTGAAAG | 35- |
| (SEQ ID NO: 639) | (SEQ ID NO: 711) | 43 |
| | CTTTCAAACCGTCTCTACAGAGTTAACTTGAAAG | |
| | (SEQ ID NO: 779) | |
| SRR3139690_493772_1\|P | GTCGCAATCTGGCTGGAT (SEQ ID NO: 710) | 53 |
| (SEQ ID NO: 640) | ATCCAGCCAGATTGCGAC (SEQ ID NO: 778) | |
| WP_015957291.1 | GTTTCTTATTAATGAATCCTAGCAATGGGATTGAAAC | 33- |
| (SEQ ID NO: 641) | (SEQ ID NO: 712) | 37 |
| | GTTTCAATCCCATTGCTAGGATTCATTAATAAGAAAC | |
| | (SEQ ID NO: 780) | |
| CP001287_4134\|P | GTTTCTTATTAATGAATCCTAGCAATGGGATTGAAAC | 33- |
| (SEQ ID NO: 642) | (SEQ ID NO: 712) | 37 |
| | GTTTCAATCCCATTGCTAGGATTCATTAATAAGAAAC | |
| | (SEQ ID NO: 780) | |
| WP_015785111.1 | GTTTCTTATTAATGAATCCTAGCAATGGGATTGAAAC | 34- |
| (SEQ ID NO: 643) | (SEQ ID NO: 712) | 37 |
| | GTTTCAATCCCATTGCTAGGATTCATTAATAAGAAAC | |
| | (SEQ ID NO: 780) | |
| NC_013161_4122\|P | GTTTCTTATTAATGAATCCTAGCAATGGGATTGAAAC | 34- |
| (SEQ ID NO: 644) | (SEQ ID NO: 712) | 37 |
| | GTTTCAATCCCATTGCTAGGATTCATTAATAAGAAAC | |
| | (SEQ ID NO: 780) | |
| OVJI010000022_17\|P | TGGCGGTACTGGTGGTACTGG | 16- |
| (SEQ ID NO: 645) | (SEQ ID NO: 713) | 22 |
| | CCAGTACCACCAGTACCGCCA | |
| | (SEQ ID NO: 781) | |
| CAAAFE010002307_2\|M | GTAGAAACCAACAGGGTTTCTGGAAAGGGATTGAAAG | 32- |
| (SEQ ID NO: 646) | (SEQ ID NO: 714) | 36 |
| | CTTTCAATCCCTTTCCAGAAACCCTGTTGGTTTCTAC | |
| | (SEQ ID NO: 782) | |
| OGWG01000020_5\|P | GTTCCCATCGTACTGACTTCTCTGGTCGTTCCCGAC | 27- |
| (SEQ ID NO: 647) | (SEQ ID NO: 715) | 40 |
| | GTCGGGAACGACCAGAGAAGTCAGTACGATGGGAAC | |
| | (SEQ ID NO: 783) | |
| OGWG01000020_4\|M | GTTCCCATCGTACTGACTTCTCTGGTCGTTCCCGAC | 27- |
| (SEQ ID NO: 648) | (SEQ ID NO: 715) | 40 |
| | GTCGGGAACGACCAGAGAAGTCAGTACGATGGGAAC | |
| | (SEQ ID NO: 783) | |
| OGWL01001214_2\|P | GTTGCCAACAGGGCGACTTCATTGGTTAATGGGAAG | 19- |
| (SEQ ID NO: 649) | (SEQ ID NO: 716) | 41 |
| | CTTCCCATTAACCAATGAAGTCGCCCTGTTGGCAAC | |
| | (SEQ ID NO: 784) | |
| OGW001000325_8\|P | CTCGAAATTCCTATTAATGCCTATCAGGAATTGAAAC | 32- |
| (SEQ ID NO: 650) | (SEQ ID NO: 717) | 37 |
| | GTTTCAATTCCTGATAGGCATTAATAGGAATTTCGAG | |
| | (SEQ ID NO: 785) | |
| OGWP01000327_5\|M | GTTCCCATCGTACTGACTTCTCTGGTCGTTCCCGAC | 33- |
| (SEQ ID NO: 651) | (SEQ ID NO: 715) | 42 |
| | GTCGGGAACGACCAGAGAAGTCAGTACGATGGGAAC | |
| | (SEQ ID NO: 783) | |
| OGWP01001429_1\|P | CTTTCTATCAGAGTGAGAAGTCCCCCTGTTGGGAACT | 32- |
| (SEQ ID NO: 652) | (SEQ ID NO: 718) | 44 |
| | AGTTCCCAACAGGGGGACTTCTCACTCTGATAGAAAG | |
| | (SEQ ID NO: 786) | |
| OGWP01001429_1\|M | CTTTCTATCAGAGTGAGAAGTCCCCCTGTTGGGAACT | 32- |
| (SEQ ID NO: 653) | (SEQ ID NO: 718) | 44 |
| | AGTTCCCAACAGGGGGACTTCTCACTCTGATAGAAAG | |
| | (SEQ ID NO: 786) | |
| SRR3989431_2283179_3\|M | CTCGCAATAGGTGATTGCCTGTTTTAAGAATTGAAAC | 37- |
| (SEQ ID NO: 654) | (SEQ ID NO: 719) | 45 |
| | GTTTCAATTCTTAAAACAGGCAATCACCTATTGCGAG | |
| | (SEQ ID NO: 787) | |
| SRR3989431_168870_5\|M | CCCGCAATAGACGATTGCCTATTTTATGGATTGAAAC | 31- |
| (SEQ ID NO: 655) | (SEQ ID NO: 697) | 43 |
| | GTTTCAATCCATAAAATAGGCAATCGTCTATTGCGGG | |
| | (SEQ ID NO: 765) | |
| SRR4754862_508403_7\|P | CTCGCAATAGGTGATTACCTGTTTTAAGGATTGAAAC | 35- |
| (SEQ ID NO: 656) | (SEQ ID NO: 720) | 39 |
| | GTTTCAATCCTTAAAACAGGTAATCACCTATTGCGAG | |
| | (SEQ ID NO: 788) | |
| SRR5468113_274154_3\|M | GTTGCCAAGAGGGTGACTAATTGGGTTAAG | 26- |
| (SEQ ID NO: 657) | (SEQ ID NO: 703) | 38 |
| | CTTAACCCAATTAGTCACCCTCTTGGCAAC | |
| | (SEQ ID NO: 771) | |
| SRR5468149_1084624_1\|P | GTTGCAATGGCTGAAACCCGGTTTACGGGACTGAAAC | 36- |
| (SEQ ID NO: 658) | (SEQ ID NO: 721) | 48 |
| | GTTTCAGTCCCGTAAACCGGGTTTCAGCCATTGCAAC | |
| | (SEQ ID NO: 789) | |
| SRR5574091_1575611_4\|M | CCGACAAACGACTAGAGTTGTCTAGGCAGTTGAAGG | 36- |
| (SEQ ID NO: 659) | (SEQ ID NO: 708) | 43 |
| | CCTTCAACTGCCTAGACAACTCTAGTCGTTTGTCGG | |
| | (SEQ ID NO: 776) | |
| SRR6475630_1852385_2\|M | CTCGCAATAGGTGATTACCTGTTTTAAGGA | 43 |
| (SEQ ID NO: 660) | (SEQ ID NO: 722) | |
| | TCCTTAAAACAGGTAATCACCTATTGCGAG | |
| | (SEQ ID NO: 790) | |
| SRR6475633_3251865_1\|M | CTCGCAATAGGTGATTACCTGTTTTAAGGATTGAAAC | 36- |
| (SEQ ID NO: 661) | (SEQ ID NO: 720) | 48 |
| | GTTTCAATCCTTAAAACAGGTAATCACCTATTGCGAG | |
| | (SEQ ID NO: 788) | |
| 3300031784\|Ga0315899_10084352_2\|M | GCGATCGGGACTGAAAC (SEQ ID NO: 723) | 25- |
| (SEQ ID NO: 662) | GTTTCAGTCCCGATCGC (SEQ ID NO: 791) | 26 |
| 3300031857\|Ga0315909_10035026_2\|M | CGACTAGAGTTGTCTAGGCAGTTGAAGG | 44- |
| (SEQ ID NO: 663) | (SEQ ID NO: 724) | 52 |
| | CCTTCAACTGCCTAGACAACTCTAGTCG | |
| | (SEQ ID NO: 792) | |
| 3300032092\|Ga0315905_10155087_1\|P | CTGACAGGTGACTAGAGTTGTCTAGGCAGTTGAAGG | 33- |
| (SEQ ID NO: 664) | (SEQ ID NO: 725) | 43 |
| | CCTTCAACTGCCTAGACAACTCTAGTCACCTGTCAG | |
| | (SEQ ID NO: 793) | |
| 3300032092\|Ga0315905_10155087_2\|M | CTGACAGGTGACTAGAGTTGTCTAGGCAGTTGAAGG | 33- |
| (SEQ ID NO: 665) | (SEQ ID NO: 725) | 43 |
| | CCTTCAACTGCCTAGACAACTCTAGTCACCTGTCAG | |
| | (SEQ ID NO: 793) | |
| SRR6476078_1283286_3\|P | GTTCCAATTAATCTTAAGTCCTATTAGGGATTGAAAC | 34- |
| (SEQ ID NO: 666) | (SEQ ID NO: 695) | 43 |
| | GTTTCAATCCCTAATAGGACTTAAGATTAATTGGAAC | |
| | (SEQ ID NO: 763) | |
| SRR6476078_1283286_3\|M | GTTCCAATTAATCTTAAGTCCTATTAGGGATTGAAAC | 34- |
| (SEQ ID NO: 667) | (SEQ ID NO: 695) | 43 |
| | GTTTCAATCCCTAATAGGACTTAAGATTAATTGGAAC | |
| | (SEQ ID NO: 763) | |
| SRR7812565_865334_1\|P | CTTGCAACTGGGCTTGGGGACTGAGGATAGTTGAAAC | 35- |
| (SEQ ID NO: 668) | (SEQ ID NO: 683) | 39 |
| | GTTTCAACTATCCTCAGTCCCCAAGCCCAGTTGCAAG | |
| | (SEQ ID NO: 751) | |
| SRR6448207_482583_3\|P | GGCGCAATCGACTTAAGCCCTTAGCAGGGATTGAAAC | 34- |
| (SEQ ID NO: 669) | (SEQ ID NO: 726) | 37 |
| | GTTTCAATCCCTGCTAAGGGCTTAAGTCGATTGCGCC | |
| | (SEQ ID NO: 794) | |
| SRR7244345_343276_44\|P | GTTCAAATAGCCTTTAATCCTTCGTAGGGGTTGAAAG | 33- |
| (SEQ ID NO: 670) | (SEQ ID NO: 727) | 36 |
| | CTTTCAACCCCTACGAAGGATTAAAGGCTATTTGAAC | |
| | (SEQ ID NO: 795) | |
| WP_016456110.1 | CGTCTTGCCCGTGCGCGTGTGGGGTGATC | 32- |
| (SEQ ID NO: 671) | (SEQ ID NO: 728) | 33 |
| | GATCACCCCACACGCGCACGGGCAAGACG | |
| | (SEQ ID NO: 796) | |
| AGZR01000008_18\|M | CGTCTTGCCCGTGCGCGTGTGGGGTGATC | 32- |
| (SEQ ID NO: 672) | (SEQ ID NO: 728) | 33 |
| | GATCACCCCACACGCGCACGGGCAAGACG | |
| | (SEQ ID NO: 796) | |
| SRR8162779_1368970_53\|P | GTTCAAATAGCCTTTAATCCTTCGTAGGGGTTGAAAG | 34- |
| (SEQ ID NO: 673) | (SEQ ID NO: 727) | 37 |
| | CTTTCAACCCCTACGAAGGATTAAAGGCTATTTGAAC | |
| | (SEQ ID NO: 795) | |
| SRR5855413_300079_4\|M | AAAGCCTATCAGGTATTGAAAG | 49- |
| (SEQ ID NO: 674) | (SEQ ID NO: 729) | 51 |
| | CTTTCAATACCTGATAGGCTTT | |
| | (SEQ ID NO: 797) | |
| SRR5855413_1311040_12\|M | GTTGAAATAAGCCTGAATGCCTATCAGGTATTGAAAG | 34- |
| (SEQ ID NO: 675) | (SEQ ID NO: 730) | 48 |
| | CTTTCAATACCTGATAGGCATTCAGGCTTATTTCAAC | |
| | (SEQ ID NO: 798) | |
| SRR5855415_346708_105\|P | GGTTTCTTCAGTTGCCTCGGCTTCAGGTTCAGC | 15 |
| (SEQ ID NO: 676) | (SEQ ID NO: 731) | |
| | GCTGAACCTGAAGCCGAGGCAACTGAAGAAACC | |
| | (SEQ ID NO: 799) | |
| SRR5855420_1331858_6\|7P | GTTGAAATAAGCCTGAATGCCTATCAGGTATTGAAAG | 34- |
| (SEQ ID NO: 677) | (SEQ ID NO: 730) | 44 |
| | CTTTCAATACCTGATAGGCATTCAGGCTTATTTCAAC | |
| | (SEQ ID NO: 798) | |
| SRR5855420_1331858_68\|M | GTTGAAATAAGCCTGAATGCCTATCAGGTATTGAAAG | 34- |
| (SEQ ID NO: 678) | (SEQ ID NO: 730) | 44 |
| | CTTTCAATACCTGATAGGCATTCAGGCTTATTTCAAC | |
| | (SEQ ID NO: 798) | |
| SRR5855435_639752_6\|P | AAAGCCTATCAGGTATTGAAAG | 49- |
| (SEQ ID NO: 679) | (SEQ ID NO: 729) | 51 |
| | CTTTCAATACCTGATAGGCTTT | |
| | (SEQ ID NO: 797) | |
| SRR6201738_878635_2\|M | CTAGCAATAACATTCATCCCCTGGCAGGGATTGAAAG | 34- |
| (SEQ ID NO: 680) | (SEQ ID NO: 732) | 38 |
| | CTTTCAATCCCTGCCAGGGGATGAATGTTATTGCTAG | |
| | (SEQ ID NO: 800) | |
| SRR6201739_286584_1\|P | TCTAGCAATAACATTCATCCCCTGGCAGGGATTGAAA | 31- |
| (SEQ ID NO: 681) | GGGC (SEQ ID NO: 733) | 34 |
| | GCCCTTTCAATCCCTGCCAGGGGATGAATGTTATTGC | |
| | TAGA (SEQ ID NO: 801) | |
| GCL37732.1 (SEQ ID NO: 682) | GTTTCTAGAAGTTCTGAGGACTGATTGTTGTTTAAAC | 35- |
| | (SEQ ID NO: 734) | 41 |
| | GTTTAAACAACAATCAGTCCTCAGAACTTCTAGAAAC | |
| | (SEQ ID NO: 802) | |

TABLE 3111
Non-coding Sequences of Representative
CLUST.057059 Systems

>3300002382|1017448|scaffold9922858_2|M
GCTGGGGAATAGCGGTTATGAAGGTTTATCCTGTCAGATAGCCACAGG
TGGGAACTACTGGAAGAGAGACGAAAATATCAACATCCCAATTAATAC
CAAGAAAGGTATTATTCACGTTAAAGGATACAAATTAGTTAAGGGTGA
TAAACTTTTATTTGACATTCCCCAGCAAGCTGATACTAACGCTGCTAG
AAATCATAAGCAAAATGGTGGTTTTGAACTGGGAGAGGTCAAATATCA
TAATGTTAAGCTAGTTTATCAAAAGCCTAAAAGATTTAAGAAAAAACG
CTTGACAAAGTAATAAGATTAGGCTAATATTAAATTACCGCAGTTTGG
GCGGGCAGCTAATAATATTAGCTTATGACTTCTAAGGCTTTTGCCTAA
AAAGTAAGGGGGAAGGCGACAACCCCCAAAGACCAGCCGGAACTATGG
CTGGCAACTATACCCTCTCTTTTGGCATATCAAAGCTAGGGCAAAAAC
CCCAGACATTCGCCAAAAGCCCAGAACCATGACATTGCAAGAGTTTCG
CCCAGTTTCTTTTAAAGATTCAAGCTGATTTAAGCGGCTGAAATGAGA
TTTTTTAATGTGTAATCGTCTAGCCTGGGAACATATATGCAACTTGCA
ATTAACTCAGTTCCTCAAGTCCGGTTTCACCTATTACCTAATAGATAG
GCACGCCACAGGAGACAATTCCCTTGCAATTAACTCAGTTCCCCAAGT
CCGGTTTCACCAATGACCAGGTGCGCGATCGCACCCCACAAGAGCAAA
CTTGCAATTAA
(SEQ ID NO: 735)
>3300005805|Ga0079957_1019747_2|M
AATTATGTCTCCGTTTGCTACTTGCTTAAACAGTTTAGATATGTTGGG
CTTATCTCCCCAGTAACAGCCAAACTCCAAAGATAGCTTTTCAAGTTC
AGCTTTTTCTTCCTCGGATATAGAGAGCGTGATACTCTCTTGTTTTCT
AGTCATAATGTTATTTTAGCCTAGCCTCTCTTGTCTCTATATCCCTAT
AATATCCTTATAGTTTTCCAGTGCCAACCCTAAAGTATACGGAATTTT
GCAATTGTTTAATTGTTGTATAAATAACTTGCTATGATGCTATACTAA
TAACTCGGTTGTCACCTGTGCAATACCGTCTGCCTAATCAGCAGACAT
TTATTAATCTACTAATTGTCAATAAGTAATAAGAACCCAGTGGTTAAA
ATGACGGGTTCTGTGGCTGTTTTCAAACAAGATTATTTGTCTACATTG
TTTACATTGTCTGACATTATTGTCTTACAAAAGAAAGCAGGAATTGGT
TGAAACATGACTGGGGTAAAGATTTGGGGTGTATGCAACTATCAGTTA
ATGTCTAATTTTTAATATAAAAAATATTTTTAAATTACCAAATTTGTG
CTATCCTAAATAAATACCAAAAAACGCGGTTAGTACCAATAACCACGT
TTCTATTACCAGGTACTTAGCCGCTAGTTTGGGGGAACGGCAACTAAC
AATATAGCACAAAACAGCTATTTACATCAACACCAATTGACAAAAACA
ACAGGAAGACGACGGACATGAAGACAATTAAGATTAAAATCAAACTCT
CATCAGAACAGGCGGCTGTGTGCGATCGCTACTTAGATGAATTAACAT
GGCTGTGGAATTTAGCACTTTCTAACCAACTACACAATCACTGCGTAG
ATTGGTATGCTTGGGCTGCTAAATTATTTGACGTTGCCCAACAAGCTG
ATACCAATGCTGCTAGAAATCATAAACAGAATGGTGGTTTTGAATTGG
GAGAGGTCAAATACCATAATGTTAGGCTGGTTTATCAAAAGCCTAAAA
GATTTAAGAAAAAACGCTTGACAAAGTAATAGGACTAGGTTAATATTA
AATTACCGCAGTTTGGACGGGTAGCTAATGTAATGTTAGCTTATGACT
TGTAAGGGTTTTACCCTAAAAAGTAAGGGGGAAGGCGACGACCCCCAA
AGACCAGCCGGAACTATGGCTGGCAACTATCTCATCTTTTTGGCATAT
CAAAGCTAGGGTAAAAACCCTGGACATTCGCCAAAAGTCCAGAACCAT
GACATTGCAAGAGTTTCGCTCAGTTTTCTTGCGACTGGGCTTAGAGAC
TGAGGATAGTTGAAACTCGCTCAGTTTCTTTTAAAGACCTAAGCTGAT
TTAAGCGGCTGAAATGAGATTTTTTAGCAGGTCCGCCAGAATTGCCTT
TGAAACCCTTGACTTATAAAGCTCTTGAAAGCCGGGAAAACCGGACTT
GAGGAACTGGGTTAATTGCAAGGCAAAGAATCAAAGCAAGACAAAGAA
ACGCTGAAACAGAATTTAATACTTTAACTTACATAACTCCCATCCCAA
ATACATAGCATGGGAGTTTTGCAACCCTGGACAATCTCTCATTATTTC
(SEQ ID NO: 736)
>3300023179|Ga0214923_10000634_60|P
GGCATTTTCTACCTTGCAAGAAAGTTAAAAGCTGTCCCGGTGAAAGGG
ATTGGGTGTAAAGTTTCAGGAATTGATATTATTTTTGCCATTAAAGGG
ATTGATTGTAGTTGTGTGTGGAATCCATCGTTTTTATTAATCCAATAT
CTTTAATGGCTATTTTTATCCAGAGTCAGGTTTTCCAGCTATGGAAAA
TCCACAAGTGGAAAAACCACAAGTGGAAAATCTGCTATATAATAAATA
TCTATTCCAATAAGTATTTAATTAAATAAGTATTTAAGAAAAAAGAAG
TATTTACGCTTTCTCTCATTAAAGAGGATTTAAGAAAAGAACTAACAC
AACAGATATCTGTTATTCTTGACTTAAAGACAAATATTTCTCTCAAGC
AGATAAATCTCTCATAGGATCAGGTTATGCCCGCCCGTTGCGCTTAAA
CACTATTACACAAAAAGCGAATAGATTTTGTTTGAGAATTTATAAAGC
AATTTTAAAGCGTACTTATTGCATAAATAAAAAATACCCACTAACACG
CTTTAGTGGGTATCAGGTTGAAACCTACAATGCAATTGTAATAACTAT
GTTTCCTGGTTTATAAATATAGTCCATCTTATTTTTTGTTTACTGTCA
AGTGGTTTTAAAAAATATTTGTTTTGTTTGTTTGCCTGTATTAATTAA
ATTATTAATAAATCACTTAATATTTATCTACTTATGCTATCATTAATA
AGTACCCAAAAACGTGATTATTCAAAGTAATCACGTTTTTATTACCAG
GTACTTAGTCGCTAGTTTGGATAACGACCTTGACATAATAGCATGAAA
ACAATCAAAATCAAAATCAAACTCACCACTGATCAGGTGCAACTGTGC
GATCGCTATTTGGAAGAATTGACATGGCTGTGGAATTTAACACTTTCT
AATCAACTACATAATCACTGTGTGACGTGGTACGATTGGGCGGCTAAA
TTTGATATTCCCCAGCAAGCTGATACTAACGCTGCTAGAAATCATAAG
CAGAATGGTGGTTTTGAACTGGGAGAGGTCAAGTATCATAATGTTAAG
CTAGTTTATCAAAAGCCTAAAAGATTTAAGAAAAAACGCTTGACAAAC
CAATAGGATTAGGTTAATATTAAATTACCGCAGTTTGGGCGGGTTGCT
AATAATATTAGCTTATGACTTCTAAGGCTTTTGCCTAAAAAGTAAGGG
GGAAGGCGACAACCCCCAAAGACCAGCCGGAACTATGGCTGGCAACTA
TCTCATCTTTTTGGCATATCAAAGCTAGGGCAAAAACCCCAGACATTC
GCCAAAAGCCCAGAACCATGACATTGCAAGAGTTTCGCCCAGTTTCTT
TTAAAGACCCAAGCTGATTTAAGCGGCTGAAATGAGATTTTTTAACAG
GTCCGCCAAAATCGCTTCTGAAACCCTTGATTTATAAAGCCTTTAAAC
CCCGGGGAAACGAAGCGAAATGAAACTTTGAACTAAGGACGGAACATC
TGAATCAAACTCAGTTCCAATTTAAACACTTTCTCACTTGATGGTGTC
CACTTAAACGTTTCGCAACAATACAAAGTGGAATTAAAGTAGAAAGGC
TTACCTAAATTATTGGTTAAAAAAGTATCAATCTCTGA
(SEQ ID NO: 737)
>OGWG01000020_4|M
CCCTGCCTTTGGAATCCTTACTGTGATTGGGTTCTGGAAGCAGTTTGC
GAGGTTCAATTCAAAAAGTCCATTTCAGCCGCTGAGATGTGCTTGTAA
TCTACCTCTAATCCTTACGTGGTAAGGCTGCGAGGTTCGTTTGATGGT
GTGCCATGTTTCCGGCACATCTATGTAGGTTGTCACCTTCCTACTTAC
TCCCGGTTAAGAGAGTCTTTGTACCTGGACACCAGATAATCCCGCCAG
AACTGCGGTACAAAACTATTATAACACAGATTTTGGAGTTGTGTGTAG
TTTGTCAAAAATAGTTAAAATTAATCTGTTATTGGATTAGACACTAGT
TTATTTTTATGTTTTATCCCCCTGCCTTTGGAATCCTTGCTGTGATTG
GGTTCTAAAACCAGTTTGCGAGGTTCAATTCAAAAAGTCCATTTCAGC
CGCTGAGATGTGCTTGTAATCTACCTCTAATCCTTACGTGGTAAGGCT
GCGAGGTTCGTTAGATGGTGTGCCATTATTCCGGCACATCTATGTAGG
TTGTCACCTTCCTACTTACTCCCAGTTAAGAGAGTCTTTGTACCTGGA
CACCAGGTAATTCCGCCAGAACTGCGGTACAAAACTAGTATAGCATAA
ATTTTGGAGTTGTGTTATAATTTTAAAAAATCAGTAGCAGGTGACCAT
AATGAAGACGATTTGTATCAAGGTAAAGTTATCGCCAGAACAGGAGAG
AGAGTTTAGTAATGCCACTCAAGAACTGGAGTGGATATGGAATTTCAG
TTTAAAGACGTATATACATAACCACTGTGTAAAGTGGTATGAATGGGC
TACGGGGGAGCTAGGAGAGGTCAAATACCATAATTTAAGCCTAGAATA
TAATGTTTCCGCACGTTGGAAACATAGAAATAGGAACAAGGTAAAACC
CGTTGTTTCCGTTACTAAGCTGCCTACCACAAGCACTCCCGTTCCTAA
TAAACCTAAATGAACTAAGTTATTGACAATATGAGTTATCAATTTAAA
TCATCTACCGCCAATCCCGTGTTTTTCCGGACTTATAGCCGTTTGACT
CCAGATGGTAGGGAATCATGGCAAGATGTGTGTAAACGCACCATATCT
GGTATGTCTGAACTAGGACGTTTTACACCAGAGGAA
(SEQ ID NO: 738)

### Example 14—Functional Validation of Engineered CLUST.057059 CRISPR-Cas System

Having identified components of CLUST.057059 CRISPR-Cas systems, a locus from the metagenomic source designated 3300023179 (SEQ ID NO: 601) was selected for functional validation.

### DNA Synthesis and Effector Library Cloning

To test the activity of the exemplary CLUST.057059 CRISPR-Cas system, the system was designed and synthesized using a pET28a(+) vector. Briefly, an E. coli codon-optimized nucleic acid sequence encoding the CLUST.057059 3300023179 effector (SEQ ID NO: 601 shown in TABLE 29) was synthesized (Genscript) and cloned into a custom expression system derived from pET-28a(+) (EMD-Millipore). The vector included the nucleic acid encoding CLUST.057059 effectors under the control of a lac promoter and an E. coli ribosome binding sequence. The vector also included an acceptor site for a CRISPR array library driven by a J23119 promoter following the open reading frame for the CLUST.057059 effector. The non-coding sequence used for the CLUST.057059 3300023179 effector (SEQ ID NO: 601) is set forth in SEQ ID NO: 619, as shown in TABLE 31. A separate condition was tested, wherein the CLUST.057059 3300023179 effector (SEQ ID NO: 601) was individually cloned into pET28a(+) without the non-coding sequence. See FIG. 1A.

An oligonucleotide library synthesis (OLS) pool containing “repeat-spacer-repeat” sequences was computationally designed, where “repeat” represents the consensus direct repeat sequence found in the CRISPR array associated with the effector, and “spacer” represents sequences tiling the pACYC184 plasmid or E. coli essential genes. In particular, the repeat sequence used for the CLUST.057059 3300023179 effector (SEQ ID NO: 601) is set forth in SEQ ID NO: 611, as shown in TABLE 30. The spacer length was determined by the mode of the spacer lengths found in the endogenous CRISPR array. The repeat-spacer-repeat sequence was appended with restriction sites enabling the bi-directional cloning of the fragment into the aforementioned CRISPR array library acceptor site, as well as unique PCR priming sites to enable specific amplification of a specific repeat-spacer-repeat library from a larger pool.

Next, the repeat-spacer-repeat library was cloned into the plasmid using the Golden Gate assembly method. Briefly, each repeat-spacer-repeat was first amplified from the OLS pool (Agilent Genomics) using unique PCR primers and pre-linearized the plasmid backbone using BsaI to reduce potential background. Both DNA fragments were purified with Ampure XP (Beckman Coulter) prior to addition to Golden Gate Assembly Master Mix (New England Biolabs) and incubated per the manufacturer's instructions. The Golden Gate reaction was further purified and concentrated to enable maximum transformation efficiency in the subsequent steps of the bacterial screen.

The plasmid library containing the distinct repeat-spacer-repeat elements and CRISPR effectors was electroporated into E. Cloni electrocompetent E. coli (Lucigen) using a Gene Pulser Xcell® (Bio-rad) following the protocol recommended by Lucigen. The library was either co-transformed with purified pACYC184 plasmid or directly transformed into pACYC184-containing E. Cloni electrocompetent E. coli (Lucigen), plated onto agar containing chloramphenicol (Fisher), tetracycline (Alfa Aesar), and kanamycin (Alfa Aesar) in BioAssay® dishes (Thermo Fisher), and incubated for 10-12 hours at 37° C. After estimation of approximate colony count to ensure sufficient library representation on the bacterial plate, the bacteria were harvested, and plasmid DNA WAS extracted using a QIAprep Spin Miniprep® Kit (Qiagen) to create an “output library.” By performing a PCR using custom primers containing barcodes and sites compatible with Illumina sequencing chemistry, a barcoded next generation sequencing library was generated from both the pre-transformation “input library” and the post-harvest “output library,” which were then pooled and loaded onto a Nextseq 550 (Illumina) to evaluate the effectors. At least two independent biological replicates were performed for each screen to ensure consistency. See FIG. 1B.

### Bacterial Screen Sequencing Analysis

Next generation sequencing data for screen input and output libraries were demultiplexed using Illumina bcl2fastq. Reads in resulting fastq files for each sample contained the CRISPR array elements for the screening plasmid library. The direct repeat sequence of the CRISPR array was used to determine the array orientation, and the spacer sequence was mapped to the source (pACYC184 or E. Cloni) or negative control sequence (GFP) to determine the corresponding target. For each sample, the total number of reads for each unique array element (rₐ) in a given plasmid library was counted and normalized as follows: (rₐ+1)/total reads for all library array elements. The depletion score was calculated by dividing normalized output reads for a given array element by normalized input reads.

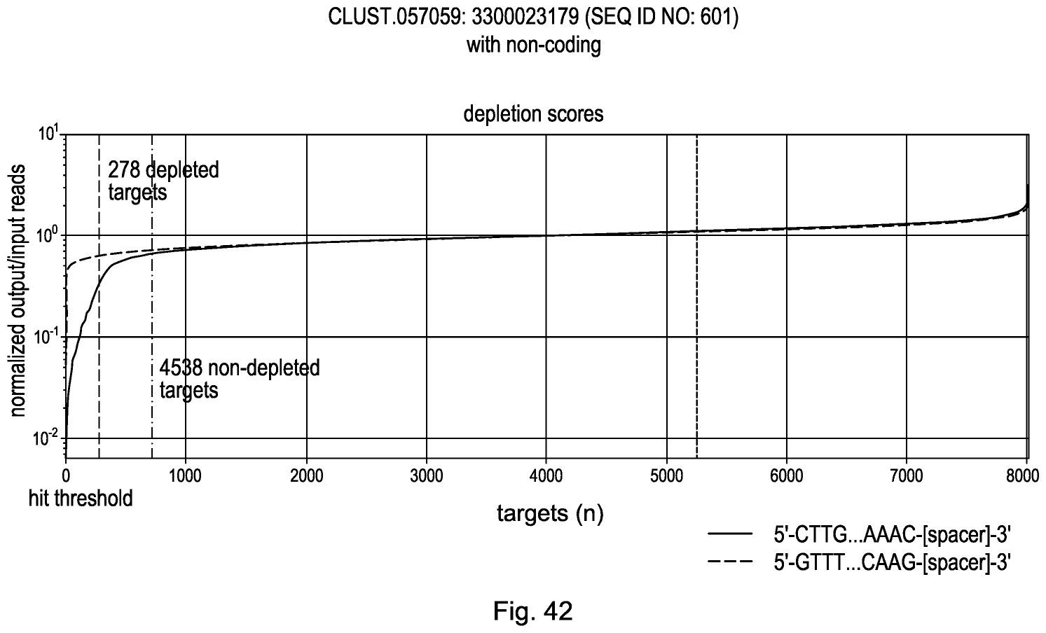

To identify specific parameters resulting in enzymatic activity and bacterial cell death, next generation sequencing (NGS) was used to quantify and compare the representation of individual CRISPR arrays (i.e., repeat-spacer-repeat) in the PCR product of the input and output plasmid libraries. The array depletion ratio was defined as the normalized output read count divided by the normalized input read count. An array was considered to be “strongly depleted” if the depletion ratio was less than 0.3 (more than 3-fold depletion), depicted by the blue dashed line in FIG. 42. When calculating the array depletion ratio across biological replicates, the maximum depletion ratio value for a given CRISPR array was taken across all experiments (i.e. a strongly depleted array must be strongly depleted in all biological replicates). A matrix including array depletion ratios and the following features were generated for each spacer target: target strand, transcript targeting, ORI targeting, target sequence motifs, flanking sequence motifs, and target secondary structure. The degree to which different features in this matrix explained target depletion for CLUST.057059 systems was investigated.

FIG. 42 shows the degree of interference activity of the engineered compositions, with a non-coding sequence, by plotting for a given target the normalized ratio of sequencing reads in the screen output versus the screen input. The results are plotted for each DR transcriptional orientation. In the functional screen for each composition, an active effector complexed with an active RNA guide will interfere with the ability of the pACYC184 to confer E. coli resistance to chloramphenicol and tetracycline, resulting in cell death and depletion of the spacer element within the pool. Comparison of the results of deep sequencing the initial DNA library (screen input) versus the surviving transformed E. coli (screen output) suggests specific target sequences and DR transcriptional orientations that enable an active, programmable CRISPR system. The screen also indicates that the effector complex is only active with one orientation of the DR. As such, the screen indicated that the CLUST.057059 3300023179 effector was active in the “forward” orientation (5′-CTTG . . . AAAC-[spacer]-3′) of the DR (FIG. 42). The CLUST.057059 3300023179 effector did not retain activity in the absence of the non-coding sequence, indicating that CLUST.057059 effectors require a tracrRNA.

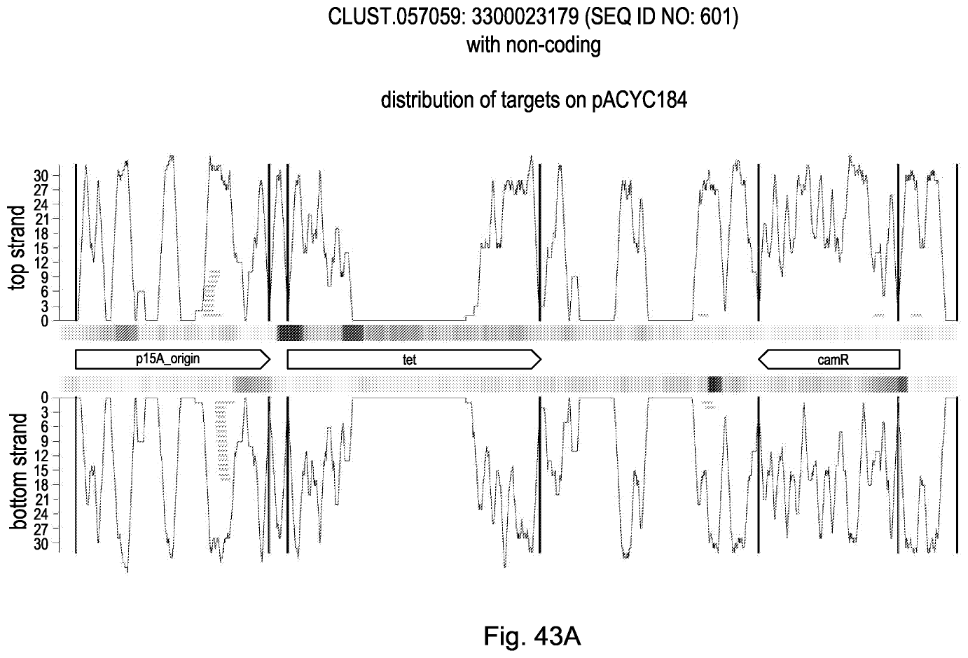

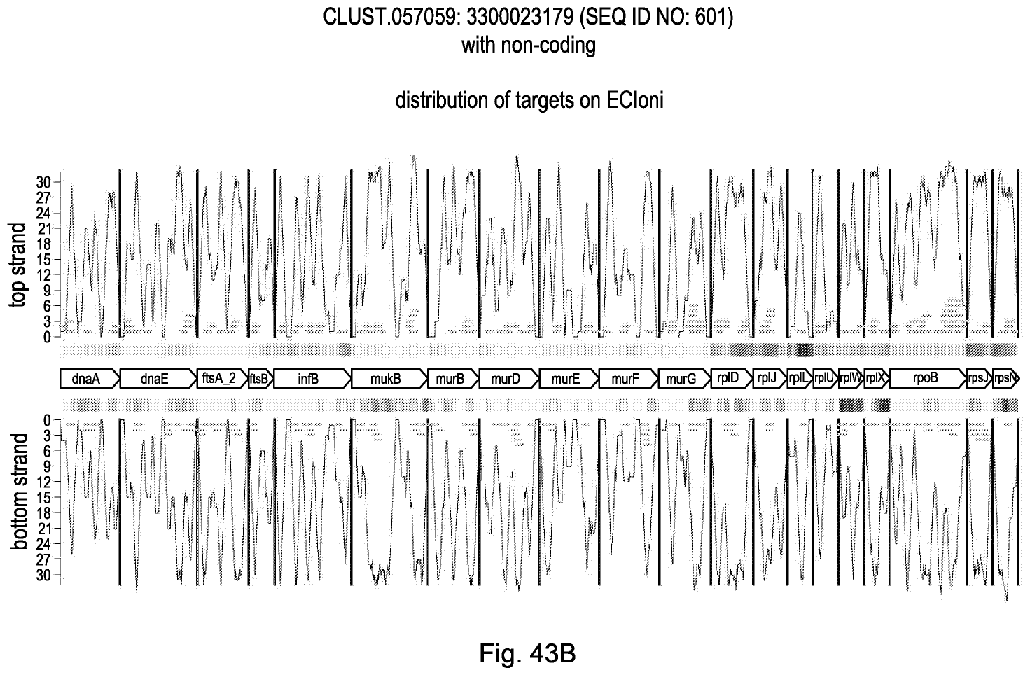

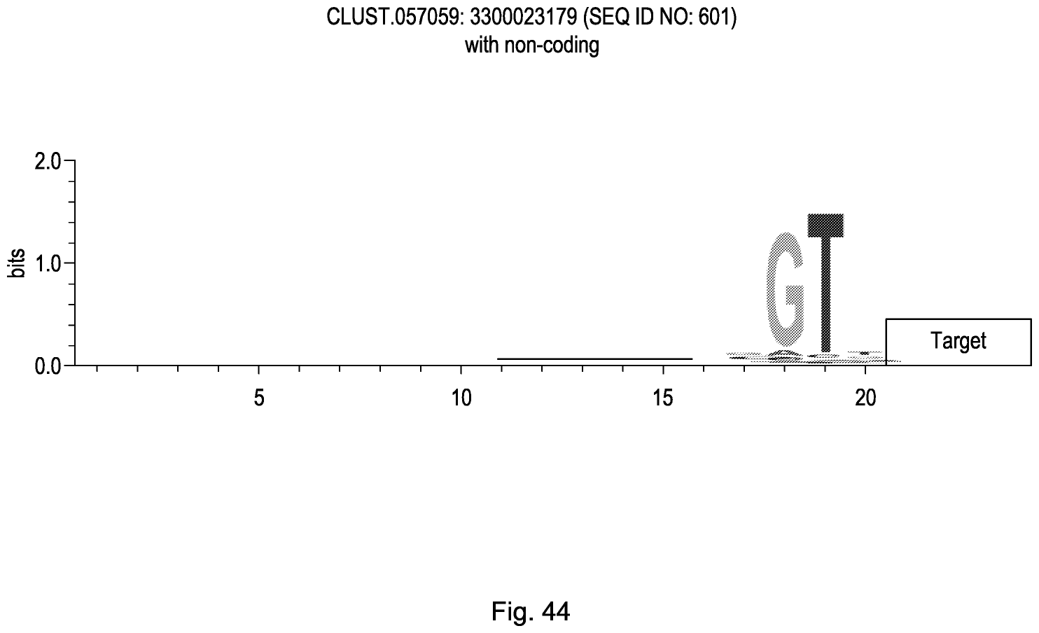

FIG. 43A and FIG. 43B depict the location of strongly depleted targets for the CLUST.057059 3300023179 effector (plus non-coding sequence) targeting pACYC184 and E. coli E. Cloni essential genes, respectively. Flanking sequences of depleted targets were analyzed to determine the PAM sequence for CLUST.057059 3300023179. A WebLogo representation (Crooks et al., Genome Research 14: 1188-90, 2004) of the PAM sequence for CLUST.057059 3300023179 is shown in FIG. 44, where the “20” position corresponds to the nucleotide adjacent to the 5′ end of the target.

### Example 15—Targeting of Mammalian Genes

This Example describes an indel assessment on a mammalian target by the effector disclosed herein introduced into mammalian cells by transient transfection.

An effector described herein is cloned into a pcda3.1 backbone (Invitrogen). The plasmid is then maxi-prepped and diluted to 1 μg/L. For RNA guide preparation, a dsDNA fragment encoding an RNA guide is derived by ultramers containing the target sequence scaffold, and the U6 promoter. Ultramers are resuspended in 10 mM Tris-HCl at a pH of 7.5 to a final stock concentration of 100 μM. Working stocks are subsequently diluted to 10 μM, again using 10 mM Tris.HCl to serve as the template for the PCR reaction. The amplification of the RNA guide is done in 50 μL reactions with the following components: 0.02 μl of aforementioned template, 2.5 μl forward primer, 2.5 μl reverse primer, 25 μL NEB HiFi Polymerase, and 20 μl water. Cycling conditions are: 1×(30 s at 98° C.), 30×(10 s at 98° C., 15 s at 67° C.), 1×(2 min at 72° C.). PCR products are cleaned up with a 1.8×SPRI treatment and normalized to 25 ng/μL.

The sequence of a target locus is selected as described herein. For example, a target locus adjacent to a PAM sequence of TABLE 34 is selected.

TABLE 34
PAM sequence for target selection.

| Effector Family | PAM Sequence |
|---|---|
| CLUST.133120 | 5′-TTN-3′ |
| | 5′-TN-3′ |
| CLUST.099129 | 5′-GTN-3′ |
| | 5′-TG-3′ |
| | 5′-TR-3′ |
| | 5′-RATG-3′ (SEQ ID NO: 920) |
| CLUST.342201 | 5′-AAG-3′ |
| | 5′-AAD-3′ |
| | 5′-AAR-3′ |
| | 5′-RAAG-3′ (SEQ ID NO: 921) |
| | 5′-RAAR-3′ (SEQ ID NO: 922) |
| | 5′-RAAD-3′ (SEQ ID NO: 923) |
| CLUST.195009 | 5′-TTN-3′ |
| CLUST.057059 | 5′-GTN-3′ |

A crRNA sequence is selected as described herein. For example, a crRNA comprises a direct repeat sequence of the length and sequence described herein. Non-limiting examples of direct repeats are shown in TABLE 35.

TABLE 35
Direct Repeat for crRNA design.

| Effector | Direct Repeat Sequence |
|---|---|
| CLUST.13312 | CCAACCAATGCCAGCGCGACGGCTTATGAGTCG |
| 0 3300027740 | CGAC (SEQ ID NO: 51) |
| (SEQ ID | AATGCCAGCGCGACGGCTTATGAGTCGCGAC |
| NO: 1) | (SEQ ID NO: 85) |
| CLUST.099129 | AGTCGCGAATAACTGTTCAGCGGAGAAGCCGCT |
| SRR6837557 | GAAAC (SEQ ID NO: 146) |
| (SEQ ID | ATAACTGTTCAGCGGAGAAGCCGCTG |
| NO: 101) | (SEQ ID NO: 181) |
| CLUST.34220 | GTTCACCCCACGGGTGCGTGGAGTGATGG |
| 1 3300006417 | (SEQ ID NO: 342) |
| (SEQ ID | |
| NO: 301) | |
| CLUST.195009 | CCAGCAACAGCCGCGTGGGGCTACTAGTACTGC |
| SRR6201554 | GAC (SEQ ID NO: 522) |
| (SEQ ID | CAACAGCCGCGTGGGGCTACTAGTACTGCG |
| NO: 501) | (SEQ ID NO: 535) |
| CLUST.05705 | CTTGCAACTGGGCTTGGGGACTGAGGATAGTTG |
| 9 3300023179AAA | C (SEQ ID NO: 683) |
| (SEQ ID | |
| NO: 601) | |

Approximately 16 hours prior to transfection, 100 μl of 25,000 HEK293T cells in DMEM/10% FBS+Pen/Strep are plated into each well of a 96-well plate. On the day of transfection, the cells are 70-90% confluent. For each well to be transfected, a mixture of 0.5 μl of Lipofectamine 2000 and 9.5 μl of Opti-MEM is prepared and then incubated at room temperature for 5-20 minutes (Solution 1). After incubation, the lipofectamine:OptiMEM mixture is added to a separate mixture containing 182 ng of effector plasmid and 14 ng of crRNA and water up to 10 μL (Solution 2). In the case of negative controls, the crRNA is not included in Solution 2. The solution 1 and solution 2 mixtures are mixed by pipetting up and down and then incubated at room temperature for 25 minutes. Following incubation, 20 μL of the Solution 1 and Solution 2 mixture are added dropwise to each well of a 96 well plate containing the cells. 72 hours post transfection, cells are trypsinized by adding 10 μL of TrypLE to the center of each well and incubated for approximately 5 minutes. 100 μL of D10 media is then added to each well and mixed to resuspend cells. The cells are then spun down at 500 g for 10 minutes, and the supernatant is discarded. QuickExtract buffer is added to ⅕ the amount of the original cell suspension volume. Cells are incubated at 65° C. for 15 minutes, 68° C. for 15 minutes, and 98° C. for 10 minutes.

Samples for Next Generation Sequencing are prepared by two rounds of PCR. The first round (PCR1) is used to amplify specific genomic regions depending on the target. PCR1 products are purified by column purification. Round 2 PCR (PCR2) is done to add Illumina adapters and indexes. Reactions are then pooled and purified by column purification. Sequencing runs are done with a 150 cycle NextSeq v2.5 mid or high output kit.

Percentages of indels in the target locus in HEK293T cells following transfection are calculated. Indel percentages over background are indicative of nuclease activity in mammalian cells.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An engineered, non-naturally occurring Clustered Regularly Interspaced Short Palindromic Repeat (CRISPR)—Cas system of CLUST.133120, CLUST.099129, CLUST.342201, CLUST.195009, or CLUST.057059 comprising:
(a) a CRISPR-associated protein or a nucleic acid encoding the CRISPR-associated protein, wherein the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 1-50, 101-145, 301-341, 501-521, or 601-682; and
(b) an RNA guide comprising a direct repeat sequence and a spacer sequence capable of hybridizing to a target nucleic acid;
wherein the CRISPR-associated protein is capable of binding to the RNA guide and of modifying the target nucleic acid sequence complementary to the spacer sequence.

2. The system of claim 1, wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 51-72, 85-87, 95-100, or 900-915.

3. The system of claim 1 or 2, wherein the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 1 or SEQ ID NO: 2.

4. The system of claim 3, wherein the CRISPR-associated protein is capable of recognizing a protospacer adjacent motif (PAM) sequence, wherein the PAM sequence comprises a nucleic acid sequence set forth as 5′-TTN-3′ or 5′-TN-3′.

5. The system of any of the preceding claims, wherein the spacer sequence of the RNA guide comprises between about 15 nucleotides to about 55 nucleotides.

6. The system of any of the preceding claims, wherein the spacer sequence of the RNA guide comprises between 20 and 35 nucleotides.

7. The system of claim 1 or 2, wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 146-162.

8. The system of any of claims 1, 2, or 7, wherein the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 101, SEQ ID NO: 102, or SEQ ID NO: 103.

9. The system of any of claims 1, 2, 7, or 8, wherein the CRISPR-associated protein is capable of recognizing a protospacer adjacent motif (PAM) sequence, wherein the PAM sequence comprises a nucleic acid sequence set forth as 5′-GTN-3′, 5′-TG-3′, 5′-TR-3′, or 5′-RATG-3′.

10. The system of any of claims 1, 2, or 7-9, wherein the spacer sequence of the RNA guide comprises between about 15 nucleotides to about 55 nucleotides.

11. The system of any of claims 1, 2, or 7-10, wherein the spacer sequence of the RNA guide comprises between 26 and 51 nucleotides.

12. The system of claim 1 or 2, wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 342-362.

13. The system of any of claims 1, 2, or 12, wherein the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 301.

14. The system of any of claims 1, 2, 12, or 13, wherein the CRISPR-associated protein is capable of recognizing a protospacer adjacent motif (PAM) sequence, wherein the PAM sequence comprises a nucleic acid sequence set forth as 5′-AAG-3′, 5′-AAD-3′, 5′-AAR-3′, 5′-RAAG-3′ (SEQ ID NO: 921), 5′-RAAR-3′ (SEQ ID NO: 922), 5′-RAAD-3′ (SEQ ID NO: 923).

15. The system of any of claims 1, 2, or 12-14, wherein the spacer sequence of the RNA guide comprises between about 12 nucleotides to about 62 nucleotides.

16. The system of claim 15, wherein the spacer sequence of the RNA guide comprises between 19 and 40 nucleotides.

17. The system of claim 1 or 2, wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 522-532.

18. The system of claim 1 or 2, wherein the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 501.

19. The system of any one of claims 1, 2, or 18, wherein the CRISPR-associated protein is capable of recognizing a protospacer adjacent motif (PAM) sequence, wherein the PAM sequence comprises a nucleic acid sequence set forth as 5′-TTN-3′.

20. The system of any one of claims 1, 2, 18, or 19, wherein the spacer sequence of the RNA guide comprises between about 15 nucleotides to about 55 nucleotides.

21. The system of claim 20, wherein the spacer sequence of the RNA guide comprises between 20 and 39 nucleotides.

22. The system of claim 1 or 2, wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 683-734.

23. The system of any one of claims 1, 2, or 22, wherein the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 601.

24. The system of any one of claims 1, 2, 22, or 23, wherein the CRISPR-associated protein is capable of recognizing a protospacer adjacent motif (PAM) sequence, wherein the PAM sequence comprises a nucleic acid sequence set forth as 5′-GTN-3′.

25. The system of any one of claims 1, 2, or 22-24, wherein the spacer sequence of the RNA guide comprises between about 15 nucleotides to about 50 nucleotides.

26. The system of claim 25, wherein the spacer sequence of the RNA guide comprises between 20 and 44 nucleotides.

27. The system of any of the preceding claims, wherein the CRISPR-associated protein comprises at least one RuvC domain or at least one split RuvC domain.

28. The system of any of the preceding claims, wherein the CRISPR-associated protein comprises a catalytic residue (e.g., aspartic acid or glutamic acid).

29. The system of any of the preceding claims, wherein the CRISPR-associated protein cleaves the target nucleic acid.

30. The system of any of the preceding claims, wherein the CRISPR-associated protein further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

31. The system of any of the preceding claims, wherein the nucleic acid encoding the CRISPR-associated protein is codon-optimized for expression in a cell.

32. The system of any of the preceding claims, wherein the nucleic acid encoding the CRISPR-associated protein is operably linked to a promoter.

33. The system of any of the preceding claims, wherein the nucleic acid encoding the CRISPR-associated protein is in a vector.

34. The system of claim 33, wherein the vector comprises a retroviral vector, a lentiviral vector, a phage vector, an adenoviral vector, an adeno-associated vector, or a herpes simplex vector.

35. The system of any of the preceding claims, wherein the target nucleic acid is a DNA molecule.

36. The system of any of the preceding claims, wherein the target nucleic acid comprises a PAM sequence.

37. The system of any of the preceding claims, wherein the CRISPR-associated protein comprises non-specific nuclease activity.

38. The system of any of the preceding claims, wherein recognition of the target nucleic acid by the CRISPR-associated protein and RNA guide results in a modification of the target nucleic acid.

39. The system of claim 38, wherein the modification of the target nucleic acid is a double-stranded cleavage event.

40. The system of claim 38, wherein the modification of the target nucleic acid is a single-stranded cleavage event.

41. The system of claim 38, wherein the modification of the target nucleic acid results in an insertion event.

42. The system of claim 38, wherein the modification of the target nucleic acid results in a deletion event.

43. The system of any one of claims 38-42, wherein the modification of the target nucleic acid results in cell toxicity or cell death.

44. The system of any of the preceding claims, further comprising a donor template nucleic acid.

45. The system of claim 44, wherein the donor template nucleic acid is a DNA molecule.

46. The system of claim 44, wherein the donor template nucleic acid is an RNA molecule.

47. The system of any of the preceding claims, wherein the system does not comprise a tracrRNA.

48. The system of any of the preceding claims, wherein the CRISPR-associated protein is self-processing.

49. The system of any of the preceding claims, wherein the system is present in a delivery composition comprising a nanoparticle, a liposome, an exosome, a microvesicle, or a gene-gun.

50. The system of any of the preceding claims, within a cell.

51. The system of claim 50, wherein the cell is a eukaryotic cell, e.g., a mammalian cell, e.g., a human cell.

52. The system of claim 50, wherein the cell is a prokaryotic cell.

53. A cell comprising:
(a) a CRISPR-associated protein or a nucleic acid encoding the CRISPR-associated protein, wherein the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 1-50; and
(b) an RNA guide comprising a direct repeat sequence and a spacer sequence capable of hybridizing to a target nucleic acid.

54. The cell of claim 53, wherein the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 1 or SEQ ID NO: 2.

55. The cell of claim 53 or 54, wherein the CRISPR-associated protein is capable of recognizing a PAM sequence comprising a nucleic acid sequence set forth as 5′-TTN-3′ or 5′-TN-3′.

56. The cell of any of claims 53-55, wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 51-72, 85-87, 95-100, or 900-915.

57. The cell of any of claims 53-56, wherein the spacer sequence comprises between about 15 nucleotides to about 55 nucleotides.

58. The cell of any of claims 53-57, wherein the spacer sequence comprises between 20 and 35 nucleotides.

59. A cell comprising:
(a) a CRISPR-associated protein, wherein the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 101-145; and
(b) an RNA guide comprising a direct repeat sequence and a spacer sequence capable of hybridizing to a target nucleic acid.

60. The cell of claim 59, wherein the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 101, SEQ ID NO: 102, or SEQ ID NO: 103.

61. The cell of claim 59 or 60, wherein the CRISPR-associated protein is capable of recognizing a PAM sequence comprising a nucleic acid sequence set forth as 5′-GTN-3′, 5′-TG-3′, 5′-TR-3′, or 5′-RATG-3′.

62. The cell of any of claims 59-61, wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 146-162.

63. The cell of any of claims 59-62, wherein the spacer sequence comprises between about 15 nucleotides to about 55 nucleotides.

64. The cell of any of claims 59-63, wherein the spacer sequence comprises between 26 and 51 nucleotides.

65. A cell comprising:
(a) a CRISPR-associated protein, wherein the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 301-341; and
(b) an RNA guide comprising a direct repeat sequence and a spacer sequence capable of hybridizing to a target nucleic acid.

66. The cell of claim 65, wherein the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 301.

67. The cell of claim 65 or 66, wherein the CRISPR-associated protein is capable of recognizing a PAM sequence comprising a nucleic acid sequence set forth as 5′-AAG-3′, 5′-AAD-3′, 5′-AAR-3′, 5′-RAAG-3′ (SEQ ID NO: 921), 5′-RAAR-3′ (SEQ ID NO: 922), 5′-RAAD-3′ (SEQ ID NO: 923).

68. The cell of any of claims 65-67, wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 342-362.

69. The cell of any of claims 65-68, wherein the spacer sequence comprises between about 12 nucleotides to about 62 nucleotides.

70. The cell of any of claims 65-69, wherein the spacer sequence comprises between 19 and 40 nucleotides.

71. A cell comprising:
(a) a CRISPR-associated protein, wherein the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 501-521; and
(b) an RNA guide comprising a direct repeat sequence and a spacer sequence capable of hybridizing to a target nucleic acid.

72. The cell of claim 71, wherein the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 501.

73. The cell of claim 71 or 72, wherein the CRISPR-associated protein is capable of recognizing a PAM sequence comprising a nucleic acid sequence set forth as 5′-TTN-3′.

74. The cell of any of claims 71-73, wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 522-532.

75. The cell of any of claims 71-74, wherein the spacer sequence comprises between about 15 nucleotides to about 55 nucleotides.

76. The cell of any of claims 71-75, wherein the spacer sequence comprises between 20 and 39 nucleotides.

77. A cell comprising:
(a) a CRISPR-associated protein, wherein the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 601-682; and
(b) an RNA guide comprising a direct repeat sequence and a spacer sequence capable of hybridizing to a target nucleic acid.

78. The cell of claim 77, wherein the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 601.

79. The cell of claim 77 or 78, wherein the CRISPR-associated protein is capable of recognizing a PAM sequence comprising a nucleic acid sequence set forth as 5′-GTN-3′.

80. The cell of any of claims 77-79, wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 683-734.

81. The cell of any of claims 77-80, wherein the spacer sequence comprises between about 15 nucleotides to about 50 nucleotides.

82. The cell of any of claims 77-81, wherein the spacer sequence comprises between 20 and 44 nucleotides.

83. The cell of any one of claims 53-82, wherein the cell does not comprise a tracrRNA.

84. The cell of any one of claims 53-83, wherein the cell is a eukaryotic cell, e.g., a mammalian cell, e.g., a human cell.

85. The cell of any one of claims 53-83, wherein the cell is a prokaryotic cell.

86. A method of modifying a target nucleic acid, the method comprising delivering to the target nucleic acid an engineered, non-naturally occurring CRISPR-Cas system comprising:
(a) a CRISPR-associated protein or a nucleic acid encoding the CRISPR-associated protein, wherein the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 1-50; and
(b) an RNA guide comprising a direct repeat sequence and a spacer sequence capable of hybridizing to the target nucleic acid;
wherein the CRISPR-associated protein is capable of binding to the RNA guide; and
wherein recognition of the target nucleic acid by the CRISPR-associated protein and RNA guide results in a modification of the target nucleic acid.

87. The method of claim 86, wherein the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 1 or SEQ ID NO: 2.

88. The method of claim 86 or 87, wherein the CRISPR-associated protein is capable of recognizing a PAM sequence comprising a nucleic acid sequence set forth as 5′-TTN-3′ or 5′-TN-3′.

89. The method of any of claims 86-88, wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 51-72, 85-87, 95-100, or 900-915.

90. The method of any of claims 86-89, wherein the spacer sequence comprises between about 15 nucleotides to about 55 nucleotides.

91. The method of any of claims 86-90, wherein the spacer sequence comprises between 20 and 35 nucleotides.

92. A method of modifying a target nucleic acid, the method comprising delivering to the target nucleic acid an engineered, non-naturally occurring CRISPR-Cas system comprising:
(a) a CRISPR-associated protein, wherein the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 101-145; and
(b) an RNA guide comprising a direct repeat sequence and a spacer sequence capable of hybridizing to the target nucleic acid;
wherein the CRISPR-associated protein is capable of binding to the RNA guide; and
wherein recognition of the target nucleic acid by the CRISPR-associated protein and RNA guide results in a modification of the target nucleic acid.

93. The method of claim 92, wherein the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 101, SEQ ID NO: 102, or SEQ ID NO: 103.

94. The method of claim 92 or 93, wherein the CRISPR-associated protein is capable of recognizing a PAM sequence comprising a nucleic acid sequence set forth as 5′-GTN-3′, 5′-TG-3′, 5′-TR-3′, or 5′-RATG-3′.

95. The method of any of claims 92-94, wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 146-162.

96. The method of any of claims 92-95, wherein the spacer sequence comprises between about 15 nucleotides to about 55 nucleotides.

97. The method of any of claims 92-96, wherein the spacer sequence comprises between 26 and 51 nucleotides.

98. A method of modifying a target nucleic acid, the method comprising delivering to the target nucleic acid an engineered, non-naturally occurring CRISPR-Cas system comprising:
(a) a CRISPR-associated protein, wherein the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 301-341; and
(b) an RNA guide comprising a direct repeat sequence and a spacer sequence capable of hybridizing to the target nucleic acid;
wherein the CRISPR-associated protein is capable of binding to the RNA guide; and
wherein recognition of the target nucleic acid by the CRISPR-associated protein and RNA guide results in a modification of the target nucleic acid.

99. The method of claim 98, wherein the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 301.

100. The method of claim 98 or 99, wherein the CRISPR-associated protein is capable of recognizing a PAM sequence comprising a nucleic acid sequence set forth as 5′-AAG-3′, 5′-AAD-3′, 5′-AAR-3′, 5′-RAAG-3′ (SEQ ID NO: 921), 5′-RAAR-3′ (SEQ ID NO: 922), 5′-RAAD-3′ (SEQ ID NO: 923).

101. The method of any of claims 98-100, wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 342-362.

102. The method of any of claims 98-101, wherein the spacer sequence comprises between about 12 nucleotides to about 62 nucleotides.

103. The method of any of claims 98-102, wherein the spacer sequence comprises between 19 and 40 nucleotides.

104. A method of modifying a target nucleic acid, the method comprising delivering to the target nucleic acid an engineered, non-naturally occurring CRISPR-Cas system comprising:
(a) a CRISPR-associated protein, wherein the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 501-521; and
(b) an RNA guide comprising a direct repeat sequence and a spacer sequence capable of hybridizing to the target nucleic acid;
wherein the CRISPR-associated protein is capable of binding to the RNA guide; and
wherein recognition of the target nucleic acid by the CRISPR-associated protein and RNA guide results in a modification of the target nucleic acid.

105. The method of claim 104, wherein the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 501.

106. The method of claim 104 or 105, wherein the CRISPR-associated protein is capable of recognizing a PAM sequence comprising a nucleic acid sequence set forth as 5′-TTN-3′.

107. The method of any of claims 104-106, wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 522-532.

108. The method of any of claims 104-107, wherein the spacer sequence comprises between about 15 nucleotides to about 55 nucleotides.

109. The method of any of claims 104-108, wherein the spacer sequence comprises between 20 and 39 nucleotides.

110. A method of modifying a target nucleic acid, the method comprising delivering to the target nucleic acid an engineered, non-naturally occurring CRISPR-Cas system comprising:
(a) a CRISPR-associated protein, wherein the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 601-682; and
(b) an RNA guide comprising a direct repeat sequence and a spacer sequence capable of hybridizing to the target nucleic acid;
wherein the CRISPR-associated protein is capable of binding to the RNA guide; and
wherein recognition of the target nucleic acid by the CRISPR-associated protein and RNA guide results in a modification of the target nucleic acid.

111. The method of claim 110, wherein the CRISPR-associated protein is a protein having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in SEQ ID NO: 601.

112. The method of claim 110 or 111, wherein the CRISPR-associated protein is capable of recognizing a PAM sequence comprising a nucleic acid sequence set forth as 5′-GTN-3′.

113. The method of any of claims 110-112, wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence set forth in any one of SEQ ID NOs: 683-734.

114. The method of any of claims 110-113, wherein the spacer sequence comprises between about 15 nucleotides to about 50 nucleotides.

115. The method of any of claims 110-114, wherein the spacer sequence comprises between 20 and 44 nucleotides.

116. A method of binding the system of any one of claims 1-49 to a target nucleic acid in a cell comprising:
(a) providing the system; and
(b) delivering the system to the cell,
wherein the cell comprises the target nucleic acid, wherein the CRISPR-associated protein binds to the RNA guide, and wherein the spacer sequence binds to the target nucleic acid.

117. The method of claim 116, wherein the cell is a eukaryotic cell, e.g., a mammalian cell, e.g., a human cell.

118. The method of any one of claims 86-117, wherein the system does not comprise a tracrRNA.

119. The method of any one of claims 86-118, wherein the target nucleic acid is a DNA molecule.

120. The method of any one of claims 88-119, wherein the target nucleic acid comprises a PAM sequence.

121. The method of any one of claims 88-120, wherein the CRISPR-associated protein comprises non-specific nuclease activity.

122. The method of any one of claims 88-121, wherein the modification of the target nucleic acid is a double-stranded cleavage event.

123. The method of any one of claims 88-121, wherein the modification of the target nucleic acid is a single-stranded cleavage event.

124. The method of any one of claims 88-121, wherein the modification of the target nucleic acid results in an insertion event.

125. The method of any one of claims 88-121, wherein the modification of the target nucleic acid results in a deletion event.

126. The method of any one of claims 122-125, wherein the modification of the target nucleic acid results in cell toxicity or cell death.

127. A method of editing a target nucleic acid, the method comprising contacting the target nucleic acid with the system of any one of claims 1-49.

128. A method of modifying expression of a target nucleic acid, the method comprising contacting the target nucleic acid with a system of any one of claims 1-49.

129. A method of targeting the insertion of a payload nucleic acid at a site of a target nucleic acid, the method comprising contacting the target nucleic acid with a system of any one of claims 1-49.

130. A method of targeting the excision of a payload nucleic acid from a site at a target nucleic acid, the method comprising contacting the target nucleic acid with a system of any one of claims 1-49.

131. A method of non-specifically degrading single-stranded DNA upon recognition of a DNA target nucleic acid, the method comprising contacting the target nucleic acid with a system of any one of claims 1-49.

132. A method of detecting a target nucleic acid in a sample, the method comprising:
(a) contacting the sample with the system of any one of claims 1-49 and a labeled reporter nucleic acid, wherein hybridization of the spacer sequence to the target nucleic acid causes cleavage of the labeled reporter nucleic acid; and
(b) measuring a detectable signal produced by cleavage of the labeled reporter nucleic acid, thereby detecting the presence of the target nucleic acid in the sample.

133. Use of the system of any one of claims 1-49 in an in vitro or ex vivo method of:
(a) targeting and editing a target nucleic acid;
(b) non-specifically degrading a single-stranded nucleic acid upon recognition of the nucleic acid;
(c) targeting and nicking a non-spacer complementary strand of a double-stranded target upon recognition of a spacer complementary strand of the double-stranded target;
(d) targeting and cleaving a double-stranded target nucleic acid;
(e) detecting a target nucleic acid in a sample;
(f) specifically editing a double-stranded nucleic acid;
(g) base editing a double-stranded nucleic acid;
(h) inducing genotype-specific or transcriptional-state-specific cell death or dormancy in a cell;
(i) creating an indel in a double-stranded nucleic acid target;
(j) inserting a sequence into a double-stranded nucleic acid target; or
(k) deleting or inverting a sequence in a double-stranded nucleic acid target.

134. A method of introducing an insertion or deletion into a target nucleic acid in a mammalian cell, comprising a transfection of:
(a) a nucleic acid sequence encoding a CRISPR-associated protein, wherein the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 1-50, 101-145, 301-341, 501-521, or 601-682; and
(b) an RNA guide (or a nucleic acid encoding the RNA guide) comprising a direct repeat sequence and a spacer sequence capable of hybridizing to the target nucleic acid;
wherein the CRISPR-associated protein is capable of binding to the RNA guide; and
wherein recognition of the target nucleic acid by the CRISPR-associated protein and RNA guide results in a modification of the target nucleic acid.

135. The method of claim 134, wherein the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence set forth in any one of SEQ ID NOs: 1, 101, 301, 501, or 601.

136. The method of claim 134, wherein the CRISPR-associated protein comprises an amino acid sequence of one of any one of SEQ ID NOs: 1, 101, 301, 501, or 601.

137. The method of any of claims 134-136, wherein the transfection is a transient transfection.

138. The method of any of claims 134-137, wherein the cell is a human cell.
